SEQUENCE LISTING (3 sequences)

SEQ ID NO: 1 - protein, 6 aa, Homo sapiens
QCXXCG

SEQ ID NO: 2 - protein, 23 aa, Homo sapiens
QCXXCGXXXFXXXXXLXXHXXXH

SEQ ID NO: 3 - protein, 17 aa, Homo sapiens
QCXXCXXCGXXCXXXXX

(19) United States
(12) Patent Application Publication
(10) Pub. No.: US 2024/0018118 A1
(43) Pub. Date: Jan. 18, 2024

(54) TRICYCLIC COMPOUNDS TO DEGRADE NEOSUBSTRATES FOR MEDICAL THERAPY

(71) Applicant: C 4 Therapeutics, Inc., Watertown, MA (US)

(72) Inventors: Christopher G. Nasveschuk, Stoneham, MA (US); Corey Don Anderson, Brighton, MA (US); James A. Henderson, Weston, MA (US); Victoria Garza, San Antonio, TX (US); Moses Moustakim, Cambridge, MA (US); Morgan Welzel O'shea, Somerville, MA (US)

(73) Assignee: C 4 Therapeutics, Inc., Watertown, MA (US)

(21) Appl. No.: 18/134,990

(22) Filed: Apr. 14, 2023

Related U.S. Application Data
(63) Continuation of application No. PCT/US21/55104, filed on Oct. 14, 2021.
(60) Provisional application No. 63/091,894, filed on Oct. 14, 2020.

(51) Int. Cl. C07D 401/04; C07D 471/06; C07D 487/06; C07D 498/06; C07D 413/04; C07D 417/04; C07D 403/04; C07D 491/107; C07D 513/20; C07D 209/92; C07F 5/02; C07D 401/14; C07D 498/08; C07D 405/14; C07D 417/14

(52) U.S. Cl. CPC C07D 401/04; C07D 417/14; C07D 487/06; C07D 498/06; C07D 413/04; C07D 417/04; C07D 403/04; C07D 491/107; C07D 513/20; C07D 209/92; C07F 5/025; C07D 401/14; C07D 498/08; C07D 405/14; C07D 471/06

(57) ABSTRACT

The invention provides tricyclic compounds that degrade neosubstrates for use in the treatment of disorders described herein, including, for example, abnormal cellular proliferation, neurodegenerative diseases, and autoimmune diseases.

### CROSS-REFERENCE TO RELATED APPLICATIONS

This application is a continuation of International Patent Application No. PCT/US2021/055104, filed on Oct. 14, 2021, which claims benefit of and priority to U.S. Provisional Application No. 63/091,894, filed on Oct. 14, 2020, each of which is hereby incorporated by reference herein in its entirety.

### FIELD OF THE INVENTION

The invention provides tricyclic compounds that degrade cereblon E3 ubiquitin ligase neosubstrates for use in the treatment of disorders described herein, including, for example, abnormal cellular proliferation, inflammatory disorders, neurodegenerative diseases, and autoimmune diseases.

### INCORPORATION BY REFERENCE

The contents of the XML file named “16010-058WO1US1_ST26_2023-03-23.xml” which was created on Mar. 23, 2023, and is 3.66 KB in size, are hereby incorporated by reference in their entirety.

### BACKGROUND

Protein degradation is a highly regulated and essential process that maintains cellular homeostasis. The selective identification and removal of damaged, misfolded, or excess proteins is achieved via the ubiquitin-proteasome pathway (UPP). The UPP is central to the regulation of almost all cellular processes, including antigen processing, apoptosis, biogenesis of organelles, cell cycling, DNA transcription and repair, differentiation and development, immune response and inflammation, neural and muscular degeneration, morphogenesis of neural networks, modulation of cell surface receptors, ion channels and the secretory pathway, the response to stress and extracellular modulators, ribosome biogenesis and viral infection.

Covalent attachment of multiple ubiquitin molecules by an E3 ubiquitin ligase to a terminal lysine residue marks the protein for proteasome degradation, where the protein is digested into small peptides and eventually into its constituent amino acids that serve as building blocks for new proteins. Defective proteasomal degradation has been linked to a variety of clinical disorders including Alzheimer's disease, Parkinson's disease, Huntington's disease, muscular dystrophies, cardiovascular disease, and cancer among others.

The drug thalidomide and its analogs lenalidomide and pomalidomide have garnered interest as immunomodulators and antineoplastics, especially in multiple myeloma (see Martiniani, R. et al. “Biological activity of lenalidomide and its underlying therapeutic effects in multiple myeloma” Adv Hematol, 2012, 2012:842945; and Terpos, E. et al. “Pomalidomide: a novel drug to treat relapsed and refractory multiple myeloma” Oncotargets and Therapy, 2013, 6:531). Thalidomide, lenalidomide, pomalidomide, and analogues thereof contain an imid functionality (C(O)—NH—C(O)). Celegene has disclosed various imides and their uses, including those in U.S. Pat. Nos. 6,045,501; 6,315,720; 6,395,754; 6,561,976; 6,561,977; 6,755,784; 6,869,399; 6,908,432; 7,141,018; 7,230,012; 7,820,697; 7,874,984; 7,959,566; 8,204,763; 8,315,886; 8,589,188; 8,626,531; 8,673,939; 8,735,428; 8,741,929; 8,828,427; 9,056,120; 9,101,621; and 9,101,622.

The phthalimide portion of the imide interacts with specific amino acids in the cereblon receptor of the ligase protein complex to “create” a thermodynamically favorable site for what is referred to as a “neosubstrate”, which is a protein that would not normally bind to the ligase but for the binding of the drug to cereblon to create this new site. A significant focus of current research is the identification of neosubstrates based on various chemical structures of cereblon binding ligands. This opens a new means to intercept dysfunctional disease-causing biological pathways that rely on protein neosubstrates, and with that, new paths for medical therapies.

The rapid ubiquitination and proteasomal degradation of IKZF1 and IKZF3 by thalidomide, lenalidomide and pomalidamide has been the subject of extensive research. The drugs recruit IKZF⅓ to the CRL4^{CRBN} E3 ubiquitin ligase through the Cys2-His2 (C2H2) zinc finger (ZF) domains in both IKZF1 and IKZF3. Sievers et.al., Defining the human C2H2 zinc finger degrome targeted by thalidomide analogs through CRBN, Science 362, 558 (2018). Sievers et.al. tested a large number of proteins with C2H2 zinc finger domains and identified 15 individual ZFs and seven full-length ZF-containing proteins that are degraded by thalidomide derivatives in functional or computational screens. The work showed that 28 ZFs (including IKZF2 and IKZF4) with diverse amino acid sequences bind the same drug-CRBN interface. They observed that thalidomide analogs with chemical modifications at the drug-ZF interface are capable of converting ZFs with weak affinity for the CRBN-pomalidomide complex into degraded targets. IKZF2 and IKZF4 are not degraded by pomalidomide, lenalidomide or CC-122 but are efficiently degraded by CC-220, illustrating the currently unpredictable aspects of protein degradation, and the fact that neosubstrate binding motifs are uniquely based on the combination of cereblon with specific chemical structures of drugs, that create the thermodynamically favorable binding site for the neosubstrate.

Thalidomide analogues have been reported to degrade seemingly structurally unrelated proteins, further leading to questions about how cereblon works and how best to exploit it for therapeutic purposes. For example, in addition to IKZF⅓, it has been reported that casein kinase 1α (CK1α) and GSPT1 can be degraded using this mechanism. Kronke, et al., Lenalidomide induces ubiquitination and degradation of CK1α in de(5q) MDS; Nature, 523, 183-188 (2015); Matyskiela, et al., A novel cereblon modulator recruits GSPT1 to the CRL4(CRBN) ubiquitin ligase, Nature 535, 252-257 (2016); Petzold, et.al., Structural basis of lenalidomide-induced CK1α degradation by the CRL4(CRBN) ubiquitin ligase, Nature, 532, 127-130 (2016); Fischer, et.al., Structure of the DDB1-CRBN E3 ubiquitin ligase in complex with thalidomide, Nature, 512, 49-53 (2014).

It has further been reported that ARID2 can be degraded using the CRBN proteasomal pathway. Yamamoto, et.al., ARID2 is a pomalidomide-dependent CRL4^{CRBN} substrate in multiple myeloma cells, Nature Chemical Biology, published online Sep. 21, 2020. ARID2 is a component of the polybromo-associated BAF (PBAF) chromatin-remodeling complex. Yamamoto et.al., reported that ARID2 is a pomalidomide-induced neosubstrate of CRL4^{CRBN}. BRD7, another subunit of PBAF, is critical for pomalidomide-induced ARID2 degradation. The ARID2 degradation is an example of cofactor-influenced target protein degradation.

WO2020/006262 filed by Dana Farber Cancer Institute discloses tricyclic glutarimide containing compounds. WO2020/206424; WO2020/010177; and WO2020/010227 each of which was filed by Kymera also discloses tricyclic glutarimide containing compounds.

PCT/US2019/24094 and PCT/US2020/02678 filed by C4 Therapeutics, Inc. discloses cereblon binders for degradation of Ikaros (IKZF⅓).

WO 2021/127586 filed by Calico Life Sciences LLC and AbbVie Inc. describes PTPN1 and PTPN2 degraders covalently bound to various cereblon ligands.

Examples of patent applications in the zinc finger degradation space include WO 2020/012334; WO 2020/012337; WO 2019/038717; WO 2020/128972; WO 2020/006264; WO 2020/117759; WO 2021/087093; WO 2021/101919; and WO 2021/194914.

Despite these efforts there remains a need for new compounds, uses and processes of manufacture for medical therapy, including for the treatment of abnormal cellular proliferation, neurodegenerative diseases, and autoimmune diseases, wherein the compounds can bind to the cereblon receptor of CRL4^{CRBN} E3 ubiquitin ligase to create new binding sites for neosubstrates that are mediators of human disease, in a manner that causes the protein degradation of the neosubstrate.

### SUMMARY OF THE INVENTION

New tricyclic compounds are provided, along with their uses and manufacture, for the treatment of diseases as described herein, for example, diseases characterized by abnormal cellular proliferation, neurodegenerative diseases, inflammatory diseases and autoimmune diseases.

The tricyclic compounds provided herein can bind to the cereblon receptor of CRL4^{CRBN}E3 ubiquitin ligase to create new binding sites for protein neosubstrates that are mediators of human disease, in a manner that causes the protein degradation of the neosubstrate. The tricyclic compound described herein creates a neomorphic surface on cereblon that can interact directly with a target protein or target protein complex to directly or indirectly reduce protein levels. In varying embodiments, the tricyclic compounds described herein can generate a reduction in a neosubstrate target protein level via direct ubiquitination of the target protein; or ubiquitination of a neosubstrate target protein cofactor or target protein complex or other protein responsible for controlling target protein homeostasis. The compounds may cause the degradation of neosubstrate target proteins that directly bind ligand-bound cereblon; the degradation of a neosubstrate that is a cofactor that binds ligand-bound cereblon; degradation where a composite cofactor and target protein interface binds ligand-bound cereblon; the degradation of a neosubstrate target protein complex that binds ligand-bound CRBN; or the reduction of a target protein level by degradation of a protein that influences the homeostasis level of the target protein but is not in the complex or a cofactor of the target protein.

In certain embodiments, the degraded neosubstrate is a protein with a β-hairpin turn containing a glycine at a key position (a “g-loop protein” or “g-loop degron”) that acts as a “structural degron” for cereblon when the cereblon is also bound to the tricyclic compound of the present invention, as described further herein. Non-limiting examples of neosubstrates include Sal-like protein 4 (SALL4), GSPT1, IKFZ1, IKFZ3, CK1α, ZFP91, ZNF93, a protein kinase, C2H2 containing zinc finger protein, an RNA-recognition motif containing protein, a zinc beta ribbon containing protein, a beta-propeller containing protein, a P-loop NTPase containing protein, a really interesting new gene (RING)-finger domain containing protein, an SRC Homology 3 (SH3)-domain containing protein, an immunoglobulin E-set domain containing protein, a Tudor-domain containing protein, FAM38 or ARID. In other embodiments, another disease-mediating protein is degraded by the disclosed tricyclic cereblon-binding compound, including any of those described herein or as otherwise determined.

In another embodiment, the tricyclic compounds that bind to the cereblon receptor of CRL4^{CRBN} E3 ubiquitin ligase can create new binding sites for more than one protein neosubstrate that is a mediator of human disease, in a manner that causes the protein degradation of multiple neosubstrates. In certain aspects both IRAK4 and IKZF are degraded. In another embodiment, both SALL4 and IKZF are degraded. In other embodiments, other variations of multiple proteins that are described herein are degraded in a fashion that treats the target human disease.

In a principal embodiment, a selected tricyclic compound of Formula I, Formula II, Formula III, Formula IV, Formula V, Formula VI, Formula VII, Formula VIII, Formula IX, Formula X, Formula XI, Formula XII, Formula XIII, or Formula XIV can be provided to a host such as a human in need thereof in an effective amount to treat any of the disorders described herein:

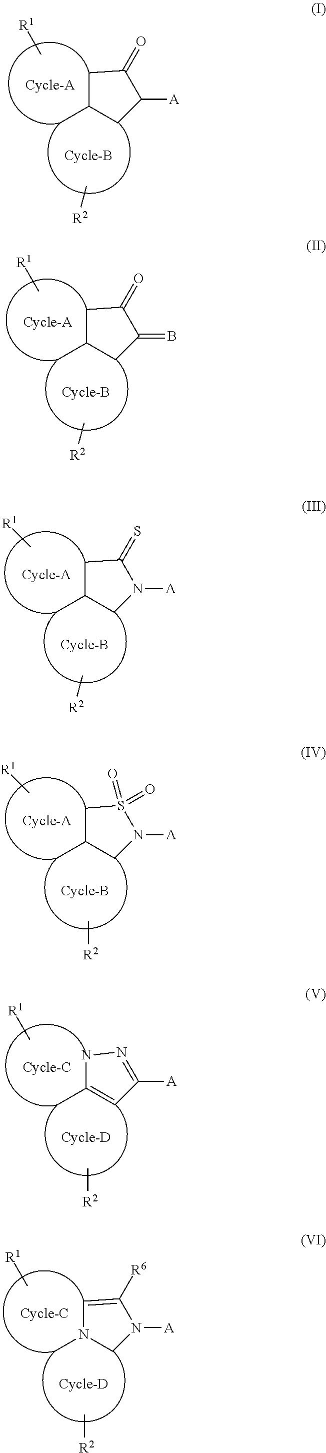

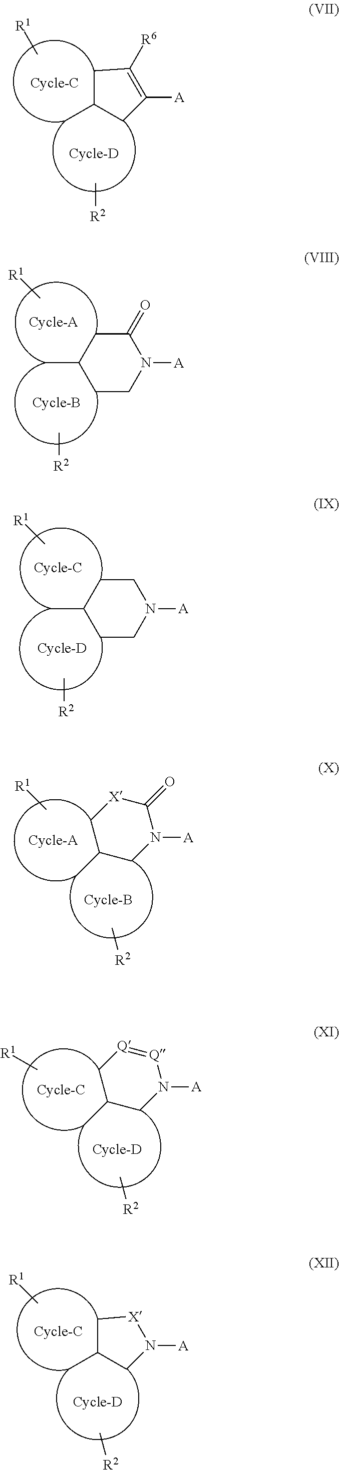

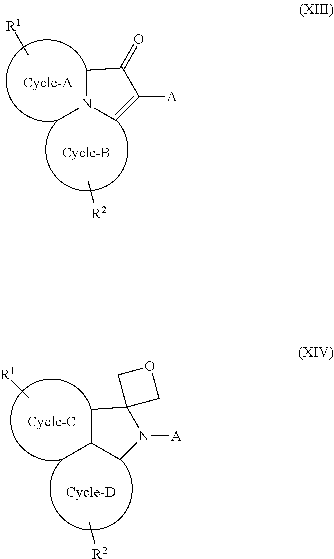

or a pharmaceutically acceptable salt, N-oxide, isotopic derivative, or prodrug thereof, optionally in a pharmaceutically acceptable carrier to form a composition;
wherein:

A is selected from

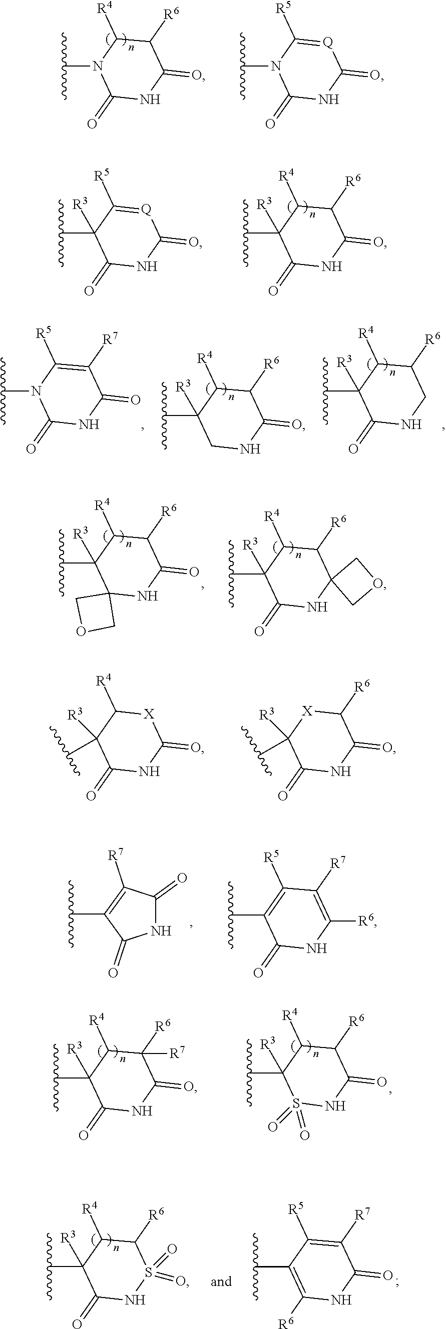

B is selected from

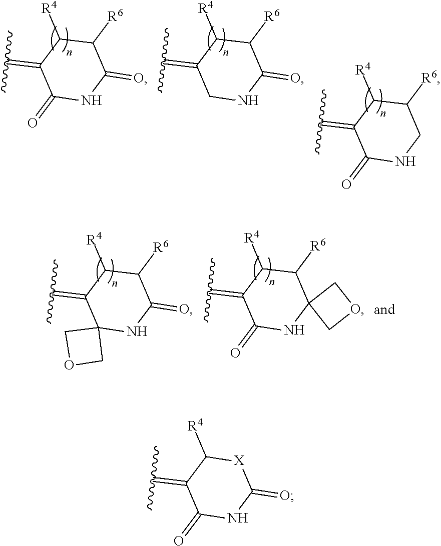

n is 0, 1, or 2;
X is NR¹⁰, NR⁶″, O, or S;
X′ is NR¹⁰, O, CH₂ or S;
Q is CR⁷ r N;
Q′ and Q″ are each independently selected from CR⁶ and N;
Cycle-A is a fused ring selected from phenyl, 5- or 6-membered heteroaryl, 5- to 8-membered heterocycle, 5- to 8-membered cycloalkyl, and 5- to 8-membered cycloalkenyl wherein Cycle-A is optionally substituted with 1, 2, or 3 substituents independently selected from R¹ as allowed by valence;
Cycle-B is a fused ring selected from phenyl, 5- or 6-membered heteroaryl, 5- to 8-membered heterocycle, 5- to 8-membered cycloalkyl, and 5- to 8-membered cycloalkenyl wherein Cycle-B is optionally substituted with 1, 2, or 3 substituents independently selected from R² as allowed by valence;
In certain embodiments Cycle-A is a fused ring selected from phenyl, 5- or 6-membered heteroaryl, 5- to 6-membered heterocycle, 5- to 6-membered cycloalkyl, or 5- to 6-membered cycloalkenyl, wherein Cycle-A is optionally substituted with 1, 2, or 3 substituents independently selected from R¹ as allowed by valence;
In certain embodiments Cycle-B is a fused ring selected from phenyl, 5- or 6-membered heteroaryl, 5- to 6-membered heterocycle, 5- to 6-membered cycloalkyl, or 5- to 6-membered cycloalkenyl, wherein Cycle-B is optionally substituted with 1, 2, or 3 substituents independently selected from R² as allowed by valence;
Cycle-C is a fused ring selected from phenyl, 5- or 6-membered heteroaryl, 5- to 6-membered heterocycle, 5- to 6-membered cycloalkyl, and 5- to 6-membered cycloalkenyl wherein Cycle-C is optionally substituted with 1, 2, or 3 substituents independently selected from R¹ as allowed by valence;
Cycle-D is a fused ring selected from phenyl, 5- or 6-membered heteroaryl, 5 to 6-membered heterocycle, 5- to 6-membered cycloalkyl, and 5- to 6-membered cycloalkenyl wherein Cycle-D is optionally substituted with 1, 2, or 3 substituents independently selected from R² as allowed by valence;
R¹ and R² are each independently at each instance selected from
(a) hydrogen, alkyl, halogen, haloalkyl, —OR¹⁰, —SR¹⁰, —S(O)R¹², —SO₂R¹², —NR¹⁰R¹¹, cyano, nitro, heteroaryl, aryl, cycloalkyl, and heterocycle wherein each heteroaryl, aryl, cycloalkyl, and heterocycle is optionally substituted with 1, 2, 3, or 4 substituents independently selected from R⁴⁰;
(b)

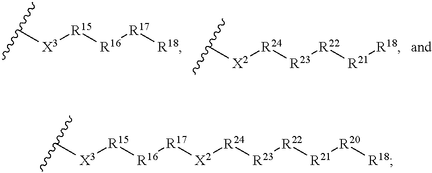

and
(c) if allowed by valence and stability, a divalent moiety such as O, S, or ═NR²⁵; and
wherein an R group may optionally be combined with another R¹ group or an R² group to form a fused cycle or bicycle which may bridge Cycle-A and Cycle-B or Cycle-C and Cycle-D, as appropriate and desired;
R³ is hydrogen, alkyl, halogen, or haloalkyl;
or R³ and R⁶ are combined to form a 1 or 2 carbon attachment, for example, when R³ and R⁶ form a 1 carbon attachment

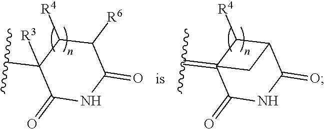

or R³ and R⁴ are combined to form a 1, 2, 3, or 4 carbon attachment, for example when R³ and R⁴ form a 1 carbon attachment

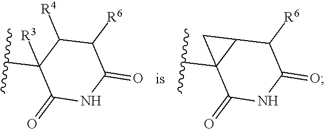

or R³ and an R⁴ group adjacent to R³ are combined to form a double bond;
each R⁴ is independently selected from hydrogen, alkyl, halogen, and haloalkyl;
R⁵ is hydrogen, alkyl, halogen, or haloalkyl;
R⁶ and R⁷ are independently selected at each instance from hydrogen, alkyl, halogen, haloalkyl, —OR¹⁰, —SR¹⁰, —S(O)R¹², —SO₂R¹², and —NR¹⁰R¹¹; wherein if R⁶ and R⁷ are on the same carbon atom they can optionally form a 3- to 4-membered spirocycle ring.
R^{6′} is hydrogen, alkyl, or haloalkyl;
or R³ and R^{6′} are combined to form a 1 or 2 carbon attachment, for example when R³ and R^{6′} form a 1 carbon attachment

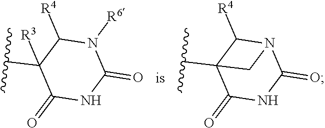

each R¹⁰ and R¹¹ are independently selected from hydrogen, alkyl, haloalkyl, heterocycle, aryl, heteroaryl, —C(O)R¹², —S(O)R¹², and —SO₂R¹²;
each R¹² is independently selected from hydrogen, alkyl, haloalkyl, heterocycle, aryl, heteroaryl, —NR¹³R¹⁴, and OR¹³;
R¹³ and R¹⁴ are each independently selected from hydrogen, alkyl, and haloalkyl;
each X² is a bivalent moiety selected from bond, heterocycle, aryl, heteroaryl, bicycle, alkyl, aliphatic, heteroaliphatic, —NR²⁷—, —CR⁴⁰R⁴¹—, —O—, —C(O)—, —C(NR²⁷)—, —C(S)—, —S(O)—, —S(O)₂—, and —S—; each of which heterocycle, aryl, heteroaryl, and bicycle is optionally substituted with 1, 2, 3, or 4 substituents independently selected from R⁴⁰;
X³ is a bivalent moiety selected from bond, heterocycle, aryl, heteroaryl, bicycle, —NR²⁷—, CR⁴⁰R⁴¹—, —O—, —C(O)—, —C(NR²⁷)—, —C(S)—, —S(O)—, —S(O)₂—, —S—, arylalkyl, heterocyclealkyl, or heteroarylalkyl (in either direction); each of which heterocycle, aryl, heteroaryl, and bicycle may be substituted with 1, 2, 3, or 4 substituents independently selected from R⁴⁰;
R¹⁵, R¹⁶, and R¹⁷ are independently at each occurrence selected from the group consisting of a bond, alkyl (which in certain embodiments is a carbocycle), —C(O)—, —C(O)O—, —OC(O)—, —SO₂—, —S(O)—, —C(S)—, —C(O)NR²⁷—, —NR²⁷C(O)—, —O—, —S—, —NR²⁷—, —C(R⁴⁰R⁴¹)—, —P(O)(OR²⁶)O—, —P(O)(OR²⁶)—, bicycle, alkene, alkyne, haloalkyl, alkoxy, aryl, heterocycle, aliphatic, heteroaliphatic, heteroaryl, lactic acid, glycolic acid arylalkyl, heterocyclealkyl and heteroarylalkyl (in either direction); each of which is optionally substituted with 1, 2, 3, or 4 substituents independently selected from R⁴⁰;
R¹⁸ is selected from hydrogen, alkyl, alkene, alkyne, hydroxy, azide, amino, halogen, haloalkyl, —OR¹⁰, —SR¹⁰, —S(O)R¹², —SO₂R¹², —NR¹⁰R¹¹, cyano, nitro, heteroaryl, aryl, arylalkyl, cycloalkyl, and heterocycle wherein each heteroaryl, aryl, arylalkyl, cycloalkyl, and heterocycle is optionally substituted with 1, 2, 3, or 4 substituents independently selected from R⁴⁰;
R²⁰, R²¹, R²², R²³, and R²⁴ are independently at each occurrence selected from the group consisting of a bond, alkyl, —C(O)—, —C(O)O—, —OC(O)—, —SO₂—, —S(O)—, —C(S)—, —C(O)NR²⁷—, —NR²⁷C(O)—, —O—, —S—, —NR²⁷—, oxyalkylene, —C(R⁴⁰R⁴⁰)—, —P(O)(OR²⁶)O—, —P(O)(OR²⁶)—, bicycle, alkene, alkyne, haloalkyl, alkoxy, aryl, heterocycle, aliphatic, heteroaliphatic, heteroaryl, lactic acid, glycolic acid, and carbocycle; each of which is optionally substituted with 1, 2, 3, or 4 substituents independently selected from R⁴⁰;
R²⁵ is aliphatic (including alkyl), aryl, heteroaryl, or hydrogen;
R²⁶ is independently at each occurrence selected from the group consisting of hydrogen, alkyl, arylalkyl, heteroarylalkyl, alkene, alkyne, aryl, heteroaryl, heterocycle, aliphatic and heteroaliphatic;
R²⁷ is independently at each occurrence selected from the group consisting of hydrogen, alkyl, aliphatic, heteroaliphatic, heterocycle, aryl, heteroaryl, —C(O)(aliphatic, aryl, heteroaliphatic or heteroaryl), —C(O)O(aliphatic, aryl, heteroaliphatic, or heteroaryl), alkene, and alkyne;
R⁴⁰ is independently at each occurrence selected from the group consisting of hydrogen, R²⁷, alkyl, alkene, alkyne, fluoro, bromo, chloro, hydroxyl, alkoxy, azide, amino, cyano, —NH(aliphatic, including alkyl), —N(aliphatic, including alkyl)₂, —NHSO₂(aliphatic, including alkyl), —N(aliphatic, including alkyl)SO₂alkyl, —NHSO₂(aryl, heteroaryl or heterocycle), —N(alkyl)SO₂(aryl, heteroaryl or heterocycle),—NHSO₂alkenyl, —N(alkyl)SO₂alkenyl, —NHSO₂alkynyl, —N(alkyl)SO₂alkynyl, haloalkyl, aliphatic, heteroaliphatic, aryl, heteroaryl, heterocycle, and cycloalkyl; and
R⁴¹ is aliphatic (including alkyl), aryl, heteroaryl, or hydrogen.
In another aspect of the invention a compound of Formula XV, Formula XVI, or Formula XVII is provided:

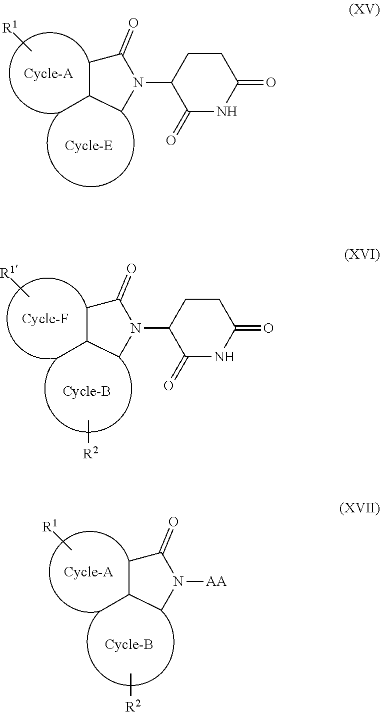

or a pharmaceutically acceptable salt, N-oxide, isotopic derivative, or prodrug thereof, optionally in a pharmaceutically acceptable carrier to form a composition;
wherein

AA is selected from

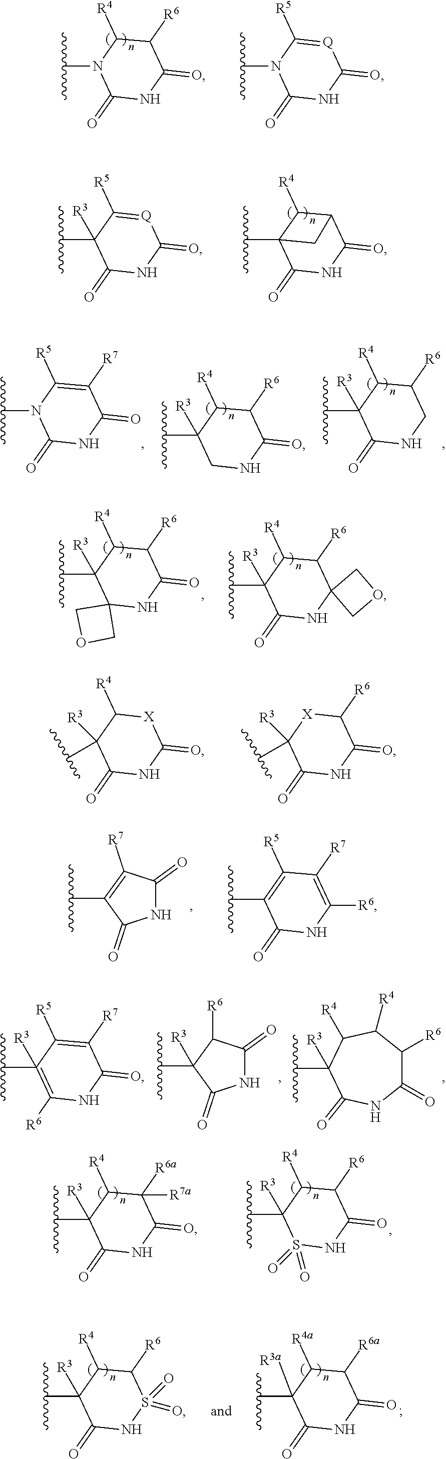

Cycle-E is selected from
(a)

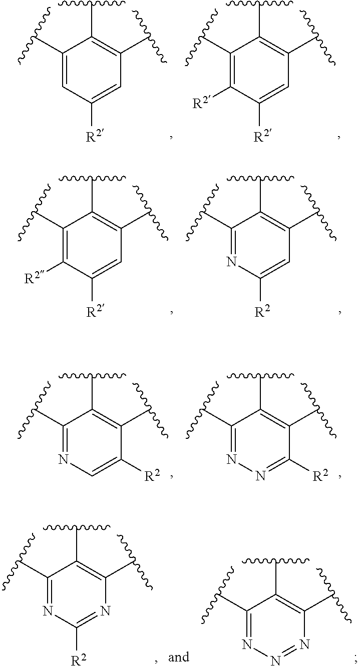

(b) a fused ring selected from 5-membered heteroaryl, 5- to 8-membered heterocycle, 5- to 8-membered cycloalkyl, or 5- to 8-membered cycloalkenyl optionally substituted with 1, 2, or 3 substituents independently selected from R² as allowed by valence;
Cycle-F is selected from
(a) phenyl substituted with 1, 2, or 3 substituents independently selected from R¹′; and
(b) a fused ring selected from 5- or 6-membered heteroaryl, 5- to 8-membered heterocycle, 5- to 8-membered cycloalkyl, or 5- to 8-membered cycloalkenyl optionally substituted with 1, 2, or 3 substituents independently selected from R¹ as allowed by valence;
R¹′ is independently at each instance selected from
(a) alkyl, halogen, haloalkyl, —OR¹⁰, —SR¹⁰, —S(O)R¹², —SO₂R¹², —NR¹⁰R¹¹, cyano, nitro, heteroaryl, aryl, cycloalkyl, and heterocycle wherein each heteroaryl, aryl and heterocycle is optionally substituted with 1, 2, 3, or 4 substituents independently selected from R⁴⁰;
(b)

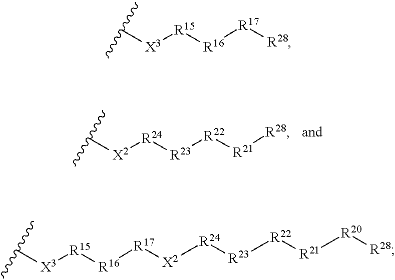

and
(c) if allowed by valence and stability, a divalent moiety such as O, S, or ═NR²⁵; and
wherein an R¹′ group may optionally be combined with another R¹′ group or an R² group to form a fused cycle or bicycle which may bridge Cycle-A and Cycle-E;
R²′ is independently at each instance selected from alkyl, halogen, haloalkyl, —OR¹⁰, —SR¹⁰, —S(O)R¹², —SO₂R¹², —NR¹⁰R¹¹, cyano, nitro, heteroaryl, aryl, and heterocycle; or alternatively, if allowed by valence and stability, R²′ may be a divalent moiety such as O, S, or ═NR²⁵; and wherein an R²′ group may optionally be combined with another R²′ group or an R¹ group to form a fused cycle or bicycle which may bridge Cycle-A and Cycle-E;
R²″ is independently at each instance selected from heteroaryl, aryl, and heterocycle, and wherein each heteroaryl, aryl and heterocycle is optionally substituted with 1, 2, 3, or 4 substituents independently selected from R⁴⁰ and wherein an R²″ group may optionally be combined with an R¹ group or an R² group to form a fused cycle or bicycle which may bridge Cycle-A and Cycle-E;
R^{3a} is hydrogen, alkyl, halogen, or haloalkyl;
or R^{3a} and R^{6a} are combined to form a 1 or 2 carbon attachment, for example, when R^{3a} and R^{6a} form a 1 carbon attachment

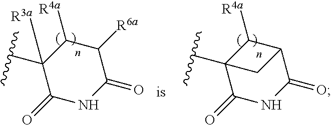

or R^{3a} and R^{4a} are combined to form a 1, 2, 3, or 4 carbon attachment, for example when
R^{3a} and R^{4a} form a 1 carbon attachment

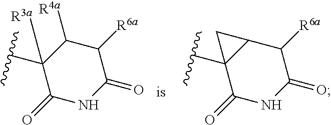

or R^{3a} and an R^{4a} group adjacent to R^{3a} are combined to form a double bond;
R^{4a} is selected from hydrogen, alkyl, halogen, and haloalkyl;
R^{6s} and R^{7a} are independently selected from hydrogen, alkyl, halogen, haloalkyl, —OR¹⁰, —SR¹⁰, —S(O)R¹², —SO₂R¹², and —NR¹⁰R¹¹;
or R^{6a} and R^{7a} are combined to form a 3-4 membered spirocyclic ring;
wherein at least one of R^{3a}, R^{4a}, R^{6a}, and R^{7a} is not hydrogen;
R²⁸ is selected from alkyl, alkene, alkyne, hydroxy, azide, amino, halogen, haloalkyl, —OR¹⁰, —SR¹⁰, —S(O)R¹², —SO₂R¹², —NR¹⁰R¹¹, cyano, nitro, heteroaryl, aryl, arylalkyl, cycloalkyl, and heterocycle wherein each heteroaryl, aryl, arylalkyl, cycloalkyl, and heterocycle is optionally substituted with 1, 2, 3, or 4 substituents independently selected from R⁴⁰;
wherein if at least one of R¹⁵, R¹⁶, R¹⁷, and R²⁰ is not bond, then R²⁸ can be hydrogen; and
wherein all other variables are as defined herein.

In some embodiments, compounds and methods are provided for the treatment of a disorder characterized by any abnormal cellular proliferation that is responsive to this therapy, including cancer, a tumor, or a non-cancerous or non-tumor condition as described more fully below. In certain embodiments, the disorder is for example hematopoietic disorder such as a lymphoid disorder, leukemia, lymphoid leukemia, lymphoblastic leukemia, acute myeloid leukemia, chronic myeloid leukemia, a hematological malignancy, multiple myeloma, a myelodysplastic syndrome such as 5q-syndrome, acute lymphoblastic leukemia, chronic lymphocytic leukemia, Hodgkin's lymphoma, non-Hodgkin's lymphoma, AML or chronic lymphocytic leukemia. In another embodiment, a selected compound of the present invention is administered to achieve immunomodulation and to reduce angiogenesis. In other embodiments, compounds and methods described herein are presented for the treatment of a disorder including, but not limited to graft-versus-host rejection, viral infection, bacterial infection, an amyloid-based proteinopathy, a proteinopathy, or a fibrotic disorder. Further, other disorders are described below which can be treated with an effective amount of a compound described herein.

In certain embodiments, any of the compounds described herein have at least one desired isotopic substitution of an atom, at an amount about the natural abundance of the isotope, i.e., enriched. In certain embodiments, the compound includes a deuterium or multiple deuterium atoms.

Other features and advantages of the present invention will be apparent from the following detailed description and claims.

Thus, the present invention includes at least the following features:
(a) a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII as described herein, or a pharmaceutically acceptable salt, isotopic derivative (including a deuterated derivative), or prodrug thereof,
(b) a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII as described herein, or a pharmaceutically acceptable salt, isotopic derivative, or prodrug thereof, for the treatment of a medical disorder responsive to the compound, as further described herein in a patient, typically a human, in need thereof;
(c) use of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII as described herein, or a pharmaceutically acceptable salt, isotopic derivative, or prodrug thereof, in an effective amount in the treatment of a patient, typically a human, with any one of the disorders described herein;
(d) use of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII as described herein, or a pharmaceutically acceptable salt, isotopic derivative, or prodrug thereof in the manufacture of a medicament for the treatment of a medical disorder responsive to the compound, as further described herein;
(e) a method of manufacturing a medicament for the treatment of a disorder described herein in a host characterized in that a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII is used in the manufacture;
(f) a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII as described herein, or a pharmaceutically acceptable salt, isotopic derivative, or prodrug thereof, for the treatment of abnormal cellular proliferation or cancer in a host, including any of the cancers described herein;
(g) use of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII as described herein, or a pharmaceutically acceptable salt, isotopic derivative, or prodrug thereof in the manufacture of a medicament for the treatment of cancer, including any of the cancers described herein;
(h) a method of manufacturing a medicament for the treatment of abnormal cellular proliferation or cancer in a host, including any of the cancers described herein, characterized in that a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII is used in the manufacture;
(i) a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII as described herein, or a pharmaceutically acceptable salt, isotopic derivative, or prodrug thereof, for the treatment of a tumor in a host, including any of the tumors described herein;
(j) use of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII as described herein, or a pharmaceutically acceptable salt, isotopic derivative, or prodrug thereof in the manufacture of a medicament for the treatment of a tumor, including any of the tumors described herein;
(k) a method of manufacturing a medicament for the treatment of a tumor in a host, including any of the tumors described herein, characterized in that a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII is used in the manufacture;
(l) a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII as described herein, or a pharmaceutically acceptable salt, isotopic derivative, or prodrug thereof, for the treatment of an immune, autoimmune, neurodegenerative, fibrotic or inflammatory disorder in a host;
(m) use of a compound of Formula I, Formula II, Formula III, Formula IV, Formula V, Formula VI, Formula VII, Formula VIII, Formula IX,
(n) Formula X, Formula XI, Formula XII, Formula XIII, Formula XIV, Formula XV, Formula XVI, or Formula XVII as described herein, or a pharmaceutically acceptable salt, isotopic derivative, or prodrug thereof in the manufacture of a medicament for the treatment of an immune, autoimmune, neurodegenerative, fibrotic or inflammatory disorder;
(o) a method of manufacturing a medicament for the treatment of an immune, autoimmune, or inflammatory disorder in a host characterized in that a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII is used in the manufacture;
(p) a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII as described herein, or a pharmaceutically acceptable salt, isotopic derivative, or prodrug thereof, for the treatment of a hematological malignancy such as multiple myeloma, leukemia, lymphoblastic leukemia, chronic lymphocytic leukemia, Hodgkin's lymphoma, or non-Hodgkin's lymphoma;
(q) use of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII as described herein, or a pharmaceutically acceptable salt, isotopic derivative, or prodrug thereof in the manufacture of a medicament for the treatment of a hematological malignancy such as multiple myeloma, leukemia, lymphoblastic leukemia, chronic lymphocytic leukemia, Hodgkin's lymphoma, or non-Hodgkin's lymphoma;
(r) a method of manufacturing a medicament for the treatment of a hematological malignancy in a host such as multiple myeloma, leukemia, lymphoblastic leukemia, chronic lymphocytic leukemia, Hodgkin's lymphoma, or non-Hodgkin's lymphoma, characterized in that a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII is used in the manufacture;
(s) a pharmaceutical composition comprising an effective host-treating amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII as described herein or a pharmaceutically acceptable salt, isotopic derivative, or prodrug thereof with a pharmaceutically acceptable carrier or diluent;
(t) a compound a described herein as a mixture of enantiomers or diastereomers (as relevant), including the racemate;
(u) a compound as described herein in enantiomerically or diastereomerically (as relevant) enriched form, including an isolated enantiomer or diastereomer (i.e. greater than about 80, 85, 90, 95, 97, or 99% pure); and
(v) a process for the manufacture of therapeutic products that contain an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII as described herein.

### BRIEF DESCRIPTION OF THE FIGURES

FIG. 1 is a synthetic scheme showing non-limiting examples of syntheses that can be used with intermediate 3-(5-bromo-2-oxobenzo[cd]indol-1(2H)-yl)piperidine-2,6-dione to add a range of R¹ and/or R² groups.

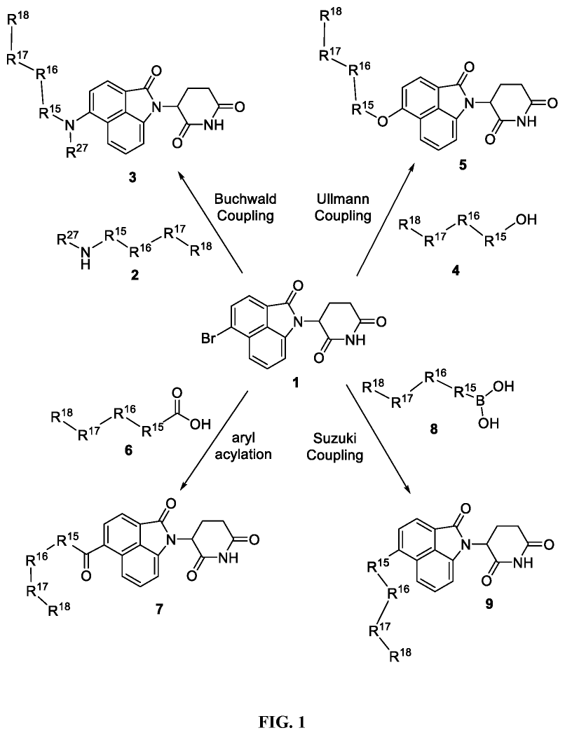

FIG. 2 is a synthetic scheme showing non-limiting examples of syntheses that can be used with intermediate 1-(2,6-dioxopiperidin-3-yl)-2-oxo-1,2-dihydrobenzo[cd]indole-5-carbaldehyde to add a range of R¹ and/or R² groups.

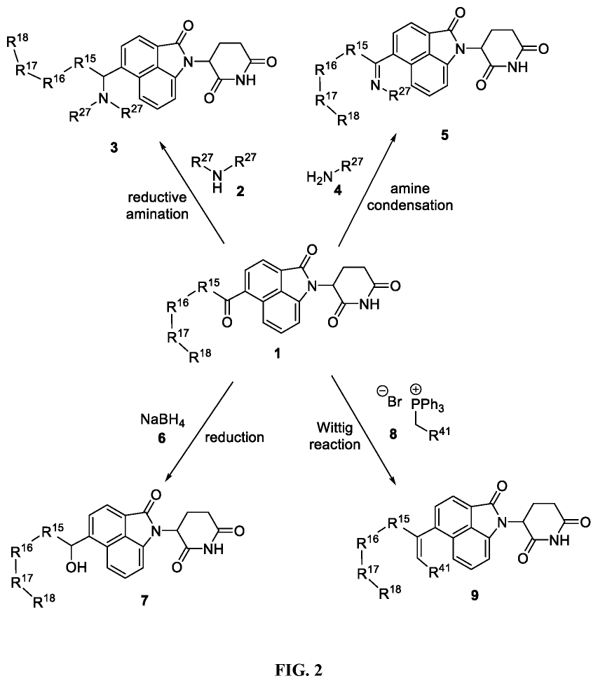

FIG. 3 is non-limiting representative formulas of compounds of the present invention.

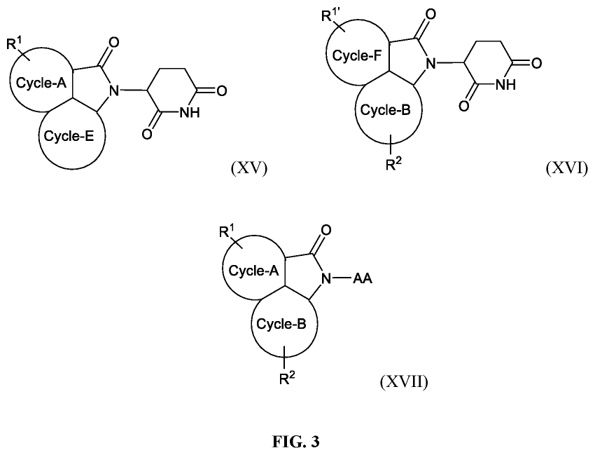

### DETAILED DESCRIPTION OF THE INVENTION

### I. Definitions

Unless otherwise defined, all technical and scientific terms used herein have the same meaning as commonly understood by one of ordinary skill in the art to which this application belongs. In the specification, singular forms also include the plural unless the context clearly dictates otherwise. Although methods and materials similar or equivalent to those described herein can be used in the practice and testing of the present application, suitable methods and materials are described below. All publications, patent applications, patents, and other references mentioned herein are incorporated by reference. The references cited herein are not admitted to be prior art to the claimed application. In the case of conflict, the present specification, including definitions, will control. In addition, the materials, methods, and examples are illustrative only and are not intended to be limiting.

Compounds are described using standard nomenclature. Unless defined otherwise, all technical and scientific terms used herein have the same meaning as is commonly understood by one of skill in the art to which this invention belongs.

In certain embodiments of each compound described herein, the compound may be in the form of a racemate, enantiomer, mixture of enantiomers, diastereomer, mixture of diastereomers, tautomer, N-oxide, or isomer, such as a rotamer, as if each is specifically described unless specifically excluded by context.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed.

The present invention includes compounds described herein with at least one desired isotopic substitution of an atom, at an amount above the natural abundance of the isotope, i.e., enriched. Isotopes are atoms having the same atomic number but different mass numbers, i.e., the same number of protons but a different number of neutrons. If isotopic substitutions are used, the common replacement is at least one deuterium for hydrogen.

More generally, examples of isotopes that can be incorporated into compounds of the invention include isotopes of hydrogen, carbon, nitrogen, oxygen, fluorine, and chlorine such as ²H, ³H, ¹¹C, ¹³C, ¹⁴C, ¹⁵N, ¹⁷O, ¹⁸O, ¹⁸F, ³⁵S, and ³⁶Cl respectively. In one non-limiting embodiment, isotopically labelled compounds can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. Additionally, any hydrogen atom present in the compound of the invention may be substituted with an ¹⁸F atom, a substitution that may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (H) may be used anywhere in described structures that achieves the desired result. Alternatively, or in addition, isotopes of carbon, e.g., ¹³C and ¹⁴C, may be used.

Isotopic substitutions, for example deuterium substitutions, can be partial or complete. Partial deuterium substitution means that at least one hydrogen is substituted with deuterium. In certain embodiments, the isotope is 90, 95 or 99% or more enriched in an isotope at any location of interest. In one non-limiting embodiment, deuterium is 90, 95 or 99% enriched at a desired location.

In one non-limiting embodiment, the substitution of a hydrogen atom for a deuterium atom can be provided in any compound described herein. For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃ etc.). In certain other embodiments, when two substituents are combined to form a cycle the unsubstituted carbons may be deuterated. In certain embodiments, at least one deuterium is placed on an atom that has a bond which is broken during metabolism of the compound in vivo, or is one, two or three atoms remote form the metabolized bond (e.g., which may be referred to as an α, β or γ, or primary, secondary or tertiary isotope effect).

The compounds of the present invention may form a solvate with a solvent (including water). Therefore, in one non-limiting embodiment, the invention includes a solvated form of the compounds described herein. The term “solvate” refers to a molecular complex of a compound of the present invention (including a salt thereof) with one or more solvent molecules. Non-limiting examples of solvents are water, ethanol, isopropanol, dimethyl sulfoxide, acetone and other common organic solvents. The term “hydrate” refers to a molecular complex comprising a compound of the invention and water. Pharmaceutically acceptable solvates in accordance with the invention include those wherein the solvent may be isotopically substituted, e.g. D₂O, d₆-acetone, d₆-DMSO. A solvate can be in a liquid or solid form.

A dash (“-”) that is not between two letters or symbols is used to indicate a point of attachment for a substituent. For example, —(C═O)NH₂ is attached through carbon of the keto (C═O) group.

“Alkyl” is a branched or straight chain saturated aliphatic hydrocarbon group. In one non-limiting embodiment, the alkyl group contains from 1 to about 12 carbon atoms, more generally from 1 to about 6 carbon atoms or from 1 to about 4 carbon atoms. In one non-limiting embodiment, the alkyl contains from 1 to about 8 carbon atoms. In certain embodiments, the alkyl is C1-C2, C1-C3, C₁-C₄, C₁-C₅, or C₁-C₆. The specified ranges as used herein indicate an alkyl group having each member of the range described as an independent species. For example, the term C₁-C₆ alkyl as used herein indicates a straight or branched alkyl group having from 1, 2, 3, 4, 5, or 6 carbon atoms and is intended to mean that each of these is described as an independent species. For example, the term C1-C4 alkyl as used herein indicates a straight or branched alkyl group having from 1, 2, 3, or 4 carbon atoms and is intended to mean that each of these is described as an independent species. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane.

“Alkenyl” is a linear or branched aliphatic hydrocarbon groups having one or more carbon-carbon double bonds that may occur at a stable point along the chain. The specified ranges as used herein indicate an alkenyl group having each member of the range described as an independent species, as described above for the alkyl moiety. In one non-limiting embodiment, the alkenyl contains from 2 to about 12 carbon atoms, more generally from 2 to about 6 carbon atoms or from 2 to about 4 carbon atoms. In certain embodiments the alkenyl is C2, C2-C3, C2-C4, C₂-C₅, or C₂-C₆. Examples of alkenyl radicals include, but are not limited to ethenyl, propenyl, allyl, propenyl, butenyl and 4-methylbutenyl. The term “alkenyl” also embodies “cis” and “trans” alkenyl geometry, or alternatively, “E” and “Z” alkenyl geometry. The term “Alkenyl” also encompasses cycloalkyl or carbocyclic groups possessing at least one point of unsaturation.

“Alkynyl” is a branched or straight chain aliphatic hydrocarbon group having one or more carbon-carbon triple bonds that may occur at any stable point along the chain. The specified ranges as used herein indicate an alkynyl group having each member of the range described as an independent species, as described above for the alkyl moiety. In one non-limiting embodiment, the alkynyl contains from 2 to about 12 carbon atoms, more generally from 2 to about 6 carbon atoms or from 2 to about 4 carbon atoms. In certain embodiments the alkynyl is C2, C2-C3, C2-C4, C2-C5, or C2-C6. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

“Halo” and “Halogen” is independently fluorine, chlorine, bromine or iodine.

“Haloalkyl” is a branched or straight-chain alkyl groups substituted with 1 or more halo atoms described above, up to the maximum allowable number of halogen atoms. Examples of haloalkyl groups include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Perhaloalkyl” means an alkyl group having all hydrogen atoms replaced with halogen atoms. Examples include but are not limited to, trifluoromethyl and pentafluoroethyl.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C14 aryl”; e.g., anthracyl). “Aryl” also includes ring systems wherein the aryl ring, as defined above, is fused with one or more cycloalkyl or heterocycle groups wherein the radical or point of attachment is on the aryl ring, and in such instances, the number of carbon atoms continue to designate the number of carbon atoms in the aryl ring system. The one or more fused cycloalkyl or heterocycle groups can be a 4 to 7-membered saturated or partially unsaturated cycloalkyl or heterocycle groups.

“Arylalkyl” refers to either an alkyl group as defined herein substituted with an aryl group as defined herein or to an aryl group as defined herein substituted with an alkyl group as defined herein.

The term “heterocycle” denotes saturated and partially saturated heteroatom-containing ring radicals, wherein there are 1, 2, 3, or 4 heteroatoms independently selected from nitrogen, sulfur, boron, silicone, and oxygen. Heterocyclic rings may comprise monocyclic 3-10 membered rings, as well as 5-16 membered bicyclic ring systems (which can include bridged, fused, and spiro-fused bicyclic ring systems). It does not include rings containing —O—O—, —O—S— or —S—S-portions. Examples of saturated heterocycle groups include saturated 3- to 6-membered heteromonocyclic groups containing 1 to 4 nitrogen atoms [e.g. pyrrolidinyl, imidazolidinyl, piperidinyl, pyrrolinyl, piperazinyl]; saturated 3 to 6-membered heteromonocyclic group containing 1 to 2 oxygen atoms and 1 to 3 nitrogen atoms [e.g. morpholinyl]; saturated 3 to 6-membered heteromonocyclic group containing 1 to 2 sulfur atoms and 1 to 3 nitrogen atoms [e.g., thiazolidinyl]. Examples of partially saturated heterocycle radicals include but are not limited to, dihydrothienyl, dihydropyranyl, dihydrofuryl, and dihydrothiazolyl. Examples of partially saturated and saturated heterocycle groups include but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl.

“Heterocycle” also includes groups wherein the heterocyclic radical is fused/condensed with an aryl or carbocycle radical, wherein the point of attachment is the heterocycle ring. “Heterocycle” also includes groups wherein the heterocyclic radical is substituted with an oxo group

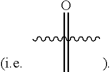 [SMILES: *=O]

For example a partially unsaturated condensed heterocyclic group containing 1 to 5 nitrogen atoms, for example, indoline or isoindoline; a partially unsaturated condensed heterocyclic group containing 1 to 2 oxygen atoms and 1 to 3 nitrogen atoms; a partially unsaturated condensed heterocyclic group containing 1 to 2 sulfur atoms and 1 to 3 nitrogen atoms; and a saturated condensed heterocyclic group containing 1 to 2 oxygen or sulfur atoms.

The term “heterocycle” also includes “bicyclic heterocycle”. The term “bicyclic heterocycle” denotes a heterocycle as defined herein wherein there is one bridged, fused, or spirocyclic portion of the heterocycle. The bridged, fused, or spirocyclic portion of the heterocycle can be a carbocycle, heterocycle, or aryl group as long as a stable molecule result. Unless excluded by context the term “heterocycle” includes bicyclic heterocycles. Bicyclic heterocycle includes groups wherein the fused heterocycle is substituted with an oxo group. Non-limiting examples of bicyclic heterocycles include:

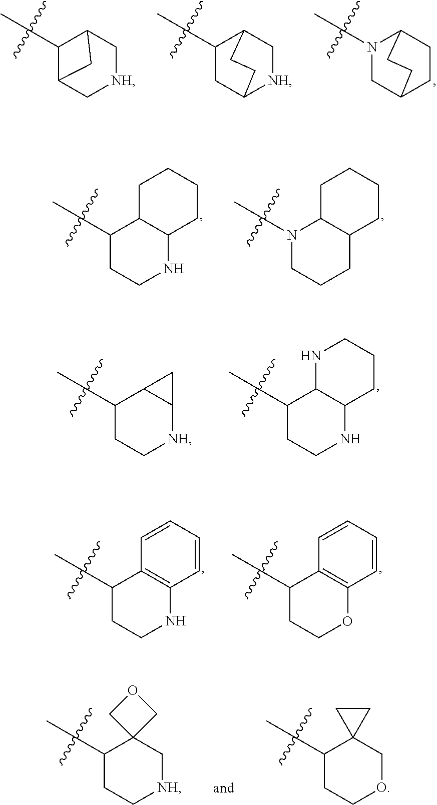

“Heterocyclealkyl” refers to either an alkyl group as defined herein substituted with a heterocycle group as defined herein or to a heterocycle group as defined herein substituted with an alkyl group as defined herein.

The term “heteroaryl” denotes stable aromatic ring systems that contain 1, 2, 3, or 4 heteroatoms independently selected from O, N, and S, wherein the ring nitrogen and sulfur atom(s) are optionally oxidized, and nitrogen atom(s) are optionally quarternized. Examples include but are not limited to, unsaturated 5 to 6 membered heteromonocyclyl groups containing 1 to 4 nitrogen atoms, such as pyrrolyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, IH-1,2,3-triazolyl, 2H-1,2,3-triazolyl]; unsaturated 5- to 6-membered heteromonocyclic groups containing an oxygen atom, for example, pyranyl, 2-furyl, 3-furyl, etc.; unsaturated 5 to 6-membered heteromonocyclic groups containing a sulfur atom, for example, 2-thienyl, 3-thienyl, etc.; unsaturated 5- to 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 to 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl]. In certain embodiments the “heteroaryl” group is a 8, 9, or 10 membered bicyclic ring system. Examples of 8, 9, or 10 membered bicyclic heteroaryl groups include benzofurazanyl, benzothiophenyl, benzothiazolyl, benzoxazolyl, quinazolinyl, quinoxalinyl, naphthyridinyl, quinolinyl, isoquinolinyl, benzofuranyl, indolyl, indazolyl, and benzotriazolyl.

“Heteroarylalkyl” refers to either an alkyl group as defined herein substituted with a heteroaryl group as defined herein or to a heteroaryl group as defined herein substituted with an alkyl group as defined herein.

As used herein, “carbocyclic”, “carbocycle” or “cycloalkyl” includes a saturated or partially unsaturated (i.e., not aromatic) group containing all carbon ring atoms and from 3 to 14 ring carbon atoms (“C₃₋₁₄ cycloalkyl”) and zero heteroatoms in the non-aromatic ring system. In some embodiments, a cycloalkyl group has 3 to 10 ring carbon atoms (“C₃₋₁₀ cycloalkyl”). In some embodiments, a cycloalkyl group has 3 to 9 ring carbon atoms (“C₃₋₉ cycloalkyl”). In some embodiments, a cycloalkyl group has 3 to 8 ring carbon atoms (“C₃₋₈ cycloalkyl”). In some embodiments, a cycloalkyl group has 3 to 7 ring carbon atoms (“C₃₋₇ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Exemplary C₃₋₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ cycloalkyl groups include, without limitation, the aforementioned C₃₋₆ cycloalkyl groups as well as cycloheptyl (C₇), cycloheptenyl (C₇), cycloheptadienyl (C₇), cycloheptatrienyl (C₇), cyclooctyl (C₅), cyclooctenyl (C₅), and the like. Exemplary C₃₋₁₀ cycloalkyl groups include, without limitation, the aforementioned C₃₋₈ cycloalkyl groups as well as cyclononyl (C₉), cyclononenyl (C₉), cyclodecyl (C₁₀), cyclodecenyl (C₁₀), and the like. As the foregoing examples illustrate, in certain embodiments, the cycloalkyl group can be saturated or can contain one or more carbon-carbon double bonds. The term “cycloalkyl” also includes ring systems wherein the cycloalkyl ring, as defined above, is fused with one heterocycle, aryl or heteroaryl ring wherein the point of attachment is on the cycloalkyl ring, and in such instances, the number of carbons continue to designate the number of carbons in the carbocyclic ring system. The term “cycloalkyl” also includes ring systems wherein the cycloalkyl ring, as defined above, has a spirocyclic heterocycle, aryl or heteroaryl ring wherein the point of attachment is on the cycloalkyl ring, and in such instances, the number of carbons continue to designate the number of carbons in the carbocyclic ring system. The term “cycloalkyl” also includes bicyclic or polycyclic fused, bridged, or spiro ring systems that contain from 5 to 14 carbon atoms and zero heteroatoms in the non-aromatic ring system. Representative examples of “cycloalkyl” include, but are not limited to,

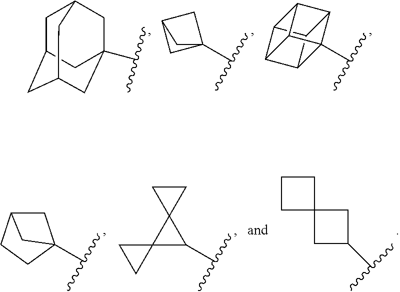

The term “bicycle” refers to a ring system wherein two rings are fused together and each ring is independently selected from carbocycle, heterocycle, aryl, and heteroaryl. Non-limiting examples of bicycle groups include:

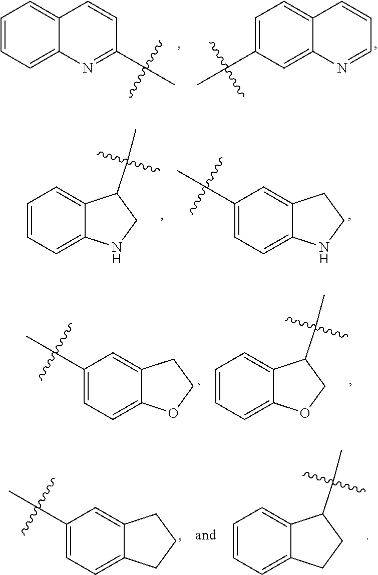

When the term “bicycle” is used in the context of a bivalent residue, the attachment points can be on separate rings or on the same ring. In certain embodiments both attachment points are on the same ring. In certain embodiments both attachment points are on different rings. Non-limiting examples of bivalent bicycle groups include:

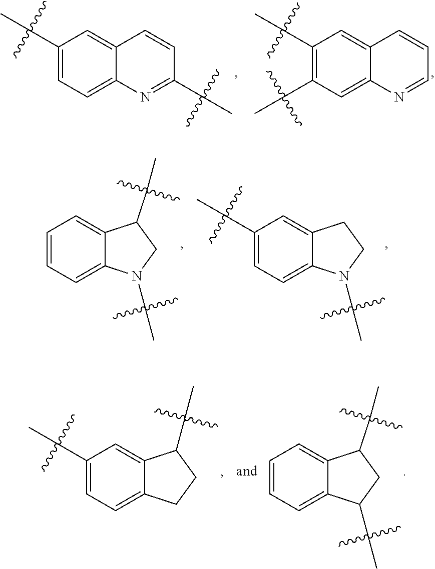 [SMILES: *c1ccc2nc(*)ccc2c1 ; *c1cc2cccnc2cc1* ; *C1CN(*)c2ccccc21 ; *c1ccc2c(c1)CCN2* ; *c1ccc2c(c1)C(*)CC2 ; *C1CC(*)c2ccccc21]

“Aliphatic” refers to a saturated or unsaturated, straight, branched, or cyclic hydrocarbon. “Aliphatic” is intended herein to include, but is not limited to, alkyl, alkenyl, alkynyl, cycloalkyl, cycloalkenyl, and cycloalkynyl moieties, and thus incorporates each of these definitions. In certain embodiments, “aliphatic” is used to indicate those aliphatic groups having 1-20 carbon atoms. The aliphatic chain can be, for example, mono-unsaturated, di-unsaturated, tri-unsaturated, or polyunsaturated, or alkynyl Unsaturated aliphatic groups can be in a cis or trans configuration. In certain embodiments, the aliphatic group contains from 1 to about 12 carbon atoms, more generally from 1 to about 6 carbon atoms or from 1 to about 4 carbon atoms. In certain embodiments, the aliphatic group contains from 1 to about 8 carbon atoms. In certain embodiments, the aliphatic group is C₁-C₂, C₁-C₃, C₁-C₄, C₁-C₅ or C₁-C₆. The specified ranges as used herein indicate an aliphatic group having each member of the range described as an independent species. For example, the term C₁-C₆ aliphatic as used herein indicates a straight or branched alkyl, alkenyl, or alkynyl group having from 1, 2, 3, 4, 5, or 6 carbon atoms and is intended to mean that each of these is described as an independent species. For example, the term C₁-C₄ aliphatic as used herein indicates a straight or branched alkyl, alkenyl, or alkynyl group having from 1, 2, 3, or 4 carbon atoms and is intended to mean that each of these is described as an independent species. In certain embodiments, the aliphatic group is substituted with one or more functional groups that results in the formation of a stable moiety.

The term “heteroaliphatic” refers to an aliphatic moiety that contains at least one heteroatom in the chain, for example, an amine, carbonyl, carboxy, oxo, thio, phosphate, phosphonate, nitrogen, phosphorus, silicon, or boron atoms in place of a carbon atom. In certain embodiments, the only heteroatom is nitrogen. In certain embodiments, the only heteroatom is oxygen. In certain embodiments, the only heteroatom is sulfur. “Heteroaliphatic” is intended herein to include, but is not limited to, heteroalkyl, heteroalkenyl, heteroalkynyl, heterocycloalkyl, heterocycloalkenyl, and heterocycloalkynyl moieties. In certain embodiments, “heteroaliphatic” is used to indicate a heteroaliphatic group (cyclic, acyclic, substituted, unsubstituted, branched or unbranched) having 1-20 carbon atoms. In certain embodiments, the heteroaliphatic group is optionally substituted in a manner that results in the formation of a stable moiety. Nonlimiting examples of heteroaliphatic moieties are polyethylene glycol, polyalkylene glycol, amide, polyamide, polylactide, polyglycolide, thioether, ether, alkyl-heterocycle-alkyl, —O-alkyl-O-alkyl, alkyl-O-haloalkyl, etc.

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, and the like. A “dosage form” can also include an implant, for example an optical implant.

As used herein “endogenous” refers to any material from or produced inside an organism, cell, tissue or system.

As used herein, the term “exogenous” refers to any material introduced from or produced outside an organism, cell, tissue or system.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

“Parenteral” administration of a compound includes, e.g., subcutaneous (s.c.), intravenous (i.v.), intramuscular (i.m.), or intrasternal injection, or infusion techniques.

As used herein, “pharmaceutical compositions” is a composition comprising at least one active agent such as a selected active compound as described herein, and at least one other substance, such as a carrier. “Pharmaceutical combinations” are combinations of at least two active agents which may be combined in a single dosage form or provided together in separate dosage forms with instructions that the active agents are to be used together to treat any disorder described herein.

As used herein, a “pharmaceutically acceptable salt” is a derivative of the disclosed compound in which the parent compound is modified by making inorganic and organic, acid or base addition salts thereof with a biologically acceptable lack of toxicity. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

The term “carrier” means a diluent, excipient, or vehicle that an active agent is used or delivered in.

A “pharmaceutically acceptable excipient” means an excipient that is useful in preparing a pharmaceutical composition/combination that is generally safe, and neither biologically nor otherwise inappropriate for administration to a host, typically a human. In certain embodiments, an excipient is used that is acceptable for veterinary use.

A “patient” or “host” or “subject” is a human or non-human animal in need of treatment, of any of the disorders as specifically described herein. Typically, the host is a human. A “host” may alternatively refer to for example, a mammal, primate (e.g., human), cow, sheep, goat, horse, dog, cat, rabbit, rat, mice, fish, bird and the like.

A “therapeutically effective amount” of a pharmaceutical composition/combination of this invention means an amount effective, when administered to a host, to provide a therapeutic benefit such as an amelioration of symptoms or reduction or diminution of the disease itself.

In certain embodiments a “prodrug” is a version of the parent molecule that is metabolized or chemically converted to the parent molecule in vivo, for example in a mammal or a human. Non-limiting examples of prodrugs include esters, amides, for example off a primary or secondary amine, carbonates, carbamates, phosphates, ketals, imines, oxazolidines, and thiazolidines. A prodrug can be designed to release the parent molecule upon a change in pH (for example in the stomach or the intestine) or upon action of an enzyme (for example an esterase or amidase).

In certain embodiments “stable” means the less than 10%, 5%, 3%, or 1% of the compound degrades under ambient conditions with a shelf life of at least 3, 4, 5, or 6-months. In certain embodiments a compound stored at ambient conditions is stored at about room temperature and exposed to air and a relative humidity of less than about 40%, 50%, 60%, or 70%. In certain embodiments a compound stored at ambient conditions is stored at about room temperature under inert gas (such as argon or nitrogen). Typically, moieties described herein do not have more than one or two heteroatoms bound to each other directly unless the moiety is heteroaromatic.

Throughout this disclosure, various aspects of the invention can be presented in a range format. It should be understood that the description in range format is merely for convenience and should not be construed as a limitation on the scope of the invention. The description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### II. Compounds of the Present Invention

In certain embodiments, the compound of Formula (I) is selected from Formula (Ia), Formula (Ib), Formula (Ic), Formula (Id), Formula (Ie), Formula (If), Formula (Ig), Formula (Ih), Formula (Ii), Formula (Ij), Formula (Ik), Formula (Il), Formula (Im), and Formula (In):

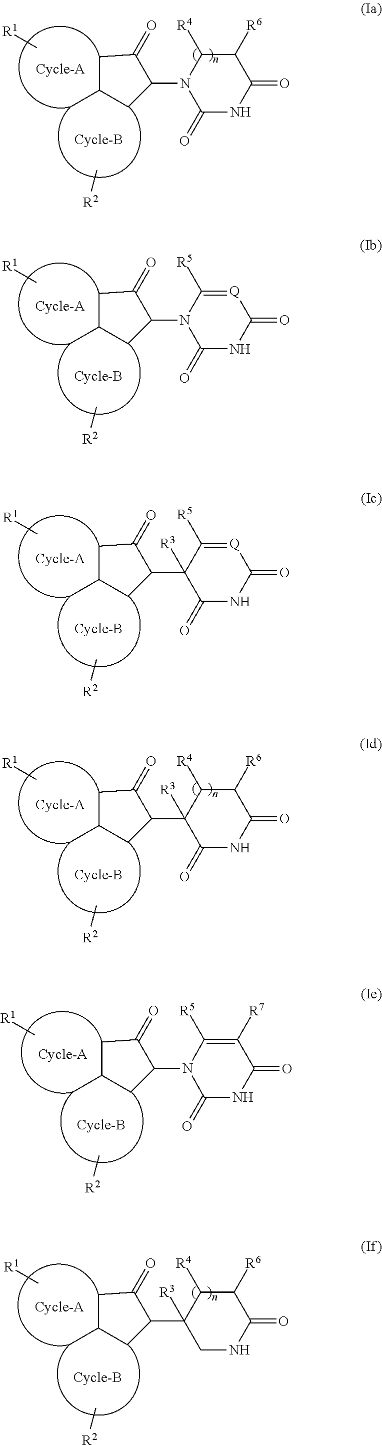

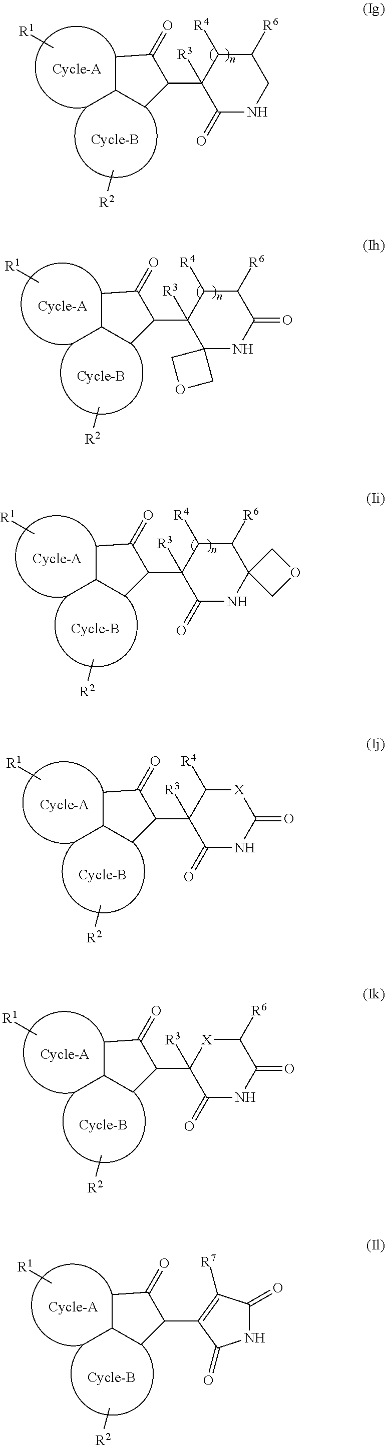

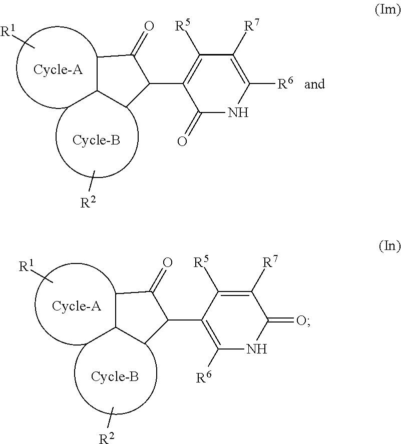

or a pharmaceutically acceptable salt thereof.

In certain embodiments, the compound of Formula (II) is selected from Formula (IIa), Formula (IIb), Formula (IIc), Formula (IId), Formula (IIe), and Formula (IIf):

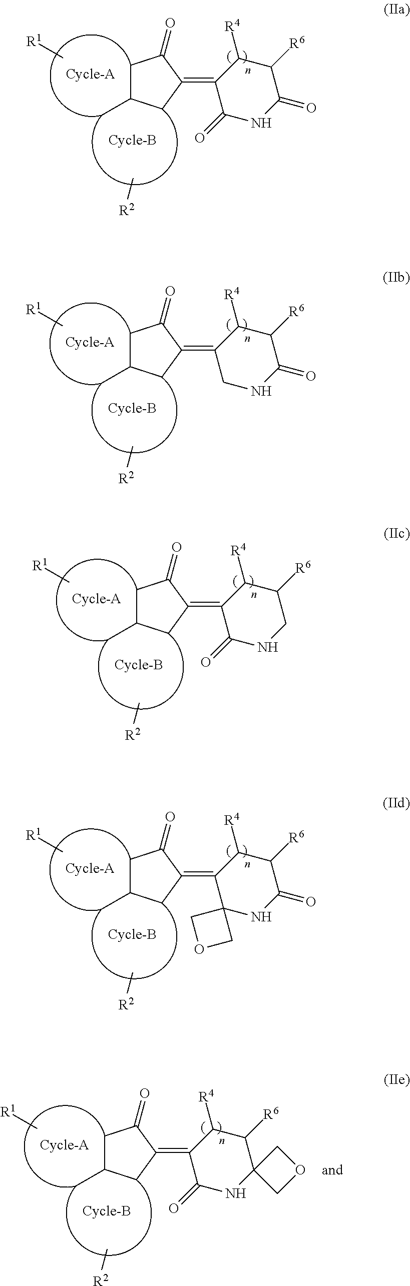

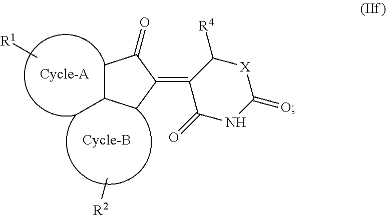

or a pharmaceutically acceptable salt thereof.

In certain embodiments, the compound of Formula (III) is selected from Formula (IIIa), Formula (IIIb), Formula (IIIc), Formula (IIId), Formula (IIIe), Formula (IIIf), Formula (IIIg), Formula (IIIh), Formula (IIIi), Formula (IIIj), Formula (IIIk), Formula (IIIl), Formula (IIIm), and Formula (TTTn)

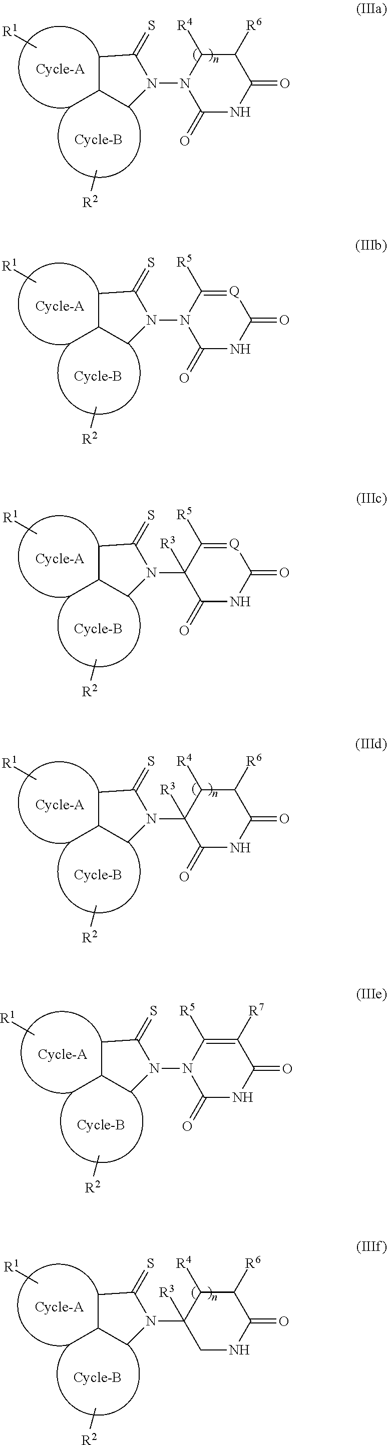

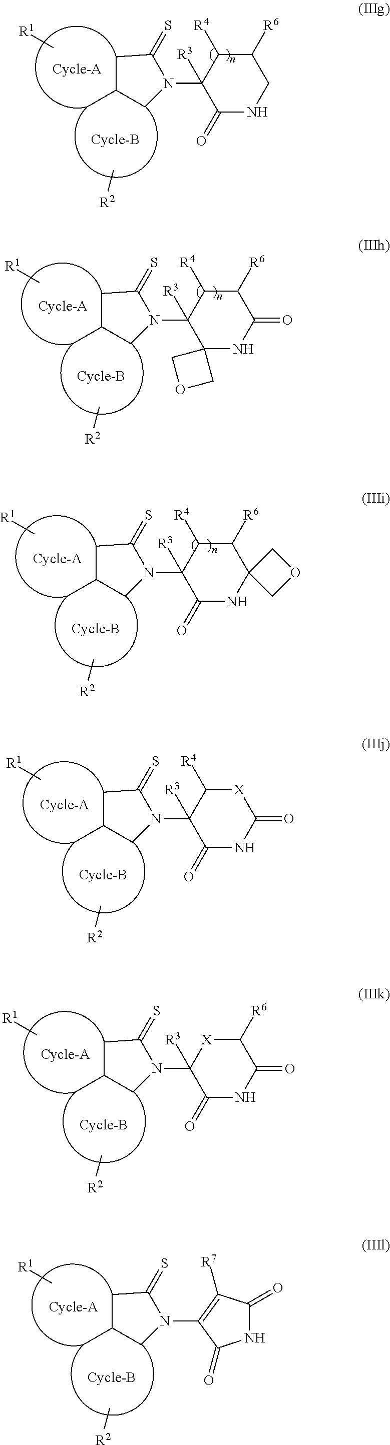

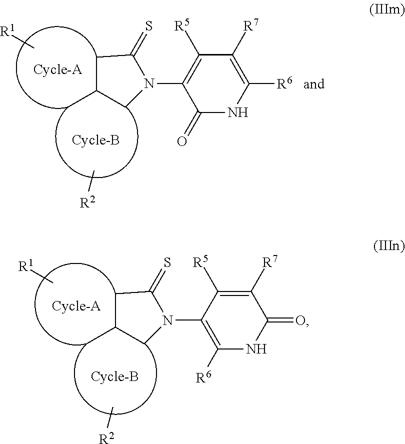

or a pharmaceutically acceptable salt thereof.

In certain embodiments, the compound of Formula (IV) is selected from Formula (IVa), Formula (IVb), Formula (IVc), Formula (IVd), Formula (IVe), Formula (IVf), Formula (IVg), Formula (IVh), Formula (IVi), Formula (IVj), Formula (IVk), Formula (IVl), Formula (IVm), and Formula (IVn):

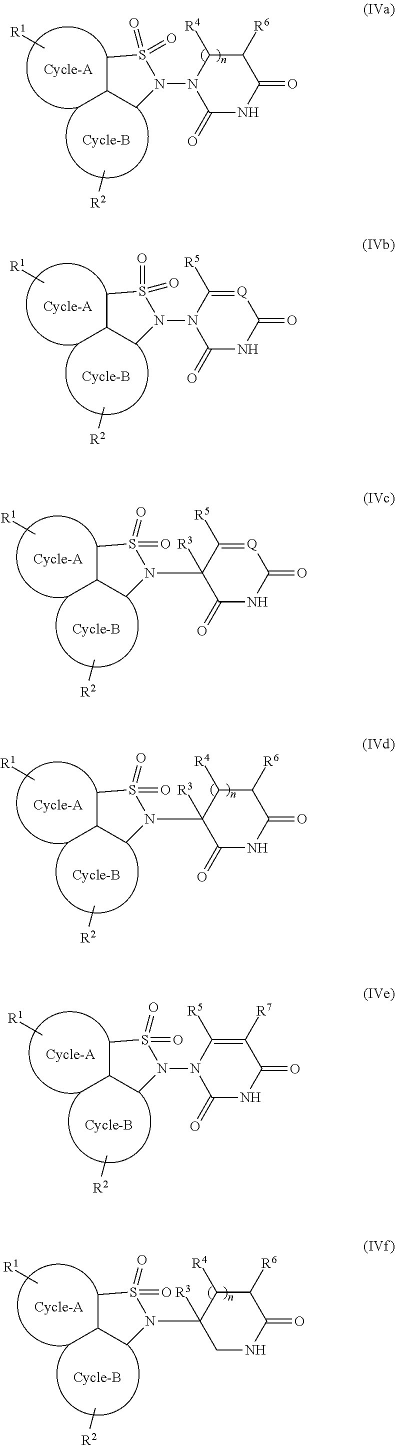

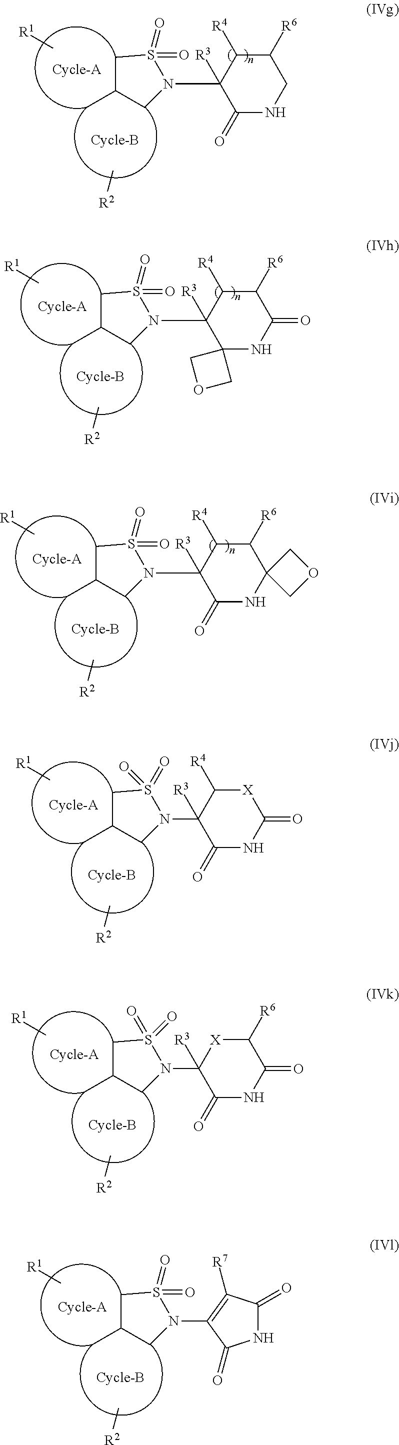

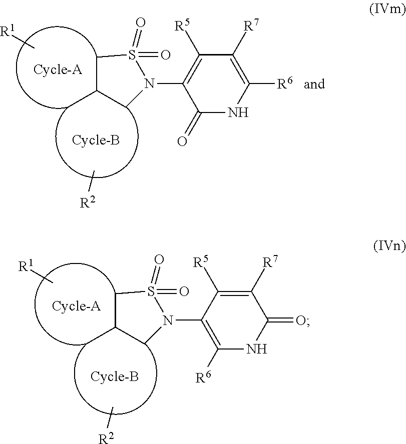

or a pharmaceutically acceptable salt thereof.

In certain embodiments, the compound of Formula (V) is selected from Formula (Va), Formula (Vb), Formula (Vc), Formula (Vd), Formula (Ve), Formula (Vf), Formula (Vg), Formula (Vh), Formula (Vi), Formula (Vj), Formula (Vk), Formula (Vl), Formula (Vm), and Formula (Vn):

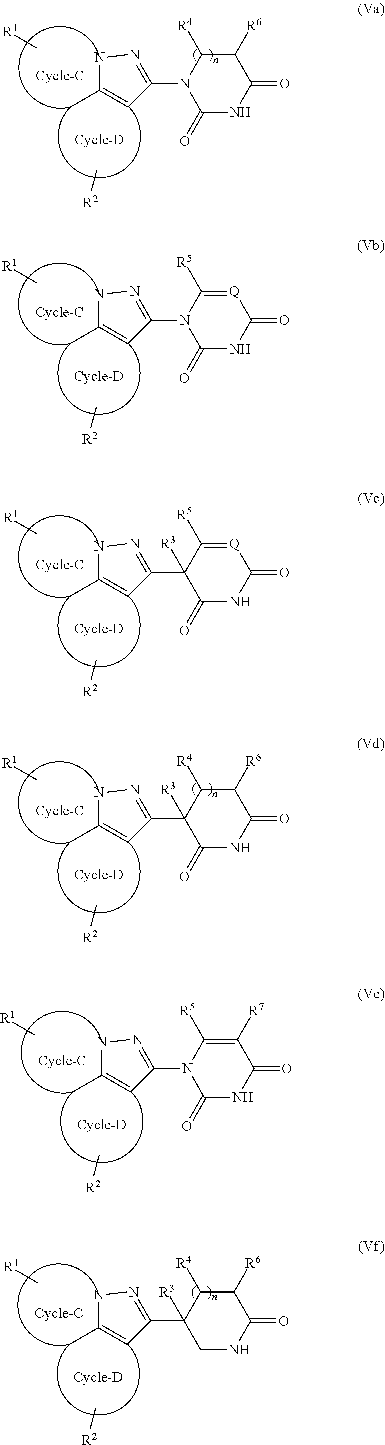

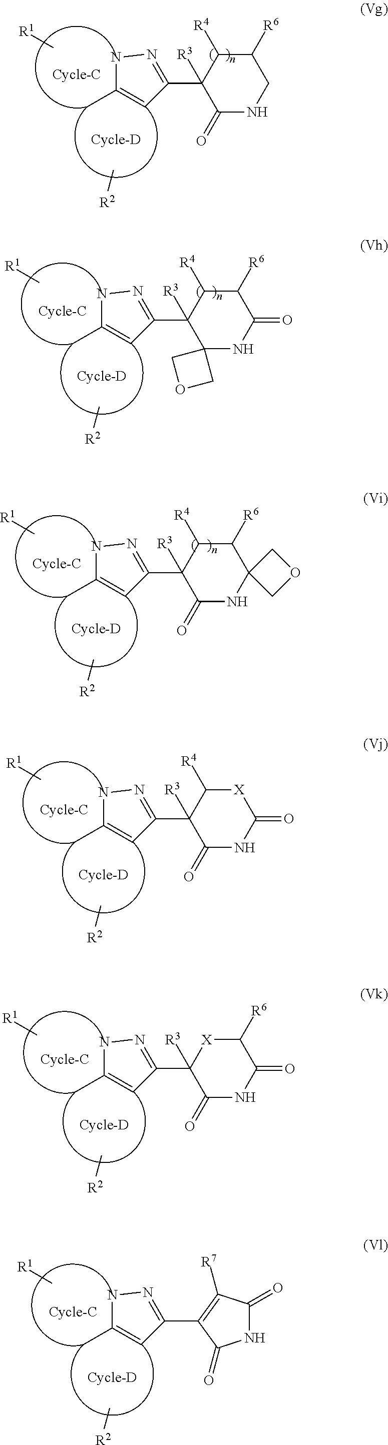

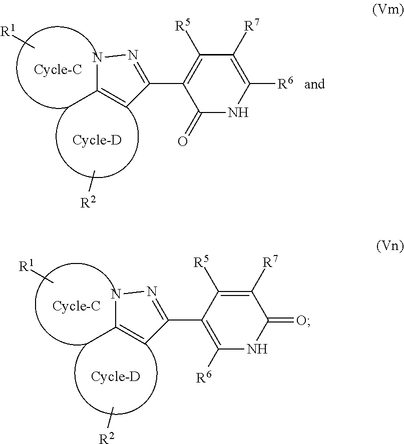

or a pharmaceutically acceptable salt thereof.

In certain embodiments, the compound of Formula (VI) is selected from Formula (VIa), Formula (VIb), Formula (VIc), Formula (VId), Formula (VIf), Formula (VIg), Formula (VIh), Formula (VIi), Formula (VIj), Formula (VIk), Formula (VIl), Formula (VIm), and Formula (VIn):

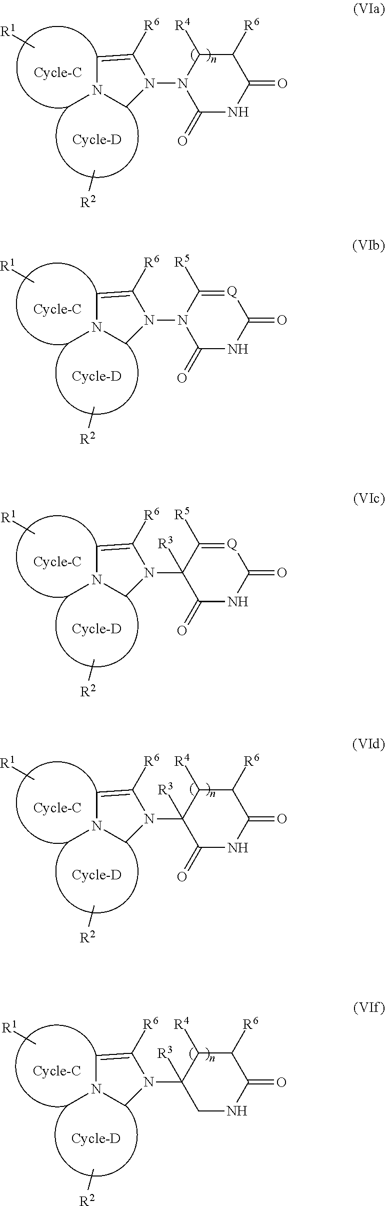

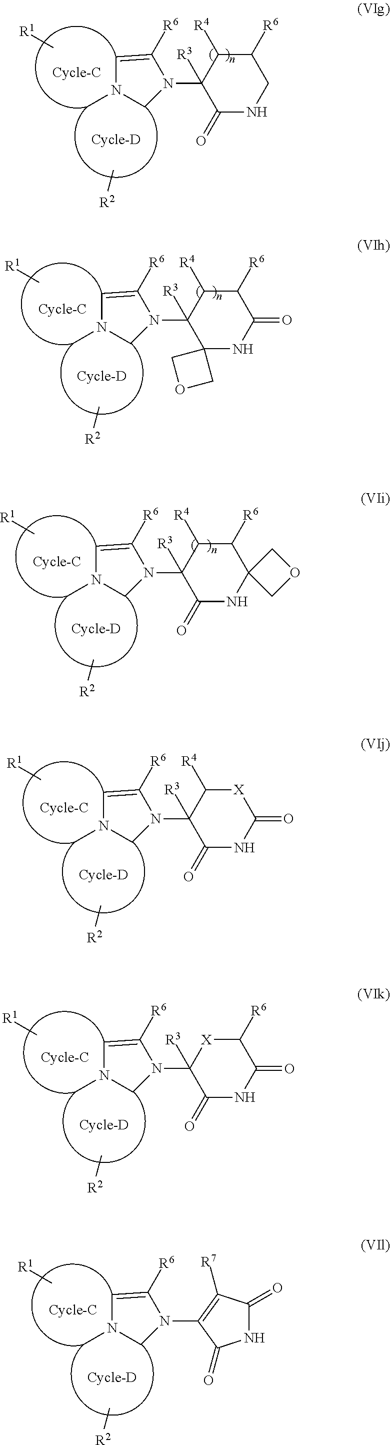

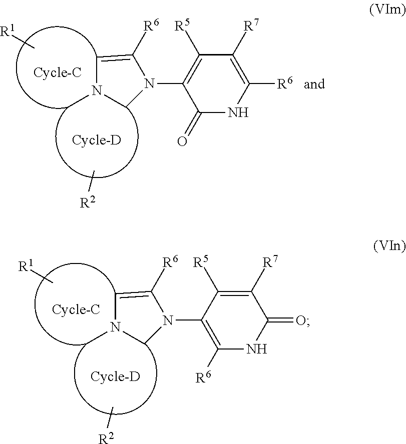

or a pharmaceutically acceptable salt thereof.

In certain embodiments, the compound of Formula (VII) is selected from Formula (VIIa), Formula (VIIb), Formula (VIIc), Formula (VIId), Formula (VIIe), Formula (VIIf), Formula (VIIg), Formula (VIIh), Formula (ViE), Formula (VIIj), Formula (VIIk), Formula (VIIl), Formula (VIIm), and Formula (VIIn):

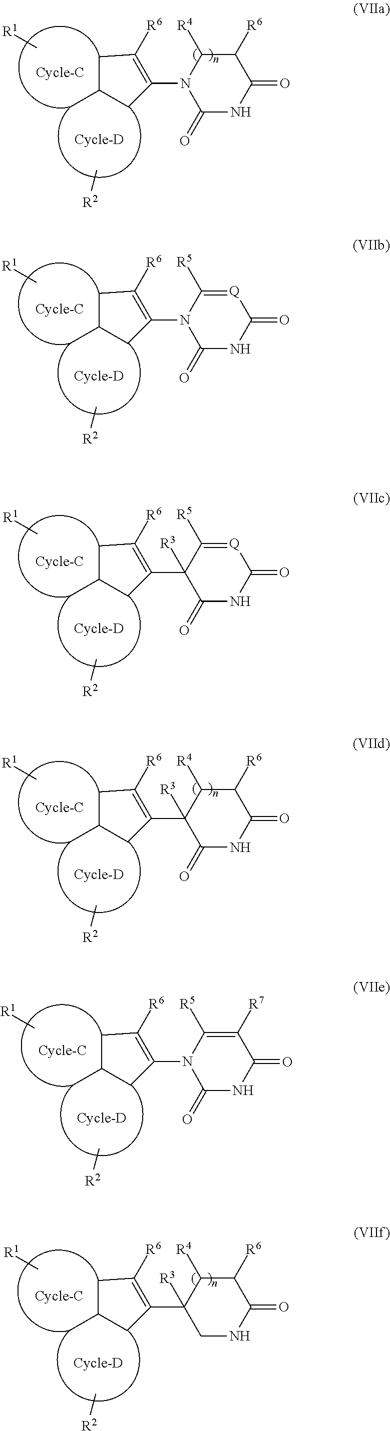

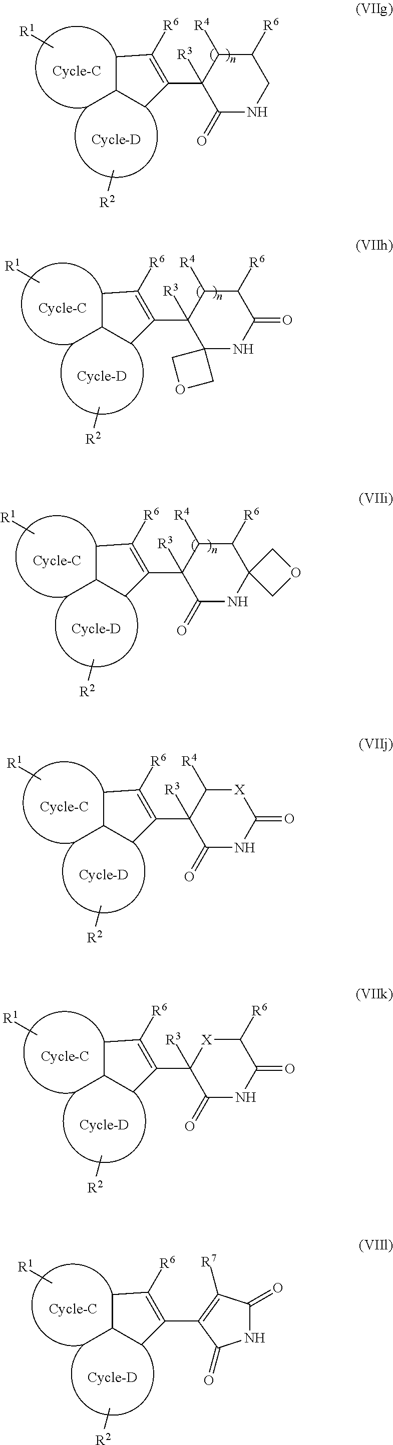

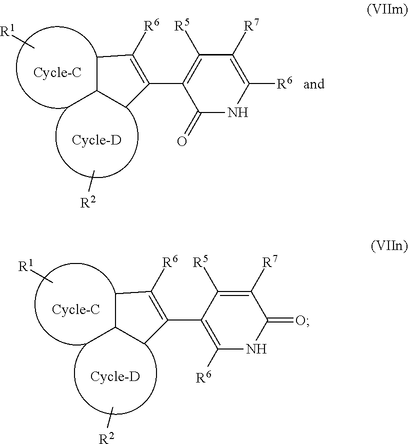

or a pharmaceutically acceptable salt thereof.

In certain embodiments, the compound of Formula (VIII) is selected from Formula (VIIIa), Formula (VIIIb), Formula (VIIIc), Formula (VIIId), Formula (VIIIe), Formula (VIIIf), Formula (VIIIg), Formula (VIIIh), Formula (VIIIi), Formula (VIIIj), Formula (VIIIk), Formula (VIIIl), Formula (VIIIm), and Formula (VIIIn):

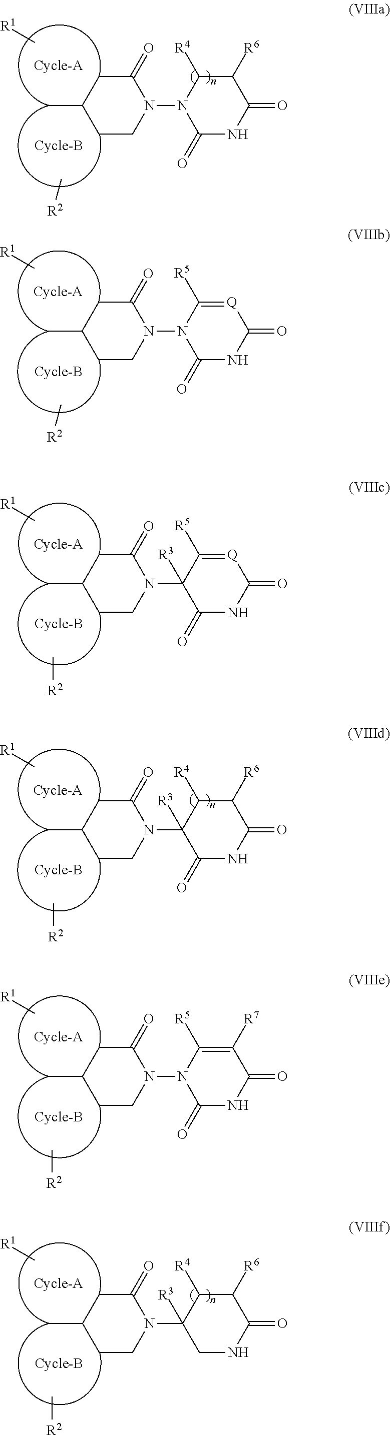

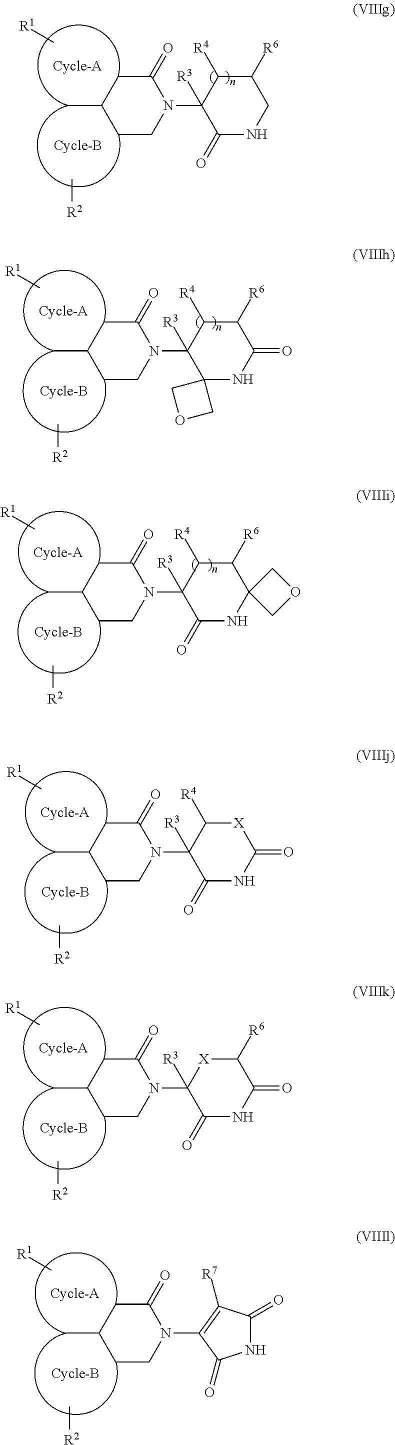

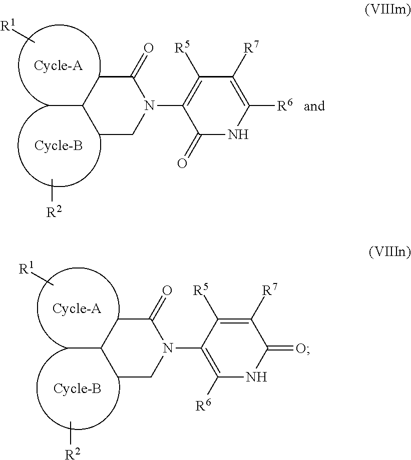

or a pharmaceutically acceptable salt thereof.

In certain embodiments, the compound of Formula (IX) is selected from Formula (IXa), Formula (IXb), Formula (IXc), Formula (IXd), Formula (IXe), Formula (IXf), Formula (IXg), Formula (IXh), Formula (IXi), Formula (IXj), Formula (IXk), Formula (IXl), Formula (IXm), and Formula (IXn):

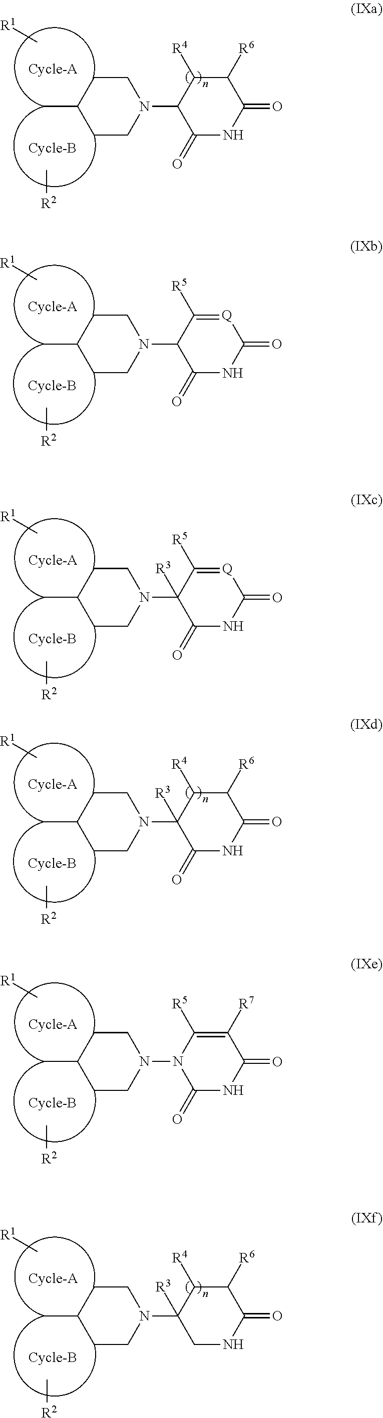

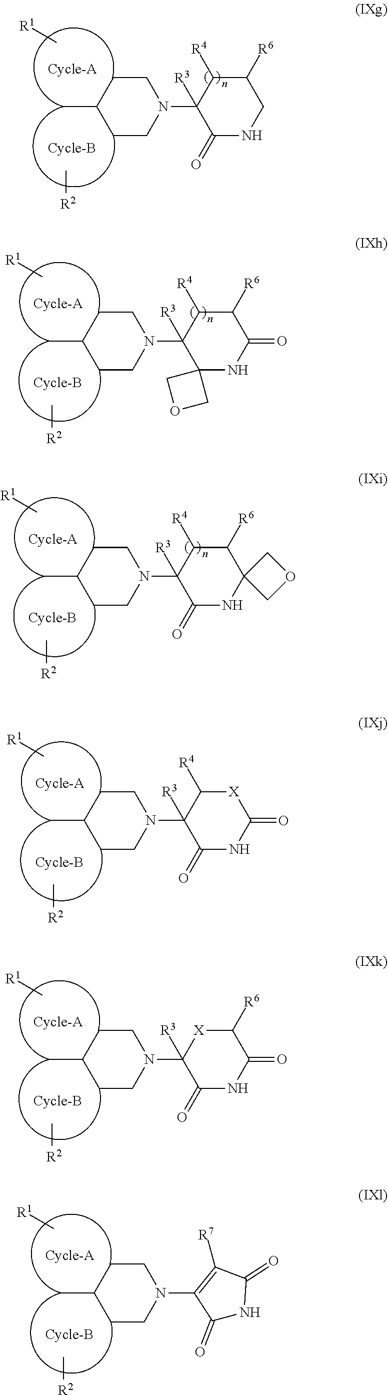

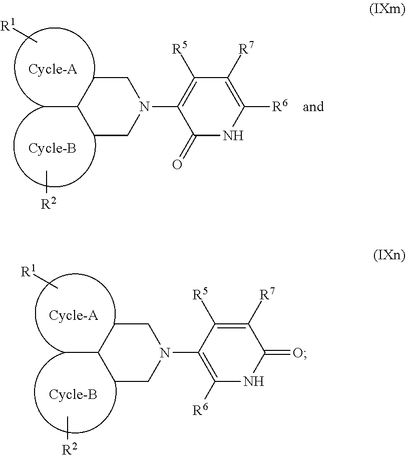

or a pharmaceutically acceptable salt thereof.

In certain embodiments, the compound of Formula (X) is selected from Formula (Xa), Formula (Xb), Formula (Xc), Formula (Xd), Formula (Xe), Formula (Xf), Formula (Xg), Formula (Xh), Formula (Xi), Formula (Xj), Formula (Xk), Formula (Xl), Formula (Xm), and Formula (Xn):

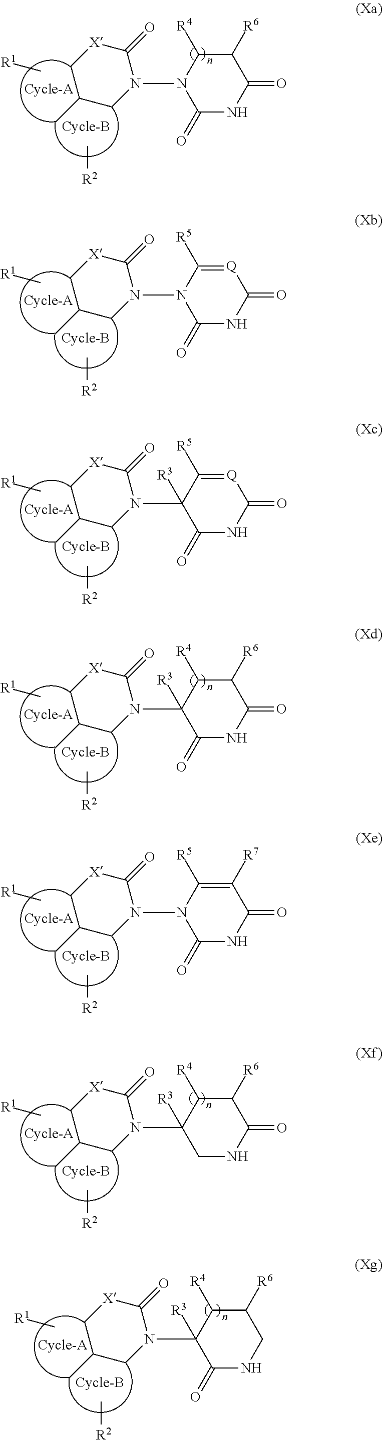

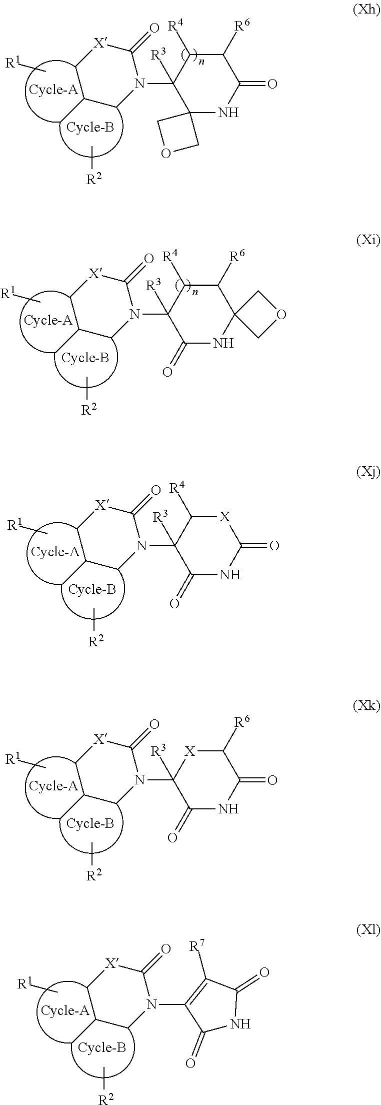

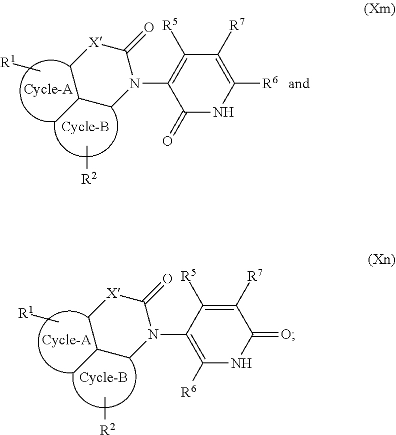

or a pharmaceutically acceptable salt thereof.

In certain embodiments, the compound of Formula (XI) is selected from Formula (XIa), Formula (XIb), Formula (XIc), Formula (XId), Formula (XIe), Formula (XIf), Formula (XIg), Formula (XIh), Formula (XIi), Formula (XIj), Formula (XIl), Formula (XIm), and Formula (XIn):

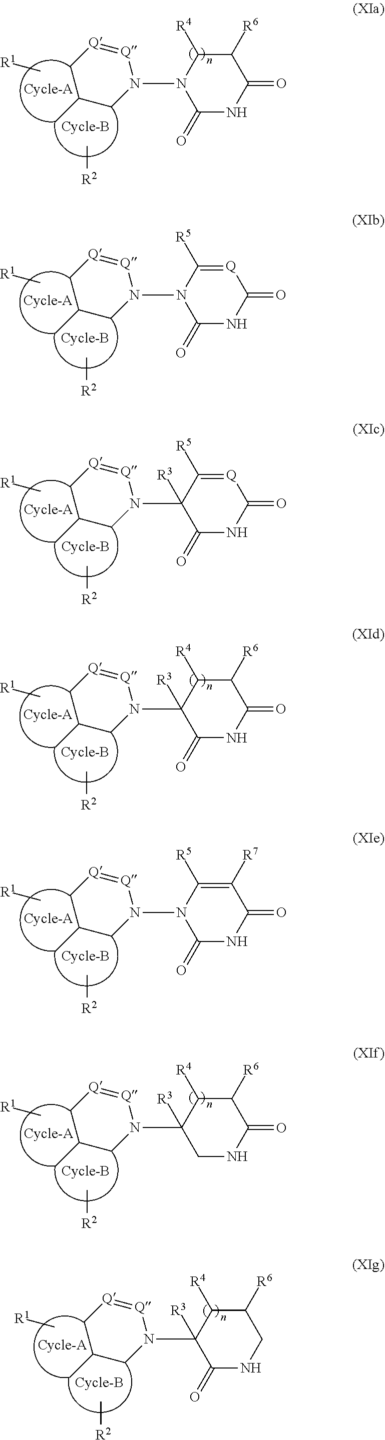

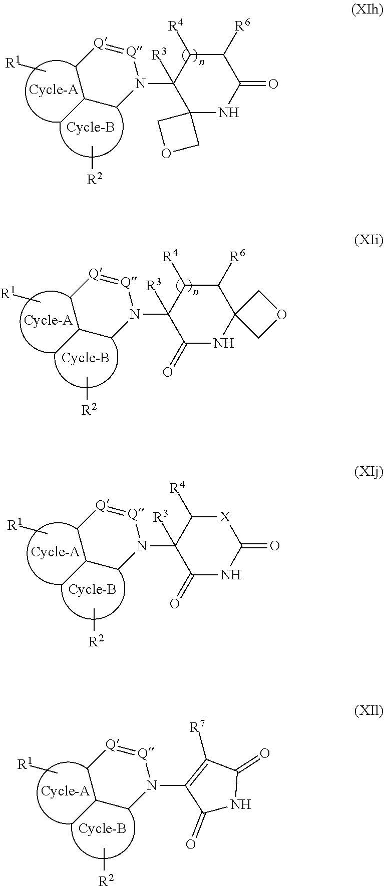

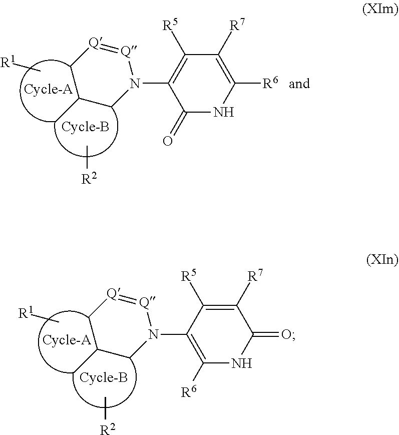

or a pharmaceutically acceptable salt thereof.

In certain embodiments, the compound of Formula (XII) is selected from Formula (XIIa), Formula (XIIb), Formula (XIIc), Formula (XIId), Formula (XIIe), Formula (XIIf), Formula (XIIg), Formula (XIIh), Formula (XIi), Formula (XIIj), Formula (XIIk), Formula (XIIl), Formula (XIIm), and Formula (XIIn):

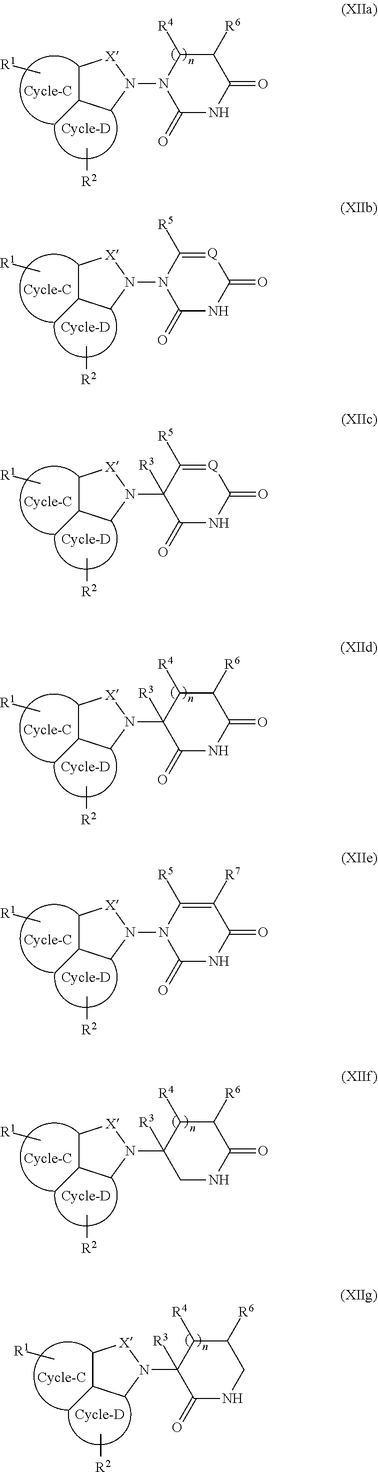

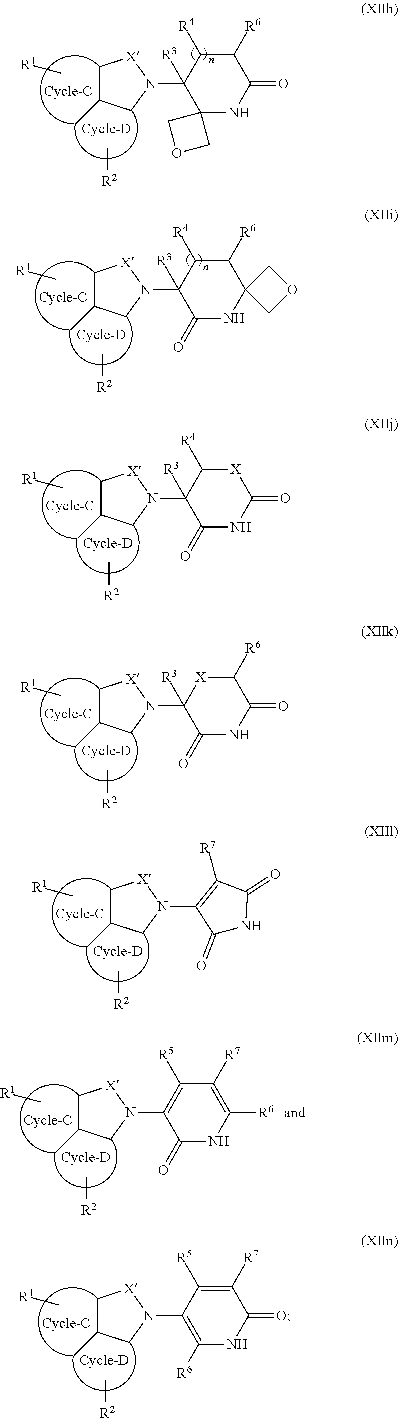

or a pharmaceutically acceptable salt thereof.

In certain embodiments, the compound of Formula (XIII) is selected from Formula (XIIIa), Formula (XIIIb), Formula (XIIIc), Formula (XIIId), Formula (XIIIe), Formula (XIIIf), Formula (XIIIg), Formula (XIIIh), Formula (XIIIi), Formula (XIIIj), Formula (XIIIk), Formula (XIIIl), Formula (XIIIm), and Formula (XIIIn):

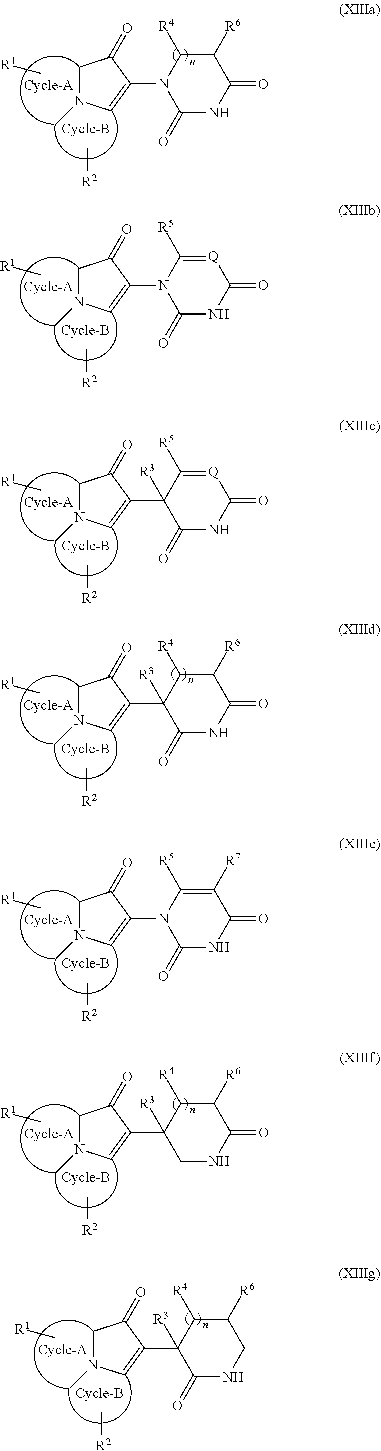

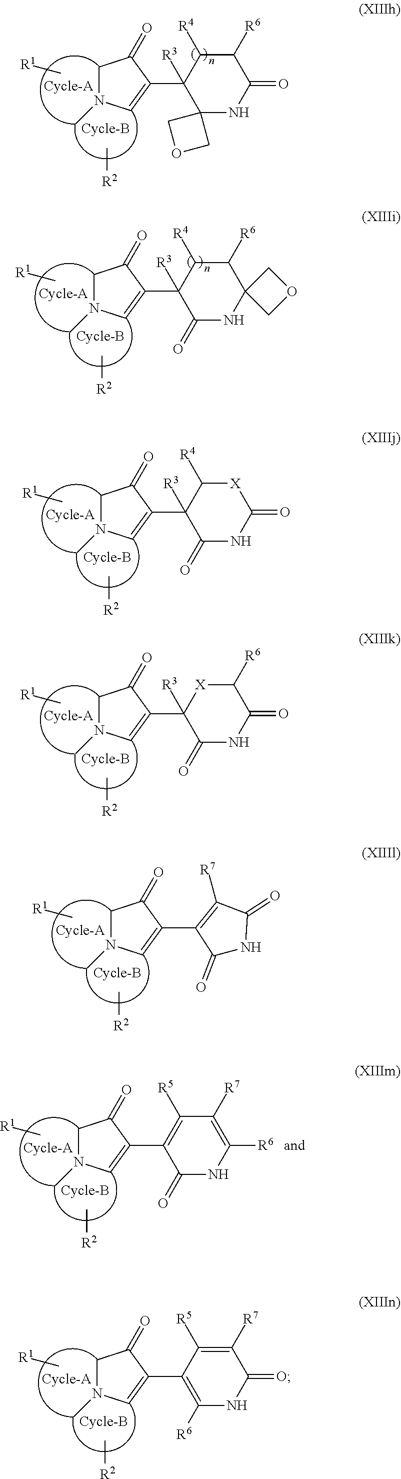

or a pharmaceutically acceptable salt thereof.

In certain embodiments, the compound of Formula (XIV) is selected from Formula (XIVa), Formula (XIVb), Formula (XIVc), Formula (XIVd), Formula (XIVe), Formula (XIVf), Formula (XIVg), Formula (XIVh), Formula (XIVi), Formula (XIVj), Formula (XIVk), Formula (XIVI), Formula (XIVm), and Formula (XIVn):

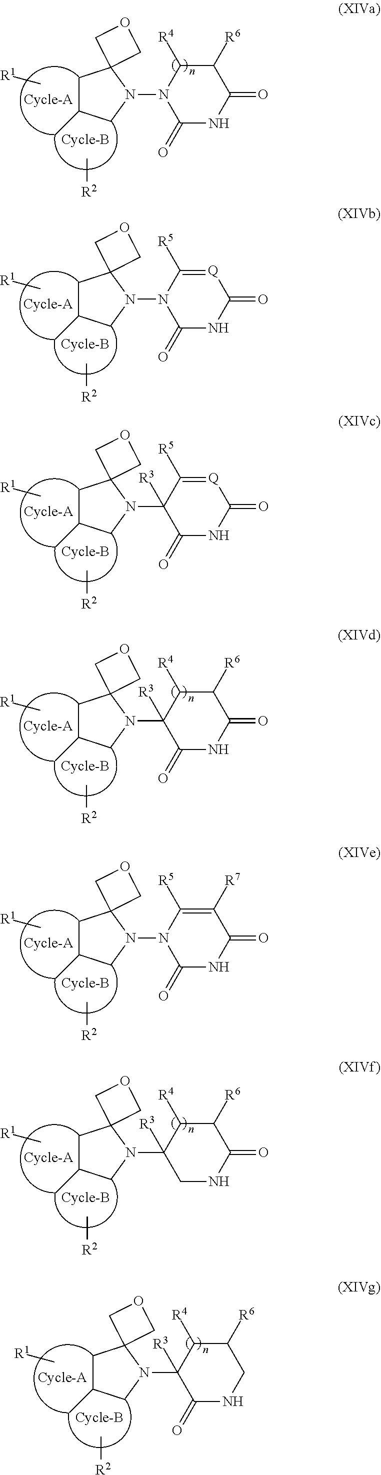

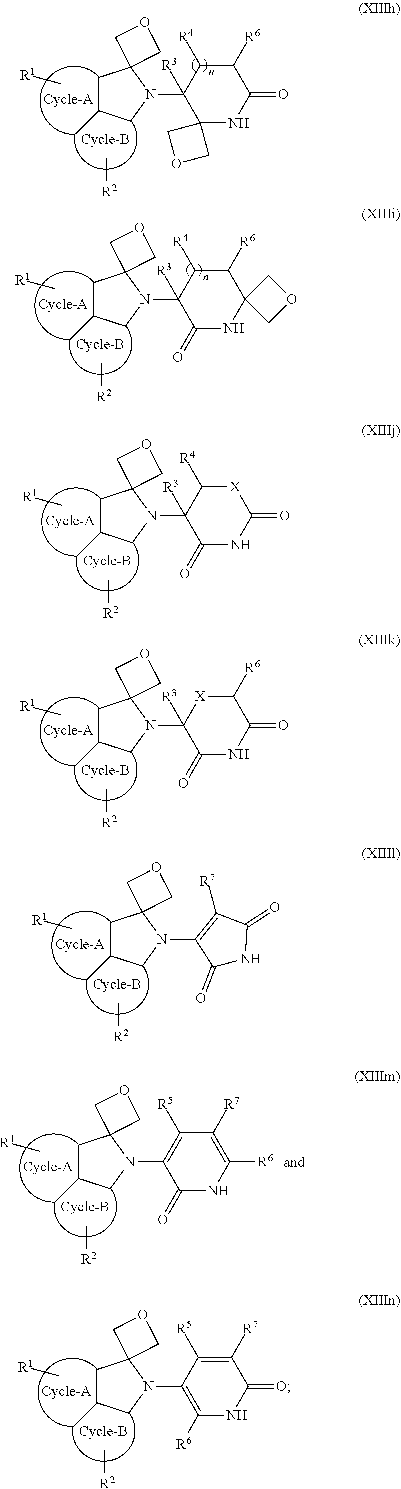

or a pharmaceutically acceptable salt thereof.

In certain embodiments, the compound of Formula (XVII) is selected from Formula (XVIIa), Formula (XVIIb), Formula (XVIIc), Formula (XVIId), Formula (XVIIe), Formula (XVIIf), Formula (XVIIg), Formula (XVIIh), Formula (XVIIi), Formula (XVIIj), Formula (XVIIk), Formula (XVIIl), Formula (XVIIm), and Formula (XVIIn):

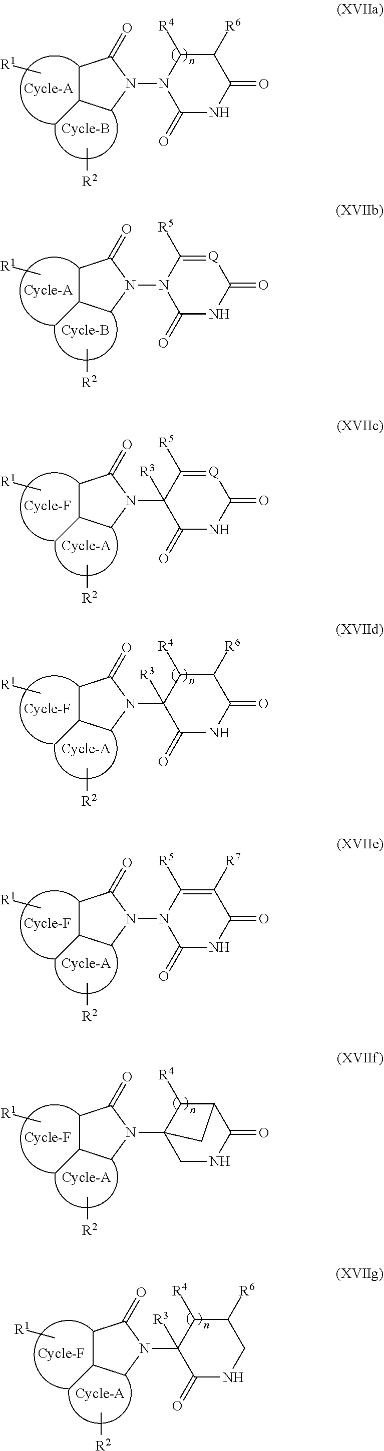

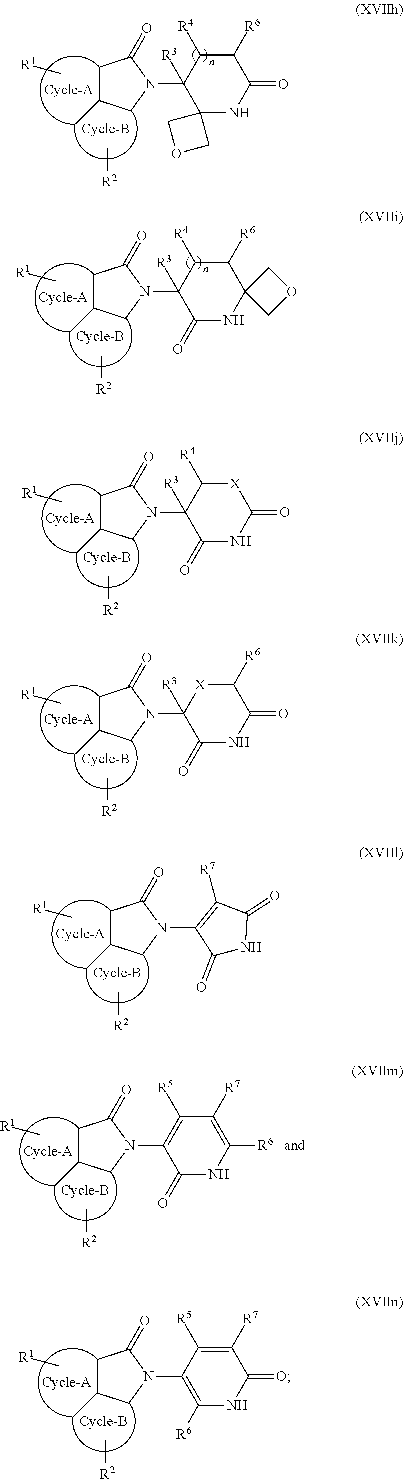

or a pharmaceutically acceptable salt thereof.

Non-limiting Examples of compounds of Formula I include:

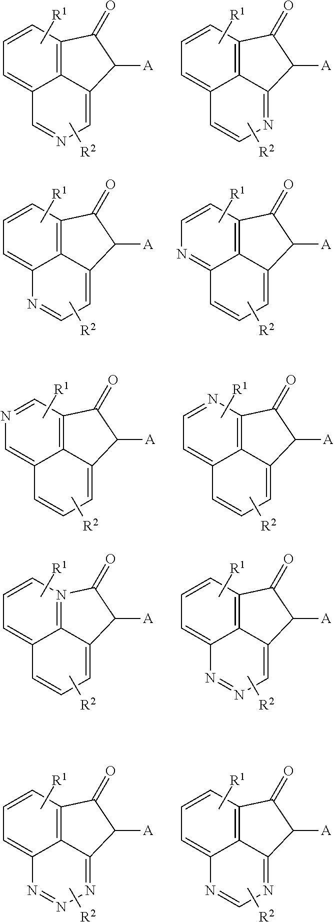

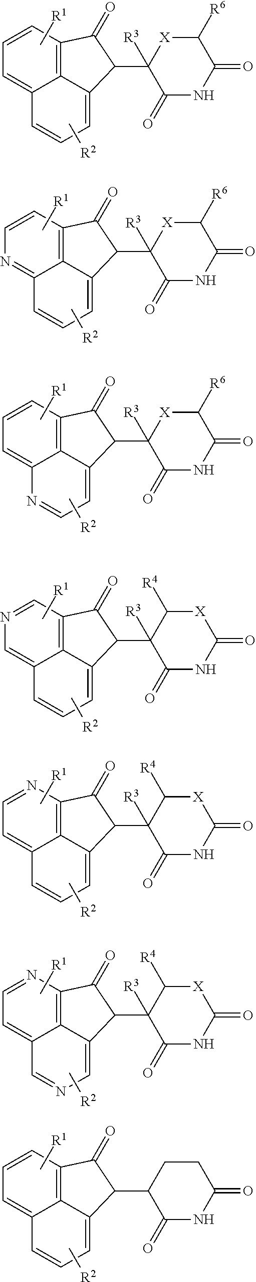

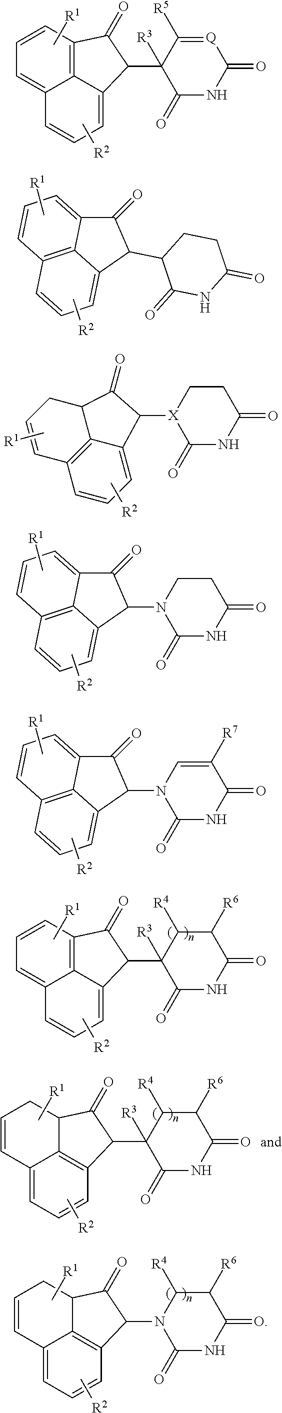

Non-limiting Examples of compounds of Formula II include:

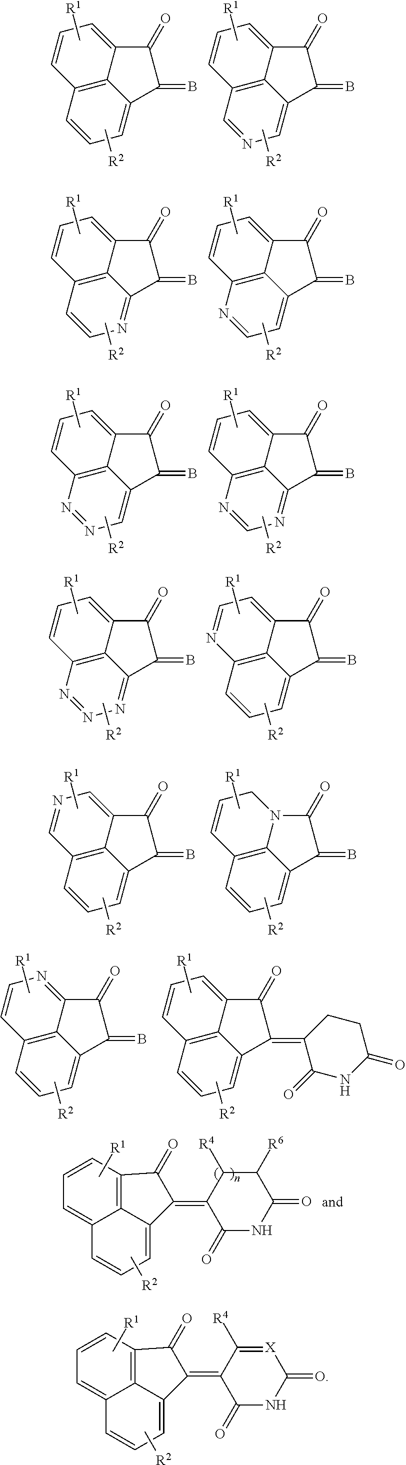

Non-limiting Examples of compounds of Formula III include:

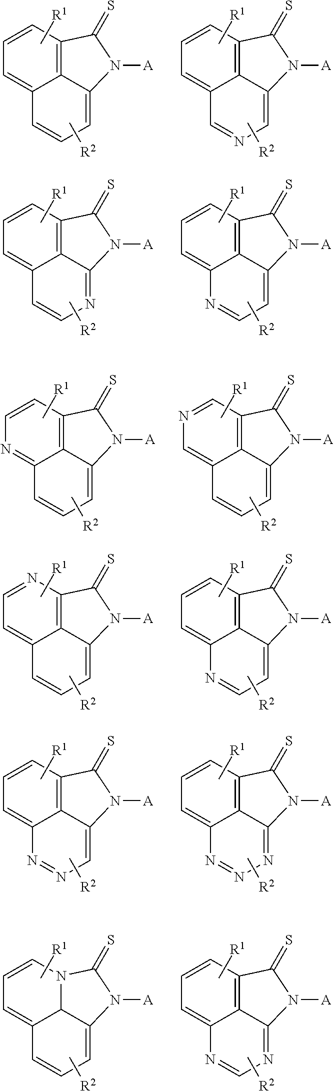

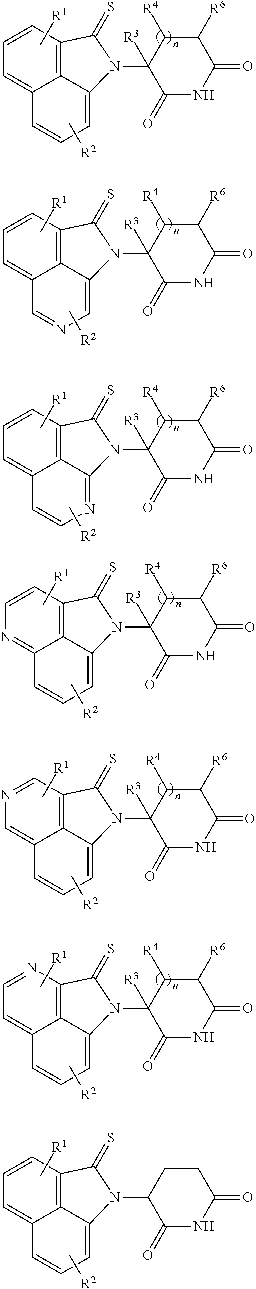

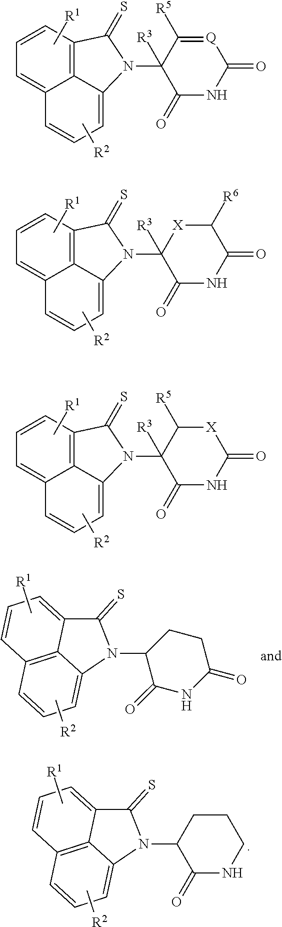

Non-limiting Examples of compounds of Formula IV include:

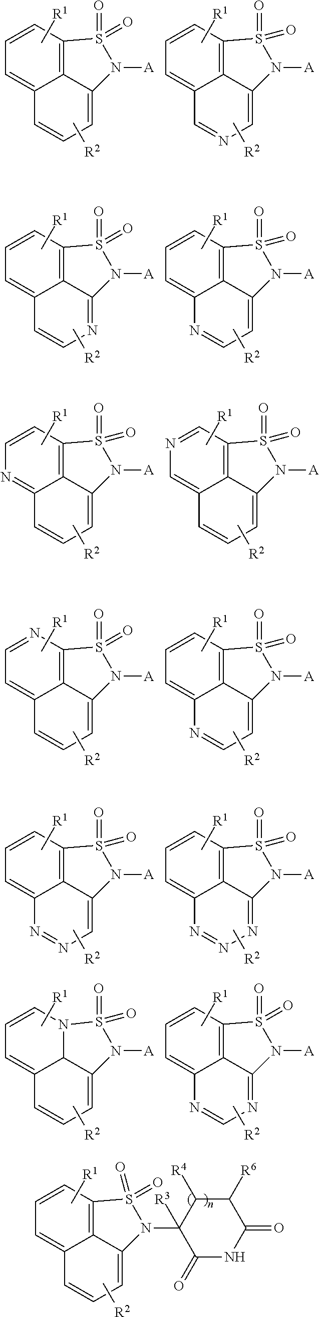

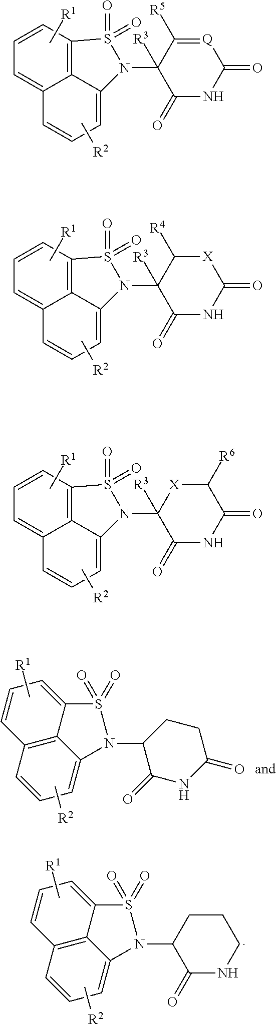

Non-limiting Examples of compounds of Formula V include:

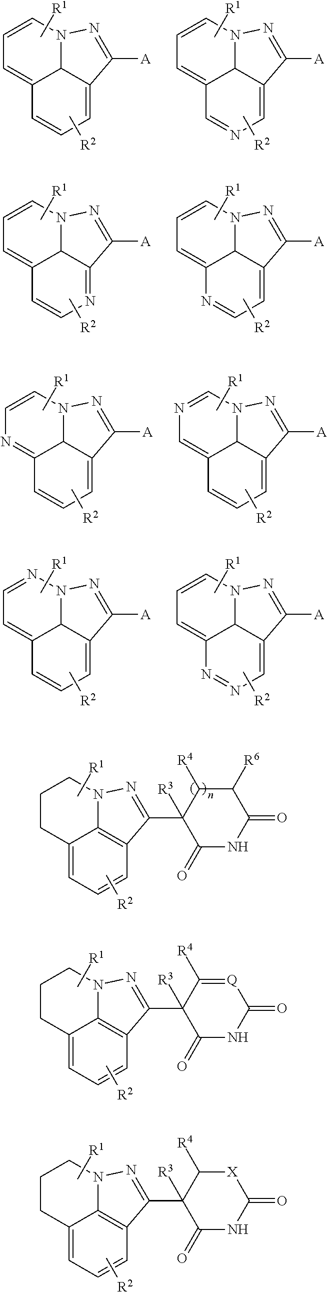

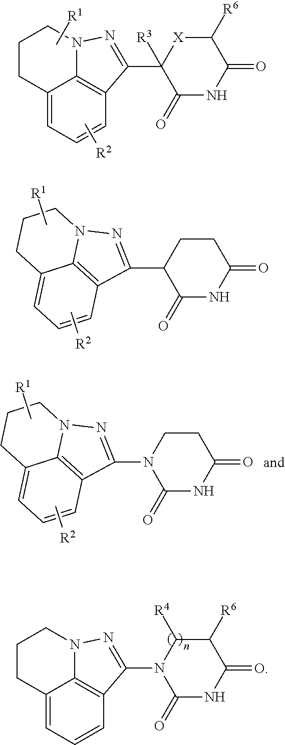

Non-limiting Examples of compounds of Formula VI include:

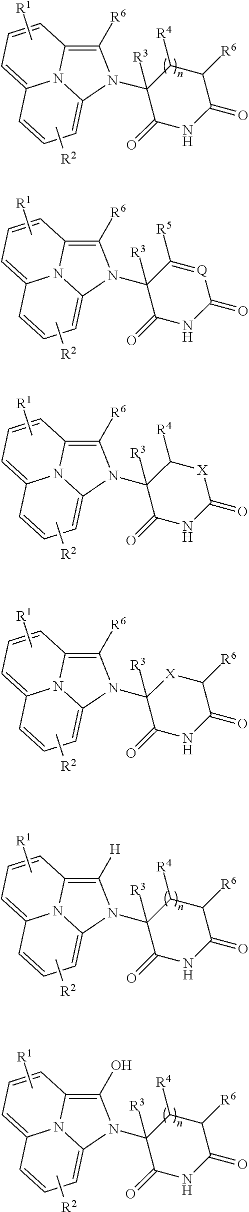

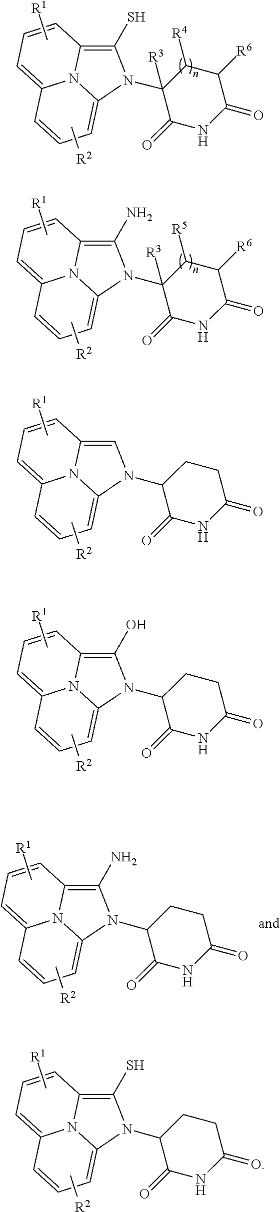

Non-limiting Examples of compounds of Formula VII include:

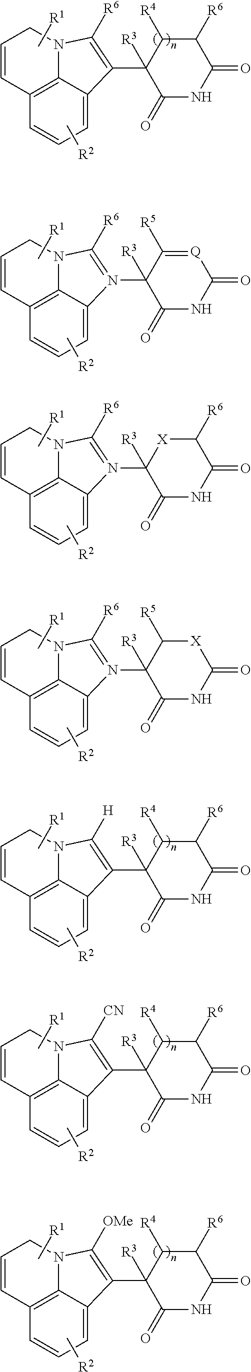

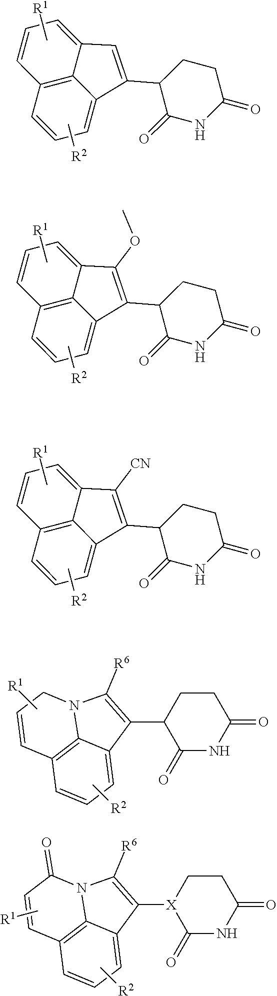

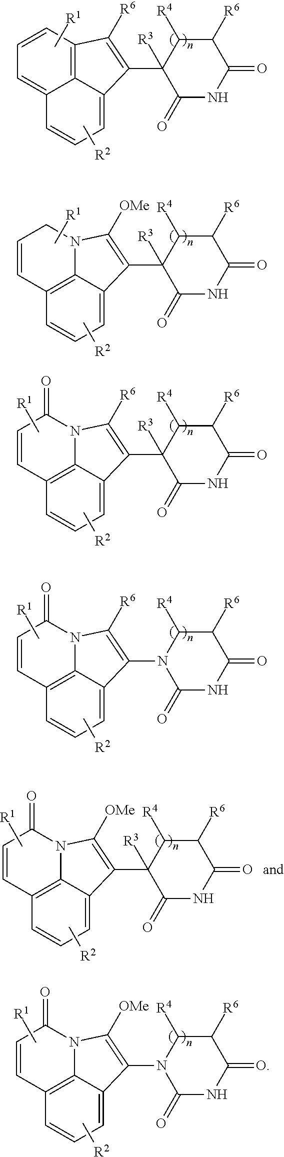

Non-limiting Examples of compounds of Formula VIII include:

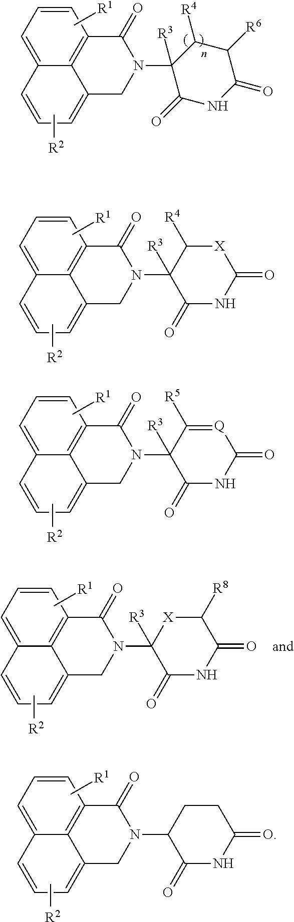

Non-limiting Examples of compounds of Formula IX include:

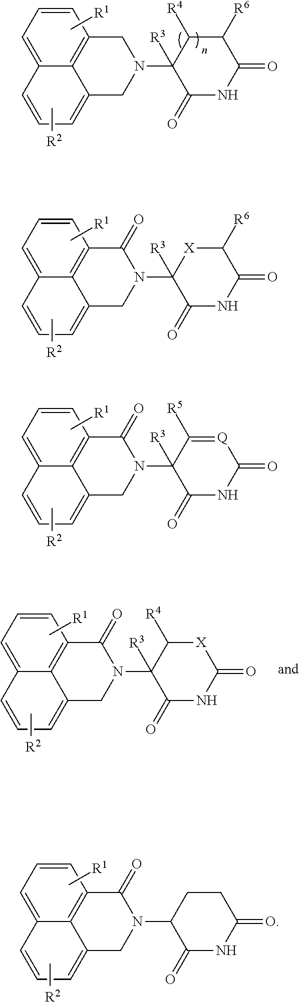

Non-limiting Examples of compounds of Formula X include:

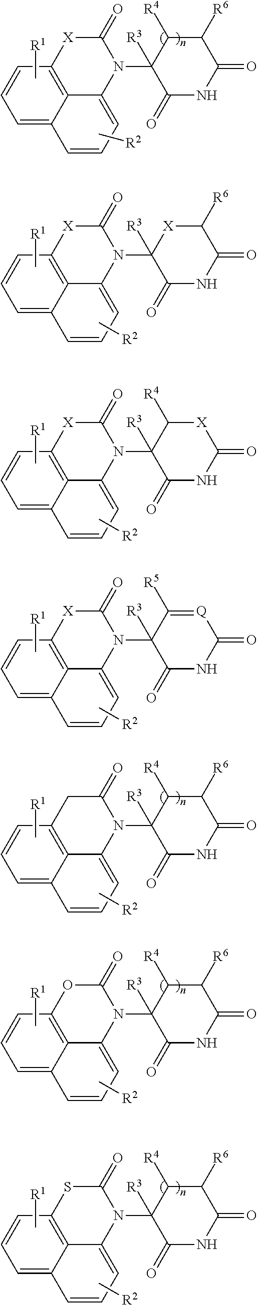

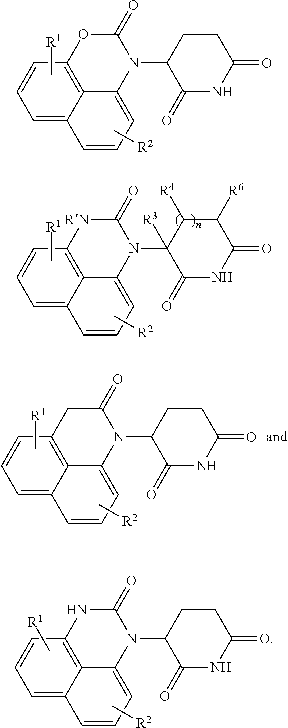

Non-limiting Examples of compounds of Formula XI include:

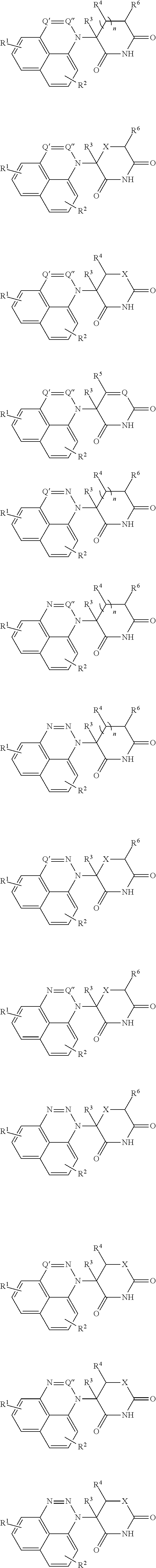

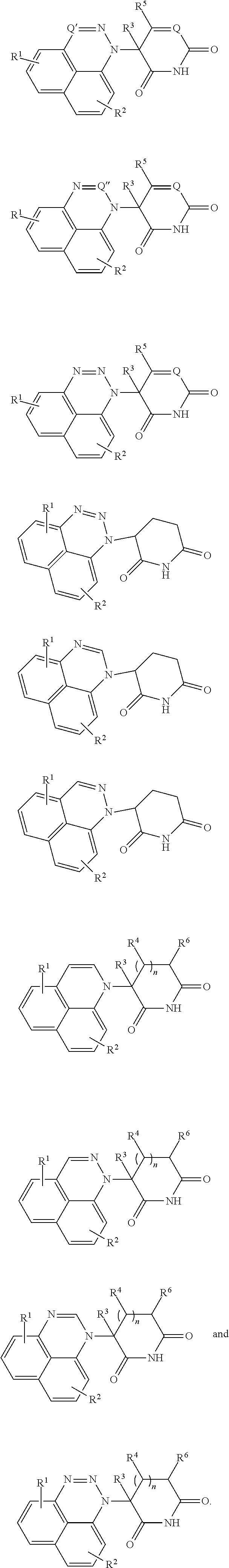

Non-limiting Examples of compounds of Formula XII include:

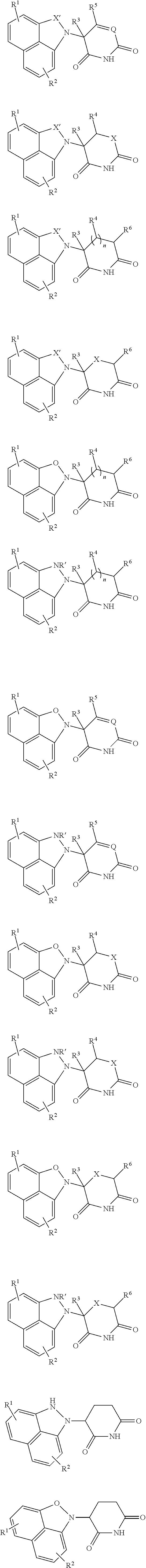

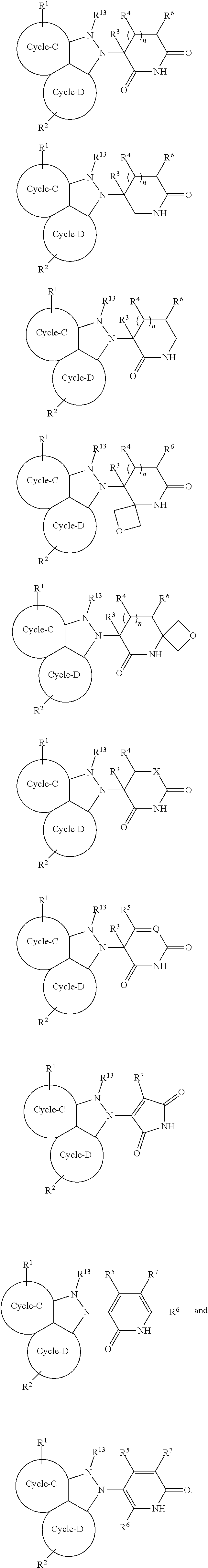

Non-limiting Examples of compounds of Formula XIII include:

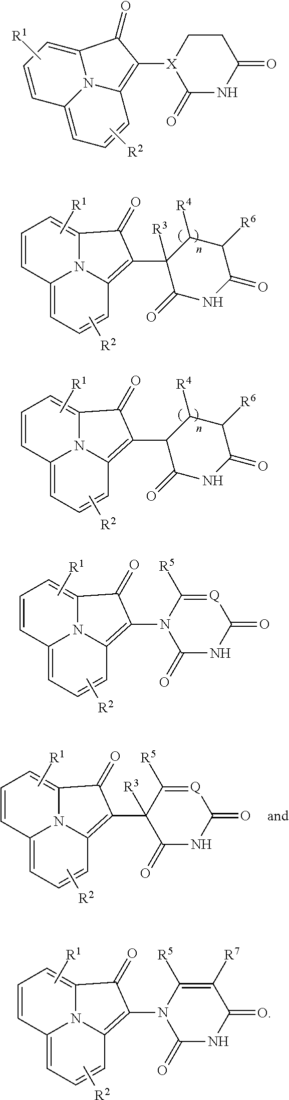

Non-limiting Examples of compounds of Formula XIV include:

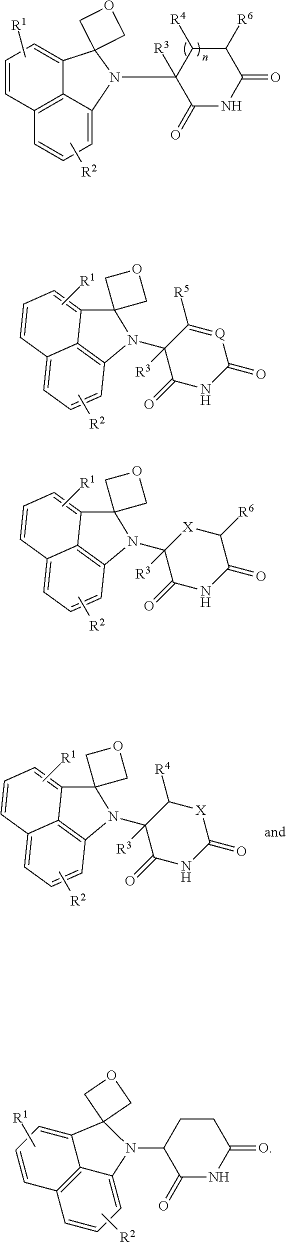

Non-limiting Examples of compounds of Formula XV include:

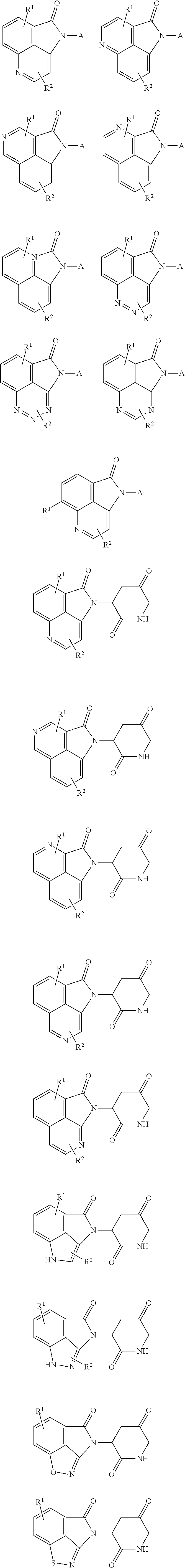

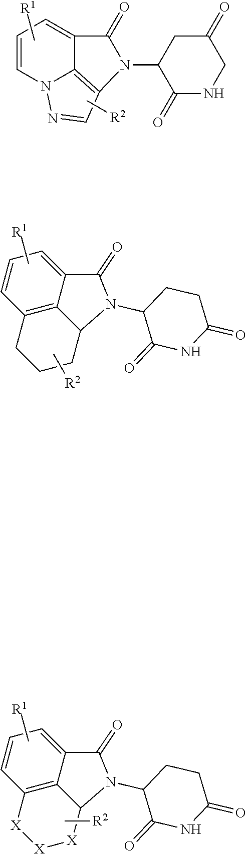

Additional non-limiting Examples of compounds of Formula XV include:

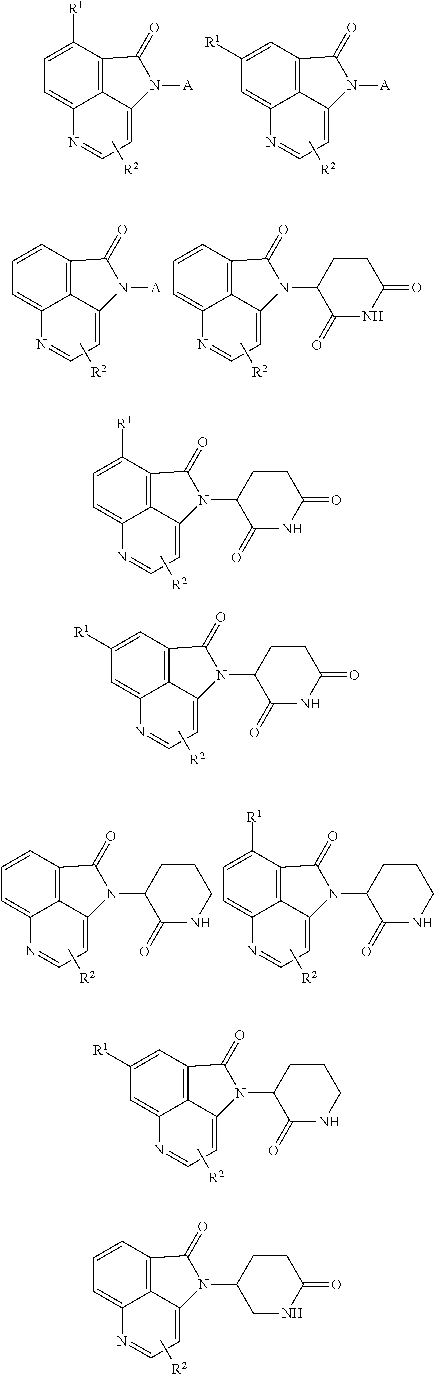

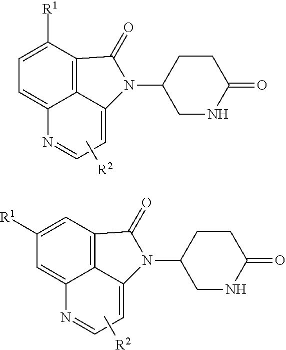

Additional non-limiting Examples of compounds of Formula XV include:

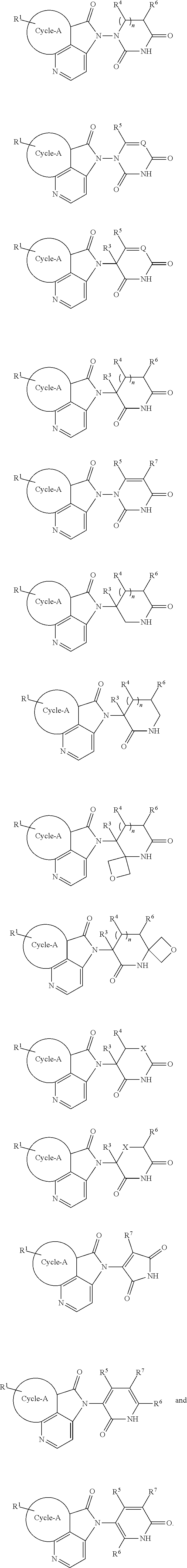

Additional non-limiting Examples of compounds of Formula XV include:

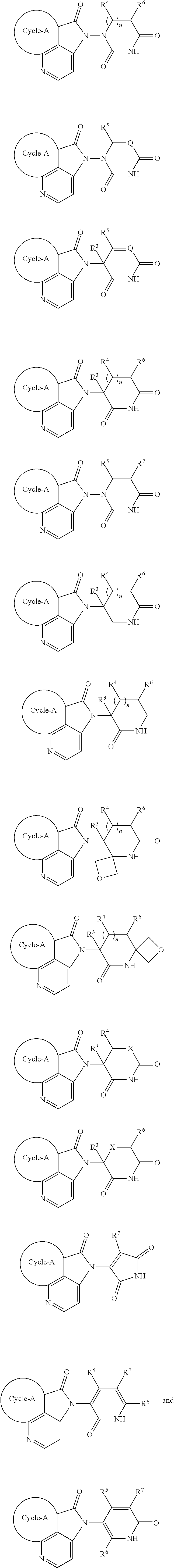

Non-limiting Examples of compounds of Formula XVI include:

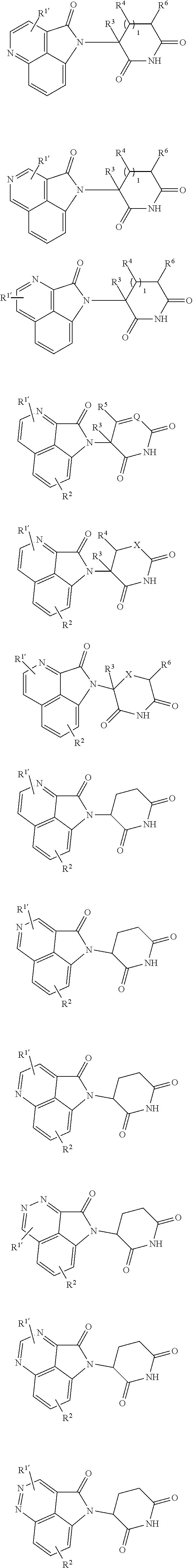

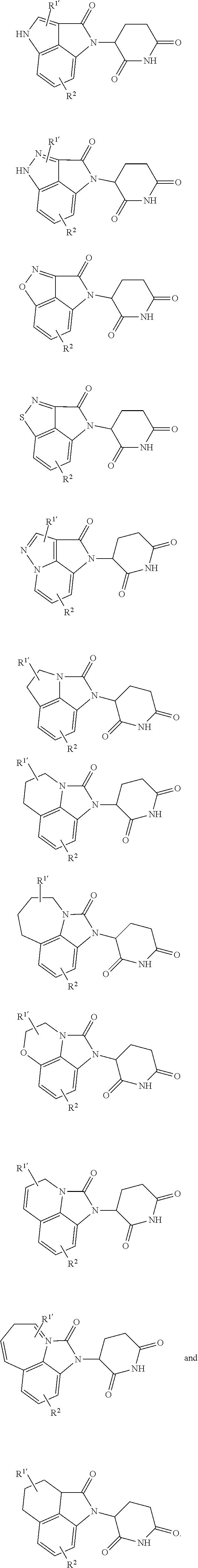

Additional non-limiting Examples of compounds of Formula XVI include:

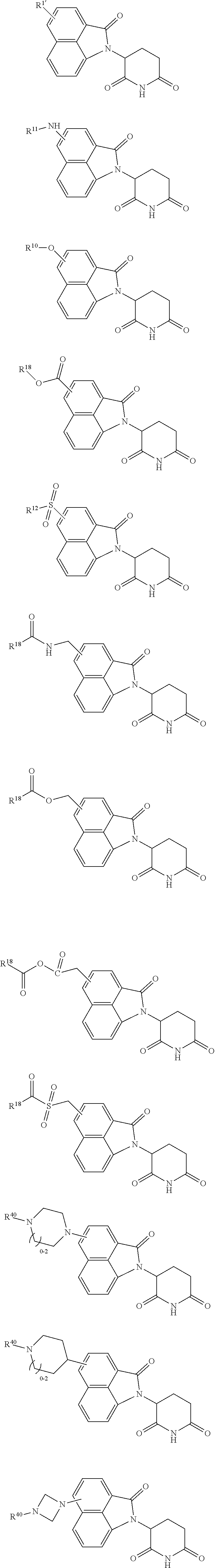

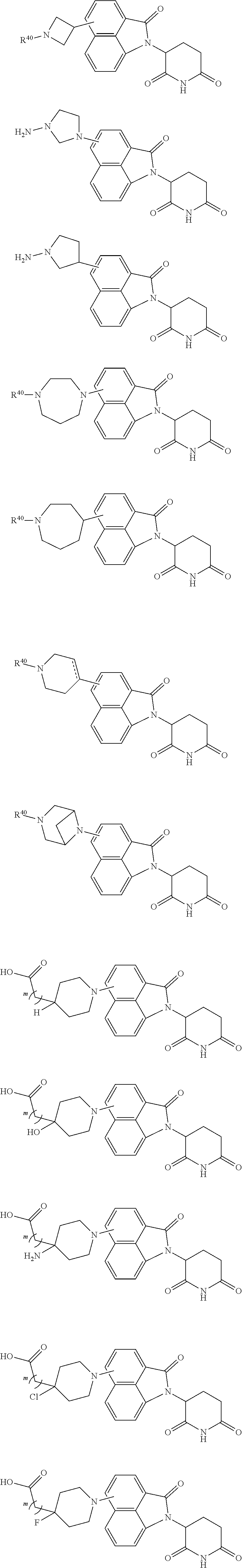

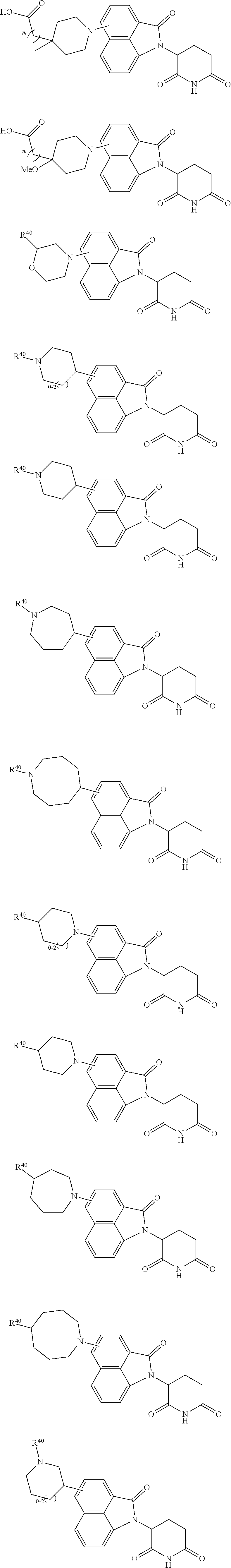

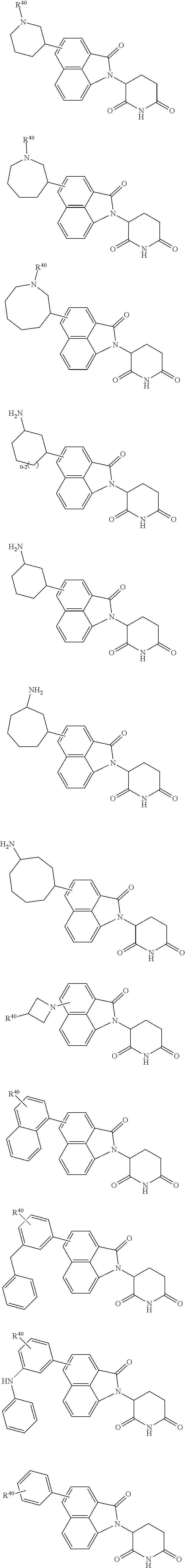

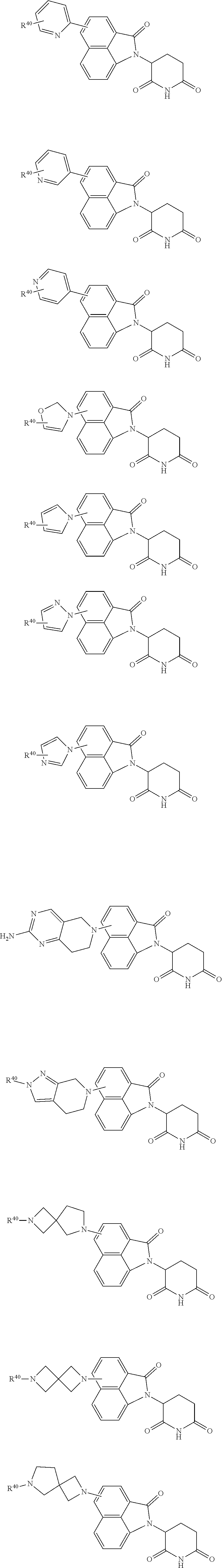

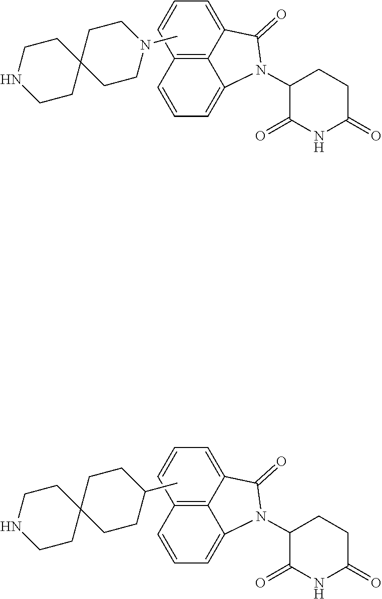

wherein m is an integer selected from 0, 1, 2, 3, 4, or 5.

Additional non-limiting Examples of compounds of Formula XVI include:

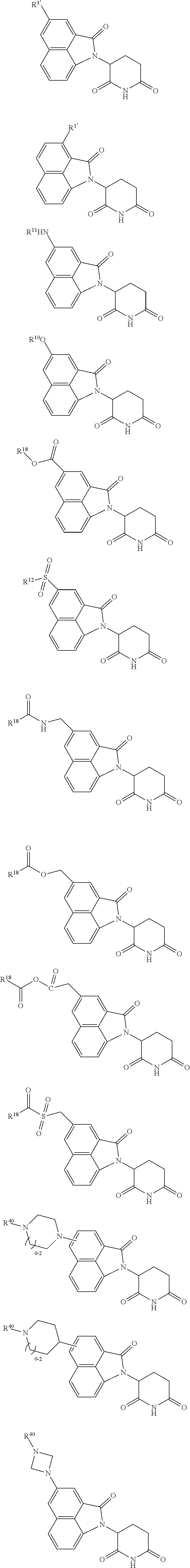

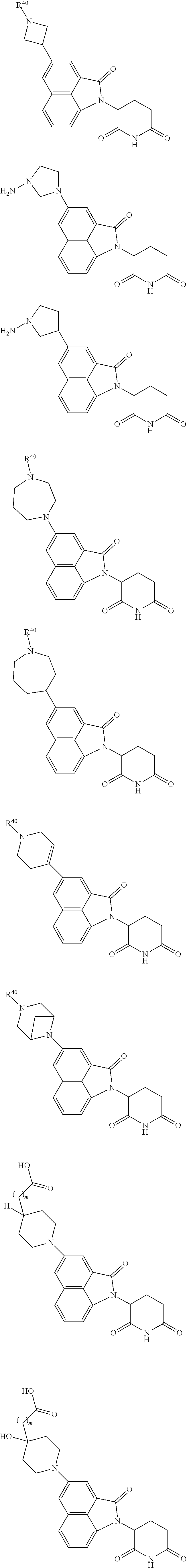

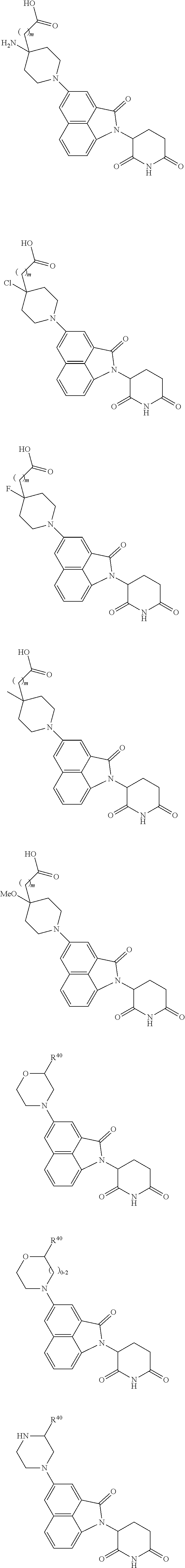

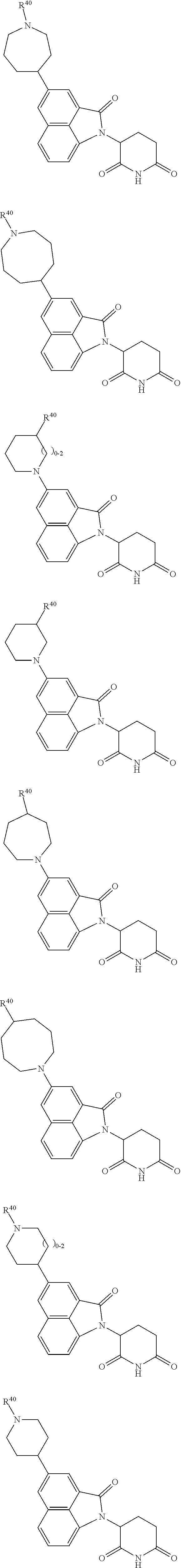

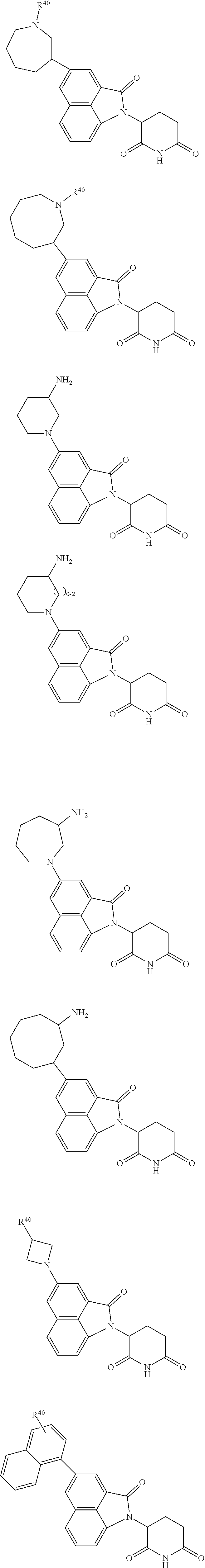

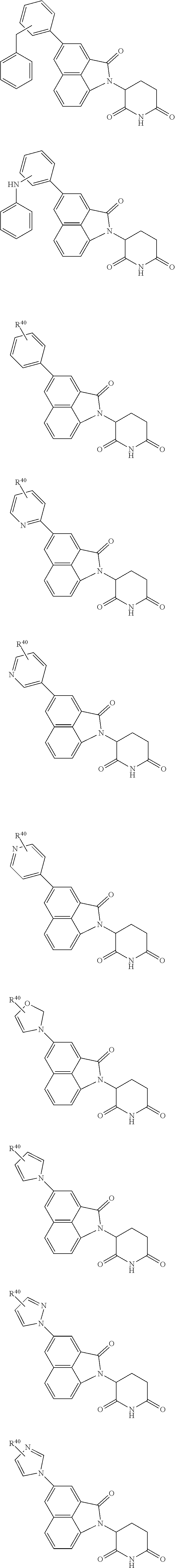

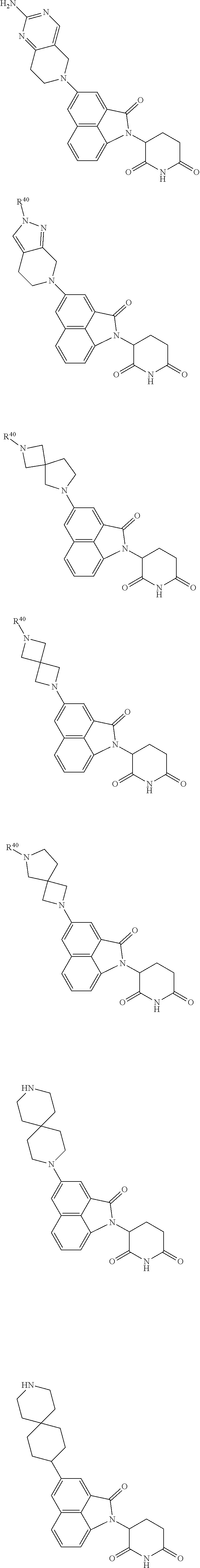

wherein m is an integer selected from 0, 1, 2, 3, 4, or 5.

Non-limiting Examples of compounds of Formula XVII include:

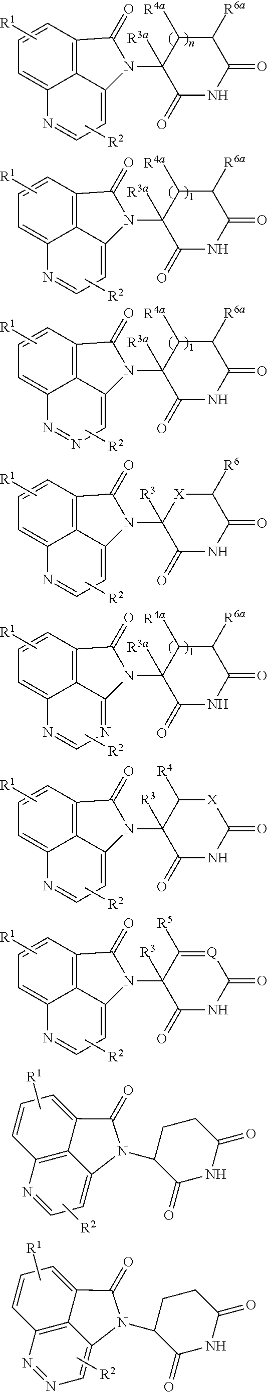

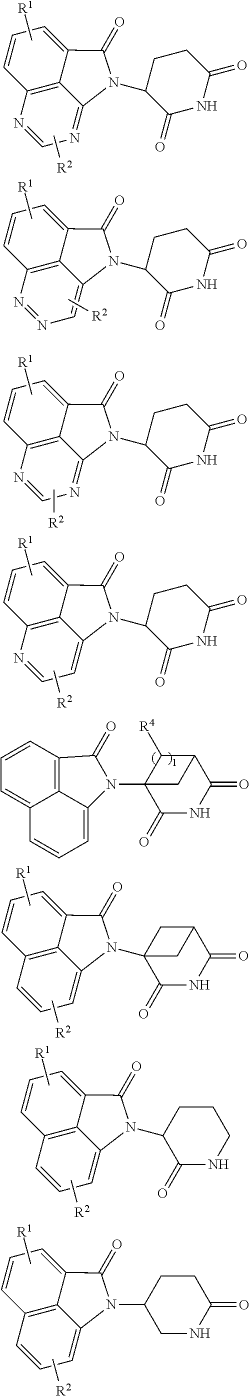

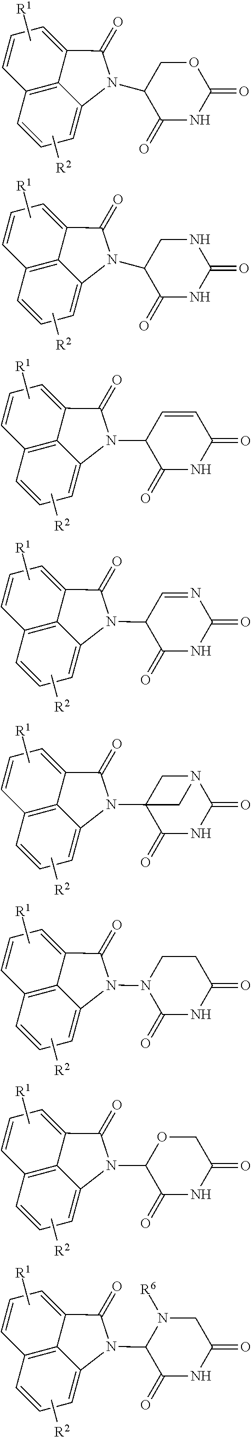

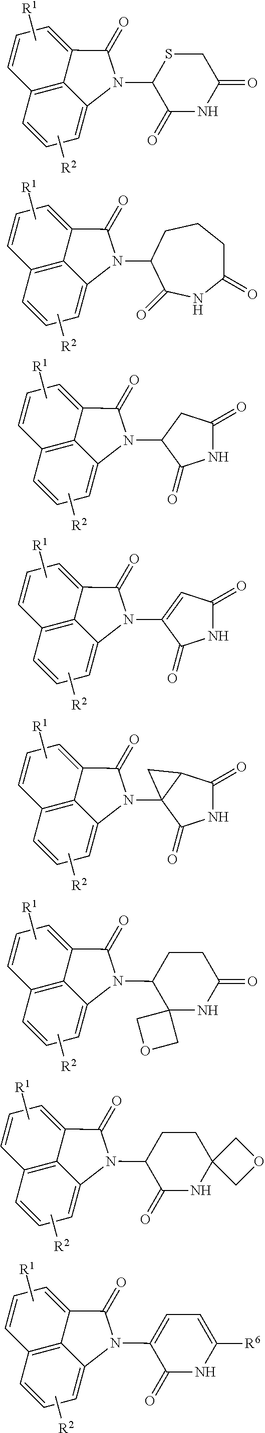

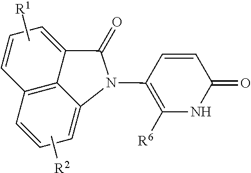

Additional non-limiting Examples of compounds of Formula XVII include:

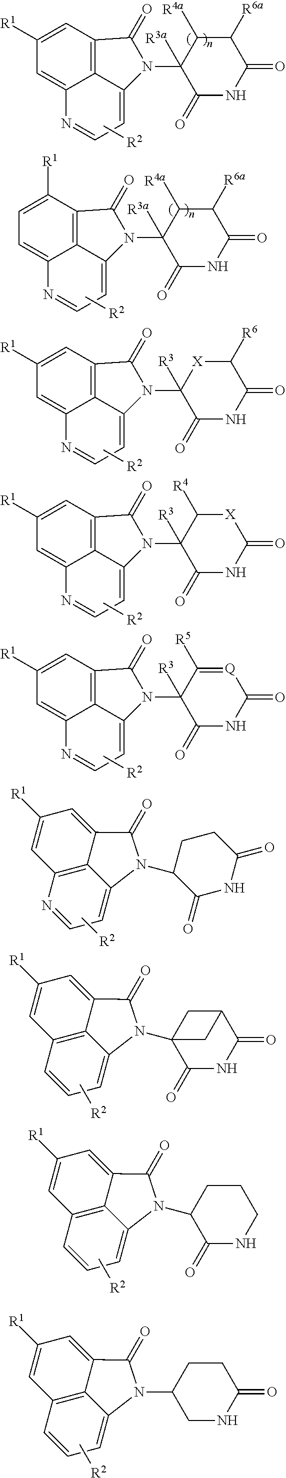

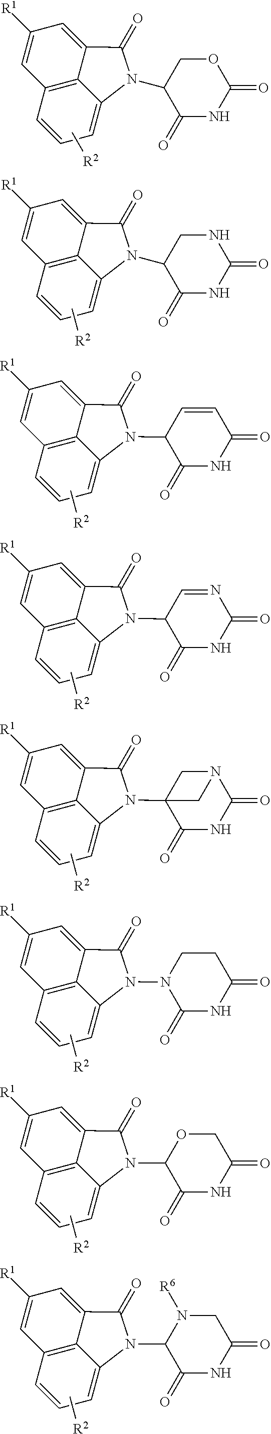

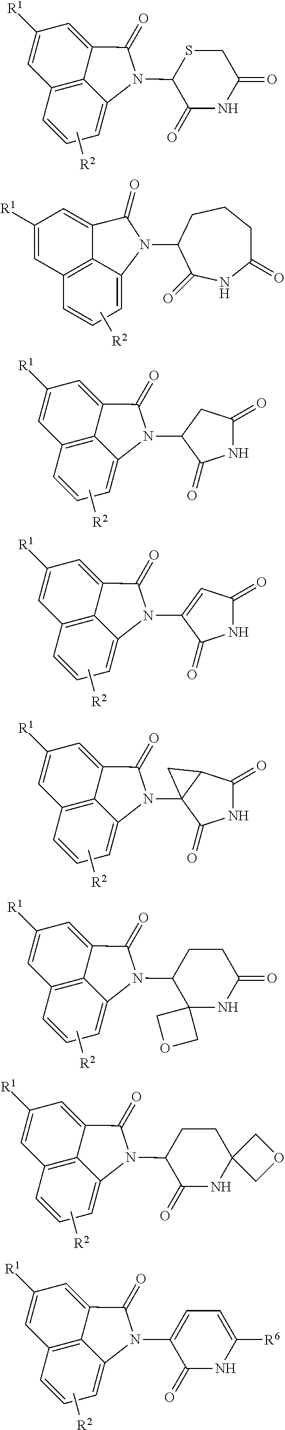

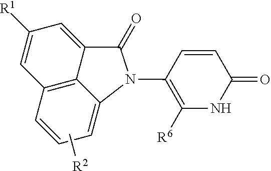

In the structures herein, a hydroxyl (for example an R¹ or R² group) is positioned on a heteroaryl ring carbon adjacent to a nitrogen, only one tautomer is shown as a shorthand method of referring individually to each separate tautomer or a mixture thereof, unless otherwise indicated herein, and each separate tautomer or mixture thereof is incorporated into the specification as if it were individually recited herein. This is demonstrated by the non-limiting examples of:

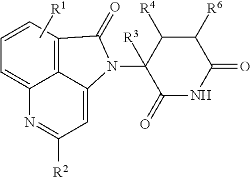

which includes both

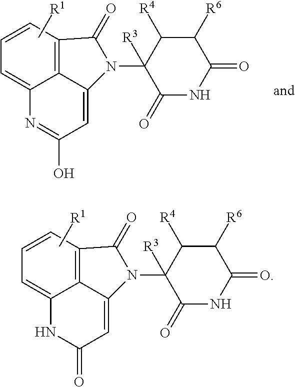

When bonds are depicted with brackets, this represents that the bond can be located at any position allowed by valence and stability. As a non-limiting example to illustrate the meaning of the brackets the following bracketed compound

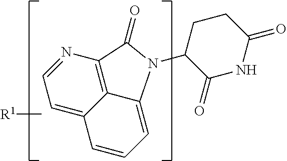

includes

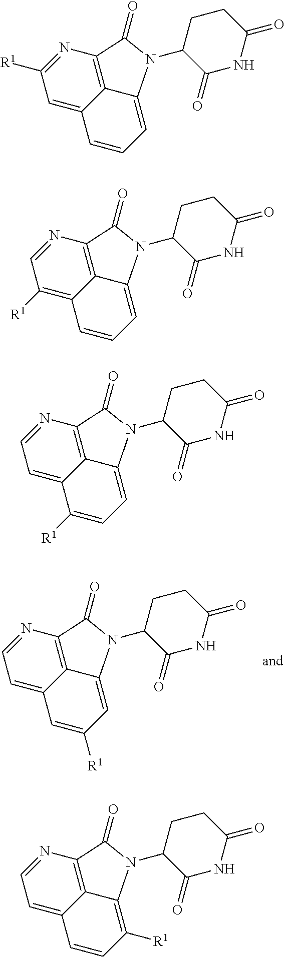

independently as if separately drawn.

Non-limiting examples of compounds of the present invention include:

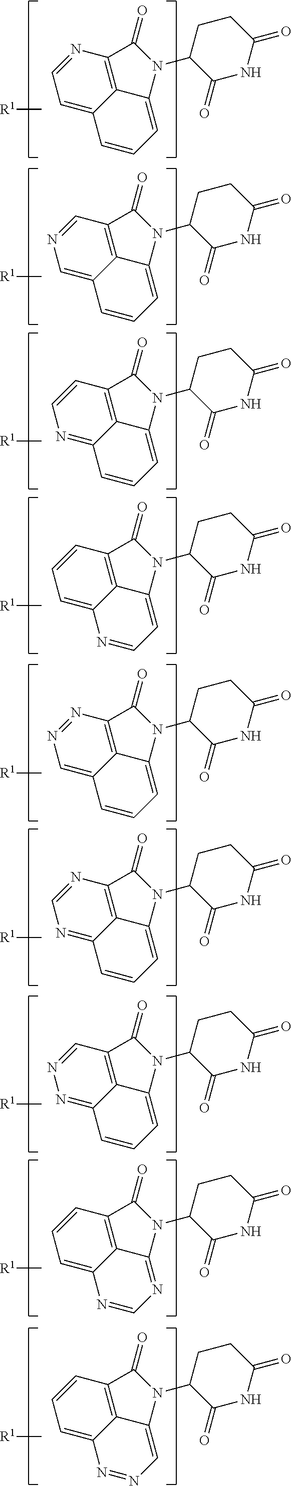

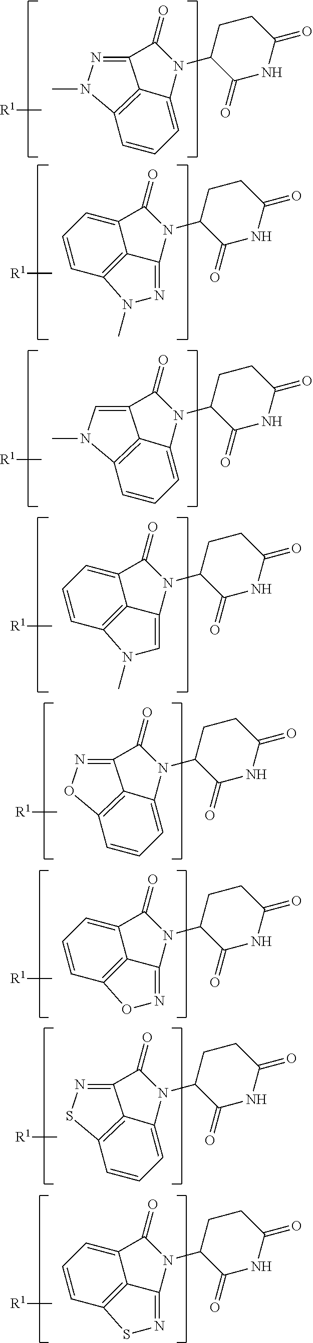

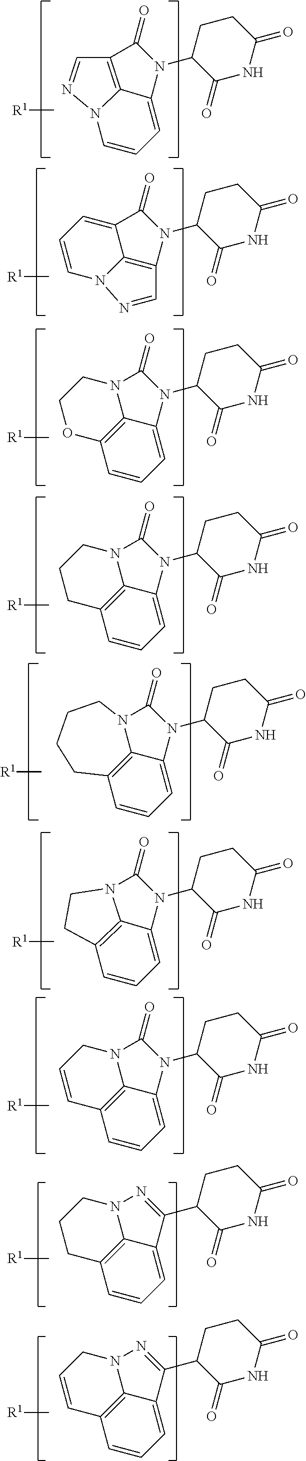

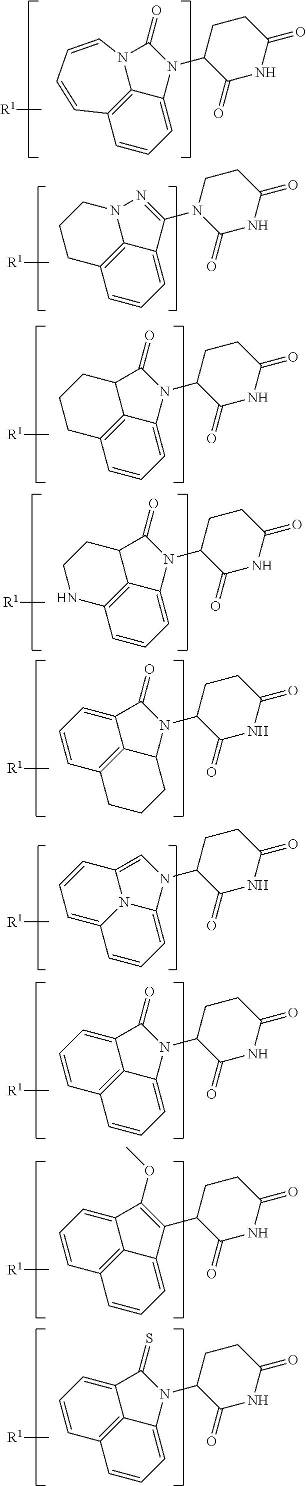

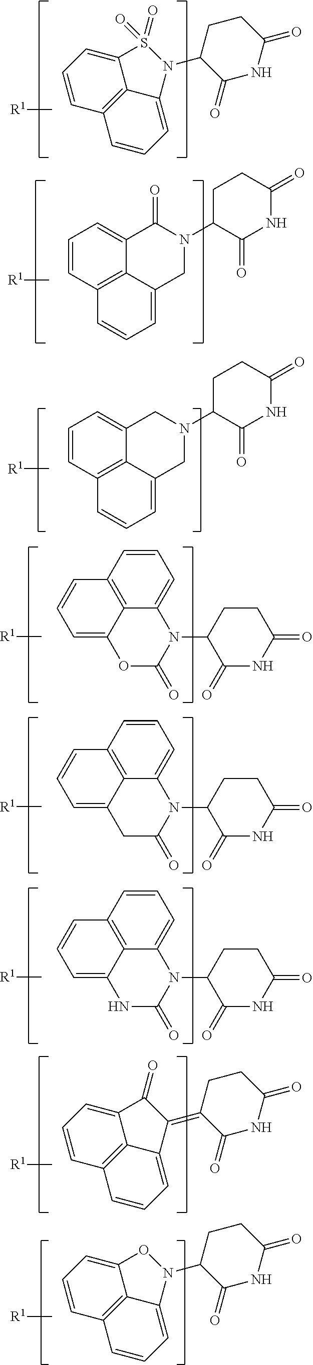

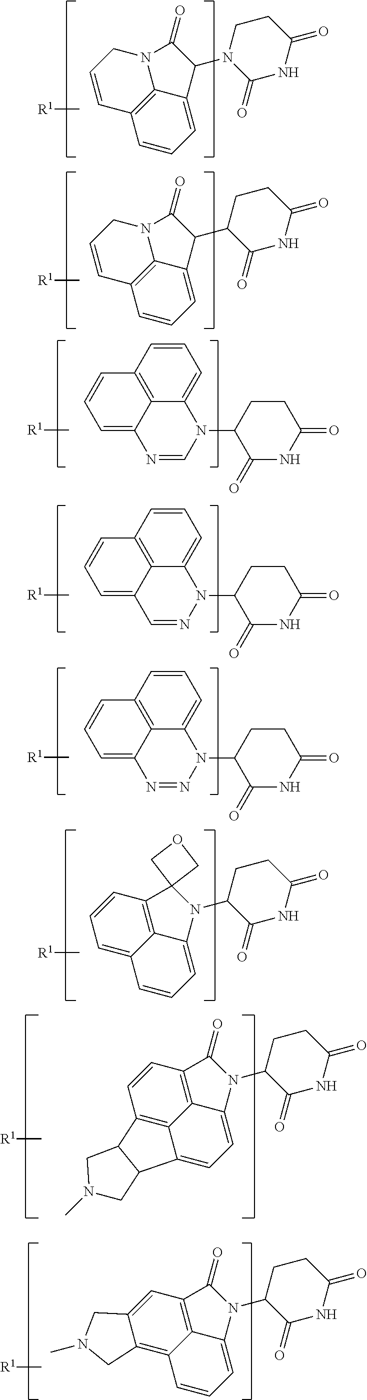

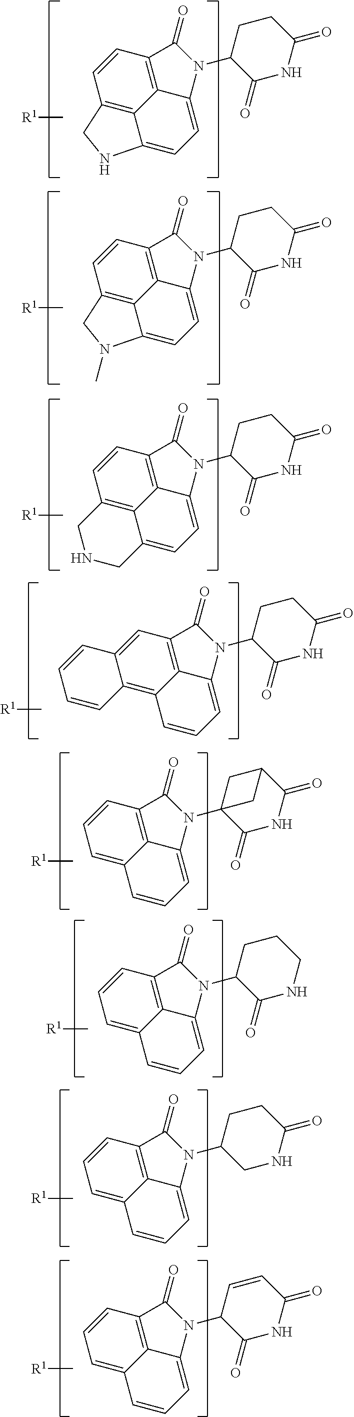

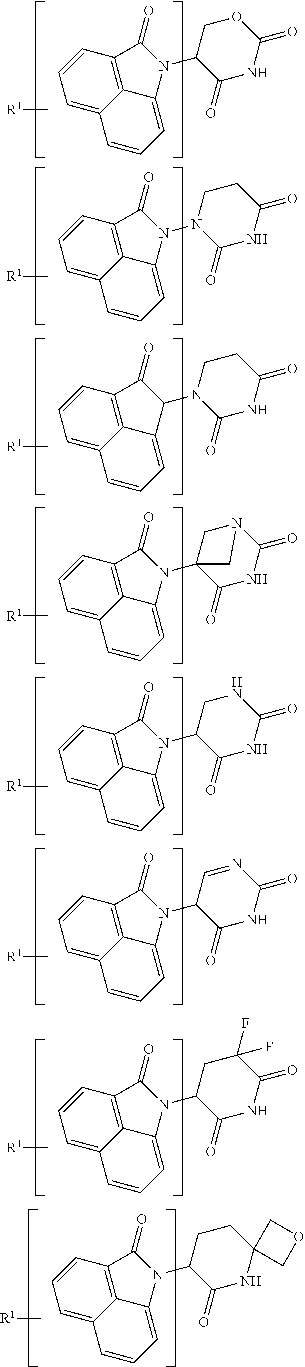

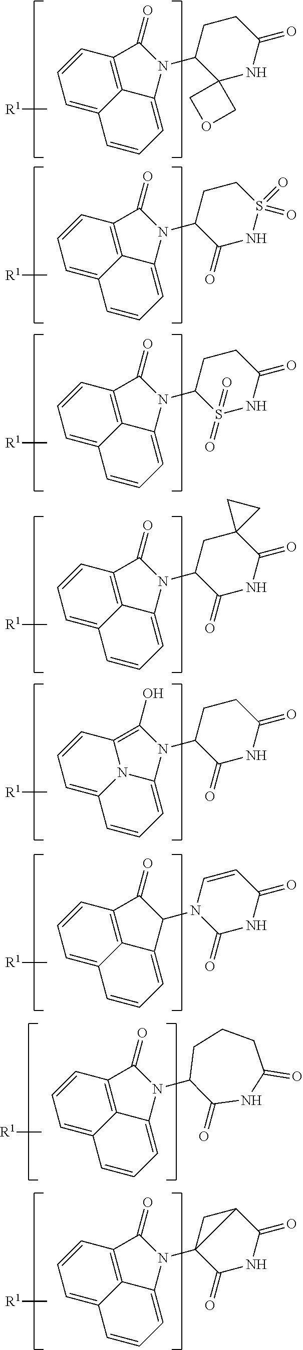

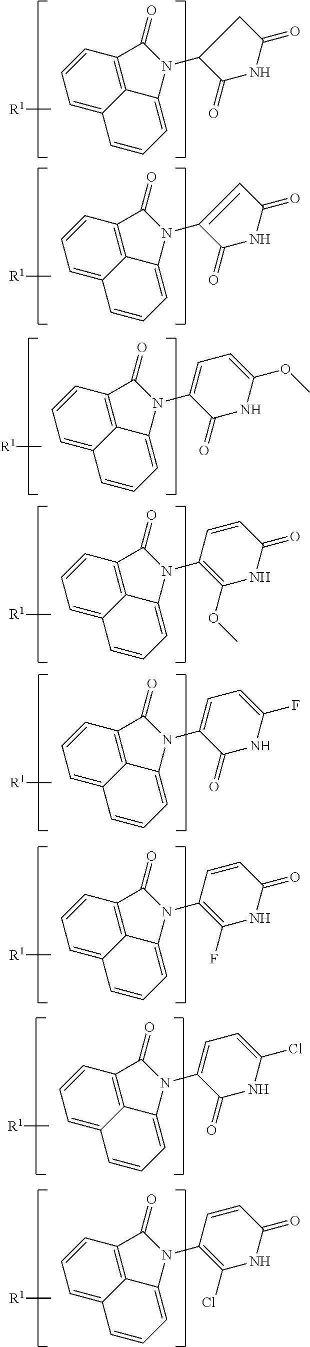

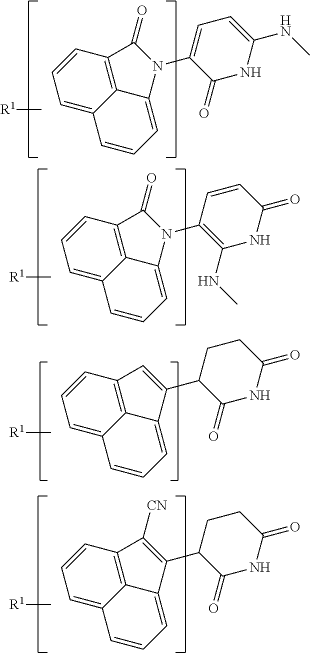

Embodiments of R¹ and R²:

In certain embodiments, R¹ is hydrogen.

In certain embodiments, R¹ is alkyl.

In certain embodiments, R¹ is halogen.

In certain embodiments, R¹ is haloalkyl.

In certain embodiments, R¹ is —OR¹⁰.

In certain embodiments, R¹ is —SR¹⁰.

In certain embodiments, R¹ is —S(O)R¹².

In certain embodiments, R¹ is —SO₂R¹².

In certain embodiments, R¹ is —NR¹⁰R¹¹.

In certain embodiments, R¹ is cyano.

In certain embodiments, R¹ is nitro.

In certain embodiments, R¹ is heteroaryl.

In certain embodiments, R¹ is aryl.

In certain embodiments, R¹ is heterocycle.

In certain embodiments, R² is hydrogen.

In certain embodiments, R² is alkyl.

In certain embodiments, R² is halogen.

In certain embodiments, R² is haloalkyl.

In certain embodiments, R² is —OR¹⁰.

In certain embodiments, R² is —SR¹⁰.

In certain embodiments, R² is —S(O)R¹².

In certain embodiments, R² is —SO₂R¹².

In certain embodiments, R² is —NR¹⁰R¹¹.

In certain embodiments, R² is cyano.

In certain embodiments, R² is nitro.

In certain embodiments, R² is heteroaryl.

In certain embodiments, R² is aryl.

In certain embodiments, R² is heterocycle.

Non-limiting embodiments of R¹′:

In certain embodiments, R^{1′} is alkyl.

In certain embodiments, R^{1′} is halogen

In certain embodiments, R^{1′} is haloalkyl.

In certain embodiments, R^{1′} is —OR¹⁰.

In certain embodiments, R^{1′} is —SR¹⁰.

In certain embodiments, R^{1′} is —S(O)R¹².

In certain embodiments, R^{1′} is —SO₂R¹².

In certain embodiments, R^{1′} is —NR¹⁰R¹¹.

In certain embodiments, R^{1′} is cyano.

In certain embodiments, R^{1′} is nitro

In certain embodiments, R^{1′} is heteroaryl.

In certain embodiments, R^{1′} is aryl.

In certain embodiments, R^{1′} is cycloalkyl.

In certain embodiments, R¹′ is heterocycle.

Non-limiting embodiments of R^{3a}:

In certain embodiments, R^{3a} is hydrogen

In certain embodiments, R^{3a} is alkyl

In certain embodiments, R^{3a} is fluorine.

In certain embodiments, R^{3a} is bromine.

In certain embodiments, R^{3a} is chlorine.

In certain embodiments, R^{3a} is iodine.

In certain embodiments, R^{3a} is haloalkyl.

In certain embodiments, R^{3a} is fluoroalkyl.

In certain embodiments, R^{3a} is chloroalkyl.

In certain embodiments, R^{3a} is bromoalkyl.

In certain embodiments, R^{3a} is iodoalkyl.

Non-limiting embodiments of R³:

In certain embodiments R³ is selected from hydrogen and halogen.

In certain embodiments R³ is selected from alkyl and haloalkyl.

In certain embodiments R³ is hydrogen.

In certain embodiments R³ is halogen.

In certain embodiments R³ is alkyl.

In certain embodiments R³ is haloalkyl.

In certain embodiments R³ is fluorine.

In certain embodiments R³ is chlorine.

In certain embodiments R³ is bromine.

In certain embodiments R³ is iodine.

In certain embodiments R³ is methyl.

In certain embodiments R³ is ethyl.

In certain embodiments R³ is trifluoromethyl.

In certain embodiments R³ is pentafluoroethyl.

In certain embodiments R³ is difluoromethyl.

In certain embodiments R³ is fluoromethyl.

In certain embodiments R³ is combined with an R⁴ group to form a 1 carbon attachment.

In certain embodiments R³ is combined with an R⁴ group to form a 2 carbon attachment.

In certain embodiments R³ is combined with an R⁴ group to form a 3 carbon attachment.

In certain embodiments R³ is combined with an R⁴ group to form a 4 carbon attachment.

In certain embodiments R³ is combined with an R⁴ group to form a double bond.

Non-limiting embodiments of R⁴:

In certain embodiments R⁴ is selected from hydrogen and halogen.

In certain embodiments R⁴ is selected from alkyl and haloalkyl.

In certain embodiments R⁴ is hydrogen.

In certain embodiments R⁴ is halogen.

In certain embodiments R⁴ is alkyl.

In certain embodiments R⁴ is haloalkyl.

In certain embodiments R³ is fluorine.

In certain embodiments R³ is chlorine.

In certain embodiments R³ is bromine.

In certain embodiments R³ is iodine.

In certain embodiments R⁴ is methyl.

In certain embodiments R⁴ is ethyl.

In certain embodiments R⁴ is trifluoromethyl.

In certain embodiments R⁴ is pentafluoroethyl.

In certain embodiments R⁴ is difluoromethyl.

In certain embodiments R⁴ is fluoromethyl.

In certain embodiments R⁴ is combined with an R³ group to form a 1 carbon attachment.

In certain embodiments R⁴ is combined with an R³ group to form a 2 carbon attachment.

In certain embodiments R⁴ is combined with an R³ group to form a 3 carbon attachment.

In certain embodiments R⁴ is combined with an R³ group to form a 4 carbon attachment.

In certain embodiments R⁴ is combined with an R³ group to form a double bond.

Non-limiting embodiments of R^{4a}:

In certain embodiments, R^{4a} is hydrogen

In certain embodiments, R^{4a} is alkyl

In certain embodiments, R^{4a} is fluorine.

In certain embodiments, R^{4a} is bromine.

In certain embodiments, R^{4a} is chlorine.

In certain embodiments, R^{4a} is iodine.

In certain embodiments, R^{4a} is haloalkyl.

In certain embodiments, R^{4a} is fluoroalkyl.

In certain embodiments, R^{4a} is chloroalkyl.

In certain embodiments, R^{4a} is bromoalkyl.

In certain embodiments, R^{4a} is iodoalkyl.

Non-limiting embodiments of R⁵:

In certain embodiments R⁵ is selected from hydrogen and halogen.

In certain embodiments R⁵ is selected from alkyl and haloalkyl.

In certain embodiments R⁵ is hydrogen.

In certain embodiments R⁵ is halogen.

In certain embodiments R⁵ is alkyl.

In certain embodiments R⁵ is haloalkyl.

In certain embodiments R³ is fluorine.

In certain embodiments R³ is chlorine.

In certain embodiments R³ is bromine.

In certain embodiments R³ is iodine.

In certain embodiments R⁵ is methyl.

In certain embodiments R⁵ is ethyl.

In certain embodiments R⁵ is trifluoromethyl.

In certain embodiments R⁵ is pentafluoroethyl.

In certain embodiments R⁵ is difluoromethyl.

In certain embodiments R⁵ is fluoromethyl.

Non-limiting embodiments of R⁶ and R⁷.

In certain embodiments R⁶ is halogen.

In certain embodiments R⁶ is alkyl.

In certain embodiments R⁶ is haloalkyl.

In certain embodiments R⁶ is fluorine.

In certain embodiments R⁶ is chlorine.

In certain embodiments R⁶ is bromine.

In certain embodiments R⁶ is iodine.

In certain embodiments R⁶ is methyl.

In certain embodiments R⁶ is ethyl.

In certain embodiments R⁶ is trifluoromethyl.

In certain embodiments R⁶ is pentafluoroethyl.

In certain embodiments R⁶ is difluoromethyl.

In certain embodiments R⁶ is fluoromethyl.

In certain embodiments R⁶ is —OR¹⁰.

In certain embodiments R⁶ is —SR¹⁰.

In certain embodiments R⁶ is —S(O)R¹².

In certain embodiments R⁶ is —SO₂R¹².

In certain embodiments R⁶ is —NR¹⁰R¹¹.

In certain embodiments R⁶ is pentafluoroethyl.

In certain embodiments R⁶ is difluoromethyl.

In certain embodiments R⁶ is fluoromethyl.

In certain embodiments, R⁶ forms a 3-membered spirocyle with R⁷.

In certain embodiments, R⁶ forms a 4-membered spirocycle with R⁷.

In certain embodiments, R⁶ forms a 4-membered spirocyle with R³.

In certain embodiments, R⁶ forms a 5-membered spirocycle with R³.

In certain embodiments R⁷ is halogen.

In certain embodiments R⁷ is alkyl.

In certain embodiments R⁷ is haloalkyl.

In certain embodiments R⁷ is fluorine.

In certain embodiments R⁷ is chlorine.

In certain embodiments R⁷ is bromine.

In certain embodiments R⁷ is iodine.

In certain embodiments R⁷ is methyl.

In certain embodiments R⁷ is ethyl.

In certain embodiments R⁷ is trifluoromethyl.

In certain embodiments R⁷ is pentafluoroethyl.

In certain embodiments R⁷ is difluoromethyl.

In certain embodiments R⁷ is fluoromethyl.

In certain embodiments R⁷ is —OR¹⁰.

In certain embodiments R⁷ is —SR¹⁰.

In certain embodiments R⁷ is —S(O)R¹².

In certain embodiments R⁷ is —SO₂R¹².

In certain embodiments R⁷ is —NR¹⁰R¹¹.

In certain embodiments R⁷ is pentafluoroethyl.

In certain embodiments R⁷ is difluoromethyl.

In certain embodiments R⁷ is fluoromethyl.

In certain embodiments, R⁷ forms a 3-membered spirocyle with R⁶.

In certain embodiments, R⁷ forms a 4-membered spirocycle with R⁶.

Non-limiting embodiments of R^{6a} and R^{7a}.

In certain embodiments R^{6a} is halogen.

In certain embodiments R^{6a} is alkyl.

In certain embodiments R^{6a} is haloalkyl.

In certain embodiments R^{6a} is fluorine.

In certain embodiments R^{6a} is chlorine.

In certain embodiments R^{6a} is bromine.

In certain embodiments R^{6a} is iodine.

In certain embodiments R^{6a} is methyl.

In certain embodiments R^{6a} is ethyl.

In certain embodiments R^{6a} is trifluoromethyl.

In certain embodiments R^{6a} is pentafluoroethyl.

In certain embodiments R^{6a} is difluoromethyl.

In certain embodiments R^{6a} is fluoromethyl.

In certain embodiments R^{6a} is —OR¹⁰.

In certain embodiments R^{6a} is —SR¹⁰.

In certain embodiments R^{6a} is —S(O)R¹².

In certain embodiments R^{6a} is —SO₂R¹².

In certain embodiments R^{6a} is —NR¹⁰R¹¹.

In certain embodiments R^{6a} is pentafluoroethyl.

In certain embodiments R^{6a} is difluoromethyl.

In certain embodiments R^{6a} is fluoromethyl.

In certain embodiments, R^{6a} forms a 3-membered spirocyle with R^{7a}.

In certain embodiments, R^{6a} forms a 4-membered spirocycle with R^{7a}.

In certain embodiments, R^{6a} forms a 4-membered spirocyle with R^{7a}.

In certain embodiments, R^{6a} forms a 5-membered spirocycle with R^{7a}.

In certain embodiments R^{7a} is halogen.

In certain embodiments R^{7a} is alkyl.

In certain embodiments R^{7a} is haloalkyl.

In certain embodiments R^{7a} is fluorine.

In certain embodiments R^{7a} is chlorine.

In certain embodiments R^{7a} is bromine.

In certain embodiments R^{7a} is iodine.

In certain embodiments R^{7a} is methyl.

In certain embodiments R^{7a} is ethyl.

In certain embodiments R^{7a} is trifluoromethyl.

In certain embodiments R^{7a} is pentafluoroethyl.

In certain embodiments R^{7a} is difluoromethyl.

In certain embodiments R^{7a} is fluoromethyl.

In certain embodiments R^{7a} is —OR¹⁰.

In certain embodiments R^{7a} is —SR¹⁰.

In certain embodiments R^{7a} is —S(O)R¹².

In certain embodiments R^{7a} is —SO₂R¹².

In certain embodiments R^{7a} is —NR¹⁰R¹¹.

In certain embodiments R^{7a} is pentafluoroethyl.

In certain embodiments R^{7a} is difluoromethyl.

In certain embodiments R^{7a} is fluoromethyl.

In certain embodiments, R^{7a} forms a 3-membered spirocyle with R^{6a}.

In certain embodiments, R^{7a} forms a 4-membered spirocycle with R^{6a}.

In certain embodiments, R^{7a} forms a 5-membered spirocycle with R^{6a}.

Non-limiting embodiments of R¹⁰ and R¹¹.

In certain embodiments, R¹⁰ is hydrogen.

In certain embodiments, R¹⁰ is alkyl.

In certain embodiments, R¹⁰ is heterocycle.

In certain embodiments, R¹⁰ is haloalkyl.

In certain embodiments, R¹⁰ is aryl.

In certain embodiments, R¹⁰ is heteroaryl.

In certain embodiments, R¹⁰ is —C(O)R¹².

In certain embodiments, R¹⁰ is —S(O)R¹².

In certain embodiments, R¹⁰ is —SO₂R¹².

In certain embodiments, R¹¹ is hydrogen.

In certain embodiments, R¹¹ is alkyl.

In certain embodiments, R¹¹ is heterocycle.

In certain embodiments, R¹¹ is haloalkyl.

In certain embodiments, R¹¹ is aryl.

In certain embodiments, R¹¹ is heteroaryl.

In certain embodiments, R¹¹ is —C(O)R¹².

In certain embodiments, R¹¹ is —S(O)R¹².

In certain embodiments, R¹¹ is —SO₂R¹².

Non-limiting embodiments of R²:

In certain embodiments, R¹² is hydrogen.

In certain embodiments, R¹² is alkyl.

In certain embodiments, R¹² is heterocycle.

In certain embodiments, R¹² is haloalkyl.

In certain embodiments, R¹² is aryl.

In certain embodiments, R¹² is heteroaryl

In certain embodiments, R¹² is —NR¹³R¹⁴.

In certain embodiments, R¹² is OR¹³.

Non-limiting embodiments of R¹³ and R¹⁴:

In certain embodiments, R¹³ is hydrogen.

In certain embodiments, R¹³ is alkyl.

In certain embodiments, R¹³ is fluoroalkyl.

In certain embodiments, R¹³ is chloroalkyl

In certain embodiments, R¹³ is bromoalkyl.

In certain embodiments, R¹³ is haloalkyl.

In certain embodiments, R¹³ is hydrogen and R¹⁴ is hydrogen.

In certain embodiments, R¹³ is hydrogen and R¹⁴ is alkyl.

In certain embodiments, R¹³ is hydrogen and R¹⁴ is fluoroalkyl.

In certain embodiments, R¹³ is hydrogen and R¹⁴ is bromoalkyl.

In certain embodiments, R¹³ is hydrogen and R¹⁴ is chloroalkyl.

In certain embodiments, R¹³ is alkyl and R¹⁴ is hydrogen.

In certain embodiments, R¹³ is alkyl and R¹⁴ is alkyl.

In certain embodiments, R¹³ is alkyl and R¹⁴ is fluoroalkyl.

In certain embodiments, R¹³ is alkyl and R¹⁴ is bromoalkyl.

In certain embodiments, R¹³ is alkyl and R¹⁴ is chloroalkyl.

In certain embodiments, R¹³ is haloalkyl and R¹⁴ is haloalkyl.

In certain embodiments, R¹³ is alkyl and R¹⁴ is alkyl.

In certain embodiments, R¹⁴ is hydrogen.

In certain embodiments, R¹⁴ is alkyl.

In certain embodiments, R¹⁴ is haloalkyl.

In certain embodiments, R¹⁴ is fluoroalkyl.

In certain embodiments, R¹⁴ is chloroalkyl.

In certain embodiments, R¹⁴ is bromoalkyl.

In certain embodiments, R¹⁴ is hydrogen and R¹³ is hydrogen.

In certain embodiments, R¹⁴ is hydrogen and R¹³ is alkyl.

In certain embodiments, R¹⁴ is hydrogen and R¹³ is fluoroalkyl.

In certain embodiments, R¹⁴ is hydrogen and R¹³ is bromoalkyl.

In certain embodiments, R¹⁴ is hydrogen and R¹³ is chloroalkyl.

In certain embodiments, R¹⁴ is alkyl and R¹³ is hydrogen.

In certain embodiments, R¹⁴ is alkyl and R¹³ is alkyl.

In certain embodiments, R¹⁴ is alkyl and R¹³ is fluoroalkyl.

In certain embodiments, R¹⁴ is alkyl and R¹³ is bromoalkyl.

In certain embodiments, R¹⁴ is alkyl and R¹³ is chloroalkyl.

In certain embodiments, R¹⁴ is haloalkyl and R¹³ is haloalkyl.

In certain embodiments, R¹⁴ is alkyl and R¹³ is alkyl.

Non-limiting embodiments of X²:

In certain embodiments, X² is bond.

In certain embodiments, X² is heterocycle.

In certain embodiments, X² is heteroaryl.

In certain embodiments, X² is aryl.

In certain embodiments, X² is bicycle.

In certain embodiments, X² is alkyl.

In certain embodiments, X² is aliphatic.

In certain embodiments, X² is heteroaliphatic.

In certain embodiments, X² is NR²⁷—.

In certain embodiments, X² is CR⁴⁰R⁴¹—.

In certain embodiments, X² is —C(O)—.

In certain embodiments, X² is —C(NR²⁷)—.

In certain embodiments, X² is —C(S)—.

In certain embodiments, X² is —S(O)—.

In certain embodiments, X² is —S(O)₂—.

In certain embodiments, X² is —S—.

In certain embodiments, X² is a 5-membered aromatic heterocycle with attachment points in a 1,3 orientation.

In certain embodiments, X² is a 5-membered aromatic heterocycle with attachment points in a 1,2 orientation.

In certain embodiments, X² is a 6-membered aromatic heterocycle with attachment points in a 1,2 orientation.

In certain embodiments, X² is a 6-membered aromatic heterocycle with attachment points in a 1,3 orientation.

In certain embodiments, X² is a 6-membered aromatic heterocycle with attachment points in a 1,4 orientation.

In certain embodiments, X² is a 6-membered aromatic heterocycle with attachment points in a 1,3 orientation.

In certain embodiments, X² is a 5-membered heterocycle with attachment points in a 1,2 orientation

In certain embodiments, X² is a 5-membered heterocycle with attachment points in a 1,3 orientation.

In certain embodiments, X² is a 6-membered heterocycle with attachment points in a 1,2 orientation.

In certain embodiments, X² is a 6-membered heterocycle with attachment points in a 1,3 orientation.

In certain embodiments, X² is a 6-membered heterocycle with attachment points in a 1,4 orientation.

In certain embodiments, X² is a bicyclic heterocycle with one heteroatom

In certain embodiments, X² is a bicyclic heterocycle with two heteroatoms.

In certain embodiments, X² is a bicyclic heterocycle with one heteroatom and one attachment is bound to Nitrogen and one is bound to carbon

In certain embodiments, X² is a bicyclic heterocycle with one heteroatom, and both attachment points are bound to carbon

In certain embodiments, X² is a bicyclic heterocycle with two heteroatoms and both points of attachment are bound to Nitrogen.

In certain embodiments, X² is a bicyclic heterocycle with two heteroatoms.

In certain embodiments, X² is a fused bicyclic alkane.

In certain embodiments, X² is a spiro-bicyclic alkane.

Non-limiting embodiments of X³:

In certain embodiments, X³ is bond.

In certain embodiments, X³ is heterocycle.

In certain embodiments, X³ is heteroaryl.

In certain embodiments, X³ is aryl.

In certain embodiments, X³ is bicycle.

In certain embodiments, X³ is NR²⁷—.

In certain embodiments, X³ is CR⁴⁰R⁴¹—.

In certain embodiments, X³ is —C(O)—.

In certain embodiments, X³ is —C(NR²⁷)—.

In certain embodiments, X³ is —C(S)—.

In certain embodiments, X³ is —S(O)—.

In certain embodiments, X³ is —S(O)₂—.

In certain embodiments, X³ is —S—.

In certain embodiments, X³ is a 5-membered aromatic heterocycle with attachment points in a 1,3 orientation.

In certain embodiments, X³ is a 5-membered aromatic heterocycle with attachment points in a 1,2 orientation.

In certain embodiments, X³ is a 6-membered aromatic heterocycle with attachment points in a 1,2 orientation.

In certain embodiments, X³ is a 6-membered aromatic heterocycle with attachment points in a 1,3 orientation.

In certain embodiments, X³ is a 6-membered aromatic heterocycle with attachment points in a 1,4 orientation.

In certain embodiments, X³ is a 6-membered aromatic heterocycle with attachment points in a 1,3 orientation.

In certain embodiments, X³ is a 5-membered heterocycle with attachment points in a 1,2 orientation

In certain embodiments, X³ is a 5-membered heterocycle with attachment points in a 1,3 orientation.

In certain embodiments, X³ is a 6-membered heterocycle with attachment points in a 1,2 orientation.

In certain embodiments, X³ is a 6-membered heterocycle with attachment points in a 1,3 orientation.

In certain embodiments, X³ is a 6-membered heterocycle with attachment points in a 1,4 orientation.

In certain embodiments, X³ is a bicyclic heterocycle with one heteroatom

In certain embodiments, X³ is a bicyclic heterocycle with two heteroatoms.

In certain embodiments, X³ is a bicyclic heterocycle with one heteroatom and one attachment is bound to Nitrogen and one is bound to carbon

In certain embodiments, X³ is a bicyclic heterocycle with one heteroatom, and both attachment points are bound to carbon

In certain embodiments, X³ is a bicyclic heterocycle with two heteroatoms and both points of attachment are bound to Nitrogen.

In certain embodiments, X³ is a bicyclic heterocycle with two heteroatoms.

In certain embodiments, X³ is a fused bicyclic alkane.

In certain embodiments, X³ is a spiro-bicyclic alkane.

In certain embodiments, X³ is selected from:

Non-limiting embodiments of R¹⁵, R¹⁶, and R¹⁷:

In certain embodiments, R¹⁵ is bond.

In certain embodiments, R¹⁵ is alkyl.

In certain embodiments, R¹⁵ is —C(O)—.

In certain embodiments, R¹⁵ is —C(O)O—.

In certain embodiments, R¹⁵ is —OC(O)—.

In certain embodiments, R¹⁵ is —SO₂—.

In certain embodiments, R¹⁵ is —S(O)—.

In certain embodiments, R¹⁵ is —C(S)—.

In certain embodiments, R¹⁵ is C(O)NR⁷—.

In certain embodiments, R¹⁵ is —NR²⁷C(O)—.

In certain embodiments, R¹⁵ is —O—.

In certain embodiments, R¹⁵ is —S—.

In certain embodiments, R¹⁵ is —NR²⁷—.

In certain embodiments, R¹⁵ is C(R⁴⁰R⁴¹)—.

In certain embodiments, R¹⁵ is P(O)(OR²⁶)O—.

In certain embodiments, R¹⁵ is —P(O)(OR²⁶)—.

In certain embodiments, R¹⁵ is bicycle.

In certain embodiments, R¹⁵ is alkene.

In certain embodiments, R¹⁵ is alkyne.

In certain embodiments, R¹⁵ is haloalkyl.

In certain embodiments, R¹⁵ is alkoxy.

In certain embodiments, R¹⁵ is aryl

In certain embodiments, R¹⁵ is heterocycle.

In certain embodiments, R¹⁵ is heteroaliphatic.

In certain embodiments, R¹⁵ is heteroaryl.

In certain embodiments, R¹⁵ is lactic acid

In certain embodiments, R¹⁵ is glycolic acid.

In certain embodiments, R¹⁵ is arylalkyl.

In certain embodiments, R¹⁵ is heterocyclealkyl.

In certain embodiments, R¹⁵ is heteroarylalkyl.

In certain embodiments, R¹⁶ is bond.

In certain embodiments, R¹⁶ is alkyl.

In certain embodiments, R¹⁶ is —C(O)—.

In certain embodiments, R¹⁶ is —C(O)O—.

In certain embodiments, R¹⁶ is —OC(O)—.

In certain embodiments, R¹⁶ is —SO₂—.

In certain embodiments, R¹⁶ is —S(O)—.

In certain embodiments, R¹⁶ is —C(S)—.

In certain embodiments, R¹⁶ is C(O)NR²⁷—.

In certain embodiments, R¹⁶ is —NR²⁷C(O)—.

In certain embodiments, R¹⁶ is —O—.

In certain embodiments, R¹⁶ is —S—.

In certain embodiments, R¹⁶ is —N²⁷—.

In certain embodiments, R¹⁶ is C(R⁴⁰R⁴¹)—.

In certain embodiments, R¹⁶ is P(O)(OR²⁶)O—.

In certain embodiments, R¹⁶ is —P(O)(OR²⁶)—.

In certain embodiments, R¹⁶ is bicycle.

In certain embodiments, R¹⁶ is alkene.

In certain embodiments, R¹⁶ is alkyne.

In certain embodiments, R¹⁶ is haloalkyl.

In certain embodiments, R¹⁶ is alkoxy.

In certain embodiments, R¹⁶ is aryl

In certain embodiments, R¹⁶ is heterocycle.

In certain embodiments, R¹⁶ is heteroaliphatic.

In certain embodiments, R¹⁶ is heteroaryl.

In certain embodiments, R¹⁶ is lactic acid

In certain embodiments, R¹⁶ is glycolic acid.

In certain embodiments, R¹⁶ is arylalkyl.

In certain embodiments, R¹⁶ is heterocyclealkyl.

In certain embodiments, R¹⁶ is heteroarylalkyl.

In certain embodiments, R¹⁷ is bond.

In certain embodiments, R¹⁷ is alkyl.

In certain embodiments, R¹⁷ is —C(O)—.

In certain embodiments, R¹⁷ is —C(O)O—.

In certain embodiments, R¹⁷ is —OC(O)—.

In certain embodiments, R¹⁷ is —SO₂—.

In certain embodiments, R¹⁷ is —S(O)—.

In certain embodiments, R¹⁷ is —C(S)—.

In certain embodiments, R¹⁷ is C(O)NR²⁷—.

In certain embodiments, R¹⁷ is —NR²⁷C(O)—.

In certain embodiments, R¹⁷ is —O—.

In certain embodiments, R¹⁷ is —S—.

In certain embodiments, R¹⁷ is —NR²⁷—.

In certain embodiments, R¹⁷ is C(R⁴⁰R⁴¹)—.

In certain embodiments, R¹⁷ is P(O)(OR²⁶)O—.

In certain embodiments, R¹⁷ is —P(O)(OR²⁶)—.

In certain embodiments, R¹⁷ is bicycle.

In certain embodiments, R¹⁷ is alkene.

In certain embodiments, R¹⁷ is alkyne.

In certain embodiments, R¹⁷ is haloalkyl.

In certain embodiments, R¹⁷ is alkoxy.

In certain embodiments, R¹⁷ is aryl

In certain embodiments, R¹⁷ is heterocycle.

In certain embodiments, R¹⁷ is heteroaliphatic.

In certain embodiments, R¹⁷ is heteroaryl.

In certain embodiments, R¹⁷ is lactic acid

In certain embodiments, R¹⁷ is glycolic acid.

In certain embodiments, R¹⁷ is arylalkyl.

In certain embodiments, R¹⁷ is heterocyclealkyl.

In certain embodiments, R¹⁷ is heteroarylalkyl.

Non-limiting embodiments of R⁸:

In certain embodiments R¹⁸ is hydrogen.

In certain embodiments R¹⁸ is alkyl.

In certain embodiments R¹⁸ is alkene.

In certain embodiments R¹⁸ is alkyne.

In certain embodiments R¹⁸ is hydroxy.

In certain embodiments R¹⁸ is azide.

In certain embodiments R¹⁸ is amino.

In certain embodiments R¹⁸ is halogen.

In certain embodiments R¹⁸ is haloalkyl

In certain embodiments R¹⁸ is —OR¹⁰.

In certain embodiments R¹⁹ is —SR¹⁰.

In certain embodiments R¹⁸ is —S(O)R¹².

In certain embodiments R¹⁸ is —SO₂R¹².

In certain embodiments R¹⁸ is —NR¹⁰R¹¹.

In certain embodiments R¹⁸ is cyano.

In certain embodiments R¹⁸ is nitro.

In certain embodiments R¹⁸ is heteroaryl.

In certain embodiments R¹⁸ is aryl.

In certain embodiments R¹⁸ is arylalkyl.

In certain embodiments R¹⁸ is cycloalkyl.

In certain embodiments R¹⁸ is heterocycle.

In certain embodiments R¹⁸ is bond.

In certain embodiments R¹⁸ is bond.

In certain embodiments R¹⁸ is bond.

In certain embodiments R¹⁸ is bond.

In certain embodiments R¹⁸ is bond.

In certain embodiments R¹⁸ is bond.

Non-limiting embodiments of R²⁰, R²¹, R²², R²³, and R²⁴:

In a certain embodiment, R²⁰ is bond.

In a certain embodiment, R²⁰ is alkyl.

In a certain embodiment, R²⁰ is —C(O)—.

In a certain embodiment, R²⁰ is —C(O)O—.

In a certain embodiment, R²⁰ is —OC(O)—.

In a certain embodiment, R²⁰ is —SO₂—.

In a certain embodiment, R²⁰ is —S(O)—.

In a certain embodiment, R²⁰ is —C(S)—.

In a certain embodiment, R²⁰ is —C(O)NR²⁷—.

In a certain embodiment, R²⁰ is —NR²⁷C(O)—.

In a certain embodiment, R²⁰ is —O—.

In a certain embodiment, R²⁰ is —S—.

In a certain embodiment, R²⁰ is —NR²⁷—.

In a certain embodiment, R²⁰ is oxyalkylene.

In a certain embodiment, R²⁰ is —C(R⁴⁰R⁴⁰)—.

In a certain embodiment, R²⁰ is —P(O)(OR²⁶)O—.

In a certain embodiment, R²⁰ is —P(O)(OR²⁶)—.

In a certain embodiment, R²⁰ is bicycle.

In a certain embodiment, R²⁰ is alkene.

In a certain embodiment, R²⁰ is alkyne.

In a certain embodiment, R²⁰ is haloalkyl.

In a certain embodiment, R²⁰ is alkoxy.

In a certain embodiment, R²⁰ is aryl.

In a certain embodiment, R²⁰ is heterocycle.

In a certain embodiment, R²⁰ is aliphatic

In a certain embodiment, R²⁰ is heteroaliphatic.

In a certain embodiment, R²⁰ is heteroaryl.

In a certain embodiment, R²⁰ is lactic acid.

In a certain embodiment, R²⁰ is glycolic acid.

In a certain embodiment, R²⁰ is carbocycle.

In a certain embodiment, R²¹ is bond.

In a certain embodiment, R²¹ is alkyl.

In a certain embodiment, R²¹ is —C(O)—.

In a certain embodiment, R²¹ is —C(O)O—.

In a certain embodiment, R²¹ is —OC(O)—.

In a certain embodiment, R²¹ is —SO₂—.

In a certain embodiment, R²¹ is —S(O)—.

In a certain embodiment, R²¹ is —C(S)—.

In a certain embodiment, R²¹ is —C(O)NR²⁷—.

In a certain embodiment, R²¹ is —NR²⁷C(O)—.

In a certain embodiment, R²¹ is —O—.

In a certain embodiment, R²¹ is —S—.

In a certain embodiment, R²¹ is —NR²⁷—.

In a certain embodiment, R²¹ is oxyalkylene.

In a certain embodiment, R²¹ is —C(R⁴⁰R⁴⁰)—.

In a certain embodiment, R²¹ is —P(O)(OR²⁶)O—.

In a certain embodiment, R²¹ is —P(O)(OR²⁶)—.

In a certain embodiment, R²¹ is bicycle.

In a certain embodiment, R²¹ is alkene.

In a certain embodiment, R²¹ is alkyne.

In a certain embodiment, R²¹ is haloalkyl.

In a certain embodiment, R²¹ is alkoxy.

In a certain embodiment, R²¹ is aryl.

In a certain embodiment, R²¹ is heterocycle.

In a certain embodiment, R²¹ is aliphatic

In a certain embodiment, R²¹ is heteroaliphatic.

In a certain embodiment, R²¹ is heteroaryl.

In a certain embodiment, R²¹ is lactic acid.

In a certain embodiment, R²¹ is glycolic acid.

In a certain embodiment, R²¹ is carbocycle.

In a certain embodiment, R²² is bond.

In a certain embodiment, R²² is alkyl.

In a certain embodiment, R²² is —C(O)—.

In a certain embodiment, R²² is —C(O)O—.

In a certain embodiment, R²² is —OC(O)—.

In a certain embodiment, R²² is —SO₂—.

In a certain embodiment, R²² is —S(O)—.

In a certain embodiment, R²² is —C(S)—.

In a certain embodiment, R²² is —C(O)NR²⁷—.

In a certain embodiment, R²² is —NR²⁷C(O)—.

In a certain embodiment, R²² is —O—.

In a certain embodiment, R²² is —S—.

In a certain embodiment, R²² is —NR²⁷—.

In a certain embodiment, R²² is oxyalkylene.

In a certain embodiment, R²² is —C(R⁴⁰R⁴⁰)—.

In a certain embodiment, R²² is —P(O)(OR²⁶)O—.

In a certain embodiment, R²² is —P(O)(OR²⁶)—.

In a certain embodiment, R²² is bicycle.

In a certain embodiment, R²² is alkene.

In a certain embodiment, R²² is alkyne.

In a certain embodiment, R²² is haloalkyl.

In a certain embodiment, R²² is alkoxy.

In a certain embodiment, R²² is aryl.

In a certain embodiment, R²² is heterocycle.

In a certain embodiment, R²² is aliphatic

In a certain embodiment, R²² is heteroaliphatic.

In a certain embodiment, R²² is heteroaryl.

In a certain embodiment, R²² is lactic acid.

In a certain embodiment, R²² is glycolic acid.

In a certain embodiment, R²² is carbocycle.

In a certain embodiment, R²³ is bond.

In a certain embodiment, R²³ is alkyl.

In a certain embodiment, R²³ is —C(O)—.

In a certain embodiment, R²³ is —C(O)O—.

In a certain embodiment, R²³ is —OC(O)—.

In a certain embodiment, R²³ is —SO₂—.

In a certain embodiment, R²³ is —S(O)—.

In a certain embodiment, R²³ is —C(S)—.

In a certain embodiment, R²³ is —C(O)NR²⁷—.

In a certain embodiment, R²³ is —NR²⁷C(O)—.

In a certain embodiment, R²³ is —O—.

In a certain embodiment, R²³ is —S—.

In a certain embodiment, R²³ is —NR²⁷—.

In a certain embodiment, R²³ is oxyalkylene.

In a certain embodiment, R²³ is —C(R⁴⁰R⁴⁰)—.

In a certain embodiment, R²³ is —P(O)(OR²⁶)O—.

In a certain embodiment, R²³ is —P(O)(OR²⁶)—.

In a certain embodiment, R²³ is bicycle.

In a certain embodiment, R²³ is alkene.

In a certain embodiment, R²³ is alkyne.

In a certain embodiment, R²³ is haloalkyl.

In a certain embodiment, R²³ is alkoxy.

In a certain embodiment, R²³ is aryl.

In a certain embodiment, R²³ is heterocycle.

In a certain embodiment, R²³ is aliphatic

In a certain embodiment, R²³ is heteroaliphatic.

In a certain embodiment, R²³ is heteroaryl.

In a certain embodiment, R²³ is lactic acid.

In a certain embodiment, R²³ is glycolic acid.

In a certain embodiment, R²³ is carbocycle.

In a certain embodiment, R²⁴ is bond.

In a certain embodiment, R²⁴ is alkyl.

In a certain embodiment, R²⁴ is —C(O)—.

In a certain embodiment, R²⁴ is —C(O)O—.

In a certain embodiment, R²⁴ is —OC(O)—.

In a certain embodiment, R²⁴ is —SO₂—.

In a certain embodiment, R²⁴ is —S(O)—.

In a certain embodiment, R²⁴ is —C(S)—.

In a certain embodiment, R²⁴ is —C(O)NR²⁷—.

In a certain embodiment, R²⁴ is —NR²⁷C(O)—.

In a certain embodiment, R²⁴ is —O—.

In a certain embodiment, R²⁴ is —S—.

In a certain embodiment, R²⁴ is —NR²⁷—.

In a certain embodiment, R²⁴ is oxyalkylene.

In a certain embodiment, R²⁴ is —C(R⁴⁰R⁴⁰)—.

In a certain embodiment, R²⁴ is —P(O)(OR²⁶)O—.

In a certain embodiment, R²⁴ is —P(O)(OR²⁶)—.

In a certain embodiment, R²⁴ is bicycle.

In a certain embodiment, R²⁴ is alkene.

In a certain embodiment, R²⁴ is alkyne.

In a certain embodiment, R²⁴ is haloalkyl.

In a certain embodiment, R²⁴ is alkoxy.

In a certain embodiment, R²⁴ is aryl.

In a certain embodiment, R²⁴ is heterocycle.

In a certain embodiment, R²⁴ is aliphatic

In a certain embodiment, R²⁴ is heteroaliphatic.

In a certain embodiment, R²⁴ is heteroaryl.

In a certain embodiment, R²⁴ is lactic acid.

In a certain embodiment, R²⁴ is glycolic acid.

In a certain embodiment, R²⁴ is carbocycle.

Non-limiting embodiments of R²⁵:

In a certain embodiment, R²⁵ is aliphatic.

In a certain embodiment, R²⁵ is aryl.

In a certain embodiment, R²⁵ is heteroaryl.

In a certain embodiment, R²⁵ is hydrogen.

Non-limiting embodiments of R²⁶:

In certain embodiments, R²⁶ is hydrogen.

In certain embodiments, R²⁶ is alkyl.

In certain embodiments, R²⁶ is arylalkyl.

In certain embodiments, R²⁶ is heteroarylalkyl.

In certain embodiments, R²⁶ is alkene

In certain embodiments, R²⁶ is alkyne

In certain embodiments, R²⁶ is aryl

In certain embodiments, R²⁶ is heteroaryl.

In certain embodiments, R²⁶ is heterocycle.

In certain embodiments, R²⁶ is aliphatic.

In certain embodiments, R²⁶ is heteroaliphatic.

Non-limiting embodiments of R²⁷

In certain embodiments, R²⁷ is hydrogen.

In certain embodiments, R²⁷ is alkyl.

In certain embodiments, R²⁷ is aliphatic.

In certain embodiments, R²⁷ is heteroaliphatic.

In certain embodiments, R²⁷ is heterocycle.

In certain embodiments, R²⁷ is aryl.

In certain embodiments, R²⁷ is heteroaryl.

In certain embodiments, R²⁷ is —C(O)(aliphatic)

In certain embodiments, R²⁷ is —C(O)(aryl)

In certain embodiments, R²⁷ is —C(O)(heteroaliphatic)

In certain embodiments, R²⁷ is —C(O)(heteroaryl)

In certain embodiments, R²⁷ is alkene.

In certain embodiments, R²⁷ is alkyne.

Non-limiting embodiments of R²⁸.

In certain embodiments, R²⁸ is alkyl.

In certain embodiments, R²⁸ is alkene.

In certain embodiments, R²⁸ is alkyne.

In certain embodiments, R²⁸ is hydroxy.

In certain embodiments, R²⁸ is azide

In certain embodiments, R²⁸ is amino.

In certain embodiments, R²⁸ is halogen.

In certain embodiments, R²⁸ is haloalkyl.

In certain embodiments, R²⁸ is —OR¹⁰.

In certain embodiments, R²⁸ is —SR¹⁰.

In certain embodiments, R²⁸ is —S(O)R¹².

In certain embodiments, R²⁸ is —SO₂R¹².

In certain embodiments, R²⁸ is —NR¹⁰R¹¹.

In certain embodiments, R²⁸ is cyano.

In certain embodiments, R²⁸ is nitro.

In certain embodiments, R²⁸ is heteroaryl.

In certain embodiments, R²⁸ is aryl

In certain embodiments, R²⁸ is arylalkyl

In certain embodiments, R²⁸ is cycloalkyl

In certain embodiments, R²⁸ is heterocycle.

Non-limiting embodiments of R⁴⁰

In certain embodiments, R⁴⁰ is hydrogen.

In certain embodiments, R⁴⁰ is R²⁷.

In certain embodiments, R⁴⁰ is alkyl.

In certain embodiments, R⁴⁰ is alkene.

In certain embodiments, R⁴⁰ is alkyne.

In certain embodiments, R⁴⁰ is fluorine.

In certain embodiments, R⁴⁰ is bromine.

In certain embodiments, R⁴⁰ is chlorine.

In certain embodiments, R⁴⁰ is hydroxyl.

In certain embodiments, R⁴⁰ is azide.

In certain embodiments, R⁴⁰ is amino.

In certain embodiments, R⁴⁰ is cyano

In certain embodiments, R⁴⁰ is alkoxy

In certain embodiments, R⁴⁰ is-NH(alkyl).

In certain embodiments, R⁴⁰ is-NH(aliphatic).

In certain embodiments, R⁴⁰ is —N(aliphatic)₂.

In certain embodiments, R⁴⁰ is-N(alkyl)₂.

In certain embodiments, R⁴⁰ is —NHSO₂(alkyl).

In certain embodiments, R⁴⁰ is —NHSO₂(aliphatic).

In certain embodiments, R⁴⁰ is —N(alkyl)SO₂alkyl.

In certain embodiments, R⁴⁰ is —N(aliphatic)SO₂alkyl.

In certain embodiments, R⁴⁰ is —NHSO₂(aryl).

In certain embodiments, R⁴⁰ is —NHSO₂(heteroaryl).

In certain embodiments, R⁴⁰ is —NHSO₂(heterocycle).

In certain embodiments, R⁴⁰ is —N(alkyl)SO₂(aryl).

In certain embodiments, R⁴⁰ is —N(alkyl)SO₂(heteroaryl).

In certain embodiments, R⁴⁰ is —N(alkyl)SO₂(heterocycle).

In certain embodiments, R⁴⁰ is—NHSO₂alkenyl.

In certain embodiments, R⁴⁰ is —N(alkyl)SO₂alkenyl.

In certain embodiments, R⁴⁰ is—NHSO₂alkynyl.

In certain embodiments, R⁴⁰ is —N(alkyl)SO₂alkynyl.

In certain embodiments, R⁴⁰ is haloalkyl.

In certain embodiments, R⁴⁰ is aliphatic

In certain embodiments, R⁴⁰ is heteroaliphatic.

In certain embodiments, R⁴⁰ is aryl.

In certain embodiments, R⁴⁰ is heteroaryl.

In certain embodiments, R⁴⁰ is heterocycle.

In certain embodiments, R⁴⁰ is cycloalkyl.

Non-limiting embodiments of R⁴¹:

In a certain embodiment, R⁴¹ is aliphatic.

In a certain embodiment, R⁴¹ is aryl.

In a certain embodiment, R⁴¹ is heteroaryl.

In a certain embodiment, R⁴¹ is hydrogen.

### Additional Embodiments of the Present Invention

1. In certain embodiments a compound selected from the following Formulas is provided:

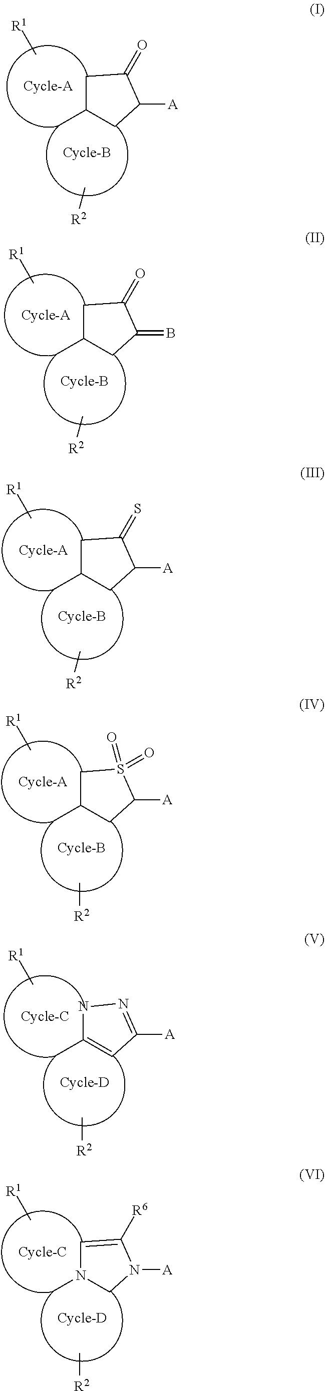

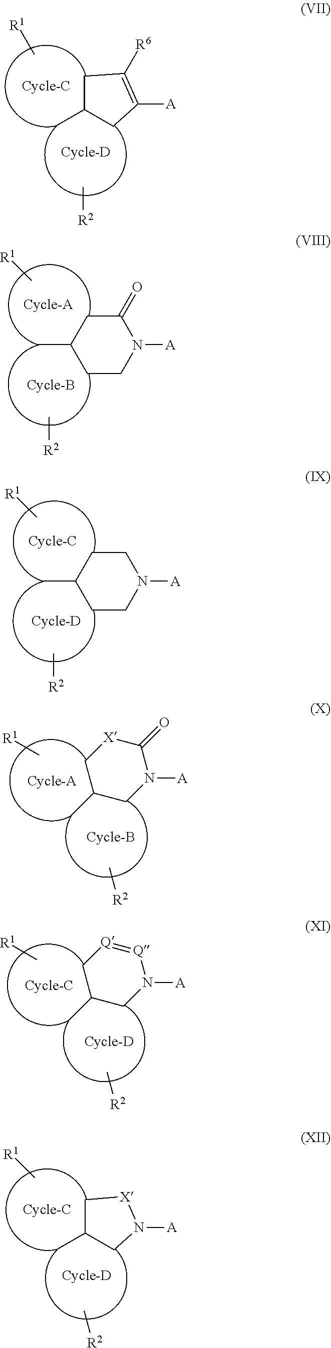

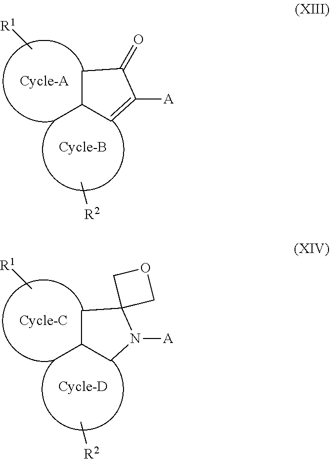

or a pharmaceutically acceptable salt, N-oxide, isotopic derivative, or prodrug thereof, optionally in a pharmaceutically acceptable carrier to form a composition;
wherein:
A is selected from

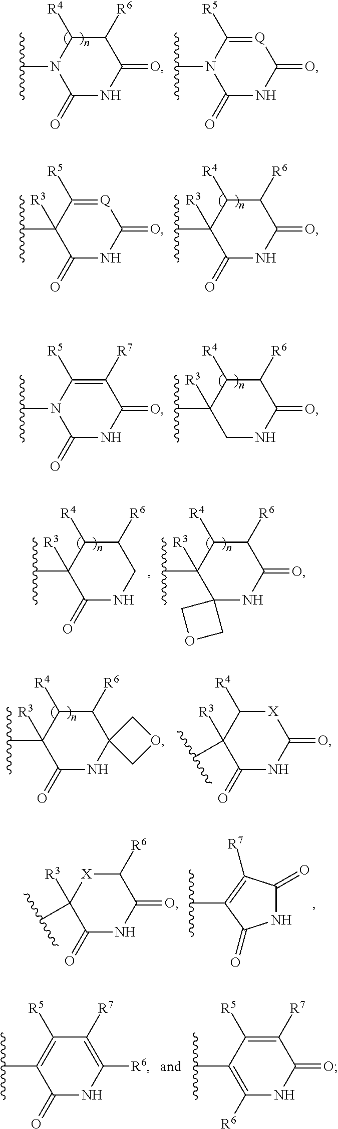

B is selected from

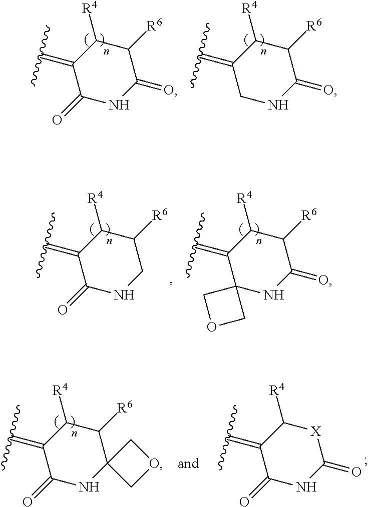

n is 0, 1, or 2;
X is NR¹⁰, NR^{6′} O, or S;
X′ is NR¹⁰, O, CH₂, or S;
Q is CR⁷ or N;
Q′ and Q″ are each independently selected from CR⁶ and N;
Cycle-A is a fused ring selected from phenyl, 5- or 6-membered heteroaryl, 5- to 8-membered heterocycle, 5- to 8-membered cycloalkyl, and 5- to 8-membered cycloalkenyl wherein Cycle-A is optionally substituted with 1, 2, or 3 substituents independently selected from R¹ as allowed by valence;
Cycle-B is a fused ring selected from phenyl, 5- or 6-membered heteroaryl, 5- to 8-membered heterocycle, 5- to 8-membered cycloalkyl, and 5- to 8-membered cycloalkenyl wherein Cycle-B is optionally substituted with 1, 2, or 3 substituents independently selected from R² as allowed by valence;
In certain embodiments Cycle-A is a fused ring selected from phenyl, 5- or 6-membered heteroaryl, 5- to 6-membered heterocycle, 5- to 6-membered cycloalkyl, or 5- to 6-membered cycloalkenyl, wherein Cycle-A is optionally substituted with 1, 2, or 3 substituents independently selected from R¹ as allowed by valence;
In certain embodiments Cycle-B is a fused ring selected from phenyl, 5- or 6-membered heteroaryl, 5- to 6-membered heterocycle, 5- to 6-membered cycloalkyl, or 5- to 6-membered cycloalkenyl, wherein Cycle-B is optionally substituted with 1, 2, or 3 substituents independently selected from R² as allowed by valence;
Cycle-C is a fused ring selected from phenyl, 5- or 6-membered heteroaryl, 5- to 6-membered heterocycle, 5- to 6-membered cycloalkyl, and 5- to 6-membered cycloalkenyl wherein Cycle-C is optionally substituted with 1, 2, or 3 substituents independently selected from R¹ as allowed by valence;
Cycle-D is a fused ring selected from phenyl, 5- or 6-membered heteroaryl, 5 to 6-membered heterocycle, 5- to 6-membered cycloalkyl, and 5- to 6-membered cycloalkenyl wherein Cycle-D is optionally substituted with 1, 2, or 3 substituents independently selected from R² as allowed by valence;
R¹ and R² are each independently at each instance selected from
(a) hydrogen, alkyl, halogen, haloalkyl, —OR¹⁰, —SR¹⁰, —S(O)R¹², —SO₂R¹², —NR¹⁰R¹¹, cyano, nitro, heteroaryl, aryl, cycloalkyl, and heterocycle wherein each heteroaryl, aryl, cycloalkyl, and heterocycle is optionally substituted with 1, 2, 3, or 4 substituents independently selected from R⁴⁰;
(b)

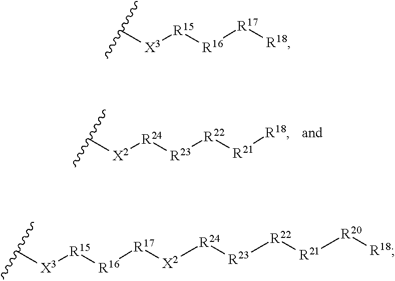

(c) if allowed by valence and stability, a divalent moiety such as O, S, or ═NR²⁵; and
wherein an R¹ group may optionally be combined with another R¹ group or an R² group to form a fused cycle or bicycle which may bridge Cycle-A and Cycle-B or Cycle-C and Cycle-D, as appropriate and desired;
R³ is hydrogen, alkyl, halogen, or haloalkyl;
or R³ and R⁶ are combined to form a 1 or 2 carbon attachment, for example, when R³ and R⁶ form a 1 carbon attachment

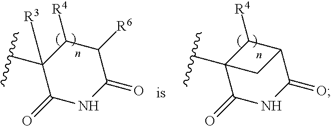

or R³ and R⁴ are combined to form a 1, 2, 3, or 4 carbon attachment, for example when R³ and R⁴ form a 1 carbon attachment

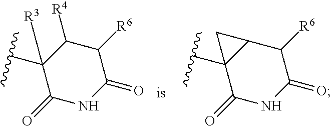

or R³ and an R⁴ group adjacent to R³ are combined to form a double bond;
each R⁴ is independently selected from hydrogen, alkyl, halogen, and haloalkyl;
R⁵ is hydrogen, alkyl, halogen, or haloalkyl;
R⁶ and R⁷ are independently selected at each instance from hydrogen, alkyl, halogen, haloalkyl, —OR¹⁰, —SR¹⁰, —S(O)R¹², —SO₂R¹², and —NR¹⁰R¹¹; wherein if R⁶ and R⁷ are on the same carbon atom they can optionally form a 3- to 4-membered spirocycle ring.
R^{6′} is hydrogen, alkyl, or haloalkyl;
or R³ and R^{6′} are combined to form a 1 or 2 carbon attachment;
each R¹⁰ and R¹¹ are independently selected from hydrogen, alkyl, haloalkyl, heterocycle, aryl, heteroaryl, —C(O)R¹², —S(O)R¹², and —SO₂R¹²;
each R¹² is independently selected from hydrogen, alkyl, haloalkyl, heterocycle, aryl, heteroaryl, —NR¹³R¹⁴, and OR¹³;
R¹³ and R¹⁴ are each independently selected from hydrogen, alkyl, and haloalkyl;
each X² is a bivalent moiety selected from bond, heterocycle, aryl, heteroaryl, bicycle, alkyl, aliphatic, heteroaliphatic, —NR²⁷—, —CR⁴⁰R⁴¹—, —O—, —C(O)—, —C(NR²⁷)—, —C(S)—, —S(O)—, —S(O)₂—, and —S—; each of which heterocycle, aryl, heteroaryl, and bicycle is optionally substituted with 1, 2, 3, or 4 substituents independently selected from R⁴⁰;
X³ is a bivalent moiety selected from bond, heterocycle, aryl, heteroaryl, bicycle, —NR²⁷—, —CR⁴⁰R⁴¹—, —O—, —C(O)—, —C(NR²⁷)—, —C(S)—, —S(O)—, —S(O)₂—, —S—, arylalkyl, heterocyclealkyl, or heteroarylalkyl (in either direction); each of which heterocycle, aryl, heteroaryl, and bicycle may be substituted with 1, 2, 3, or 4 substituents independently selected from R⁴⁰;
R¹⁵, R¹⁶, and R¹⁷ are independently at each occurrence selected from the group consisting of a bond, alkyl (which in certain embodiments is a carbocycle), —C(O)—, —C(O)O—, —OC(O)—, —SO₂—, —S(O)—, —C(S)—, —C(O)NR²⁷—, —NR²⁷C(O)—, —O—, —S—, —NR²⁷—, —C(R⁴⁰R⁴¹)—, —P(O)(OR²⁶)O—, —P(O)(OR²⁶)—, bicycle, alkene, alkyne, haloalkyl, alkoxy, aryl, heterocycle, aliphatic, heteroaliphatic, heteroaryl, lactic acid, glycolic acid arylalkyl, heterocyclealkyl and heteroarylalkyl (in either direction); each of which is optionally substituted with 1, 2, 3, or 4 substituents independently selected from R⁴⁰;
R¹⁸ is selected from hydrogen, alkyl, alkene, alkyne, hydroxy, azide, amino, halogen, haloalkyl, —OR¹⁰, —SR¹⁰, —S(O)R¹², —SO₂R¹², —NR¹⁰R¹¹, cyano, nitro, heteroaryl, aryl, arylalkyl, cycloalkyl, and heterocycle wherein each heteroaryl, aryl, arylalkyl, cycloalkyl, and heterocycle is optionally substituted with 1, 2, 3, or 4 substituents independently selected from R⁴⁰;
R²⁰, R²¹, R²², R²³, and R²⁴ are independently at each occurrence selected from the group consisting of a bond, alkyl, —C(O)—, —C(O)O—, —OC(O)—, —SO₂—, —S(O)—, —C(S)—, —C(O)NR²⁷—, —NR²⁷C(O)—, —O—, —S—, —NR²⁷—, oxyalkylene, —C(R⁴⁰R⁴⁰)—, —P(O)(OR²⁶)O—, —P(O)(OR²⁶)—, bicycle, alkene, alkyne, haloalkyl, alkoxy, aryl, heterocycle, aliphatic, heteroaliphatic, heteroaryl, lactic acid, glycolic acid, and carbocycle; each of which is optionally substituted with 1, 2, 3, or 4 substituents independently selected from R⁴⁰;
R²⁵ is aliphatic (including alkyl), aryl, heteroaryl, or hydrogen;
R²⁶ is independently at each occurrence selected from the group consisting of hydrogen, alkyl, arylalkyl, heteroarylalkyl, alkene, alkyne, aryl, heteroaryl, heterocycle, aliphatic and heteroaliphatic;
R²⁷ is independently at each occurrence selected from the group consisting of hydrogen, alkyl, aliphatic, heteroaliphatic, heterocycle, aryl, heteroaryl, —C(O)(aliphatic, aryl, heteroaliphatic or heteroaryl), —C(O)O(aliphatic, aryl, heteroaliphatic, or heteroaryl), alkene, and alkyne;
R⁴⁰ is independently at each occurrence selected from the group consisting of hydrogen, R²⁷, alkyl, alkene, alkyne, fluoro, bromo, chloro, hydroxyl, alkoxy, azide, amino, cyano, —NH(aliphatic, including alkyl), —N(aliphatic, including alkyl)₂, —NHSO₂(aliphatic, including alkyl), —N(aliphatic, including alkyl)SO₂alkyl, —NHSO₂(aryl, heteroaryl or heterocycle), —N(alkyl)SO₂(aryl, heteroaryl or heterocycle),—NHSO₂alkenyl, —N(alkyl)SO₂alkenyl, —NHSO₂alkynyl, —N(alkyl)SO₂alkynyl, haloalkyl, aliphatic, heteroaliphatic, aryl, heteroaryl, heterocycle, and cycloalkyl; and
R⁴¹ is aliphatic (including alkyl), aryl, heteroaryl, or hydrogen.

2. In certain embodiments a compound selected from the following Formulas is provided:

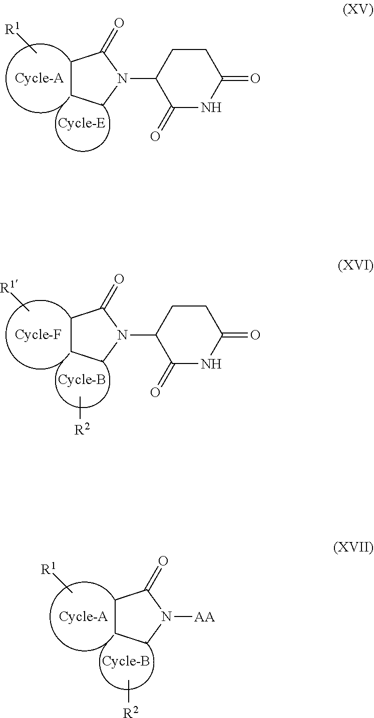

or a pharmaceutically acceptable salt, N-oxide, isotopic derivative, or prodrug thereof, optionally in a pharmaceutically acceptable carrier to form a composition;
wherein
AA is selected from

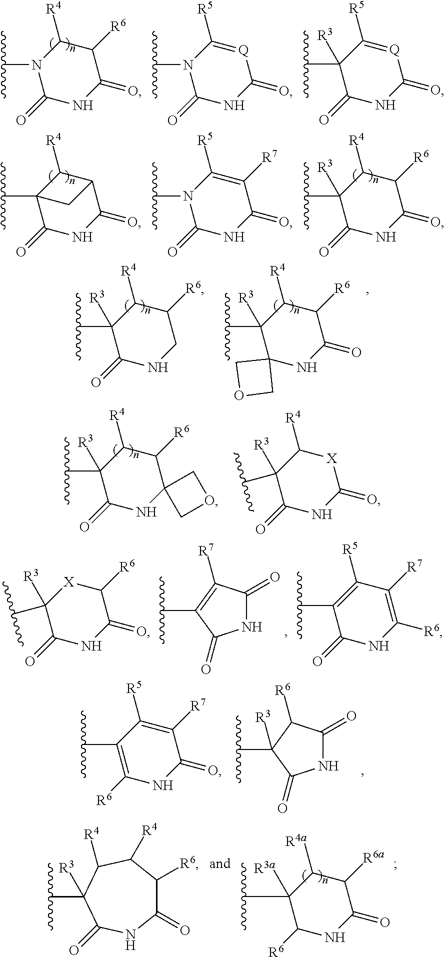

Cycle-E is selected from
(a)

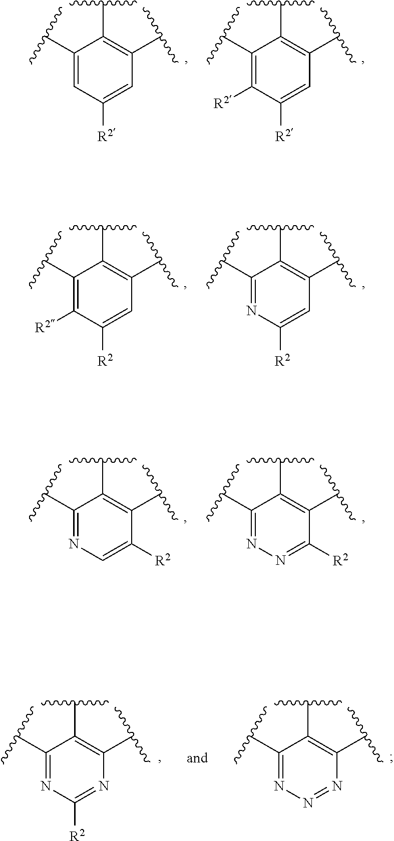

and
(b) a fused ring selected from 5-membered heteroaryl, 5- to 8-membered heterocycle, 5- to 8-membered cycloalkyl, or 5- to 8-membered cycloalkenyl optionally substituted with 1, 2, or 3 substituents independently selected from R² as allowed by valence;
Cycle-F is selected from
(a) phenyl substituted with 1, 2, or 3 substituents independently selected from R¹′; and
(b) a fused ring selected from 5- or 6-membered heteroaryl, 5- to 8-membered heterocycle, 5- to 8-membered cycloalkyl, or 5- to 8-membered cycloalkenyl optionally substituted with 1, 2, or 3 substituents independently selected from R¹ as allowed by valence;
R¹′ is independently at each instance selected from
(a) alkyl, halogen, haloalkyl, —OR¹⁰, —SR¹⁰, —S(O)R¹², —SO₂R¹², —NR¹⁰R¹¹, cyano, nitro, heteroaryl, aryl, cycloalkyl, and heterocycle wherein each heteroaryl, aryl and heterocycle is optionally substituted with 1, 2, 3, or 4 substituents independently selected from R⁴⁰;
(b)

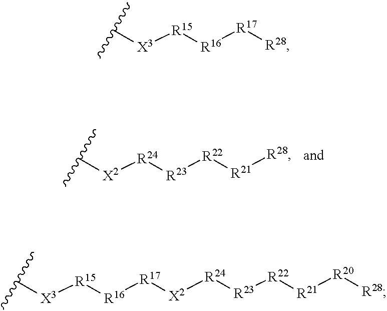

and
(c) if allowed by valence and stability, a divalent moiety such as O, S, or ═NR¹⁵; and
wherein an R¹′ group may optionally be combined with another R¹′ group or an R² group to form a fused cycle or bicycle which may bridge Cycle-A and Cycle-E;
R²′ is independently at each instance selected from alkyl, halogen, haloalkyl, —OR¹⁰, —SR¹⁰, —S(O)R¹², —SO₂R¹², —NR¹⁰R¹¹, cyano, nitro, heteroaryl, aryl, and heterocycle; or alternatively, if allowed by valence and stability, R²′ may be a divalent moiety such as O, S, or ═NR⁵; and wherein an R² group may optionally be combined with another R²′ group or an R¹ group to form a fused cycle or bicycle which may bridge Cycle-A and Cycle-E;
R²″ is independently at each instance selected from heteroaryl, aryl, and heterocycle, and wherein each heteroaryl, aryl and heterocycle is optionally substituted with 1, 2, 3, or 4 substituents independently selected from R⁴⁰ and wherein an R²″ group may optionally be combined with an R¹ group or an R² group to form a fused cycle or bicycle which may bridge Cycle-A and Cycle-E;
R^{3a} is hydrogen, alkyl, halogen, or haloalkyl;
or R^{3a} and R^{6a} are combined to form a 1 or 2 carbon attachment, for example, when R^{3a} and R^{6a} form a 1 carbon attachment

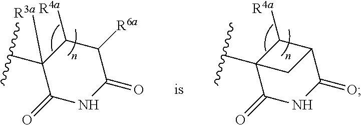

or R^{3a} and R^{4a} are combined to form a 1, 2, 3, or 4 carbon attachment, for example when
R^{3a} and R^{4a} form a 1 carbon attachment

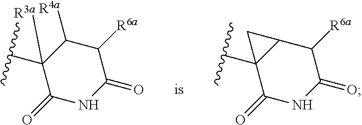

or R^{3a} and an R^{4a} group adjacent to R^{3a} are combined to form a double bond;
R^{4a} is selected from hydrogen, alkyl, halogen, and haloalkyl;
R^{6s} is selected from hydrogen, alkyl, halogen, haloalkyl, —OR¹⁰, —SR¹⁰, —S(O)R¹², —SO₂R¹², and —NR¹⁰R¹¹;
wherein at least one of R^{3a}, R^{4a}, and R^{6a} is not hydrogen;
R²⁸ is selected from alkyl, alkene, alkyne, hydroxy, azide, amino, halogen, haloalkyl, —OR¹⁰, —SR¹⁰, —S(O)R¹², —SO₂R¹², —NR¹⁰R¹¹, cyano, nitro, heteroaryl, aryl, arylalkyl, cycloalkyl, and heterocycle wherein each heteroaryl, aryl, arylalkyl, cycloalkyl, and heterocycle is optionally substituted with 1, 2, 3, or 4 substituents independently selected from R⁴⁰;
wherein if at least one of R¹⁵, R¹⁶, R¹⁷, and R²⁰ is not bond, then R²⁸ can be hydrogen; and
wherein all other variables are as defined herein.

3. The compound of embodiment 2, wherein Cycle-F is phenyl substituted with 1, 2, or 3 substituents independently selected from R¹′.

4. The compound of embodiment 3, wherein R¹′ is selected from alkyl, halogen, and haloalkyl.

5. The compound of embodiment 3, wherein R¹′ is selected from —OR¹⁰, —SR¹⁰, —S(O)R¹², —SO₂R¹², —NR¹⁰R¹¹.

6. The compound of embodiment 3, wherein R¹′ is selected from alkyl, halogen, and haloalkyl.

7. The compound of embodiment 3, wherein R¹′ is selected from heteroaryl, aryl, and heterocycle.

8. The compound of embodiment 3, wherein two R¹′ substituents are combined to form a fused phenyl ring.

9. The compound of embodiment 3, wherein at least one R¹′ is alkyl.

10. The compound of embodiment 3, wherein at least one R¹′ is halogen.

11. The compound of embodiment 3, wherein one R^{1′} is

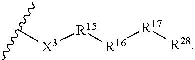

12. The compound of embodiment 3, wherein one R^{1′} is

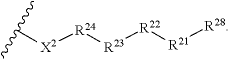

13. The compound of embodiment 3, wherein one R^{1′} is

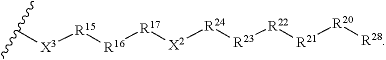

14. The compound of embodiment 2, wherein Cycle E is selected from:

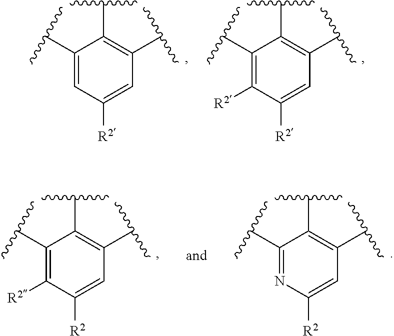

15. The compound of embodiment 14, wherein R² is selected from alkyl, halogen, and haloalkyl.

16. The compound of embodiment 14, wherein R²′ is selected from —OR¹⁰, —SR¹⁰, —S(O)R¹², —SO₂R¹², —NR¹⁰R¹¹.

17. The compound of embodiment 14, wherein R²′ is selected from alkyl, halogen, and haloalkyl.

18. The compound of embodiment 14, wherein R²′ is selected from heteroaryl, aryl, and heterocycle.

19. The compound of embodiment 14, wherein two R²′ substituents are combined to form a fused phenyl ring.

20. The compound of embodiment 14, wherein at least one R²′ is alkyl.

21. The compound of embodiment 14, wherein at least one R²′ is halogen.

22. The compound of any one of embodiments 11-21, wherein at least one of X³ and X² are bond.

23. The compound of any one of embodiments 11-21, wherein at least one of X³ and X² are —O—.

24. The compound of any one of embodiments 11-21, wherein at least one of X³ and X² are —S—.

25. The compound of any one of embodiments 11-21, wherein at least one of X³ and X² are —NR²⁷—.

26. The compound of any one of embodiments 11-25, wherein at least one of R¹⁵ and R²⁴ is bond.

27. The compound of any one of embodiments 11-26, wherein at least one of R¹⁶ and R²³ is bond.

28. The compound of any one of embodiments 11-27, wherein at least one of R¹⁷ and R²² is bond.

29. The compound of any one of embodiments 11-25, wherein no more than 4 substituents selected from R¹⁵, R¹⁶, R¹⁷, R¹⁹, R²⁰, R²¹, R²², R²³, and R²⁴ are selected to be bond.

30. The compound of any one of embodiments 11-25, wherein no more than 3 substituents selected from R¹⁵, R¹⁶, R¹⁷, R¹⁹, R²⁰, R²¹, R²², R²³, and R²⁴ are selected to be bond.

31. The compound of any one of embodiments 11-25, wherein no more than 2 substituents selected from R¹⁵, R¹⁶, R¹⁷, R¹⁹, R²⁰, R²¹, R²², R²³, and R²⁴ are selected to be bond.

32. The compound of any one of embodiments 11-25, wherein no more than 1 substituent selected from R¹⁵, R¹⁶, R¹⁷, R¹⁹, R²⁰, R²¹, R²², R²³, and R²⁴ is selected to be bond.

33. The compound of any one of embodiments 1-32, wherein Cycle-A is a fused ring selected from phenyl, 5- or 6-membered heteroaryl, 5- to 6-membered heterocycle, 5- to 6-membered cycloalkyl, or 5- to 6-membered cycloalkenyl, wherein Cycle-A is optionally substituted with 1, 2, or 3 substituents independently selected from R¹ as allowed by valence.

34. The compound of any one of embodiments 1-32, wherein Cycle-A is a fused ring selected from phenyl or 6-membered heteroaryl, wherein Cycle-A is optionally substituted with 1, 2, or 3 substituents independently selected from R¹ as allowed by valence.

35. The compound of any one of embodiments 1-32, wherein Cycle-A is phenyl optionally substituted with 1, 2, or 3 substituents independently selected from R¹ as allowed by valence.

36. The compound of any one of embodiments 1-32, wherein Cycle-A is 6-membered heteroaryl optionally substituted with 1, 2, or 3 substituents independently selected from R¹ as allowed by valence.

37. The compound of any one of embodiments 1-36, wherein Cycle-B is a fused ring selected from phenyl, 5- or 6-membered heteroaryl, 5- to 6-membered heterocycle, 5- to 6-membered cycloalkyl, or 5- to 6-membered cycloalkenyl, wherein Cycle-B is optionally substituted with 1, 2, or 3 substituents independently selected from R¹ as allowed by valence.

38. The compound of any one of embodiments 1-36, wherein Cycle-B is a fused ring selected from phenyl or 6-membered heteroaryl, wherein Cycle-B is optionally substituted with 1, 2, or 3 substituents independently selected from R¹ as allowed by valence.

39. The compound of any one of embodiments 1-36, wherein Cycle-B is phenyl optionally substituted with 1, 2, or 3 substituents independently selected from R¹ as allowed by valence.

40. The compound of any one of embodiments 1-36, wherein Cycle-B is 6-membered heteroaryl optionally substituted with 1, 2, or 3 substituents independently selected from R¹ as allowed by valence.

41. The compound of any one of embodiments 1-40, wherein R⁵ is hydrogen.

42. The compound of any one of embodiments 1-40, wherein R⁵ is alkyl.

43. The compound of any one of embodiments 1-40, wherein R⁵ is halogen.

44. The compound of any one of embodiments 1-40, wherein R⁵ is haloalkyl.

45. The compound of any one of embodiments 1-44, wherein R⁷ is hydrogen.

46. The compound of any one of embodiments 1-44, wherein R⁷ is halogen, haloalkyl, or alkyl.

47. The compound of any one of embodiments 1-44, wherein R⁷ is —OR¹⁰, —SR¹⁰, or —NR¹⁰R¹¹.

48. The compound of any one of embodiments 1-44, wherein R⁷ is —S(O)R¹² or —SO₂R¹².

49. The compound of any one of embodiments 1-48, wherein there is 4 R² substituents.

50. The compound of any one of embodiments 1-48, wherein there is 3 R² substituents.

51. The compound of any one of embodiments 1-48, wherein there is 2 R² substituents.

52. The compound of any one of embodiments 1-48, wherein there is 1 R² substituent.

53. The compound of any one of embodiments 1-52, wherein R² is selected from alkyl, halogen, and haloalkyl.

54. The compound of any one of embodiments 1-52, wherein R² is selected from —OR¹⁰, —SR¹⁰, —S(O)R¹², —SO₂R¹², —NR¹⁰R¹¹.

55. The compound of any one of embodiments 1-52, wherein R² is selected from alkyl, halogen, and haloalkyl.

56. The compound of any one of embodiments 1-52, wherein R² is selected from heteroaryl, aryl, and heterocycle.

57. The compound of any one of embodiments 1-51, wherein two R² substituents are combined to form a fused phenyl ring.

58. The compound of any one of embodiments 1-52, wherein at least one R² is alkyl.

59. The compound of any one of embodiments 1-52, wherein at least one R² is halogen.

60. The compound of any one of embodiments 1-52, wherein one R² is

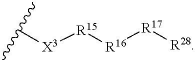

61. The compound of any one of embodiments 1-52, wherein one R² is

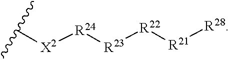

62. The compound of any one of embodiments 1-52, wherein one R² is

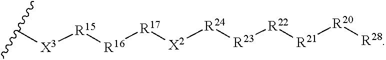

63. The compound of any one of embodiments 1-62, wherein R³ is hydrogen.

64. The compound of any one of embodiments 1-62, wherein R³ is alkyl.

65. The compound of any one of embodiments 1-62, wherein R³ is haloalkyl.

66. The compound of any one of embodiments 1-62, wherein R³ and R⁶ are combined to form a one carbon attachment.

67. The compound of any one of embodiments 1-62, wherein R³ and R⁶ are combined to form a two carbon attachment.

68. The compound of any one of embodiments 1-65, wherein R⁶ is hydrogen.

69. The compound of any one of embodiments 1-65, wherein R⁶ is alkyl.

70. The compound of any one of embodiments 1-65, wherein R⁶ is haloalkyl.

71. The compound of any one of embodiments 1-70, wherein at least one R⁴ is hydrogen.

72. The compound of any one of embodiments 1-70, wherein at least one R⁴ is alkyl.

73. The compound of any one of embodiments 1-70, wherein at least one R⁴ is haloalkyl.

74. The compound of any one of embodiments 1-70, wherein n is 0.

75. The compound of any one of embodiments 1-73, wherein n is 1.

76. The compound of any one of embodiments 1-73, wherein n is 2.

77. The compound of any one of embodiments 1-76, wherein there is 4 R¹ substituents.

78. The compound of any one of embodiments 1-76, wherein there is 3 R¹ substituents.

79. The compound of any one of embodiments 1-76, wherein there is 2 R¹ substituents.

80. The compound of any one of embodiments 1-76, wherein there is 1 R¹ substituent.

81. The compound of any one of embodiments 1-80, wherein R¹ is selected from alkyl, halogen, and haloalkyl.

82. The compound of any one of embodiments 1-80, wherein R¹ is selected from —OR¹⁰, —SR¹⁰, —S(O)R¹², —SO₂R¹², —NR¹⁰R¹¹.

83. The compound of any one of embodiments 1-80, wherein R¹ is selected from alkyl, halogen, and haloalkyl.

84. The compound of any one of embodiments 1-80, wherein R¹ is selected from heteroaryl, aryl, and heterocycle.

85. The compound of any one of embodiments 1-79, wherein two R² substituents are combined to form a fused phenyl ring.

86. The compound of any one of embodiments 1-80, wherein at least one R² is alkyl.

87. The compound of any one of embodiments 1-80, wherein at least one R² is halogen.

88. The compound of any one of embodiments 1-80, wherein one R² is

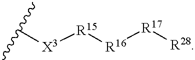

89. The compound of any one of embodiments 1-80, wherein one R² is

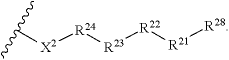

90. The compound of any one of embodiments 1-80, wherein one R² is

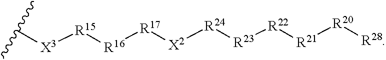

91. The compound of any one of embodiments 1 and 3-90, wherein the compound is of Formula:

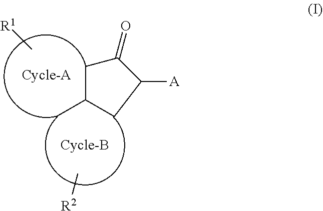

or a pharmaceutically acceptable salt, N-oxide, isotopic derivative, or prodrug thereof, optionally in a pharmaceutically acceptable carrier to form a composition;

92. The compound of any one of embodiments 1 and 3-90, wherein the compound is of Formula:

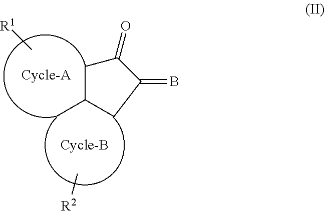

or a pharmaceutically acceptable salt, N-oxide, isotopic derivative, or prodrug thereof, optionally in a pharmaceutically acceptable carrier to form a composition;

93. The compound of any one of embodiments 1 and 3-90, wherein the compound is of

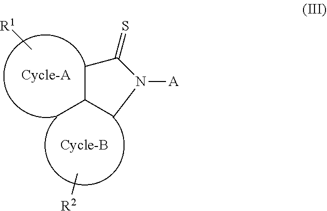

or a pharmaceutically acceptable salt, N-oxide, isotopic derivative, or prodrug thereof, optionally in a pharmaceutically acceptable carrier to form a composition.

94. The compound of any one of embodiments 1 and 3-90, wherein the compound is of Formula:

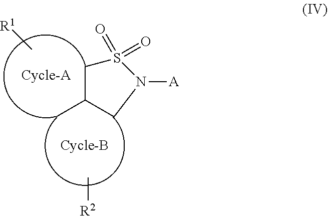

or a pharmaceutically acceptable salt, N-oxide, isotopic derivative, or prodrug thereof, optionally in a pharmaceutically acceptable carrier to form a composition.

95. The compound of any one of embodiments 1 and 3-90, wherein the compound is of Formula:

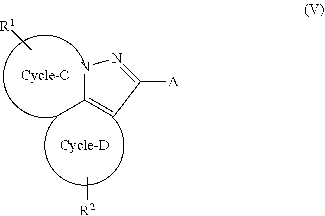

or a pharmaceutically acceptable salt, N-oxide, isotopic derivative, or prodrug thereof, optionally in a pharmaceutically acceptable carrier to form a composition.

96. The compound of any one of embodiments 1 and 3-90, wherein the compound is of

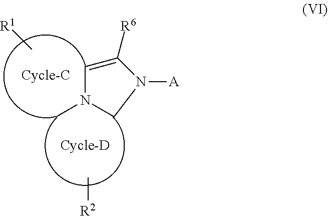

or a pharmaceutically acceptable salt, N-oxide, isotopic derivative, or prodrug thereof, optionally in a pharmaceutically acceptable carrier to form a composition.

97. The compound of any one of embodiments 1 and 3-90, wherein the compound is of Formula:

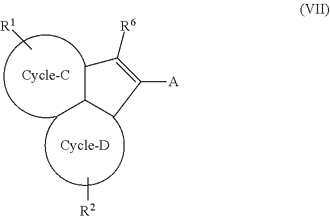

or a pharmaceutically acceptable salt, N-oxide, isotopic derivative, or prodrug thereof, optionally in a pharmaceutically acceptable carrier to form a composition.

98. The compound of any one of embodiments 1 and 3-90, wherein the compound is of Formula:

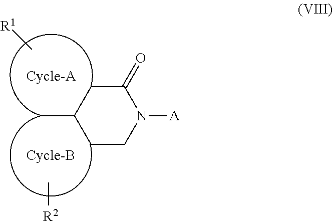

or a pharmaceutically acceptable salt, N-oxide, isotopic derivative, or prodrug thereof, optionally in a pharmaceutically acceptable carrier to form a composition.

99. The compound of any one of embodiments 1 and 3-90, wherein the compound is of

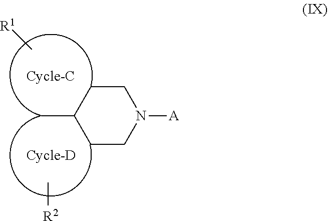

or a pharmaceutically acceptable salt, N-oxide, isotopic derivative, or prodrug thereof, optionally in a pharmaceutically acceptable carrier to form a composition.

100. The compound of any one of embodiments 1 and 3-90, wherein the compound is of Formula:

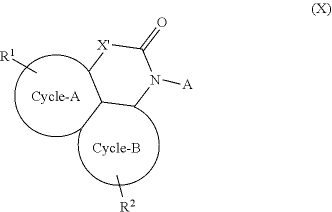

or a pharmaceutically acceptable salt, N-oxide, isotopic derivative, or prodrug thereof, optionally in a pharmaceutically acceptable carrier to form a composition.

101. The compound of any one of embodiments 1 and 3-90, wherein the compound is of Formula:

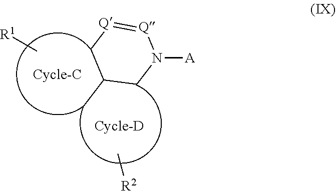

or a pharmaceutically acceptable salt, N-oxide, isotopic derivative, or prodrug thereof, optionally in a pharmaceutically acceptable carrier to form a composition.

102. The compound of any one of embodiments 1 and 3-90, wherein the compound is of Formula:

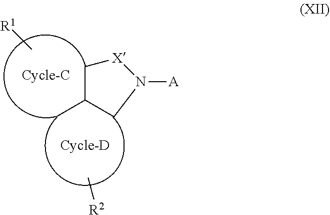

or a pharmaceutically acceptable salt, N-oxide, isotopic derivative, or prodrug thereof, optionally in a pharmaceutically acceptable carrier to form a composition.

103. The compound of any one of embodiments 1 and 3-90, wherein the compound is of Formula:

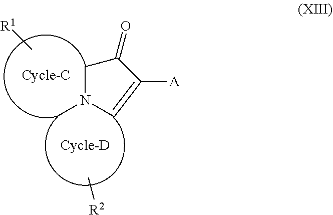

or a pharmaceutically acceptable salt, N-oxide, isotopic derivative, or prodrug thereof, optionally in a pharmaceutically acceptable carrier to form a composition.

104. The compound of any one of embodiments 1 and 3-90, wherein the compound is of Formula:

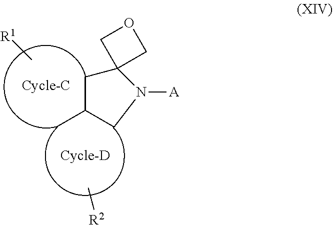

or a pharmaceutically acceptable salt, N-oxide, isotopic derivative, or prodrug thereof, optionally in a pharmaceutically acceptable carrier to form a composition.

105. The compound of any one of embodiments 2-90, wherein the compound is of Formula:

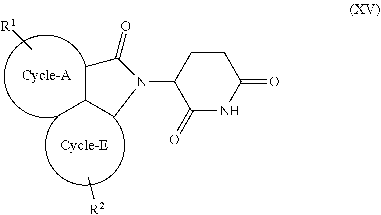

or a pharmaceutically acceptable salt, N-oxide, isotopic derivative, or prodrug thereof, optionally in a pharmaceutically acceptable carrier to form a composition.

106. The compound of any one of embodiments 2-90, wherein the compound is of Formula:

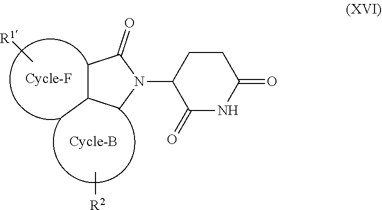

or a pharmaceutically acceptable salt, N-oxide, isotopic derivative, or prodrug thereof, optionally in a pharmaceutically acceptable carrier to form a composition.

107. The compound of any one of embodiments 2-90, wherein the compound is of Formula:

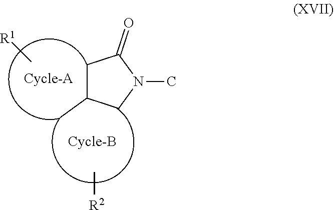

or a pharmaceutically acceptable salt, N-oxide, isotopic derivative, or prodrug thereof, optionally in a pharmaceutically acceptable carrier to form a composition.

108. In certain embodiments a pharmaceutical composition is provided comprising a compound of any one of embodiments 1-107 and a pharmaceutically acceptable excipient.

109. In certain embodiments a method of treating a medical disorder in a patient is provided comprising administering an effective amount of a compound of any one of embodiments 1-107 or a pharmaceutically acceptable salt thereof or a pharmaceutical composition of embodiment 108 to the patient.

110. The method of embodiment 109, wherein the disorder is abnormal cellular proliferation.

111. The method of embodiment 109, wherein the disorder is a neurogenerative disease.

112. The method of embodiment 109, wherein the disorder is an autoimmune diseases.

113. The method of any one of embodiments 109-112 wherein the patient is a human.

### Non-Limiting Embodiments of R¹, R¹′, R², and R²′

In certain embodiments each R¹, R¹′, R², and R²′ are independently selected from alkyl, halogen, haloalkyl, —OR¹⁰, —SR¹⁰, —S(O)R¹², —SO₂R¹², —NR¹⁰R¹¹, cyano, and nitro.

In certain embodiments each R¹ and R² are independently selected from hydrogen, alkyl, halogen, and haloalkyl.

In certain embodiments each R¹, R¹′, R², and R²′ are independently selected from halogen, —OR¹⁰, —SR¹⁰, —S(O)R¹², —SO₂R¹², —NR¹⁰R¹¹, cyano, and nitro.

In certain embodiments each R¹, R¹′, R², and R²′ are independently selected from halogen, —S(O)R¹², —SO₂R¹², cyano, and nitro.

In certain embodiments each R¹, R¹′, R², and R²′ are independently selected from alkyl, haloalkyl, —OR¹⁰, and —SR¹⁰.

In certain embodiments each R¹, R¹′, R², and R²′ are independently selected from alkyl, haloalkyl, and cyano.

In certain embodiments each R¹ and R² is hydrogen.

In certain embodiments R¹ is hydrogen.

In certain embodiments R² is hydrogen.

In certain embodiments one of R¹, R¹′, R², and R²′ is alkyl.

In certain embodiments one of R¹, R¹′, R², and R²′ is halogen.

In certain embodiments one of R¹, R¹′, R², and R²′ is haloalkyl.

In certain embodiments one of R¹, R¹′, R², and R²′ is —OR¹⁰.

In certain embodiments one of R¹, R¹′, R², and R²′ is —SR¹⁰.

In certain embodiments one of R¹, R¹′, R², and R² ′ is —S(O)R¹².

In certain embodiments one of R¹, R¹′, R², and R²′ is —SO₂R¹².

In certain embodiments one of R¹, R¹′, R², and R²′ is —NR¹⁰R¹¹.

In certain embodiments one of R¹, R¹′, R², and R²′ is cyano.

In certain embodiments one of R¹, R¹′, R², and R²′ is nitro.

In certain embodiments one of R¹, R¹′, R², and R²′ is heteroaryl.

In certain embodiments one of R¹, R¹′, R², and R²′ is aryl.

In certain embodiments one of R¹, R¹′, R², and R²′ is heterocyclic.

In certain embodiments each R¹, R¹′, R², and R²′ is alkyl.

In certain embodiments each R¹, R¹′, R², and R²′ is halogen.

In certain embodiments each R¹, R¹′, R², and R²′ is haloalkyl.

In certain embodiments each R¹, R¹′, R², and R²′ is —OR¹⁰.

In certain embodiments each R¹, R¹′, R², and R²′ is —SR¹⁰.

In certain embodiments each R¹, R¹′, R², and R²′ is —S(O)R¹².

In certain embodiments each R¹, R¹′, R², and R²′ is —SO₂R¹².

In certain embodiments each R¹, R¹′, R², and R²′ is —NR¹⁰R¹¹.

In certain embodiments each R¹, R¹′, R², and R²′ is cyano.

In certain embodiments each R¹, R¹′, R², and R²′ is nitro.

In certain embodiments each R¹, R¹′, R², and R²′ is heteroaryl.

In certain embodiments each R¹, R¹′, R², and R²′ is aryl.

In certain embodiments each R¹, R¹′, R², and R²′ is heterocyclic.

In certain embodiments there is only one R¹ substituent on Cycle-A or Cycle-C.

In certain embodiments there are only two R¹ substituents on Cycle-A or Cycle-C.

In certain embodiments there are three R¹ substituents on Cycle-A or Cycle-C.

In certain embodiments, there is only one R¹ substituent on Cycle-A and the R¹ substituent is hydrogen.

In certain embodiments there is only one R¹′ substituent on Cycle-F.

In certain embodiments there are only two R¹′ substituents on Cycle-F.

In certain embodiments there are three R¹′ substituents on Cycle-F.

In certain embodiments there is only one R² substituent on Cycle-B.

In certain embodiments there are only two R² substituents on Cycle-B.

In certain embodiments there are three R² substituents on Cycle-B.

In certain embodiments there is only one R² substituent on Cycle-D.

In certain embodiments there are only two R² substituents on Cycle-D.

In certain embodiments there are three R² substituents on Cycle-D.

In certain embodiments there is only one R² substituent on Cycle-E.

In certain embodiments there are only two R² substituents on Cycle-E.

In certain embodiments there are three R² substituents on Cycle-E.

In certain embodiments there is only one R²′ substituent on Cycle-E.

In certain embodiments there are only two R²′ substituents on Cycle-E.

In certain embodiments there are three R²′ substituents on Cycle-E.

In certain embodiments one R¹ substituent is halogen.

In certain embodiments two R¹ substituents are halogen.

In certain embodiments three R¹ substituents are halogen.

In certain embodiments one R² substituent is halogen.

In certain embodiments two R² substituents are halogen.

In certain embodiments three R² substituents are halogen.

In certain embodiments one R¹ substituent is haloalkyl.

In certain embodiments two R¹ substituents are haloalkyl.

In certain embodiments three R¹ substituents are haloalkyl.

In certain embodiments one R² substituent is haloalkyl.

In certain embodiments two R² substituents are haloalkyl.

In certain embodiments three R² substituents are haloalkyl.

In certain embodiments one R¹ substituent is alkyl.

In certain embodiments two R¹ substituents are alkyl.

In certain embodiments three R¹ substituents are alkyl.

In certain embodiments one R² substituent is alkyl.

In certain embodiments two R² substituents are alkyl.

In certain embodiments three R² substituents are alkyl.

In certain embodiments two R¹ groups are combined to form a fused phenyl ring.

In certain embodiments two R¹ groups are combined to form a fused 5-membered heteroaryl ring.

In certain embodiments two R¹ groups are combined to form a fused 6-membered heteroaryl ring.

In certain embodiments an R¹ group is combined with an R² group to form a fused 6-membered heterocycle.

In certain embodiments an R¹ group is combined with an R² group to form a fused 5-membered heterocycle.

In certain embodiments two R² groups are combined to form a fused phenyl ring.

In certain embodiments two R¹ groups are combined to form a fused phenyl ring.

In certain embodiments two R² groups are combined to form a fused 5-membered heteroaryl ring.

In certain embodiments two R² groups are combined to form a fused 6-membered heteroaryl ring.

In certain embodiments one R¹′ substituent is halogen.

In certain embodiments two R¹′ substituents are halogen.

In certain embodiments three R¹′ substituents are halogen.

In certain embodiments one R²′ substituent is halogen.

In certain embodiments two R²′ substituents are halogen.

In certain embodiments three R²′ substituents are halogen.

In certain embodiments one R¹′ substituent is haloalkyl.

In certain embodiments two R¹′ substituents are haloalkyl.

In certain embodiments three R¹′ substituents are haloalkyl.

In certain embodiments one R²′ substituent is haloalkyl.

In certain embodiments two R²′ substituents are haloalkyl.

In certain embodiments three R²′ substituents are haloalkyl.

In certain embodiments one R¹′ substituent is alkyl.

In certain embodiments two R¹′ substituents are alkyl.

In certain embodiments three R¹′ substituents are alkyl.

In certain embodiments one R²′ substituent is alkyl.

In certain embodiments two R²′ substituents are alkyl.

In certain embodiments three R²′ substituents are alkyl.

In certain embodiments two R¹′ groups are combined to form a fused phenyl ring.

In certain embodiments two R¹′ groups are combined to form a fused 5-membered heteroaryl ring.

In certain embodiments two R¹′ groups are combined to form a fused 6-membered heteroaryl ring.

In certain embodiments an R¹′ group is combined with an R² group to form a fused 6-membered heterocycle.

In certain embodiments an R¹′ group is combined with an R² group to form a fused 5-membered heterocycle.

In certain embodiments two R²′ groups are combined to form a fused phenyl ring.

In certain embodiments two R¹′ groups are combined to form a fused phenyl ring.

In certain embodiments two R²′ groups are combined to form a fused 5-membered heteroaryl ring.

In certain embodiments two R²′ groups are combined to form a fused 6-membered heteroaryl ring.

In certain embodiments, one R¹, R², or R¹′ is selected from:

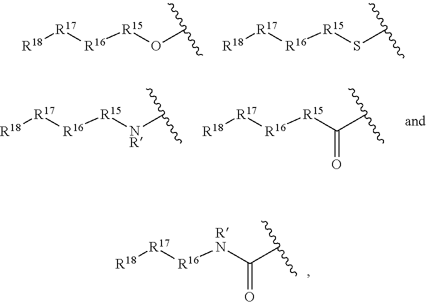

wherein each R′ is independently selected from hydrogen, alkyl, haloalkyl, aryl, heterocycle, and heteroaryl.

In certain embodiments, R¹, R², or R¹′ are a heterocycle group optionally substituted with 1 or 2 substituents selected from R′.

In certain embodiments, R¹, R², or R¹′ are is a 6-membered heterocycle group with one or two nitrogen atoms.

In certain embodiments, R¹, R², or R¹′ are is a 6-membered heterocycle group with one or two oxygen atoms

In certain embodiments, one R¹, R², or R¹′ is selected from:

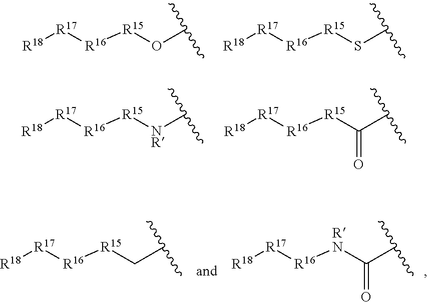

wherein each R′ is independently selected from hydrogen, alkyl, haloalkyl, aryl, heterocycle, and heteroaryl.

In certain embodiments, R¹ is a heterocycle group optionally substituted with 1 or 2 substituents selected from R′.

In certain embodiments, R¹ is a 6-membered heterocycle group with one or two nitrogen atoms.

In certain embodiments, R¹ is a 6-membered heterocycle group with one or two oxygen atoms

In certain embodiments, R² is a heterocycle group optionally substituted with 1 or 2 substituents selected from R′.

In certain embodiments, R² is a 6-membered heterocycle group with one or two nitrogen atoms.

In certain embodiments, R² is a 6-membered heterocycle group with one or two oxygen atoms

In certain embodiments, R^{1′} is a heterocycle group optionally substituted with 1 or 2 substituents selected from R′.

In certain embodiments, R¹′ is a 6-membered heterocycle group with one or two nitrogen atoms.

In certain embodiments, R^{1′} is a 6-membered heterocycle group with one or two oxygen atoms

In certain embodiments, one R¹, R², or R¹′ is selected from:

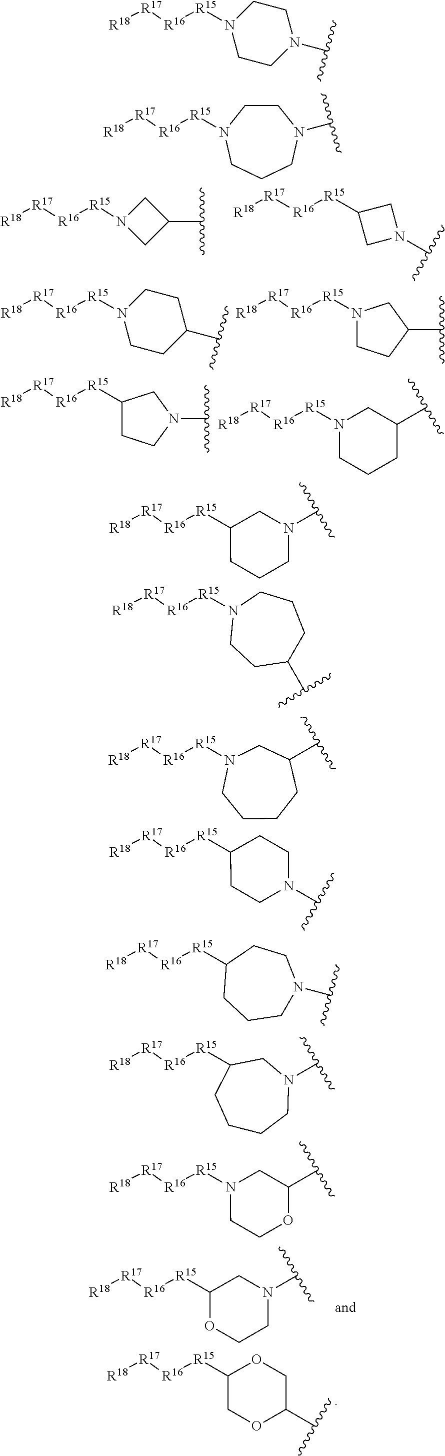

In certain embodiments, one R¹, R², R¹′ is selected from:

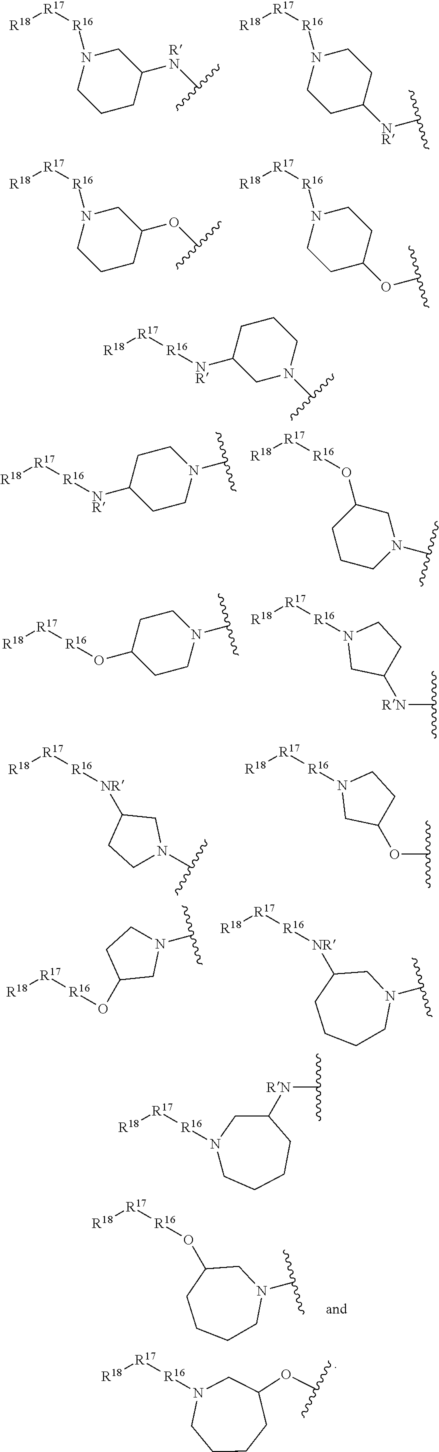

In certain embodiments, one R¹, R², R¹′ is selected from:

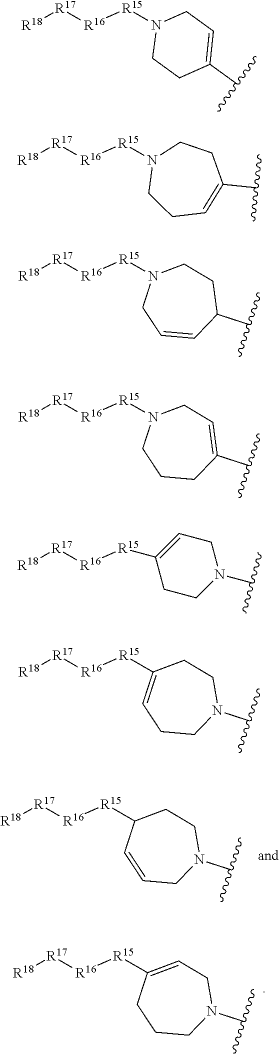

In certain embodiments, one R¹, R², R¹′ is selected from:

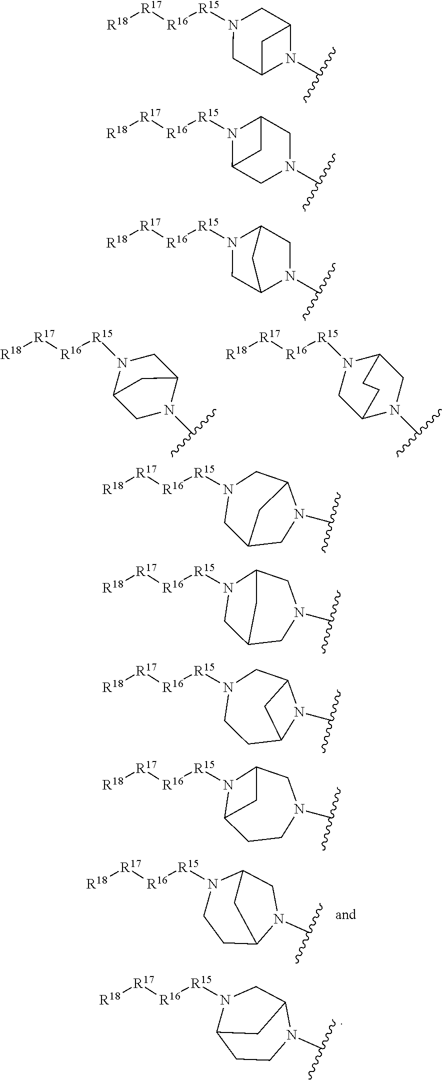

In certain embodiments, one R¹, R², or R¹′ is selected from:

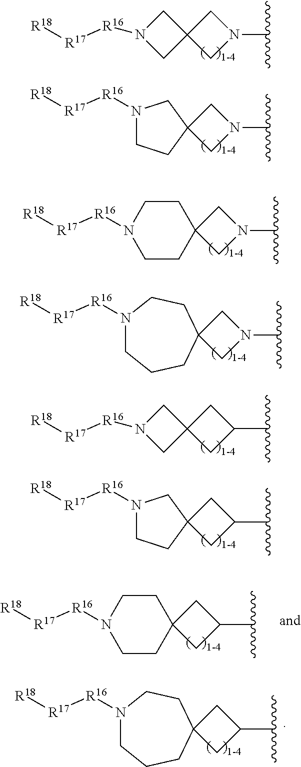

In certain embodiments, one R¹, R², or R¹′ is selected from:

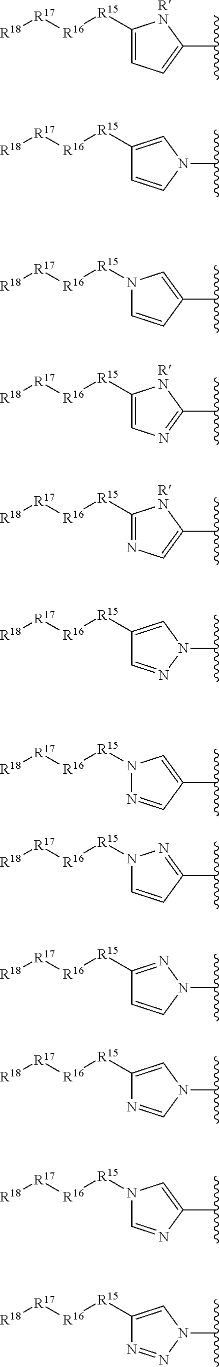

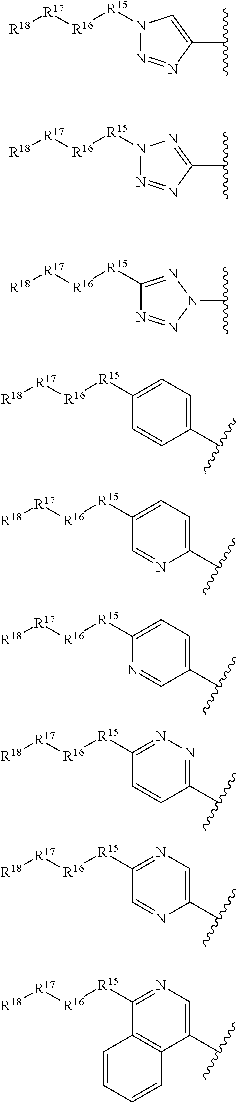

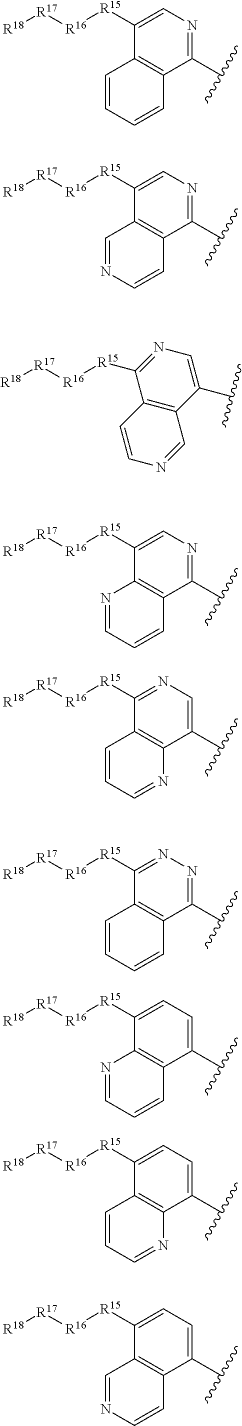

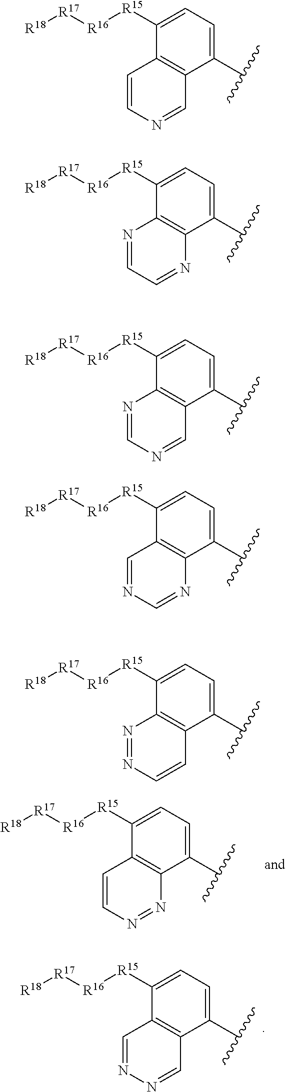

In certain embodiments, one R¹, R², or R¹′ is selected from:

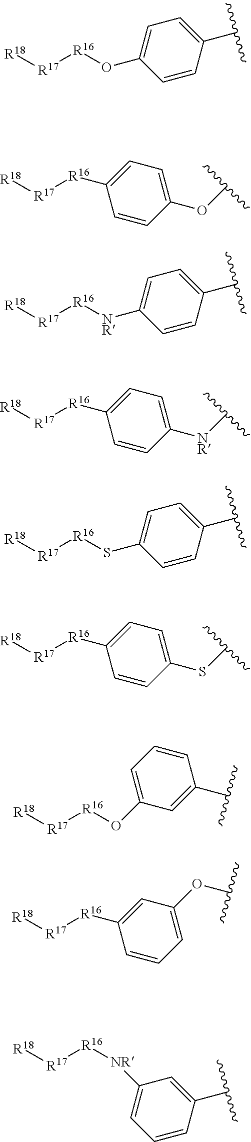

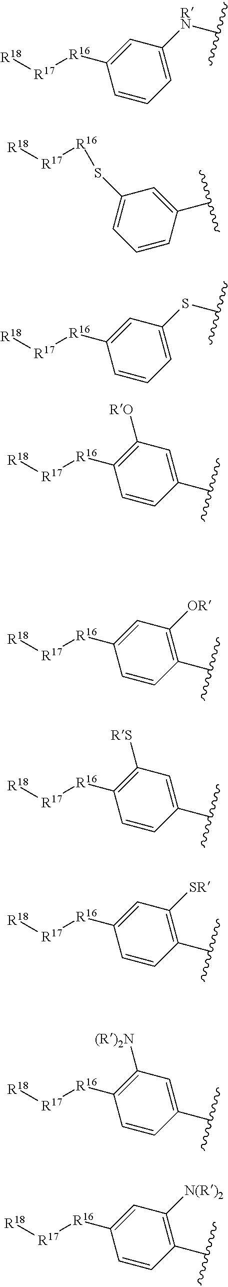

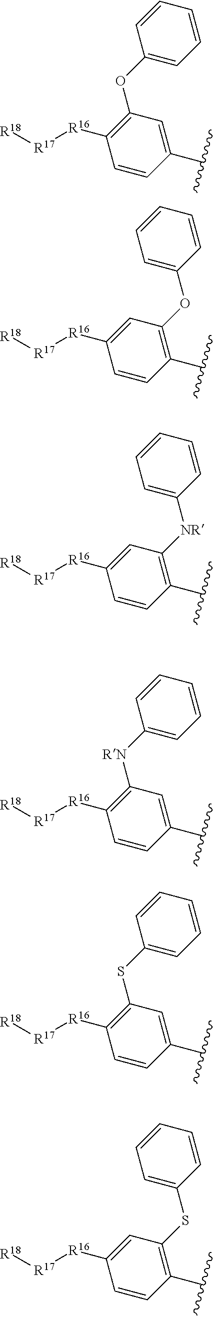

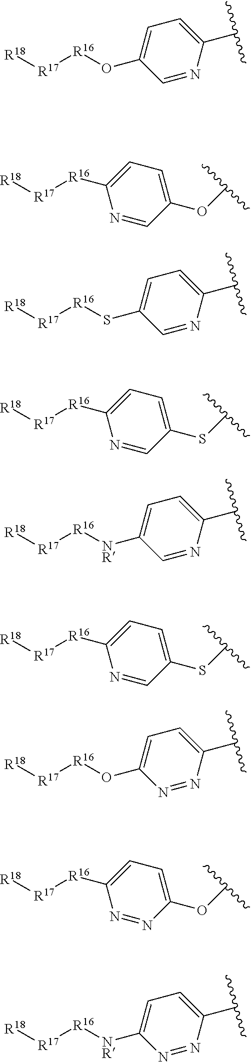

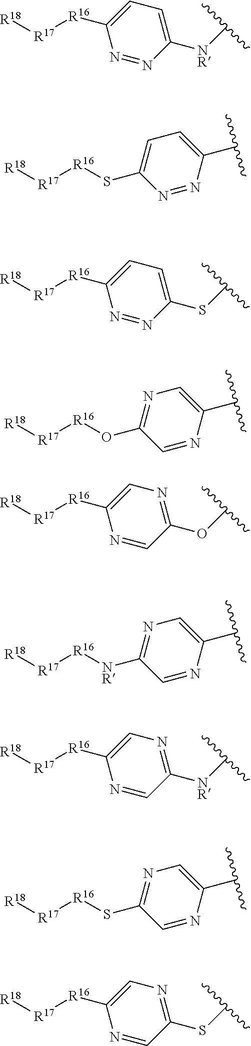

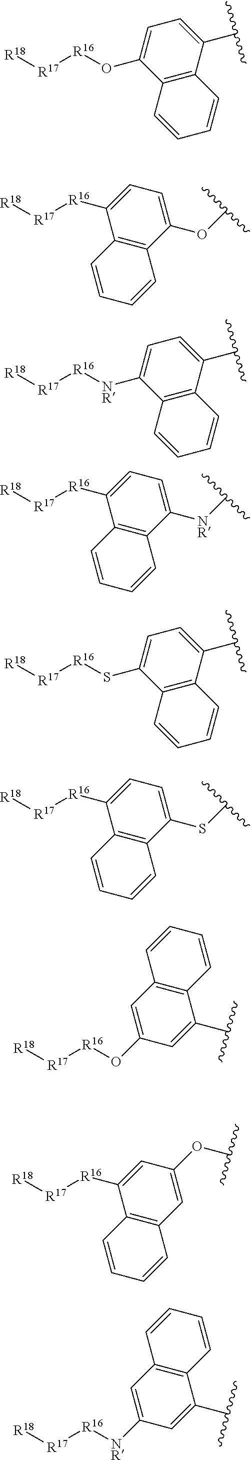

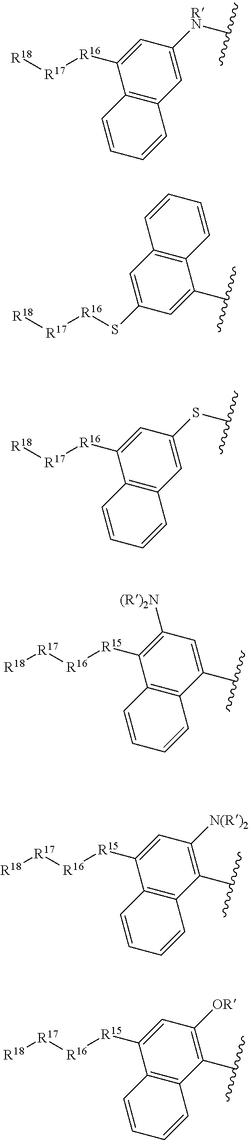

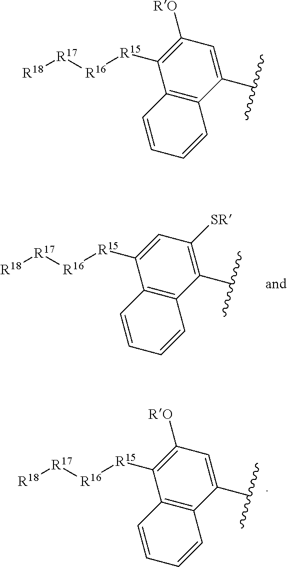

In certain embodiments, one R¹, R², or R¹′ is selected from:

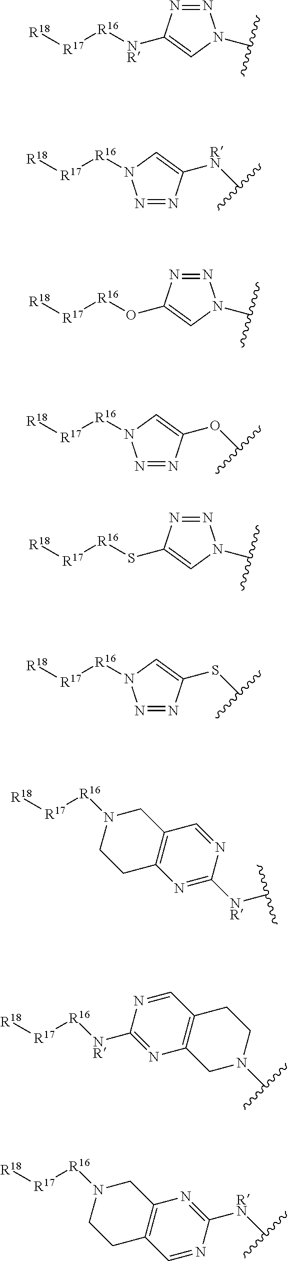

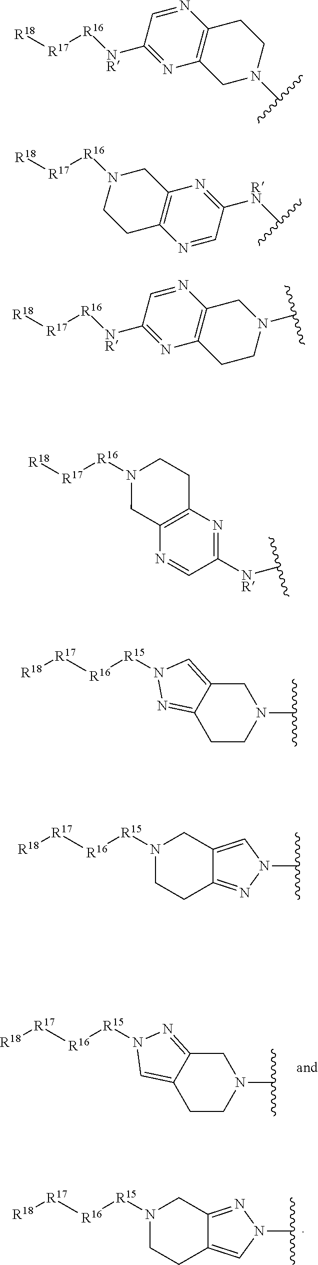

In certain embodiments, one R¹, R² or R¹′ is selected from:

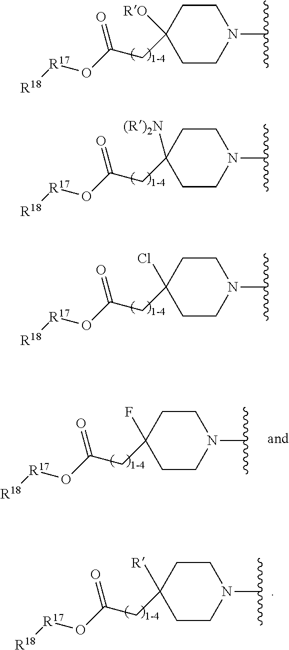

In certain embodiments, one R¹, R², or R^{1′} is selected from:

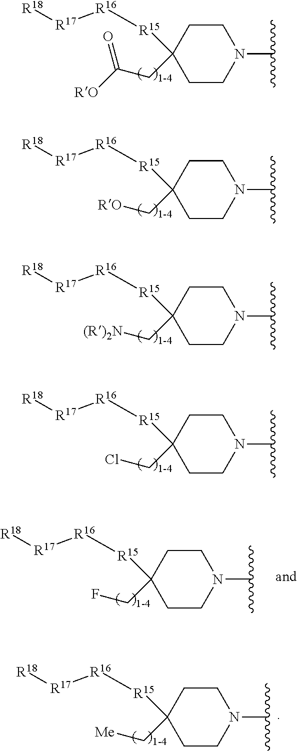

In certain embodiments, one R¹, R², or R¹′ is selected from:

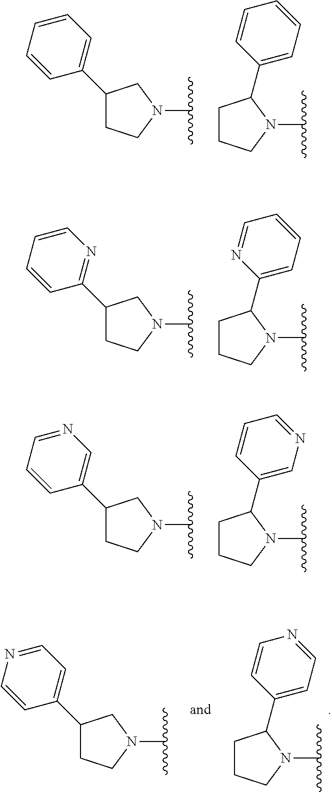

In certain embodiments, one R¹, R², or R¹′ is selected from:

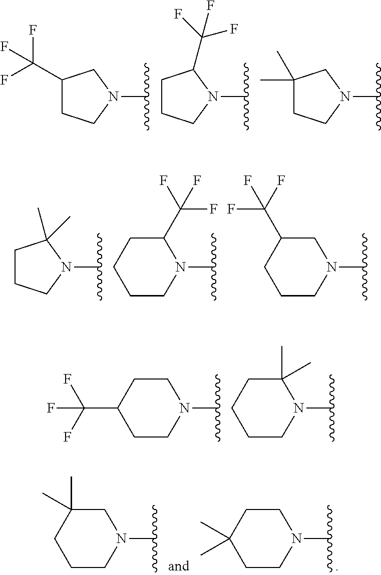

In certain embodiments, one R¹, R² or R¹′ is selected from:

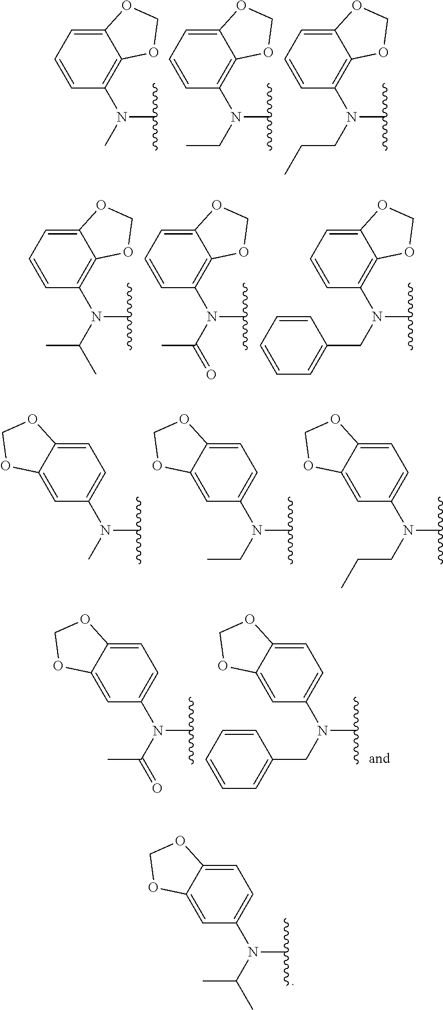

In certain embodiments, one R¹, R², or R¹′ is selected from:

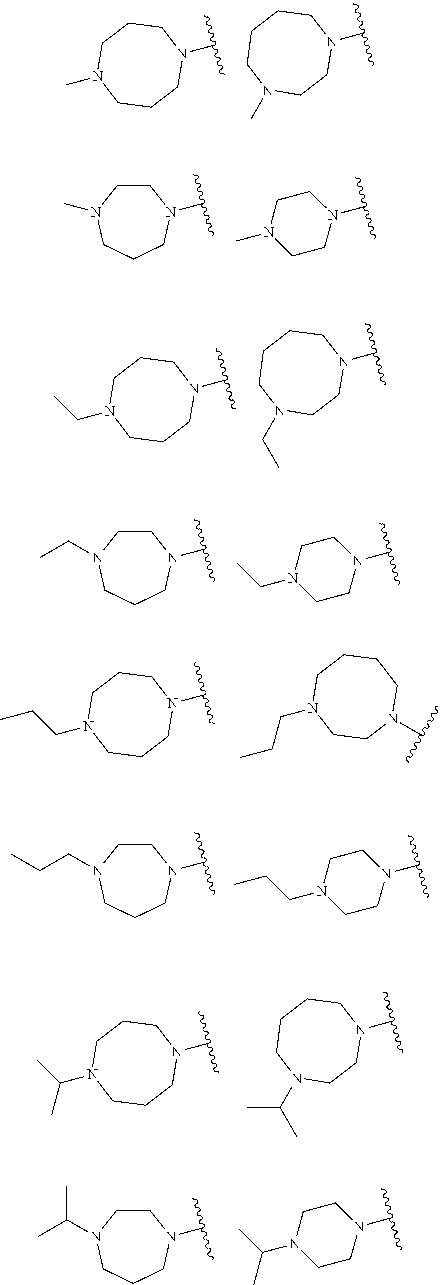

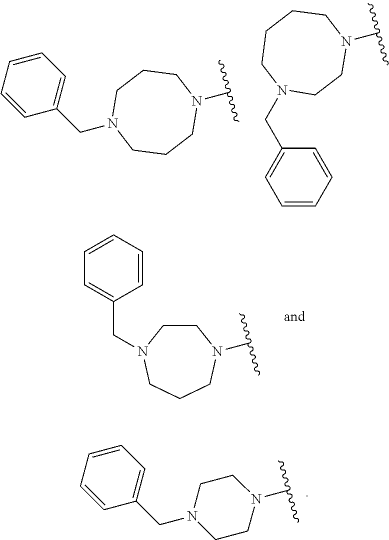

In certain embodiments, one R¹, R², or R¹′ is selected from:

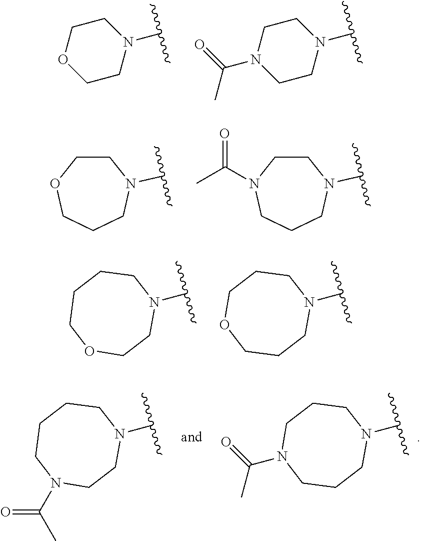

In certain embodiments, one R¹, R², or R¹′ is selected from:

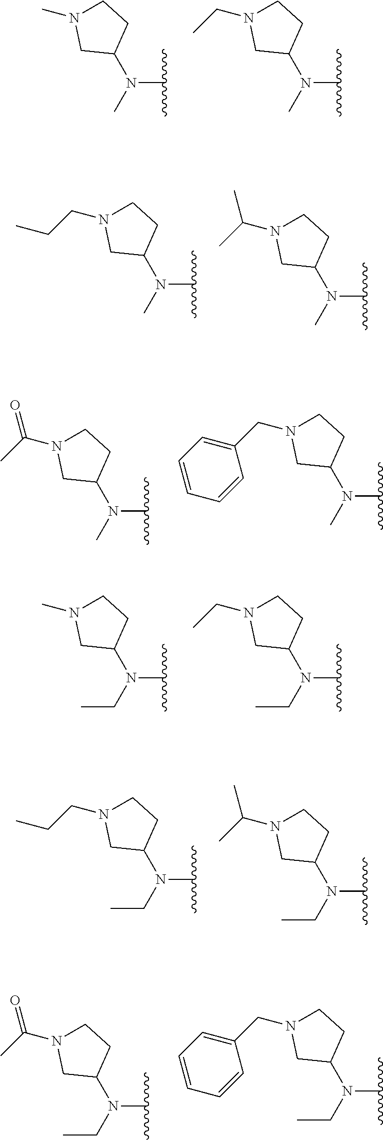

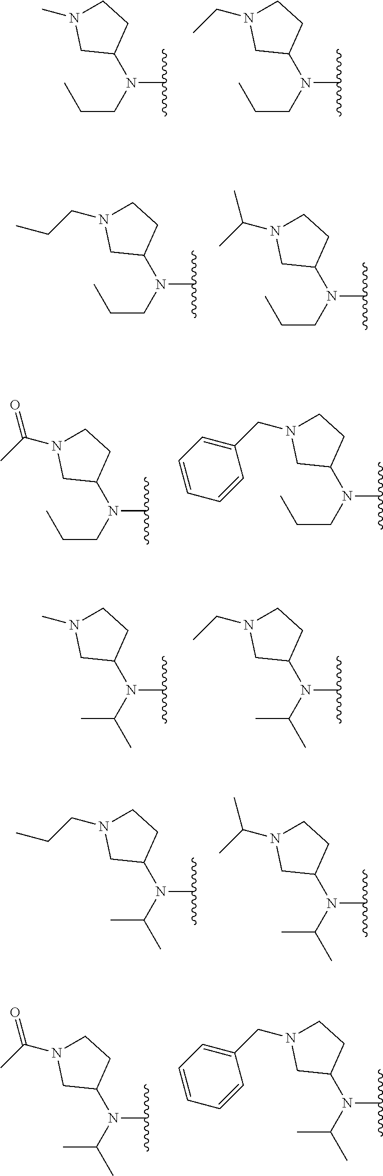

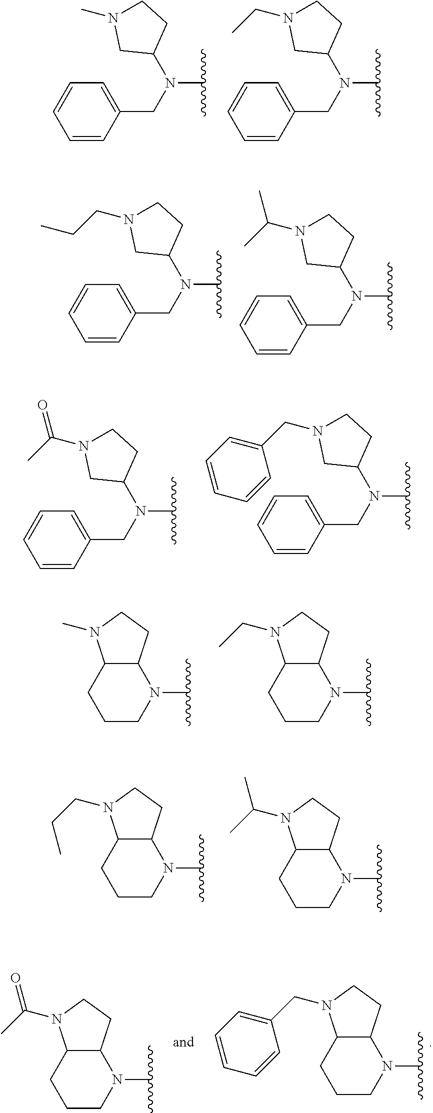

In certain embodiments, one R¹, R², or R^{1′} is selected from:

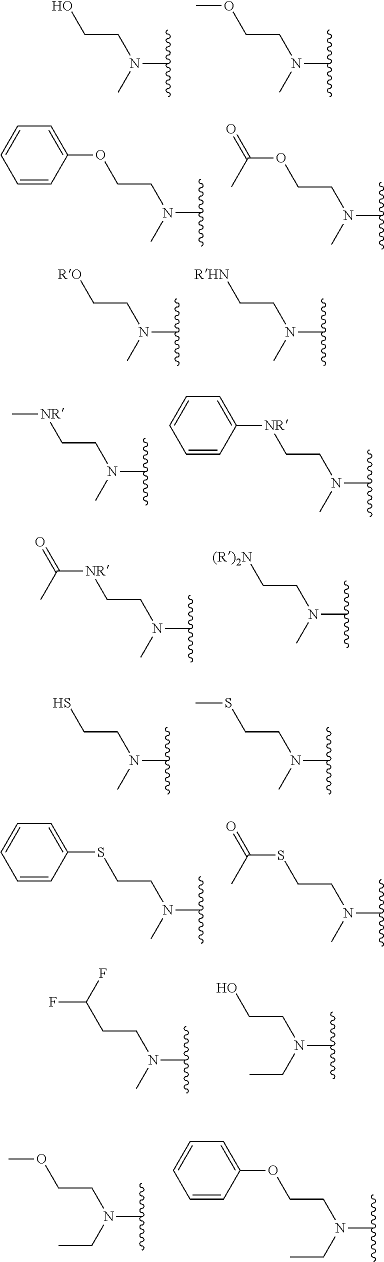

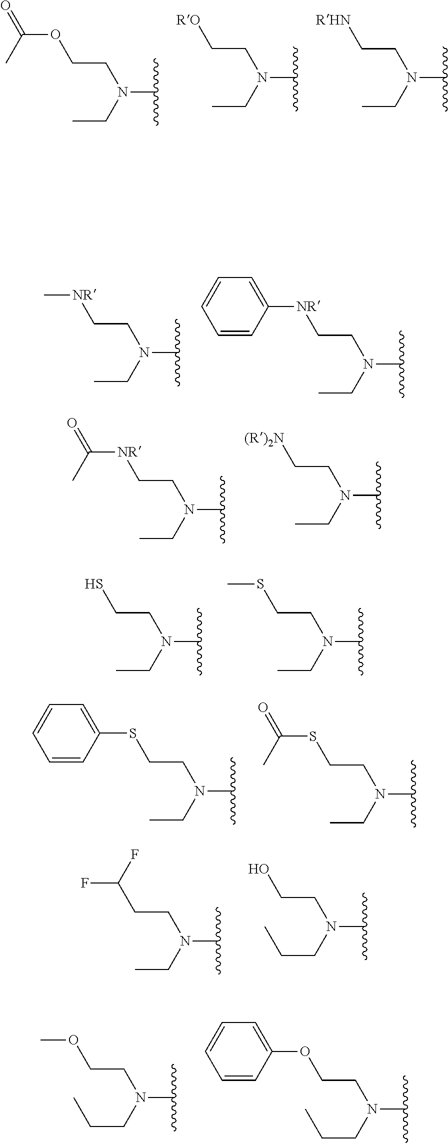

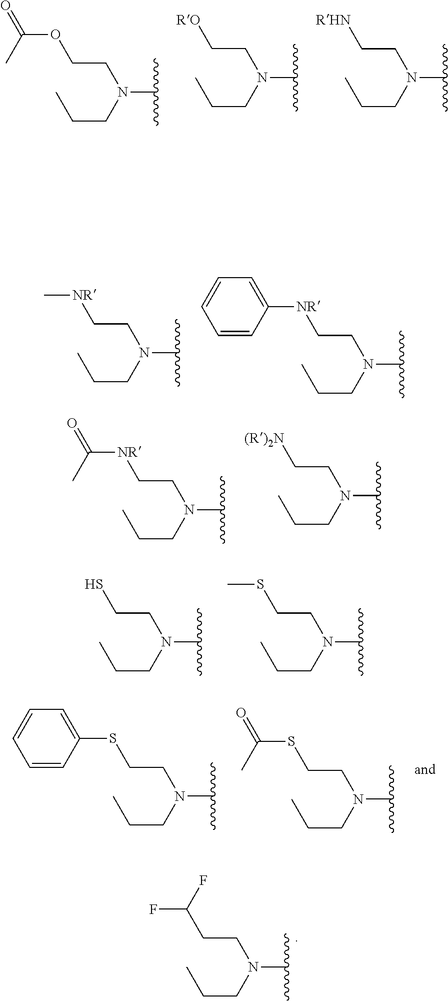

In certain embodiments, one R¹, R², or R^{1′} is selected from:

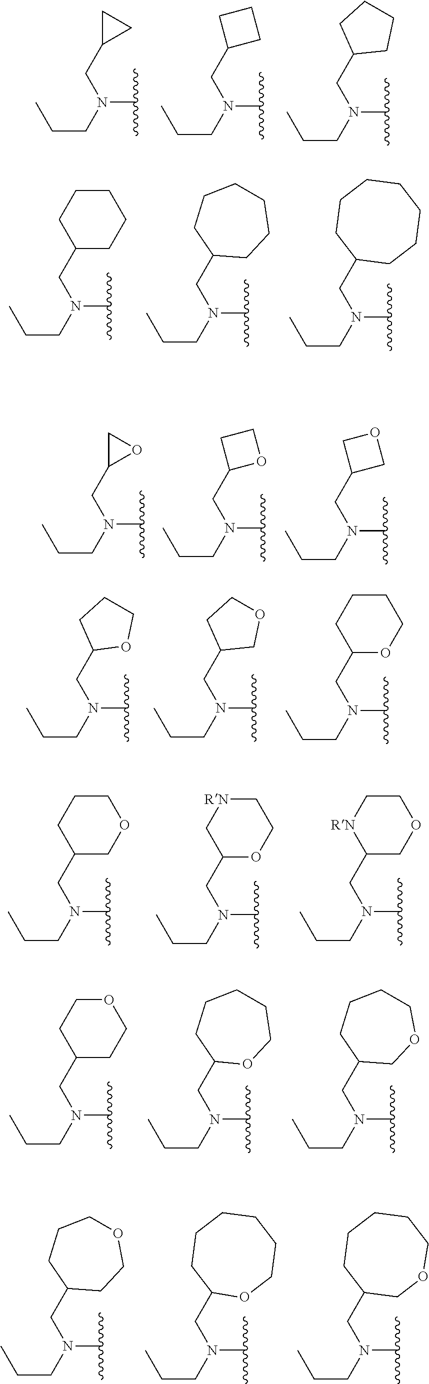

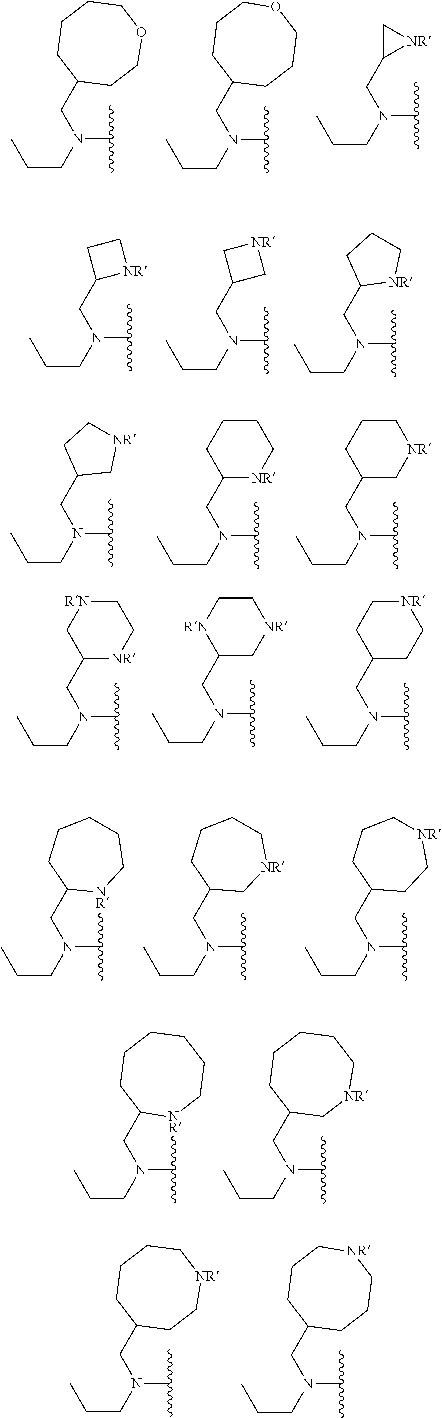

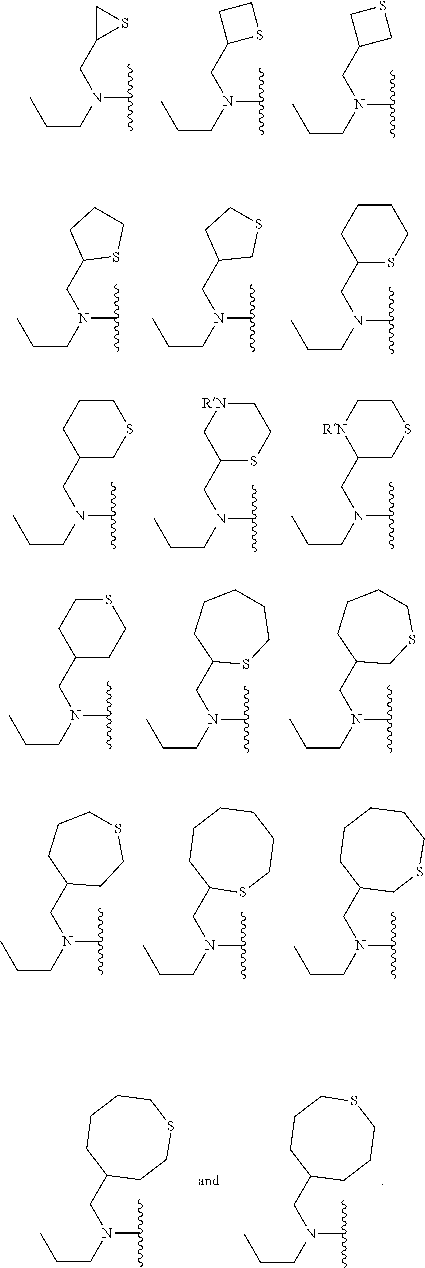

In certain embodiments, one R¹, R², or R¹′ is selected from:

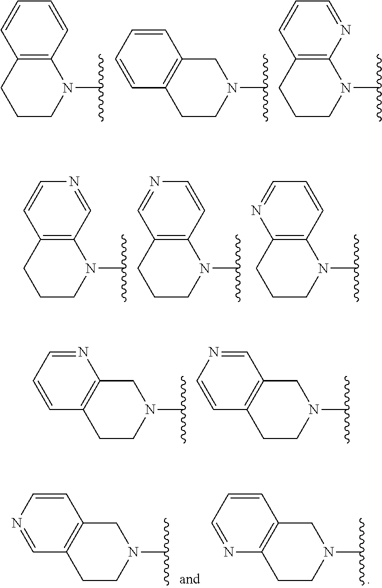

In certain embodiments, one R¹, R², or R¹′ is selected from:

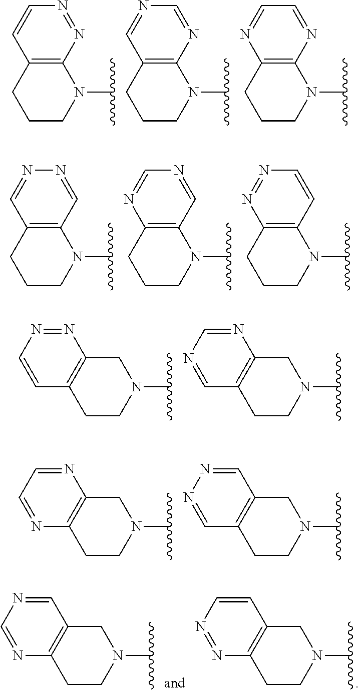

In certain embodiments, one R¹, R², or R¹′ is selected from:

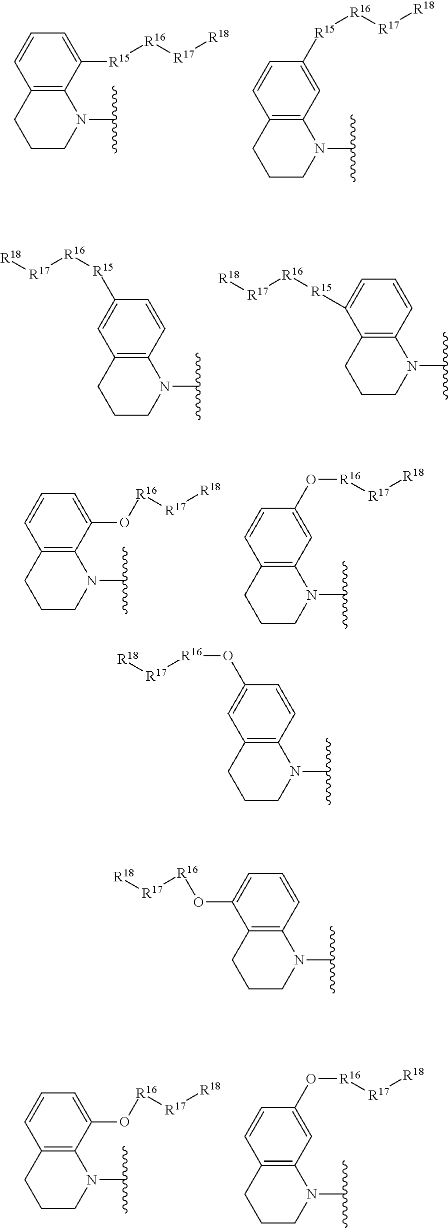

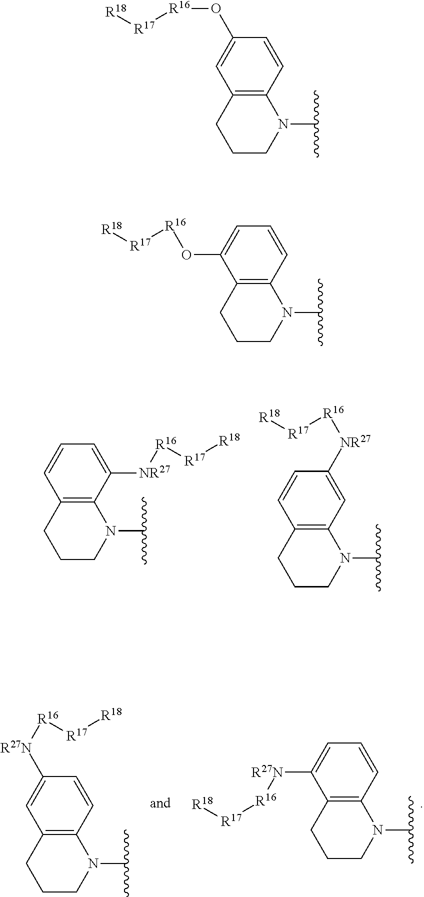

In certain embodiments, one R¹, R², or R¹′ is selected from:

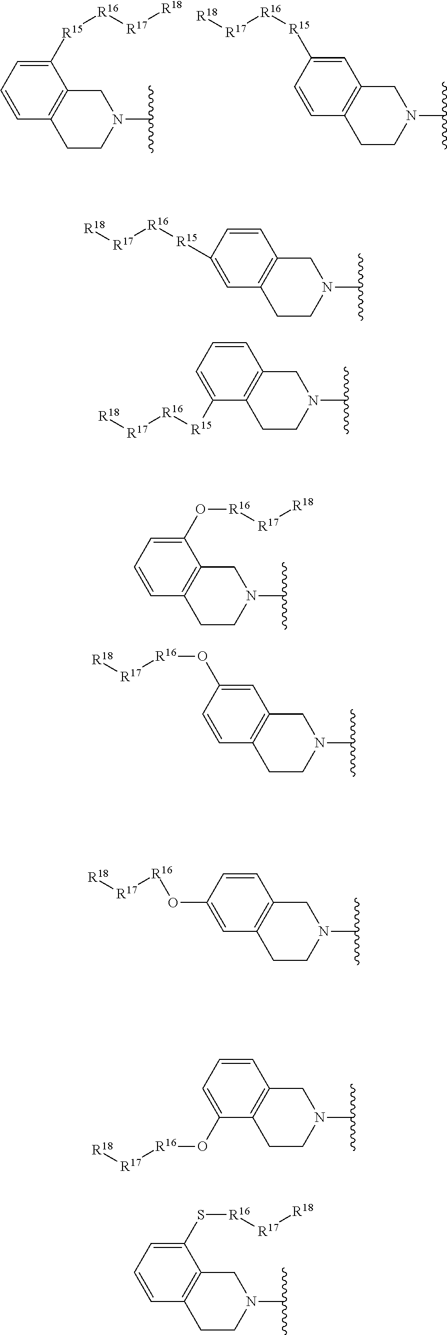

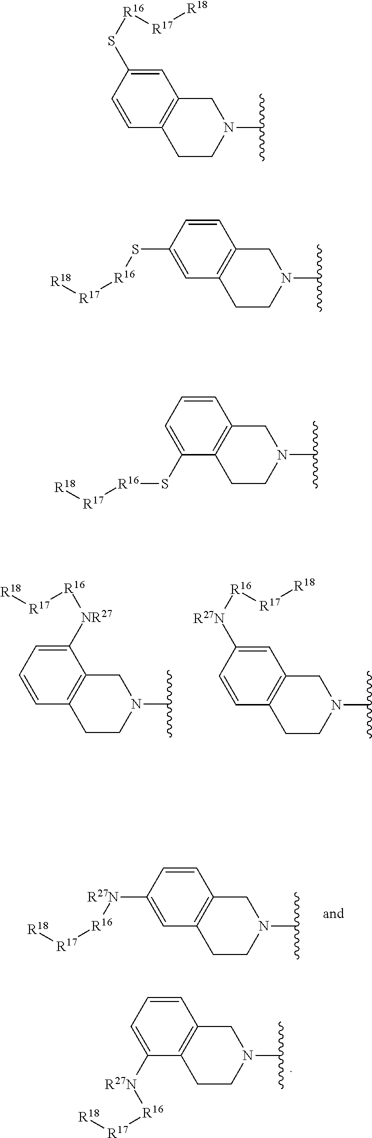

In certain embodiments, one R¹, R², or R¹′ is selected from:

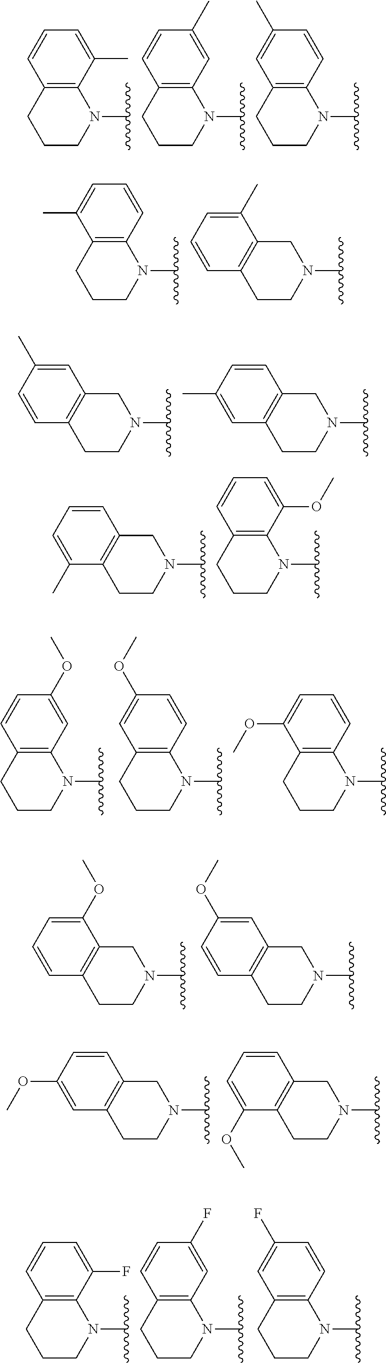

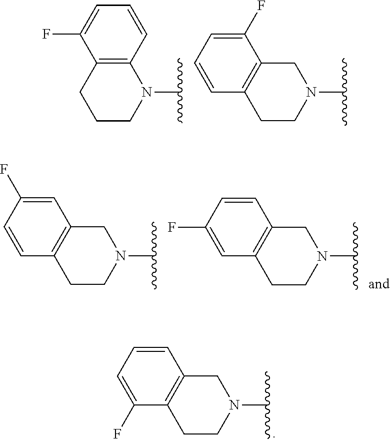

In certain embodiments, one R¹, R², or R¹′ is selected from:

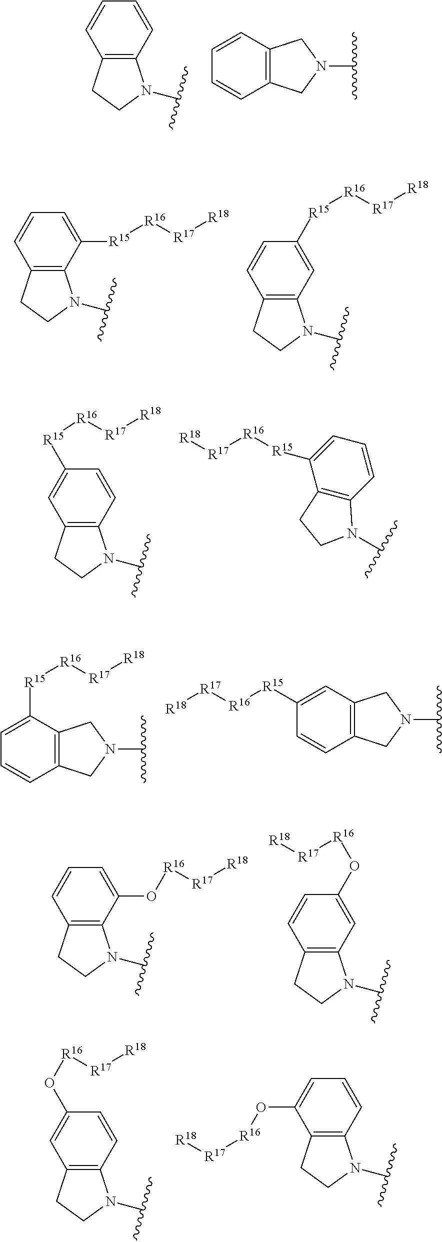

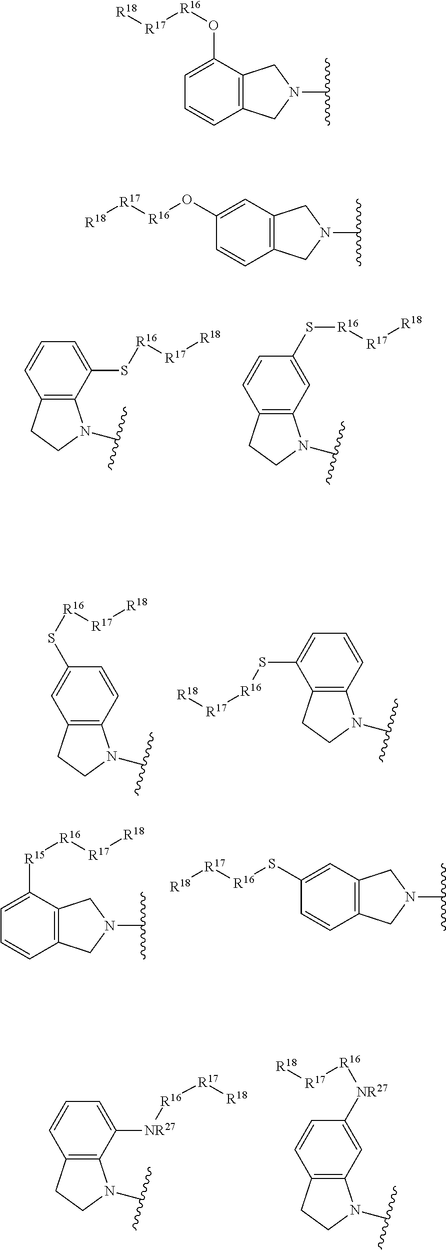

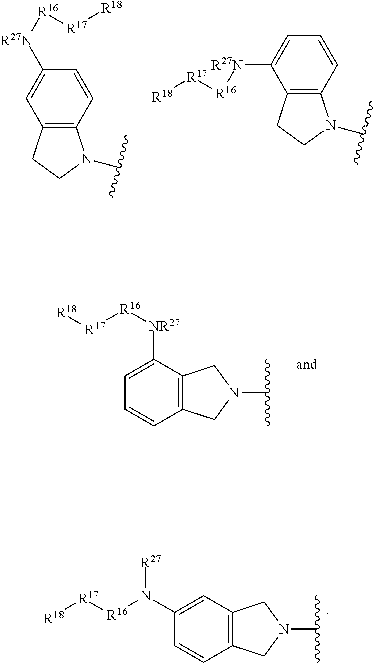

In certain embodiments, one R¹, R², or R¹′ is selected from:

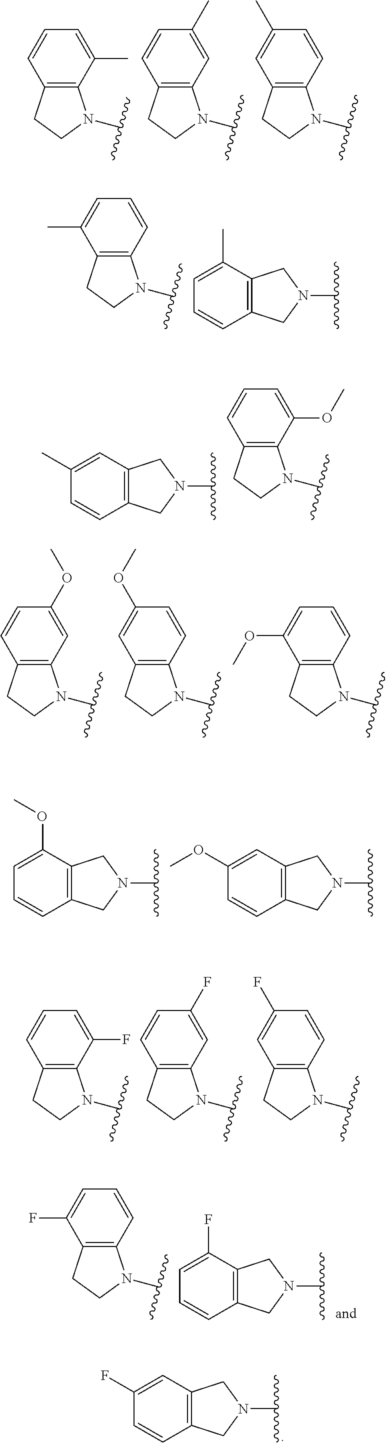

In certain embodiments, one R¹, R², or R¹′ is selected from:

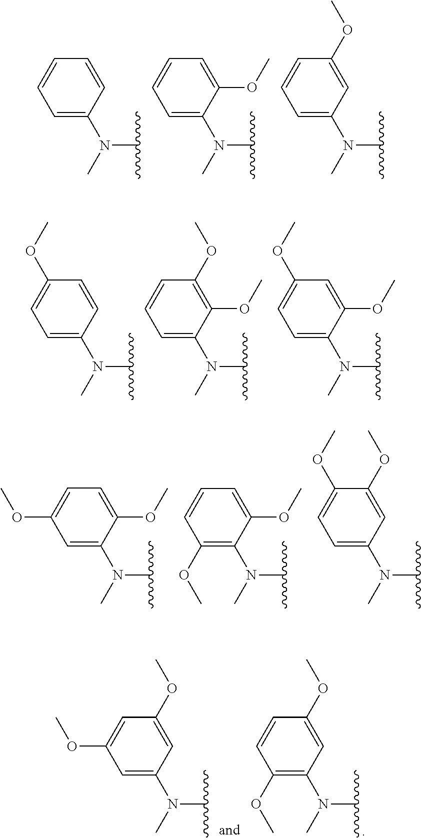

In certain embodiments, one R¹, R², or R¹′ is selected from:

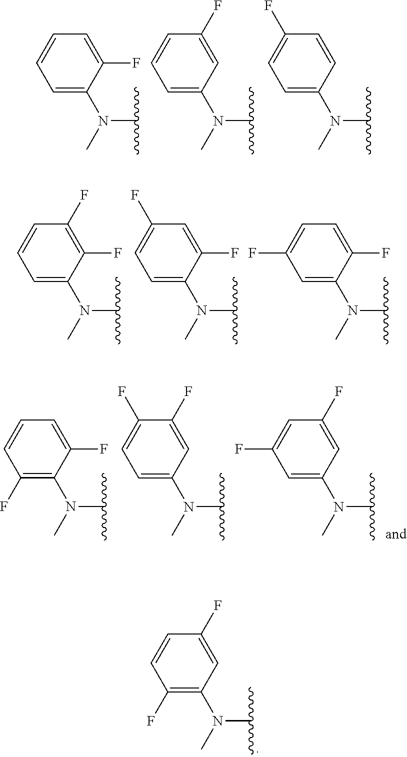

In certain embodiments, one R¹, R², or R¹′ is selected from:

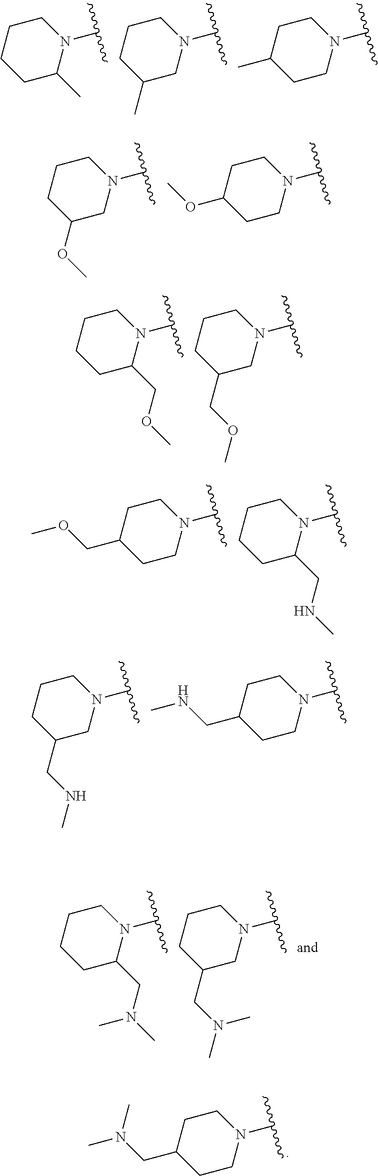

In certain embodiments, one R¹, R², or R¹′ is selected from:

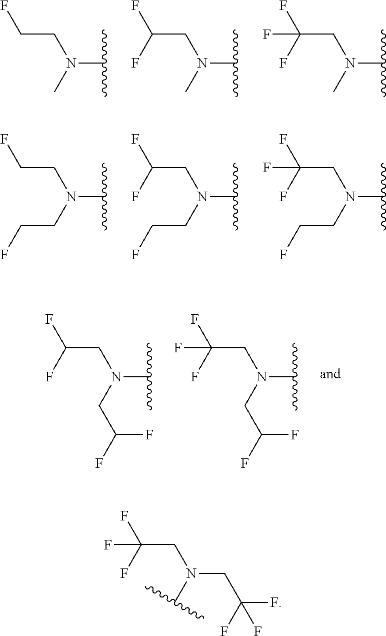

In certain embodiments, one R¹, R², or R¹′ is selected from:

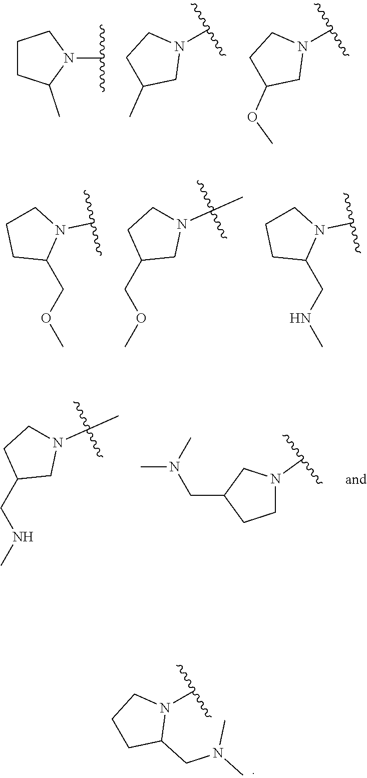

In certain embodiments, one R¹, R², or R¹′ is selected from:

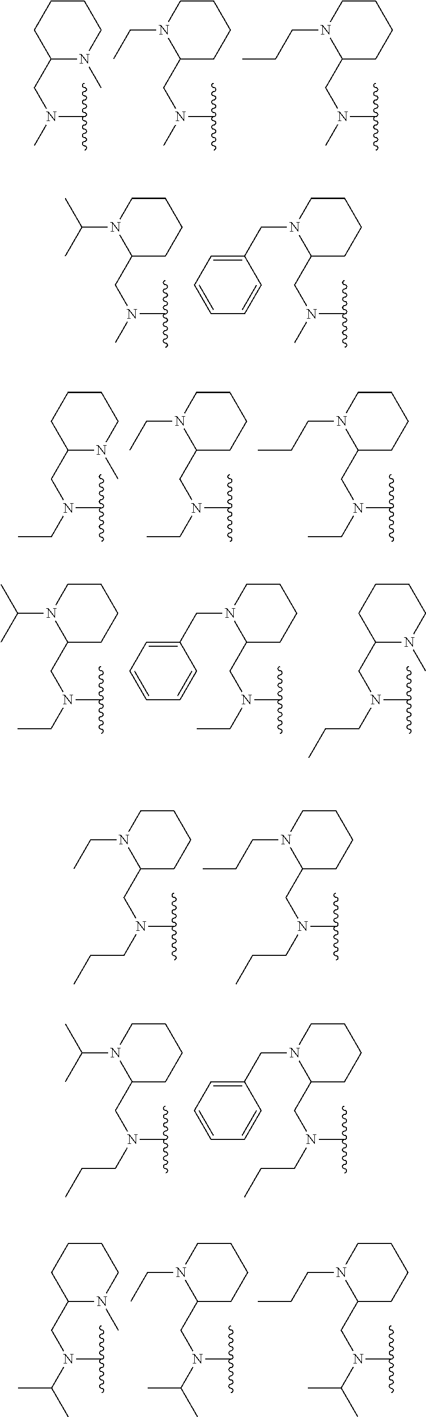

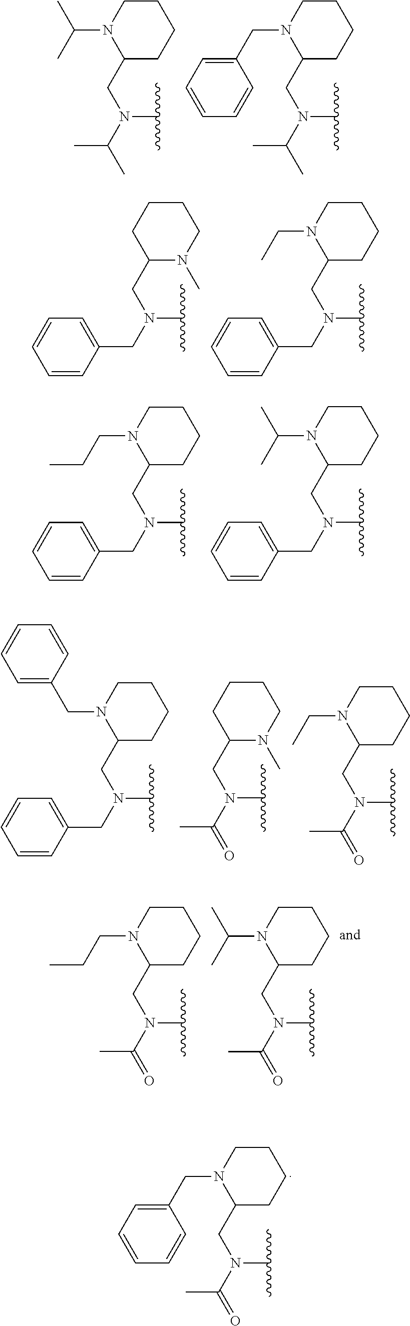

In certain embodiments, one R¹, R², or R¹′ is selected from:

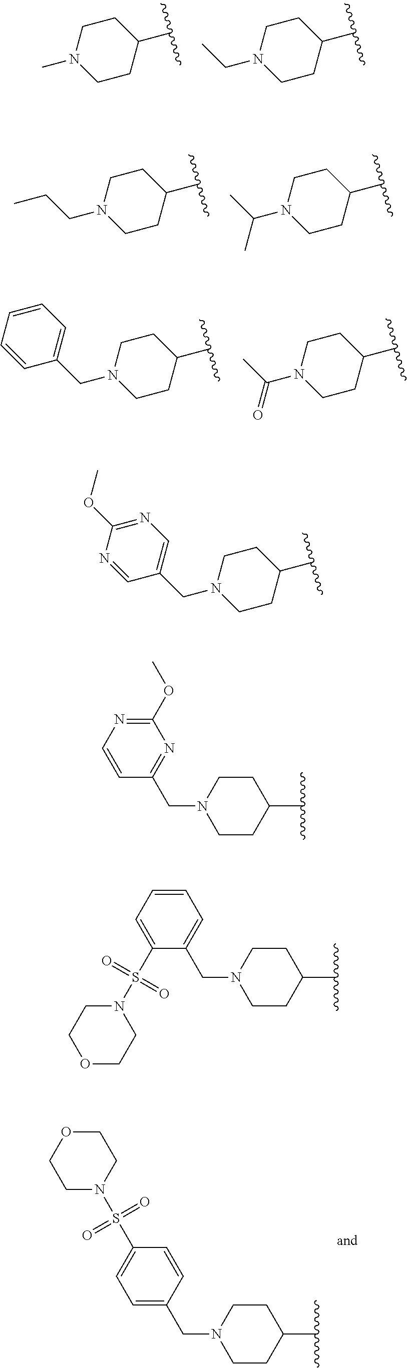

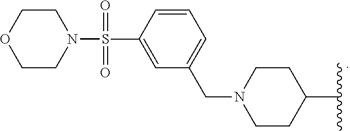 [SMILES: *C1CCN(Cc2cccc(S(=O)(=O)N3CCOCC3)c2)CC1]

In certain embodiments, one R¹, R², or R¹′ is selected from:

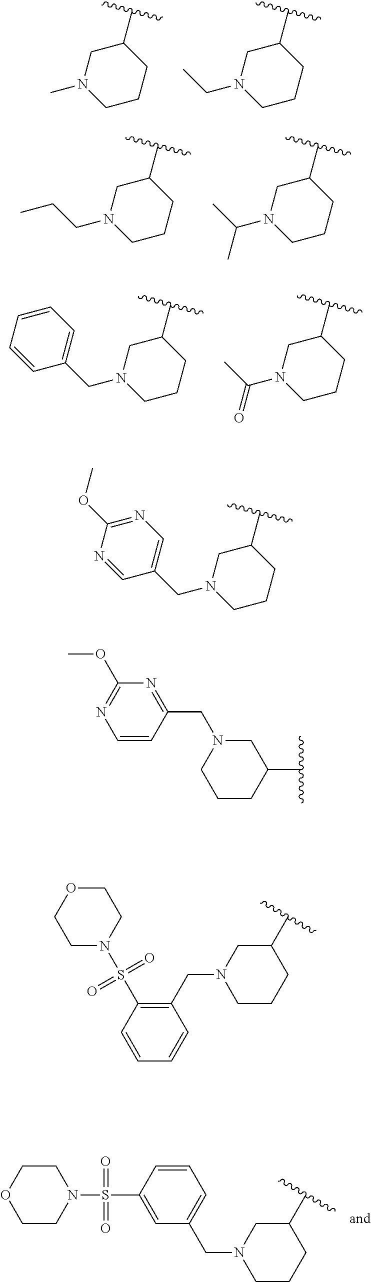

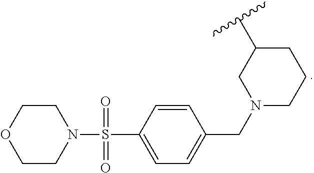 [SMILES: *C1CCCN(Cc2ccc(S(=O)(=O)N3CCOCC3)cc2)C1]

In certain embodiments, one R¹, R², or R¹′ is selected from:

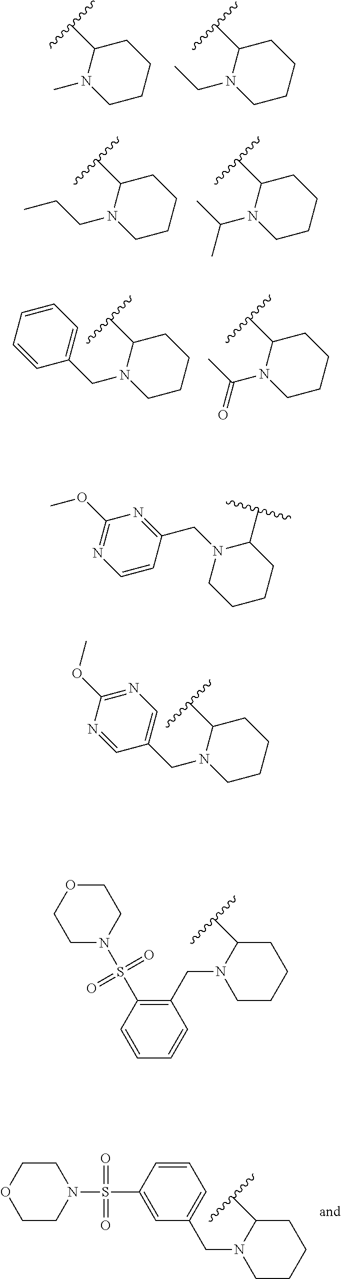

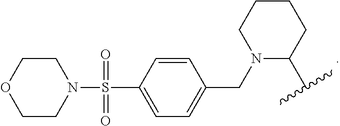 [SMILES: *C1CCCCN1Cc1ccc(S(=O)(=O)N2CCOCC2)cc1]

In certain embodiments, one R¹, R², or R¹′ is selected from:

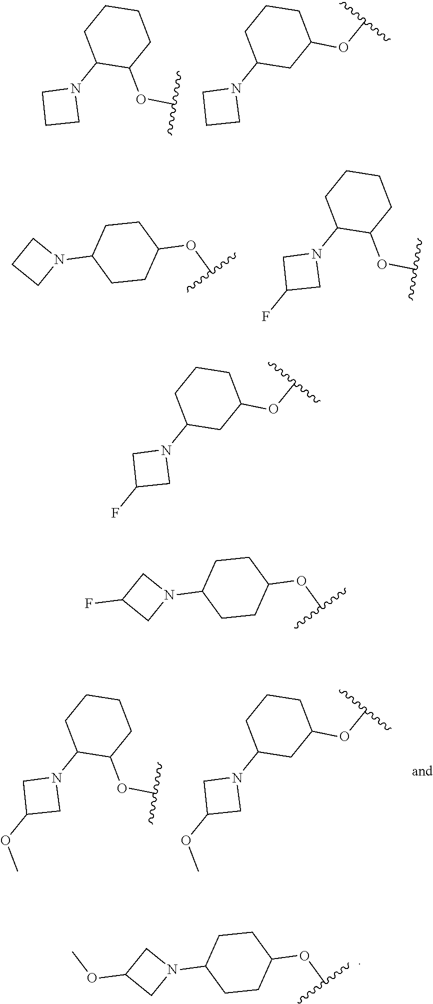

In certain embodiments, one R, R² or R¹′ is selected from:

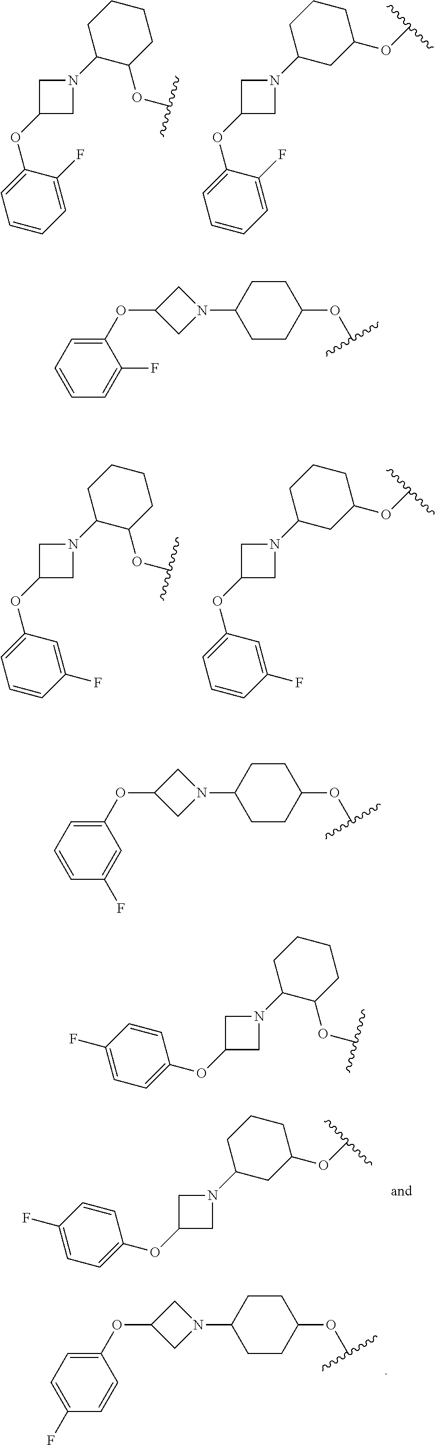

In certain embodiments, one R¹, R², or R¹′ is selected from:

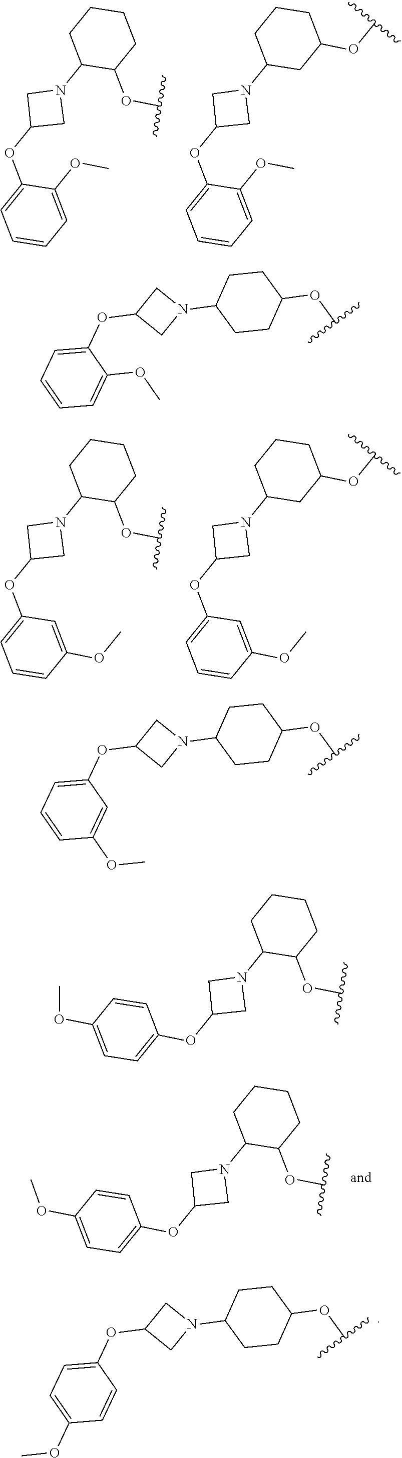

In certain embodiments, one R¹, R², or R¹′ is selected from:

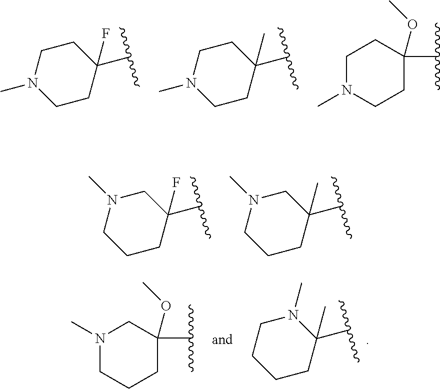

In certain embodiments, one R¹, R², or R¹′ is selected from:

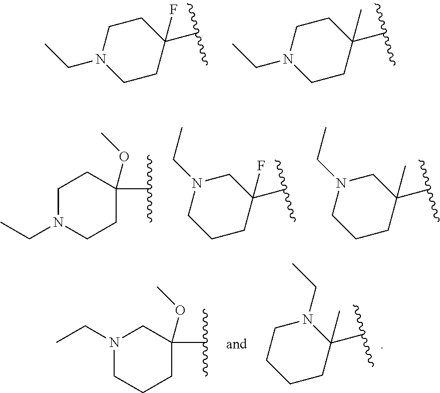

In certain embodiments, one R¹, R², or R¹′ is selected from:

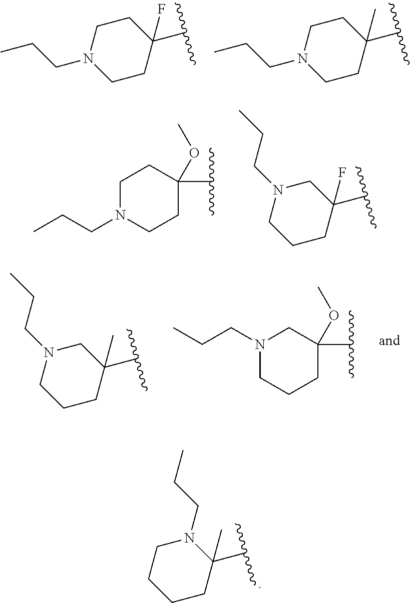

In certain embodiments, one R¹, R², or R¹′ is selected from:

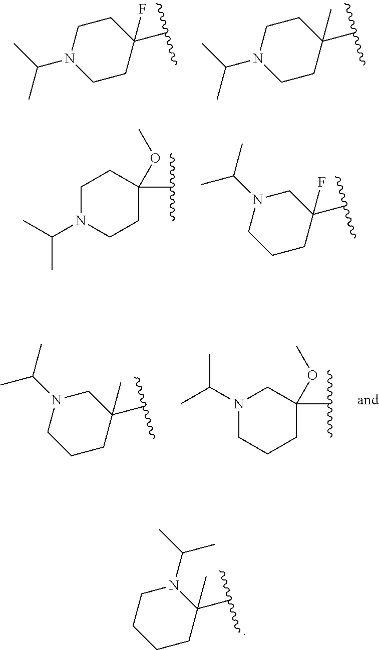

In certain embodiments, one R¹, R², or R¹′ is selected from:

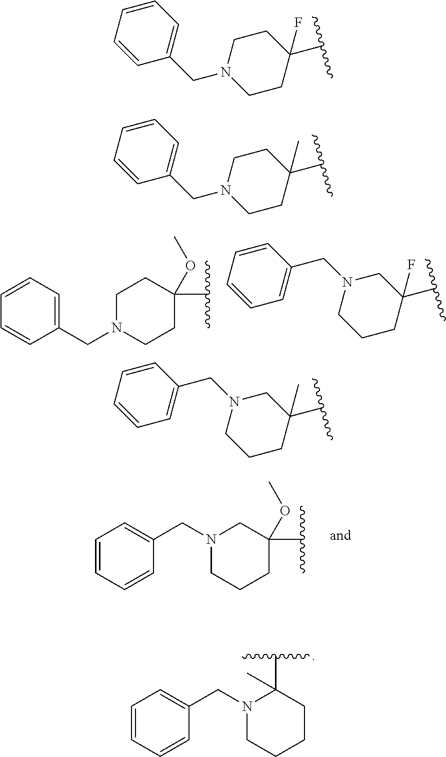

In certain embodiments, one R¹, R², or R¹′ is selected from:

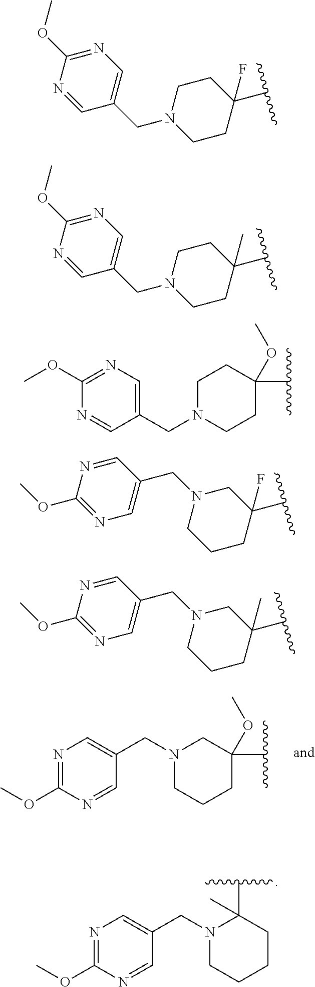

In certain embodiments, one R¹, R², or R¹′ is selected from:

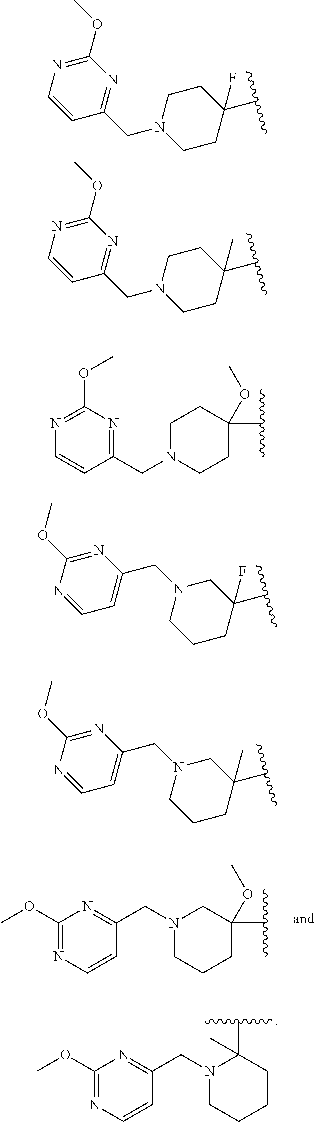

In certain embodiments, one R¹, R², or R¹′ is selected from:

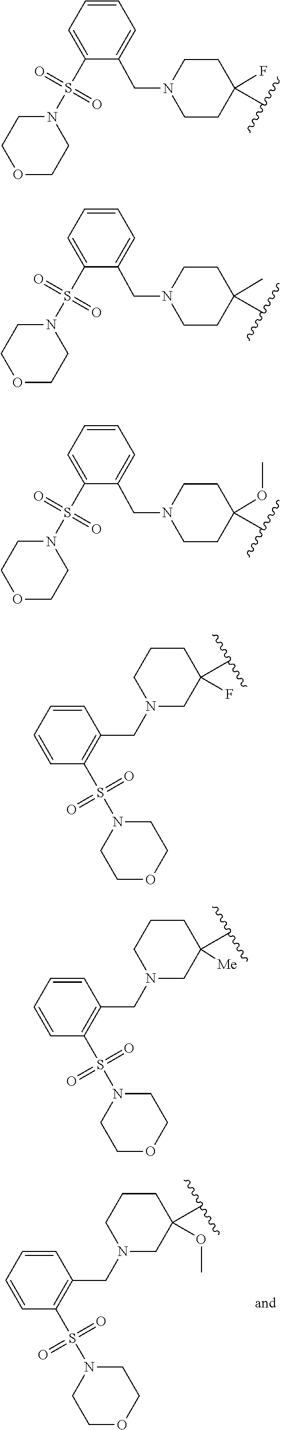

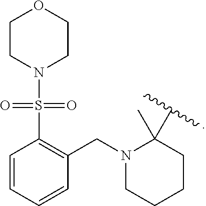 [SMILES: *C1(C)CCCCN1Cc1ccccc1S(=O)(=O)N1CCOCC1]

In certain embodiments, one R¹, R², or R^{1′} is selected from:

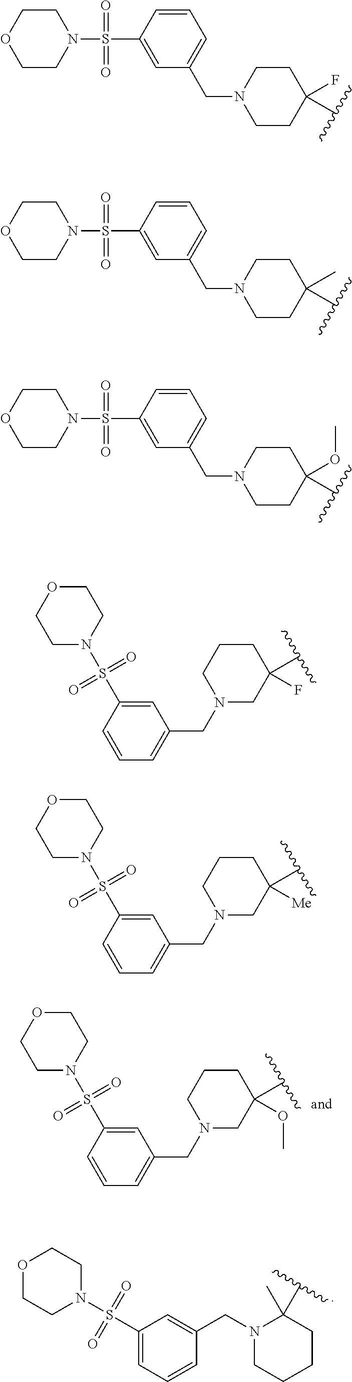

In certain embodiments, one R¹, R², or R¹′ is selected from:

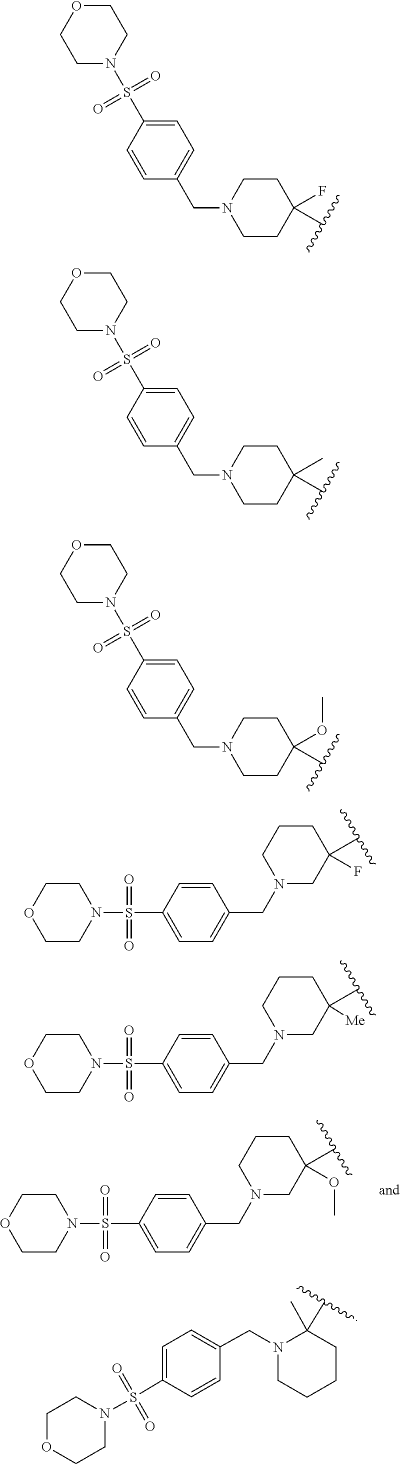

In certain embodiments, one R¹, R², or R¹′ is selected from:

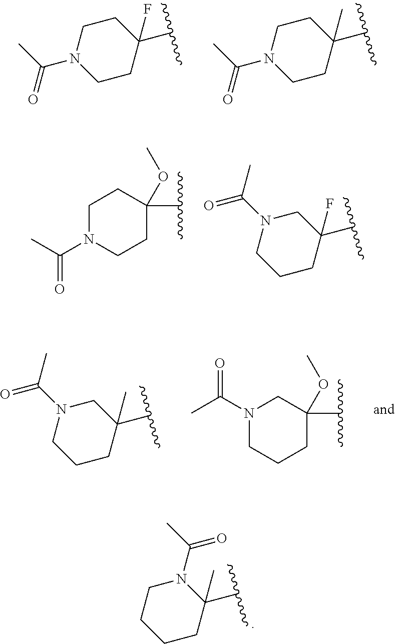

In certain embodiments, one R¹, R², or R¹′ is selected from:

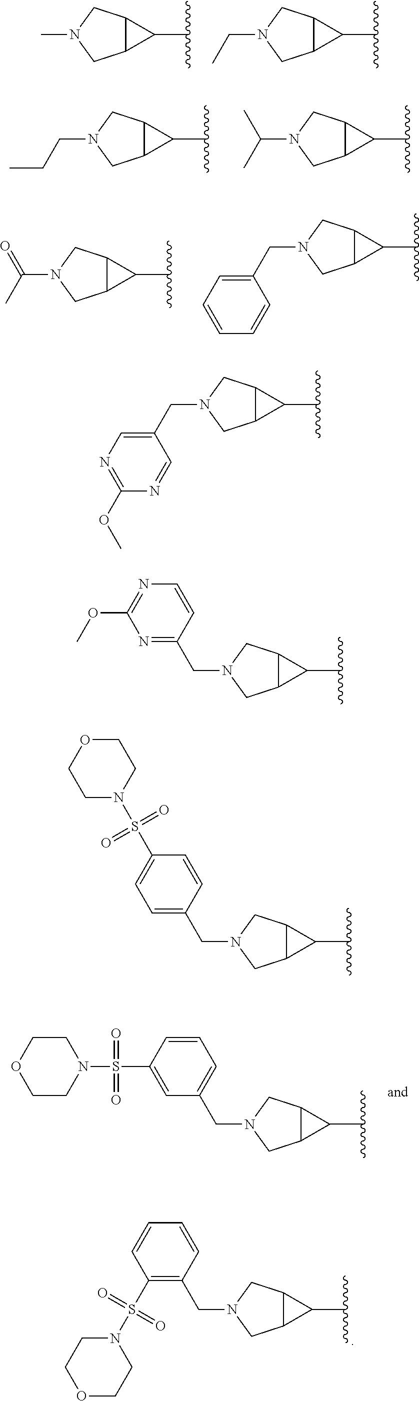

In certain embodiments, one R¹, R², or R¹′ is selected from:

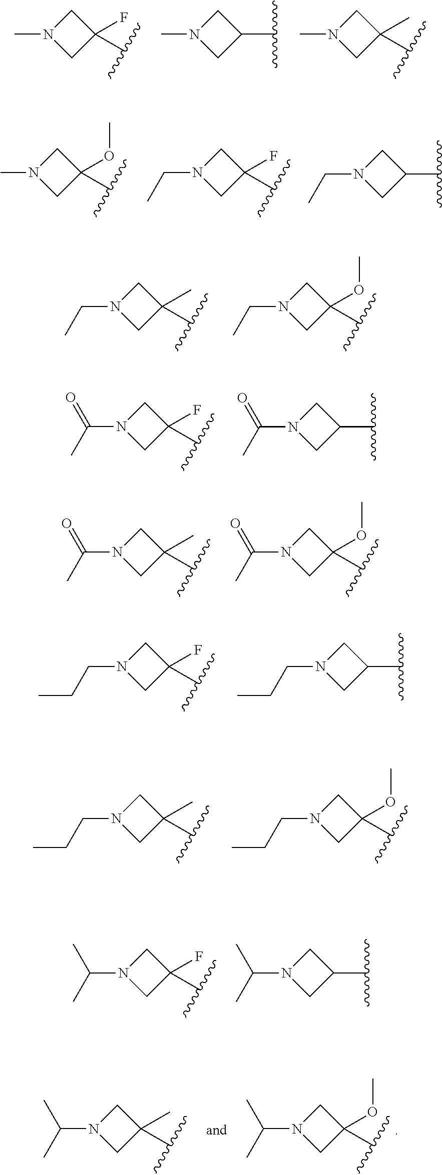

In certain embodiments, one R¹, R², or R¹′ is selected from:

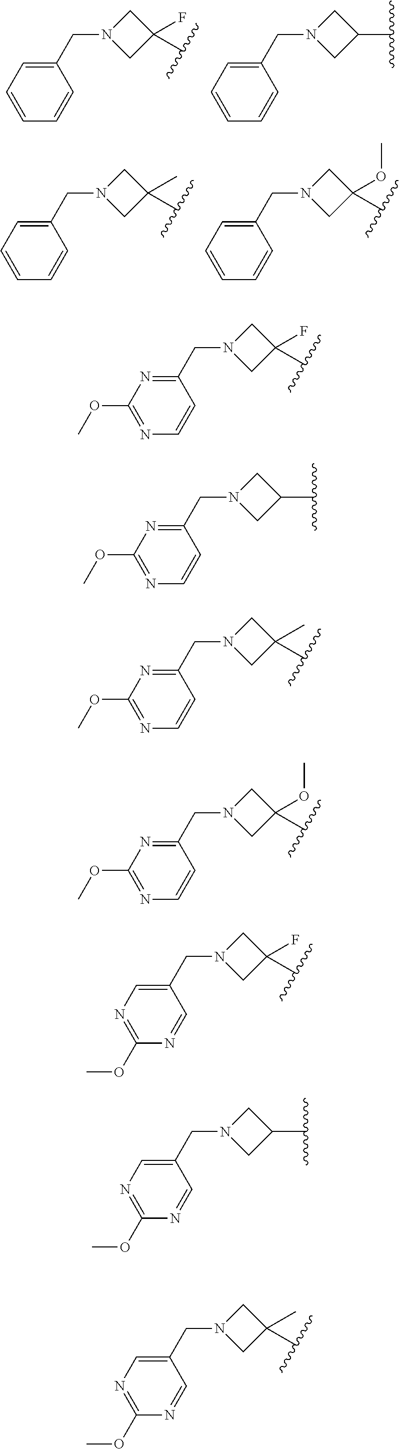

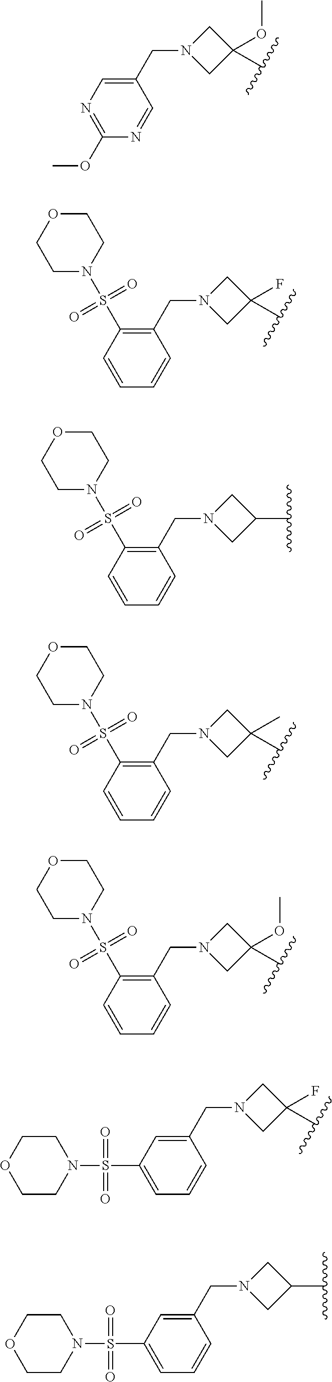

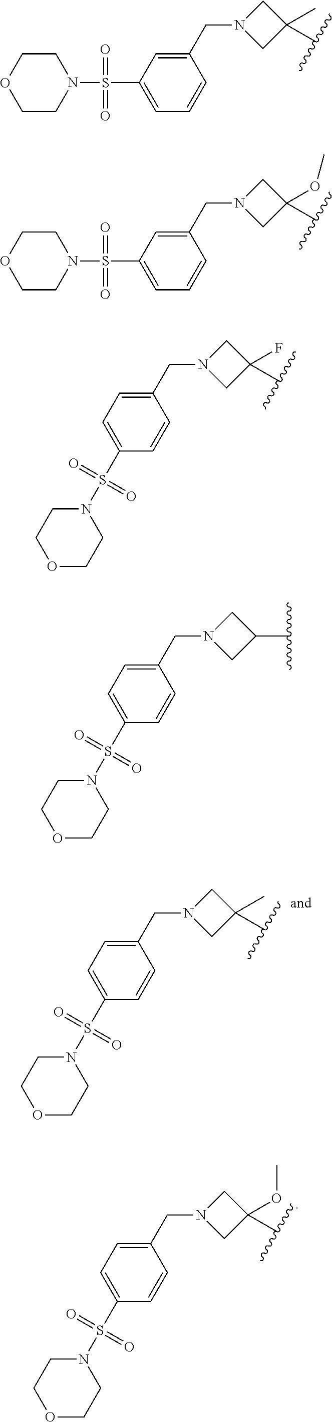 [SMILES: *C1(C)CN(Cc2cccc(S(=O)(=O)N3CCOCC3)c2)C1 ; *C1(OC)CN(Cc2cccc(S(=O)(=O)N3CCOCC3)c2)C1 ; *C1(F)CN(Cc2ccc(S(=O)(=O)N3CCOCC3)cc2)C1 ; *C1CN(Cc2ccc(S(=O)(=O)N3CCOCC3)cc2)C1 ; *C1(C)CN(Cc2ccc(S(=O)(=O)N3CCOCC3)cc2)C1 ; *C1(OC)CN(Cc2ccc(S(=O)(=O)N3CCOCC3)cc2)C1]

In certain embodiments, one R¹, R², or R¹′ is selected from:

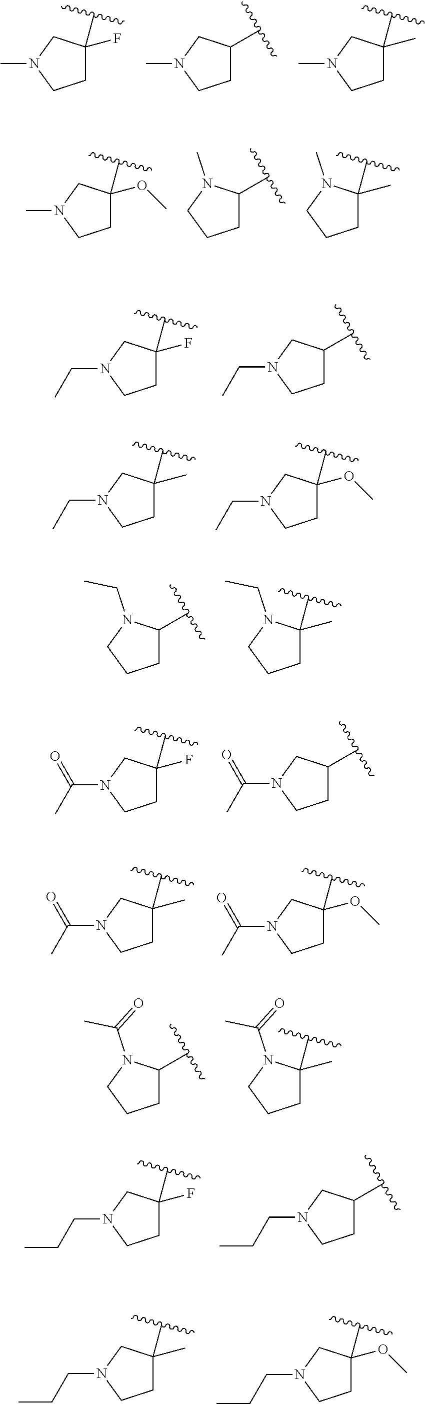

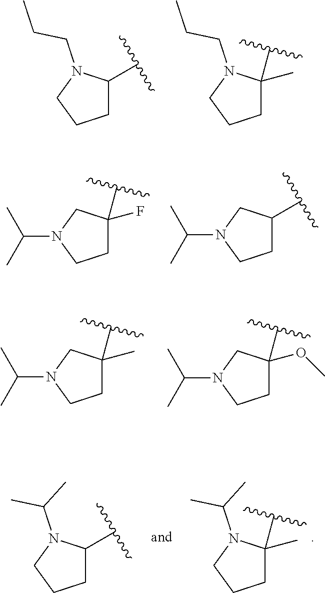

In certain embodiments, one R¹, R², or R¹′ is selected from:

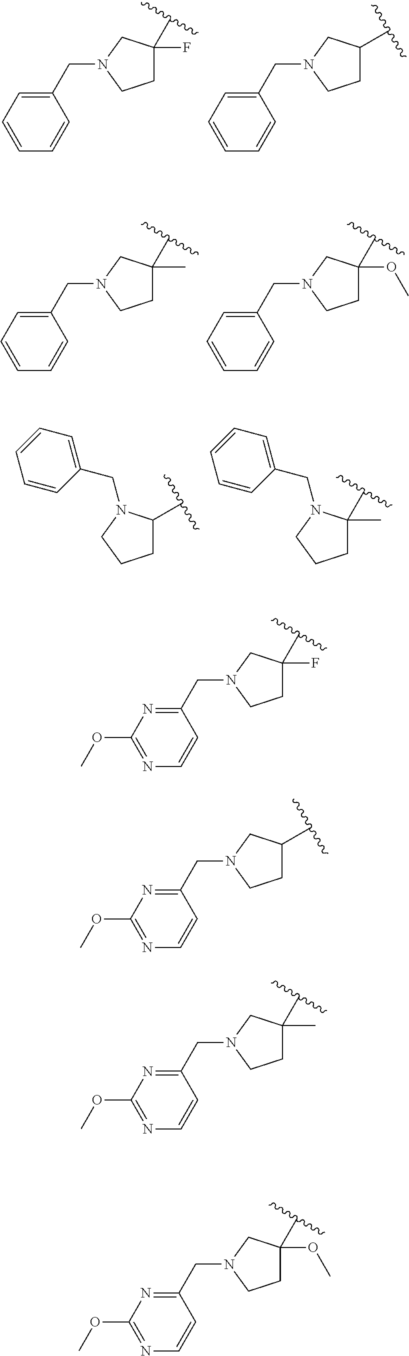

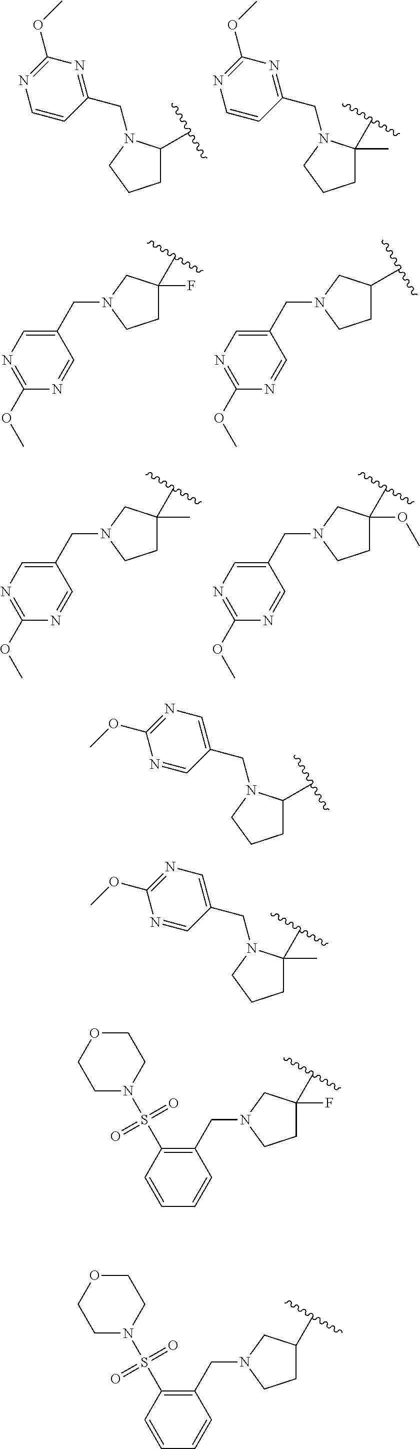

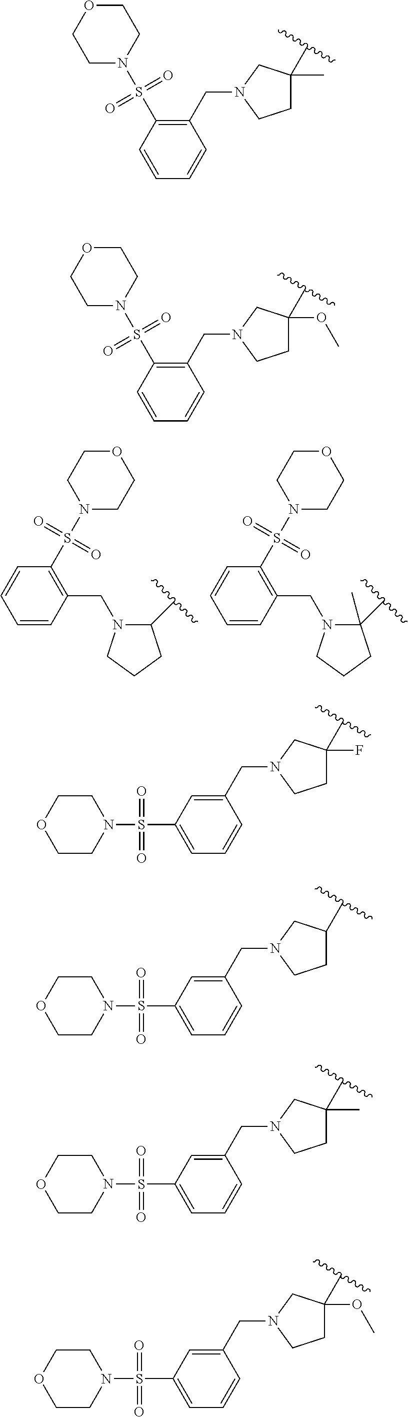

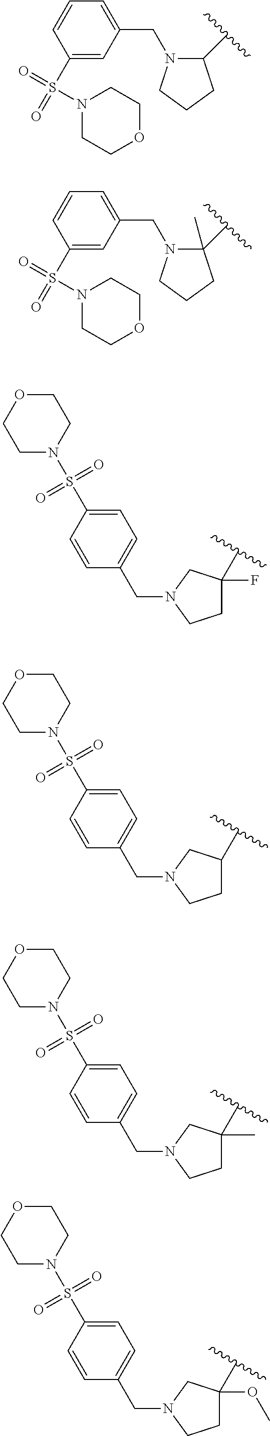

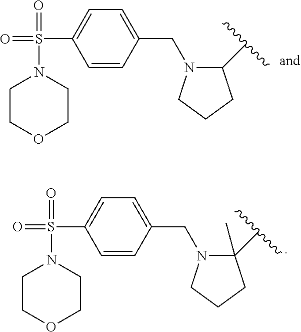 [SMILES: *C1CCCN1Cc1ccc(S(=O)(=O)N2CCOCC2)cc1 ; *C1(C)CCCN1Cc1ccc(S(=O)(=O)N2CCOCC2)cc1]

In certain embodiments, one R¹, R², or R¹′ is selected from:

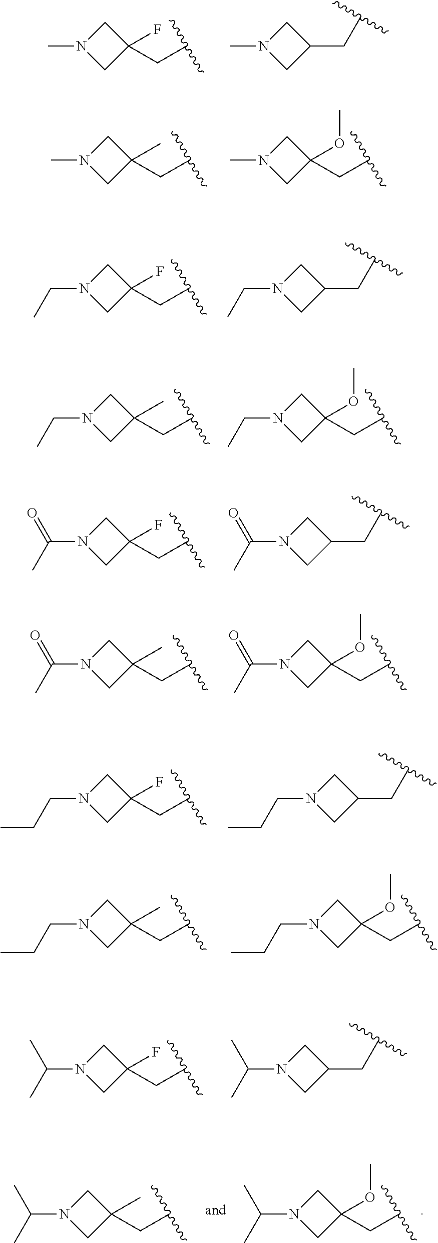

In certain embodiments, one R¹, R², or R¹′ is selected from:

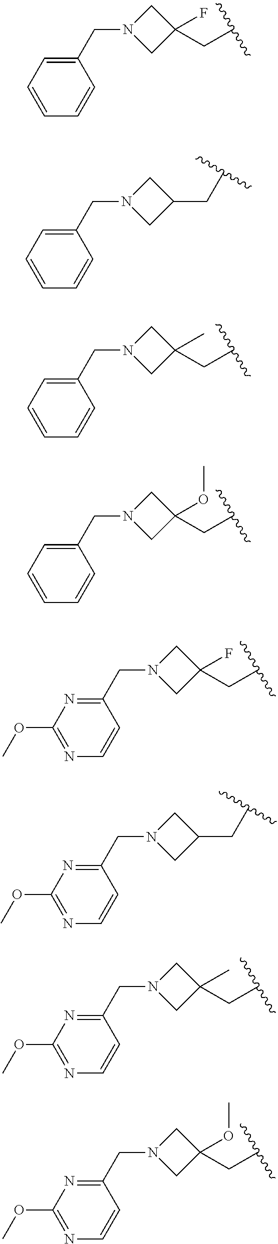

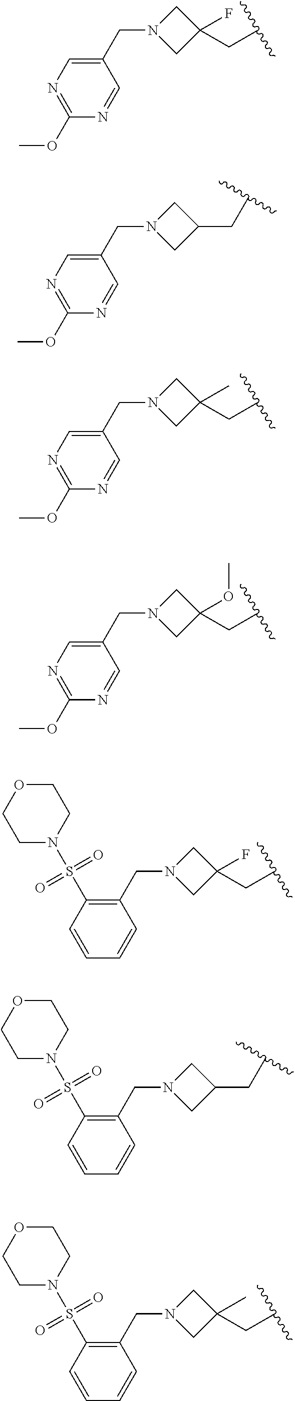

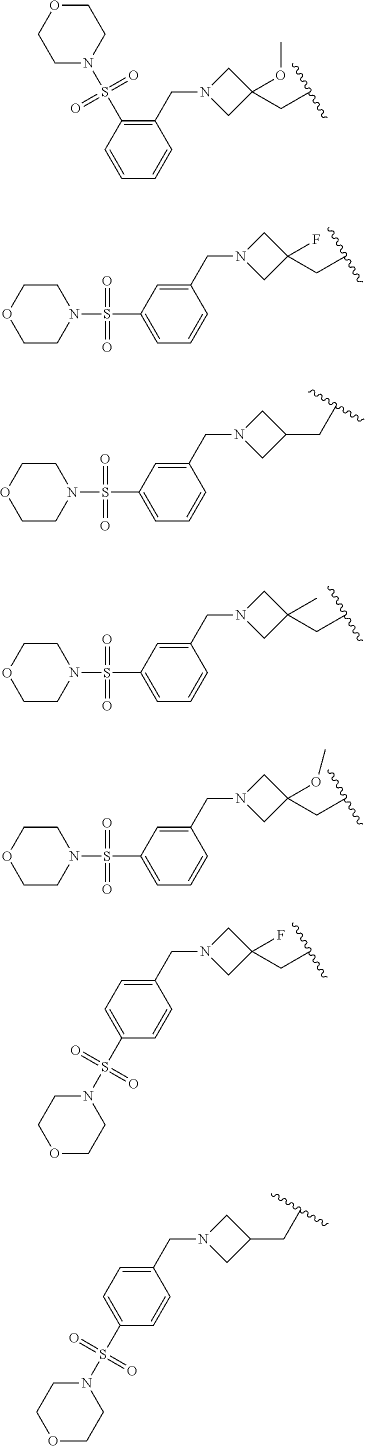

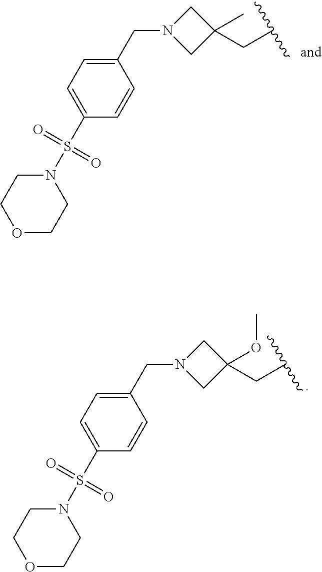 [SMILES: *CC1(C)CN(Cc2ccc(S(=O)(=O)N3CCOCC3)cc2)C1 ; *CC1(OC)CN(Cc2ccc(S(=O)(=O)N3CCOCC3)cc2)C1]

In certain embodiments, one R¹, R², or R¹′ is selected from:

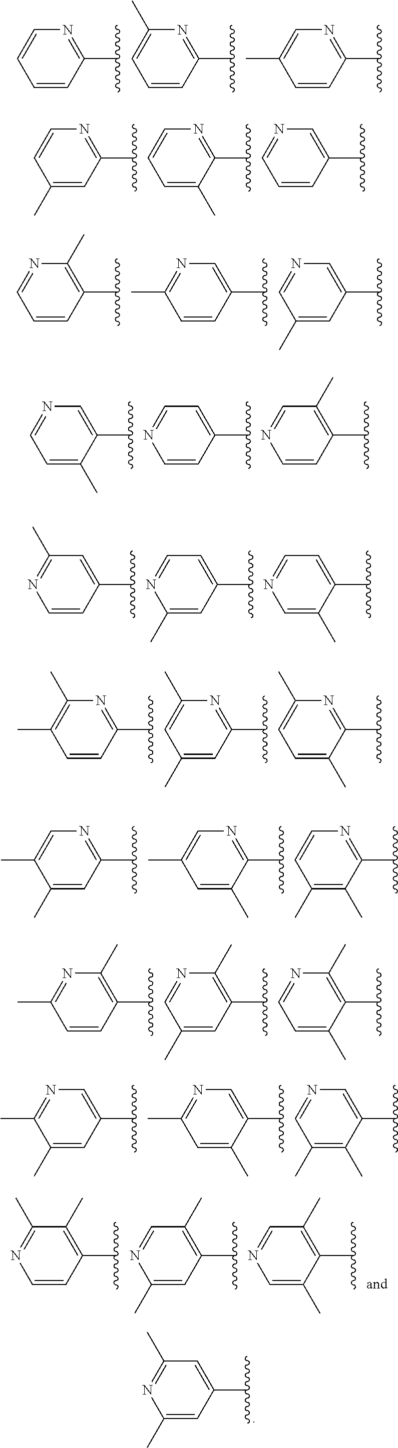

In certain embodiments, one R¹, R², or R¹′ is selected from:

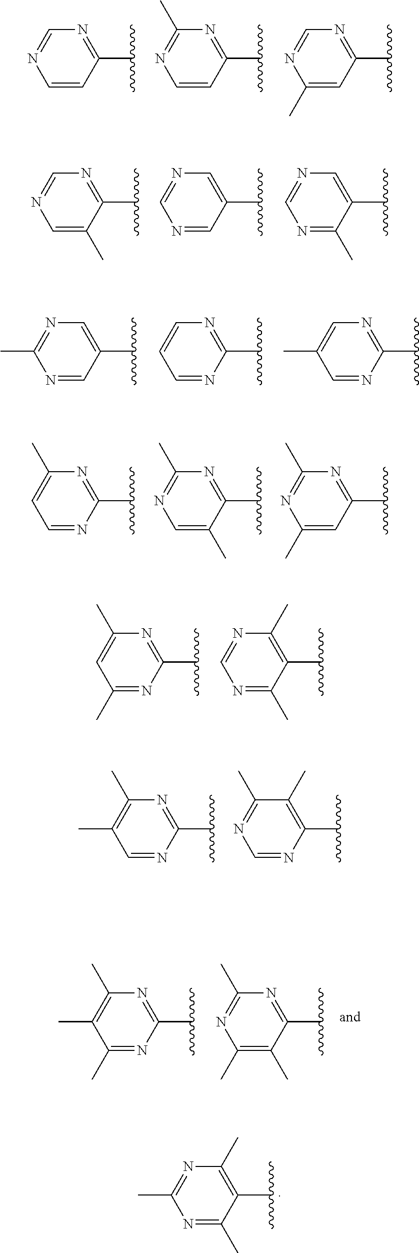

In certain embodiments, one R¹, R², or R¹′ is selected from:

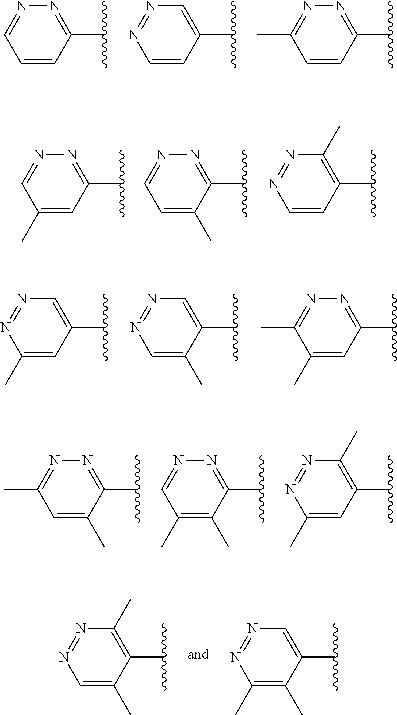

In certain embodiments, one R¹, R², or R¹′ is selected from:

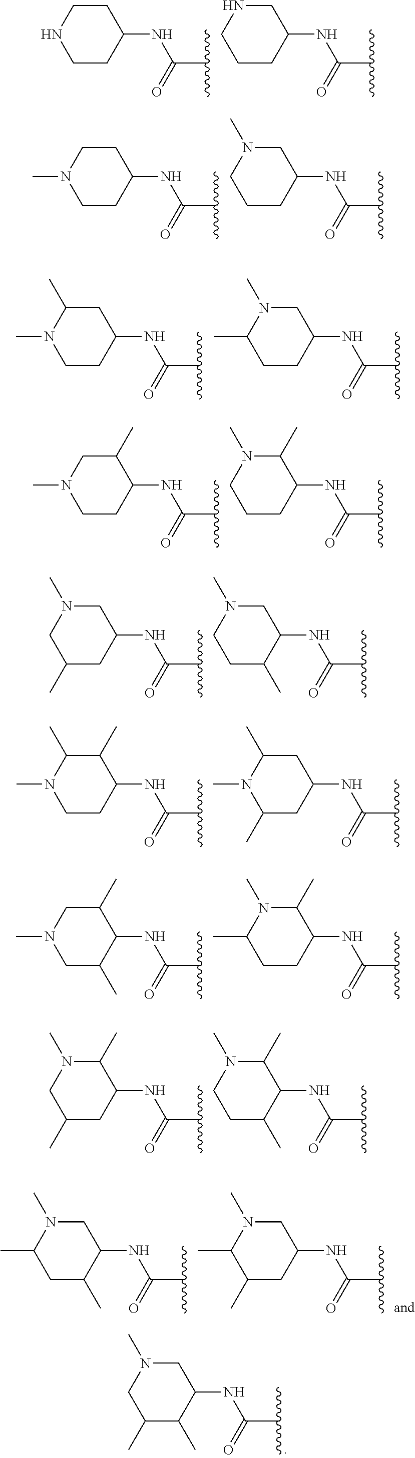

In certain embodiments, one R¹, R², or R¹′ is selected from:

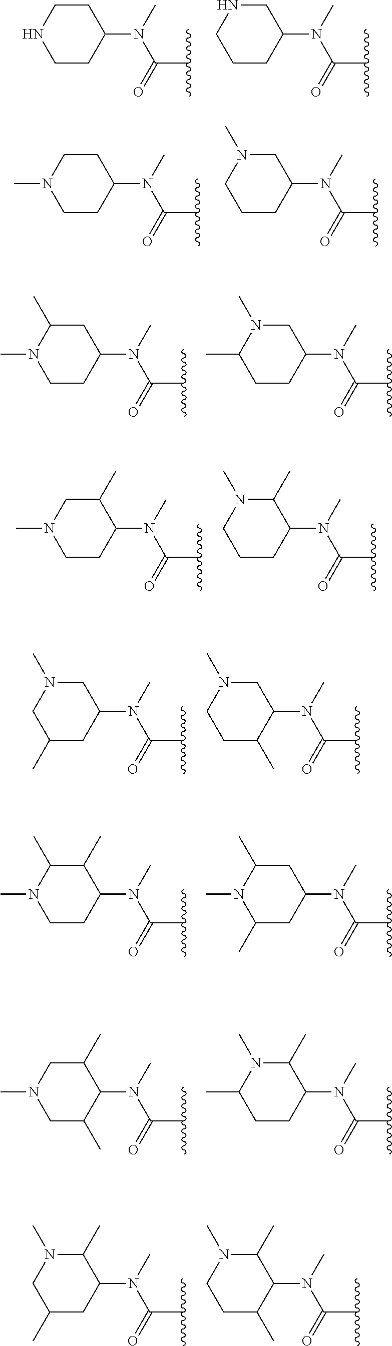

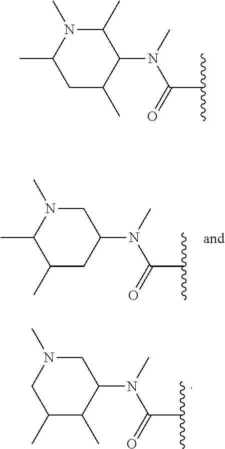

In certain embodiments, one R¹, R², or R¹′ is selected from:

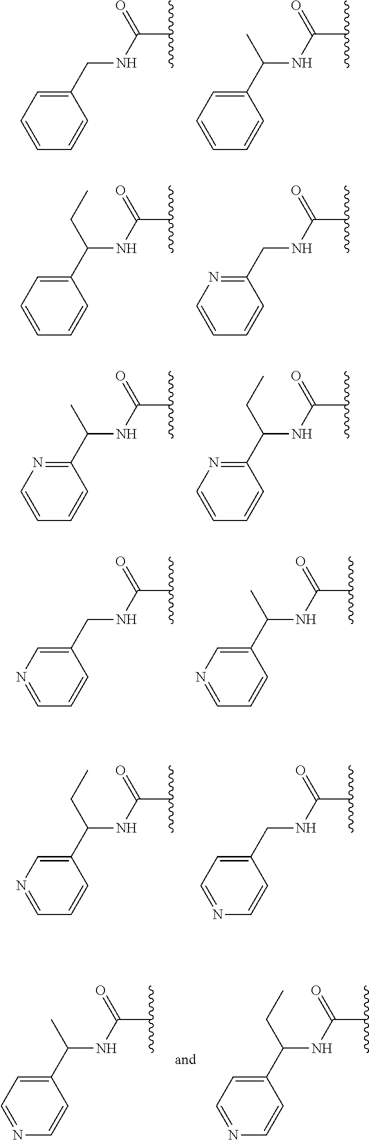

In certain embodiments, one R¹, R², or R¹′ is selected from:

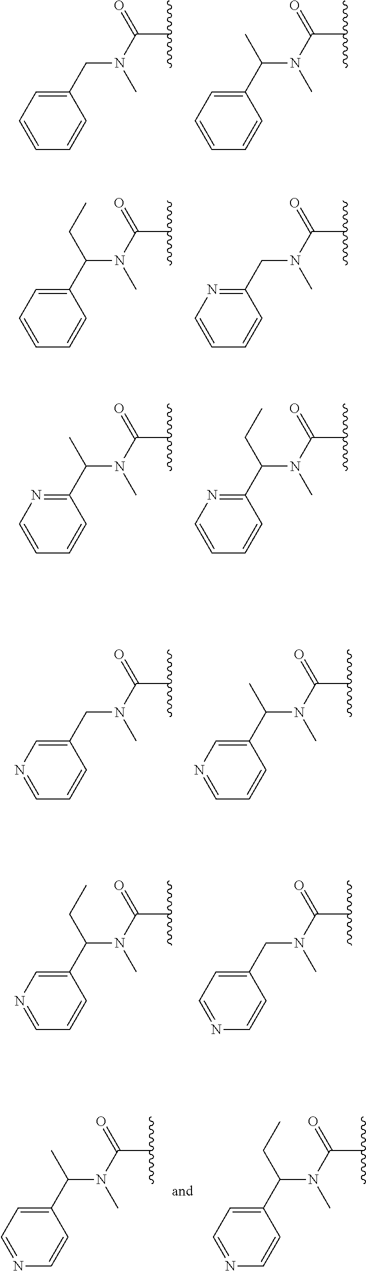

In certain embodiments, one R¹, R², or R¹′ is selected from:

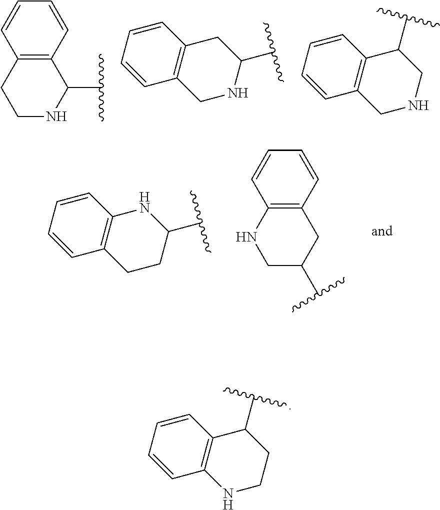

In certain embodiments, one R¹, R², or R¹′ is selected from:

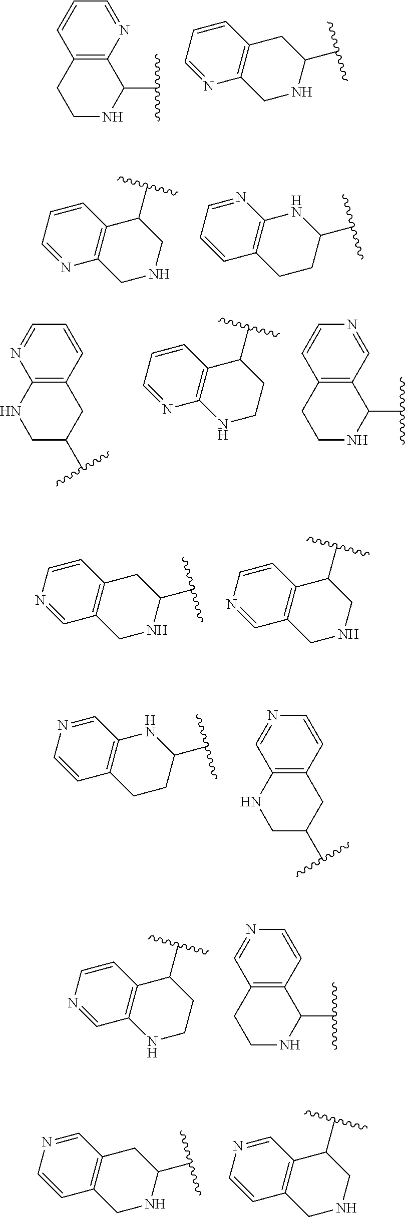

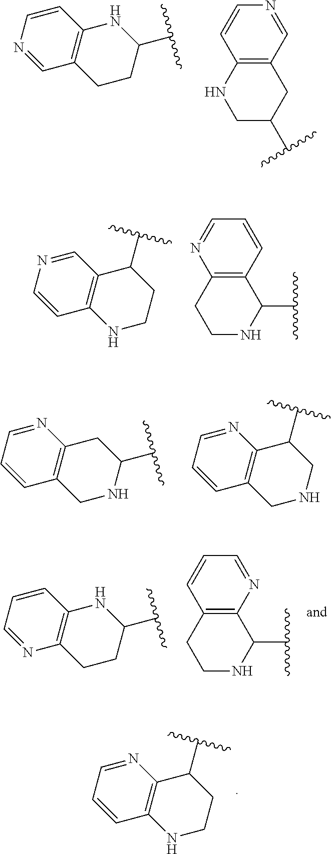

In certain embodiments, one R¹, R², or R¹′ is selected from:

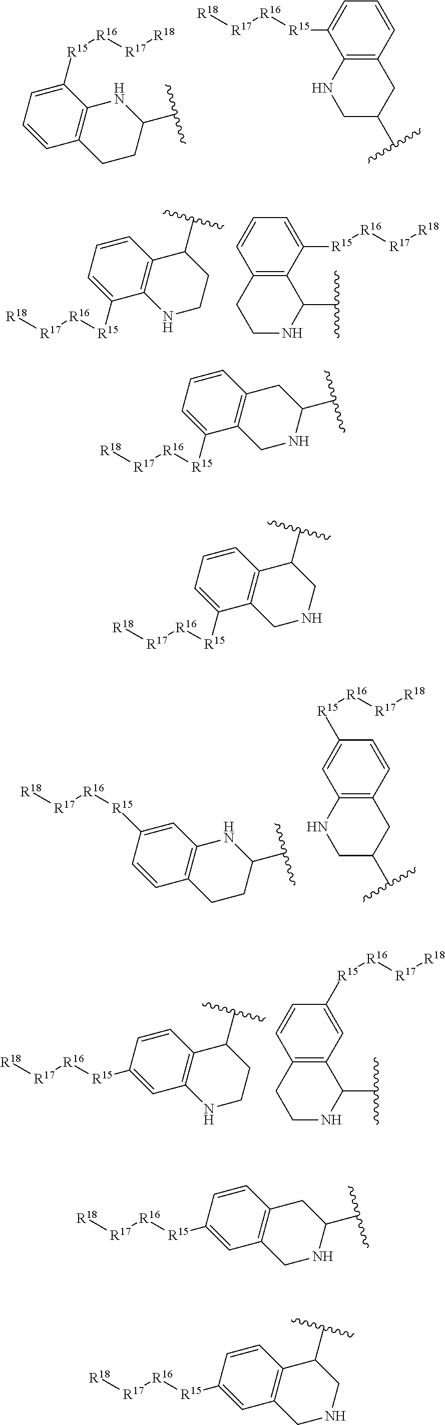

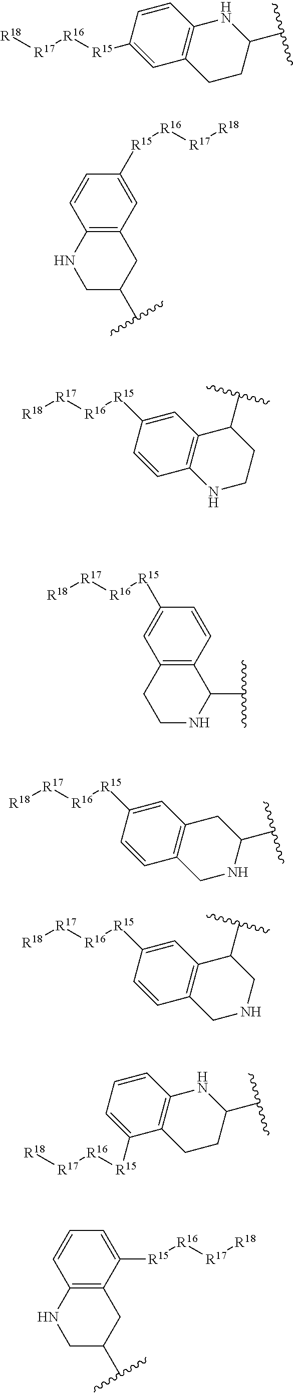

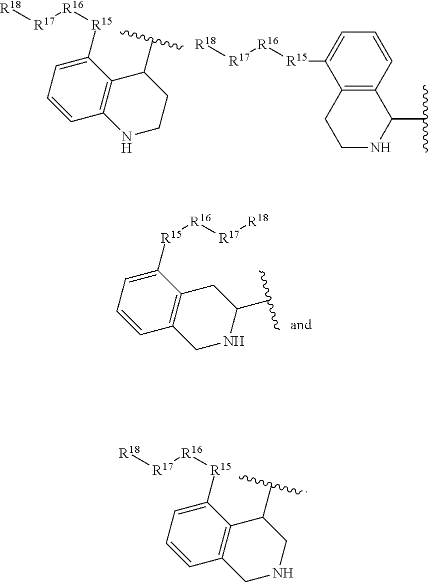

In certain embodiments, one R¹, R², or R¹′ is selected from:

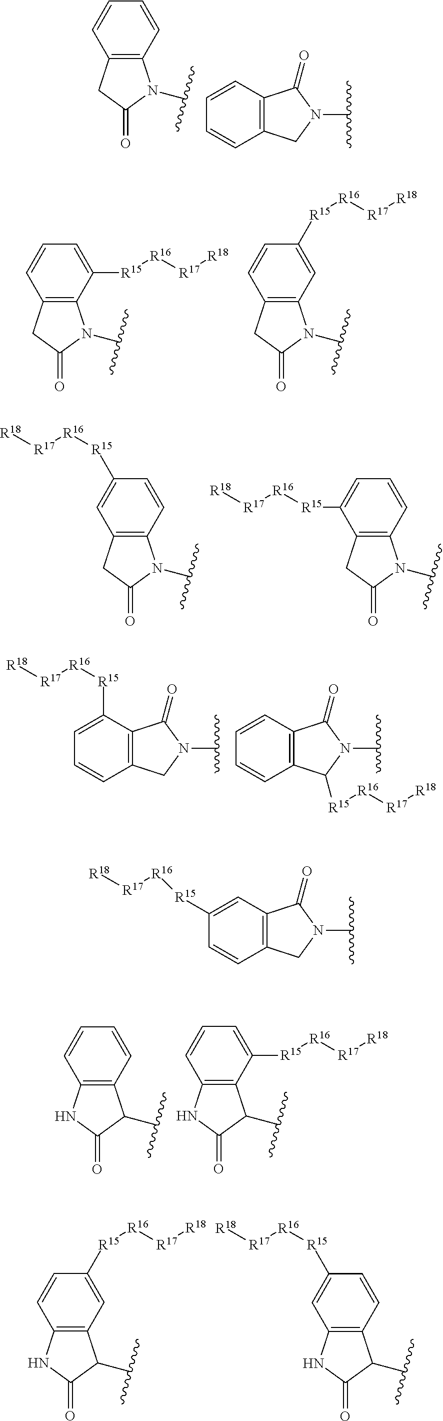

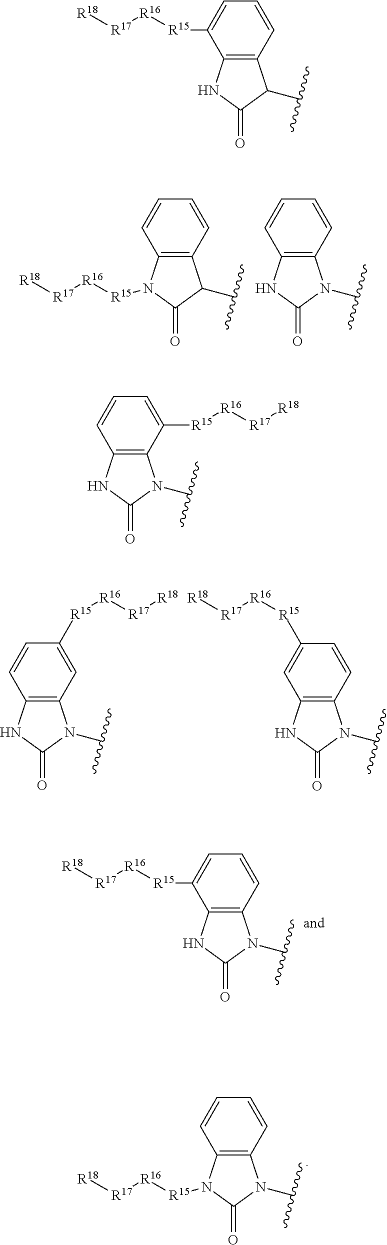

In certain embodiments, one R¹, R², or R¹′ is selected from:

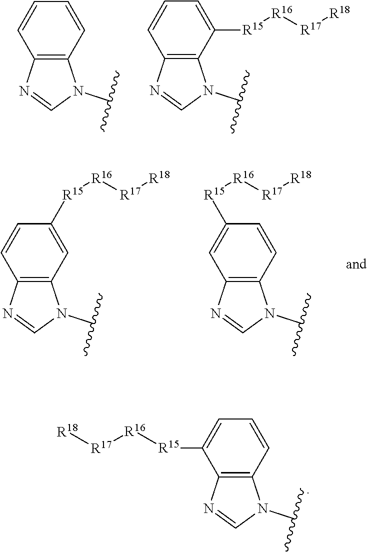

In certain embodiments, one R¹, R², or R¹′ is selected from:

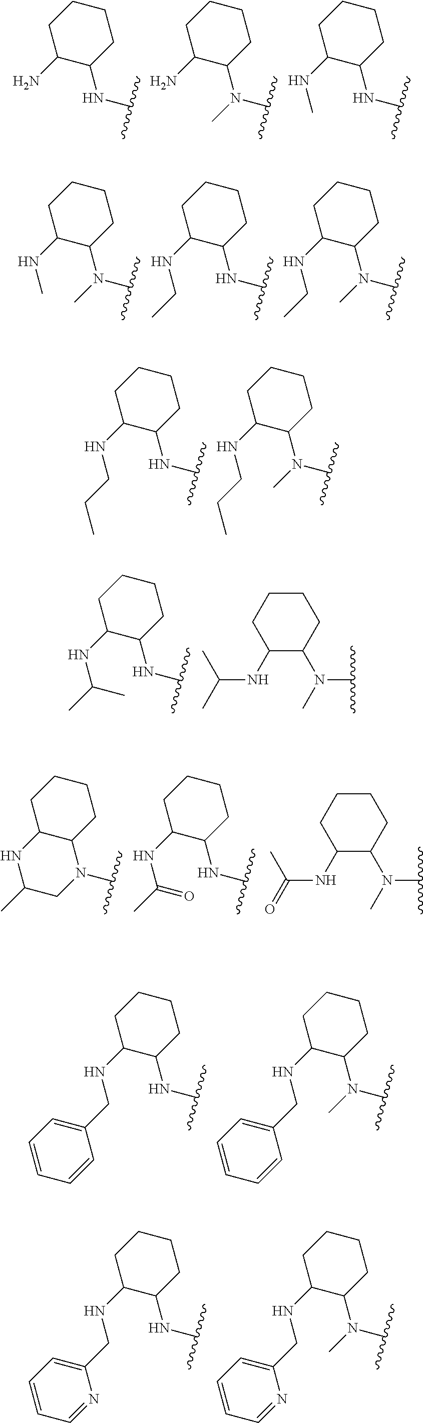

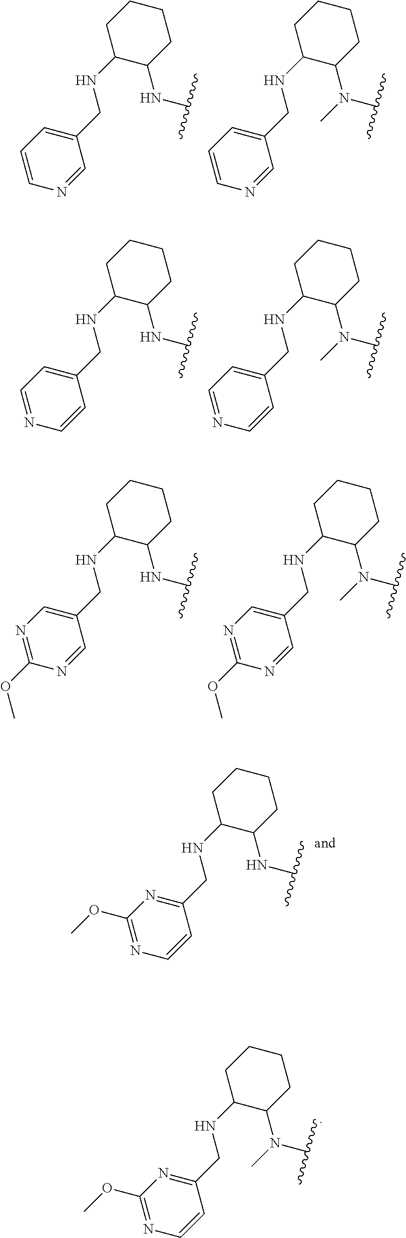

In certain embodiments, one R¹, R², or R¹′ is selected from:

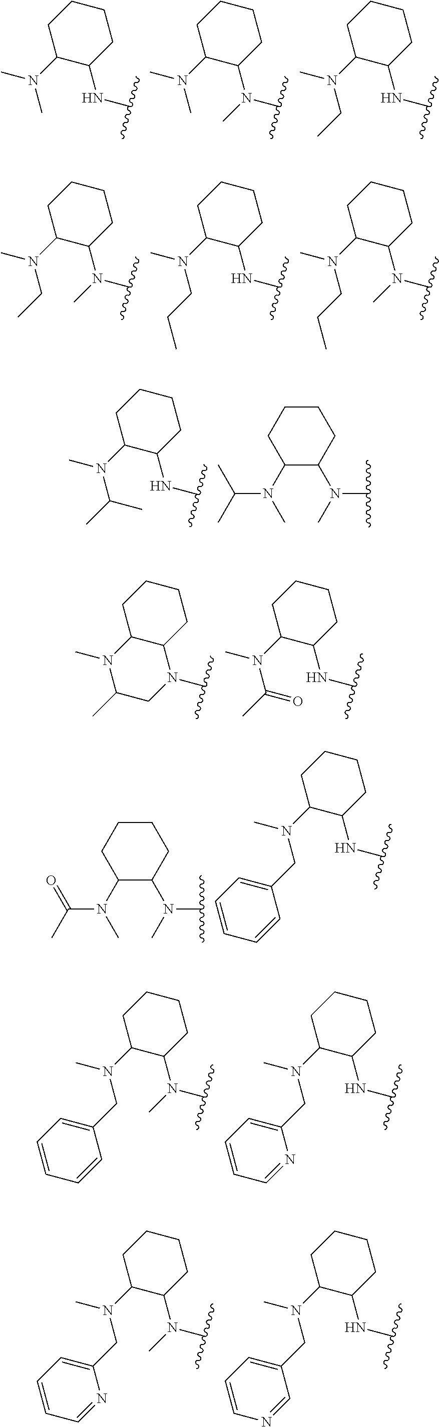

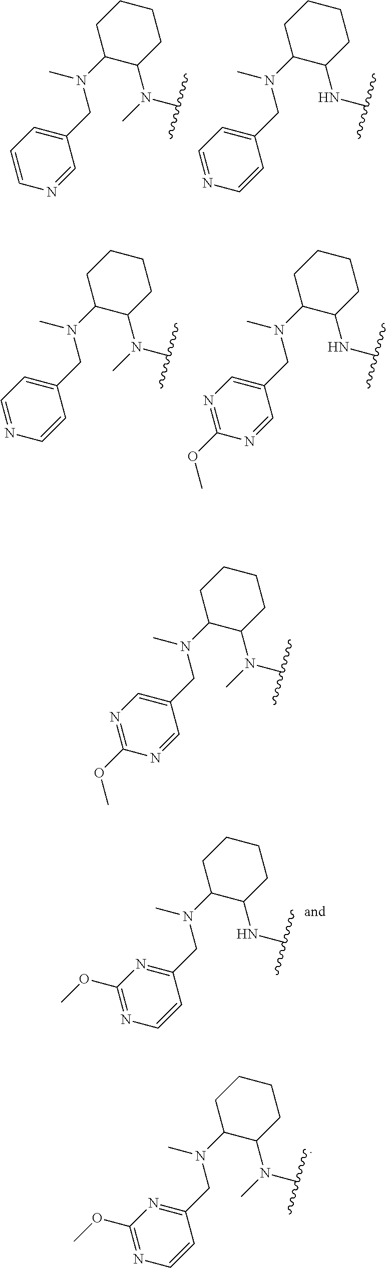

In certain embodiments, one R¹, R², or R¹′ is selected from

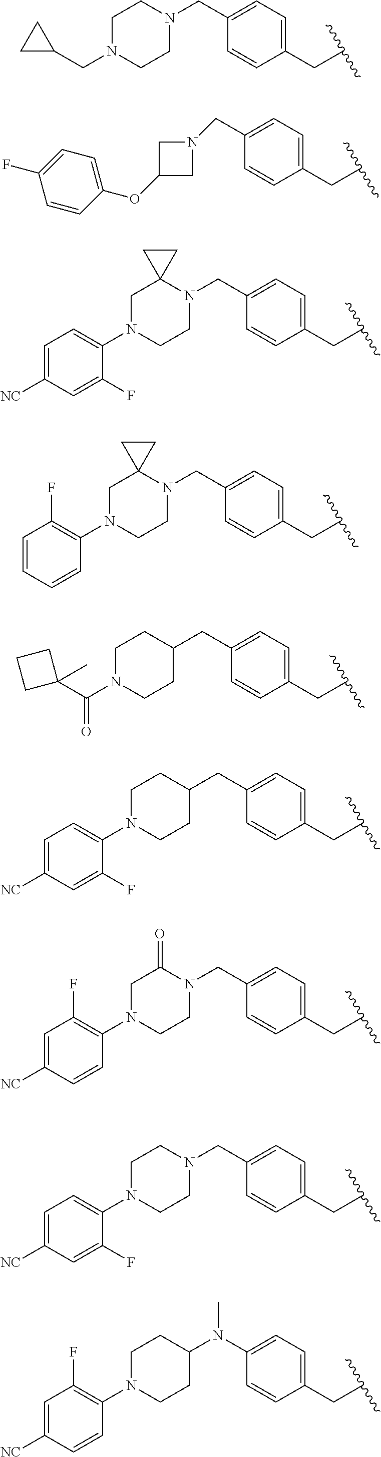

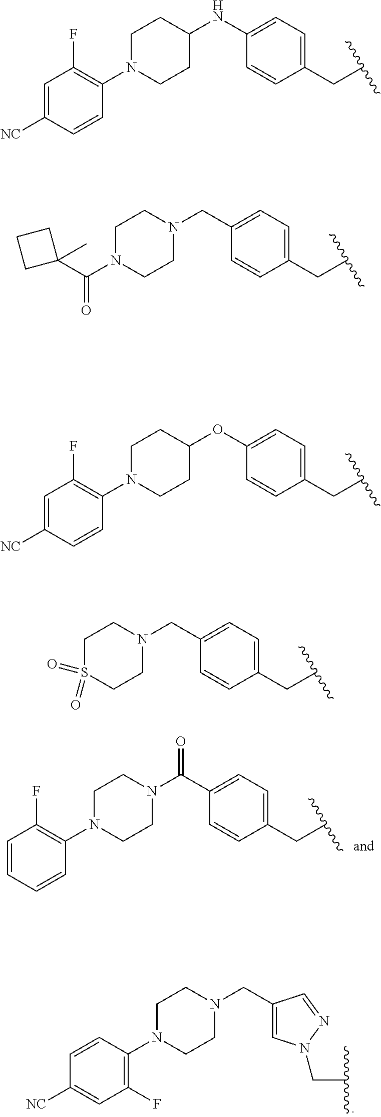 [SMILES: *Cc1ccc(NC2CCN(c3ccc(C#N)cc3F)CC2)cc1 ; *Cc1ccc(CN2CCN(C(=O)C3(C)CCC3)CC2)cc1 ; *Cc1ccc(OC2CCN(c3ccc(C#N)cc3F)CC2)cc1 ; *Cc1ccc(CN2CCS(=O)(=O)CC2)cc1 ; *Cc1ccc(C(=O)N2CCN(c3ccccc3F)CC2)cc1 ; *Cn1cc(CN2CCN(c3ccc(C#N)cc3F)CC2)cn1]

### Non-Limiting Embodiments of R³

In certain embodiments R³ is selected from hydrogen and halogen.

In certain embodiments R³ is selected from alkyl and haloalkyl.

In certain embodiments R³ is hydrogen.

In certain embodiments R³ is halogen.

In certain embodiments R³ is alkyl.

In certain embodiments R³ is haloalkyl.

In certain embodiments R³ is fluoro.

In certain embodiments R³ is chloro.

In certain embodiments R³ is bromo.

In certain embodiments R³ is iodo.

In certain embodiments R³ is methyl.

In certain embodiments R³ is ethyl.

In certain embodiments R³ is trifluoromethyl.

In certain embodiments R³ is pentafluoroethyl.

In certain embodiments R³ is difluoromethyl.

In certain embodiments R³ is fluoromethyl.

In certain embodiments R³ is combined with an R⁴ group to form a 1 carbon attachment.

In certain embodiments R³ is combined with an R⁴ group to form a 2 carbon attachment.

In certain embodiments R³ is combined with an R⁴ group to form a 3 carbon attachment.

In certain embodiments R³ is combined with an R⁴ group to form a 4 carbon attachment.

In certain embodiments R³ is combined with an R⁴ group to form a double bond.

### Non-Limiting Embodiments of R⁶ and R⁷

In certain embodiments R⁶ and R⁷ are independently selected from hydrogen, alkyl, halogen, and haloalkyl.

In certain embodiments R⁶ and R⁷ are independently selected from —OR¹⁰, —SR¹⁰, —S(O)R¹², —SO₂R¹², and —NR¹⁰R¹¹.

In certain embodiments R⁶ and R⁷ are independently selected from alkyl, —OR¹⁰, —SR¹⁰, and —NR¹⁰R¹¹.

In certain embodiments R⁶ is combined with an R³ group to form a 1 carbon attachment.

In certain embodiments R⁶ is combined with an R³ group to form a 2 carbon attachment.

### Embodiments of Cycle-A, Cycle-B, Cycle-C, Cycle-D, Cycle-E, and Cycle-F

In certain embodiments

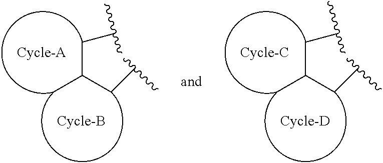

are selected from the following:

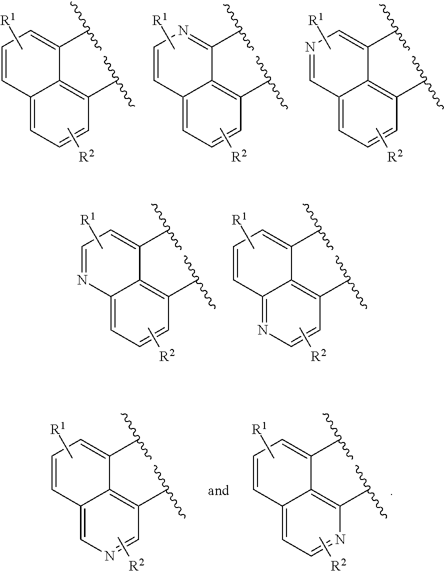

In certain embodiments

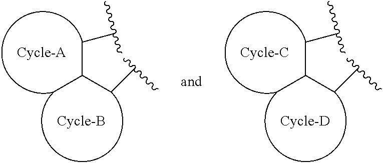

are selected from the following:

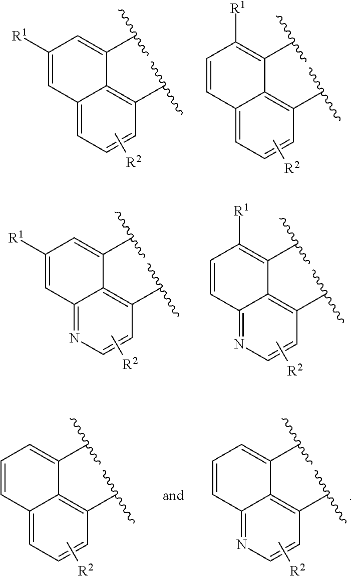

In certain embodiments

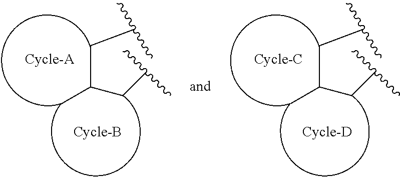

are selected from:

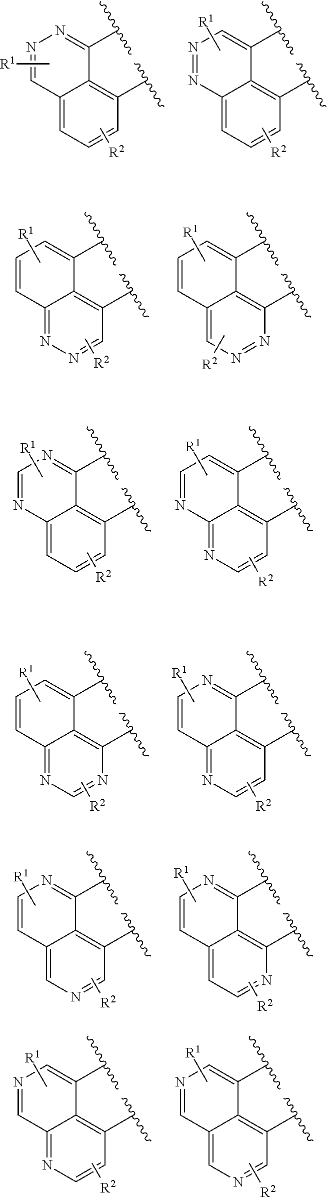

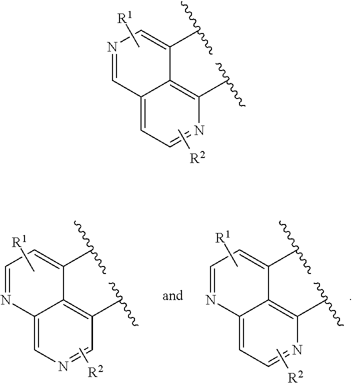

In certain embodiments

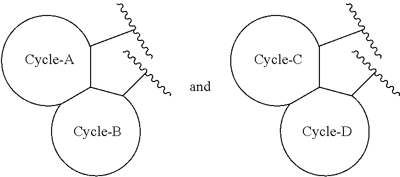

are selected from:

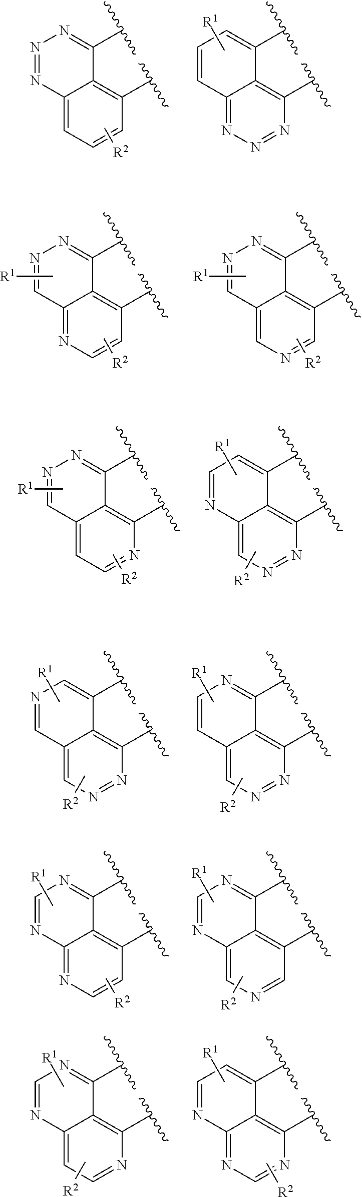

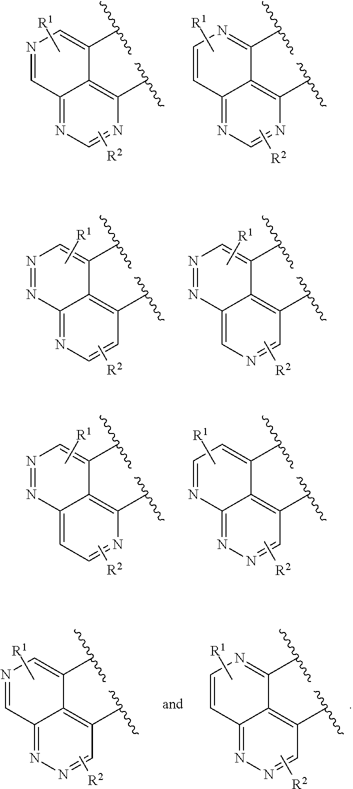

In certain embodiments

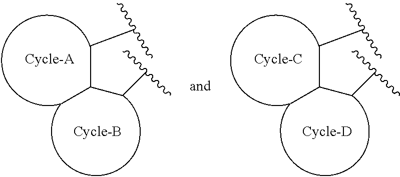

are selected from:

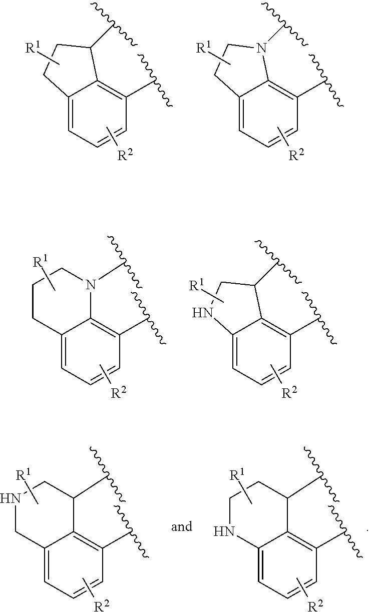

In certain embodiments

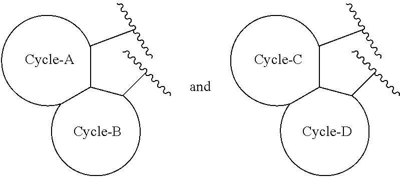

are selected from:

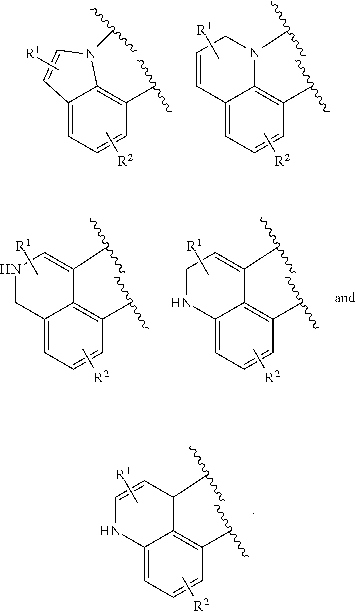

In certain embodiments

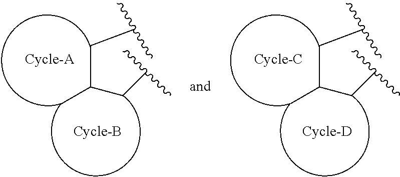

are selected from:

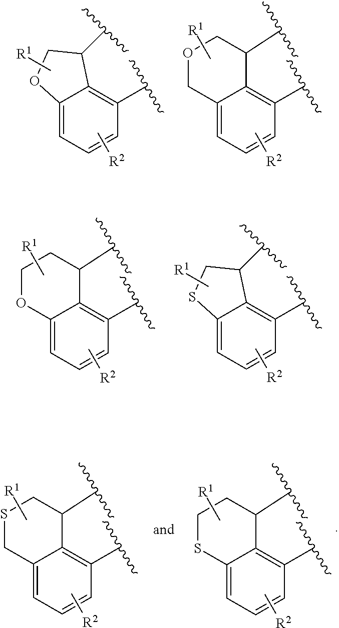

In certain embodiments

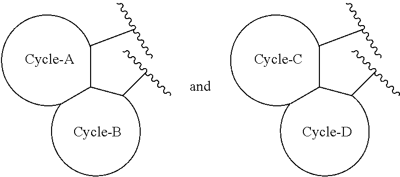

are selected from:

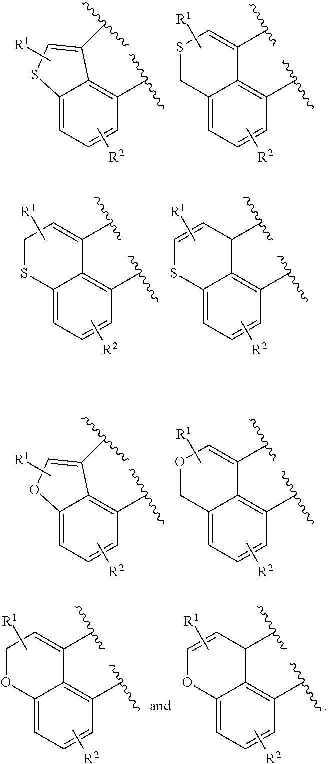

In certain embodiments

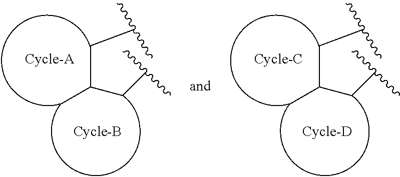

are selected from:

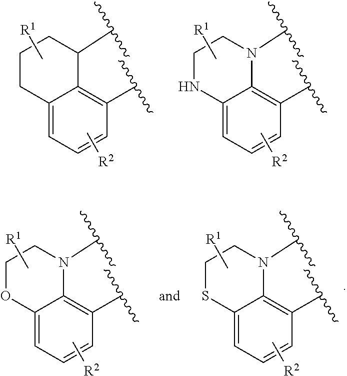

In certain embodiments

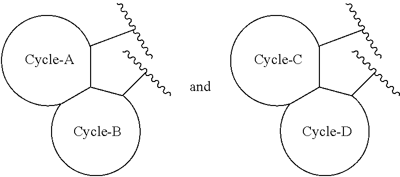

are selected from:

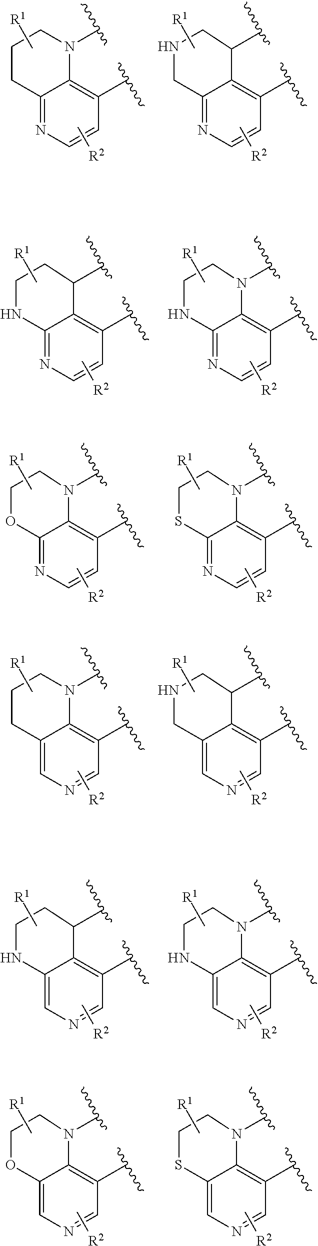

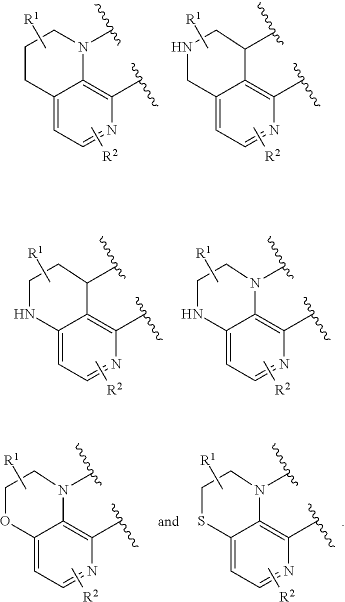

In certain embodiments

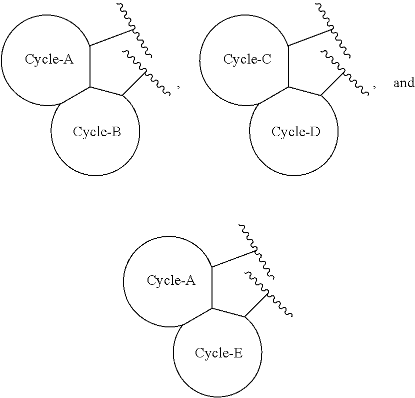

are selected from:

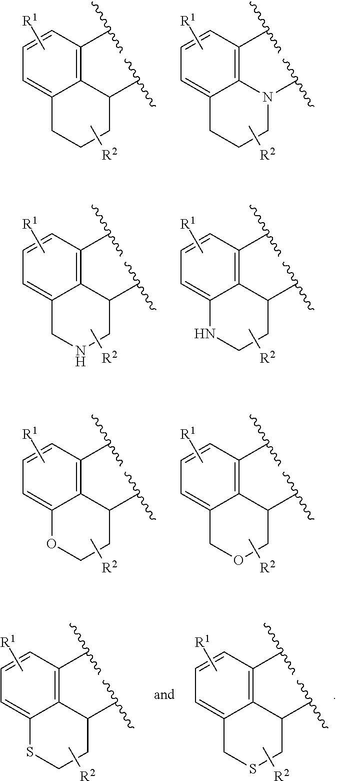

In certain embodiments

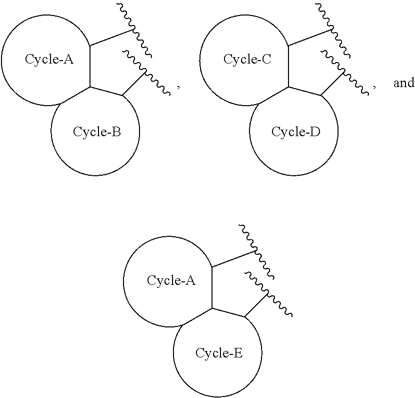

are selected from:

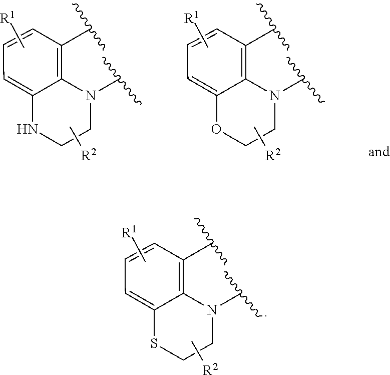

In certain embodiments

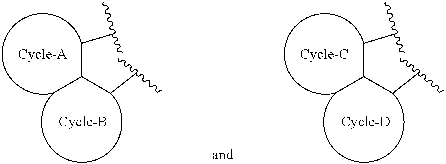

are selected from:

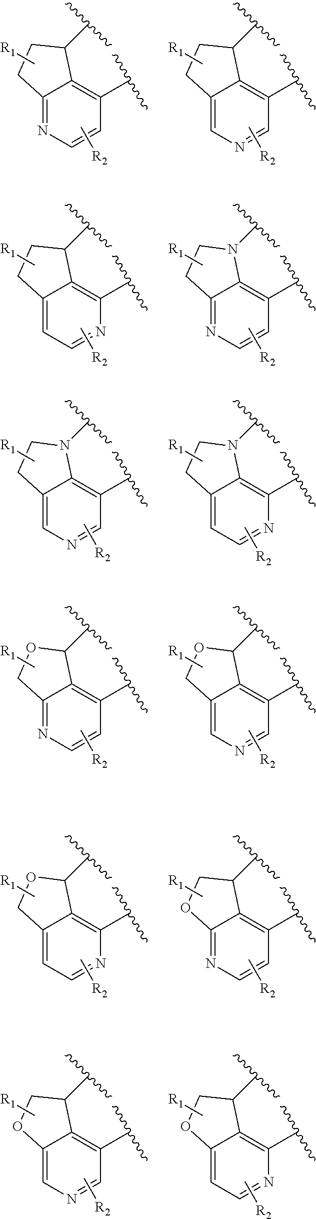

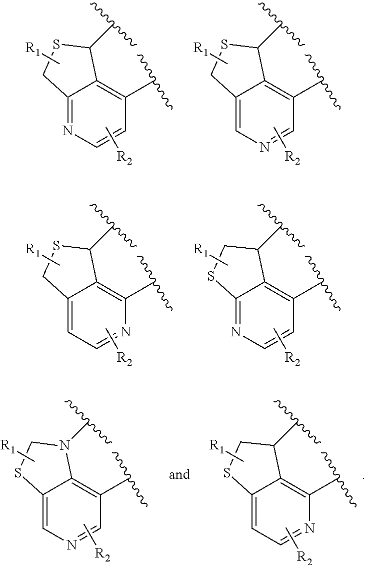

In certain embodiments

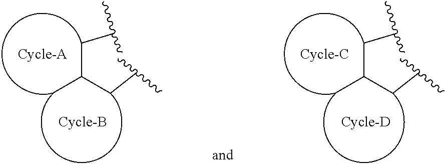

are selected from:

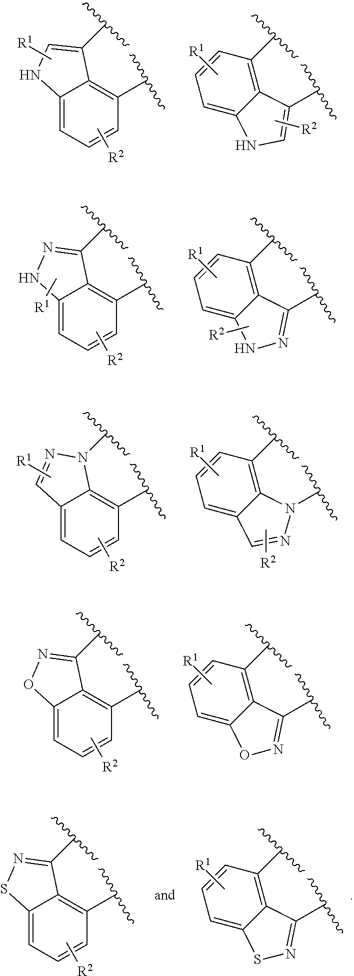

In certain embodiments

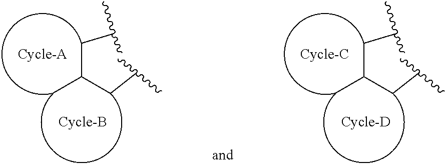

are selected from:

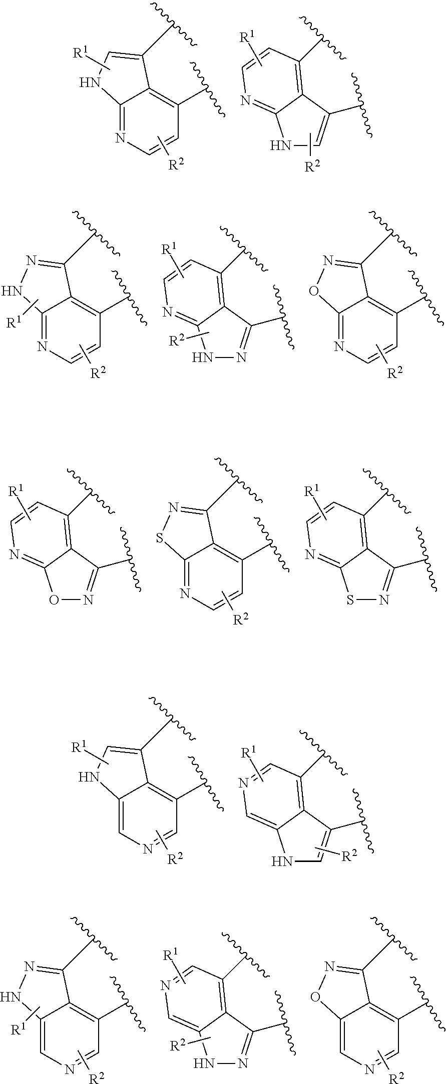

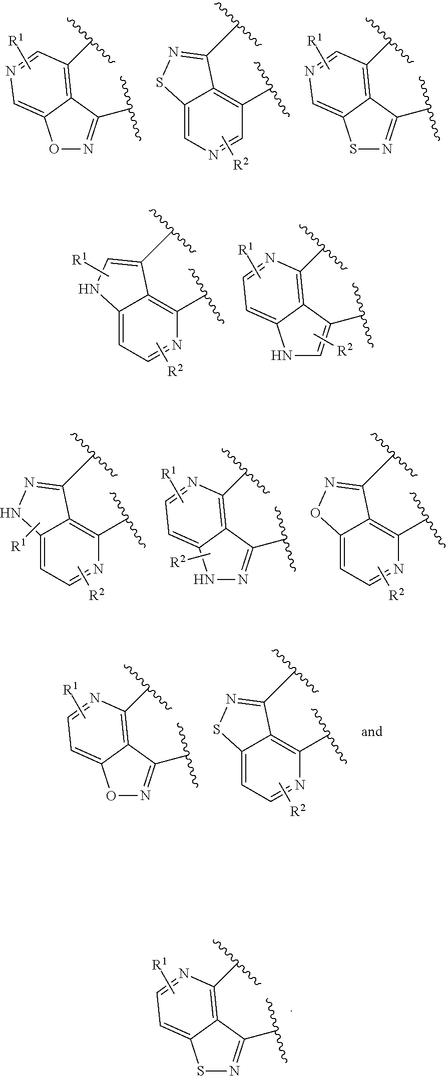

In certain embodiments

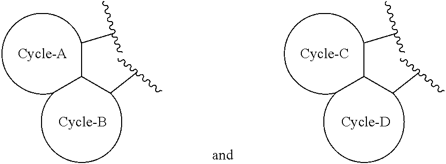

are selected from:

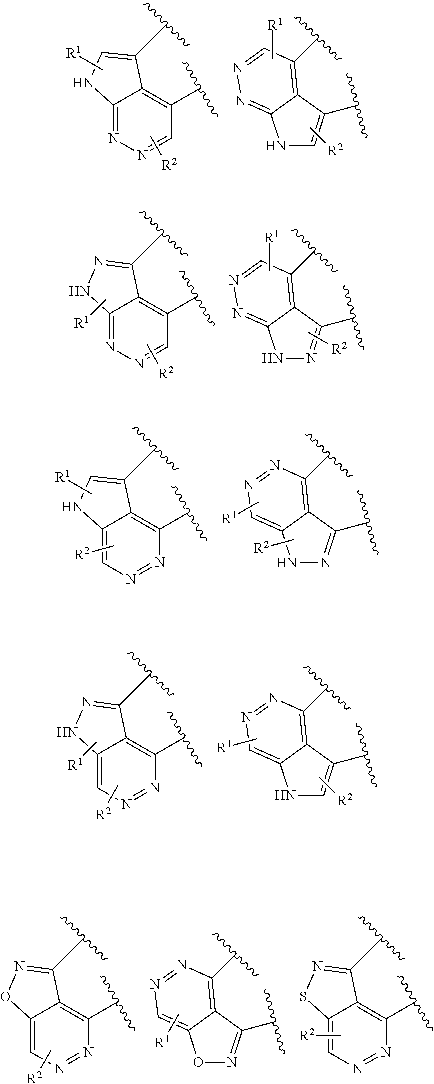

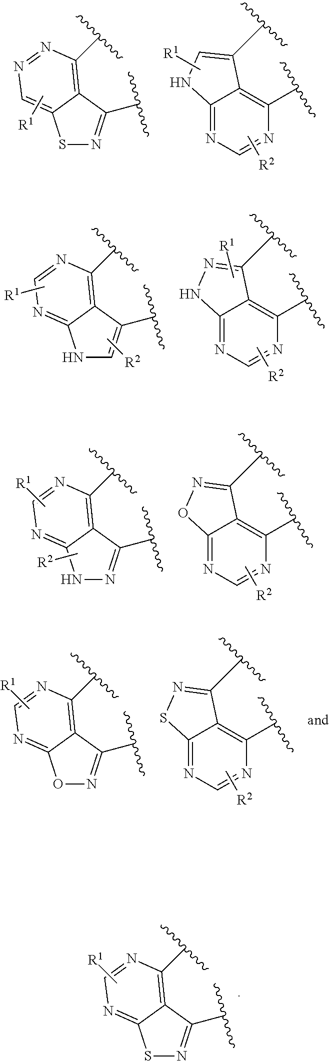

In certain embodiments

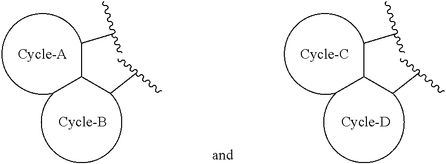

are selected from:

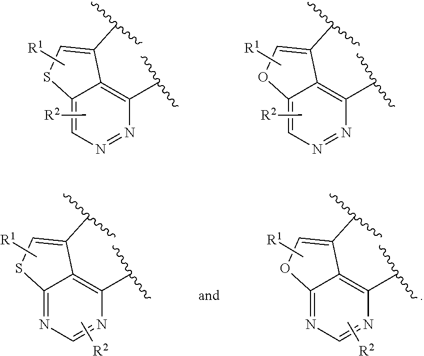

In certain embodiments

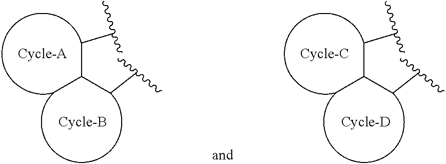

are selected from:

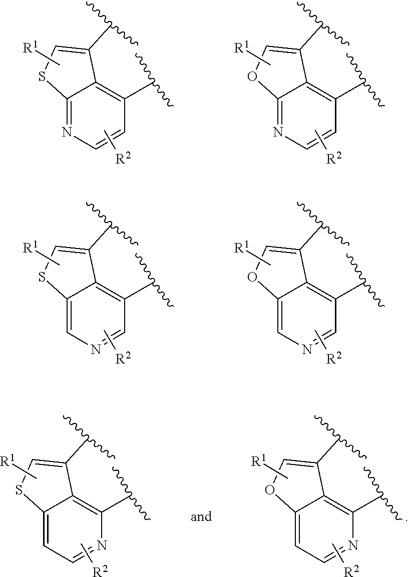

In certain embodiments

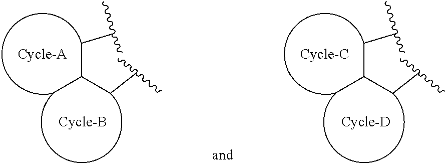

are selected from:

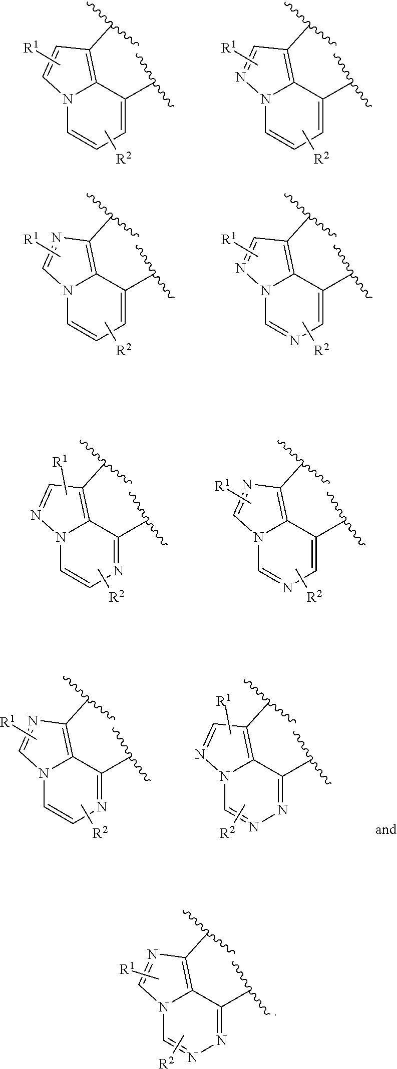

In certain embodiments

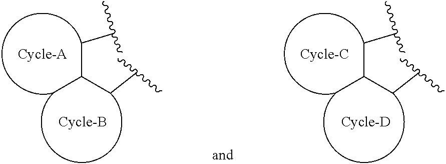

are selected from:

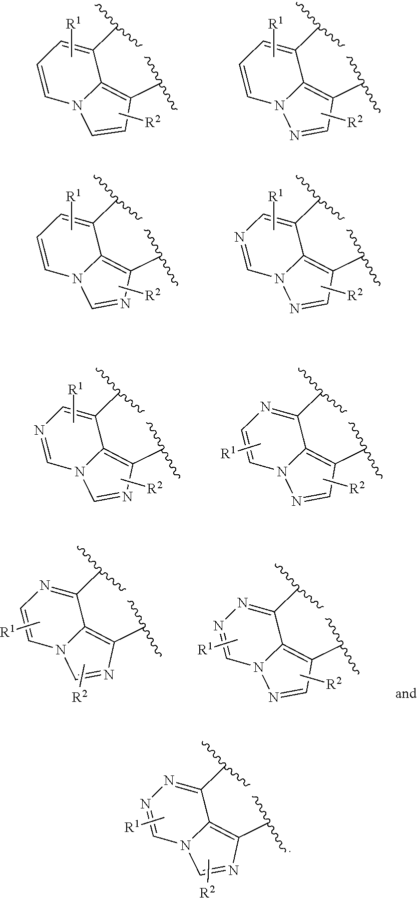

In certain embodiments

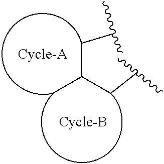 [SMILES: *CC(C)C*]

is selected from:

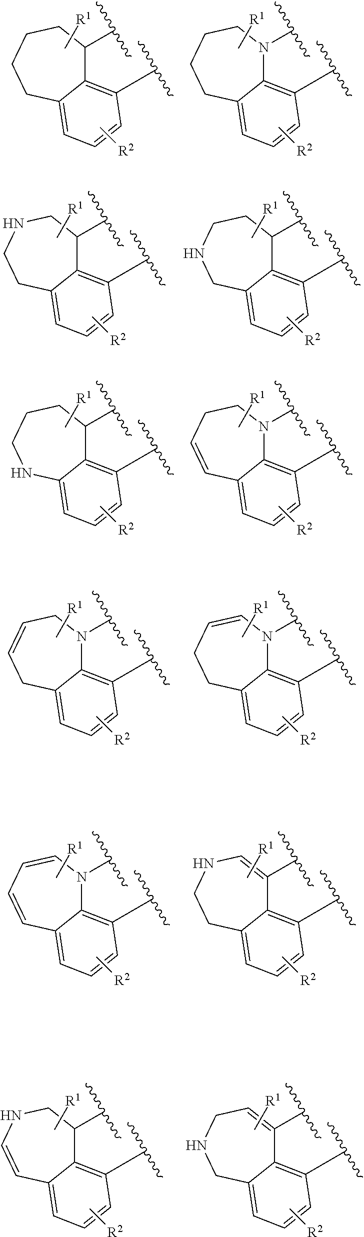

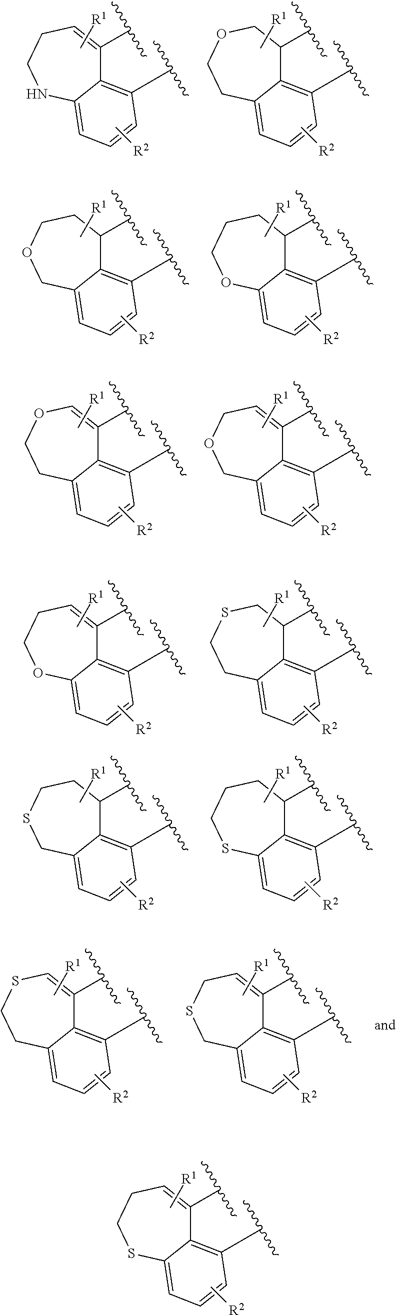

In certain embodiments

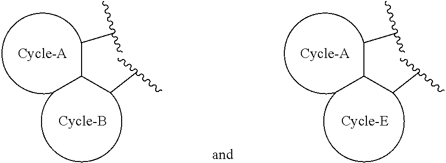

are selected from:

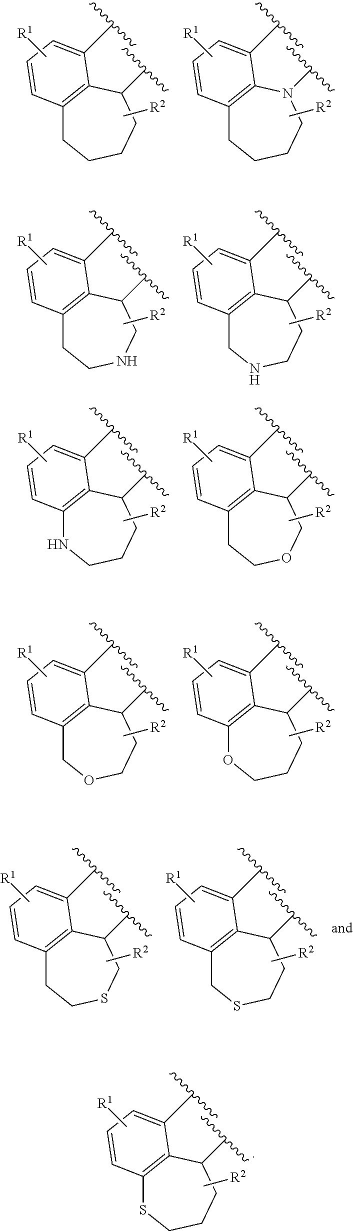

In certain embodiments

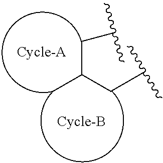 [SMILES: *CC(C)C*]

is selected from:

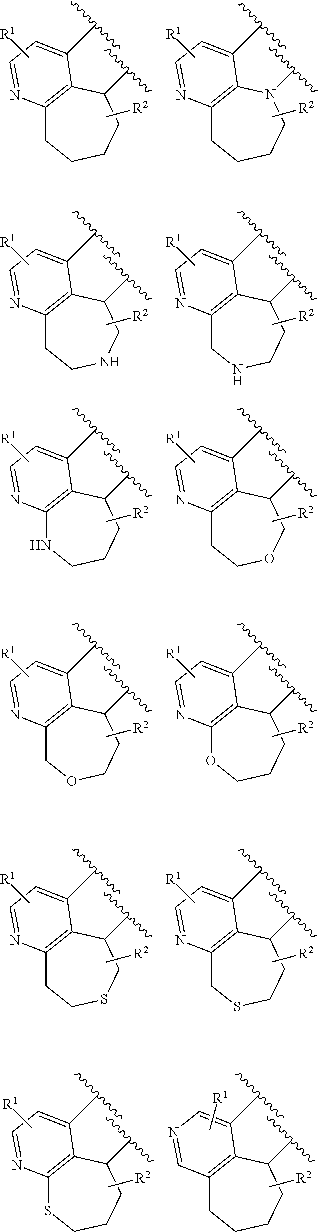

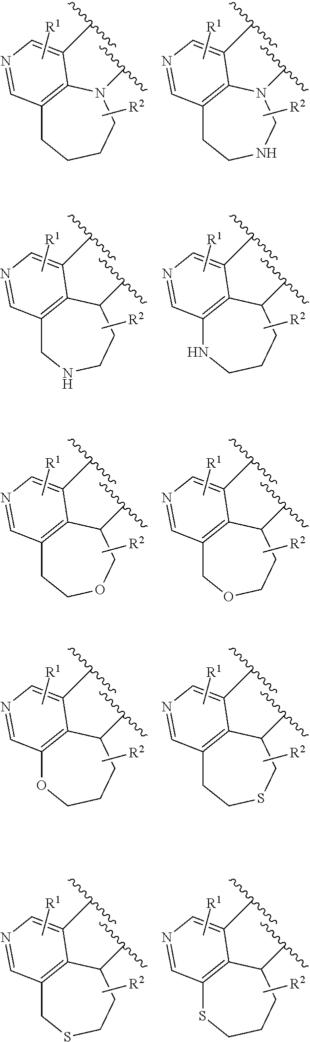

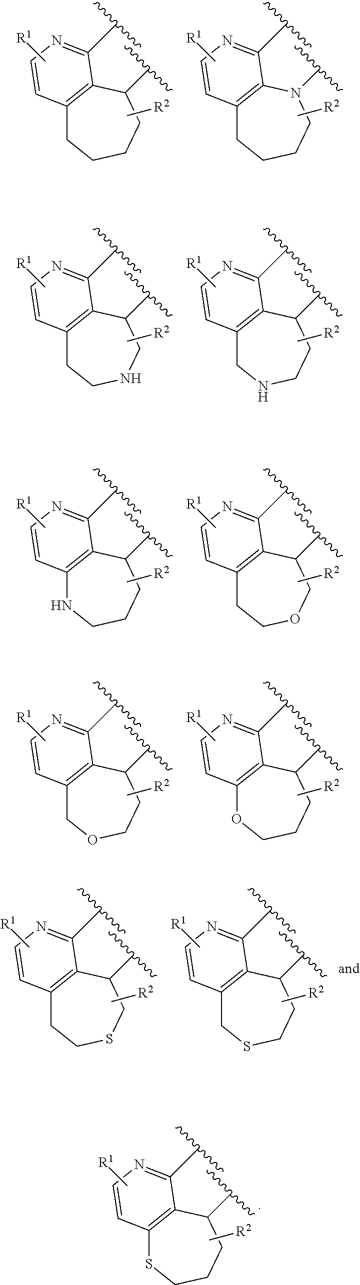

In certain embodiments

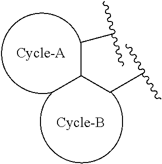 [SMILES: *CC(C)C*]

is selected from:

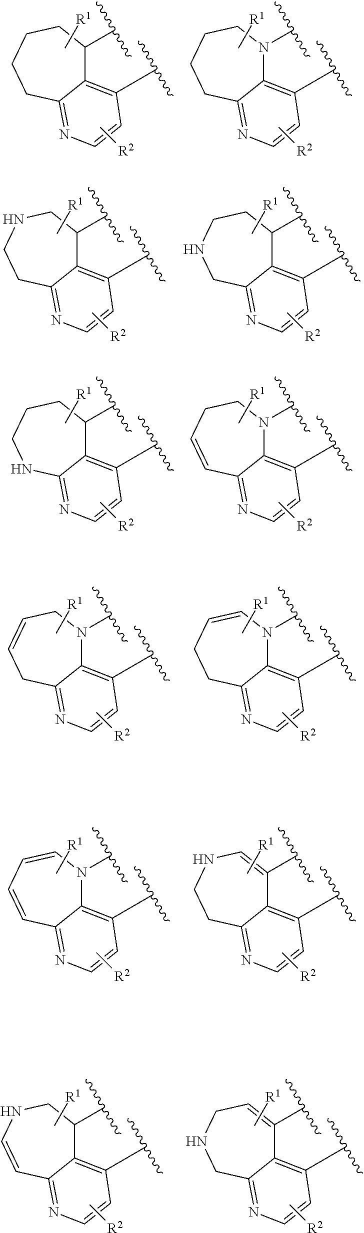

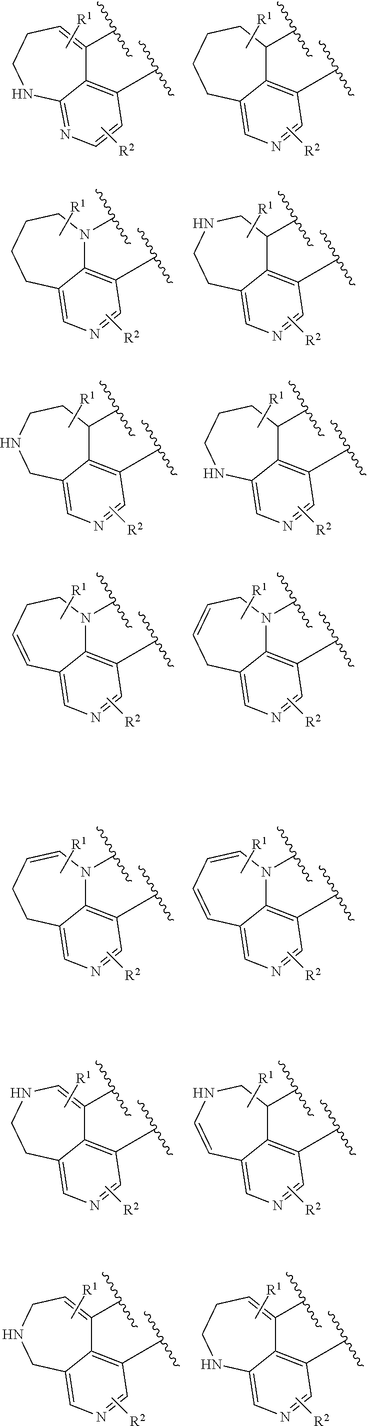

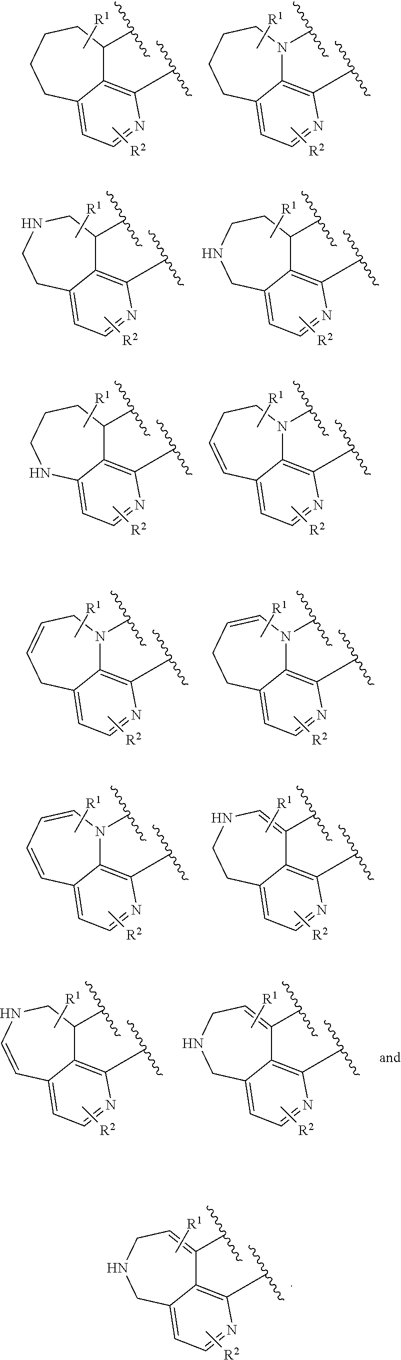

In certain embodiments

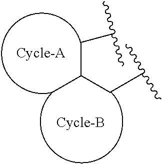 [SMILES: *CC(C)C*]

is selected from:

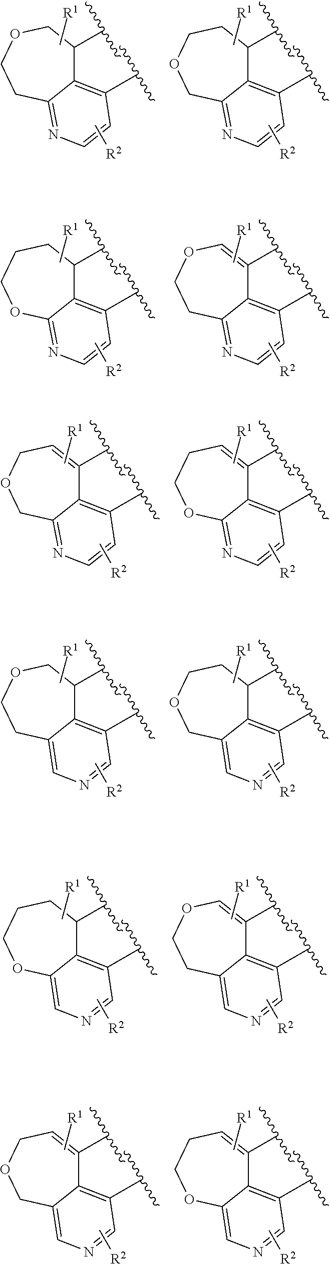

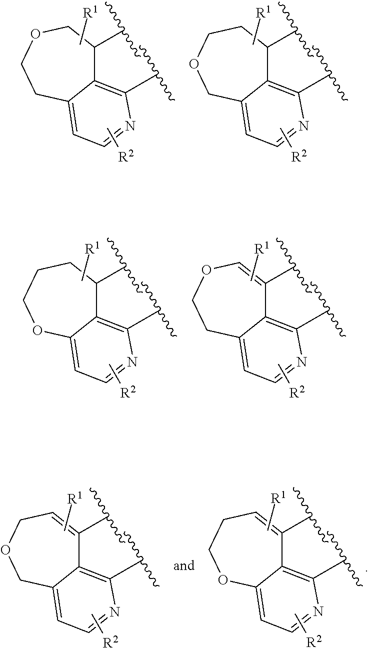

In certain embodiments

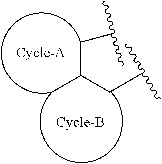 [SMILES: *CC(C)C*]

is selected from:

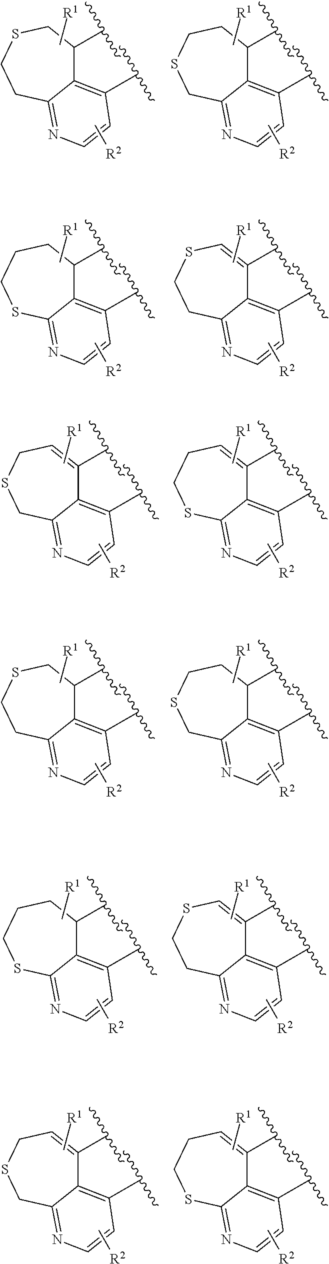

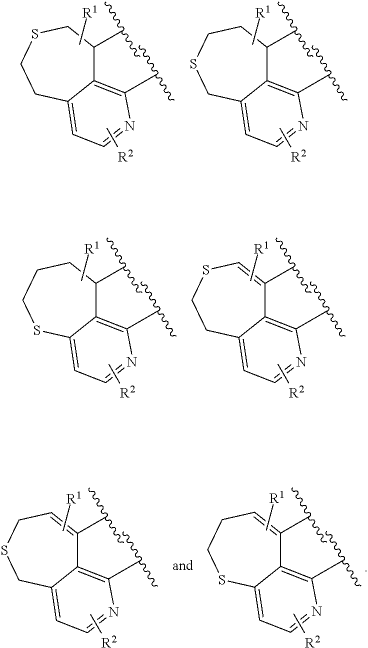

In certain embodiments

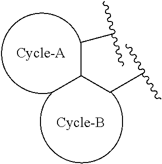 [SMILES: *CC(C)C*]

is selected from:

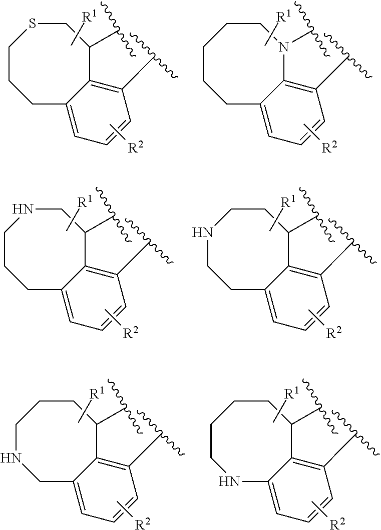

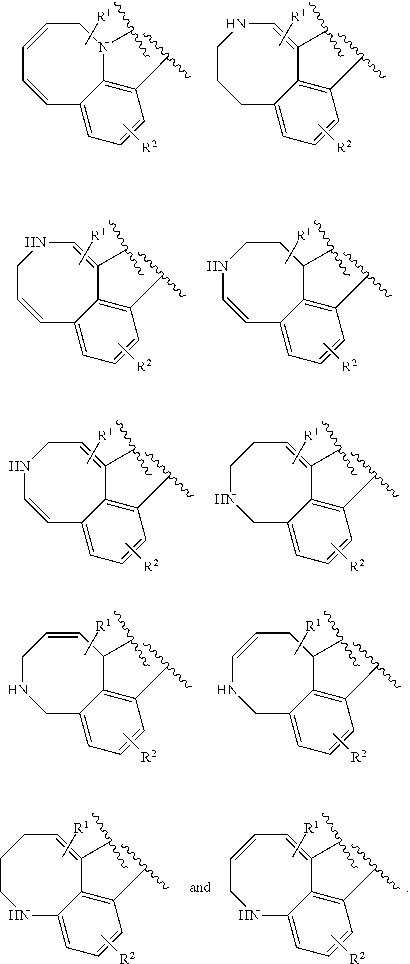

In certain embodiments

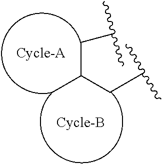 [SMILES: *CC(C)C*]

is selected from:

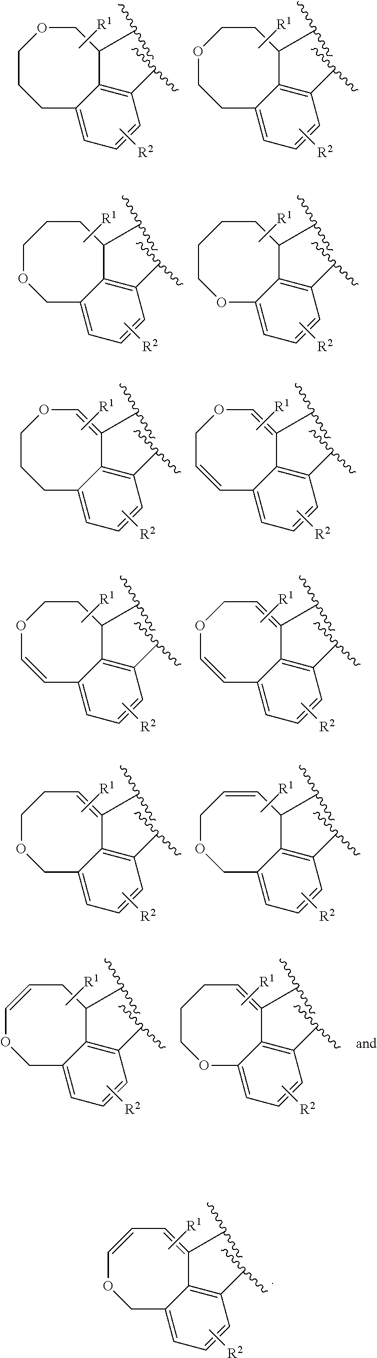

In certain embodiments

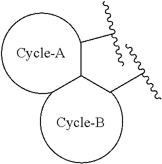 [SMILES: *CC(C)C*]

is selected from:

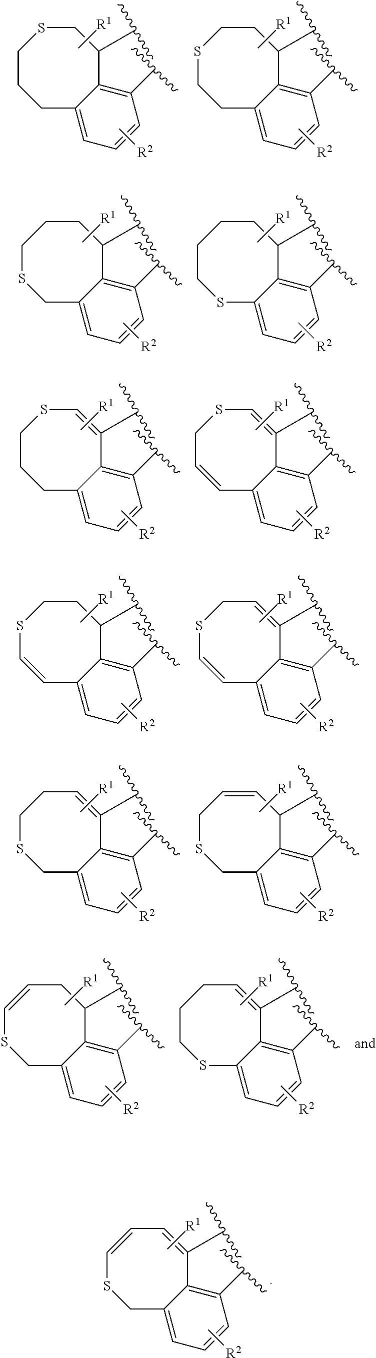

In certain embodiments

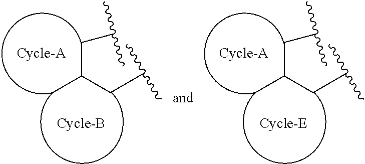

are selected from:

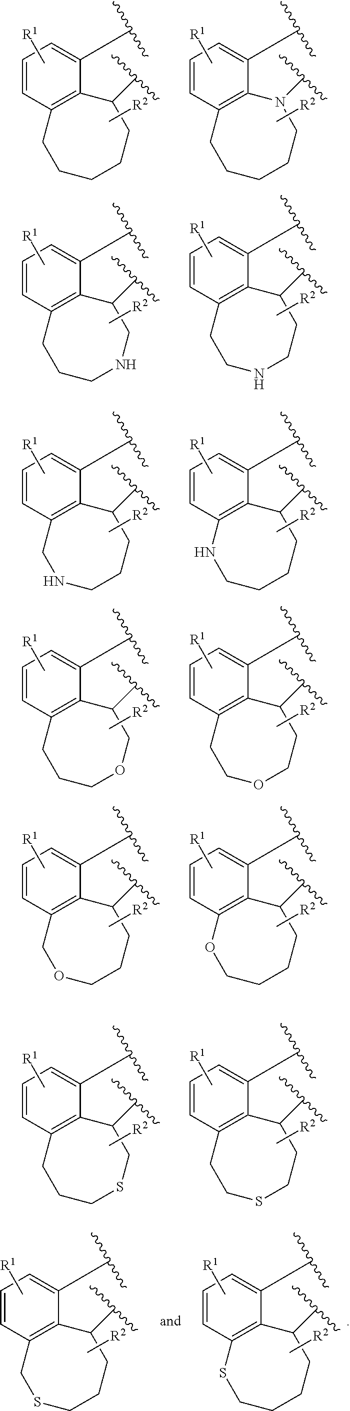

In certain embodiments

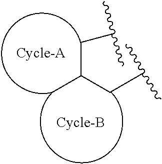 [SMILES: *CC(C)C*]

is selected from:

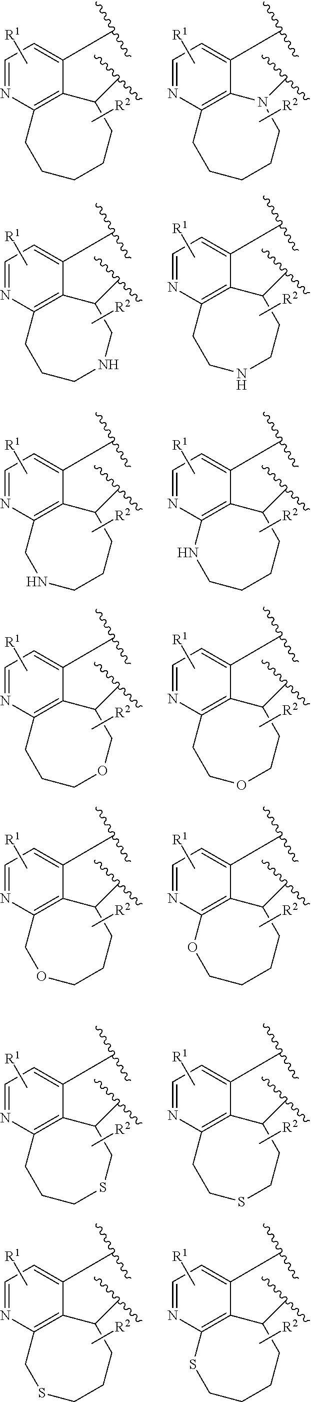

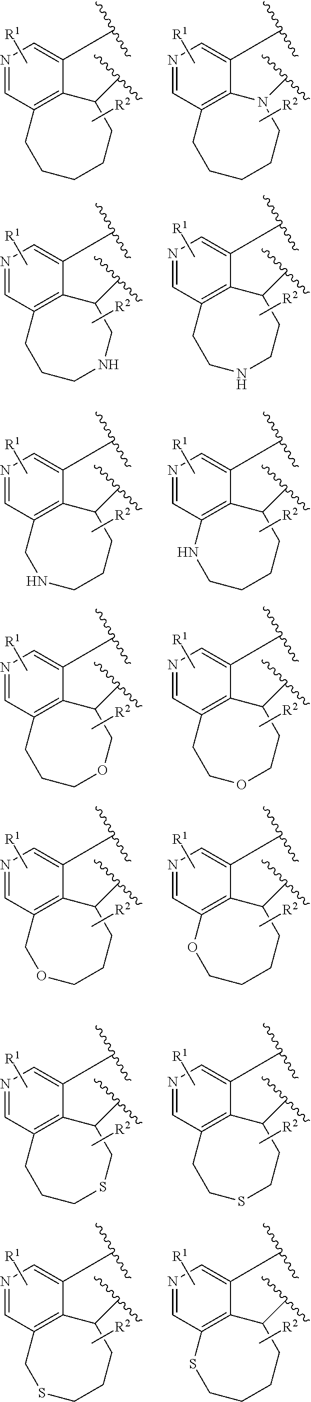

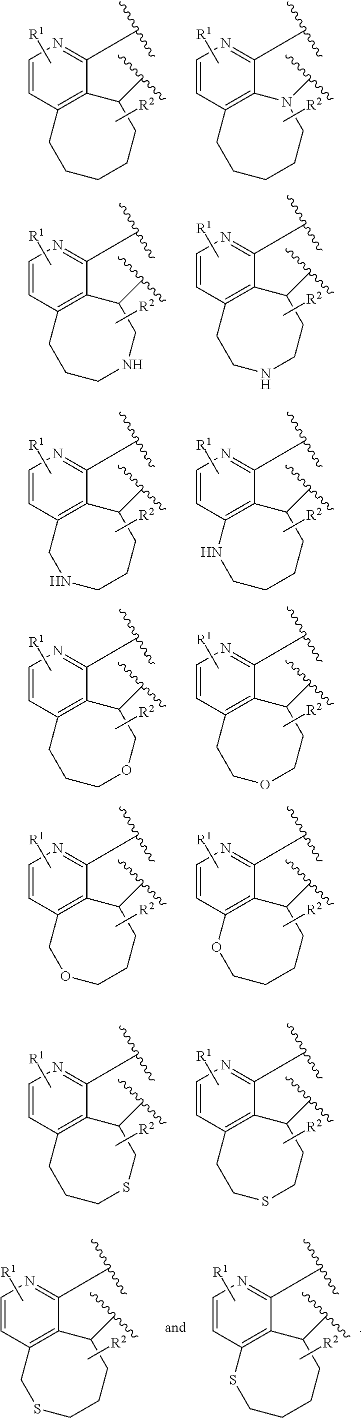

In certain embodiments

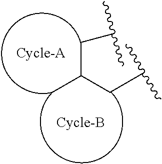 [SMILES: *CC(C)C*]

is selected from:

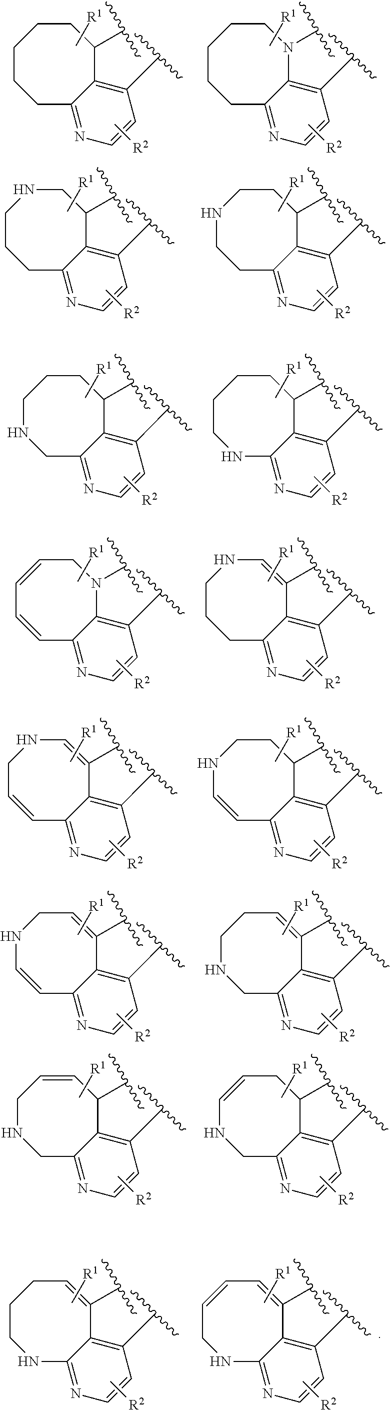

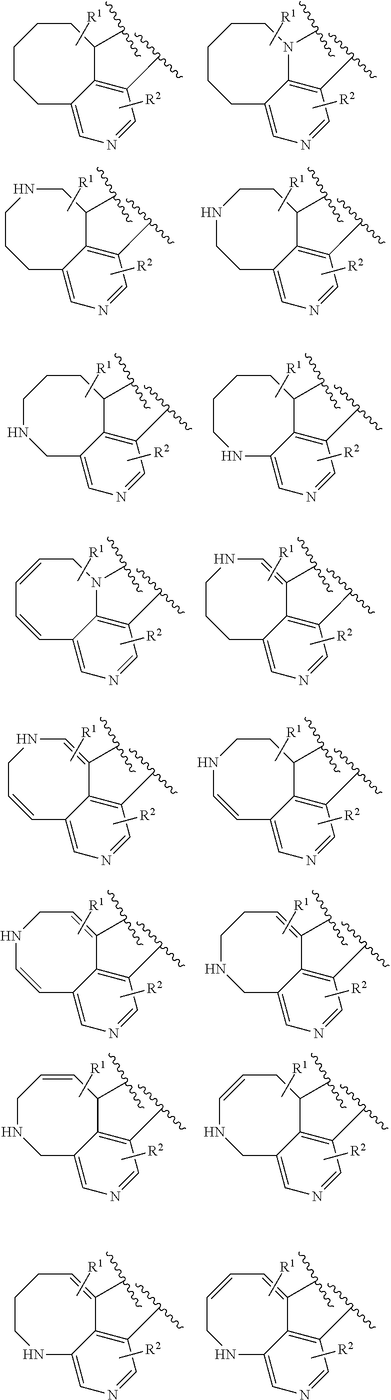

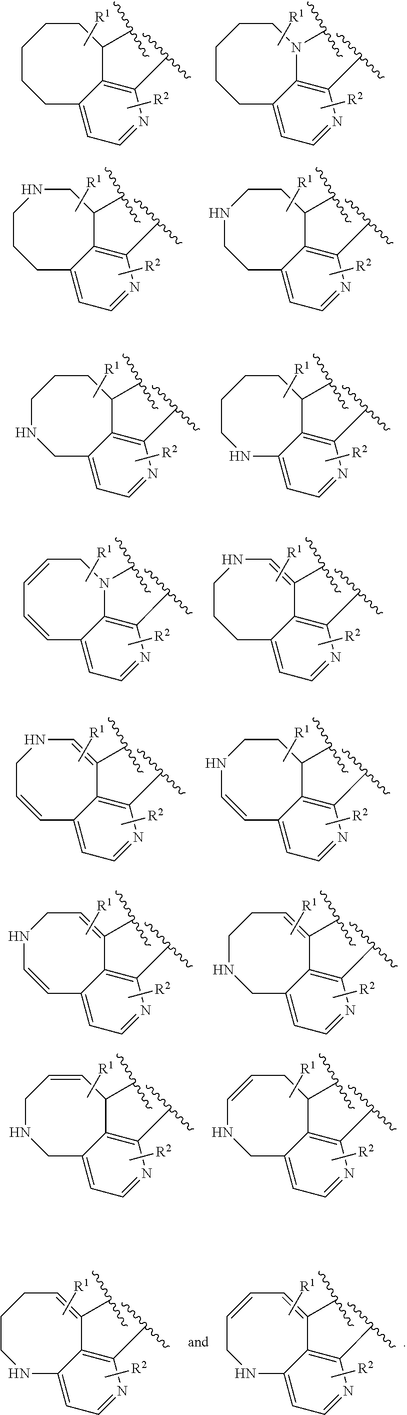

In certain embodiments

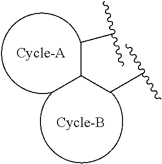 [SMILES: *CC(C)C*]

is selected from:

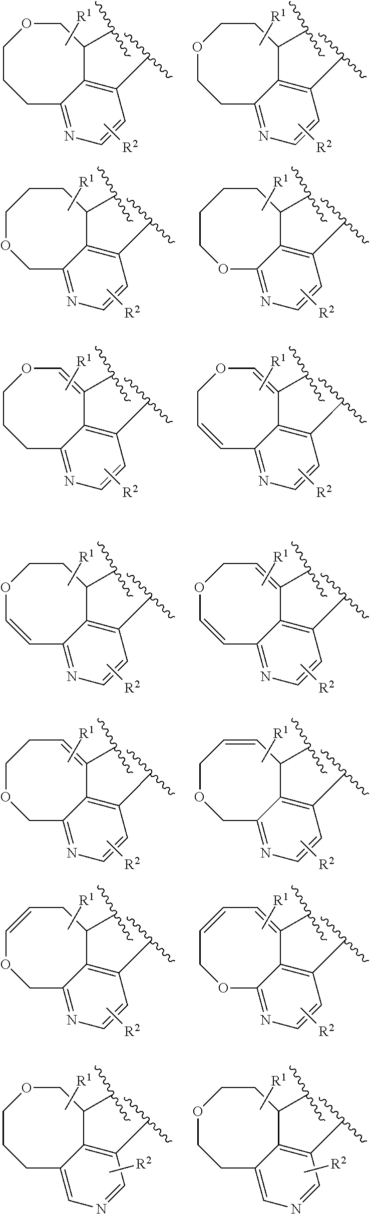

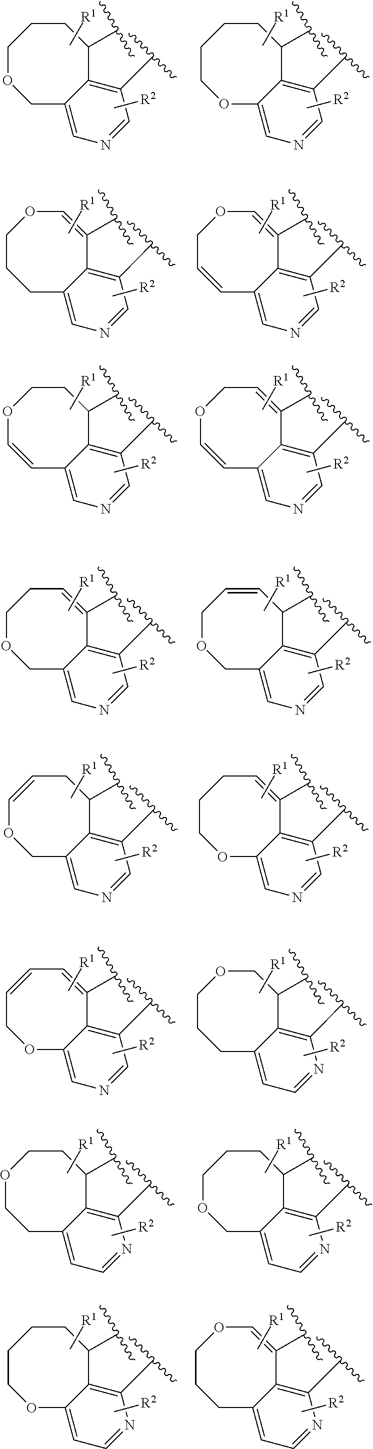

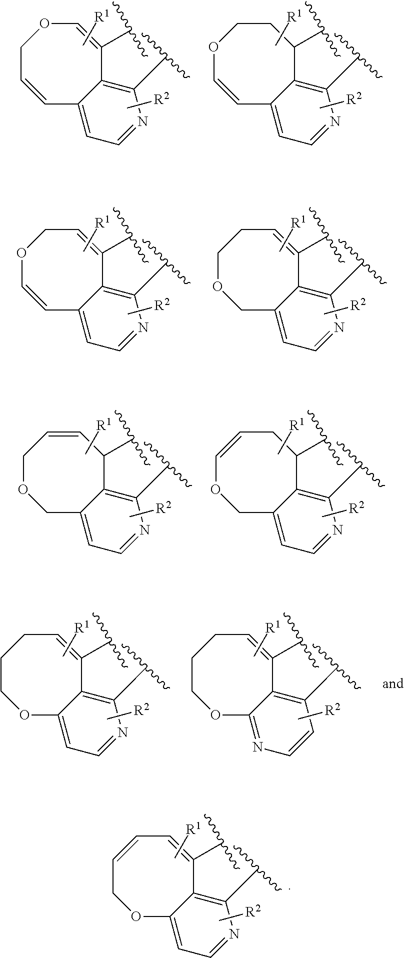

In certain embodiments

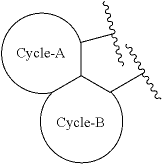 [SMILES: *CC(C)C*]

is selected from:

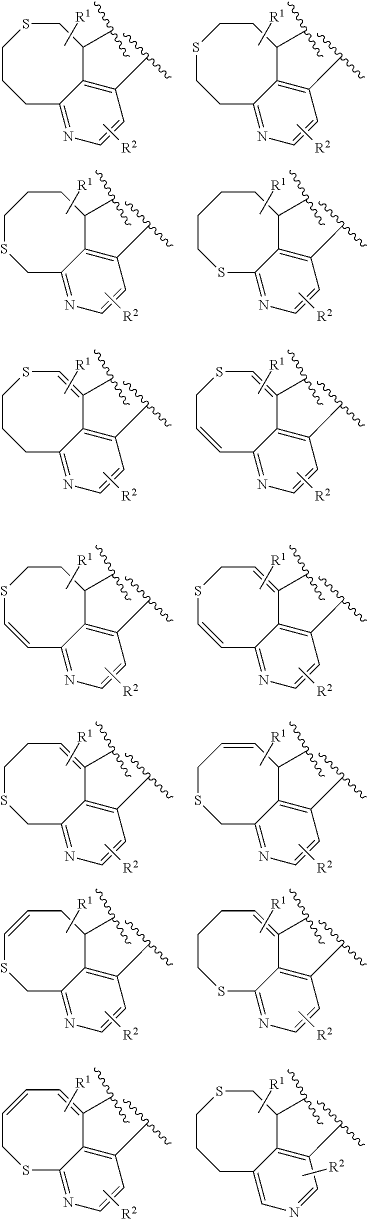

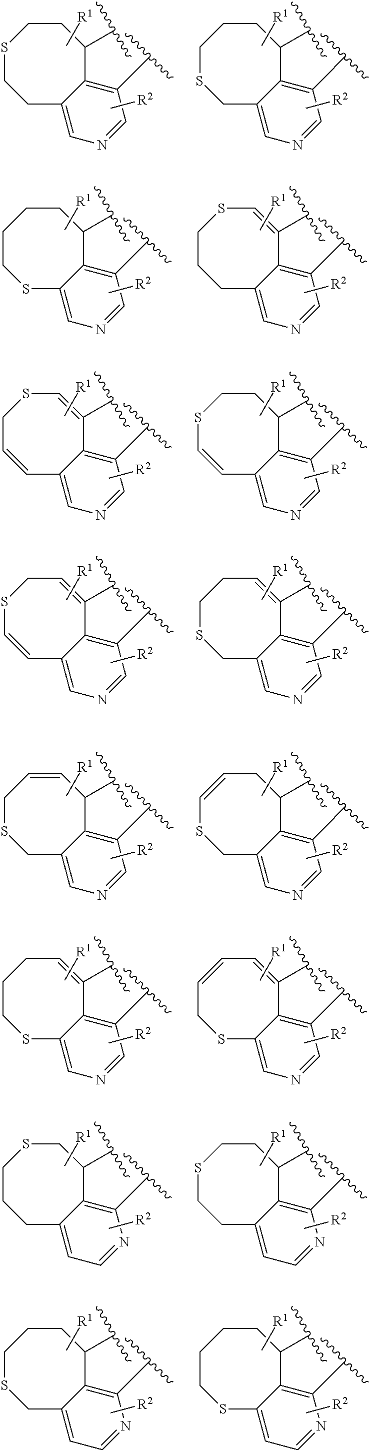

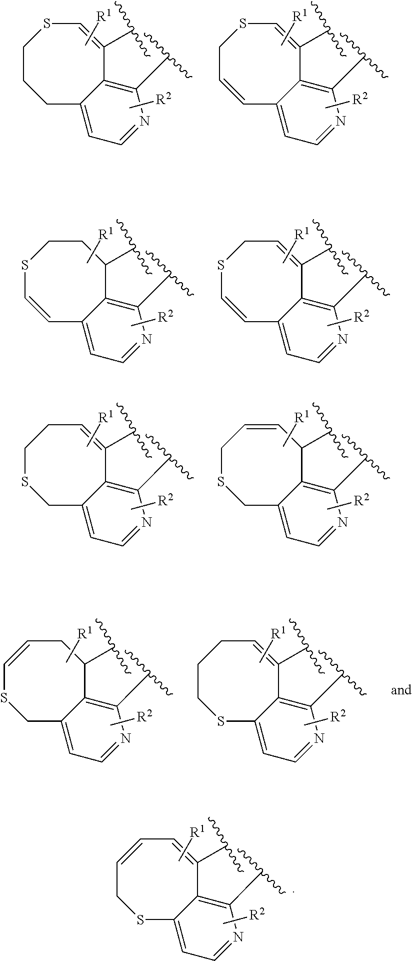

In certain embodiments

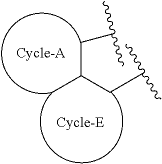 [SMILES: *CC(C)C*]

is selected from the following:

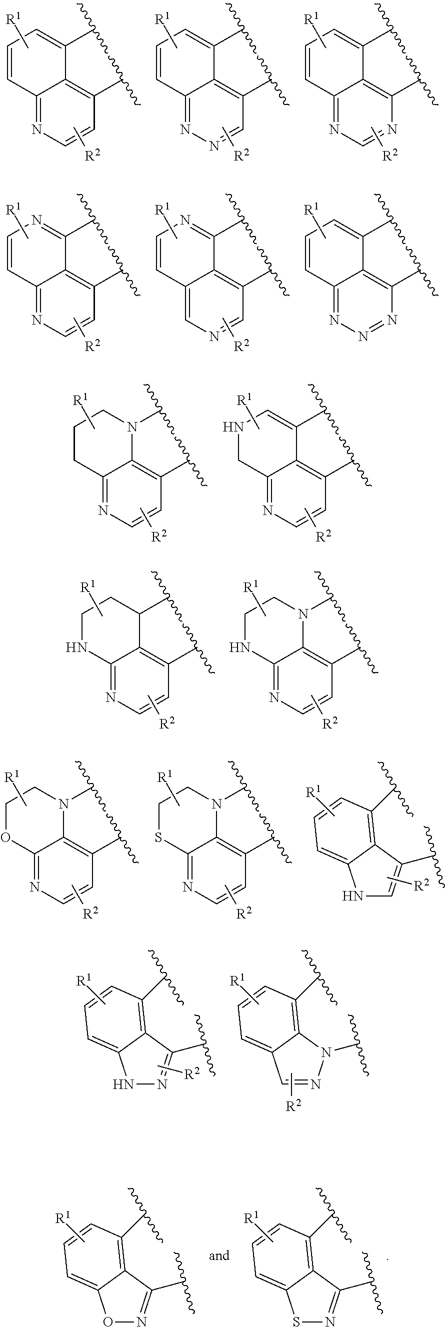

In certain embodiments

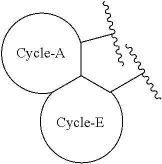 [SMILES: *CC(C)C*]

is selected from the following:

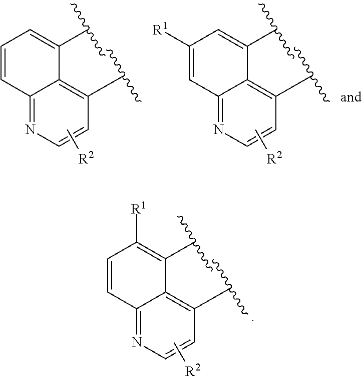

In certain embodiments

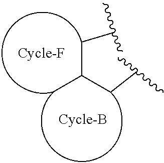 [SMILES: *CC(C)C*]

is selected from the following:

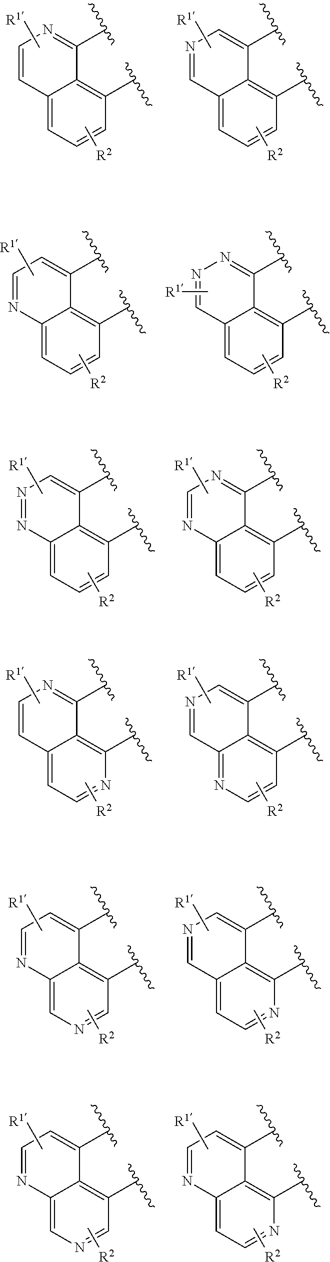

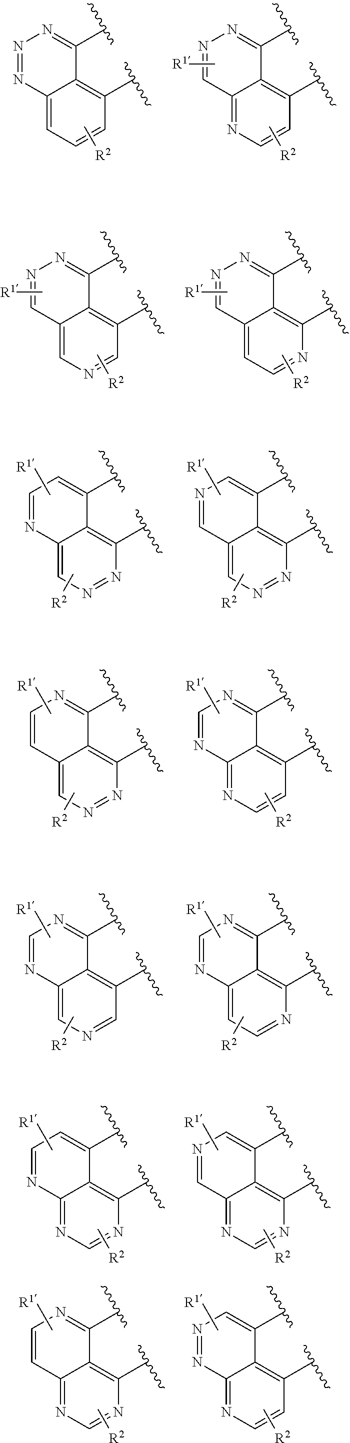

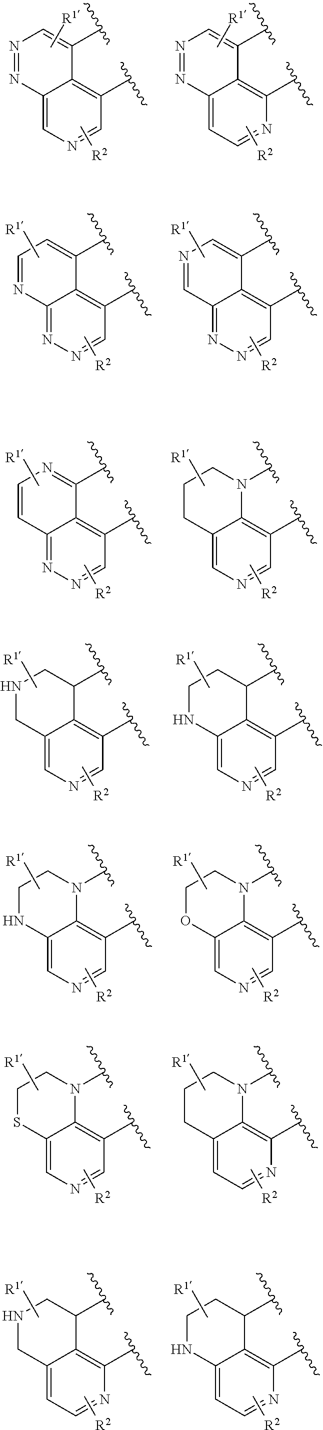

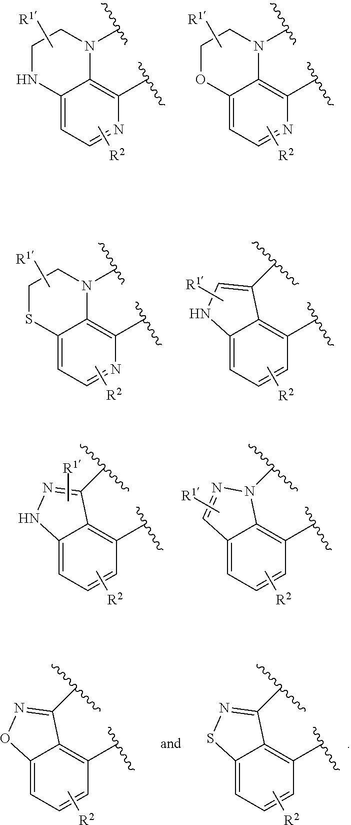

In certain embodiments

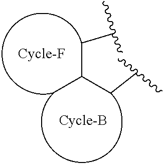 [SMILES: *CC(C)C*]

is selected from:

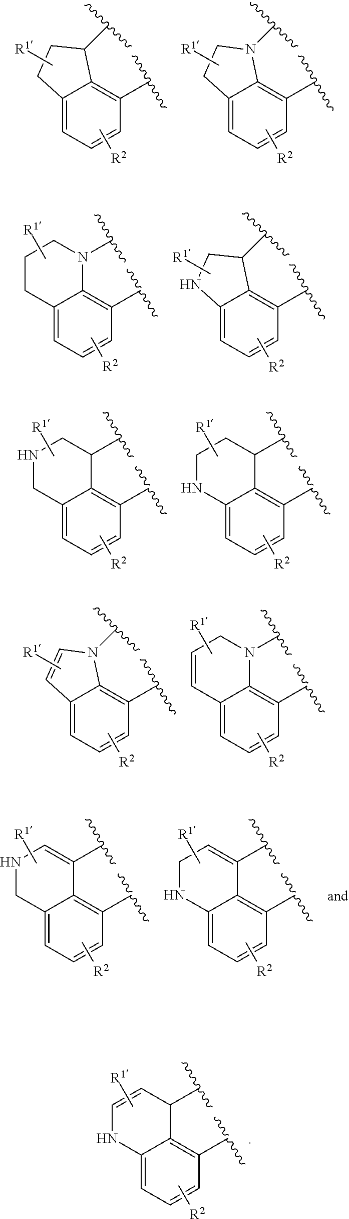

In certain embodiments

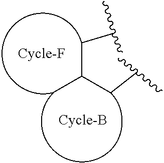 [SMILES: *CC(C)C*]

is selected from:

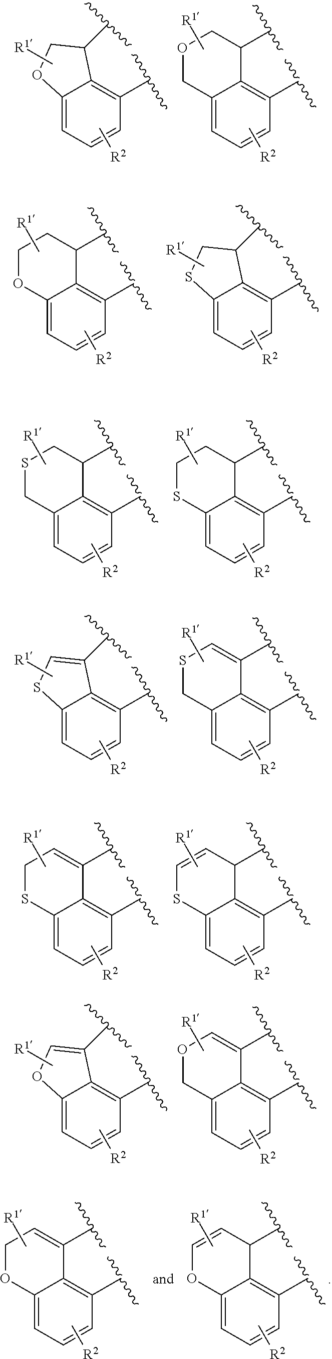

In certain embodiments

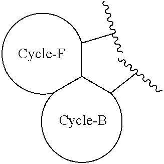 [SMILES: *CC(C)C*]

is selected from:

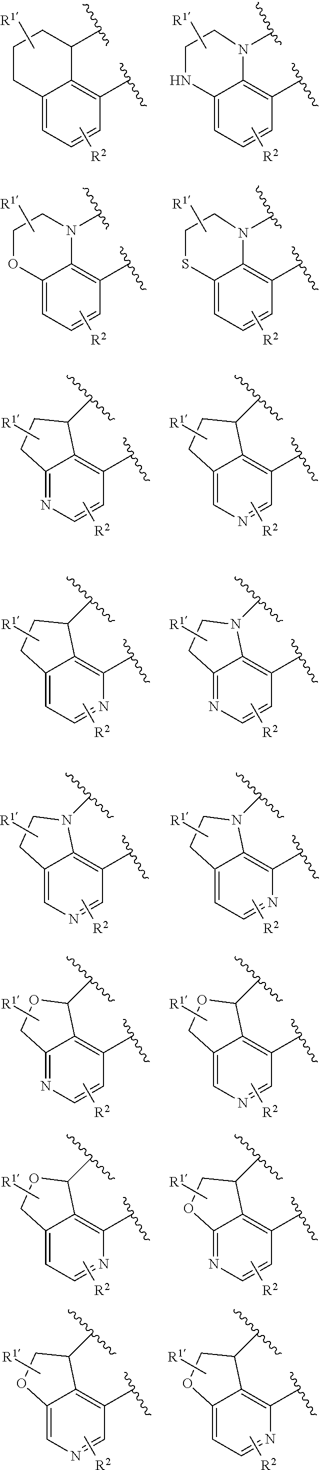

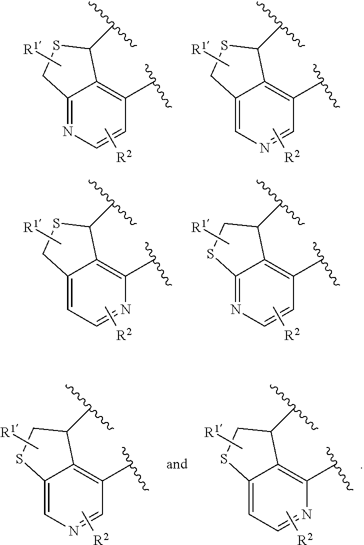

In certain embodiments

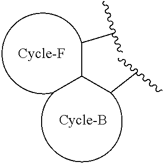 [SMILES: *CC(C)C*]

is selected from:

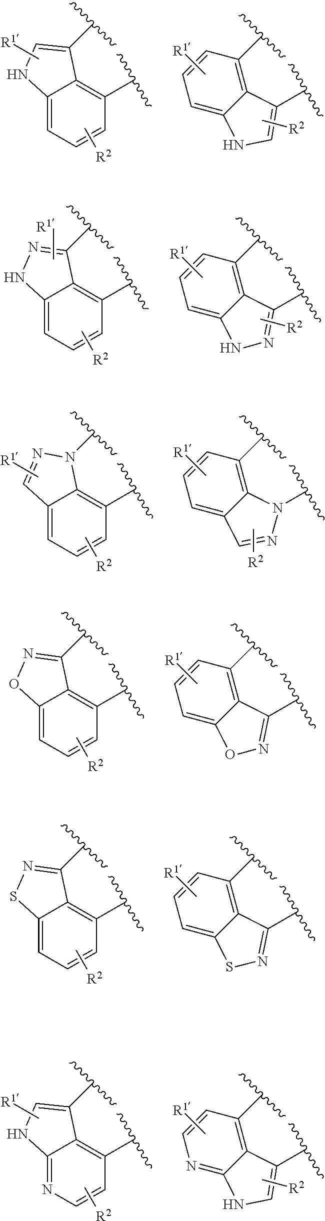

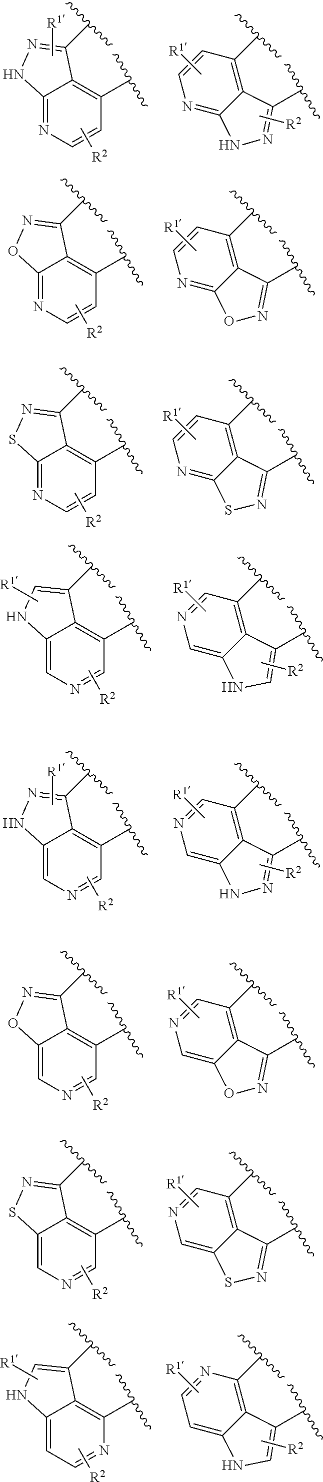

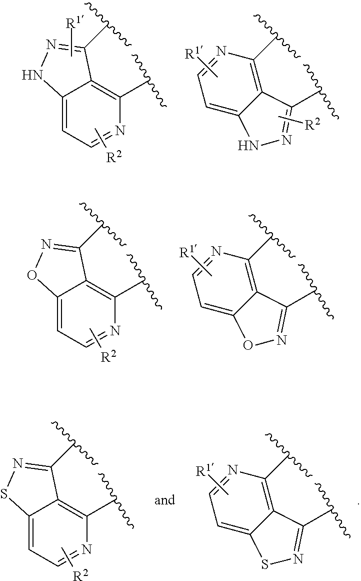

In certain embodiments

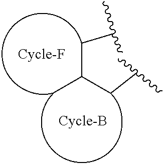 [SMILES: *CC(C)C*]

is selected from:

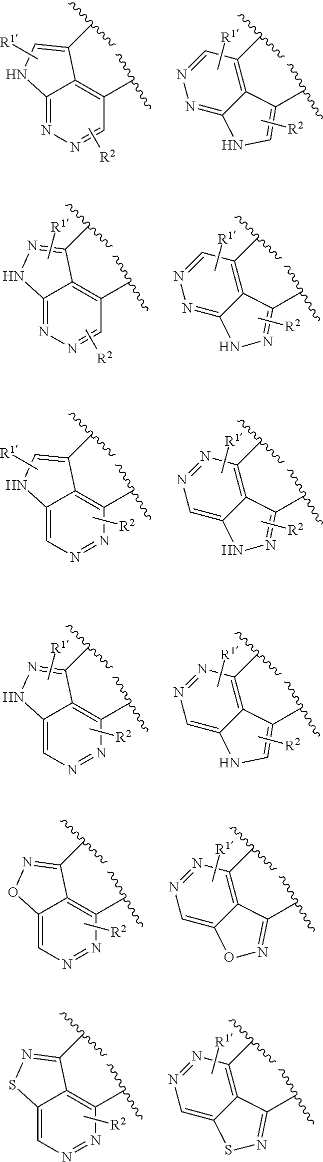

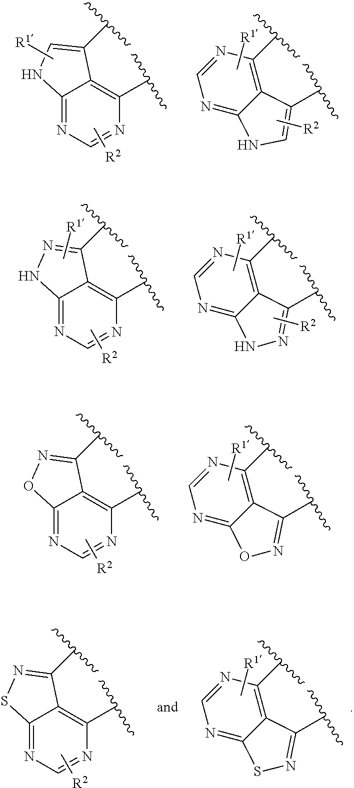

In certain embodiments

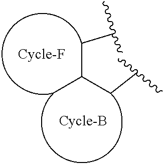 [SMILES: *CC(C)C*]

is selected from:

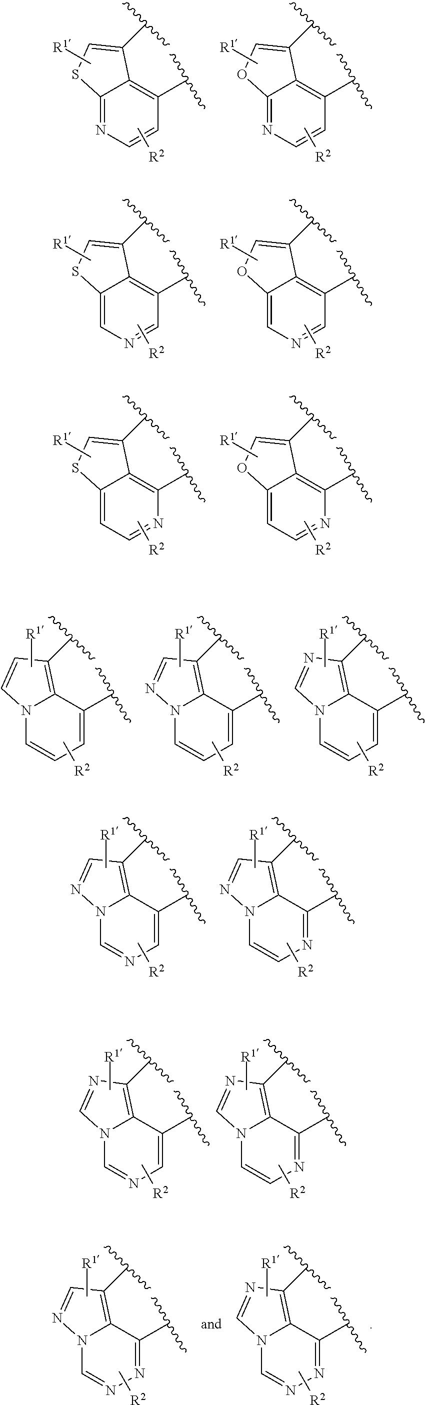

In certain embodiments

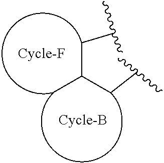 [SMILES: *CC(C)C*]

is selected from.

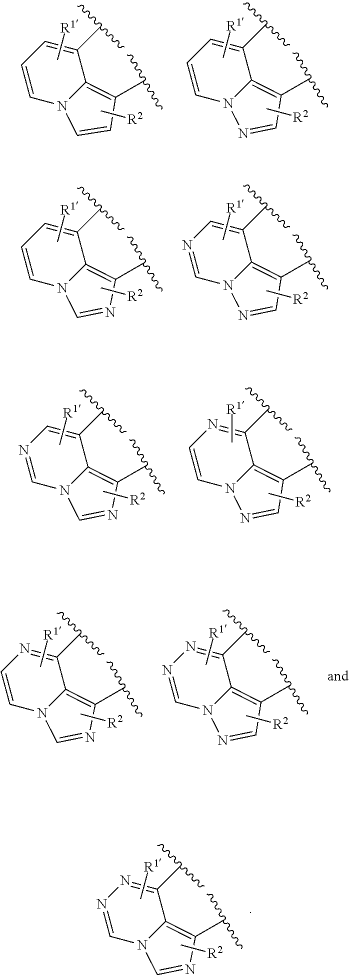

In certain embodiment

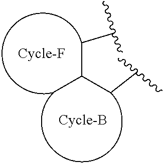 [SMILES: *CC(C)C*]

is selected from:

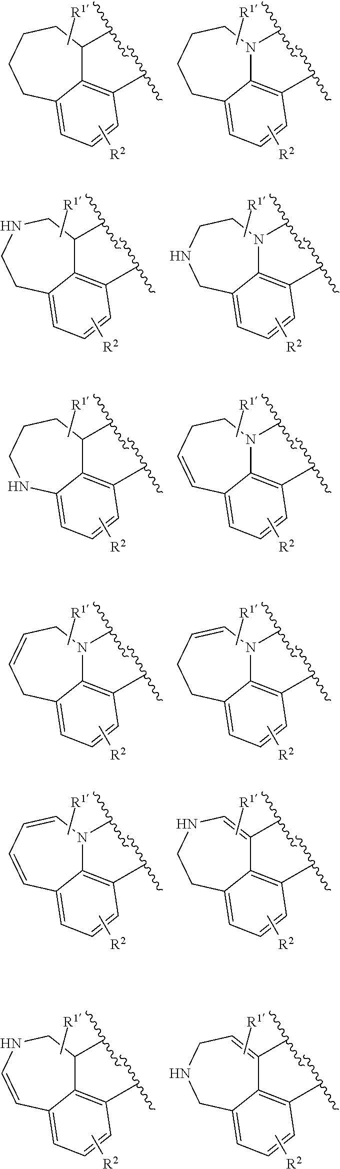

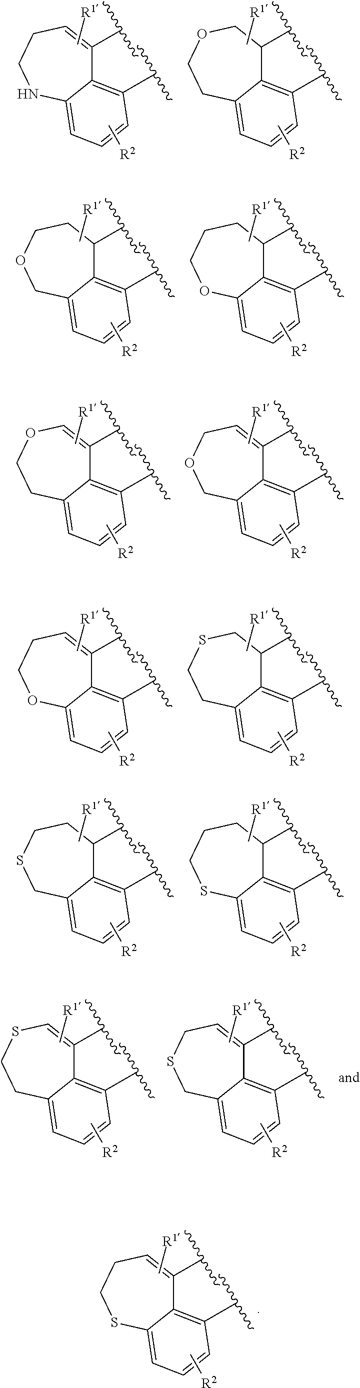

In certain embodiments

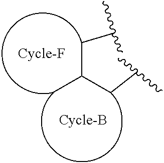 [SMILES: *CC(C)C*]

is selected from:

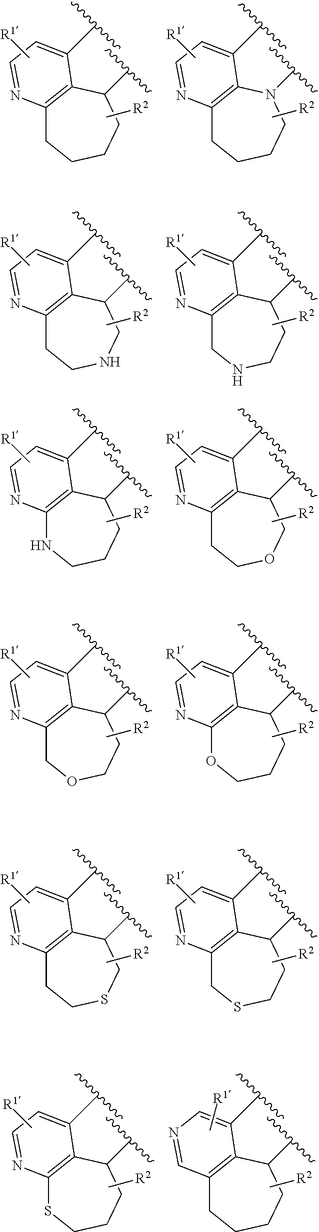

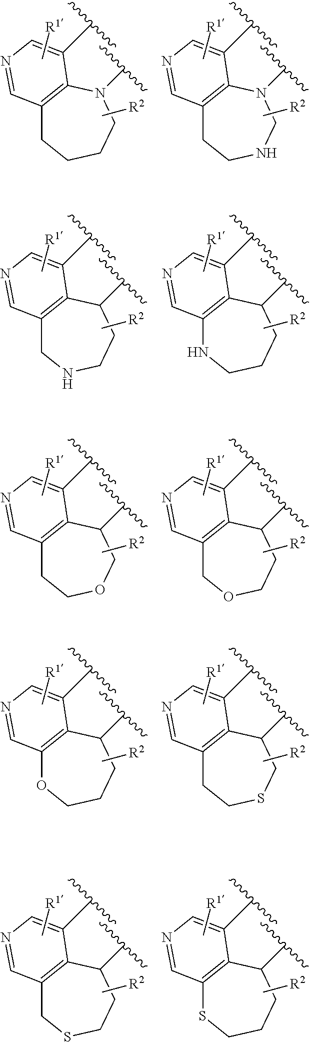

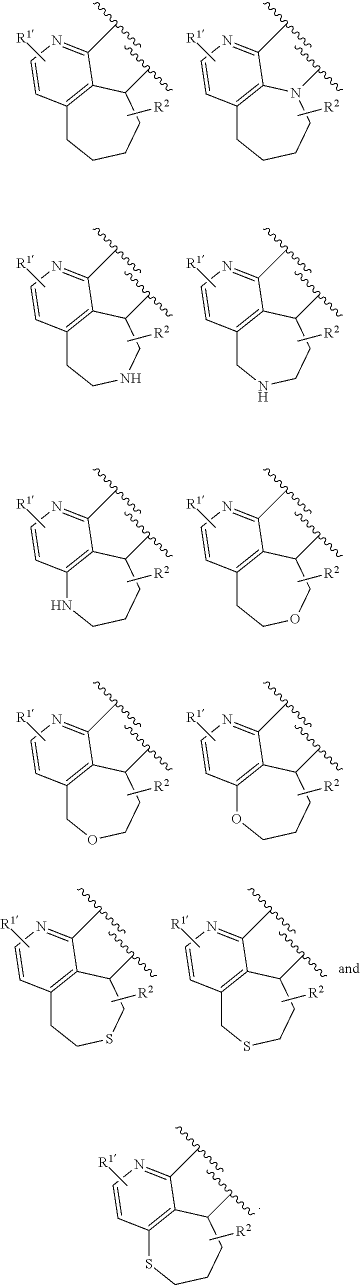

In certain embodiments

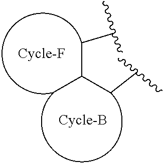 [SMILES: *CC(C)C*]

is selected from:

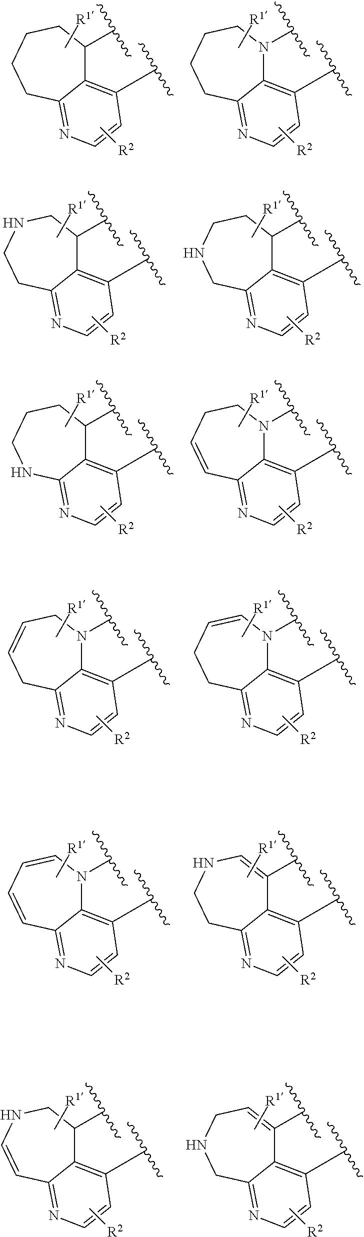

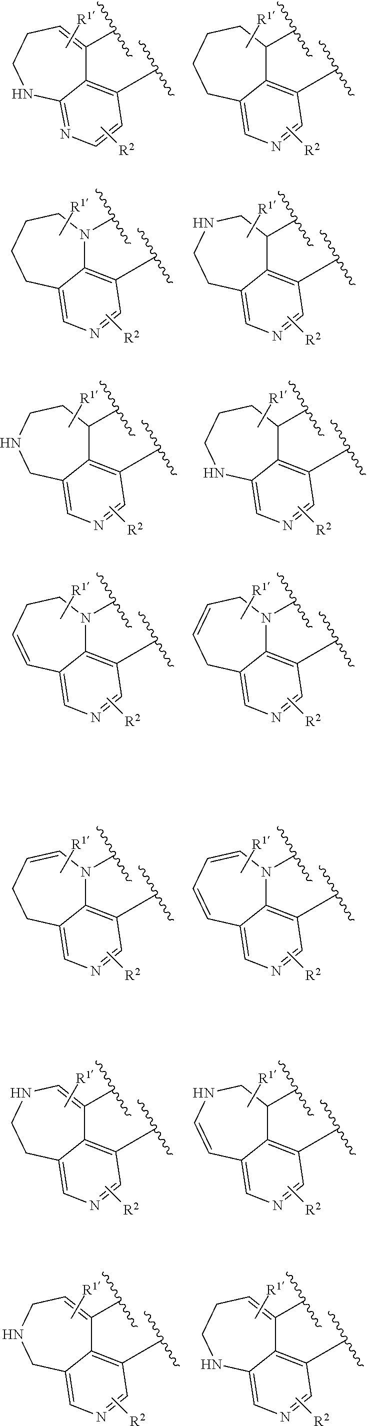

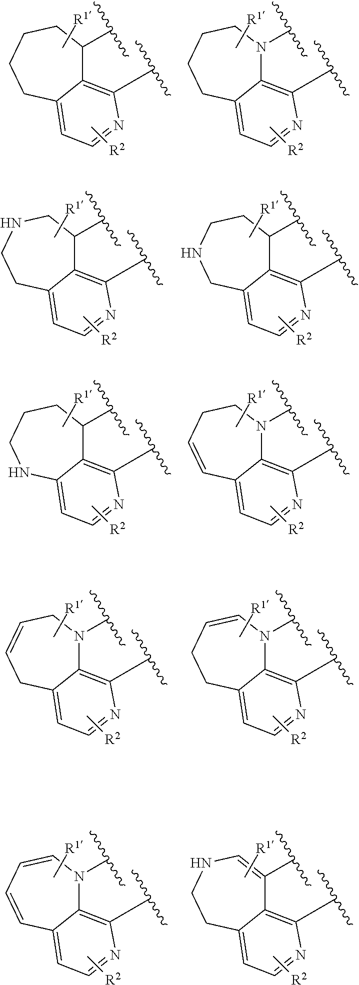

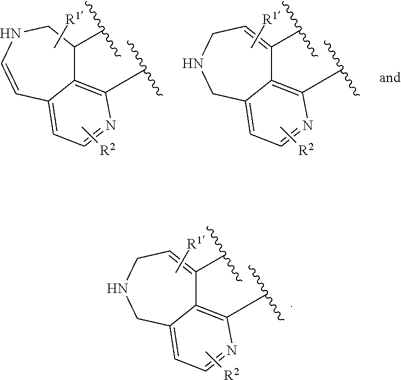

In certain embodiments

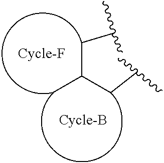 [SMILES: *CC(C)C*]

is selected from:

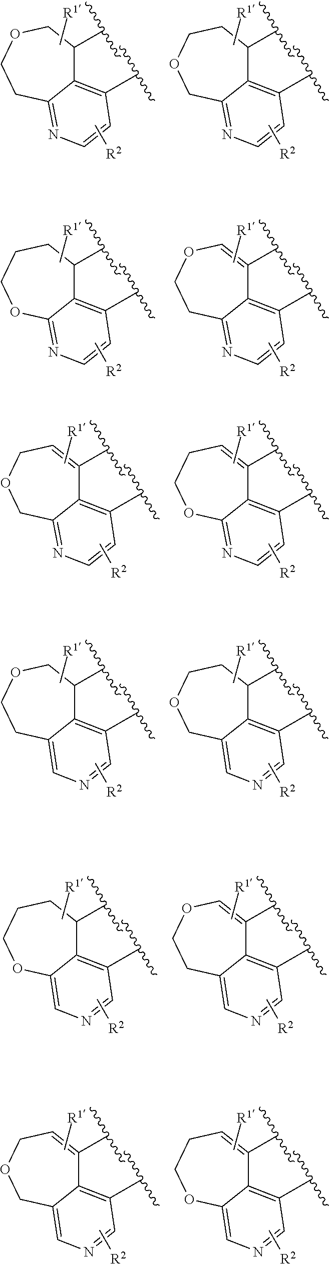

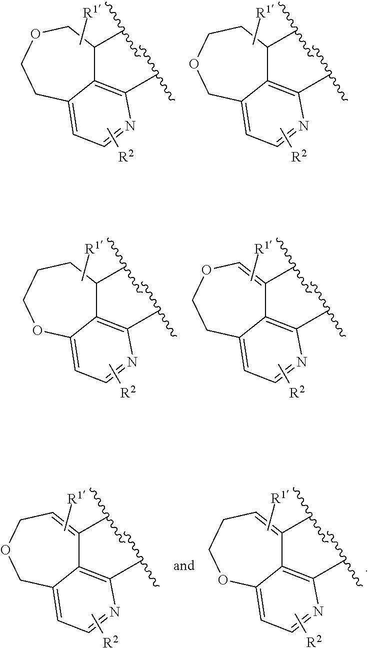

In certain embodiments

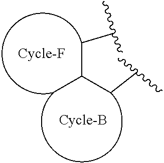 [SMILES: *CC(C)C*]

is selected from:

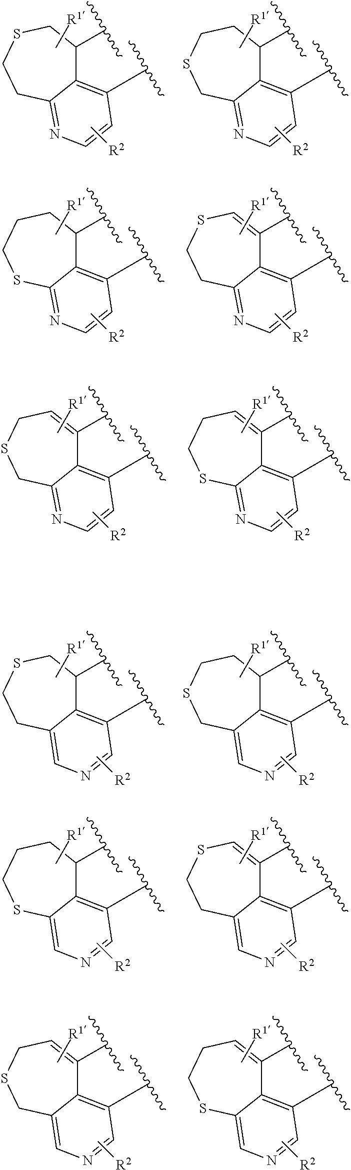

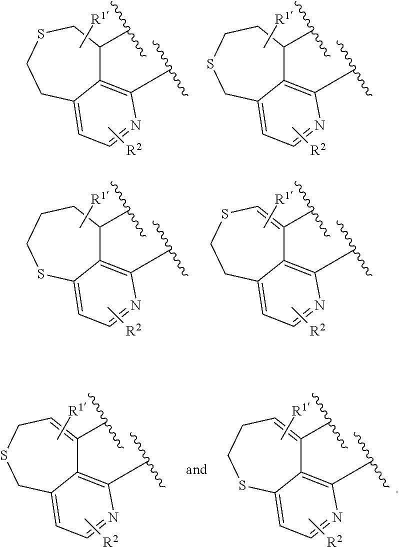

In certain embodiments

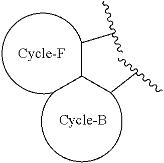 [SMILES: *CC(C)C*]

is selected from:

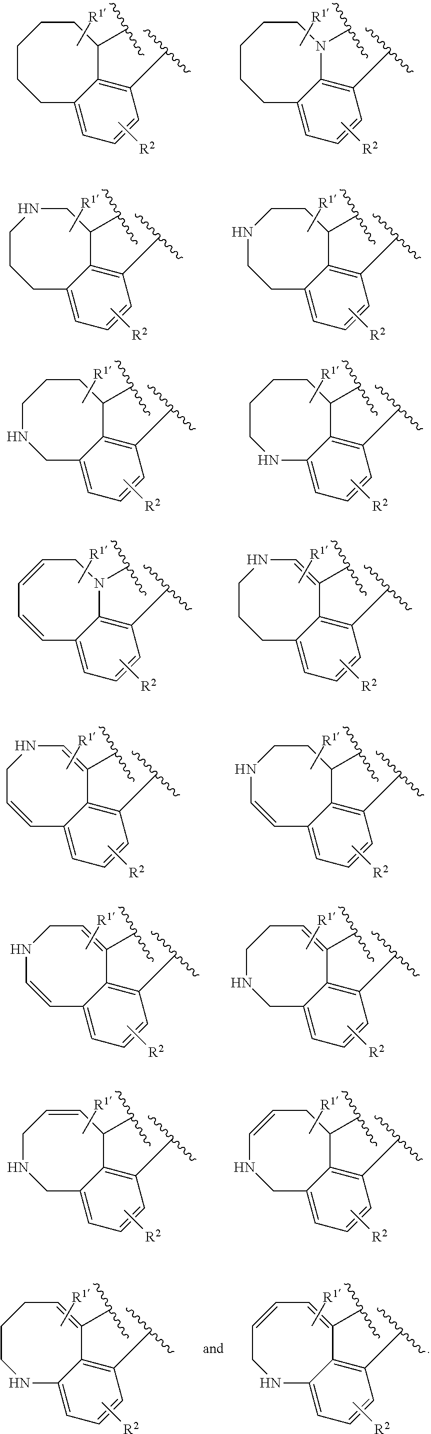

In certain embodiments

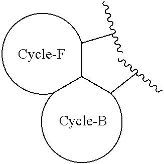 [SMILES: *CC(C)C*]

is selected from:

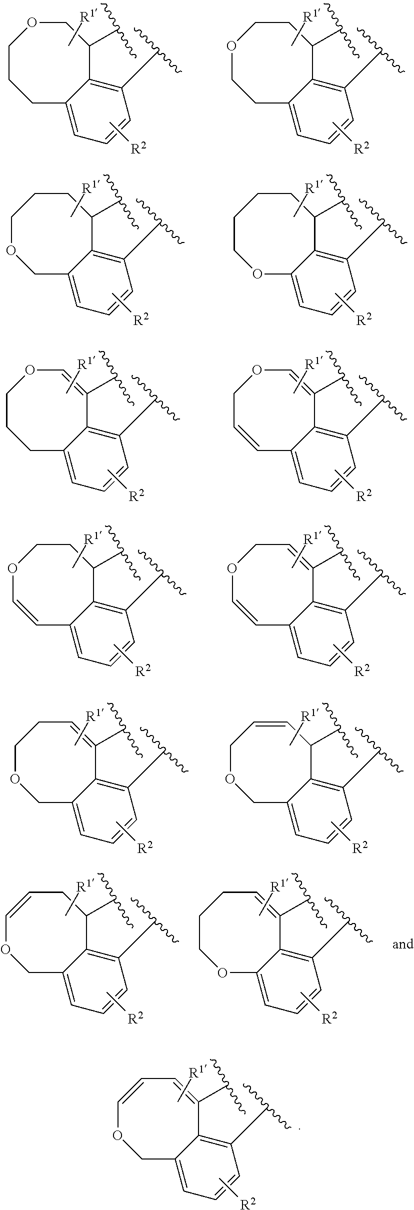

In certain embodiments

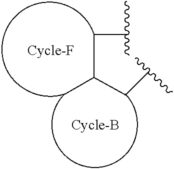 [SMILES: *CC(C)C*]

is selected from:

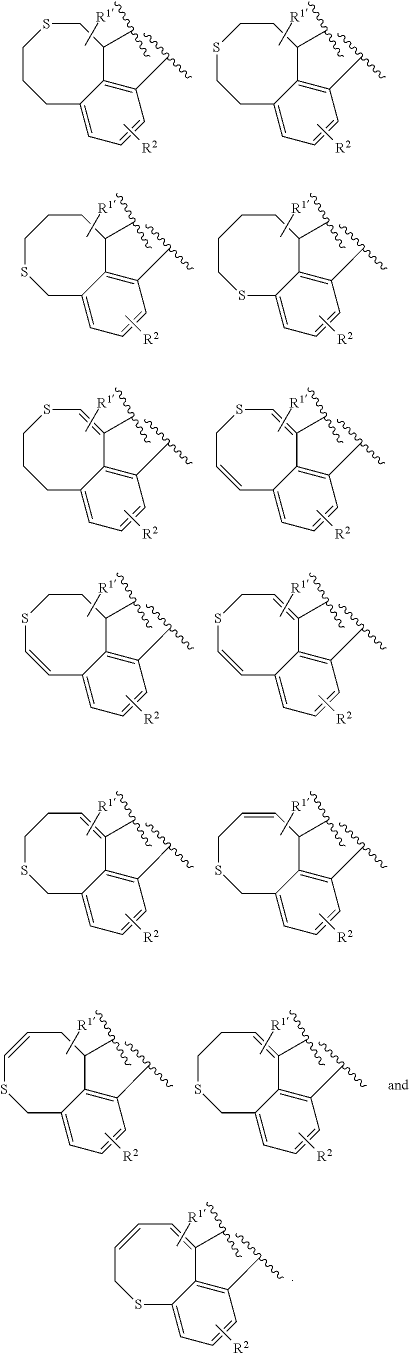

In certain embodiments

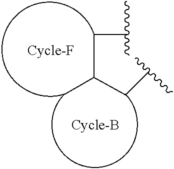 [SMILES: *CC(C)C*]

is selected from:

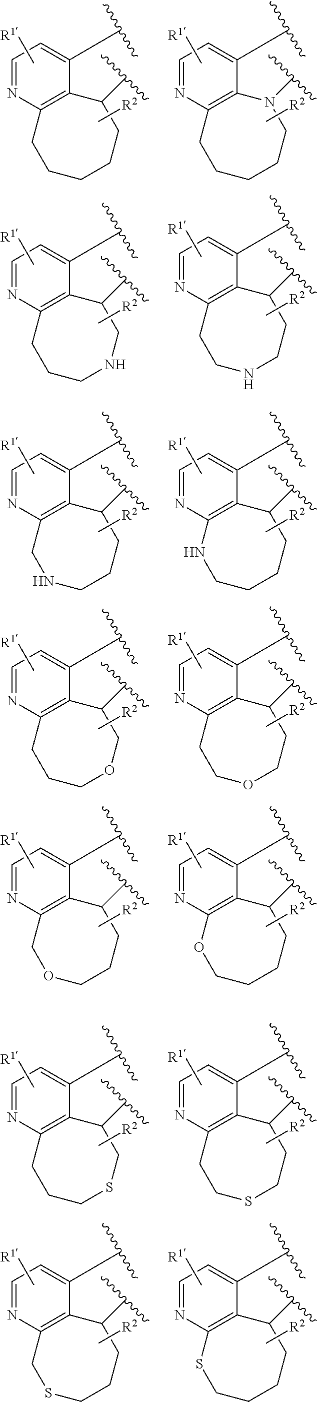

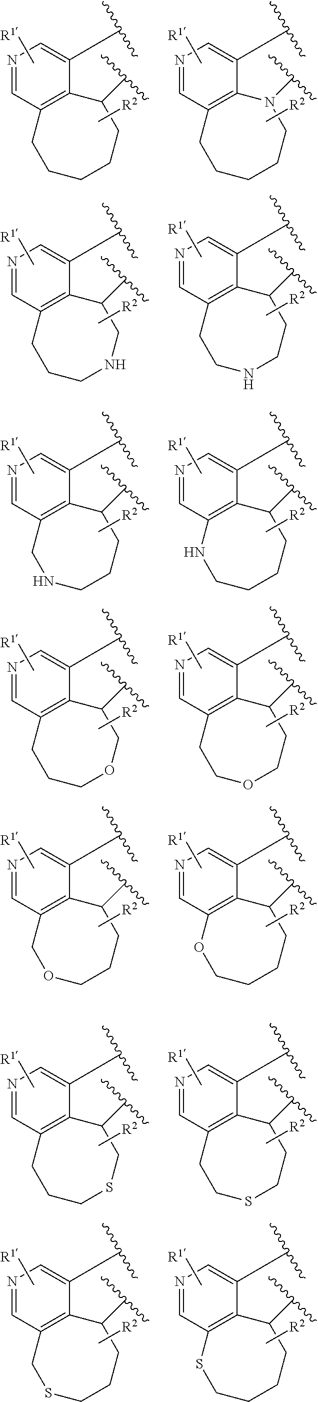

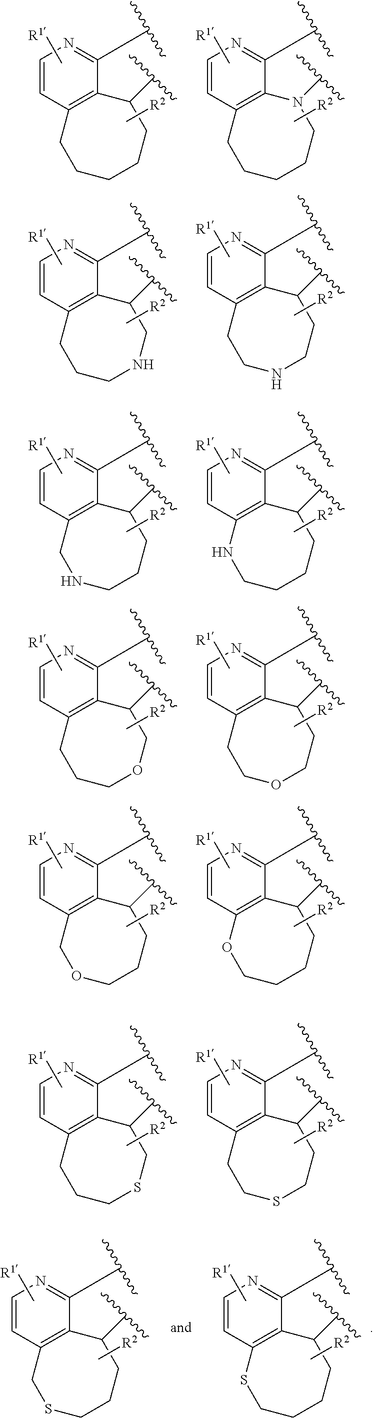

In certain embodiments

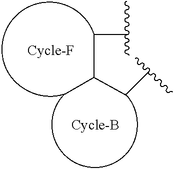 [SMILES: *CC(C)C*]

is selected from:

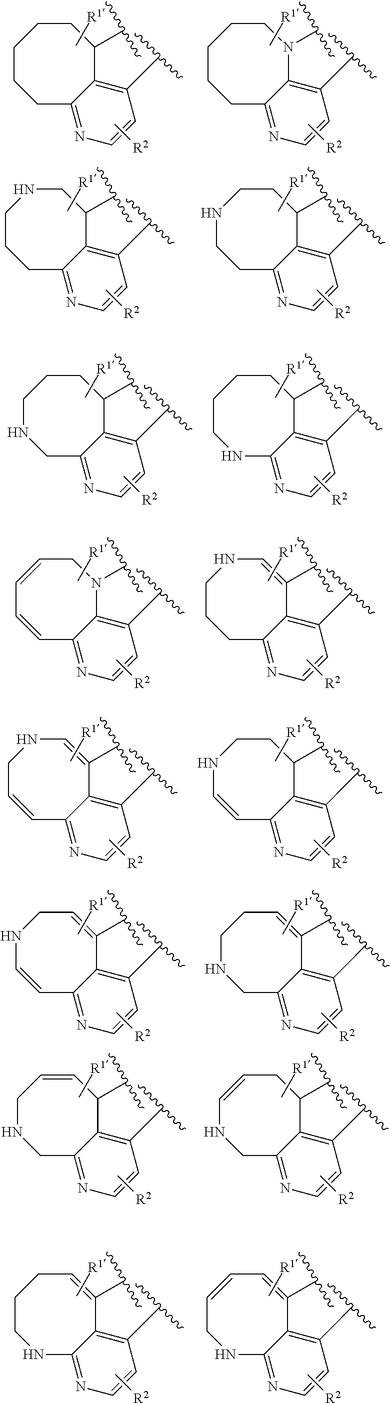

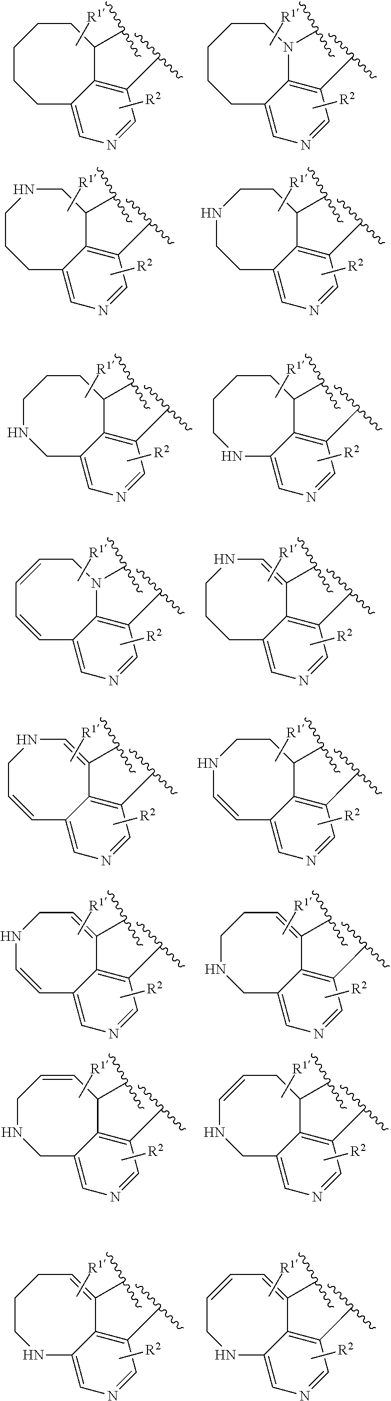

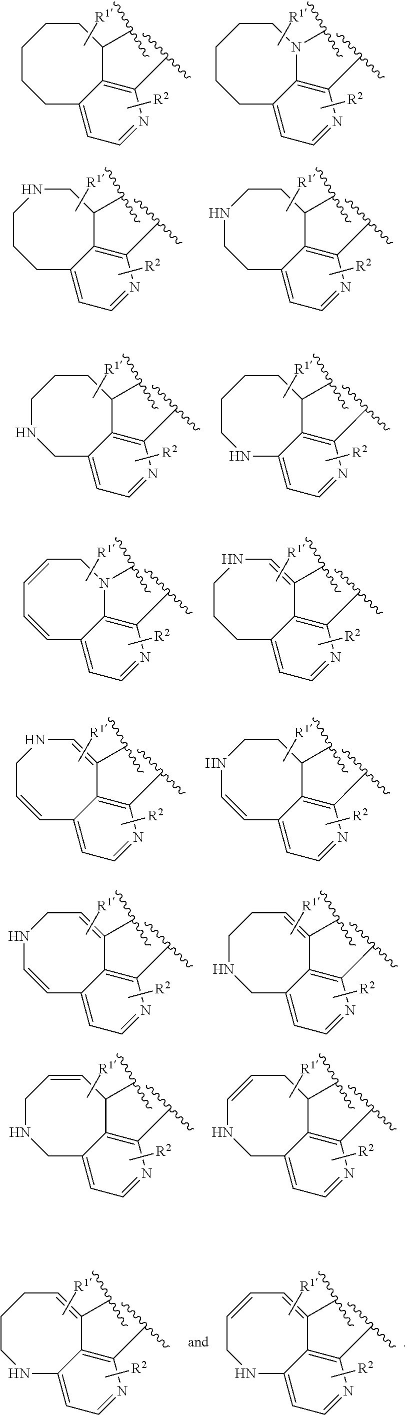

In certain embodiments

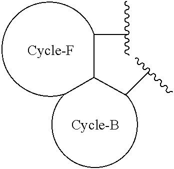 [SMILES: *CC(C)C*]

is selected from:

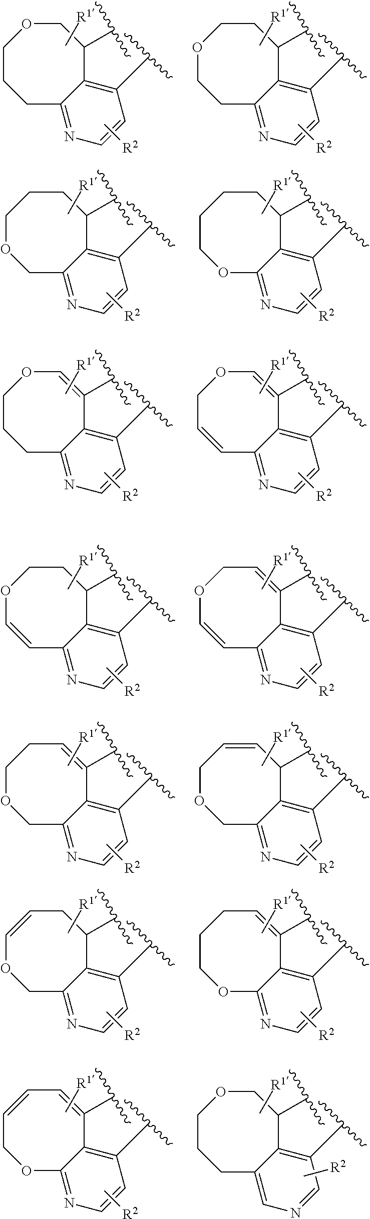

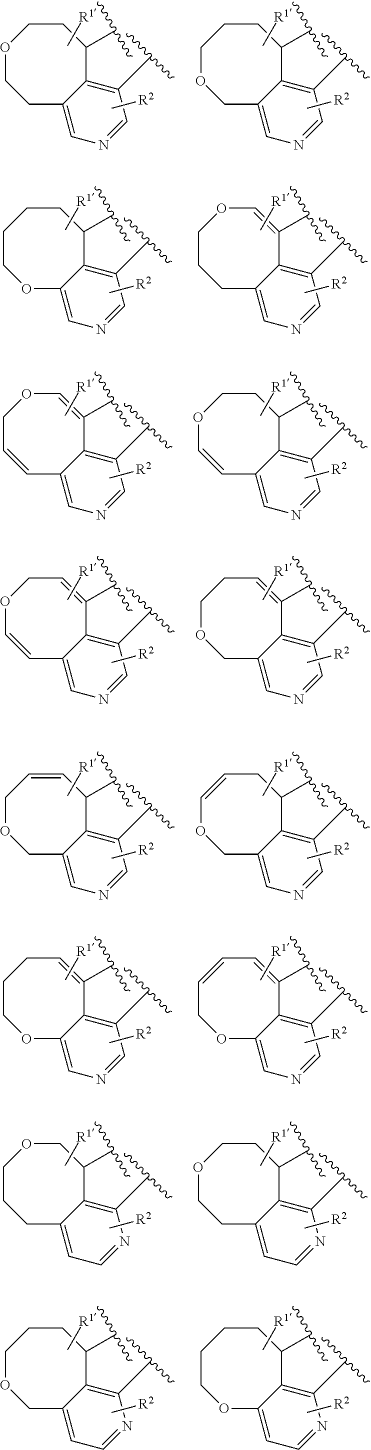

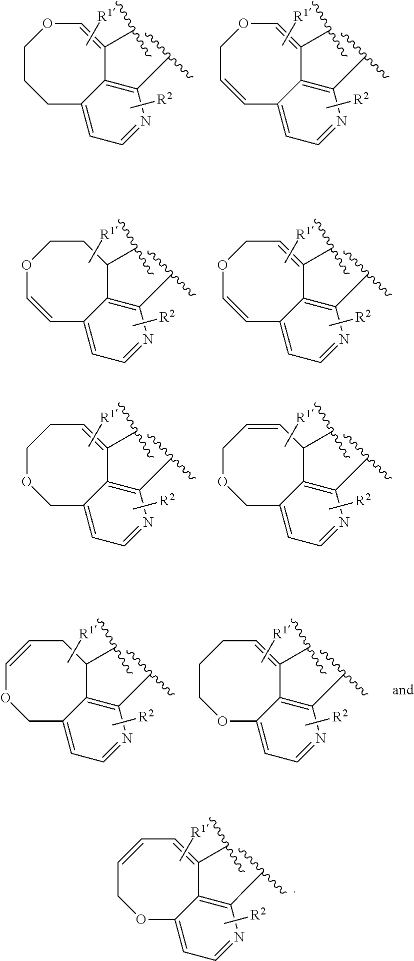

In certain embodiments

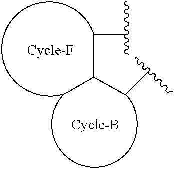 [SMILES: *CC(C)C*]

is selected from:

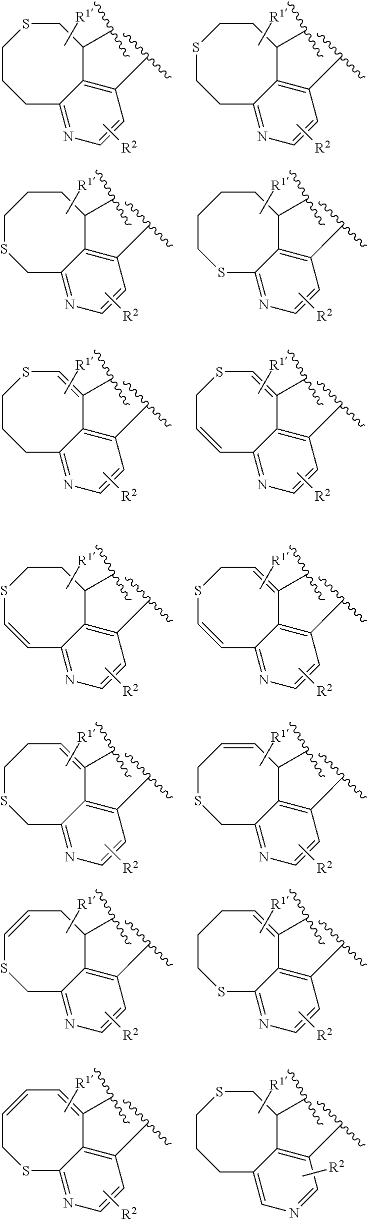

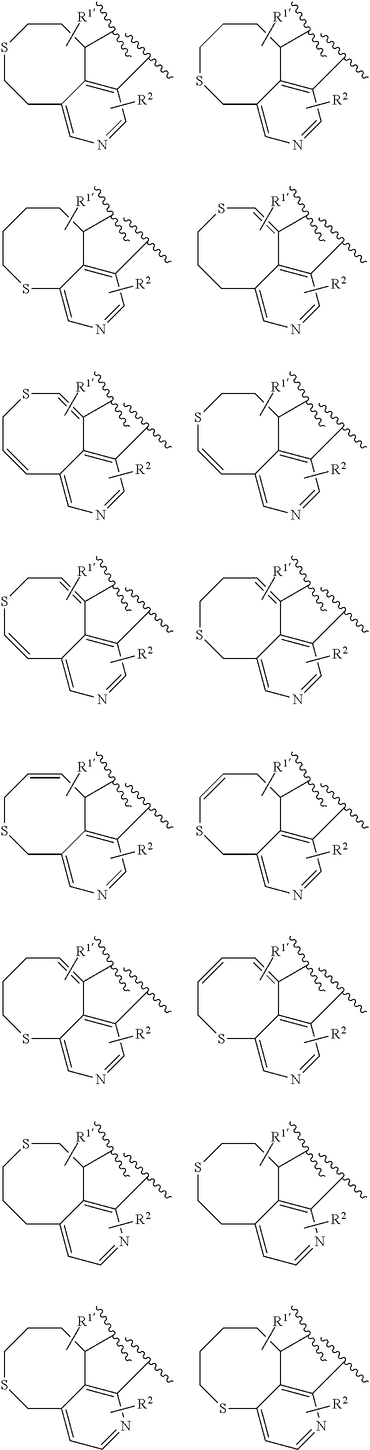

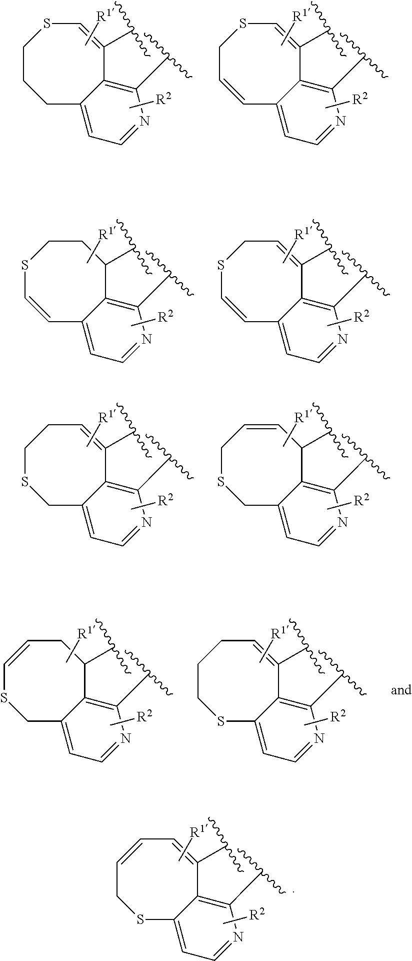

In certain embodiments

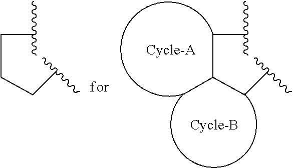

is selected from:

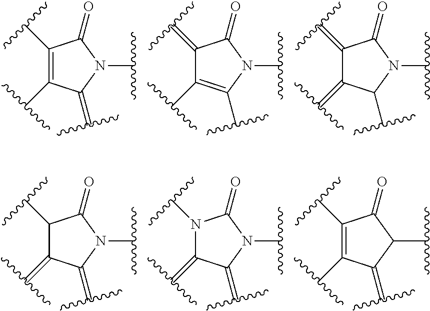 [SMILES: *C1=C(*)C(=O)N(*)C1=* ; *C1=C(*)N(*)C(=O)C1=* ; *C1C(=*)C(=*)C(=O)N1* ; *C1C(=*)C(=*)N(*)C1=O ; *n1c(=*)c(=*)n(*)c1=O ; *C1=C(*)C(=O)C(*)C1=*]

In certain embodiments

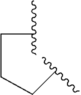 [SMILES: *CCC*]

within

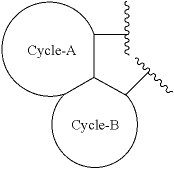 [SMILES: *CC(C)C*]

is selected from:

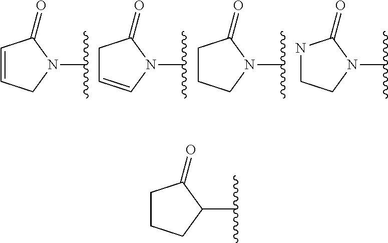

for example, when

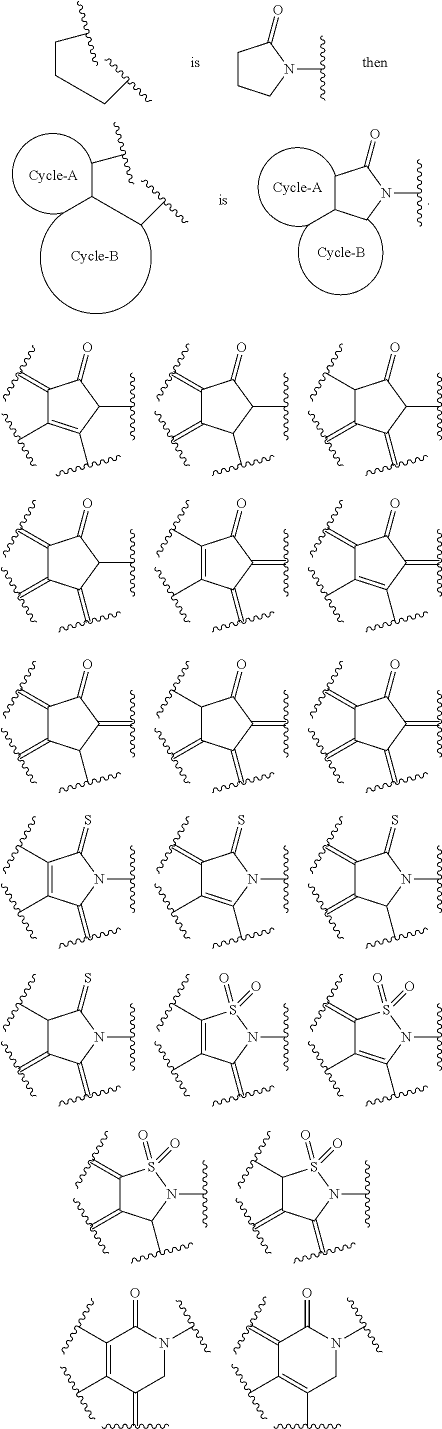

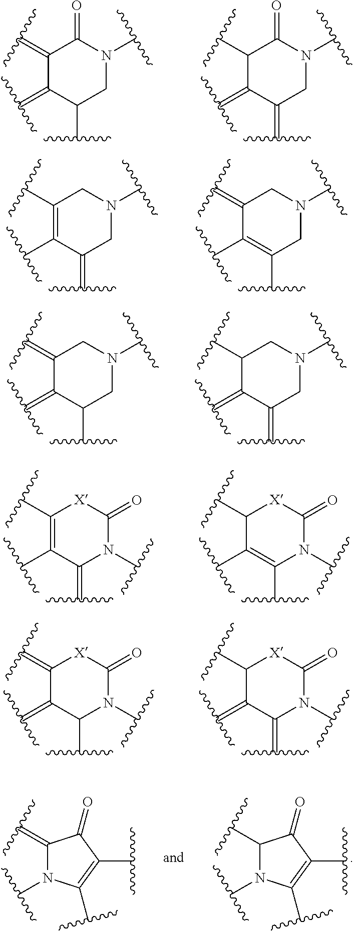

In certain embodiments

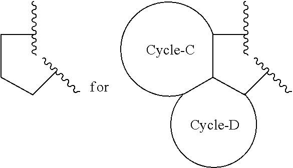

is selected from:

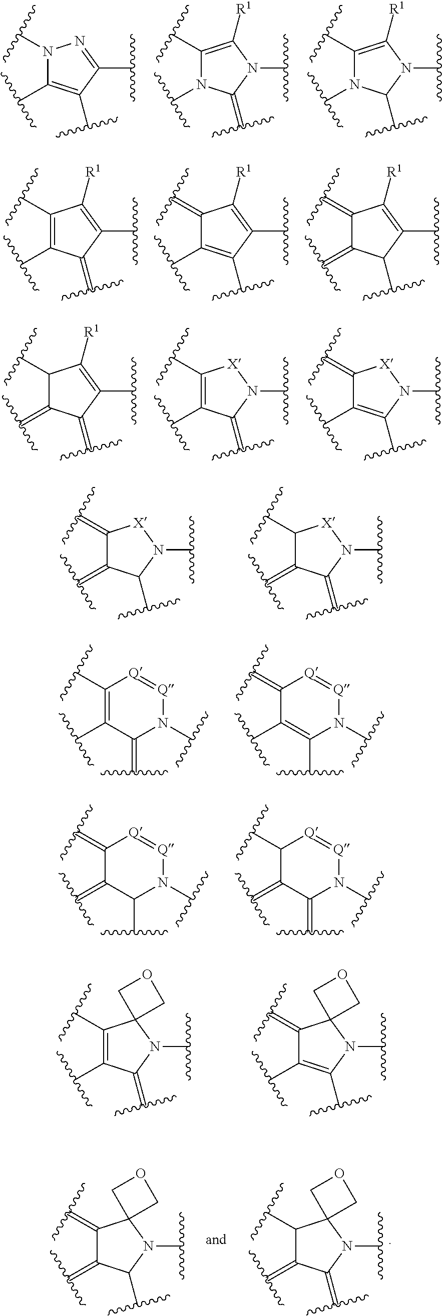

In the structures herein the structure

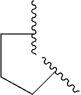 [SMILES: *CCC*]

refers to the cycloalkyl, cycloalkene, heterocyclic, aryl, or heteroaryl ring fused to either Cycle-A and Cycle-B or Cycle-C and Cycle-D.

### Embodiments of “Alkyl”

In certain embodiments “alkyl” is a C₁-C₁₀alkyl, C₁-C₉alkyl, C₁-C₈alkyl, C₁-C₇alkyl, C₁-C₆alkyl, C₁-C₅alkyl, C₁-C₄alkyl, C₁-C₃alkyl, or C₁-C₂alkyl.

In certain embodiments “alkyl” has one carbon.

In certain embodiments “alkyl” has two carbons.

In certain embodiments “alkyl” has three carbons.

In certain embodiments “alkyl” has four carbons.

In certain embodiments “alkyl” has five carbons.

In certain embodiments “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

### Embodiments of “Haloalkyl”

In certain embodiments “haloalkyl” is a C₁-C₁₀haloalkyl, C₁-C₉haloalkyl, C₁-C₂haloalkyl, C₁-C₇haloalkyl, C₁-C₆haloalkyl, C₁-C₅haloalkyl, C₁-C₄haloalkyl, C₁-C₃haloalkyl, and C₁-C₂haloalkyl.

In certain embodiments “haloalkyl” has one carbon.

In certain embodiments “haloalkyl” has one carbon and one halogen.

In certain embodiments “haloalkyl” has one carbon and two halogens.

In certain embodiments “haloalkyl” has one carbon and three halogens.

In certain embodiments “haloalkyl” has two carbons.

In certain embodiments “haloalkyl” has three carbons.

In certain embodiments “haloalkyl” has four carbons.

In certain embodiments “haloalkyl” has five carbons.

In certain embodiments “haloalkyl” has six carbons.

Non-limiting examples of “haloalkyl” include:

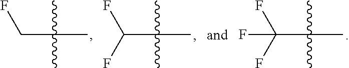

Additional non-limiting examples of “haloalkyl” include:

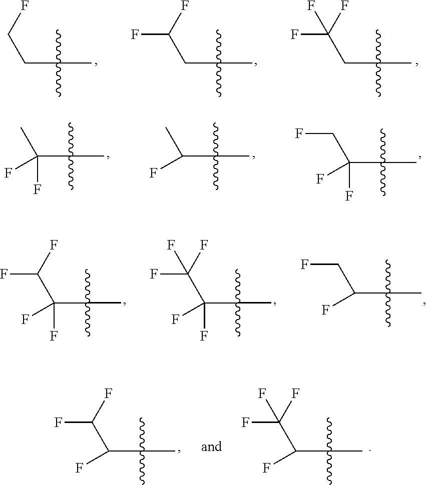

Additional non-limiting examples of “haloalkyl” include:

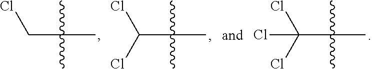

Additional non-limiting examples of “haloalkyl” include:

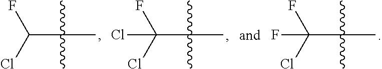

### Embodiments of “Aryl”

In certain embodiments “aryl” is a 6 carbon aromatic group (phenyl)

In certain embodiments “aryl” is a 10 carbon aromatic group (napthyl)

In certain embodiments “aryl” is a 6 carbon aromatic group fused to a heterocycle wherein the point of attachment is the aryl ring. Non-limiting examples of “aryl” include indoline, tetrahydroquinoline, tetrahydroisoquinoline, and dihydrobenzofuran wherein the point of attachment for each group is on the aromatic ring.

For example,

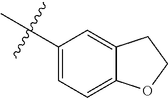 [SMILES: *c1ccc2c(c1)CCO2]

is an “aryl” group.

However,

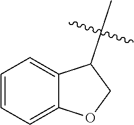 [SMILES: *C1COc2ccccc21]

is a “heterocycle” group.

In certain embodiments “aryl” is a 6 carbon aromatic group fused to a cycloalkyl wherein the point of attachment is the aryl ring. Non-limiting examples of “aryl” include dihydro-indene and tetrahydronaphthalene wherein the point of attachment for each group is on the aromatic ring.

For example,

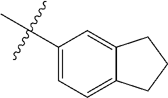 [SMILES: *c1ccc2c(c1)CCC2]

is an “aryl” group.

However,

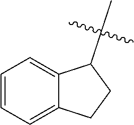 [SMILES: *C1CCc2ccccc21]

is a “cycloalkyl” group.

### Embodiments of “Heteroaryl”

In certain embodiments “heteroaryl” is a 5 membered aromatic group containing 1, 2, 3, or 4 nitrogen atoms.

Non-limiting examples of 5 membered “heteroaryl” groups include pyrrole, furan, thiophene, pyrazole, imidazole, triazole, tetrazole, isoxazole, oxazole, oxadiazole, oxatriazole, isothiazole, thiazole, thiadiazole, and thiatriazole.

Additional non-limiting examples of 5 membered “heteroaryl” groups include:

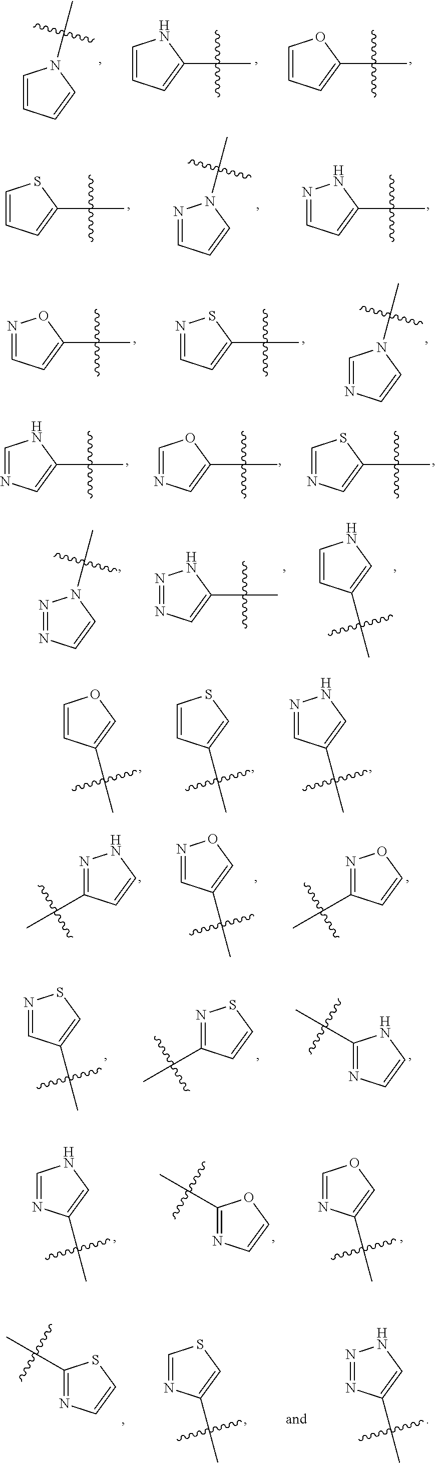

In certain embodiments “heteroaryl” is a 6 membered aromatic group containing 1, 2, or 3 nitrogen atoms (i.e. pyridinyl, pyridazinyl, triazinyl, pyrimidinyl, and pyrazinyl).

Non-limiting examples of 6 membered “heteroaryl” groups with 1 or 2 nitrogen atoms include:

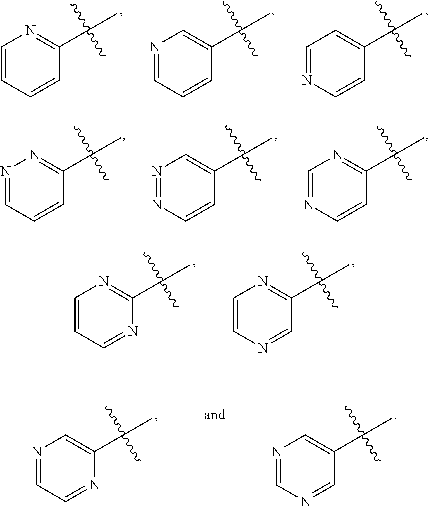

In certain embodiments “heteroaryl” is a 9 membered bicyclic aromatic group containing 1 or 2 atoms selected from nitrogen, oxygen, and sulfur.

Non-limiting examples of “heteroaryl” groups that are bicyclic include indole, benzofuran, isoindole, indazole, benzimidazole, azaindole, azaindazole, purine, isobenzofuran, benzothiophene, benzoisoxazole, benzoisothiazole, benzooxazole, and benzothiazole.

Additional non-limiting examples of “heteroaryl” groups that are bicyclic include:

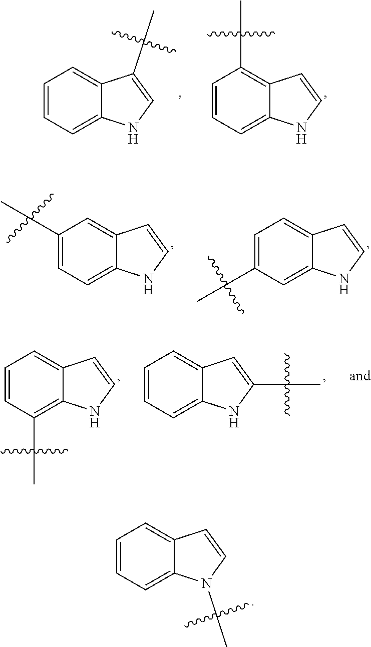

Additional non-limiting examples of “heteroaryl” groups that are bicyclic include:

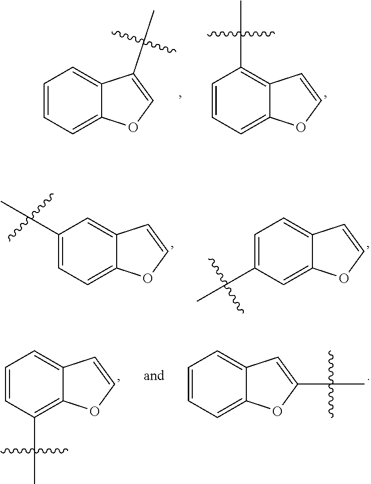 [SMILES: *c1coc2ccccc12 ; *c1cccc2occc12 ; *c1ccc2occc2c1 ; *c1ccc2ccoc2c1 ; *c1cccc2ccoc12 ; *c1cc2ccccc2o1]

Additional non-limiting examples of “heteroaryl” groups that are bicyclic include:

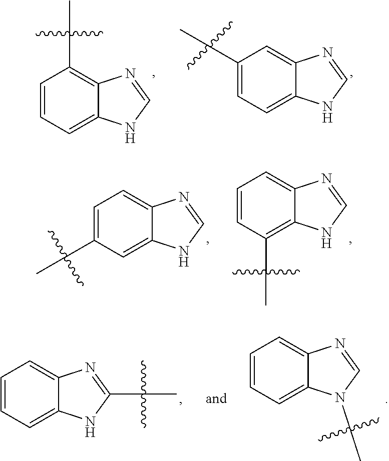

In certain embodiments “heteroaryl” is a 10 membered bicyclic aromatic group containing 1 or 2 atoms selected from nitrogen, oxygen, and sulfur.

Non-limiting examples of “heteroaryl” groups that are bicyclic include quinoline, isoquinoline, quinoxaline, phthalazine, quinazoline, cinnoline, and naphthyridine.

Additional non-limiting examples of “heteroaryl” groups that are bicyclic include:

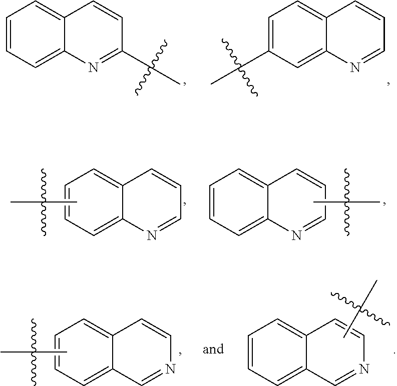

### Embodiments of “Cycloalkyl”

In certain embodiments “cycloalkyl” is a C₃-C₈cycloalkyl, C₃-C₇cycloalkyl, C₃-C₆cycloalkyl, C₃-C₅cycloalkyl, C₃-C₄cycloalkyl, C₄-C₈cycloalkyl, C₅-C₈cycloalkyl, or C₆-C₈cycloalkyl.

In certain embodiments “cycloalkyl” has three carbons.

In certain embodiments “cycloalkyl” has four carbons.

In certain embodiments “cycloalkyl” has five carbons.

In certain embodiments “cycloalkyl” has six carbons.

In certain embodiments “cycloalkyl” has seven carbons.

In certain embodiments “cycloalkyl” has eight carbons.

In certain embodiments “cycloalkyl” has nine carbons.

In certain embodiments “cycloalkyl” has ten carbons.

Non-limiting examples of “cycloalkyl” include: cyclopropyl, cyclobutyl, cyclopentyl, cyclohexyl, cycloheptyl, cyclooctyl, and cyclodecyl.

Additional non-limiting examples of “cycloalkyl” include dihydro-indene and tetrahydronaphthalene wherein the point of attachment for each group is on the cycloalkyl ring.

For example

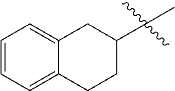 [SMILES: *C1CCc2ccccc2C1]

is an “cycloalkyl” group.

However,

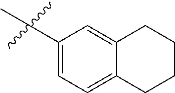 [SMILES: *c1ccc2c(c1)CCCC2]

is an “aryl” group.

Additional examples of “cycloalkyl” groups include

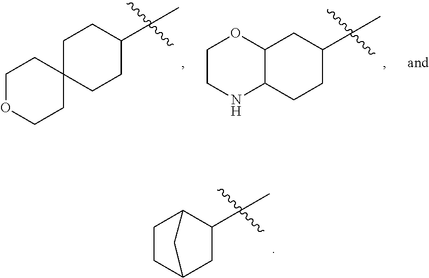 [SMILES: *C1CCC2(CCOCC2)CC1 ; *C1CCC2NCCOC2C1 ; *C1CC2CCC1C2]

### Embodiments of “Heterocycle”

In certain embodiments “heterocycle” refers to a cyclic ring with one nitrogen and 3, 4, 5, 6, 7, or 8 carbon atoms.

In certain embodiments “heterocycle” refers to a cyclic ring with one nitrogen and one oxygen and 3, 4, 5, 6, 7, or 8 carbon atoms.

In certain embodiments “heterocycle” refers to a cyclic ring with two nitrogens and 3, 4, 5, 6, 7, or 8 carbon atoms.

In certain embodiments “heterocycle” refers to a cyclic ring with one oxygen and 3, 4, 5, 6, 7, or 8 carbon atoms.

In certain embodiments “heterocycle” refers to a cyclic ring with one sulfur and 3, 4, 5, 6, 7, or 8 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of “heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include indoline, tetrahydroquinoline, tetrahydroisoquinoline, and dihydrobenzofuran wherein the point of attachment for each group is on the heterocyclic ring.

For example,

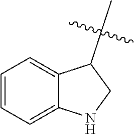 [SMILES: *C1CNc2ccccc21]

is a “heterocycle” group.

However,

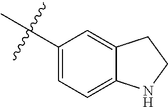 [SMILES: *c1ccc2c(c1)CCN2]

is an “aryl” group.

Non-limiting examples of “heterocycle” also include:

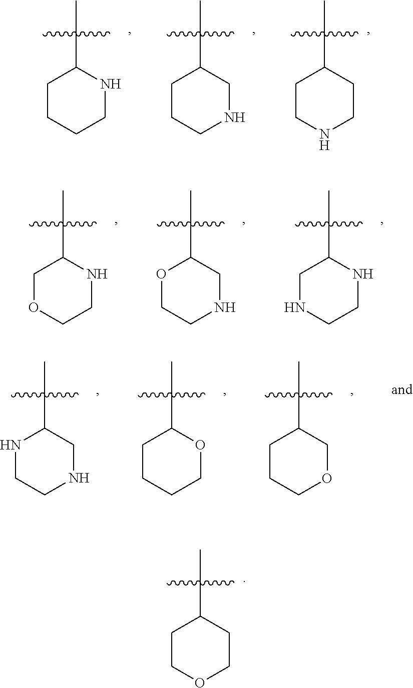

Additional non-limiting examples of “heterocycle” include:

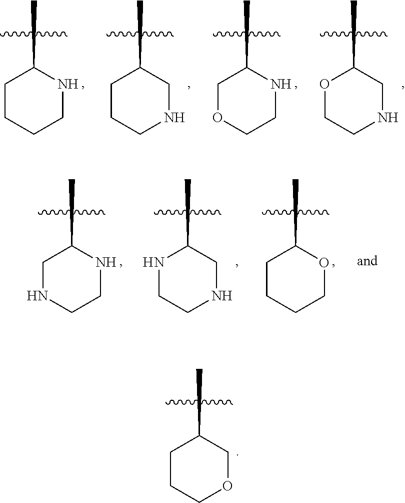

Additional non-limiting examples of “heterocycle” include:

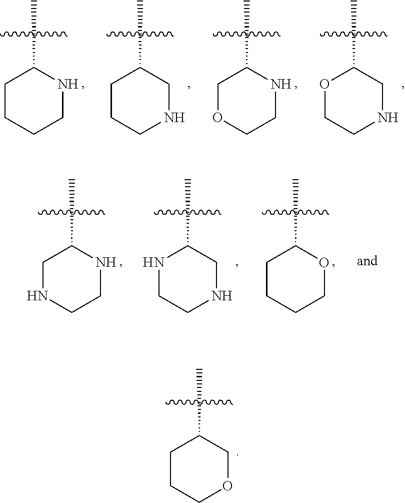

Non-limiting examples of “heterocycle” also include:

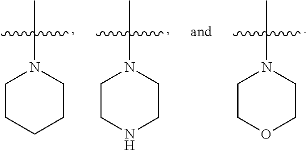

Non-limiting examples of “heterocycle” also include:

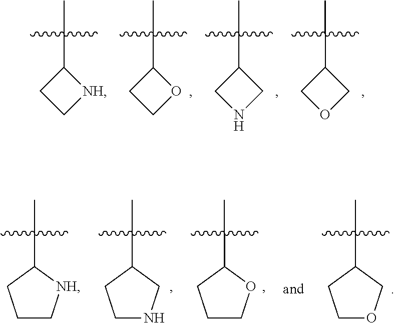

Additional non-limiting examples of “heterocycle” include:

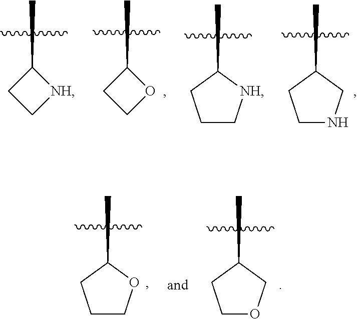

Additional non-limiting examples of “heterocycle” include:

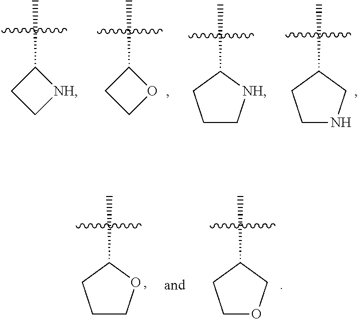

### Optional Substituents

In certain embodiments a moiety described herein that can be substituted with 1, 2, 3, or 4 substituents is substituted with one substituent.

In certain embodiments a moiety described herein that can be substituted with 1, 2, 3, or 4 substituents is substituted with two substituents.

In certain embodiments a moiety described herein that can be substituted with 1, 2, 3, or 4 substituents is substituted with three substituents.

In certain embodiments a moiety described herein that can be substituted with 1, 2, 3, or 4 substituents is substituted with four substituents.

### II. Methods of Treatment

The tricyclic compounds provided herein can bind to the cereblon receptor of CRL4^{CRBN}E3 ubiquitin ligase to create new binding sites for protein neosubstrates that are mediators of human disease, in a manner that causes the protein degradation of the neosubstrate. These compounds create a neomorphic surface that can interact directly with a target protein or target protein complex to directly or indirectly reduce protein levels. In varying embodiments, the tricyclic compounds described herein can generate a reduction in a neosubstrate target protein level via direct ubiquitination of the target protein; or ubiquitination of a neosubstrate target protein cofactor or target protein complex or other protein responsible for controlling target protein homeostasis. The compounds may cause the degradation of neosubstrate target proteins that directly bind ligand-bound cereblon; the degradation of a neosubstrate that is a cofactor that binds ligand-bound cereblon; degradation where a composite cofactor and target protein interface binds ligand-bound cereblon; the degradation of a neosubstrate target protein complex that binds ligand-bound CRBN; or the reduction of a target protein level by degradation of a protein that is not in the complex or a cofactor of the neosubstrate protein.

### A. Disease-Mediating Proteins for Degradation by Compounds of the Present Invention

It has been reported that certain proteins with a β-hairpin turn containing a glycine at a key position (a “g-loop protein” or “g-loop degron”) acts as a “structural degron” for cereblon when the cereblon is also bound to a thalidomide-like molecule (IMiD) neosubstrate protein. Such “g-loop degron” containing proteins generally include a small anti-parallel β-sheet forming a β-hairpin with an α-turn, with a geometric arrangement of three backbone hydrogen bond acceptors at the apex of a turn (positions i, i+1, and i+2), with a glycine residue at a key position (i+3) (see, e.g., Matyskiela, et al, A novel cereblon modulator recruits GSPT1 to the CRL4-CRBN ubiquitin ligase. Nature 535, 252-257 (2016); Sievers et al., Defining the human C₂H2 zinc finger degrome targeted by thalidomide analogs through CRBN. Science 362, eaat0572 (2018)). These g-loop degrons have been identified in a number of proteins, including, but not limited to, Sal-like 4 (SALL4), GSPT1, IKFZ1, IKFZ3, and CK1α, ZFP91, ZNF93, etc.

In some embodiments, a tricyclic compound of the present invention or pharmaceutical salt thereof, optionally in a pharmaceutical composition as described herein can be administered in an effective amount to a host to degrade a protein containing a g-loop degron, wherein the protein is selected from a protein kinase, C2H2 containing zinc finger protein, an RNA-recognition motif containing protein, a zinc beta ribbon containing protein, a beta-propeller containing protein, a P-loop NTPase containing protein, a really interesting new gene (RING)-finger domain containing protein, an SRC Homology 3 (SH3)-domain containing protein, an immunoglobulin E-set domain containing protein, a Tudor-domain containing protein, a zinc finger FYVE/PHD-type containing protein, an Ig-like domain containing protein, a ubiquitin-like domain containing protein, a concanavalin-like domain containing protein, a C1-domain containing protein, a Pleckstrin homology (PH)-domain containing protein, an GB-fold-domain containing protein, an NADP Rossman-fold-domain containing protein, an Actin-like ATPase domain containing protein, and a helix-turn-helix (HTH)-domain containing protein. In some embodiments, the protein kinase, C2H2 containing zinc finger protein, an RNA-recognition motif containing protein, a zinc beta ribbon containing protein, a beta-propeller containing protein, a P-loop NTPase containing protein, a really interesting new gene (RING)-finger domain containing protein, an SRC Homology 3 (SH3)-domain containing protein, an immunoglobulin E-set domain containing protein, a Tudor-domain containing protein, a zinc finger FYVE/PHD-type containing protein, an Ig-like domain containing protein, a ubiquitin-like domain containing protein, a concanavalin-like domain containing protein, a C1-domain containing protein, a Pleckstrin homology (PH)-domain containing protein, an OB-fold-domain containing protein, an NADP Rossman-fold-domain containing protein, an Actin-like ATPase domain containing protein, or a helix-turn-helix (HTH)-domain containing protein is overexpressed or contains a gain-of-function mutation. In some embodiments, the degron is stabilized by internal hydrogen bonds from an ASX motif and a ST motif.

In some embodiments, a tricyclic heterobifunctional compound of the present invention or pharmaceutical salt thereof, optionally in a pharmaceutical composition as described herein can be administered in an effective amount to a host to degrade a protein with a “g-loop degron,” wherein the “g-loop degron” comprises a [D/N]XX[S/T]G motif, wherein D=aspartic acid, N=asparagine, X can be any amino acid residue, S=serine, T=threonine, and G=glycine. In certain embodiments, the “g-loop degron” containing protein comprises an amino acid sequence of DXXSG, wherein D=aspartic acid, X can be any amino acid residue, S=serine, and G=glycine.

In another embodiment, the “g-loop degron” containing protein comprises an amino acid sequence of NXXSG, wherein N=asparagine, X can be any amino acid residue, S=serine, and G=glycine.

In yet another embodiment, the “g-loop degron” containing protein comprises an amino acid sequence of DXXTG, wherein D=aspartic acid, X can be any amino acid residue, T=threonine, and G=glycine. In still another embodiment, “g-loop degron” containing protein comprises an amino acid sequence of NXXTG, wherein N=asparagine, X can be any amino acid residue, T=threonine, and G=glycine. In some embodiments, the “g-loop degron” containing protein comprises an amino acid sequence of CXXCG, wherein C=cysteine, X can be any amino acid residue, and G=glycine. In certain embodiments, the “g-loop degron” containing protein comprises an amino acid sequence of NXXNG, wherein N=asparagine, X can be any amino acid residue, and G=glycine.

In some embodiments, a tricyclic heterobifunctional compound of the present invention or pharmaceutical salt thereof, optionally in a pharmaceutical composition as described herein can be administered in an effective amount to a host to degrade a protein with a C₂H2 zinc-finger domain containing a “g-loop degron”. In some embodiments, the zinc-finger domain has the consensus sequence C—X—X—C-G, wherein C=cysteine, X=any amino acid, and G=glycine. In an alternative embodiment, the protein with a zinc-finger domain has the consensus sequence Q-C—X—X—C-G (SEQ ID NO: 1), wherein C=cysteine, X=any amino acid, G=glycine, and Q=glutamine. In a still further embodiment, the zinc-finger domain has the consensus sequence Q-C—X₂—C-G-X₃—F-X₅-L-X₂—H—X₃—H (SEQ ID NO: 2), wherein C=cysteine, X=any amino acid, G=glycine, Q=glutamine, F=phenylalanine, L=leucine, and H=histidine. In some embodiments, the C₂H2 zinc-finger domain containing X₂—C—X₂-CG-X₂—C—X₅ (SEQ ID NO: 3), wherein C=cysteine, X=any amino acid, and G=glycine. In some embodiments, the C₂H2 zinc-finger domain containing protein is over-expressed. In some embodiments, the expression of C₂H2 zinc-finger containing protein is associated with a disease or disorder, including, but not limited to, cancer.

For example, a compound of the present invention, or pharmaceutical salt thereof, optionally in a pharmaceutical composition as described herein is administered to a host to degrade Zinc Finger Protein, Atypical E3 Ubiquitin Ligase (ZFP91). Zinc Finger Protein, Atypical E3 Ubiquitin Ligase contains a Cys2-His2 zinc finger, and protects tumor cell survival and confers chemoresistance through forkhead box A1 (FOXA1) destabilization (see, e.g., Tang, et al. The ubiquitanse ZFP91 promotes tumor cell survival and confers chemoresistance through FOXA1 destabilization, Carcinogenesis, Col. 41(1), January 2020). Zinc Finger Protein, Atypical E3 Ubiquitin Ligase is believed to act through noncanonical NF-κB pathway regulation, and its overexpression leads to increased NF-κB signaling pathway activation has been implicated in a number of cancers, including gastric cancer, breast cancer, colon cancer, kidney cancer, ovarian cancer, pancreatic cancer, stomach cancer, prostate cancer, sarcoma, and melanoma (see, e.g., Paschke, ZFP91 zinc finger protein expression pattern in normal tissues and cancers. Oncol Lett. 2019; March; 17(3):3599-3606). In certain embodiments, a compound of the present invention, or pharmaceutical salt thereof, optionally in a pharmaceutical composition as described herein is used to degrade Zinc Finger Protein, Atypical E3 Ubiquitin Ligase for the treatment of a cancer, including but not limited to, gastric cancer, breast cancer, colon cancer, lung cancer, kidney cancer, ovarian cancer, pancreatic cancer, stomach cancer, prostate cancer, sarcoma, and melanoma. In certain embodiments, a compound of the present invention, or pharmaceutical salt thereof, optionally in a pharmaceutical composition as described herein is used to degrade Zinc Finger Protein, Atypical E3 Ubiquitin Ligase for the treatment of a sarcoma, melanoma, or gastric cancer.

In another embodiment, a compound of the present invention, or pharmaceutical salt thereof, optionally in a pharmaceutical composition as described herein is administered to a host to degrade zinc finger protein 276 (ZFP276).

In yet another embodiment, a compound of the present invention, or pharmaceutical salt thereof, optionally in a pharmaceutical composition as described herein is administered to a host to degrade Zinc finger protein 653 (ZFP653). Zinc finger protein 653 may act as a more general repressor of transcription by competition with GRIP1 and other p160 coactivators for binding to SF1 (see, e.g., Borud et al., Cloning and characterization of a novel zinc finger protein that modulates the transcriptional activity of nuclear receptors. Molec. Endocr. 17: 2303-2319, 2003).

As other examples, a compound of the present invention, or pharmaceutical salt thereof, optionally in a pharmaceutical composition as described herein is administered to a host in an effective amount to degrade Zinc finger protein 692 (ZFP692). Zinc finger protein 692, also known as AICAR response element binding protein (AREBP), contains a Cys2-His2 zinc finger, and is believed to be a key modulator of hepatic glucose production regulated by AMPK in vivo (See Shirai et al., AICAR response element binding protein (AREBP), a key modulator of hepatic glucose production regulated by AMPK in vivo. Biochem Biophys Res Commun. 2011 Oct. 22; 414(2):287-91). The overexpression of and its overexpression has been associated with the promotion of colon adenocarcinoma and metastasis by activating the PI3K/AKT pathway (see, for example, Xing et al., Zinc finger protein 692 promotes colon adenocarcinoma cell growth and metastasis by activating the PI3K/AKT pathway. Int J Oncol. 2019 May; 54(5): 1691-1703), and the development of metastasis in lung adenocarcinomas and lung carcinoma. Knockdown of Zinc finger protein 692 expression via short interfering RNA reduced cell invasion and increased apoptosis in lung carcinoma cells and suppressed lung carcinoma tumor growth in a xenograft model (see, e.g., Zhang et al., ZNF692 promotes proliferation and cell mobility in lung adenocarcinoma. Biochem Biophys Res Commun. 2017 Sep. 2; 490(4):1189-1196). Accordingly, In certain embodiments, a compound of the present invention, or pharmaceutical salt thereof, optionally in a pharmaceutical composition as described herein is used to degrade Zinc finger protein 692 for the treatment of a lung or colon cancer, including a lung adenocarcinoma or carcinoma or a colon adenocarcinoma.

A tricyclic compound of the present invention, or pharmaceutical salt thereof, optionally in a pharmaceutical composition as described herein can also administered in an effective amount to a host to degrade Zinc finger protein 827 (ZFP827). Zinc finger protein 827 is a zinc finger protein that regulates alternative lengthening of telomeres (ALT) pathway by binding nuclear receptors and recruiting the nucleosome remodeling and histone deacetylation (NURD) complex to telomeres to induce homologous recombination (see, e.g., Conomos, D., Reddel, R. R., Pickett, H. A. NuRD-ZNF827 recruitment to telomeres creates a molecular scaffold for homologous recombination. Nature Struct. Molec. Biol. 21: 760-770, 2014). Zinc finger protein 827 has been associated with ALT-associated promyelocytic leukemia (PML) nuclear bodies (APBs) and other telomeric aberrations. Accordingly, in certain embodiments, a compound of the present invention, or pharmaceutical salt thereof, optionally in a pharmaceutical composition as described herein is used to degrade ZNF827 in ALT-associated disorders, including, but not limited to ALT-positive promyelocytic leukemia, osteosarcoma, adrenal/PNS neuroblastoma, breast cancer, glioblastoma, colorectal cancer, pancreatic neuroendocrine tumor (NET), neuroendocrine tumor, colorectal cancer, liver cancer, soft tissue cancers, including leiomyosarcoma, malignant fibrous histiocytoma, liposarcoma, stomach/gastric cancer, testicular cancer, and thyroid cancer.

In other embodiments, a tricyclic compound of the present invention, or pharmaceutical salt thereof, optionally in a pharmaceutical composition as described herein is administered in an effective amount to a host to degrade E4F Transcription Factor 1 protein (E4F1). E4F Transcription Factor 1 is believed to function as a ubiquitin ligase for p53, and is a key posttranslational regulator of p53 that plays an important role in the cellular life-or-death decision controlled by p53 (see, e.g., Le Cam et al., The E4F protein is required for mitotic progression during embryonic cell cycles. Molec. Cell. Biol. 24: 6467-6475, 2004). E4F1 overexpression has been associated with the development of myeloid leukemia cells (see, e.g., Hatachi et al., E4F1 deficiency results in oxidative stress-mediated cell death of leukemic cells. J Exp Med. 2011 Jul. 4; 208(7): 1403-1417). Accordingly, in certain embodiments, a compound of the present invention, or pharmaceutical salt thereof, optionally in a pharmaceutical composition as described herein is used to degrade E4F Transcription Factor 1 for the treatment of a leukemia of myelogenous origin, including but not limited to, acute myelogenous leukemia (AML), undifferentiated AML, myeloblastic leukemia with minimal cell maturation, myeloblastic leukemia with cell maturation, promyelocytic leukemia, myelomonocytic leukemia, myelomonocytic leukemia with eosinophilia, monocytic leukemia, erythroleukemia, megakaryoblastic leukemia, chronic myelogenous leukemia (CML), juvenile myelomonocytic leukemia (JMML), chronic myelomonocytic leukemia (CMML), a myeloproliferative neoplasm, including for example, polycythemia vera (PV), essential thrombocythemia (ET), myeloid metaplasia with myelofibrosis (MMM), hypereosinophilic syndrome (HES), systemic mast cell disease (SMCD), myelofibrosis, and primary myelofibrosis. E4F1 expression is also essential for survival in p53-deficient cancer cells (see, e.g., Rodier et al., The Transcription Factor E4F1 Coordinates CHK1-Dependent Checkpoint and Mitochondrial Functions. Cell Reports Volume 11, ISSUE 2, P220-233, Apr. 14, 2015). Accordingly, in certain embodiments, a compound of the present invention, or pharmaceutical salt thereof, optionally in a pharmaceutical composition as described herein is used to degrade E4F Transcription Factor 1 for the treatment of a p53-deficient associated disorder, including, but not limited to ovarian cancer, small cell lung cancer, pancreatic cancer, head and neck squamous cell carcinoma, and triple negative breast cancer.

In another aspect, a tricyclic compound of the present invention, or pharmaceutical salt thereof, optionally in a pharmaceutical composition as described herein is administered in an effective amount to a host to degrade Zinc finger protein 517 (ZFP517). Zinc finger protein 517 has been identified as an oncogenic driver in adrenocortical carcinoma (ACC) (see, e.g., Rahane et al., Establishing a human adrenocortical carcinoma (ACC)-specific gene mutation signature. Cancer Genet. 2019; 230:1-12). Accordingly, in certain embodiments, a compound of the present invention, or pharmaceutical salt thereof, optionally in a pharmaceutical composition as described herein is used to Zinc finger protein 517 for the treatment of adrenocortical carcinoma.

In yet another aspect, a tricyclic compound of the present invention, or pharmaceutical salt thereof, optionally in a pharmaceutical composition as described herein is administered in an effective amount to a host to degrade Zinc finger protein 582 (ZFP582). Zinc finger protein 582 is believed to be involved in n DNA damage response, proliferation, cell cycle control, and neoplastic transformation, most notably cervical, esophageal, and colorectal cancer (see, e.g., Huang et al., Methylomic analysis identifies frequent DNA methylation of zinc finger protein 582 (ZNF582) in cervical neoplasms. PLoS One 7: e41060, 2012; Tang et al., Aberrant DNA methylation of PAX1, SOXI and ZNF582 genes as potential biomarkers for esophageal squamous cell carcinoma. Biomedicine & Pharmacotherapy Volume 120, December 2019, 109488; Harada et al., Analysis of DNA Methylation in Bowel Lavage Fluid for Detection of Colorectal Cancer. Cancer Prev Res; 7(10); 1002-10; 2014). Accordingly, in certain embodiments, a compound of the present invention, or pharmaceutical salt thereof, optionally in a pharmaceutical composition as described herein is used to degrade Zinc finger protein 582 for the treatment of a cancer, including but not limited to cervical cancer, including cervical adenocarcinoma, esophageal cancer, including squamous cell carcinoma and adenocarcinoma, and colorectal cancer.

In another embodiment, a tricyclic compound of the present invention, or pharmaceutical salt thereof, optionally in a pharmaceutical composition as described herein is administered in an effective amount to a host to degrade Zinc finger protein 654 (ZFP654).

Alternatively, a tricyclic compound of the present invention, or pharmaceutical salt thereof, optionally in a pharmaceutical composition as described herein is administered in an effective amount to a host to degrade Zinc finger protein 787 (ZFP787).

A tricyclic compound of the present invention, or pharmaceutical salt thereof, optionally in a pharmaceutical composition as described herein can be administered in an effective amount to a host to degrade Hypermethylated in Cancer 1 (HIC1) protein. Hypermethylated in Cancer 1 protein contains an N-terminal BTB/POZ protein-protein interaction domain and 5 Kruppel-like C₂H2 zinc finger motifs in its C-terminal half (see, e.g., Deltour et al., The carboxy-terminal end of the candidate tumor suppressor gene HIC-1 is phylogenetically conserved. Biochim. Biophys. Acta 1443: 230-232, 1998). Expression of Hypermethylated in Cancer 1 protein gene disorder Miller-Dieker syndrome (see, e.g., Grimm et al., Isolation and embryonic expression of the novel mouse gene Hic1, the homologue of HIC1, a candidate gene for the Miller-Dieker syndrome. Hum. Molec. Genet. 8: 697-710, 1999).

A tricyclic compound of the present invention, or pharmaceutical salt thereof, optionally in a pharmaceutical composition as described herein is administered in an effective amount to a host to degrade Hypermethylated in Cancer 2 (HIC2) protein.

A tricyclic compound of the present invention, or pharmaceutical salt thereof, optionally in a pharmaceutical composition as described herein can be administered in an effective amount to a host to degrade GDNF-Inducible Zinc Finger Protein 1 (GZF1). GDNF-Inducible Zinc Finger Protein 1 is a transcriptional regulator that binds to a 12-bp GZF1 response element (GRE) and represses gene transcription (see, e.g., Morinaga et al., GDNF-inducible zinc finger protein 1 is a sequence-specific transcriptional repressor that binds to the HOXA10 gene regulatory region. Nucleic Acids Res. 33: 4191-4201, 2005).

Alternatively, for example, a tricyclic compound of the present invention, or pharmaceutical salt thereof, optionally in a pharmaceutical composition as described herein can be administered in an effective amount to a host to degrade Odd Skipped Related 1 (OSR1) protein. Odd Skipped Related 1 protein contains 3 C2H2-type zinc fingers, a tyrosine phosphorylation site, and several putative PXXP SH3 binding motifs (see, e.g., Katoh, M. Molecular cloning and characterization of OSR1 on human chromosome 2p24. Int. J. Molec. Med. 10: 221-225, 2002).

In another aspect, a tricyclic compound of the present invention, or pharmaceutical salt thereof, optionally in a pharmaceutical composition as described herein is administered in an effective amount to a host to degrade Odd Skipped Related 2 (OSR2) protein.

In yet another embodiment, a selected tricyclic compound of the present invention, or pharmaceutical salt thereof, optionally in a pharmaceutical composition as described herein can be administered to a host in an effective amount to degrade SAL-Like 4 (SALL4) protein. SAL-Like 4 protein has 3 C₂H2 double zinc finger domains of the SAL-type, the second of which has a single C₂H2 zinc finger attached at its C-terminal end, as well as an N-terminal C₂HC zinc finger motif typical for vertebrate SAL-like proteins. SAL-Like 4 protein mutations are associated with the development of Duane-radial ray syndrome (see, e.g., Borozdin et al., SALL4 deletions are a common cause of Okihiro and acro-renal-ocular syndromes and confirm haploinsufficiency as the pathogenic mechanism. J. Med. Genet. 41: e113, 2004). SAL-Like 4 protein overexpression is associated with the promotion, growth and metastasis of a number of cancers, including lung cancer, gastric cancer, liver cancer, renal cancer, myelodysplastic syndrome, germ cell-sex cord-stromal tumors including dysgerminoma, yolk sac tumor, and choriocarcinoma, and leukemia, among others. Accordingly, in certain embodiments, a compound of the present invention, or pharmaceutical salt thereof, optionally in a pharmaceutical composition as described herein is used to degrade SAL-Like 4 protein for the treatment of a cancer, including but not limited to, gastric cancer, liver cancer, renal cancer, myelodysplastic syndrome, germ cell-sex cord-stromal tumors including dysgerminoma, yolk sac tumor, and choriocarcinoma, and leukemia, among others.

A selected tricyclic compound of the present invention, or pharmaceutical salt thereof, optionally in a pharmaceutical composition as described herein can also be administered in an effective amount to a host to degrade B-Cell Lymphoma 6 (BCL6) protein. B-Cell Lymphoma 6 contains an autonomous transrepressor domain, and 2 noncontiguous regions, including the POZ motif, mediate maximum transrepressive activity. Translocations of the B-Cell Lymphoma 6 gene translocations are associated with the development of myeloproliferative disorders such as non-Hodgkin lymphomas. B-Cell Lymphoma 6 overexpression prevents increase in reactive oxygen species and inhibits apoptosis induced by chemotherapeutic reagents in cancer cells (see, e.g., Tahara et al., Overexpression of B-cell lymphoma 6 alters gene expression profile in a myeloma cell line and is associated with decreased DNA damage response. Cancer Sci. 2017 August; 108(8):1556-1564; Cardenas et al., The expanding role of the BCL6 oncoprotein as a cancer therapeutic target. Clin Cancer Res. 2017 Feb. 15; 23(4): 885-893). Accordingly, in certain embodiments, a compound of the present invention, or pharmaceutical salt thereof, optionally in a pharmaceutical composition as described herein is used to degrade B-Cell Lymphoma 6 for the treatment of a cancer, including but not limited to a hematologic or solid tumor, for example, but not limited to a B-cell leukemia or lymphoma, for example, but not limited to diffuse large B-cell lymphomas (DLBCLs) and ABC-DLBCL subtypes, B-acute lymphoblastic leukemia, chronic myeloid leukemia, breast cancer and non-small cell lung cancer.

Further, a selected tricyclic compound of the present invention, or pharmaceutical salt thereof, optionally in a pharmaceutical composition as described herein is administered in an effective amount to a host to degrade B-Cell Lymphoma 6B (BCL6B) protein. B-Cell Lymphoma 6B protein contains an N-terminal POZ domain and 5 C-terminal zinc finger motifs, and is believed to act as a transcriptional repressor (see, e.g., Okabe et al., BAZF, a novel Bc16 homolog, functions as a transcriptional repressor. Molec. Cell. Biol. 18: 4235-4244, 1998). Overexpression of B-Cell Lymphoma 6B protein has been associated with the development of germ cell tumors (Ishii et al., FGF2 mediates mouse spermatogonial stem cell self-renewal via upregulation of Etv5 and Bc16b through MAP2K1 activation. Development 139, 1734-1743 (2012)). Accordingly, in certain embodiments, a compound of the present invention, or pharmaceutical salt thereof, optionally in a pharmaceutical composition as described herein is used to degrade B-Cell Lymphoma 6B for the treatment of a cancer, including but not limited to, a germ cell cancer including but not limited to germinoma, including dysgerminoma and seminoma, a teratoma, yolk sac tumor, and choriocarcinomas.

Alternatively, a selected tricyclic compound of the present invention, or pharmaceutical salt thereof, optionally in a pharmaceutical composition as described herein can be administered in an effective amount to a host to degrade Early Growth Response 1 (EGR1) protein. Early Growth Response 1 protein directly controls transforming growth factor-beta−1 gene expression, and has been shown to be involved in the proliferation and survival of prostate cancer cells by regulating several target genes, including cyclin D2 (CCND2), p19(Ink4d), and Fas, as well as glioma cells (see, e.g., Virolle et al., Erg1 promotes growth and survival of prostate cancer cells: identification of novel Egr1 target genes. J. Biol. Chem. 278: 11802-11810, 2003; Chen et al., Inhibition of EGR1 inhibits glioma proliferation by targeting CCND1 promoter. Journal of Experimental & Clinical Cancer Research Volume 36, Article number: 186 (2017)). One mechanism used by Egr1 to confer resistance to apoptotic signals was the ability of Egr1 to inhibit Fas expression, leading to insensitivity to FasL. Accordingly, in certain embodiments, a compound of the present invention, or pharmaceutical salt thereof, optionally in a pharmaceutical composition as described herein is used to degrade Early Growth Response 1 protein for the treatment of a cancer, including but not limited to a prostate cancer or glioma including, but not limited to, pilocytic astrocytoma, diffuse astrocytoma, anaplastic astrocytoma, glioblastoma multiforme.

In yet another aspect, a selected tricyclic compound of the present invention, or pharmaceutical salt thereof, optionally in a pharmaceutical composition as described herein can be administered in an effective amount to a host to degrade Early Growth Response 4 (EGR4) protein. Early Growth Response 4 protein contains 3 zinc fingers of the C₂/H2 subtype near the carboxy terminus (see, e.g., Crosby et al., Neural-specific expression, genomic structure, and chromosomal localization of the gene encoding the zinc-finger transcription factor NGFI-C. Proc. Nat. Acad. Sci. 89: 4739-4743, 1992). Overexpression of Early Growth Response 4 protein has been associated with the development of cholangiocarcinoma (see, e.g., Gong et al., Gramicidin inhibits cholangiocarcinoma cell growth by suppressing EGR4. Artificial Cells, Nanomedicine, and Biotechnology, 48:1, 53-59 (2019)). Accordingly, in certain embodiments, a compound of the present invention, or pharmaceutical salt thereof, optionally in a pharmaceutical composition as described herein is used to degrade Early Growth Response 4 protein for the treatment of a cancer, including but not limited to cholangiocarcinoma.

In certain aspects, a selected tricyclic compound of the present invention, or pharmaceutical salt thereof, optionally in a pharmaceutical composition as described herein can be administered in an effective amount to a host to degrade Sal-Like 1 (SALL1) protein.

In an alternative embodiment, a selected tricyclic compound of the present invention, or pharmaceutical salt thereof, optionally in a pharmaceutical composition as described herein can be administered in an effective amount to a host to degrade Sal-Like 3 (SALL3) protein. The SALL3 protein contains 4 double zinc finger (DZF) domains, each of which contains sequences identical or closely related to the SAL box, a characteristic stretch of 8 amino acids within the second zinc finger motif.

In yet another embodiment, a selected tricyclic compound of the present invention, or pharmaceutical salt thereof, optionally in a pharmaceutical composition as described herein can be administered in an effective amount to a host to degrade Tumor protein p63 (TP63). Tumor protein p63 overexpression has been associated with lung cancer development and poor prognosis, radiation resistance in oral cancers and head and neck cancers, squamous cell carcinoma of the skin (see, e.g., Massion et al., Significance of p63 amplification and overexpression in lung cancer development and prognosis. Cancer Res. 2003 Nov. 1; 63(21):7113-21; Moergel et al., Overexpression of p63 is associated with radiation resistance and prognosis in oral squamous cell carcinoma. Oral Oncol. 2010 September; 46(9):667-71). Accordingly, in certain embodiments, a compound of the present invention, or pharmaceutical salt thereof, optionally in a pharmaceutical composition as described herein is used to degrade Tumor protein p63 for the treatment of a cancer, including but not limited to non-small cell lung cancer, small cell lung cancer, head and neck cancer, and squamous cell carcinoma of the skin.

In yet another embodiment, a selected tricyclic compound of the present invention, or pharmaceutical salt thereof, optionally in a pharmaceutical composition as described herein can be administered in an effective amount to a host to degrade Widely-Interspaced Zinc Finger-Containing (WIZ) Protein.

A selected tricyclic compound of the present invention, or pharmaceutical salt thereof, optionally in a pharmaceutical composition as described herein can also be administered in an effective amount to a host to degrade Zinc Finger and BTB Domain Containing Protein 7A (ZBTB7A). Zinc Finger and BTB Domain Containing Protein 7A expression is associated with a number of cancers, including prostate cancer, non-small cell lung cancer, bladder, breast cancer, prostate, ovarian, oral squamous cell carcinoma, and hepatocellular carcinoma (see, e.g., Han et al., ZBTB7A Mediates the Transcriptional Repression Activity of the Androgen Receptor in Prostate Cancer. Cancer Res 2019; 79:5260-71; Molloy et al., ZBTB7A governs estrogen receptor alpha expression in breast cancer. Journal of Molecular Cell Biology, Volume 10, Issue 4, August 2018, Pages 273-284). Accordingly, in certain embodiments, a compound of the present invention, or pharmaceutical salt thereof, optionally in a pharmaceutical composition as described herein is used to degrade Zinc Finger and BTB Domain Containing Protein 7A for the treatment of a cancer, including but not limited to prostate cancer, non-small cell lung cancer, breast cancer, oral squamous cell carcinoma, prostate, ovarian, glioma, bladder, and hepatocellular carcinoma.

In other aspects, a selected tricyclic compound of the present invention, or pharmaceutical salt thereof, optionally in a pharmaceutical composition as described herein can be administered in an effective amount to a host to degrade Zinc Finger and BTB Domain Containing Protein 7B (ZBTB7B). Zinc Finger and BTB Domain Containing Protein 7B expression has been associated with breast, prostate, urothelial, cervical, and colorectal cancers. Accordingly, in certain embodiments, a compound of the present invention, or pharmaceutical salt thereof, optionally in a pharmaceutical composition as described herein is used to degrade Zinc Finger and BTB Domain Containing Protein 7B for the treatment of a cancer, including but not limited to breast, prostate, urothelial, cervical, and colorectal cancers.

A selected tricyclic compound of the present invention, or pharmaceutical salt thereof, optionally in a pharmaceutical composition as described herein can be administered in an effective amount to a host to degrade casein kinase I, alpha I (CK1α or CK1-alpha). CK1-alpha is a bifunctional regulator of NF-kappa-B (see, e.g., Bidere et al., Casein kinase 1-alpha governs antigen-receptor-induced NF-kappa-B activation and human lymphoma cell survival. Nature 458: 92-96, 2009). CK1-alpha dynamically associates with the CBM complex on T cell receptor engagement to participate in cytokine production and lymphocyte proliferation. However, CK1-alpha kinase activity has a contrasting role by subsequently promoting the phosphorylation and inactivation of CARMA1. CK1-alpha has thus a dual ‘gating’ function which first promotes and then terminates receptor-induced NF-kappa-B. ABC DLBCL cells required CK1-alpha for constitutive NF-kappa-B activity, indicating that CK1-alpha functions as a conditionally essential malignancy gene. Expression of CK1-alpha has been associated with myelodysplastic disease with depletion of 5q (del(5q) MDS (see, e.g., Kronke, et al., Lenalidomide induces ubiquitination and degradation of CK1-alpha in del(5q) MDS. Nature 523: 183-188, 2015), colorectal cancer, breast cancer, leukemia, multiple myeloma, lung cancer, diffuse large B cell lymphoma, non-small cell lung cancer, and pancreatic cancer, amongst others (see, e.g., Richter et al., CK1α overexpression correlates with poor survival in colorectal cancer. BMC Cancer. 2018; 18: 140; Jiang et al., Casein kinase 1α: biological mechanisms and theranostic potential. Cell Commun Signal. 2018; 16: 23). Accordingly, in some embodiments, a compound of the present invention, or pharmaceutical salt thereof, optionally in a pharmaceutical composition as described herein is used to degrade casein kinase I, alpha I for the treatment of a cancer, including but not limited to colorectal cancer, breast cancer, leukemia, multiple myeloma, lung cancer, diffuse large B cell lymphoma, non-small cell lung cancer, pancreatic cancer, myelodysplastic syndromes including but not limited to 5q-syndrome, refractory cytopenia with unilineage dysplasia, refractory anemia, refractory neutropenia, and refractory thrombocytopenia, refractory anemia with ring sideroblasts, refractory cytopenia with multilineage dysplasia (RCMD), refractory anemias with excess blasts (REAB) I and II, refractory anemia with excess blasts in transformation (RAEB-T), chronic myelomonocytic leukemia (CMML), myelodysplasia unclassifiable, refractory cytopenia of childhood (dysplasia in childhood).

A selected tricyclic compound of the present invention, or pharmaceutical salt thereof, optionally in a pharmaceutical composition as described herein can also be administered inan effective amount to a host to degrade Family with Sequence Similarity 83, Member H (FAM83H). FAM83H is believed to be involved in the progression of human cancers in conjunction with tumor-associated molecules, such as MYC and β-catenin, and overexpression has been associated with lung, breast, colon, liver, ovary, pancreas, prostate, oesophageal, glioma, hepatocellular carcinoma, thyroid, renal cell carcinoma, osteosarcoma, and stomach cancers (see, e.g., Kim et al., FAM83H is involved in stabilization of β-catenin and progression of osteosarcomas. Journal of Experimental & Clinical Cancer Research volume 38, Article number: 267 (2019)). Accordingly, in some embodiments, a compound of the present invention, or pharmaceutical salt thereof, optionally in a pharmaceutical composition as described herein is used to degrade FAM83H for the treatment of a cancer, including but not limited to, lung, breast, colon, liver, ovary, pancreas, prostate, oesophageal, glioma, thyroid, liver cancer, including but not limited to hepatocellular carcinoma, renal cell carcinoma, osteosarcoma, and stomach cancers.

Alternatively, a selected tricyclic compound of the present invention, or pharmaceutical salt thereof, optionally in a pharmaceutical composition as described herein can be administered in an effective amount to a host to degrade Zinc-finger and BTB domain containing protein 16 (ZBTB16). Overexpression and translocation of ZBTB16 has been associated with the development of various hematological cancers, including acute promyelocytic leukemia (see, e.g., Zhang et al., Genomic sequence, structural organization, molecular evolution, and aberrant rearrangement of promyelocytic leukemia zinc finger gene. Proc. Nat. Acad. Sci. 96: 11422-11427, 1999). Accordingly, in some embodiments, a compound of the present invention, or pharmaceutical salt thereof, optionally in a pharmaceutical composition as described herein is used to degrade ZBTB16 for the treatment of a cancer, including but not limited to a hematological cancer including but not limited to a leukemia or lymphoma, including but not limited to acute promyelocytic leukemia, acute lymphoblastic leukemia, Adult T-cell lymphoma/ATL, and Burkitt's lymphoma.

In an alternative embodiment, a selected tricyclic compound of the present invention, or pharmaceutical salt thereof, optionally in a pharmaceutical composition as described herein can be administered in an effective amount to a host to degrade AT-Rich Interaction Domain-Containing Protein 2 (ARID2). ARID2 is a subunit of the PBAF chromatin-remodeling complex, which facilitates ligand-dependent transcriptional activation by nuclear receptors (see, e.g., Yan et al., PBAF chromatin-remodeling complex requires a novel specificity subunit, BAF200, to regulate expression of selective interferon-responsive genes. Genes Dev. 19: 1662-1667, 2005).

In another aspect, a selected tricyclic compound of the present invention, or pharmaceutical salt thereof, optionally in a pharmaceutical composition as described herein can be administered in an effective amount to a host to degrade Polybromo associated BAF (PBAF). Mutations in PBAF have been associated with the development of synovial sarcomas and multiple myeloma (see, e.g., Alfert et al., The BAF complex in development and disease. Epigenetics & Chromatin volume 12, Article number: 19 (2019)). Accordingly, in some embodiments, a compound of the present invention, or pharmaceutical salt thereof, optionally in a pharmaceutical composition as described herein is used to degrade PBAF for the treatment of a cancer, including but not limited to synovial sarcoma and multiple myeloma.

In other embodiments, the selected tricyclic compound of the present invention when administered after binding to and forming a neomorphic surface with cereblon, is capable of binding a number of neosubstrates resulting in a form of “poly-pharmacology.” For example, the tricyclic compound may bind and degrade IRAK4, IKZF1 and/or 3, and or Aiolos. In other examples, the tricyclic compound, when administered, is able to degrade two or more of the proteins named above or herein, for example, SALL4 and IKZF ⅓ or IKZF 2/4.

In certain specific embodiments the compound of the present invention degrades an IKZF protein. The Ikaros (“IKZF”) family is a series of zinc-finger protein transcription factors that are important for certain physiological processes, particularly lymphocyte development (see Fan, Y. and Lu, D. Acta Pharmaceutica Sinica B, 2016, 6:513-521). Ikaros (“IKZF1”) was first discovered in 1992 (see Georgopoulos, K. et al. Science, 1992, 258:802-812), and over the subsequent two decades four additional homologs have been identified: Helios (“IKZF2”), Aiolos (“IKZF3”), Eos (“IKZF4”), and Pegasus (“IKZF5”) (see John, L. B., and Ward, A. C. Mol Immunol, 2011, 48:1272-1278). Each homolog gene can produce several protein isoforms through alternative splicing, theoretically allowing for the generation of a large number of protein complexes through different combinations of the various homologs.

The distribution of various members of the Ikaros protein family within the body varies significantly. Ikaros, Helios, and Aiolos are mainly present in lymphoid cells and their corresponding progenitors, with Ikaros additionally also detected in the brain, and Ikaros and Helios also detected in erythroid cells. Eos and Pegasus are more widely spread, and found in skeletal muscle, the liver, the brain, and the heart (see Perdomo, J. et al. J Biol Chem, 2000, 275:38347-38354; Schmitt, C. et al. Apoptosis, 2002, 7:277-284; Yoshida, T. and Georgopoulos, K. Int J Hematol, 2014, 100:220-229).

In certain embodiments a compound of the present invention pharmaceutical salt thereof, optionally in a pharmaceutical composition as described herein is used to degrade Ikaros or Aiolos, which is a mediator of the disorder affecting the patient, such as a human. The control of protein level afforded by any of the compounds of the present invention provides treatment of a disease state or condition, which is modulated through Ikaros or Aiolos by lowering the level of that protein in the cell, e.g., cell of a patient, or by lowering the level of downstream proteins in the cell.

In certain embodiments a compound of the present invention can provide a therapeutic effect by direct degradation of Ikaros or Aiolos which may change the transcriptional regulation of a protein downstream of Ikaros or Aiolos.

Translation termination factor G1 to S phase transition protein 1 (GSPT1) is a termination factor that is essential for the G1 to S phase transition of the cell cycle and is known to function as a polypeptide chain release factor 3 (eRF3) (Kikuchi et al., 1988). GSPT1 interacts with eRF1 to mediate stop codon recognition and nascent protein release from ribosomes (Cheng et al. Genes Dev., 2009, 23, 1106-1118).

Studies have shown that GSPT1 is involved in processes such as cell cycle, apoptosis and transcription (Hegde et al. J. Biol. Chem. 2003; Park et al. Oncogene, 2008, 27, 1297), and therefore GSPT1 may play a role in abnormal cellular proliferation. For example, overexpression of eRF3/GSTP1 in certain intestinal type gastric tumors was related to an increase in the translation efficiency of specific oncogenic transcripts (Malta-Vacas et al. J. Clin. Pathol. 2005, 58, 621).

In certain embodiments a compound of the present invention pharmaceutical salt thereof, optionally in a pharmaceutical composition as described herein is used to degrade GSTP1, which is a mediator of the disorder affecting the patient, such as a human. The control of protein level afforded by any of the compounds of the present invention provides treatment of a disease state or condition, which is modulated through GSTP1 by lowering the level of that protein in the cell, e.g., cell of a patient, or by lowering the level of downstream proteins in the cell.

In certain embodiments a compound of the present invention can provide a therapeutic effect by direct degradation of GSTP1 which may change the transcriptional regulation of a protein downstream of GSTP1.

In some embodiments, a coronavirus protein is degraded. In some embodiments, the coronavirus protein is a beta coronavirus protein. In some embodiments, the coronavirus protein is a Severe Acute Respiratory Syndrome (SARS)-CoV protein, a Middle Eastern Respiratory Syndrome (MERS)-CoV protein, or a SARS-CoV-2 protein. In some embodiments, the Target Protein is a SARS-CoV-2 protein. In some embodiments, the SARS-CoV2 protein is selected from a structural protein selected from a spike (S) protein (Accession #BCA87361.1), a membrane (M) protein (Accession #BCA87364.1), an envelope (E) protein (Accession #BCA87363.1), or a nucleocapsid phosphoprotein (N) protein (Accession #BCA87368.1), or a sequence at least 70%, 75%, 80%, 85%, 90%, 95%, or 98% homologous thereto, or a homolog, mutant, conjugate, derivative, fragment, or ortholog thereof. In some embodiments, the SARS-CoV2 protein is a non-structural protein, including nsp1 (leader protein) (Accession #YP_009725297.1), nsp2 (Accession #YP_009725298.1), nsp3 (papain-like proteinase) (Accession #YP_009725299.1), nsp4 (Accession #YP_009725300.1), nsp5 (3C-like proteinase) (Accession #YP_009725301.1), nsp6 (putative transmembrane domain) (Accession #YP_009725302.1), nsp7 (Accession #YP_009725303.1), nsp8 (primase) (Accession #YP_009725304.1), nsp9 (Accession #YP_009725305.1), nsp10 (Accession #YP_009725306.1), nsp11 (Accession #YP_009725312.1), nsp12 (RNA dependent RNA polymerase) (Accession #YP_009725307.1), nsp13 (helicase) (Accession #YP_009725308.1), nsp14 (3′-5′ exonuclease, guanine N7-methyltransferase) (Accession #YP_009725309.1), nsp15 (endoRNAse) (Accession #YP_009725310.1), or nsp16 (2′-O-ribose-methyltransferase) (Accession #YP_009725311.1), or a sequence at least 70%, 75%, 80%, 85%, 90%, 95%, or 98% homologous thereto, or a homolog, mutant, conjugate, derivative, fragment, or ortholog thereof. In some embodiments, the SARS-CoV2 protein is selected from ORF3a protein (Accession #BCA87362.1), ORF6 protein (accessory protein 6) (Accession #BCA87365.1), ORF7a protein (accessory protein 7a)(Accession #BCA87366.1), ORF7b protein (accessory protein 7b) (Accession #BCB15096.1), ORF8 protein (Accession #QJA17759.1), ORF9b protein (accessory protein 9b) (UniprotKB-PODTD2), or ORF10 protein (Accession #BCA87369.1), or a sequence at least 70%, 75%, 80%, 85%, 90%, 95%, or 98% homologous thereto, or a homolog, mutant, conjugate, derivative, fragment, or ortholog thereof. In some embodiments, the SARS-CoV2 protein is ORF3b protein (see Konno et al., SARS-CoV-2 ORF3b Is a Potent Interferon Antagonist Whose Activity Is Increased by a Naturally Occurring Elongation Variant. Cell Reports, Volume 32, Issue 12, 22 Sep. 2020, 108185) encoded by nucleotides 25814-25880 of NCBI Reference Sequence: NC_045512.2, or a sequence at least 70%, 75%, 80%, 85%, 90%, 95%, or 98% homologous thereto, or a homolog, mutant, conjugate, derivative, fragment, or ortholog thereof.

In other embodiments, the protein that is degraded is a viral protein of a virus other than coronavirus, for example a protease, polymerase, exonuclease, helicase, glycosyltransferase, esterase, integrase, reverse transcriptase, kinase, primase, proteinase, methyltransferase, or nucleotidase.

Specific examples of neosubstrates that can be targeted for degradation by the tricyclic compounds of the present invention with rationales for degradation include, but are not limited to the following:
CK1α is a casein kinase that uses acidic proteins such as caseins as phosphorylation substrates. CK1A participates in Wnt signaling and its overexpression is correlated with poor survival in cancer.
GSPT1 (G1 to S phase transition 1) is a translation termination factor. GSPT1 interacts with BIRC2 and is proteolytically processed into an IAP-binding protein. GSPT1 is expressed in cancer tissues including in gastric cancers.
STAT proteins are cytoplasmic transcription factors that can be activated by various extracellular signaling proteins. Stat proteins have been shown to upregulate various genes involved in uncontrolled cellular proliferation, anti-apoptotic responses and/or angiogenesis.
SALL4 (Spalt Like Transcription Factor 4) is a developmental transcription factor which is associated with developmental syndromes and abnormalities such as Duane-Radial Ray Syndrome and Ivic Syndrome.
PLZF (promyelocytic leukemia zinc finger), also known as ZBTB16 (Zinc Finger and BTB Domain Containing 16) is a transcription factor that regulates cellular proliferation, differentiation, organ development, cell maintenance, and immune cell development. PLZF acts as a tumor suppressor in some cancers, however, PLZF is actually an oncoprotein in certain cancers, such as renal cell carcinoma, glioblastoma, and testicular cancer.
p63 (tumor protein p63) is a pleoiotropic protein involved in cellular proliferation, apoptosis, differentiation, and even aging. There are several isoforms of p63. Some forms of p63 will suppressor tumors while other forms and mutants will promote cancer metastasis.
NRAS is a small GTP binding protein in which oncogenic activating mutations drive tumorigenesis. NRAS mutations are found for example in melanoma and thyroid cancer, and sometimes occur at Q61K and Q61R
BRD9 (bromodomain-containing protein 9) is a constituent part of the SWI/SNF (BAF) chromatin remodeling complexes. Mutations in BRD9 have been linked to several cancers and even native BRD9 when overexpressed can be oncogenic. Cancers related to BRD9 include cervical cancer, non-small cell lung cancer, and liver cancer.
P13KCA is a kinase that is one of the most commonly mutated oncogenes across a variety of human cancers including but not limited to breast, endometrial, squamous head and neck and squamous lung. Mutations for example are H1047R, E545K, E542K and sometimes lead to aberrant activation of PI3K-AKT-mTOR pathway.
RET (RET proto-oncogene) is a protein that spans the cellular membrane and interacts with with the cell's environment. RET binds growth factors and triggers complex cascades of chemical reactions within the cell. Non-limiting examples of RET mediated disorders include nonsyndromic paraganglioma, Hirschsprung disease, multiple endocrine neoplasia, lung cancer, and other cancers.
RIT1 is a small GTP binding protein, which is an activating mutation in cancers for example Noonan syndrome (a RAS-opathy), lung cancer and heme malignancies.
MCL1 is a member of the BCL2 family and regulator of apoptosis. Diseases that are associated with MCL1 include myeloid leukemia and chlamydia.
ARID1B is an AT-rich interactive domain-containing protein1. It is a component of SW1/SNF complex and binds DNA non-specifically. It is a top dependency specific to ARID1A mutated cancer cell lines. ARID1A-deficient cancers comprise a high percentage of certain tumors including but not limited to ovarian clear cell carcinoma.
P300 (histone acetyltransferase p300 or p300 HAT) is an enzyme that regulates transcription of genes via chromatin remodeling by mediating the wrapping of histone to DNA. As a result, P300 plays a vital role in cell growth and division. Mutations of P300 can cause various types of cancers including colon, stomach, breast and pancreatic cancer.
ARID2 which is a component of the polybromo-associated BAF (PBAF) chromatin remodeling complex
FAM38 (also known as PIEZO1) is a pore-forming subunit of a non-specific cation channel. As a cation channel subunit FAM38 is involved in the recruitment of R-Ras to the endoplasmic reticulum. Loss of FAM38 has been shown to cause metathesis in small cell lung cancer lines.
NSD2, which belongs to a histone methyltransferase (“HMT”) gene class, is overexpressed by oncogenic fusion transcripts of for example, multiple myeloma, ALL, CLL and MCL.
CSK is a non-receptor tyrosine-protein kinase involved in the regulation of cell growth, differentiation, migration, and immune response.
CBLB is a E3 ubiquitin-protein ligase that accepts ubiquitin from E2 ubiquitin-conjugating enzymes and then transfers it to substrates for degradation.
EGFR (Epidermal Growth Factor Receptor) is a tyrosine kinase receptor. EGFR is associated with the progression of several epithelial malignancies including colorectal cancer, adenocarcinomas (including that of the lung), glioblastoma, and epithelial tumors of the head and neck. Additionally, EGFR can be used as a receptor for entry of a microbial infection or virus such as HCV.
WRN is a RecQ DNA helicase. WRN loss leads to DNA damage in MSI (microsatellite instability) cells, but not MSS (microsatellite stable) cells. This can lead to DSB (double strand break) responses to promote cell death and cell cycle arrest preferentially in MSI cells.
NTRK and its gene fusions (including NTRK1, NTRK2, and NTRK3 gene fusions) are oncogenes for several adult and pediatric cancers. NTRK fusions are a major source of rare cancers such as secretory breast carcinoma, mammary analogue secretory carcinoma, and infantile fibrosarcoma. NTRK fusions can also cause more common cancers as well.
ADAR is RNA specific adenosine deaminase. IFN-stimulated (ISG) signature-positive cancer cells are sensitive to the loss of ADAR, a dsRNA-editing enzyme that is also an ISG. Tumor-derived IFN resulting in chronic signaling creates a cellular state primed to respond to dsRNA accumulation, rendering ISG-positive tumors susceptible to ADAR loss. Loss of ADAR1 overcomes resistance to PD-1 checkpoint blockade caused by inactivation of antigen presentation.
SOS1 promotes generation of active form of KRAS so blocking can counteract upstream or mutant activation of KRAS.
KRAS is a gene that encodes K-Ras which is a protein in the RAS/MAPK pathway that relays extracellular signals to the cell's nucleus. These signals either result in proliferation or differentiation. K-Ras sends signals when it is bound to GTP which acts like a molecular on off switch. KRAS mutations are frequently observed in cecal cancers. K-Ras is implicated in several cancers including colorectal cancer and lung cancer.
WDR5 is a member of the WD repeat protein family. WD repeats are minimally conserved regions of 40 amino acids bracketed by gly-his and trp-asp. WDR5 interacts with host cell factor C₁, MLL, and is a key determinant for MYC recruitment. WD5 is implicated in mixed lineage leukemias.
ALK, including ALK-fusions such as EML-ALK and ALK fusion proteins in which the kinase domain of ALK has been fused to the amino-terminal portion of various proteins have been described in numerous cancers including but not limited to ALCL, IMT, DLBCL, NSCLC, RMC, RCC, breast cancer, colon carcinoma, serous ovarian carcinoma (SOC) and esophageal squamous cell carcinoma (ESCC).
PTPN2 regulates CD₈+ T cell subpopulations and affects tumor immunity.
CTNNB1 (β-Catenin) is involved in cell signaling as part of the Wnt signaling pathway. Proteins in this pathway attach to CTNNB1 and trigger protein migration to the nucleus. CTNNB1 is associated with desmoid tumors, pilomatricoma, Wilm's tumor, aldosterone-producing adenoma, ovarian cancer, and other cancers.
FGFR including FGFR1, FGFR3, or FGFR4 (and fusions), is a receptor tyrosine kinase amplified in numerous cancers including squamous NSCLC, breast, ovarian, bladder, gastric and endometrial
ROS1 is a proto-oncogene receptor tyrosine kinase highly expressed in a variety of tumor cells
MYD88 (myeloid differentiation primary response 88) provides instructions for making a protein involved with signaling in immune cells and its mutation is found in cancer cells
HER2 (human epidermal growth factor receptor 2) is a growth-promoting protein on the outside of breast cells. Even HER2-negative breast cancer cells have HER2, however those with above the normal levels of HER2 are called HER2-positive. HER2 is a very important gene in the treatment of breast cancer. Approximately 1 of every 5 breast cancers has extra copies of the HER2 gene which leads to growth of the cancer cells.
TBXT, which is a transcription factor overexpressed in multiple cancers and correlated with tumor grade and aggressiveness.
PTP4A3 (PRL3) is a protein tyrosine phosphatase IVA3 is a prenylated phosphatase that is involved with cell signaling and overexpression causes cell growth.
MET (including exon-14 skipping mutations) is a receptor tyrosine kinase; alternatively spliced MET receptor exhibits decreased ubiquitination and delayed downregulation, leading to prolonged activation of MET and MAP kinase, which can be transforming.
USP7 is a deubiquitinating enzyme involved in prostate cancer, lung cancer, brain cancer, colon cancer, breast cancer, and other cancers.
NRF2(NFE2L2) is a basic leucine zipper protein that regulates the expression of antioxidant proteins that are cytoprotective; mutation or activation can promote cancer.
SF3B1 is a gene involved in splicing of RNA units. SF3B1 is involved in RNA splicing, mRNA splicing minor pathway, and PKN1 activated stimulation of the Androgen Receptor. Mutations to SF3B1 have been related to various cancers.
Any of the Ikaros family of proteins (IKZF 1, 2, 3, 4, or 5). IKZF 2 (Helios) and IKZF 4 (Eos) are selectively expressed in Treg cells but not effector or memory cells. FoxP₃/IKZF4/CtBP1 forms an inhibitory complex that suppresses gene expression (IL-2, IFN-γ) in Tregs and maintains its suppressive signature. Knocking down IKZF4 in Tregs abrogates the cell's ability to suppress immune responses and enables partial effector function. IKZF2 regulates Treg differentiation through a distinct mechanism from IKZF4. IKZF2 knockout in FoxP₃-expressing Tregs promotes loss of inhibitory properties (with an increase in IL-2) and expression of T-effector cytokines via STAT5 (which regulates FoxP₃). Ikaros family proteins are upregulated in myelogenous leukemia.
MEN1 is a putative tumor suppressor associated with multiple endocrine neoplasia type 1 (MEN-1 syndrome). MEN1 is an autosomal dominant disorder in which affected individuals variably develop tumors in the parathyroids, anterior pituitary, and enteropancreatic endocrine tissue.
JCV proteins are encoded by the JC virus genome. In people with weakened immune systems, the JC virus can cause a serious brain infection called progressive multifocal leukoencephalopathy (PML). PML damages the outer coating of your nerve cells. It can cause permanent disabilities and can even be deadly. The JC virus genome encodes large and small tumor-antigens, the agnoprotein, and capsid proteins VP1 to VP3. The capsid proteins play a role in cellular entry and the agnoprotein plays a role in virion maturation.
CYP17A1 and CYP20A1 are heme proteins and are members of the cytochromes P450 family. The cytochrome P450 proteins are monooxygenases that catalyze many reactions involved in drug metabolism and synthesis of cholesterol, steroids, and other lipids. Many P450s are important enzymes for drug metabolism and other P450s play physiological roles by metabolizing endogenous substrates. For example, CYP17A1 is largely associated with endocrine effects and steroid hormone metabolism and mutations are associated with rare forms of congenital adrenal hyperplasia, specifically 17α-hydroxylase deficiency/17,20-lyase deficiency and isolated 17,20-lyase deficiency. CYP20A1 is an orphan isoform in humans that is expressed in the brain and liver.
BKV proteins are encoded by the BK virus genome. The human polyomavirus BK (BKV) infects humans worldwide and establishes a persistent infection in the kidney. The BK virus genome encodes three regulatory proteins, large and small tumor-antigen and the agnoprotein, as well as the capsid proteins VP1 to VP3. The agnoprotein helps regulate the virus replication and disrupt host cell processes once the viruses enter cells.
MEK½ are extracellular signal-regulated kinases that participate in the Ras/Raf/MEK/ERK pathway, a signaling cascade that regulates various cellular processes such as proliferation, differentiation, and cell cycle progression in response to a variety of extracellular signals. Overactivation or mutations in this pathway is linked to many cancers and the inhibition of MEK blocks cell proliferation leading to apoptosis. For example, β3-αC loop MEK1 mutants exhibit a strong oncogenic potential, but differential sensitivity to MEK inhibitors in clinic therapy or trials.
Ataxin-2 is a member of the Like-Sm (LSm) protein family and participates in a large number of functions related to RNA processing and RNA metabolism. Mutations in ATXN2 cause the neurodegenerative disease spinocerebellar ataxia type 2 (SCA2).
JAK2 is a non-receptor tyrosine kinase and a member of the Janus kinase family. It has been implicated in signaling by members of the type II cytokine receptor family, the GM-CSF receptor family, the gp130 receptor family, and the single chain receptors. JAK2 gene fusions with the TEL(ETV6) (TEL-JAK2) and PCM1 genes have been found in patients suffering leukemia and mutations in JAK2 have been implicated in polycythemia vera, essential thrombocythemia, and myelofibrosis as well as other myeloproliferative disorders.
PTPN11(SHP2) is a non-receptor tyrosine phosphatase that serves as a mediator of RTK-signaling. Overexpression has been observed in cancers with recurrent mutations observed in AML, JMML, and neuroblastoma; loss or inhibition of SHP2 has been shown to suppress proliferation of AML or other RTK-driven cancers
ERK1/ERK2 are extracellular signal-regulated kinases that participate in the Ras/Raf/MEK/ERK pathway, a signaling cascade that regulates various cellular processes such as proliferation, differentiation, and cell cycle progression in response to a variety of extracellular signals. Overactivation of this pathway is linked to many cancers.
BRAF Type II mutants are BRAF mutations classified as “constitutive active RAS-independent dimers with high or intermediate BRAF kinase activity involving codons outside 600, including BRAF fusion mutants.” Patients with Type II mutants typically have shorter survival times than those with Type I mutants and the cancer can be more aggressive.
ERBB3 is a transmembrane pseudo-RTK with strong genetic links to cancer
GRB2 is a scaffold adapter involved in RTK signaling to downstream pathways including MAPK. GRB2 recruits to a variety of signaling molecules to receptors to form multimeric signaling complexes that lead to cellular responses such as proliferation and invasion, and is therefore linked to cancer and tumorigenesis.
CBP is a transcriptional coactivator involved in the transcriptional coactivation of many different transcription factors and is therefore involved in a wide array of cellular activities, such as DNA repair, cell growth, differentiation and apoptosis. CPB is also referred to as CREBBP. It is a CREB-binding protein transcriptional coactivator, and is associated with a range of cancers including leukemia, NSCLC, HCV-associated hepatocellular carcinoma, melanoma, lung, lymphoma and bladder.
ATAD2 and ATAD2B are bromo/ATP helicase which can be a transcriptional co-activator of the nuclear receptor ESR1 required to induce a subset of estradiol target genes and can play a role in triple negative breast cancer
BAP1 is a deubiquitinating enzyme that can function as a tumor suppressor and a metastasis suppressor in cancer. BAP1's ability to regulate gene environment interactions in carcinogenesis has been linked to its dual role in the nucleus and in the cytoplasm. In the nucleus, BAP1 modulates the transcriptional regulation of several gene programs and promotes DNA repair by facilitating homologous recombination. It is found in PBRM1-deficient CRC.
BRPF1 is a bromodomain containing histone reader that associates with Moz and Morf, bearing HAT activity for H3. BRPF1 plays a role in cancer such as hematopoietic cancer including leukemia.
BRD4 is an epigenetic reader and a member of the BET protein family. BRD4 binds acetylated histones and plays a central role in controlling cellular gene transcription and proliferation and is therefore important in angiogenesis and the development of inflammation-associated diseases, cardiovascular diseases, central nervous system disorders and cancers.
EPAS1(HIF2a) belongs to a group of transcription factors involved in the physiological response to oxygen concentration and is encoded under hypoxic conditions. It is also important in the development of the heart, and for maintaining the catecholamine balance required for protection of the heart. Mutation often leads to neuroendocrine tumors, such as such as paragangliomas, somatostatinomas and/or pheochromocytomas.
KMT2D is a histone methyltransferase with a strong genetic link to cancer. The protein co-localizes with lineage determining transcription factors on transcriptional enhancers and is essential for cell differentiation and embryonic development. It also plays critical roles in regulating cell fate transition, metabolism, and tumor suppression.
Menin is a scaffolding protein that binds in a bidentate fashion to N-terminus of KMT2A (MLL) and MLL-fusion proteins, enabling binding and localization to chromatin; linked to leukemia and other cancers
MLLT1(ENL) is a YEATS domain containing protein; plays a role in transcriptional initiation/elongation (YEATS domain dependent) and a key interactor with DOTiL
DOT1L is a histone H3K79 methyltransferase that methylates lysine 79 on histone H3, an evolutionarily conserved methylation mark. DOT1L is involved in a number of key processes ranging from gene expression to DNA-damage response and cell cycle progression. DOT1L has also been implicated in the development of mixed lineage leukemia (MLL)-rearranged leukemia.
NSD2 is a histone methyltransferase that is expressed ubiquitously in early development and overexpressed in cancer cells, including in ALL, CLL and MCL.
TAU are the six highly soluble protein isoforms produced by alternative splicing from the gene MAPT (microtubule-associated protein tau). TAU proteins have roles primarily in maintaining the stability of microtubules in axons and are abundant in the neurons of the central nervous system (CNS). Pathologies and dementias of the nervous system such as Alzheimer's disease and Parkinson's disease are associated with tau proteins that have become hyperphosphorylated insoluble aggregates called neurofibrillary tangles.
HTT is the Huntington protein. HTT is essential for development and is highly expressed in neurons and testes. Huntingtin upregulates the expression of Brain Derived Neurotrophic Factor (BDNF) at the transcription level, and its mutated form leads to Huntington Disease.
NSD3 is a histone methyltransferase and a driver of 8p11-12 amplification found in cancers including squamous lung cancer, breast cancer and AML
SNCA is a member of the synuclein family, and is involved in regulation of dopamine release and transport, fibrillization of microtubule associated protein tau, and neuroprotective phenotype in non-dopaminergic neurons. Mutation of SNCA is related to neurodegenerative diseases, such as Parkinson's disease, Alzheimer's disease (AD), Lewy bodies' disease (LBD) and Muscular System Atrophy (MSA).
SMARCA2 and SMARCA4 are proteins encoded by the SWI/SNF family of proteins that have helicase and ATPase activities and regulate transcription of genes by altering chromatin structure as an ATP-dependent chromatin remodeler
BTK is a tyrosine kinase that plays a crucial role in the oncogenic signaling that is critical for proliferation and survival of leukemic cells in many B cell malignancies. BTK was initially shown to be mutated in the primary immunodeficiency X-linked agammaglobulinemia (XLA) and is essential at various stages of B lymphocyte development.
TAF1 is a TBP-associated factor with kinase domains, acetyltransferase and bromodomains. TAF1 is an important component of the transcription factor II D complex that serves a vital function during transcription initiation. Variants of the TAF1 gene have been associated with neurodevelopmental disorders, including intellectual disabilities.
IRAK4 is a threonine/serine protein kinase involved in signaling innate immune responses from Toll-like receptors (TLR). The loss of IRAK4 or its intrinsic kinase activity can entirely stop signaling through the TLR pathways, and is therefore relevant for various inflammatory disorders including rheumatoid arthritis, inflammatory bowel disease and other autoimmune diseases.
SARM1 is a negative regulator of Toll-Like Receptor-activated transcriptional programs.

After axon injury, SARM1 initiates a “self-destruct” mechanism to degrade the metabolite NAD+. This results in metabolic failure in neurons, leading to axon degeneration.
PPM1D (WIP1) is an oncoprotein and a member of the PP2C family of Ser/Thr protein phosphatases. PPM1D is a negative regulator of the cell stress response pathway and is amplified in various cancers, including breast, esophageal, colon, hematological, thyroid, sarcomas, lung, an ovarian.

### Non-Limiting Examples of Diseases that can be Treated with the Tricyclic Compounds

Any of the compounds described herein can be used in an effective amount to treat a host, including a human, in need thereof, optionally in a pharmaceutically acceptable carrier to treat any of the disorders described herein. In certain embodiments, the method comprises administering an effective amount of the active compound or its salt as described herein, optionally including a pharmaceutically acceptable excipient, carrier, or adjuvant (i.e., a pharmaceutically acceptable composition), optionally in combination or alternation with an additional therapeutically active agent or combination of agents.

In certain embodiments a compound of Formula I is used to treat a disorder described herein.

In certain embodiments a compound of Formula II is used to treat a disorder described herein.

In certain embodiments a compound of Formula III is used to treat a disorder described herein.

In certain embodiments a compound of Formula IV is used to treat a disorder described herein.

In certain embodiments a compound of Formula V is used to treat a disorder described herein.

In certain embodiments a compound of Formula VI is used to treat a disorder described herein.

In certain embodiments a compound of Formula VII is used to treat a disorder described herein.

In certain embodiments a compound of Formula VIII is used to treat a disorder described herein.

In certain embodiments a compound of Formula IX is used to treat a disorder described herein.

In certain embodiments a compound of Formula X is used to treat a disorder described herein.

In certain embodiments a compound of Formula XI is used to treat a disorder described herein.

In certain embodiments a compound of Formula XII is used to treat a disorder described herein.

In certain embodiments a compound of Formula XIII is used to treat a disorder described herein.

In certain embodiments a compound of Formula XIV is used to treat a disorder described herein.

In certain embodiments a compound of Formula XV is used to treat a disorder described herein.

In certain embodiments a compound of Formula XVI is used to treat a disorder described herein.

In certain embodiments a compound of Formula XVII is used to treat a disorder described herein.

In certain embodiments the disorder treated by a compound of the present invention is an immunomodulatory disorder. In certain embodiments the disorder treated by a compound of the present invention is mediated by angiogenesis. In certain embodiments the disorder treated by a compound of the present invention is related to the lymphatic system.

In certain embodiments, the method comprises administering an effective amount of the compound as described herein, optionally including a pharmaceutically acceptable excipient, carrier, adjuvant (i.e., a pharmaceutically acceptable composition), optionally in combination or alternation with an additional therapeutically active agent or combination of agents.

In certain embodiments, a compound of the present invention is used to treat a disorder including, but not limited to, benign growth, neoplasm, tumor, cancer, abnormal cellular proliferation, immune disorder, inflammatory disorder, graft-versus-host rejection, viral infection, bacterial infection, an amyloid-based proteinopathy, a proteinopathy, or a fibrotic disorder.

The term “disease state” or “condition” when used in connection with any of the compounds is meant to refer to any disease state or condition that is responsive to a compound of the present invention, such as cellular proliferation, and where the administration of a compound of the present invention in a patient may provide beneficial therapy or relief of symptoms to a patient in need thereof. In certain instances, the disease state or condition may be cured.

In certain embodiments, a compound or its corresponding pharmaceutically acceptable salt, isotopic derivative, or prodrug as described herein can be used in an effective amount to treat a host, for example a human, with a lymphoma or lymphocytic or myelocytic proliferation disorder or abnormality. For example, a compound as described herein can be administered to a host suffering from a Hodgkin Lymphoma or a Non-Hodgkin Lymphoma. For example, the host can be suffering from a Non-Hodgkin Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); diffuse small-cleaved cell lymphoma (DSCCL); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; Langerhans cell histiocytosis; or Waldenstrom's Macroglobulinemia.

In another embodiment, a compound or its corresponding pharmaceutically acceptable salt, isotopic derivative, or prodrug as described herein can be used in an effective amount to treat a host, for example a human, with a Hodgkin lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin Lymphoma; or Nodular Lymphocyte Predominant HL.

In another embodiment, a compound or its corresponding pharmaceutically acceptable salt, isotopic derivative, or prodrug as described herein can be used in an effective amount to treat a host, for example a human, with an immunomodulatory condition. Non-limiting examples of immunomodulatory conditions include: arthritis, lupus, celiac disease, Sjogren's syndrome, polymyalgia rheumatica, multiple sclerosis, ankylosing spondylitis, type 1 diabetes, alopecia areata, vasculitis, and temporal arteritis.

In certain embodiments, the condition treated with a compound of the present invention is a disorder related to abnormal cellular proliferation. Abnormal cellular proliferation, notably hyperproliferation, can occur as a result of a wide variety of factors, including genetic mutation, infection, exposure to toxins, autoimmune disorders, and benign or malignant tumor induction.

Abnormal proliferation of B-cells, T-cells, and/or NK cells can result in a wide range of diseases such as cancer, proliferative disorders and inflammatory/immune diseases. A host, for example a human, afflicted with any of these disorders can be treated with an effective amount of a compound as described herein to achieve a decrease in symptoms (palliative agent) or a decrease in the underlying disease (a disease modifying agent).

In certain embodiments, a compound or its corresponding pharmaceutically acceptable salt, isotopic derivative, or prodrug as described herein can be used in an effective amount to treat a host, for example a human, with a specific B-cell lymphoma or proliferative disorder such as, but not limited to: multiple myeloma; Diffuse large B cell lymphoma; Follicular lymphoma; Mucosa-Associated Lymphatic Tissue lymphoma (MALT); Small cell lymphocytic lymphoma; diffuse poorly differentiated lymphocytic lymphoma; Mediastinal large B cell lymphoma; Nodal marginal zone B cell lymphoma (NMZL); Splenic marginal zone lymphoma (SMZL); Intravascular large B-cell lymphoma; Primary effusion lymphoma; or Lymphomatoid granulomatosis; B-cell prolymphocytic leukemia; Hairy cell leukemia; Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; T cell/histiocyte rich large B-cell lymphoma; DLBCL associated with chronic inflammation; Epstein-Barr virus (EBV)+ DLBCL of the elderly; Primary mediastinal (thymic) large B-cell lymphoma; Primary cutaneous DLBCL, leg type; ALK+ large B-cell lymphoma; Plasmablastic lymphoma; Large B-cell lymphoma arising in HHV8-associated multicentric; Castleman disease; B-cell lymphoma, unclassifiable, with features intermediate between diffuse large B-cell lymphoma; or B-cell lymphoma, unclassifiable, with features intermediate between diffuse large B-cell lymphoma and classical Hodgkin lymphoma.

In certain embodiments, a compound or its corresponding pharmaceutically salt, isotopic derivative, or prodrug as described herein can be used in an effective amount to treat a host, for example a human, with a T-cell or NK-cell lymphoma such as, but not limited to: anaplastic lymphoma kinase (ALK) positive, ALK negative anaplastic large cell lymphoma, or primary cutaneous anaplastic large cell lymphoma; angioimmunoblastic lymphoma; cutaneous T-cell lymphoma, for example mycosis fungoides, Sézary syndrome, primary cutaneous anaplastic large cell lymphoma, primary cutaneous CD30+ T-cell lymphoproliferative disorder; primary cutaneous aggressive epidermotropic CD8+ cytotoxic T-cell lymphoma; primary cutaneous gamma-delta T-cell lymphoma; primary cutaneous small/medium CD4+ T-cell lymphoma, and lymphomatoid papulosis; Adult T-cell Leukemia/Lymphoma (ATLL); Blastic NK-cell Lymphoma; Enteropathy-type T-cell lymphoma; Hematosplenic gamma-delta T-cell Lymphoma; Lymphoblastic Lymphoma; Nasal NK/T-cell Lymphomas; Treatment-related T-cell lymphomas; for example lymphomas that appear after solid organ or bone marrow transplantation; T-cell prolymphocytic leukemia; T-cell large granular lymphocytic leukemia; Chronic lymphoproliferative disorder of NK-cells; Aggressive NK cell leukemia; Systemic EBV+ T-cell lymphoproliferative disease of childhood (associated with chronic active EBV infection); Hydroa vacciniforme-like lymphoma; Adult T-cell leukemia/lymphoma; Enteropathy-associated T-cell lymphoma; Hepatosplenic T-cell lymphoma; or Subcutaneous panniculitis-like T-cell lymphoma.

In certain embodiments, a compound or its corresponding pharmaceutically acceptable salt, isotopic derivative, or prodrug as described herein can be used to treat a host, for example a human, with leukemia. For example, the host may be suffering from an acute or chronic leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocytic leukemia (a subtype of AML); large granular lymphocytic leukemia; or Adult T-cell chronic leukemia. In certain embodiments, the patient suffers from an acute myelogenous leukemia, for example an undifferentiated AML (M0); myeloblastic leukemia (M1; with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryoblastic leukemia (M7).

There are a number of skin disorders associated with cellular hyperproliferation. Psoriasis, for example, is a benign disease of human skin generally characterized by plaques covered by thickened scales. The disease is caused by increased proliferation of epidermal cells of unknown cause. Chronic eczema is also associated with significant hyperproliferation of the epidermis. Other diseases caused by hyperproliferation of skin cells include atopic dermatitis, lichen planus, warts, pemphigus vulgaris, actinic keratosis, basal cell carcinoma and squamous cell carcinoma.

Other hyperproliferative cell disorders include blood vessel proliferation disorders, fibrotic disorders, autoimmune disorders, graft-versus-host rejection, tumors and cancers.

Blood vessel proliferative disorders include angiogenic and vasculogenic disorders. Proliferation of smooth muscle cells in the course of development of plaques in vascular tissue cause, for example, restenosis, retinopathies and atherosclerosis. Both cell migration and cell proliferation play a role in the formation of atherosclerotic lesions.

Fibrotic disorders are often due to the abnormal formation of an extracellular matrix. Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis.

Mesangial disorders are brought about by abnormal proliferation of mesangial cells. Mesangial hyperproliferative cell disorders include various human renal diseases, such as glomerulonephritis, diabetic nephropathy, malignant nephrosclerosis, thrombotic micro-angiopathy syndromes, transplant rejection, and glomerulopathies.

Another disease with a proliferative component is rheumatoid arthritis. Rheumatoid arthritis is generally considered an autoimmune disease that is thought to be associated with activity of autoreactive T cells, and to be caused by autoantibodies produced against collagen and IgE.

Other disorders that can include an abnormal cellular proliferative component include Bechet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

A compound or its pharmaceutically acceptable salt, isotopic analog, or prodrug as described herein can be used in an effective amount to treat a host, for example a human, with a proliferative condition such as myeloproliferative disorder (MPD), polycythemia vera (PV), essential thrombocythemia (ET), myeloid metaplasia with myelofibrosis (MMM), chronic myelomonocytic leukemia (CMML), hypereosinophilic syndrome (HES), system mast cell disease (SMCD), and the like. In another embodiment, a compound provided herein is useful for the treatment of primary myelofibrosis, post-polycythemia vera myelofibrosis, post-essential thrombocythemia myelofibrosis, and secondary acute myelogenous leukemia.

In certain embodiments, a compound or its pharmaceutically acceptable salt, isotopic analog, or prodrug as described herein can be used in an effective amount to treat a host, for example a human, with a myelodysplastic syndrome (MDS) such as, but not limited to: refractory cytopenia with unilineage dysplasia, refractory anemia with ring sideroblasts (RARS), refractory anemia with ring sideroblasts—thrombocytosis (RARS-t), refractory cytopenia with multilineage dyslplasia (RCMD) including RCMD with multilineage dysplasia and ring sideroblasts (RCMD-RS), Refractory amenias with excess blasts I (RAEB-I) and II (RAEB-II), 5q-syndrome, refractory cytopenia of childhood, and the like.

The term “neoplasia” or “cancer” is used to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Exemplary cancers which may be treated by the present compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present invention include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

Additional cancers which may be treated using the disclosed compounds according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+ DLBCL of the elderly; primary mediastinal (thymic) large B-cell lymphoma, primary cutaneous DLBCL, leg type, ALK+ large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma. In certain embodiments the disorder is adenoid cystic carcinoma. In certain embodiments the disorder is NUT midline carcinoma.

In another embodiment, a compound or its pharmaceutically acceptable salt, isotopic derivative or prodrug as described herein can be used in an effective amount to treat a host, for example a human, with an autoimmune disorder. Examples include, but are not limited to: Acute disseminated encephalomyelitis (ADEM); Addison's disease; Agammaglobulinemia; Alopecia areata; Amyotrophic lateral sclerosis (Also Lou Gehrig's disease; Motor Neuron Disease); Ankylosing Spondylitis; Antiphospholipid syndrome; Antisynthetase syndrome; Atopic allergy; Atopic dermatitis; Autoimmune aplastic anemia; Autoimmune arthritis; Autoimmune cardiomyopathy; Autoimmune enteropathy; Autoimmune granulocytopenia; Autoimmune hemolytic anemia; Autoimmune hepatitis; Autoimmune hypoparathyroidism; Autoimmune inner ear disease; Autoimmune lymphoproliferative syndrome; Autoimmune myocarditis; Autoimmune pancreatitis; Autoimmune peripheral neuropathy; Autoimmune ovarian failure; Autoimmune polyendocrine syndrome; Autoimmune progesterone dermatitis; Autoimmune thrombocytopenic purpura; Autoimmune thyroid disorders; Autoimmune urticarial; Autoimmune uveitis; Autoimmune vasculitis; Balo disease/Balo concentric sclerosis; Behget's disease; Berger's disease; Bickerstaffs encephalitis; Blau syndrome; Bullous pemphigoid; Cancer; Castleman's disease; Celiac disease; Chagas disease; Chronic inflammatory demyelinating polyneuropathy; Chronic inflammatory demyelinating polyneuropathy; Chronic obstructive pulmonary disease; Chronic recurrent multifocal osteomyelitis; Churg-Strauss syndrome; Cicatricial pemphigoid; Cogan syndrome; Cold agglutinin disease; Complement component 2 deficiency; Contact dermatitis; Cranial arteritis; CREST syndrome; Crohn's disease; Cushing's Syndrome; Cutaneous leukocytoclastic angiitis; Dego's disease; Dercum's disease; Dermatitis herpetiformis; Dermatomyositis; Diabetes mellitus type 1; Diffuse cutaneous systemic sclerosis; Discoid lupus erythematosus; Dressler's syndrome; Drug-induced lupus; Eczema; Endometriosis; Enthesitis-related arthritis; Eosinophilic fasciitis; Eosinophilic gastroenteritis; Eosinophilic pneumonia; Epidermolysis bullosa acquisita; Erythema nodosum; Erythroblastosis fetalis; Essential mixed cryoglobulinemia; Evan's syndrome; Extrinsic and intrinsic reactive airways disease (asthma); Fibrodysplasia ossificans progressive; Fibrosing alveolitis (or Idiopathic pulmonary fibrosis); Gastritis; Gastrointestinal pemphigoid; Glomerulonephritis; Goodpasture's syndrome; Graves' disease; Guillain-Barre syndrome (GBS); Hashimoto's encephalopathy; Hashimoto's thyroiditis; Hemolytic anemia; Henoch-Schonlein purpura; Herpes gestationis (Gestational Pemphigoid); Hidradenitis suppurativa; Hughes-Stovin syndrome; Hypogammaglobulinemia; Idiopathic inflammatory demyelinating diseases; Idiopathic pulmonary fibrosis; Idiopathic thrombocytopenic purpura; IgA nephropathy; Immune glomerulonephritis; Immune nephritis; Immune pneumonitis; Inclusion body myositis; inflammatory bowel disease; Interstitial cystitis; Juvenile idiopathic arthritis aka Juvenile rheumatoid arthritis; Kawasaki's disease; Lambert-Eaton myasthenic syndrome; Leukocytoclastic vasculitis; Lichen planus; Lichen sclerosus; Linear IgA disease (LAD); Lupoid hepatitis aka Autoimmune hepatitis; Lupus erythematosus; Majeed syndrome; microscopic polyangiitis; Miller-Fisher syndrome; mixed connective tissue disease; Morphea; Mucha-Habermann disease aka Pityriasis lichenoides et varioliformis acuta; Multiple sclerosis; Myasthenia gravis; Myositis; Ménière's disease; Narcolepsy; Neuromyelitis optica (also Devic's disease); Neuromyotonia; Occular cicatricial pemphigoid; Opsoclonus myoclonus syndrome; Ord's thyroiditis; Palindromic rheumatism; PANDAS (pediatric autoimmune neuropsychiatric disorders associated with streptococcus); Paraneoplastic cerebellar degeneration; Paroxysmal nocturnal hemoglobinuria (PNH); Parry Romberg syndrome; Pars planitis; Parsonage-Turner syndrome; Pemphigus vulgaris; Perivenous encephalomyelitis; Pernicious anaemia; POEMS syndrome; Polyarteritis nodosa; Polymyalgia rheumatic; Polymyositis; Primary biliary cirrhosis; Primary sclerosing cholangitis; Progressive inflammatory neuropathy; Psoriasis; Psoriatic arthritis; pure red cell aplasia; Pyoderma gangrenosum; Rasmussen's encephalitis; Raynaud phenomenon; Reiter's syndrome; relapsing polychondritis; restless leg syndrome; retroperitoneal fibrosis; rheumatic fever; rheumatoid arthritis; Sarcoidosis; Schizophrenia; Schmidt syndrome; Schnitzler syndrome; Scleritis; Scleroderma; Sclerosing cholangitis; serum sickness; Sjögren's syndrome; Spondyloarthropathy; Stiff person syndrome; Still's disease; Subacute bacterial endocarditis (SBE); Susac's syndrome; Sweet's syndrome; Sydenham chorea; sympathetic ophthalmia; systemic lupus erythematosus; Takayasu's arteritis; temporal arteritis (also known as “giant cell arteritis”); thrombocytopenia; Tolosa-Hunt syndrome; transverse myelitis; ulcerative colitis; undifferentiated connective tissue disease; undifferentiated spondyloarthropathy; urticarial vasculitis; vasculitis and vitiligo.

In another embodiment, a viral disease is treated, for example, SARS-CoV1, SARS-CoV2, Coronaviridae, Flaviviridae, Dengue, West Nile, RSV, Epstein Barr Virus (EBV), Hepatitis B, Hepatitis C, HIV, HTLV 1, Varicella-Zoster Virus (VZV) and Human Papilloma Virus (HPV); or Wegener's granulomatosis. In some embodiments, the autoimmune disease is an allergic condition, including those from asthma, food allergies, atopic dermatitis, chronic pain, and rhinitis.

Cutaneous contact hypersensitivity and asthma are just two examples of immune responses that can be associated with significant morbidity. Others include atopic dermatitis, eczema, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, cutaneous lupus erythematosus, scleroderma, vaginitis, proctitis, and drug eruptions. These conditions may result in any one or more of the following symptoms or signs: itching, swelling, redness, blisters, crusting, ulceration, pain, scaling, cracking, hair loss, scarring, or oozing of fluid involving the skin, eye, or mucosal membranes.

In atopic dermatitis, and eczema in general, immunologically mediated leukocyte infiltration (particularly infiltration of mononuclear cells, lymphocytes, neutrophils, and eosinophils) into the skin importantly contributes to the pathogenesis of these diseases. Chronic eczema also is associated with significant hyperproliferation of the epidermis. Immunologically mediated leukocyte infiltration also occurs at sites other than the skin, such as in the airways in asthma and in the tear producing gland of the eye in keratoconjunctivitis sicca.

A compound or its pharmaceutically acceptable salt, isotopic variant, or prodrug as described herein can be used in an effective amount to treat a host, for example a human, with a skin disorder such as psoriasis (for example, psoriasis vulgaris), atopic dermatitis, skin rash, skin irritation, skin sensitization (e.g., contact dermatitis or allergic contact dermatitis). For example, certain substances including some pharmaceuticals when topically applied can cause skin sensitization. In some embodiments, the skin disorder is treated by topical administration of compounds known in the art in combination with the compounds disclosed herein. In one non-limiting embodiment compounds of the present invention are used as topical agents in treating contact dermatitis, atopic dermatitis, eczematous dermatitis, psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, and drug eruptions. The novel method may also be useful in reducing the infiltration of skin by malignant leukocytes in diseases such as mycosis fungoides.

Disease states of conditions which may be treated using compounds according to the present invention include, for example, asthma, autoimmune diseases such as multiple sclerosis, various cancers, ciliopathies, cleft palate, diabetes, heart disease, hypertension, inflammatory bowel disease, mental retardation, mood disorder, obesity, refractive error, infertility, Angelman syndrome, Canavan disease, Coeliac disease, Charcot-Marie-Tooth disease, Cystic fibrosis, Duchenne muscular dystrophy, Haemochromatosis, Haemophilia, Klinefelter's syndrome, Neurofibromatosis, Phenylketonuria, Polycystic kidney disease 1 (PKD1) or 2 (PKD2) Prader-Willi syndrome, Sickle-cell disease, Tay-Sachs disease, Turner syndrome.

Further disease states or conditions which may be treated by compounds according to the present invention include Alzheimer's disease, Amyotrophic lateral sclerosis (Lou Gehrig's disease), Anorexia nervosa, Anxiety disorder, Atherosclerosis, Attention deficit hyperactivity disorder, Autism, Bipolar disorder, Chronic fatigue syndrome, Chronic obstructive pulmonary disease, Crohn's disease, Coronary heart disease, Dementia, Depression, Diabetes mellitus type 1, Diabetes mellitus type 2, Epilepsy, Guillain-Barre syndrome, Irritable bowel syndrome, Lupus, Metabolic syndrome, Multiple sclerosis, Myocardial infarction, Obesity, Obsessive-compulsive disorder, Panic disorder, Parkinson's disease, Psoriasis, Rheumatoid arthritis, Sarcoidosis, Schizophrenia, Stroke, Thromboangiitis obliterans, Tourette syndrome, Vasculitis.

Still additional disease states or conditions which can be treated by compounds according to the present invention include aceruloplasminemia, Achondrogenesis type II, achondroplasia, Acrocephaly, Gaucher disease type 2, acute intermittent porphyria, Canavan disease, Adenomatous Polyposis Coli, ALA dehydratase deficiency, adenylosuccinate lyase deficiency, Adrenogenital syndrome, Adrenoleukodystrophy, ALA-D porphyria, ALA dehydratase deficiency, Alkaptonuria, Alexander disease, Alkaptonuric ochronosis, alpha 1-antitrypsin deficiency, alpha-1 proteinase inhibitor, emphysema, amyotrophic lateral sclerosis Alstrom syndrome, Alexander disease, Amelogenesis imperfecta, ALA dehydratase deficiency, Anderson-Fabry disease, androgen insensitivity syndrome, Anemia Angiokeratoma Corporis Diffusum, Angiomatosis retinae (von Hippel-Lindau disease) Apert syndrome, Arachnodactyly (Marfan syndrome), Stickler syndrome, Arthrochalasis multiplex congenital (Ehlers-Danlos syndrome #arthrochalasia type) ataxia telangiectasia, Rett syndrome, primary pulmonary hypertension, Sandhoff disease, neurofibromatosis type II, Beare-Stevenson cutis gyrata syndrome, Mediterranean fever, familial, Benjamin syndrome, beta-thalassemia, Bilateral Acoustic Neurofibromatosis (neurofibromatosis type II), factor V Leiden thrombophilia, Bloch-Sulzberger syndrome (incontinentia pigmenti), Bloom syndrome, X-linked sideroblastic anemia, Bonnevie-Ullrich syndrome (Turner syndrome), Bourneville disease (tuberous sclerosis), prion disease, Birt-Hogg-Dubé syndrome, Brittle bone disease (osteogenesis imperfecta), Broad Thumb-Hallux syndrome (Rubinstein-Taybi syndrome), Bronze Diabetes/Bronzed Cirrhosis (hemochromatosis), Bulbospinal muscular atrophy (Kennedy's disease), Burger-Grutz syndrome (lipoprotein lipase deficiency), CGD Chronic granulomatous disorder, Campomelic dysplasia, biotinidase deficiency, Cardiomyopathy (Noonan syndrome), Cri du chat, CAVD (congenital absence of the vas deferens), Caylor cardiofacial syndrome (CBAVD), CEP (congenital erythropoietic porphyria), cystic fibrosis, congenital hypothyroidism, Chondrodystrophy syndrome (achondroplasia), otospondylomegaepiphyseal dysplasia, Lesch-Nyhan syndrome, galactosemia, Ehlers-Danlos syndrome, Thanatophoric dysplasia, Coffin-Lowry syndrome, Cockayne syndrome, (familial adenomatous polyposis), Congenital erythropoietic porphyria, Congenital heart disease, Methemoglobinemia/Congenital methaemoglobinaemia, achondroplasia, X-linked sideroblastic anemia, Connective tissue disease, Conotruncal anomaly face syndrome, Cooley's Anemia (beta-thalassemia), Copper storage disease (Wilson's disease), Copper transport disease (Menkes disease), hereditary coproporphyria, Cowden syndrome, Craniofacial dysarthrosis (Crouzon syndrome), Creutzfeldt-Jakob disease (prion disease), Cockayne syndrome, Cowden syndrome, Curschmann-Batten-Steinert syndrome (myotonic dystrophy), Beare-Stevenson cutis gyrata syndrome, primary hyperoxaluria, spondyloepimetaphyseal dysplasia (Strudwick type), muscular dystrophy, Duchenne and Becker types (DBMD), Usher syndrome, Degenerative nerve diseases including de Grouchy syndrome and Dejerine-Sottas syndrome, developmental disabilities, distal spinal muscular atrophy, type V, androgen insensitivity syndrome, Diffuse Globoid Body Sclerosis (Krabbe disease), Di George's syndrome, Dihydrotestosterone receptor deficiency, androgen insensitivity syndrome, Down syndrome, Dwarfism, erythropoietic protoporphyria Erythroid 5-aminolevulinate synthetase deficiency, Erythropoietic porphyria, erythropoietic protoporphyria, erythropoietic uroporphyria, Friedreich's ataxia-familial paroxysmal polyserositis, porphyria cutanea tarda, familial pressure responsiveneuropathy, primary pulmonary hypertension (PPH), Fibrocystic disease of the pancreas, fragile X syndrome, galactosemia, genetic brain disorders, Giant cell hepatitis (Neonatal hemochromatosis), Gronblad-Strandberg syndrome (pseudoxanthoma elasticum), Gunther disease (congenital erythropoietic porphyria), haemochromatosis, Hallgren syndrome, sickle cell anemia, hemophilia, hepatoerythropoietic porphyria (HEP), Hippel-Lindau disease (von Hippel-Lindau disease), Huntington's disease, Hutchinson-Gilford progeria syndrome (progeria), Hyperandrogenism, Hypochondroplasia, Hypochromic anemia, Immune system disorders, including X-linked severe combined immunodeficiency, Insley-Astley syndrome, Jackson-Weiss syndrome, Joubert syndrome, Lesch-Nyhan syndrome, Jackson-Weiss syndrome, Kidney diseases, including hyperoxaluria, Klinefelter's syndrome, Kniest dysplasia, Lacunar dementia, Langer-Saldino achondrogenesis, ataxia telangiectasia, Lynch syndrome, Lysyl-hydroxylase deficiency, Machado-Joseph disease, Metabolic disorders, including Kniest dysplasia, Marfan syndrome, Movement disorders, Mowat-Wilson syndrome, cystic fibrosis, Muenke syndrome, Multiple neurofibromatosis, Nance-Insley syndrome, Nance-Sweeney chondrodysplasia, Niemann-Pick disease, Noack syndrome (Pfeiffer syndrome), Osler-Weber-Rendu disease, Peutz-Jeghers syndrome, Polycystic kidney disease, polyostotic fibrous dysplasia (McCune-Albright syndrome), Peutz-Jeghers syndrome, Prader-Labhart-Willi syndrome, hemochromatosis, primary hyperuricemia syndrome (Lesch-Nyhan syndrome), primary pulmonary hypertension, primary senile degenerative dementia, prion disease, progeria (Hutchinson Gilford Progeria Syndrome), progressive chorea, chronic hereditary (Huntington) (Huntington's disease), progressive muscular atrophy, spinal muscular atrophy, propionic acidemia, protoporphyria, proximal myotonic dystrophy, pulmonary arterial hypertension, PXE (pseudoxanthoma elasticum), Rb (retinoblastoma), Recklinghausen disease (neurofibromatosis type I), Recurrent polyserositis, Retinal disorders, Retinoblastoma, Rett syndrome, RFALS type 3, Ricker syndrome, Riley-Day syndrome, Roussy-Levy syndrome, severe achondroplasia with developmental delay and acanthosis nigricans (SADDAN), Li-Fraumeni syndrome, sarcoma, breast, leukemia, and adrenal gland (SBLA) syndrome, sclerosis tuberose (tuberous sclerosis), SDAT, SED congenital (spondyloepiphyseal dysplasia congenita), SED Strudwick (spondyloepimetaphyseal dysplasia, Strudwick type), SEDc (spondyloepiphyseal dysplasia congenita) SEMD, Strudwick type (spondyloepimetaphyseal dysplasia, Strudwick type), Shprintzen syndrome, Skin pigmentation disorders, Smith-Lemli-Opitz syndrome, South-African genetic porphyria (variegate porphyria), infantile-onset ascending hereditary spastic paralysis, Speech and communication disorders, sphingolipidosis, Tay-Sachs disease, spinocerebellar ataxia, Stickler syndrome, stroke, androgen insensitivity syndrome, tetrahydrobiopterin deficiency, beta-thalassemia, Thyroid disease, Tomaculous neuropathy (hereditary neuropathy with liability to pressure palsies), Treacher Collins syndrome, Triplo X syndrome (triple X syndrome), Trisomy 21 (Down syndrome), Trisomy X, VHL syndrome (von Hippel-Lindau disease), Vision impairment and blindness (Alstrom syndrome), Vrolik disease, Waardenburg syndrome, Warburg Sjo Fledelius Syndrome, Wolf-Hirschhorn syndrome, Wolff Periodic disease, Weissenbacher-Zweymuller syndrome and Xeroderma pigmentosum, among others.

In certain embodiments, a method is provided for treating multiple myeloma comprising administering to a patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition. In another embodiment, a compound of Formula I or Formula II, or a pharmaceutically acceptable salt, isotopic analog, or prodrug thereof, optionally in a pharmaceutically acceptable carrier to form a composition, for use in a method of treating multiple myeloma, wherein the method comprises administering the compound to a patient.

In certain embodiments, a method is provided for managing the progression of multiple myeloma comprising administering to a patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition. In another embodiment, a compound of Formula I, Formula II, Formula III, Formula IV, Formula XV, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition, for use in a method of managing the progression of multiple myeloma, wherein the method comprises administering the compound to a patient.

In certain embodiments, a method is provided for inducing a therapeutic response as assessed by the International Uniform Response Criteria (IURC) for Multiple Myeloma (described in Durie B. G. M; et al. “International uniform response criteria for multiple myeloma. Leukemia 2006, 10(10):1-7) in a patient having multiple myeloma comprising administering to the patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition.

In certain embodiments, a method is provided for treating a solid tumor, for example non-small cell lung cancer or melanoma comprising administering to a patient an effective amount of a compound of the present invention, or a pharmaceutically acceptable salt, isotopic analog, or prodrug thereof, optionally in a pharmaceutically acceptable carrier to form a composition. In another embodiment, a compound of Formula I, or a pharmaceutically acceptable salt, isotopic analog, or prodrug thereof, optionally in a pharmaceutically acceptable carrier to form a composition, for use in a method of treating a solid tumor, for example non-small cell lung cancer or melanoma, wherein the method comprises administering the compound to a patient.

In certain embodiments, a method is provided for managing the progression of multiple myeloma comprising administering to a patient an effective amount of a compound of the present invention, or a pharmaceutically acceptable salt, isotopic analog, or prodrug thereof, optionally in a pharmaceutically acceptable carrier to form a composition. In another embodiment, a compound of Formula I, or a pharmaceutically acceptable salt, isotopic analog, or prodrug thereof, optionally in a pharmaceutically acceptable carrier to form a composition, for use in a method of managing the progression of multiple myeloma, wherein the method comprises administering the compound to a patient.

In certain embodiments the solid tumor is resistant to treatment with an anti PD-1 agent.

In certain embodiments the solid tumor is refractory to treatment with an anti PD-1 agent.

In certain embodiments the solid tumor is resistant to treatment with an anti PD-L1 agent.

In certain embodiments the solid tumor is refractory to treatment with an anti PD-L1 agent.

In another embodiment, a method is provided to achieve a stringent complete response, complete response, or very good partial response, as assessed by the IURC for Multiple Myeloma in a patient having multiple myeloma comprising administering to the patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition.

In another embodiment, a method is provided to achieve an increase in overall survival, progression-free survival, event-free survival, time to process, or disease-free survival in a patient having multiple myeloma comprising administering to the patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition.

In another embodiment, a method is provided to achieve an increase in overall survival in a patient having multiple myeloma comprising administering to the patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition.

In another embodiment, a method is provided to achieve an increase in progression-free survival in a patient having multiple myeloma comprising administering to the patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition.

In another embodiment, a method is provided to achieve an increase in event-free survival in a patient having multiple myeloma comprising administering to the patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition.

In another embodiment, a method is provided to achieve an increase in time to progression in a patient having multiple myeloma comprising administering to the patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition.

In another embodiment, a method is provided to achieve an increase in disease-free survival in a patient having multiple myeloma comprising administering to the patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition.

Methods are also provided to treat patients who have been previously treated for multiple myeloma but are non-responsive to standard therapies in addition to those who have not been previously treated. Additional methods are provided to treat patients who have undergone surgery in an attempt to treat multiple myeloma in addition to those who have not undergone surgery. Methods are also provided to treat patients who have previously undergone transplant therapy in addition to those who have not.

The compounds described herein may be used in the treatment or management of multiple myeloma that is relapsed, refractory, or resistant. In some embodiments, the multiple myeloma is primary, secondary, tertiary, quadruply or quintuply relapsed. In certain embodiments, the compounds described herein may be used to reduce, maintain, or eliminate minimal residual disease (MRD).

The types of multiple myeloma that may be treated with the compounds described herein include, but are not limited to: monoclonal gammopathy of undetermined significance (MGUS); low risk, intermediate risk, or high risk multiple myeloma; newly diagnosed multiple myeloma, including low risk, intermediate risk, or high risk newly diagnosed multiple myeloma); transplant eligible and transplant ineligible multiple myeloma; smoldering (indolent) multiple myeloma (including low risk, intermediate risk, or high risk smoldering multiple myeloma); active multiple myeloma; solitary plasmocytoma; plasma cell leukemia; central nervous system multiple myeloma; light chain myeloma; non-secretory myeloma; Immunoglobulin D myeloma; and Immunoglobulin E myeloma.

In some embodiments, the compounds described herein may be used in the treatment or management of multiple myeloma characterized by genetic abnormalities, for example but not limited to: Cyclin D translocations (for example, t(11; 14)(q13; q32); t(6; 14)(p21; 32); t(12; 14)(p13; q32); or t(6; 20)); MMSET translocations (for example t(4; 14)(p16; q32); MAF translocations (for example t(14; 16)(q32;a32); t(20; 22); t(16; 22)(q11; q13); or t(14; 20)(q32; q11); or other chromosome factors (for example deletion of 17p13 or chromosome 13; del(17/17p), nonhyperdiploidy, and gain (1q)).

In certain embodiments, a method is provided for treating or managing multiple myeloma comprising administering to a patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition, as induction therapy.

In certain embodiments, a method is provided for treating or managing multiple myeloma comprising administering to a patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition, as consolidation therapy.

In certain embodiments, a method is provided for treating or managing multiple myeloma comprising administering to a patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition, as maintenance therapy.

In certain embodiments, the multiple myeloma is plasma cell leukemia.

In certain embodiments, the multiple myeloma is high risk multiple myeloma. In some embodiments, the high risk multiple myeloma is relapsed or refractory. In certain embodiments, the high risk multiple myeloma has relapsed within 12 months of the first treatment. In another embodiment, the high risk multiple myeloma is characterized by genetic abnormalities, for example, one or more of del(17/17p) and t(14; 16)(q32; q32). In some embodiments, the high risk multiple myeloma is relapsed or refractory to one, two or three previous treatments.

In certain embodiments, the multiple myeloma has a p53 mutation. In certain embodiments, the p53 mutation is a Q331 mutation. In certain embodiments, the p53 mutation is a R273H mutation. In certain embodiments, the p53 mutation is a K132 mutation. In certain embodiments, the p53 mutation is a K132N mutation. In certain embodiments, the p53 mutation is a R337 mutation. In certain embodiments, the p53 mutation is a R337L mutation. In certain embodiments, the p53 mutation is a W146 mutation. In certain embodiments, the p53 mutation is a S261 mutation. In certain embodiments, the p53 mutation is a S261T mutation. In certain embodiments, the p53 mutation is a E286 mutation. In certain embodiments, the p53 mutation is a E286K mutation. In certain embodiments, the p53 mutation is a R175 mutation. In certain embodiments, the p53 mutation is a R175H mutation. In certain embodiments, the p53 mutation is a E258 mutation. In certain embodiments, the p53 mutation is a E258K mutation. In certain embodiments, the p53 mutation is a A161 mutation. In certain embodiments, the p53 mutation is a A161T mutation.

In certain embodiments, the multiple myeloma has a homozygous deletion of p53. In certain embodiments, the multiple myeloma has a homozygous deletion of wild-type p53. In certain embodiments, the multiple myeloma has wild-type p53.

In certain embodiments, the multiple myeloma shows activation of one or more oncogenic drivers. In certain embodiments, the one or more oncogenic drivers are selected from the group consisting of C-MAF, MAFB, FGFR3, MMset, Cyclin D1, and Cyclin D. In certain embodiments, the multiple myeloma shows activation of C-MAF. In certain embodiments, the multiple myeloma shows activation of MAFB. In certain embodiments, the multiple myeloma shows activation of FGFR3 and MMset. In certain embodiments, the multiple myeloma shows activation of C-MAF, FGFR3, and MMset. In certain embodiments, the multiple myeloma shows activation of Cyclin D1. In certain embodiments, the multiple myeloma shows activation of MAFB and Cyclin D1. In certain embodiments, the multiple myeloma shows activation of Cyclin D.

In certain embodiments, the multiple myeloma has one or more chromosomal translocations. In certain embodiments, the chromosomal translocation is t(14; 16). In certain embodiments, the chromosomal translocation is t(14; 20). In certain embodiments, the chromosomal translocation is t(4; 14). In certain embodiments, the chromosomal translocations are t(4; 14) and t(14; 16). In certain embodiments, the chromosomal translocation is t(11; 14). In certain embodiments, the chromosomal translocation is t(6; 20). In certain embodiments, the chromosomal translocation is t(20; 22). In certain embodiments, the chromosomal translocations are t(6; 20) and t(20; 22). In certain embodiments, the chromosomal translocation is t(16; 22). In certain embodiments, the chromosomal translocations are t(14; 16) and t(16; 22). In certain embodiments, the chromosomal translocations are t(14; 20) and t(11; 14).

In certain embodiments, the multiple myeloma has a Q331 p53 mutation, activation of C-MAF, and a chromosomal translocation at t(14; 16). In certain embodiments, the multiple myeloma has homozygous deletion of p53, activation of C-MAF, and a chromosomal translocation at t(14; 16). In certain embodiments, the multiple myeloma has a K132N p53 mutation, activation of MAFB, and a chromosomal translocation at t(14; 20). In certain embodiments, the multiple myeloma has wild type p53, activation of FGFR3 and MMset, and a chromosomal translocation at t(4; 14). In certain embodiments, the multiple myeloma has wild type p53, activation of C-MAF, and a chromosomal translocation at t(14; 16). In certain embodiments, the multiple myeloma has homozygous deletion of p53, activation of FGFR3, MMset, and C-MAF, and chromosomal translocations at t(4; 14) and t(14; 16). In certain embodiments, the multiple myeloma has homozygous deletion of p53, activation of Cyclin D1, and a chromosomal translocation at t(11; 14). In certain embodiments, the multiple myeloma has a R337L p53 mutation, activation of Cyclin D1, and a chromosomal translocation at t(11; 14). In certain embodiments, the multiple myeloma has a W146 p53 mutation, activation of FGFR3 and MMset, and a chromosomal translocation at t(4; 14). In certain embodiments, the multiple myeloma has a S261T p53 mutation, activation of MAFB, and chromosomal translocations at t(6; 20) and t(20; 22). In certain embodiments, the multiple myeloma has a E286K p53 mutation, by activation of FGFR3 and MMset, and a chromosomal translocation at t(4; 14). In certain embodiments, the multiple myeloma has a R175H p53 mutation, activation of FGFR3 and MMset, and a chromosomal translocation at t(4; 14). In certain embodiments, the multiple myeloma has a E258K p53 mutation, activation of C-MAF, and chromosomal translocations at t(14; 16) and t(16; 22). In certain embodiments, the multiple myeloma has wild type p53, activation of MAFB and Cyclin D1, and chromosomal translocations at t(14; 20) and t(11; 14). In certain embodiments, the multiple myeloma has a A161T p53 mutation, activation of Cyclin D, and a chromosomal translocation at t(11; 14).

In some embodiments, the multiple myeloma is transplant eligible newly diagnosed multiple myeloma. In other embodiments, the multiple myeloma is transplant ineligible newly diagnosed multiple myeloma.

In some embodiments, the multiple myeloma shows early progression (for example less than 12 months) following initial treatment. In other embodiments, the multiple myeloma shows early progression (for example less than 12 months) following autologous stem cell transplant. In another embodiment, the multiple myeloma is refractory to lenalidomide. In another embodiment, the multiple myeloma is refractory to pomalidomide. In some such embodiments, the multiple myeloma is predicted to be refractory to pomalidomide (for example, by molecular characterization). In another embodiment, the multiple myeloma is relapsed or refractory to 3 or more treatments and was exposed to a proteasome inhibitor (for example, bortezomib, carfilzomib, ixazomib, oprozomib, or marizomib) and an immunomodulatory compound (for example thalidomide, lenalidomide, pomalidomide, iberdomide, or avadomide), or double refractory to a proteasome inhibitor and an immunomodulatory compound. In still other embodiments, the multiple myeloma is relapsed or refractory to 3 or more prior therapies, including for example, a CD38 monoclonal antibody (CD38 mAb, for example, daratumumab orisatuximab), a proteasome inhibitor (for example, bortezomib, carfilzomib, ixazomib, or marizomib), and an immunomodulatory compound (for example thalidomide, lenalidomide, pomalidomide, iberdomide, or avadomide) or double refractory to a proteasome inhibitor or immunomodulatory compound and a CD38 mAb. In still other embodiments, the multiple myeloma is triple refractory, for example, the multiple myeloma is refractory to a proteasome inhibitor (for example, bortezomib, carfilzomib, ixazomib, oprozomib or marizomib), an immunomodulatory compound (for example thalidomide, lenalidomide, pomalidomide, iberdomide, or avadomide), and one other active agent, as described herein.

In certain embodiments, a method is provided for treating or managing relapsed or refractory multiple myeloma in patients with impaired renal function or a symptom thereof comprising administering to a patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition.

In another embodiment, a method is provided for treating or managing relapsed or refractory multiple myeloma in frail patients comprising administering to a patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition, wherein the frail patient is characterized by ineligibility for induction therapy or intolerance to dexamethasone treatment. In other embodiments, the frail patient is elderly, for example, older than 65 years old.

In another embodiment, a method is provided for treating or managing fourth line relapsed or refractory multiple myeloma comprising administering to a patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition.

In another embodiment, a method is provided for treating or managing newly diagnosed, transplant-ineligible multiple myeloma comprising administering to a patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition.

In another embodiment, a method is provided for treating or managing newly diagnosed, transplant-ineligible multiple myeloma comprising administering to a patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition, as maintenance therapy after another therapy or transplant.

In another embodiment, a method is provided for treating or managing high risk multiple myeloma that is relapsed or refractory to one, two, or three previous treatments comprising administering to a patient an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition.

In some embodiments, the patient to be treated by one of the compounds described herein has not be treated with multiple myeloma therapy prior to administration. In some embodiments, the patient to be treated by one of the compounds described herein has been treated by multiple myeloma therapy prior to administration. In some embodiments, the patient to be treated by one of the compounds described herein has developed drug resistant to the multiple myeloma therapy. In some embodiments, the patient to be treated by one of the compounds described herein has developed resistance to one, two, or three multiple myeloma therapies, wherein the therapies are selected from a CD38 antibody (CD38 mAB, for example, daratumumab or isatuximab), a proteasome inhibitor (for example, bortezomib, carfilzomib, ixazomib, or marizomib), and an immunomodulatory compound (for example thalidomide, lenalidomide, pomalidomide, iberdomide, or avodomide).

In certain embodiments an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition is administered to treat a patient with a viral infection.

The viral infection to be treated or prevented can be caused by any virus, including but not limited to, “African Swine Fever Viruses,” Arbovirus, Adenoviridae, Arenaviridae, Arterivirus, Astroviridae, Baculoviridae, Bimaviridae, Birnaviridae, Bunyaviridae, Caliciviridae, Caulimoviridae, Circoviridae, Coronaviridae, Cystoviridae, Dengue, EBV, HIV, Deltaviridae, Filviridae, Filoviridae, Flaviviridae, Hepadnaviridae (Hepatitis), Herpesviridae (such as, Cytomegalovirus, Herpes Simplex, Herpes Zoster), Iridoviridae, Mononegavirus (e.g., Paramyxoviridae, Morbillivirus, Rhabdoviridae), Myoviridae, Orthomyxoviridae (e.g., Influenza A, Influenza B, and parainfluenza), Papiloma virus, Papovaviridae, Paramyxoviridae, Prions, Parvoviridae, Phycodnaviridae, Picomaviridae (e.g. Rhinovirus, Poliovirus), Poxviridae (such as Smallpox or Vaccinia), Potyviridae, Reoviridae (e.g., Rotavirus), Retroviridae (HTLV-I, HTLV-II, Lentivirus), Rhabdoviridae, Tectiviridae, Togaviridae (e.g., Rubivirus), or any combination thereof. In another embodiment of the invention, the viral infection is caused by a virus selected from the group consisting of herpes, pox, papilloma, corona, influenza, hepatitis, sendai, sindbis, vaccinia viruses, west nile, hanta, or viruses which cause the common cold. In another embodiment of the invention, the condition to be treated is selected from the group consisting of AIDS, viral meningitis, Dengue, EBV, hepatitis, and any combination thereof.

In certain embodiments, the viral infection is, but is not limited to, Coronavirus, SARS-CoV1, SARS-CoV2, MERS, HIV, HBV, HCV, RSV, HPV, HSV, CMV, flavivirus, pestivirus, coronavirus, noroviridae, rhinovirus, Ebola, Rotavirus, Influenza, EBV, viral pneumonia, drug-resistant viruses, Bird flu, RNA virus, DNA virus, adenovirus, poxvirus, Picornavirus, Togavirus, Orthomyxovirus, Retrovirus, Epstein-Barr virus (EBV)+ or Hepadnovirus.

In certain embodiments, the viral infection including but not limited to HIV, HBV, HCV or RSV.

In certain embodiments an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition is administered to treat a patient with a fungal infection. By modulating a patient's immune system response, a compound of the present invention can either treat a fungal infection on its own or be used in combination with an additional active agent.

Non-limiting examples of fungal infections include athlete's foot, jock itch, ringworm, yeast infection, onychomycosis, fungal infection of the nail, and fungal infection of the skin.

In certain embodiments an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, isotopic analog, or prodrug thereof, optionally in a pharmaceutically acceptable carrier to form a composition is administered to treat a patient with a bacterial infection. By modulating a patient's immune system response, a compound of the present invention can either treat a bacterial infection on its own or be used in combination with an additional active agent.

Non-limiting examples of bacterial infections include strep throat, bacterial urinary tract infection, coliform bacterial infection, bacterial food poisoning, E. Coli, Salmonella, Shigella, bacterial cellulitis, Staphylococcus Aureus, bacterial vaginosis, gonorrhea, chlamydia, syphilis, Clostridium Difficile, tuberculosis, whooping cough, pneumococcal pneumonia, bacterial meningitis, Lyme disease, cholera, botulism, tetanus, and anthrax.

The compounds described herein can be used to treat a patient regardless of patient's age. In some embodiments, the subject is 18 years or older. In other embodiments, the subject is more than 18, 25, 35, 40, 45, 50, 55, 60, 65, or 70 years old. In other embodiments, the patient is less than 65 years old. In other embodiments, the patient is more than 65 years old. In certain embodiments, the patient is an elderly multiple myeloma patient, such as a patient older than 65 years old. In certain embodiments, the patient is an elderly multiple myeloma patient, such as a patient older than 75 years old.

In certain embodiments the compound of the present invention forms a neomorphic surface that provides a binding site for the protein of interest, a chaperone, a complex sub-unit, or a binding partner, which ultimately leads to the protein of interests degradation and/or codegradation.

### IV. Combination Therapy

Any of the compounds described herein can be used in an effective amount alone or in combination to treat a host such as a human with a disorder as described herein.

The disclosed compounds described herein can be used in an effective amount alone or in combination with another compound of the present invention or another bioactive agent or second therapeutic agent to treat a patient such as a human with a disorder, including but not limited to those described herein.

The term “bioactive agent” or “additional therapeutically active agent” is used to describe an agent, other than the compound according to the present invention, which can be used in combination or alternation with a compound of the present invention to achieve a desired result of therapy. In certain embodiments, the compound of the present invention and the additional therapeutically active agent are administered in a manner that they are active in vivo during overlapping time periods, for example, have time-period overlapping Cmax, Tmax, AUC or other pharmacokinetic parameter. In another embodiment, the compound of the present invention and the additional therapeutically active agent are administered to a host in need thereof that do not have overlapping pharmacokinetic parameter, however, one has a therapeutic impact on the therapeutic efficacy of the other.

In one aspect of this embodiment, the additional therapeutically active agent is an immune modulator, including but not limited to a checkpoint inhibitor, including as non-limiting examples, a PD-1 inhibitor, PD-L1 inhibitor, PD-L2 inhibitor, CTLA-4 inhibitor, LAG-3 inhibitor, TIM-3 inhibitor, V-domain Ig suppressor of T-cell activation (VISTA) inhibitors, small molecule, peptide, nucleotide, or other inhibitor. In certain aspects, the immune modulator is an antibody, such as a monoclonal antibody.

PD-1 inhibitors that blocks the interaction of PD-1 and PD-L1 by binding to the PD-1 receptor, and in turn inhibit immune suppression include, for example, nivolumab (Opdivo), pembrolizumab (Keytruda), pidilizumab, AMP-224 (AstraZeneca and MedImmune), PF-06801591 (Pfizer), MEDIO680 (AstraZeneca), PDR001 (Novartis), REGN2810 (Regeneron), SHR-12-1 (Jiangsu Hengrui Medicine Company and Incyte Corporation), TSR-042 (Tesaro), and the PD-L1/VISTA inhibitor CA-170 (Curis Inc.). PD-L1 inhibitors that block the interaction of PD-1 and PD-L1 by binding to the PD-L1 receptor, and in turn inhibits immune suppression, include for example, atezolizumab (Tecentriq), durvalumab (AstraZeneca and MedImmune), KN035 (Alphamab), and BMS-936559 (Bristol-Myers Squibb). CTLA-4 checkpoint inhibitors that bind to CTLA-4 and inhibits immune suppression include, but are not limited to, ipilimumab, tremelimumab (AstraZeneca and MedImmune), AGEN1884 and AGEN2041 (Agenus). LAG-3 checkpoint inhibitors, include, but are not limited to, BMS-986016 (Bristol-Myers Squibb), GSK2831781 (GlaxoSmithKline), IMP321 (Prima BioMed), LAG525 (Novartis), and the dual PD-1 and LAG-3 inhibitor MGD013 (MacroGenics). An example of a TIM-3 inhibitor is TSR-022 (Tesaro).

In certain embodiments the checkpoint inhibitor is selected from nivolumab/OPDIVO®; pembrolizumab/KEYTRUDA®; and pidilizumab/CT-011, MPDL3280A/RG7446; MEDI4736; MSB0010718C; BMS 936559, a PDL2/1 g fusion protein such as AMP 224 or an inhibitor of B7-H3 (e.g., MGA271), B7-H4, BTLA, HVEM, TIM3, GAL9, LAG 3, VISTA, KIR, 2B4, CD160, CGEN-15049, CHK 1, CHK2, A2aR, B-7 family ligands, or a combination thereof.

In certain embodiments, the PD-1 inhibitor is BGB-A317. In certain embodiments, the PD-L1 inhibitor is MED14736. In certain embodiments, the PD-L2 inhibitor is rHIgM12B7A.

In certain embodiments, the checkpoint inhibitor is a B7 inhibitor, for example a B7-H3 inhibitor or a B7-H4 inhibitor. In certain embodiments, the B7-H3 inhibitor is MGA271.

In certain embodiments, the checkpoint inhibitor is an OX40 agonist. In certain embodiments, the checkpoint inhibitor is an anti-OX40 antibody, for example anti-OX-40 or MEDI6469.

In certain embodiments, the checkpoint inhibitor is a GITR agonist. In certain embodiments, the GITR agonist is an anti-GITR antibody, for example TRX518.

In certain embodiments, the checkpoint inhibitor is a CD137 agonist. In certain embodiments, the CD137 agonist is an anti-CD137 antibody, for example PF-05082566.

In certain embodiments, the checkpoint inhibitor is a CD40 agonist. In certain embodiments, the CD40 agonist is an anti-CD40 antibody, for example CF-870,893.

In certain embodiments, the checkpoint inhibitor is an IDO inhibitor, for example INCB24360 or indoximod.

In another embodiment, an active compounds described herein can be administered in an effective amount for the treatment of abnormal tissue of the male reproductive system such as prostate or testicular cancer, in combination or alternation with an effective amount of an androgen (such as testosterone) inhibitor including but not limited to a selective androgen receptor modulator, a selective androgen receptor degrader, a complete androgen receptor degrader, or another form of partial or complete androgen antagonist. In certain embodiments, the prostate or testicular cancer is androgen-resistant. Non-limiting examples of anti-androgen compounds are provided in WO 2011/156518 and U.S. Pat. Nos. 8,455,534 and 8,299,112. Additional non-limiting examples of anti-androgen compounds include: enzalutamide, apalutamide, cyproterone acetate, chlormadinone acetate, spironolactone, canrenone, drospirenone, ketoconazole, topilutamide, abiraterone acetate, and cimetidine.

In certain embodiments, the additional therapeutically active agent is an ALK inhibitor. Examples of ALK inhibitors include but are not limited to Crizotinib, Alectinib, ceritinib, TAE684 (NVP-TAE684), GSK1838705A, AZD3463, ASP3026, PF-06463922, entrectinib (RXDX-101), and AP26113.

In certain embodiments, the additional therapeutically active agent is an EGFR inhibitor. Examples of EGFR inhibitors include erlotinib (Tarceva), gefitinib (Iressa), afatinib (Gilotrif), rociletinib (CO-1686), osimertinib (Tagrisso), olmutinib (Olita), naquotinib (ASP8273), nazartinib (EGF816), PF-06747775 (Pfizer), icotinib (BPI-2009), neratinib (HKI-272; PB272); avitinib (AC0010), EAI045, tarloxotinib (TH-4000; PR-610), PF-06459988 (Pfizer), tesevatinib (XL647; EXEL-7647; KD-019), transtinib, WZ-3146, WZ8040, CNX-2006, and dacomitinib (PF-00299804; Pfizer).

In certain embodiments, the additional therapeutically active agent is an HER-2 inhibitor. Examples of HER-2 inhibitors include trastuzumab, lapatinib, ado-trastuzumab emtansine, and pertuzumab.

In certain embodiments, the additional therapeutically active agent is a CD20 inhibitor. Examples of CD20 inhibitors include obinutuzumab, rituximab, fatumumab, ibritumomab, tositumomab, and ocrelizumab.

In certain embodiments, the additional therapeutically active agent is a JAK3 inhibitor. Examples of JAK3 inhibitors include tasocitinib.

In certain embodiments, the additional therapeutically active agent is a BCL-2 inhibitor. Examples of BCL-2 inhibitors include venetoclax, ABT-199 (4-[4-[[2-(4-Chlorophenyl)-4,4-dimethylcyclohex-1-en-1-yl]methyl]piperazin-1-yl]-N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-737 (4-[4-[[2-(4-chlorophenyl)phenyl]methyl]piperazin-1-yl]-N-[4-[[(2R)-4-(dimethylamino)-1-phenylsulfanylbutan-2-yl] amino]-3-nitrophenyl]sulfonylbenzamide) (navitoclax), ABT-263 ((R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3((trifluoromethyl)sulfonyl)phenyl)sulfonyl)benzamide), GX15-070 (obatoclax mesylate, (2Z)-2-[(5Z)-5-[(3,5-dimethyl-1H-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-Dimethylethyl)phenyl]sulfonyl]phenyl]-2,3,4-trihydroxy-5-[[2-(1-methylethyl)phenyl]methyl]benzamide), Apogossypolone (ApoG2), HA14-1, AT101, sabutoclax, gambogic acid, or G3139 (Oblimersen).

In certain embodiments, the additional therapeutically active agent is a kinase inhibitor. In certain embodiments, the kinase inhibitor is selected from a phosphoinositide 3-kinase (PI3K) inhibitor, a Bruton's tyrosine kinase (BTK) inhibitor, or a spleen tyrosine kinase (Syk) inhibitor, or a combination thereof.

Examples of PI3 kinase inhibitors include but are not limited to Wortmannin, demethoxyviridin, perifosine, idelalisib, Pictilisib, Palomid 529, ZSTK474, PWT33597, CUDC-907, and AEZS-136, duvelisib, GS-9820, BKM120, GDC-0032 (Taselisib) (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-1-phenoxy-2-butanyl]oxy}phosphonium)), BYL-719 ((2S)-N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide) (omipalisib), TGX-221 ((±)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 ((R)-2-((1-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[1-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), Buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methylsulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N′-[4-(4,6-di-4-morpholinyl-1,3,5-triazin-2-yl)phenyl]urea) (gedatolisib), LY3023414, BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile) (dactolisib), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5h]isochromen-10-yl] acetate (also known as sonolisib)), LY294002, AZD8186, PF-4989216, pilaralisib, GNE-317, PI-3065, PI-103, NU7441 (KU-57788), HS 173, VS-5584 (SB2343), CZC24832, TG100-115, A66, YM201636, CAY10505, PIK-75, PIK-93, AS-605240, BGT226 (NVP-BGT226), AZD6482, voxtalisib, alpelisib, IC-87114, TGI100713, CH5132799, PKI-402, copanlisib (BAY 80-6946), XL 147, PIK-90, PIK-293, PIK-294, 3-MA (3-methyladenine), AS-252424, AS-604850, apitolisib (GDC-0980; RG7422), and the structure described in WO2014/071109.

Examples of BTK inhibitors include ibrutinib (also known as PCI-32765)(Imbruvica™)(1-[(3R)-3-[4-amino-3-(4-phenoxy-phenyl)pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (see US Patent Publication No 2011/0117073, incorporated herein in its entirety), Dasatinib ([N-(2-chloro-6-methylphenyl)-2-(6-(4-(2-hydroxyethyl)piperazin-1-yl)-2-methylpyrimidin-4-ylamino)thiazole-5-carboxamide], LFM-A13 (alpha-cyano-beta-hydroxy-beta-methyl-N-(2,5-ibromophenyl) propenamide), GDC-0834 ([R-N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino)phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)-N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)-N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(1H)-one), and RN486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one), and other molecules capable of inhibiting BTK activity, for example those BTK inhibitors disclosed in Akinleye et ah, Journal of Hematology & Oncology, 2013, 6:59, the entirety of which is incorporated herein by reference.

Syk inhibitors include, for example, Cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), R09021 (6-[(1R,2S)-2-Amino-cyclohexylamino]-4-(5,6-dimethyl-pyridin-2-ylamino)-pyridazine-3-carboxylic acid amide), imatinib (Gleevac; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), piceatannol (3-Hydroxyresveratol), YM193306(see Singh et al. Discovery and Development of Spleen Tyrosine Kinase (SYK) Inhibitors, J. Med. Chem. 2012, 55, 3614-3643), 7-azaindole, piceatannol, ER-27319 (see Singh et al. Discovery and Development of Spleen Tyrosine Kinase (SYK) Inhibitors, J. Med. Chem. 2012, 55, 3614-3643 incorporated in its entirety herein), Compound D (see Singh et al. Discovery and Development of Spleen Tyrosine Kinase (SYK) Inhibitors, J. Med. Chem. 2012, 55, 3614-3643 incorporated in its entirety herein), PRT060318 (see Singh et al. Discovery and Development of Spleen Tyrosine Kinase (SYK) Inhibitors, J. Med. Chem. 2012, 55, 3614-3643 incorporated in its entirety herein), luteolin (see Singh et al. Discovery and Development of Spleen Tyrosine Kinase (SYK) Inhibitors, J. Med. Chem. 2012, 55, 3614-3643 incorporated in its entirety herein), apigenin (see Singh et al. Discovery and Development of Spleen Tyrosine Kinase (SYK) Inhibitors, J. Med. Chem. 2012, 55, 3614-3643 incorporated in its entirety herein), quercetin (see Singh et al. Discovery and Development of Spleen Tyrosine Kinase (SYK) Inhibitors, J. Med. Chem. 2012, 55, 3614-3643 incorporated in its entirety herein), fisetin (see Singh et al. Discovery and Development of Spleen Tyrosine Kinase (SYK) Inhibitors, J. Med. Chem. 2012, 55, 3614-3643 incorporated in its entirety herein), myricetin (see Singh et al. Discovery and Development of Spleen Tyrosine Kinase (SYK) Inhibitors, J. Med. Chem. 2012, 55, 3614-3643 incorporated in its entirety herein), morin (see Singh et al. Discovery and Development of Spleen Tyrosine Kinase (SYK) Inhibitors, J. Med. Chem. 2012, 55, 3614-3643 incorporated in its entirety herein).

In certain embodiments, the additional therapeutically active agent is a MEK inhibitor. MEK inhibitors are well known, and include, for example, trametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin−1(2H-yl}phenyl)acetamide), selumetinib (6-(4-bromo-2-chloroanilino)-7-fluoro-N-(2-hydroxyethoxy)-3-methylbenzimidazole-5-carboxamide), pimasertib/AS703026/MSC 1935369 ((S)-N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol), refametinib/BAY869766/RDEA1 19 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3-d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6-carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2yl)methyl)benzamide), or AZD8330 (2-((2-fluoro-4-iodophenyl)amino)-N-(2 hydroxyethoxy)-1,5-dimethyl-6-oxo-1,6-dihydropyridine-3-carboxamide), U0126-EtOH, PD184352 (CI-1040), GDC-0623, BI-847325, cobimetinib, PD98059, BIX 02189, BIX 02188, binimetinib, SL-327, TAK-733, PD318088.

In certain embodiments, the additional therapeutically active agent is a Raf inhibitor. Raf inhibitors are known and include, for example, Vemurafinib (N-[3-[[5-(4-Chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carboxamide; 4-methylbenzenesulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)-N-(3-(trifluoromethyl)phenyl)benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-Bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), Sorafenib N-Oxide (4-[4-[[[[4-Chloro-3(trifluoroMethyl)phenyl]aMino]carbonyl]aMino]phenoxy]-N-Methyl-2pyridinecarboxaMide 1-Oxide), PLX-4720, dabrafenib (GSK2118436), GDC-0879, RAF265, AZ 628, Sf590885, ZM336372, GW5074, TAK-632, CEP-32496, LY3009120, and GX818 (Encorafenib).

In certain embodiments, the additional therapeutically active agent is an AKT inhibitor, including but not limited to, MK-2206, GSK690693, Perifosine, (KRX-0401), GDC-0068, Triciribine, AZD5363, Honokiol, PF-04691502, and Miltefosine, a FLT-3 inhibitor, including but not limited to, P406, Dovitinib, Quizartinib (AC220), Amuvatinib (MP-470), Tandutinib (MLN518), ENMD-2076, and KW-2449, or a combination thereof.

In certain embodiments, the additional therapeutically active agent is an mTOR inhibitor. Examples of mTOR inhibitors include but are not limited to rapamycin and its analogs, everolimus (Afinitor), temsirolimus, ridaforolimus, sirolimus, and deforolimus. Examples of MEK inhibitors include but are not limited to tametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin−1(2H-yl}phenyl)acetamide), selumetinob (6-(4-bromo-2-chloroanilino)-7-fluoro-N-(2-hydroxyethoxy)-3-methylbenzimidazole-5-carboxamide), pimasertib/AS703026/MSC1935369 ((S)-N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol) (cobimetinib), refametinib/BAY869766/RDEA119 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6 carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2 yl)methyl)benzamide), or AZD8330 (2-((2-fluoro-4-iodophenyl)amino)-N-(2-hydroxyethoxy)-1,5-dimethyl-6-oxo-1,6-dihydropyridine-3-carboxamide).

In certain embodiments, the additional therapeutically active agent is a RAS inhibitor. Examples of RAS inhibitors include but are not limited to Reolysin and siG12D LODER.

In certain embodiments, the additional therapeutically active agent is a HSP inhibitor. HSP inhibitors include but are not limited to Geldanamycin or 17-N-Allylamino-17-demethoxygeldanamycin (17AAG), and Radicicol.

Additional bioactive compounds include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an aurora kinase inhibitor, a PIK-1 modulator, an HDAC inhbitor, a c-MET inhibitor, a PARP inhibitor, a Cdk inhibitor, an IGFR-TK inhibitor, an anti-HGF antibody, a focal adhesion kinase inhibitor, a Map kinase kinase (mek) inhibitor, a VEGF trap antibody, pemetrexed, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

In certain embodiments, the additional therapeutically active agent is selected from, but are not limited to, Imatinib mesylate (Gleevac®), Dasatinib (Sprycel®), Nilotinib (Tasigna®), Bosutinib (Bosulif®), Trastuzumab (Herceptin®), trastuzumab-DM1, Pertuzumab (Perjeta™), Lapatinib (Tykerb®), Gefitinib (Iressa®), Erlotinib (Tarceva®), Cetuximab (Erbitux®), Panitumumab (Vectibix®), Vandetanib (Caprelsa®), Vemurafenib (Zelboraf®), Vorinostat (Zolinza®), Romidepsin (Istodax®), Bexarotene (Tagretin®), Alitretinoin (Panretin®), Tretinoin (Vesanoid®), Carfilizomib (Kyprolis™), Pralatrexate (Folotyn®), Bevacizumab (Avastin®), Ziv-aflibercept (Zaltrap®), Sorafenib (Nexavar®), Sunitinib (Sutent®), Pazopanib (Votrient®), Regorafenib (Stivarga®), and Cabozantinib (Cometriq™).

In certain aspects, the additional therapeutically active agent is an anti-inflammatory agent, a chemotherapeutic agent, a radiotherapeutic, an additional therapeutic agent, or an immunosuppressive agent.

Suitable chemotherapeutic additional therapeutically active agent s include, but are not limited to, a radioactive molecule, a toxin, also referred to as cytotoxin or cytotoxic agent, which includes any agent that is detrimental to the viability of cells, and liposomes or other vesicles containing chemotherapeutic compounds. General anticancer pharmaceutical agents include: Vincristine (Oncovin®) or liposomal vincristine (Marqibo®), Daunorubicin (daunomycin or Cerubidine®) or doxorubicin (Adriamycin®), Cytarabine (cytosine arabinoside, ara-C, or Cytosar®), L-asparaginase (Elspar®) or PEG-L-asparaginase (pegaspargase or Oncaspar®), Etoposide (VP-16), Teniposide (Vumon®), 6-mercaptopurine (6-MP or Purinethol®), Methotrexate, Cyclophosphamide (Cytoxan®), Prednisone, Dexamethasone (Decadron), imatinib (Gleevec®), dasatinib (Sprycel®), nilotinib (Tasigna®), bosutinib (Bosulif®), and ponatinib (Iclusig™). Examples of additional suitable chemotherapeutic agents include but are not limited to 1-dehydrotestosterone, 5-fluorouracil decarbazine, 6-mercaptopurine, 6-thioguanine, actinomycin D, adriamycin, aldesleukin, an alkylating agent, allopurinol sodium, altretamine, amifostine, anastrozole, anthramycin (AMC)), an anti-mitotic agent, cis-dichlorodiamine platinum (II) (DDP) cisplatin), diamino dichloro platinum, anthracycline, an antibiotic, an antimetabolite, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), Chlorambucil, Cisplatin, Cladribine, Colchicin, conjugated estrogens, Cyclophosphamide, Cyclothosphamide, Cytarabine, Cytarabine, cytochalasin B, Cytoxan, Dacarbazine, Dactinomycin, dactinomycin (formerly actinomycin), daunirubicin HCL, daunorucbicin citrate, denileukin diftitox, Dexrazoxane, Dibromomannitol, dihydroxy anthracin dione, Docetaxel, dolasetron mesylate, doxorubicin HCL, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon α-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

In some embodiments, the compound of the present invention is administered in combination with a chemotherapeutic agent (e.g., a cytotoxic agent or other chemical compound useful in the treatment of cancer). Examples of chemotherapeutic agents include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel, and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Inti. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5- FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, NJ), ABRAXANE®, cremophor-free, albumin-engineered nanoparticle formulation of paclitaxel (American Pharmaceutical Partners, Schaumberg, IL), and TAXOTERE® doxetaxel (Rhone-Poulene Rorer, Antony, France); chloranbucil; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the compound of the present invention. Suitable dosing regimens of combination chemotherapies are known in the ar. For example combination dosing regimes are described in Saltz et al., Proc. Am. Soc. Clin. Oncol. 18:233a (1999) and Douillard et al., Lancet 355(9209): 1041-1047 (2000).

Additional therapeutic agents that can be administered in combination with a Degrader disclosed herein can include bevacizumab, sutinib, sorafenib, 2-methoxyestradiol or 2ME2, finasunate, vatalanib, vandetanib, aflibercept, volociximab, etaracizumab (MEDI-522), cilengitide, erlotinib, cetuximab, panitumumab, gefitinib, trastuzumab, dovitinib, figitumumab, atacicept, rituximab, alemtuzumab, aldesleukine, atlizumab, tocilizumab, temsirolimus, everolimus, lucatumumab, dacetuzumab, HLL1, huN901-DM1, atiprimod, natalizumab, bortezomib, carfilzomib, marizomib, tanespimycin, saquinavir mesylate, ritonavir, nelfinavir mesylate, indinavir sulfate, belinostat, panobinostat, mapatumumab, lexatumumab, dulanermin, ABT-737, oblimersen, plitidepsin, talmapimod, P276-00, enzastaurin, tipifarnib, perifosine, imatinib, dasatinib, lenalidomide, thalidomide, simvastatin, celecoxib, bazedoxifene, AZD4547, rilotumumab, oxaliplatin (Eloxatin), PD0332991, ribociclib (LEE011), amebaciclib (LY2835219), HDM201, fulvestrant (Faslodex), exemestane (Aromasin), PIM447, ruxolitinib (INC424), BGJ398, necitumumab, pemetrexed (Alimta), and ramucirumab (IMC-1121B).

In certain embodiments, the additional therapy is a monoclonal antibody (MAb). Some MAbs stimulate an immune response that destroys cancer cells. Similar to the antibodies produced naturally by B cells, these MAbs may “coat” the cancer cell surface, triggering its destruction by the immune system. For example, bevacizumab targets vascular endothelial growth factor(VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels. Similarly, cetuximab and panitumumab target the epidermal growth factor receptor (EGFR), and trastuzumab targets the human epidermal growth factor receptor 2 (HER-2). MAbs that bind to cell surface growth factor receptors prevent the targeted receptors from sending their normal growth-promoting signals. They may also trigger apoptosis and activate the immune system to destroy tumor cells.

In one aspect of the present invention, the additional therapeutically active agent is an immunosuppressive agent. The immunosuppressive agent can be a calcineurin inhibitor, e.g. a cyclosporin or an ascomycin, e.g. Cyclosporin A (NEORAL®), FK506 (tacrolimus), pimecrolimus, a mTOR inhibitor, e.g. rapamycin or a derivative thereof, e.g. Sirolimus (RAPAMUNE®), Everolimus (Certican®), temsirolimus, zotarolimus, biolimus-7, biolimus-9, a rapalog, e.g. ridaforolimus, azathioprine, campath 1H, a S1P receptor modulator, e.g. fingolimod or an analogue thereof, an anti IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3@), Prednisone, ATGAM®, THYMOGLOBULIN®, Brequinar Sodium, OKT4, T10B9.A-3A, 33B3.1, 15-deoxyspergualin, tresperimus, Leflunomide ARAVA®, CTLAI-Ig, anti-CD25, anti-IL2R, Basiliximab (SIMULECT®), Daclizumab (ZENAPAX®), mizorbine, methotrexate, dexamethasone, ISAtx-247, SDZ ASM 981 (pimecrolimus, Elidel®), CTLA41g (Abatacept), belatacept, LFA31g, etanercept (sold as Enbrel® by Immunex), adalimumab (Humira®), infliximab (Remicade®), an anti-LFA-1 antibody, natalizumab (Antegren®), Enlimomab, gavilimomab, antithymocyte immunoglobulin, siplizumab, Alefacept efalizumab, pentasa, mesalazine, asacol, codeine phosphate, benorylate, fenbufen, naprosyn, diclofenac, etodolac and indomethacin, aspirin and ibuprofen.

In certain embodiments, the additional therapy is bendamustine. In certain embodiments, the additional therapy is obinutuzmab. In certain embodiments, the additional therapy is a proteasome inhibitor, for example ixazomib or oprozomib. In certain embodiments, the additional therapy is a histone deacetylase inhibitor, for example ACY241. In certain embodiments, the additional therapy is a BET inhibitor, for example GSK525762A, OTX015, BMS-986158, TEN-010, CPI-0610, INCB54329, BAY1238097, FT-1101, ABBV-075, BI 894999, GS-5829, GSK1210151A (I-BET-151), CPI-203, RVX-208, XD46, MS436, PFI-1, RVX2135, ZEN3365, XD14, ARV-771, MZ-1, PLX5117, 4-[2-(cyclopropylmethoxy)-5-(methanesulfonyl)phenyl]-2-methylisoquinolin-1(2H)-one, EP11313 and EP11336. In certain embodiments, the additional therapy is an MCL-1 inhibitor, for example AZD5991, AMG176, MIK665, S64315, or S63845. In certain embodiments, the additional therapy is an LSD-1 inhibitor, for example ORY-1001, ORY-2001, INCB-59872, IMG-7289, TAK-418, GSK-2879552, 4-[2-(4-amino-piperidin-1-yl)-5-(3-fluoro-4-methoxy-phenyl)-1-methyl-6-oxo-1,6-dihydropyrimidin-4-yl]-2-fluoro-benzonitrile or a salt thereof. In certain embodiments, the additional therapy is a CS1 antibody, for example elotuzumab. In certain embodiments, the additional therapy is a CD38 antibody, for example daratumumab or isatuximab. In certain embodiments, the additional therapy is a BCMA antibody or antibody-conjugate, for example GSK2857916 or BI 836909.

In some embodiments, a degrader described herein is administered in combination or alternation with one or more cellular immunotherapeutics. In some embodiments, the cellular immunotherapeutic is an engineered immune cell. Engineered immune cells include, for example, but are not limited to, engineered T-cell receptor (TCR) cells and engineered chimeric antigen receptor (CAR) cells. Engineered T Cell Receptor (TCR) Therapy generally involves the introduction of an engineered T cell receptor targeting specific cancer antigens into a patient or donor derived immune effector cell, for example a T-cell or natural killer cell. Alternatively, Chimeric Antigen Receptor (CAR) Therapy generally involves the introduction of a chimeric antigen receptor targeting a specific cancer antigen into a patient or donor derived immune effector cell, for example a T-cell, natural killer cells, or macrophage. One key advantage of CARs compared to TCRs is their ability to bind to cancer cells even if their antigens aren't presented on the surface via MHC, which can render more cancer cells vulnerable to their attacks. However, CAR cells can only recognize antigens that themselves are naturally expressed on the cell surface, so the range of potential antigen targets is smaller than with TCRs.

In some embodiments, the immunotherapeutic is an engineered TCR or CAR immune cell, wherein the TCR or CAR targets one or more tumor associated antigens selected from: BCMA, an important signaling receptor found naturally on mature B cells; often expressed by lymphoma and myeloma cells; CD19, a receptor found on the surface of almost all B cells that influences their growth, development, and activity, often expressed by leukemia, lymphoma, and myeloma cells; CD22, a receptor found primarily on the surface of mature B cells; often expressed by leukemia and lymphoma cells; CD30, a receptor that is expressed on certain types of activated immune cells, often expressed by leukemia and lymphoma cells; CD33: a surface receptor found on several types of immune cells; often expressed by leukemia cells; CD56, a protein found on both neurons and natural killer immune cells; CD123 (also known as IL-3R), a receptor found on immune cells that is involved in proliferation and differentiation, and often expressed by leukemia and lymphoma cells; CEA, a protein involved in cellular adhesion normally produced only before birth, often abnormally expressed in cancer and may contribute to metastasis; EBV-related antigens, foreign viral proteins expressed by Epstein-Barr Virus (EBV)-infected cancer cells; EGFR, a pathway that controls cell growth and is often mutated in cancer; GD2, a pathway that controls cell growth, adhesion, and migration, and is often abnormally overexpressed in cancer cells; GPC3, a cell surface protein thought to be involved in regulating growth and cell division; HER2, a pathway that controls cell growth and is commonly overexpressed in some cancers, particularly breast cancer, and is associated with metastasis; HPV-related antigens, foreign viral proteins expressed by cancer cells that develop as a consequence of having been infected with Human Papilloma Virus (HPV); MAGE antigens, the genes that produce these proteins are normally turned off in adult cells, but can become reactivated in cancer cells, flagging them as abnormal to the immune system; Mesothelin, a protein that is commonly overexpressed in cancer and may aid metastasis; MUC-1, a sugar-coated protein that is commonly overexpressed in cancer; NY-ESO-1, a protein that is normally produced only before birth, but is often abnormally expressed in cancer; PSCA, a surface protein that is found on several cell types and is often overexpressed by cancer cells; PSMA, a surface protein found on prostate cells that is often overexpressed by prostate cancer cells; ROR1, a tyrosine kinase-like orphan receptor that is mostly expressed before birth rather than in adult tissues, but is often abnormally expressed in cancer and may promote cancer cell metastasis as well as prevent cancer cell death; WT1, a protein that promotes cancer progression, is abnormally expressed in patients with cancer, especially leukemia; and Claudin 18.2: a surface protein overexpressed in some esophageal cancers and involved in invasion and survival. In some embodiments, the engineered CAR therapy is Axicabtagene ciloleucel (Yescarta®): a CD19-targeting CAR T cell immunotherapy; approved for subsets of patients with lymphoma. In some embodiments, the engineered CAR therapy is Tisagenlecleucel (Kymriah®): a CD19-targeting CAR T cell immunotherapy; approved for subsets of patients with leukemia and lymphoma. In some embodiments, the engineered CAR therapy is Lisocabtagene maraleucel (Bristol-Myers Squibb Co.): a CD19-targeting CAR T cell immunotherapy which is used to treat relapsed/refractory large B-cell lymphoma, including diffuse large B-cell lymphoma (DLBCL). In some embodiments, the engineered CAR Therapy is a BCMA CAR-T therapy, for example, but not limited to JNJ-4528 (Johnson & Johnson) and KITE-585 (Gilead). In some embodiments, the engineered CAR-T therapy is a dual specific CAR-T targeting BCMA and CD38. In some embodiments, the engineered CAR therapy is a CD20/CD22 dual targeted CAR-T cell therapy. Compositions and methods for deriving CAR immune cells are described, for example, in U.S. Pat. No. 5,359,046 (Cell Genesys); U.S. Pat. No. 5,712,149 (Cell Genesys); U.S. Pat. No. 6,103,521 (Cell Genesys); U.S. Pat. No. 7,446,190 (Memorial Sloan Kettering Cancer Center); U.S. Pat. No. 7,446,179 (City of Hope); U.S. Pat. No. 7,638,325 (U. Penn); U.S. Pat. No. 8,911,993 (U. Penn); U.S. Pat. No. 8,399,645 (St. Jude's Children's Hospital); U.S. Pat. No. 8,906,682 (U. Penn); U.S. Pat. No. 8,916,381 (U. Penn); U.S. Pat. No. 8,975,071 (U. Penn); U.S. Pat. No. 9,102,760 (U. Penn); U.S. 9,4644 (U. Penn); U.S. Pat. No. 9,855,298 (Gilead); U.S. Pat. No. 10,144,770 (St. Jude Children's Hospital); U.S. Pat. No. 10,266,580 (U. Penn); U.S. patent No. 10,189,903 (Seattle Children's Hospital); WO 2014/011988 (U. Penn); WO 2014/145252; WO 2014/153270 (Novartis AG); US 2018/0360880 (Memorial Sloan Kettering Cancer Center); WO 2017/0243 (Dana Farber Cancer Institute); WO 2016/115177 (Juno Therapeutics, Inc.); each of which is incorporated herein by reference.

In some embodiments, the immunotherapeutic is a non-engineered adoptive cell therapy. Adoptive cell therapy is an approach used to bolster the ability of the immune system to fight diseases, such as tumor and viral infections. According to this approach, immune cells, for example T cells or NK cells, are collected from a patient or donor, stimulated in the presence of antigen presenting cells bearing tumor or viral-associated antigens, and then expanded ex vivo. In some embodiments, the adoptive cell therapy is Tumor-Infiltrating Lymphocyte (TIL) Therapy, which harvests naturally occurring T cells that have already infiltrated patients' tumors, and are then activated and expanded, Then, and re-infused into patients. In some embodiments, the non-engineered adoptive cell therapy includes autologous or allogeneic immune cells, for example αβ T-cells activated to target multiple potential antigens. One strategy used to develop targeted non-engineered T-cells involves the ex vivo expansion of T-cells by antigen-specific stimulation of patient-derived (autologous) or donor-derived (allogeneic) T cells ex vivo. These strategies generally involve the isolation of peripheral blood mononuclear cells (PBMCs) and exposure of the cells to one or more tumor associated antigens. In particular, approaches to generate multi-antigen specific T-cells have focused on priming and activating T-cells with multiple targeted antigen overlapping peptide libraries, for example multiple libraries of 15mer peptides overlapping by 11 amino acids spanning the whole amino acid sequence of several target antigens (see for example commercially available overlapping peptide library products from JPT Technologies or Miltenyi). Strategies for activating ex vivo autologous or allogenic immune effector cells for targeting tumor associated antigens are described in, for example: US2011/0182870 (Baylor College of Medicine); US 2015/0010519 (Baylor College of Medicine); US2015/0017723 (Baylor College of Medicine); WO2006026746 (United States Government, Department of Health and Human Services); US 2015/0044258 (Cell Medica/Kurr Therapeutics); WO2016/154112 (Children's National Medical Center); WO 2017/203356 (Queensland Institute of Medical Research); WO 2018/005712 (Geneius Biotechnology, Inc.); Vera et al. Accelerated Production of Antigen-Specific T Cells for Pre-clinical and Clinical Applications using Gas-permeable Rapid Expansion Cultureware (G-Rex), April 2010 Journal of Immunotherapy 33(3):305-315; Shafer et al. Antigen-specific Cytotoxic T Lymphocytes can Target Chemoresistant Side-Population Tumor Cells in Hodgkin's Lymphoma; May 2010 Leukemia Lymphoma 51(5): 870-880; Quintarelli et al. High Avidity Cytotoxic T Lymphocytes Specific for a New PRAME-derived Peptide can Target Leukemic and Leukemic-precursor cells, Mar. 24, 2011 Blood 117(12): 3353-3362; Bollard et al. Manufacture of GMP-grade Cytotoxic T Lymphocytes Specific for LMP1 and LMP2 for Patients with EBV-associated Lymphoma, May 2011 Cytotherapy 13(5): 518-522; Ramos et al. Human Papillomavirus Type 16 E6/E7-Specific Cytotoxic T Lymphocytes for Adoptive Immunotherapy of HPV-associated Malignancies, January 2013 Immunotherapy 36(1): 66-76; Weber et al. Generation of tumor antigen-specific T cell lines from pediatric patients with acute lymphoblastic leukemia—implications for immunotherapy, Clinical Cancer Research 2013 Sep. 15; 19(18): 5079-5091; Ngo et al. Complementation of antigen presenting cells to generate T lymphocytes with broad target specificity, Journal of Immunotherapy. 2014 May; 37(4): 193-203; each of which is incorporated herein by reference. In some embodiments, the non-engineered, activated immune cell administered in combination or alternation with a degrader composition described herein is selected from activated CD4+ T-cells (T-helper cells), CD8+ T-cells (Cytotoxic T-Lymphocytes), CD3+/CD56+ Natural Killer T-cells (CD3+ NKT), and γδ T-cells (γδ T-cells), or combinations thereof. In some embodiments, the adoptive cell therapy is a composition comprising CD4+ T-cells (T-helper cells). In some embodiments, the adoptive cell therapy is a composition comprising CD8+ T-cells (Cytotoxic T-Lymphocytes). In some embodiments, the adoptive cell therapy is a composition comprising CD3+/CD56+ Natural Killer T-cells (CD3+ NKT). In some embodiments, the adoptive cell therapy is a composition comprising CD4+ T-cells (T-helper cells), CD8+ T-cells (Cytotoxic T-Lymphocytes), CD3+/CD56+ Natural Killer T-cells (CD3+ NKT), and γδ T6 T-cells (γδ T6 T-cells).

In some embodiments, the immunotherapy is a bi-specific T-cell engager (BiTE). A bi-specific T-cell engager directs T-cells to target and bind with a specific antigen on the surface of a cancer cell. For example, Blinatumomab (Amgen), a BiTE has recently been approved as a second line therapy in Philadelphia chromosome-negative relapsed or refractory acute lymphoblastic leukemia. Blinatumomab is given by continuous intravenous infusion in 4-week cycles.

In certain embodiments, the additional therapeutically active agent is an additional inhibitor of Ikaros (“IKZF1”) and/or Aiolos (“IKZF3”). In another embodiment, the additional therapeutically active agent is an inhibitor of Helios (“IKZF2”). In another embodiment, the additional therapeutically active agent is an inhibitor of Eos (“IKZF4”). In another embodiment, the additional therapeutically active agent is an inhibitor of Pegasus (“IKZF5”). In another embodiment, the additional therapeutically active agent is a cereblon ligand.

Non-limiting examples of cereblon ligands that may be used in combination with a compound of the present invention include: thalidomide, lenalidomide, pomalidomide, and iberdomide.

In another embodiment the additional compound that may be used in combination with a compound of the present invention is selected from those described in WO2012/175481, WO2015/085172, WO2015/085172, WO2017/067530, WO2017/121388, WO2017/201069, WO2018/108147, WO2018/118947, WO2019/038717, WO2019/191112, WO2020/006233, WO2020/006262, WO2020/006265, or WO2020/012334.

In another embodiment the additional compound that may be used in combination with a compound of the present invention is selected from those described in WO2019/060693, WO2019/060742, WO2019/133531, WO2019/140380, WO2019/140387, WO2010/010177, WO2020/010210, or WO2020/010227.

In another embodiment the additional compound that may be used in combination with a compound of the present invention is selected from those described in WO2015/160845, WO2016/118666, WO2016/149668, WO2016/197032, WO2016/197114, WO2017/011371, WO2017/0115901, WO2017/030814, WO2017/176708, WO2018/053354, WO2018/0716060, WO2018/102067, WO2018/118598, WO2018/119357, WO2018/119441, WO2018/119448, WO2018/140809, WO2018/226542, WO2019/023553, WO2019/099926, WO2019/195201, WO2019/195609, WO2019/199816, WO2020/023851, WO2020/041331, or WO2020/051564.

In another embodiment the additional compound that may be used in combination with a compound of the present invention is selected from those described in WO2016/105518, WO2017/007612, WO2017/024317, WO2017/024318, WO2017/024319, WO2017/117473, WO2017/117474, WO2017/185036, WO2018/064589, WO2018/148440, WO2018/148443, WO2018/226978, WO2019/014429, WO2019/079701, WO2019/094718, WO2019/094955, WO2019/118893, WO2019/165229, WO2020/006262, WO2020/018788, WO2020/069105, WO2020/069117, or WO2020/069125.

In another embodiment the additional compound that may be used in combination with a compound of the present invention is selected from those described in WO2017/197036, WO2017/197046, WO2017/197051, WO2017/197055, WO2017/197056, WO 2017/115218, WO2018/220149, WO2018/237026, WO2019/099868, WO2019/121562, WO2019/149922, WO2019/191112, WO2019/204354, WO2019/236483, or WO2020/051235.

In some embodiments, the bioactive agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (AVASTIN®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include RITUXAN® (rituximab); ZENAPAX® (daclizumab); SIMULECT® (basiliximab); SYNAGIS® (palivizumab); REMICADE® (infliximab); HERCEPTIN® (trastuzumab); MYLOTARG® (gemtuzumab ozogamicin); CAMPATH® (alemtuzumab); ZEVALIN® (ibritumomab tiuxetan); HUMIRA® (adalimumab); XOLAIR® (omalizumab); BEXXAR® (tositumomab−1-131); RAPTIVA® (efalizumab); ERBITUX® (cetuximab); AVASTIN® (bevacizumab); TYSABRI® (natalizumab); ACTEMRA® (tocilizumab); VECTIBIX® (panitumumab); LUCENTIS® (ranibizumab); SOURIS® (eculizumab); CIMZIA® (certolizumab pegol); SIMPONI® (golimumab); ILARIS® (canakinumab); STELARA® (ustekinumab); ARZERRA® (ofatumumab); PROLIA® (denosumab); NUMAX® (motavizumab); ABTHRAX® (raxibacumab); BENLYSTA® (belimumab); YERVOY® (ipilimumab); ADCETRIS® (brentuximab vedotin); PERJETA® (pertuzumab); KADCYLA® (ado-trastuzumab emtansine); and GAZYVA® (obinutuzumab). Also included are antibody-drug conjugates.

The combination therapy may include a therapeutic agent which is a non-drug treatment. For example, the compound could be administered in addition to radiation therapy, cryotherapy, hyperthermia, and/or surgical excision of tumor tissue.

In certain embodiments the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

In certain embodiments the second therapeutic agent is administered on a different dosage schedule than the compound of the present invention. For example the second therapeutic agent may have a treatment holiday of 1 day, 2 days, 3 days, 4 days, 5 days, 6 days, 7 days, 8 days, 9 days, 10 days, 11 days, 12 days, 13 days, or 14 days per treatment cycle. In another embodiment the first therapeutic agent has a treatment holiday. For example the first therapeutic agent may have a treatment holiday of 1 day, 2 days, 3 days, 4 days, 5 days, 6 days, 7 days, 8 days, 9 days, 10 days, 11 days, 12 days, 13 days, or 14 days per treatment cycle. In certain embodiments both the first and second therapeutic have a treatment holiday.

### V. Pharmaceutical Compositions

Any of the compounds as disclosed herein can be administered as the neat chemical, but are more typically administered as a pharmaceutical composition, that includes an effective amount for a host, typically a human, in need of such treatment for any of the disorders described herein. Accordingly, the disclosure provides pharmaceutical compositions comprising an effective amount of compound or pharmaceutically acceptable salt together with at least one pharmaceutically acceptable carrier for any of the uses described herein. The pharmaceutical composition may contain a compound or salt as the only active agent, or, in an alternative embodiment, the compound and at least one additional active agent.

In certain embodiments the pharmaceutical composition is in a dosage form that contains from about 0.0005 mg to about 2000 mg, from about 0.001 mg to about 1000 mg, from about 0.001 mg to about 600 mg, or from about 0.001 mg to about 1, 5, 10, 15, 20, 25, 50, 100, 200 or 300 mg mg of the active compound. In another embodiment the pharmaceutical composition is in a dosage form that contains from about 0.01 mg to about 1, 5, 10, 15, 20, 25, 50 or 100 mg, from about 0.05 mg to about 1, 5, 10, 15, 20, 25, 50 or 100 mg, from about 0.1 mg to about 1, 5, 10, 15, 20, 25 or 50 mg, from about 0.02 mg to about 1, 5, 10, 15, 20, 25 or 50 mg of the active compound, from about 0.5 mg to about 1, 5, 10, 15, 20, 25 or 50 mg. In another embodiment the pharmaceutical composition is in a dosage form that contains from about 0.01 mg to about 10 mg, from about 0.05 mg to about 8 mg, or from about 0.05 mg to about 6 mg, or from about 0.05 mg to about 5 mg of the active compound. In another embodiment the pharmaceutical composition is in a dosage form that contains from about 0.1 mg to about 10 mg, from about 0.5 mg to about 8 mg, or from about 0.5 mg to about 6 mg, or from about 0.5 mg to about 5 mg of the active compound. Nonlimiting examples are dosage forms with at least about 0.0005, 0.001, 0.01, 0.1, 1, 2.5, 5, 10, 25, 50, 100, 200, 250, 300, 400, 500, 600, 700, or 750 mg of active compound, or its salt. Alternative nonlimiting examples are dosage forms with not greater than about 0.01, 0.1, 1, 2.5, 5, 10, 25, 50, 100, 200, 250, 300, 400, 500, 600, 700, or 750 mg of active compound, or its salt.

In some embodiments, compounds disclosed herein or used as described are administered once a day (QD), twice a day (BID), or three times a day (TID). In some embodiments, compounds disclosed herein or used as described are administered at least once a day for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 35 days, at least 45 days, at least 60 days, at least 75 days, at least 90 days, at least 120 days, at least 150 days, at least 180 days, or longer.

In certain embodiments the compound of the present invention is administered once a day, twice a day, three times a day, or four times a day.

In certain embodiments the compound of the present invention is administered orally once a day. In certain embodiments the compound of the present invention is administered orally twice a day. In certain embodiments the compound of the present invention is administered orally three times a day. In certain embodiments the compound of the present invention is administered orally four times a day.

In certain embodiments the compound of the present invention is administered intravenously once a day. In certain embodiments the compound of the present invention is administered intravenously twice a day. In certain embodiments the compound of the present invention is administered intravenously three times a day. In certain embodiments the compound of the present invention is administered intravenously four times a day.

In some embodiments the compound of the present invention is administered with a treatment holiday in between treatment cycles. For example the compound may have a treatment holiday of 1 day, 2 days, 3 days, 4 days, 5 days, 6 days, 7 days, 8 days, 9 days, 10 days, 11 days, 12 days, 13 days, or 14 days per treatment cycle.

In some embodiments a loading dose is administered to begin treatment. For example, the compound may be administered in a dosage that is at least about 1.5×, 2×, 2.5×, 3×, 3.5×, 4×, 4.5×, 5×, 5.5×, 6×, 6.5×, 7×, 7.5×, 8×, 8.5×, 9×, 9.5×, or 10× higher dose to initiate treatment than the maintenance dose treatment cycle. Additional exemplary loading doses include at least about 1.5×, 2×, 2.5×, 3×, 3.5×, 4×, 4.5×, 5×, 5.5×, 5×, 6.5×, 7×, 7.5×, 8×, 8.5×, 9×, 9.5×, or 10× higher dose on the first 1, 2, 3, 4, 5, 6, 7, 8, 9, or 10 days of treatment followed by the maintenance dose on the remaining days of treatment in the treatment cycle.

The pharmaceutical composition may also include a molar ratio of the active compound and an additional therapeutically active agent. In non-limiting illustrative embodiments the pharmaceutical composition may contain a molar ratio of about up to 0.5:1, about up to 1:1, about up to 2:1, about up to 3:1 or from about up to 1.5:1 to about up to 4:1 of an anti-inflammatory or immunosuppressing agent to the compound of the present invention.

In another embodiment, the tricyclic compound is administered in an effective amount to a host, typically a human in need thereof with a loading dose followed by a maintenance dose. In certain embodiments, the loading dose is at least about 1.5, 2 or 3 times the maintenance dose. In certain embodiments, the loading dose is provided for 1, 2, 3, 4, 5, 6, or 7 days before initiation of the maintenance dose.

Compounds disclosed herein may be administered orally, topically, parenterally, by inhalation or spray, sublingually, via implant, including ocular implant, transdermally, via buccal administration, rectally, as an ophthalmic solution, injection, including ocular injection, intravenous, intra-aortal, intracranial, subdermal, intraperitoneal, subcutaneous, transnasal, sublingual, or rectal or by other means, in dosage unit formulations containing conventional pharmaceutically acceptable carriers. For ocular delivery, the compound can be administered, as desired, for example, via intravitreal, intrastromal, intracameral, sub-tenon, sub-retinal, retro-bulbar, peribulbar, suprachoroidal, conjunctival, subconjunctival, episcleral, periocular, transscleral, retrobulbar, posterior juxtascleral, circumcorneal, or tear duct injections, or through a mucus, mucin, or a mucosal barrier, in an immediate or controlled release fashion or via an ocular device.

The pharmaceutical composition may be formulated as any pharmaceutically useful form, e.g., as an aerosol, a cream, a gel, a pill, an injection or infusion solution, a capsule, a tablet, a syrup, a transdermal patch, a subcutaneous patch, a dry powder, an inhalation formulation, in a medical device, suppository, buccal, or sublingual formulation, parenteral formulation, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Pharmaceutically acceptable carriers are carriers that do not cause any severe adverse reactions in the human body when dosed in the amount that would be used in the corresponding pharmaceutical composition. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present invention.

The pharmaceutical compositions/combinations can be formulated for oral administration. These compositions can contain any amount of active compound that achieves the desired result, for example between 0.1 and 99 weight % (wt. %) of the compound including for example at least about 5 wt. % of the compound. Some embodiments contain from about 25 wt. % to about 50 wt. % or from about 5 wt. % to about 75 wt. % of the compound.

A pharmaceutically or therapeutically effective amount of the composition will be delivered to the patient. The precise effective amount will vary from patient to patient, and will depend upon the species, age, the subject's size and health, the nature and extent of the condition being treated, recommendations of the treating physician, and the therapeutics or combination of therapeutics selected for administration. The effective amount for a given situation can be determined by routine experimentation. For purposes of the disclosure, a therapeutic amount may for example be in the range of about 0.01 mg/kg to about 250 mg/kg body weight, more typically about 0.1 mg/kg to about 10 mg/kg, in at least one dose. The subject can be administered as many doses as is required to reduce and/or alleviate the signs, symptoms, or causes of the disorder in question, or bring about any other desired alteration of a biological system. When desired, formulations can be prepared with enteric coatings adapted for sustained or controlled release administration of the active ingredient.

In certain embodiments the dose ranges from about 0.01-100 mg/kg of patient bodyweight, for example about 0.01 mg/kg, about 0.05 mg/kg, about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg.

In certain embodiments a therapeutic amount may for example be in the range of about 0.0001 mg/kg to about 25 mg/kg body weight. The subject can be administered as many doses as is required to reduce and/or alleviate the signs, symptoms, or causes of the disorder in question, or bring about any other desired alteration of a biological system. When desired, formulations can be prepared with enteric coatings adapted for sustained or controlled release administration of the active ingredient.

In certain embodiments the dose ranges from about 0.001-10 mg/kg of patient bodyweight, for example about 0.0001 mg/kg, about 0.0005 mg/kg, about 0.001 mg/kg, about 0.005 mg/kg, about 0.01 mg/kg, about 0.05 mg/kg, about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, about 1 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6.0 mg/kg, about 6.5 mg/kg, about 7.0 mg/kg, about 7.5 mg/kg, about 8.0 mg/kg, about 8.5 mg/kg, about 9.0 mg/kg, about 9.5 mg/kg, or about 10 mg/kg.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packed tablets, capsules, and powders in vials or ampoules. Also, the unit dosage form can be a capsule, tablet, cachet, or lozenge itself, or it can be the appropriate number of any of these in packaged form.

In certain embodiments the compound is administered as a pharmaceutically acceptable salt. Non-limiting examples of pharmaceutically acceptable salts include: acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

Thus, the composition of the disclosure can be administered as a pharmaceutical formulation including one suitable for oral (including buccal and sub-lingual), rectal, nasal, topical, transdermal, pulmonary, vaginal or parenteral (including intramuscular, intra-arterial, intrathecal, subcutaneous and intravenous), injections, inhalation or spray, intra-aortal, intracranial, subdermal, intraperitioneal, subcutaneous, or by other means of administration containing conventional pharmaceutically acceptable carriers. A typical manner of administration is oral, topical or intravenous, using a convenient daily dosage regimen which can be adjusted according to the degree of affliction.

Depending on the intended mode of administration, the pharmaceutical compositions can be in the form of solid, semi-solid or liquid dosage forms, such as, for example, tablets, suppositories, pills, capsules, powders, liquids, syrup, suspensions, creams, ointments, lotions, paste, gel, spray, aerosol, foam, or oil, injection or infusion solution, a transdermal patch, a subcutaneous patch, an inhalation formulation, in a medical device, suppository, buccal, or sublingual formulation, parenteral formulation, or an ophthalmic solution, or the like, preferably in unit dosage form suitable for single administration of a precise dosage.

Some dosage forms, such as tablets and capsules, are subdivided into suitably sized unit doses containing appropriate quantities of the active components, e.g., an effective amount to achieve the desired purpose. The compositions will include an effective amount of the selected drug in combination with a pharmaceutically acceptable carrier and, in addition, can include other pharmaceutical agents, adjuvants, diluents, buffers, and the like.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to adjuvants, binders, buffering agents, coloring agents, diluents, disintegrants, excipients, emulsifiers, flavorants, gels, glidents, lubricants, preservatives, stabilizers, surfactants, solubilizer, tableting agents, wetting agents or solidifying material.

Some carriers may be listed in more than one class, for example vegetable oil may be used as a lubricant in some formulations and a diluent in others.

Exemplary pharmaceutically acceptable carriers include sugars, starches, celluloses, powdered tragacanth, malt, gelatin; talc, petroleum jelly, lanoline, polyethylene glycols, alcohols, transdermal enhancers and vegetable oils. Optional active agents may be included in a pharmaceutical composition, which do not substantially interfere with the activity of the compound of the present invention.

Some excipients include, but are not limited, to liquids such as water, saline, glycerol, polyethylene glycol, hyaluronic acid, ethanol, and the like. The compound can be provided, for example, in the form of a solid, a liquid, spray dried material, a microparticle, nanoparticle, controlled release system, etc., as desired according to the goal of the therapy. Suitable excipients for non-liquid formulations are also known to those of skill in the art. A thorough discussion of pharmaceutically acceptable excipients and salts is available in Remington's Pharmaceutical Sciences, 18th Edition (Easton, Pennsylvania: Mack Publishing Company, 1990).

Additionally, auxiliary substances, such as wetting or emulsifying agents, biological buffering substances, surfactants, and the like, can be present in such vehicles. A biological buffer can be any solution which is pharmacologically acceptable, and which provides the formulation with the desired pH, i.e., a pH in the physiologically acceptable range. Examples of buffer solutions include saline, phosphate buffered saline, Tris buffered saline, Hank's buffered saline, and the like.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington's Pharmaceutical Sciences, referenced above.

In yet another embodiment provided is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and, thiolated polymers (carboxymethyl cellulose-cysteine, polycarbophil-cysteine, chitosan-thiobutylamidine, chitosan-thioglycolic acid, chitosan-glutathione conjugates).

In certain embodiments the excipient is selected from butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

The pharmaceutical compositions/combinations can be formulated for oral administration. For oral administration, the composition will generally take the form of a tablet, capsule, a softgel capsule or can be an aqueous or nonaqueous solution, suspension or syrup. Tablets and capsules are typical oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

For ocular delivery, the compound can be administered, as desired, for example, via intravitreal, intrastromal, intracameral, sub-tenon, sub-retinal, retro-bulbar, peribulbar, suprachorodial, conjunctival, subconjunctival, episcleral, periocular, transscleral, retrobulbar, posterior juxtascleral, circumcorneal, or tear duct injections, or through a mucus, mucin, or a mucosal barrier, in an immediate or controlled release fashion or via an ocular device.

Parenteral formulations can be prepared in conventional forms, either as liquid solutions or suspensions, solid forms suitable for solubilization or suspension in liquid prior to injection, or as emulsions. Typically, sterile injectable suspensions are formulated according to techniques known in the art using suitable carriers, dispersing or wetting agents and suspending agents. The sterile injectable formulation can also be a sterile injectable solution or a suspension in a acceptably nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as a continuous infusion system. A formulation provided by the disclosure can be administered using a syringe, injector, pump, or any other device recognized in the art for parenteral administration.

Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Sterile injectable solutions are prepared by incorporating one or more of the compounds of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, typical methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

Alternatively, the pharmaceutical compositions of the disclosure can be administered in the form of suppositories for rectal administration. These can be prepared by mixing the agent with a suitable nonirritating excipient which is solid at room temperature but liquid at the rectal temperature and therefore will melt in the rectum to release the drug. Such materials include cocoa butter, beeswax and polyethylene glycols.

The pharmaceutical compositions of the disclosure can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, propellants such as fluorocarbons or nitrogen, and/or other conventional solubilizing or dispersing agents.

Formulations for buccal administration include tablets, lozenges, gels and the like. Alternatively, buccal administration can be effected using a transmucosal delivery system as known to those skilled in the art. The compounds of the disclosure can also be delivered through the skin or muscosal tissue using conventional transdermal drug delivery systems, i.e., transdermal “patches” wherein the agent is typically contained within a laminated structure that serves as a drug delivery device to be affixed to the body surface. In such a structure, the drug composition is typically contained in a layer, or “reservoir,” underlying an upper backing layer. The laminated device can contain a single reservoir, or it can contain multiple reservoirs. In certain embodiments, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like.

Alternatively, the drug-containing reservoir and skin contact adhesive are present as separate and distinct layers, with the adhesive underlying the reservoir which, in this case, can be either a polymeric matrix as described above, or it can be a liquid or gel reservoir, or can take some other form. The backing layer in these laminates, which serves as the upper surface of the device, functions as the primary structural element of the laminated structure and provides the device with much of its flexibility. The material selected for the backing layer should be substantially impermeable to the active agent and any other materials that are present.

The compositions of the disclosure can be formulated for aerosol administration, particularly to the respiratory tract and including intranasal administration. The compound may, for example generally have a small particle size for example of the order of 5 microns or less. Such a particle size can be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol can conveniently also contain a surfactant such as lecithin. The dose of drug can be controlled by a metered valve.

Alternatively, the active ingredients can be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition can be presented in unit dose form for example in capsules or cartridges of e.g., gelatin or blister packs from which the powder can be administered by means of an inhaler.

Formulations suitable for rectal administration are typically presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. In certain embodiments, microneedle patches or devices are provided for delivery of drugs across or into biological tissue, particularly the skin. The microneedle patches or devices permit drug delivery at clinically relevant rates across or into skin or other tissue barriers, with minimal or no damage, pain, or irritation to the tissue.

Formulations suitable for administration to the lungs can be delivered by a wide range of passive breath driven and active power driven single/-multiple dose dry powder inhalers (DPI). The devices most commonly used for respiratory delivery include nebulizers, metered-dose inhalers, and dry powder inhalers. Several types of nebulizers are available, including jet nebulizers, ultrasonic nebulizers, and vibrating mesh nebulizers. Selection of a suitable lung delivery device depends on parameters, such as nature of the drug and its formulation, the site of action, and pathophysiology of the lung.

### VI. General Synthesis

The compounds described herein can be prepared by methods known by those skilled in the art. In one non-limiting example, the disclosed compounds can be made using the schemes below.

Compounds of the present invention with stereocenters may be drawn without stereochemistry for convenience. One skilled in the art will recognize that pure or enriched enantiomers and diastereomers can be prepared by methods known in the art. Examples of methods to obtain optically active materials include at least the following:
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the enantiomer is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step in the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e. chirality) in the product, which may be achieved by chiral catalysts or chiral auxiliaries;
vi) diastereomer separations—a technique whereby a racemic compound is reaction with an enantiomerically pure reagent (the chiral auxiliary) that converts the individual enantiomers to diastereomers. The resulting diastereomers are then separated by chromatography or crystallization by virtue of their now more distinct structural differences the chiral auxiliary later removed to obtain the desired enantiomer;
vii) first- and second-order asymmetric transformations—a technique whereby diastereomers from the racemate quickly equilibrate to yield a preponderance in solution of the diastereomer from the desired enantiomer of where preferential crystallization of the diastereomer from the desired enantiomer perturbs the equilibrium such that eventually in principle all the material is converted to the crystalline diastereomer from the desired enantiomers. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase (including vial chiral HPLC). The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent;
xiii) transport across chiral membranes—a technique whereby a racemate is place in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through;
xiv) simulated moving bed chromatography is used in certain embodiments. A wide variety of chiral stationary phases are commercially available.

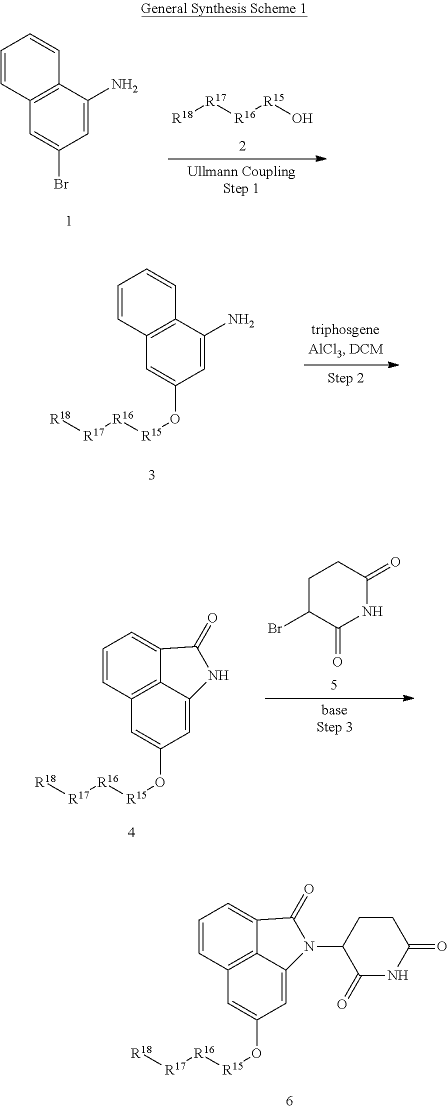

A compound of Formula XV can be synthesized according to the route provided in General Synthesis Scheme 1. In step 1, compound 1 is reacted with 2 in the presence of a copper catalyst (for example, copper(I) iodide, copper(I) chloride, or alternatively another suitable copper catalyst used in Ullmann coupling conditions), a ligand (for example, bipyridine, 1,10-phenanthroline, dimethylethylenediamine, or alternatively another suitable ligand used in Ullmann coupling conditions), and a base (for example, cesium carbonate, potassium carbonate, tribasic potassium phosphate, or alternatively another suitable base used in Ullmann coupling conditions) in organic solvent (for example, dimethylsulfoxide, acetonitrile, or dioxane) at elevated temperature to afford 3. In step 2, compound 3 is reacted with triphosgene in the presence of aluminum trichloride in dichloromethane to afford 4. In step 3, compound 4 is reacted with a base (for example, sodium hydride) in an organic solvent (for example, tetrahydrofuran or dichloromethane) followed by the addition of 5 to afford 6.

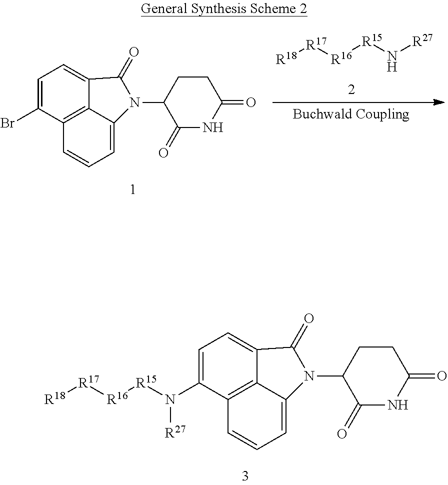

A compound of Formula XVI can be synthesized according to the route provided in General Synthesis Scheme 2. Compound 1 is reacted with 2 in the presence of a palladium catalyst (for example, palladium(II) acetate, Pd₂(dba)₃, or alternatively another suitable palladium catalyst used in Buchwald-Hartwig coupling conditions), a phosphine ligand (for example, BINAP, XantPhos, or alternatively another suitable phosphine ligand used in Buchwald-Hartwig coupling conditions), and a base (for example, potassium tert-butoxide, cesium carbonate, or alternatively another suitable base used in Buchwald-Hartwig coupling conditions) in organic solvent (for example, toluene, THF, dioxane, or DMF) at elevated temperature to afford 3.

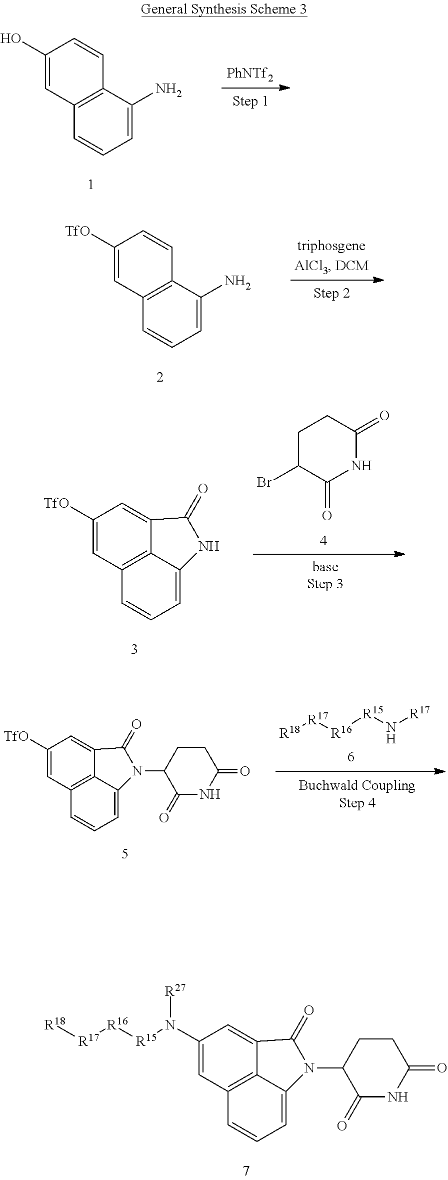

A compound of Formula XVI can be synthesized according to the route provided in General Synthesis Scheme 3. In step 1, compound 1 is reacted with phenyl triflimide in the presence of a base (for example, pyridine, triethylamine, or alternatively another suitable base used in triflating conditions) in organic solvent (for example, dichloromethane or toluene) to afford 2. In step 2, compound 2 is reacted with triphosgene in the presence of aluminum trichloride in dichloromethane to afford 3. In step 3, compound 3 is reacted with a base (for example, sodium hydride) in an organic solvent (for example, tetrahydrofuran or dichloromethane) followed by the addition of 4 to afford 5. In step 4, compound 5 is reacted with 6 in the presence of a palladium catalyst (for example, palladium(II) acetate, Pd₂(dba)₃, or alternatively another suitable palladium catalyst used in Buchwald-Hartwig coupling conditions), a phosphine ligand (for example, BINAP, XantPhos, or alternatively another suitable phosphine ligand used in Buchwald-Hartwig coupling conditions), and a base (for example, potassium tert-butoxide, cesium carbonate, or alternatively another suitable base used in Buchwald-Hartwig coupling conditions) in organic solvent (for example, toluene, THF, dioxane, or DMF) at elevated temperature to afford 7.

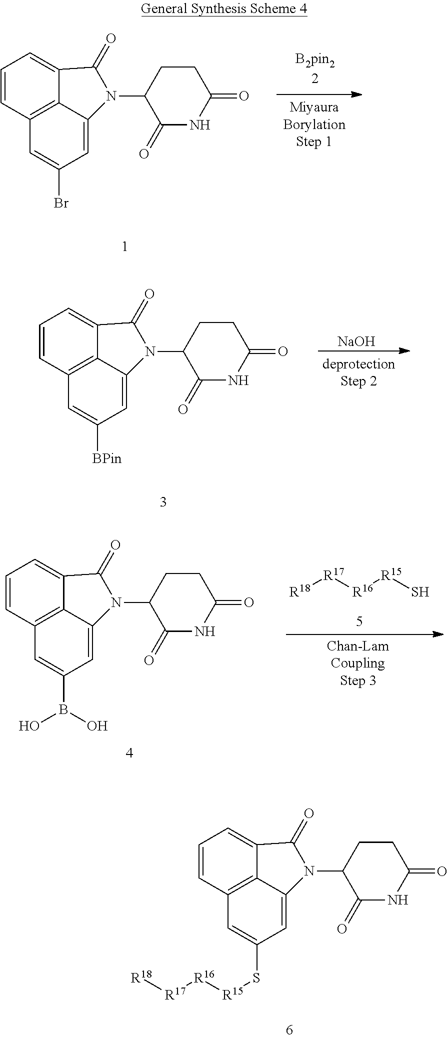

A compound of Formula XV can be synthesized according to the route provided in General Synthesis Scheme 4. In step 1, compound 1 is reacted with 2 in the presence of a palladium catalyst (for example, PdCl₂(dppf), PdCl₂(PPh₃), or alternatively another suitable palladium catalyst used in Miyaura coupling conditions), a ligand (for example, XPhos, PPh₃, or alternatively another suitable ligand used in Miyaura coupling conditions), and a base (for example, potassium acetate, potassium ethoxide, potassium carbonate, or alternatively another suitable base used in Miyaura coupling conditions) in organic solvent (for example, toluene, DMA, or dioxane) at elevated temperature to afford 3. In step 2, compound 3 is reacted with NaOH under aqueous conditions at elevated temperature to afford 4. In step 3, compound 4 is reacted with 5 in the presence of a copper catalyst (for example, copper(II) bromide, copper(II) acetate, or alternatively another suitable copper catalyst used in Chan-Lam coupling conditions) and a base (for example, pyridine, 4-dimethylaminopyridine, potassium tert-butoxide, or alternatively another suitable base used in Chan-Lam coupling conditions) in organic solvent (for example, methanol, acetonitrile, or dichloromethane) under ambient air to afford 6.

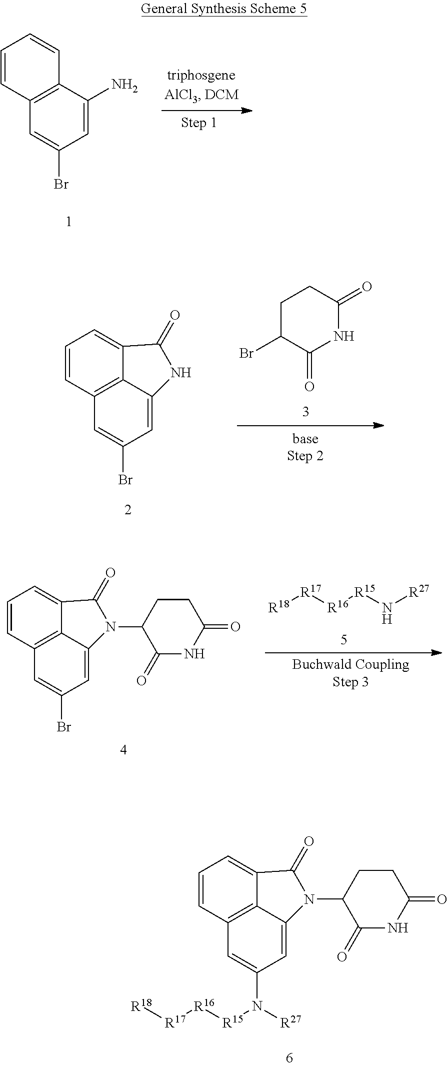

A compound of Formula XV can be synthesized according to the route provided in General Synthesis Scheme 5. In step 1, compound 1 is reacted with triphosgene in the presence of aluminum trichloride in dichloromethane to afford 2. In step 2, compound 2 is reacted with a base (for example, sodium hydride) in an organic solvent (for example, tetrahydrofuran or dichloromethane) followed by the addition of 3 to afford 4. In step 3, compound 4 is reacted with 5 in the presence of a palladium catalyst (for example, palladium(II) acetate, Pd₂(dba)₃, or alternatively another suitable palladium catalyst used in Buchwald-Hartwig coupling conditions), a phosphine ligand (for example, BINAP, XantPhos, or alternatively another suitable phosphine ligand used in Buchwald-Hartwig coupling conditions), and a base (for example, potassium tert-butoxide, cesium carbonate, or alternatively another suitable base used in Buchwald-Hartwig coupling conditions) in organic solvent (for example, toluene, THF, dioxane, or DMF) at elevated temperature to afford 6.

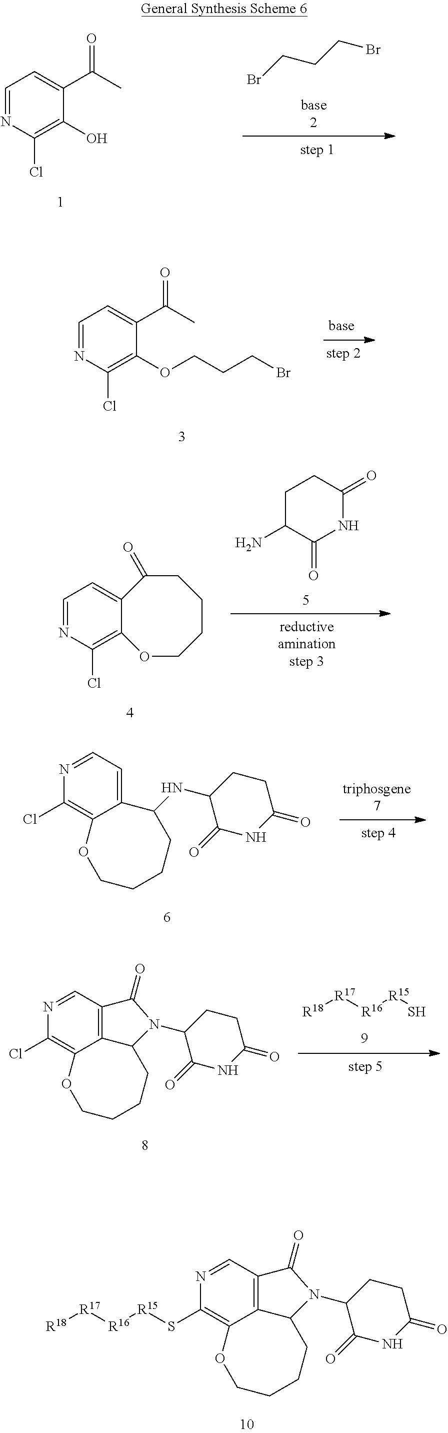

A compound of Formula XV can be synthesized according to the route provided in General Synthesis Scheme 6. In step 1, intermediate 1 is reacted with 2 in the presence of base (for example potassium carbonate, cesium carbonate, or other suitable base used in phenol alkylation conditions) in organic solvent (for example DMF, DMA, or acetonitrile) at elevated temperature to provide 3. In step 2, 3 is reacted base (for example, LDA, LiHMDS, or other suitable strong, sterically hindered base). In step 3, 4 is reacted with 5 in the presence of a mild reductant (for example, sodium triacetoxyborohydride, sodium cyanoborohydride, or other suitable hydride reductant used in reductive amination conditions) in organic solvent (for example methanol, acetonitrile, or dichloromethane) to provide 6. In step 4, 6 is reacted with triphosgene in the presence of aluminum trichloride in dichloromethane to afford 8. In step 5, 8 is reacted with 9 in organic solvent (for example DMF, DMA, or dioxane) at elevated temperature to afford 10.

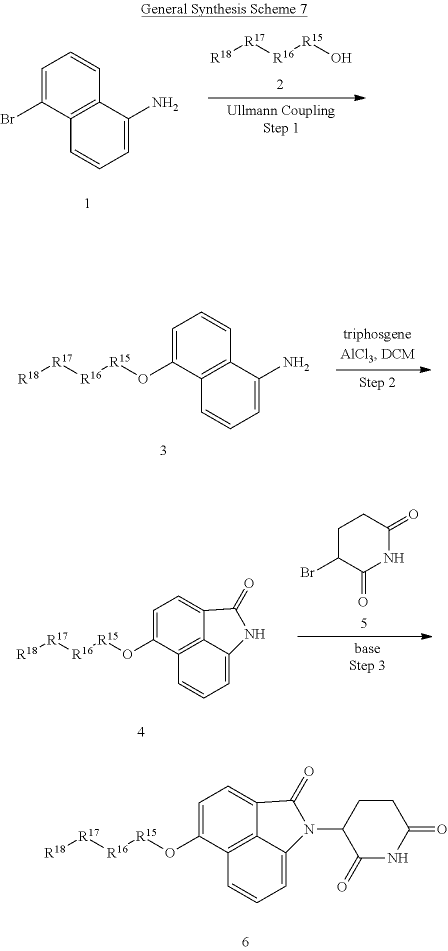

A compound of Formula XVI can be synthesized according to the route provided in General Synthesis Scheme 7. In step 1, compound 1 is reacted with 2 in the presence of a copper catalyst (for example, copper(I) iodide, copper(I) chloride, or alternatively another suitable copper catalyst used in Ullmann coupling conditions), a ligand (for example, bipyridine, 1,10-phenanthroline, dimethylethylenediamine, or alternatively another suitable ligand used in Ullmann coupling conditions), and a base (for example, cesium carbonate, potassium carbonate, tribasic potassium phosphate, or alternatively another suitable base used in Ullmann coupling conditions) in organic solvent (for example, dimethylsulfoxide, acetonitrile, or dioxane) at elevated temperature to afford 3. In step 2, compound 3 is reacted with triphosgene in the presence of aluminum trichloride in dichloromethane to afford 4. In step 3, compound 4 is reacted with a base (for example, sodium hydride) in an organic solvent (for example, tetrahydrofuran or dichloromethane) followed by the addition of 5 to afford 6.

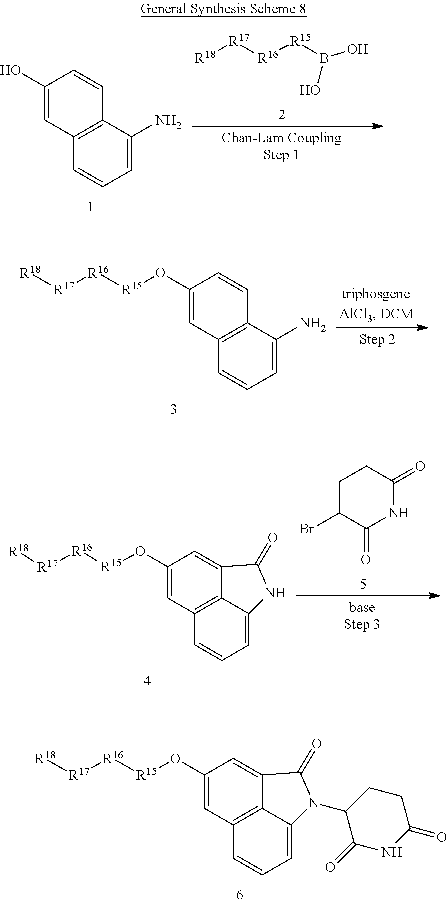

A compound of Formula XVI can be synthesized according to the route provided in General Synthesis Scheme 8. In step 1, compound 1 is reacted with 2 in the presence of a copper catalyst (for example, copper(II) bromide, copper(II) acetate, or alternatively another suitable copper catalyst used in Chan-Lam coupling conditions) and a base (for example, pyridine, 4-dimethylaminopyridine, potassium tert-butoxide, or alternatively another suitable base used in Chan-Lam coupling conditions) in organic solvent (for example, methanol, acetonitrile, or dichloromethane) under ambient air to afford 3. In step 2, compound 3 is reacted with triphosgene in the presence of aluminum trichloride in dichloromethane to afford 4. In step 3, compound 4 is reacted with a base (for example, sodium hydride) in an organic solvent (for example, tetrahydrofuran or dichloromethane) followed by the addition of 5 to afford 6.

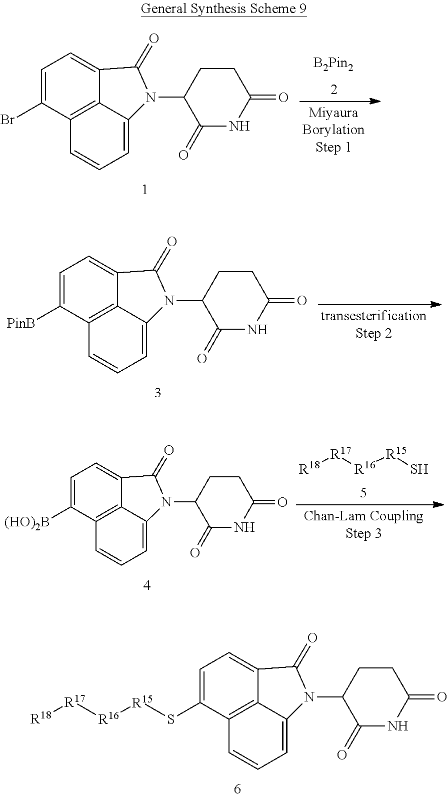

A compound of Formula XVI can be synthesized according to the route provided in General Synthesis Scheme 9. In step 1, compound 1 is reacted with 2 in the presence of a palladium catalyst (for example, PdCl₂(dppf), PdCl₂(PPh₃), or alternatively another suitable palladium catalyst used in Miyaura coupling conditions), a ligand (for example, XPhos, PPh₃, or alternatively another suitable ligand used in Miyaura coupling conditions), and a base (for example, potassium acetate, potassium ethoxide, potassium carbonate, or alternatively another suitable base used in Miyaura coupling conditions) in organic solvent (for example, toluene, DMA, or dioxane) at elevated temperature to afford 3. In step 2, compound 3 undergoes transesterification to afford 4. In step 3, compound 4 is reacted with 5 in the presence of a copper catalyst (for example, copper(II) bromide, copper(II) acetate, or alternatively another suitable copper catalyst used in Chan-Lam coupling conditions) and a base (for example, pyridine, 4-dimethylaminopyridine, potassium tert-butoxide, or alternatively another suitable base used in Chan-Lam coupling conditions) in organic solvent (for example, methanol, acetonitrile, or dichloromethane) under ambient air to afford 6.

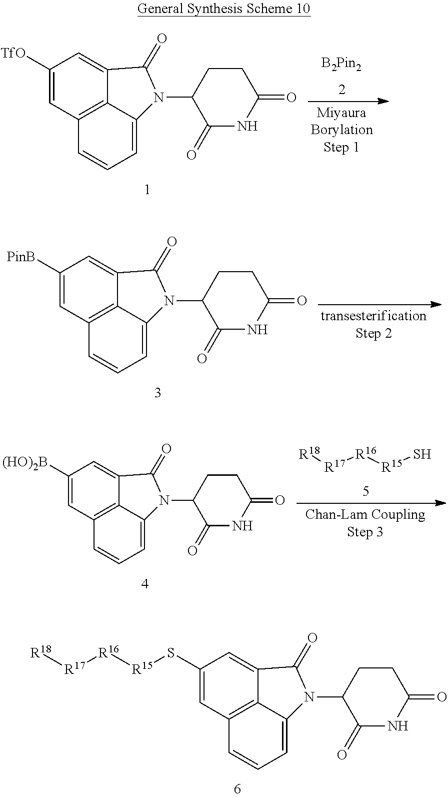

A compound of Formula XVI can be synthesized according to the route provided in General Synthesis Scheme 10. In step 1, compound 1 is reacted with 2 in the presence of a palladium catalyst (for example, PdCl₂(dppf), PdCl₂(PPh₃), or alternatively another suitable palladium catalyst used in Miyaura coupling conditions), a ligand (for example, XPhos, PPh₃, or alternatively another suitable ligand used in Miyaura coupling conditions), and a base (for example, potassium acetate, potassium ethoxide, potassium carbonate, or alternatively another suitable base used in Miyaura coupling conditions) in organic solvent (for example, toluene, DMA, or dioxane) at elevated temperature to afford 3. In step 2, compound 3 undergoes transesterification to afford 4. In step 3, compound 4 is reacted with 5 in the presence of a copper catalyst (for example, copper(II) bromide, copper(II) acetate, or alternatively another suitable copper catalyst used in Chan-Lam coupling conditions) and a base (for example, pyridine, 4-dimethylaminopyridine, potassium tert-butoxide, or alternatively another suitable base used in Chan-Lam coupling conditions) in organic solvent (for example, methanol, acetonitrile, or dichloromethane) under ambient air to afford 6.

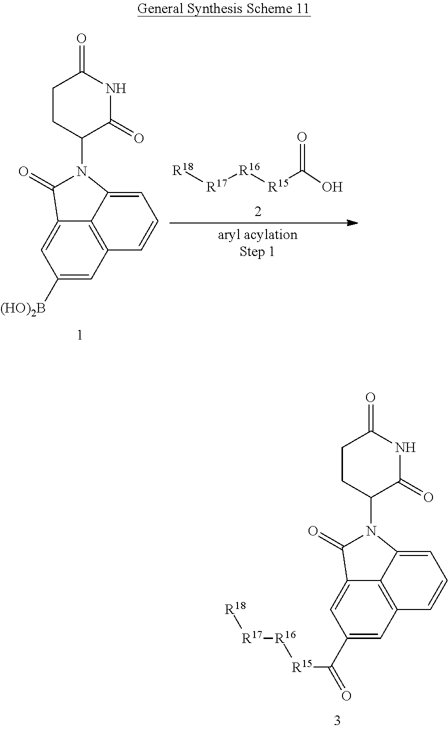

A compound of Formula XVI can be synthesized according to the route provided in General Synthesis Scheme 11. In step 1, compound 1 is reacted with 2 in the presence of a palladium catalyst (for example, Pd(OAc)₂, Pd(PPh₃)₄, or alternatively another suitable palladium catalyst), a ligand (for example, P(p-MeOPh)₃, PPh₃, PCy₃ or alternatively another suitable ligand), water, and pivalic anhydride in organic solvent (for example, dimethoxyethane, THF, or toluene) at elevated temperature to afford 3.

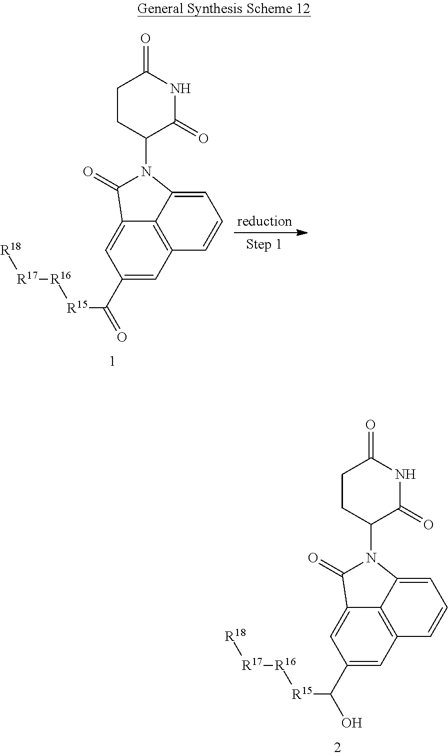

A compound of Formula XVI can be synthesized according to the route provided in General Synthesis Scheme 12. In step 1, compound 1 is reacted with a suitable carbonyl reductant (for example, sodium borohydride) in organic solvent (for example, ethanol or methanol) to afford 2.

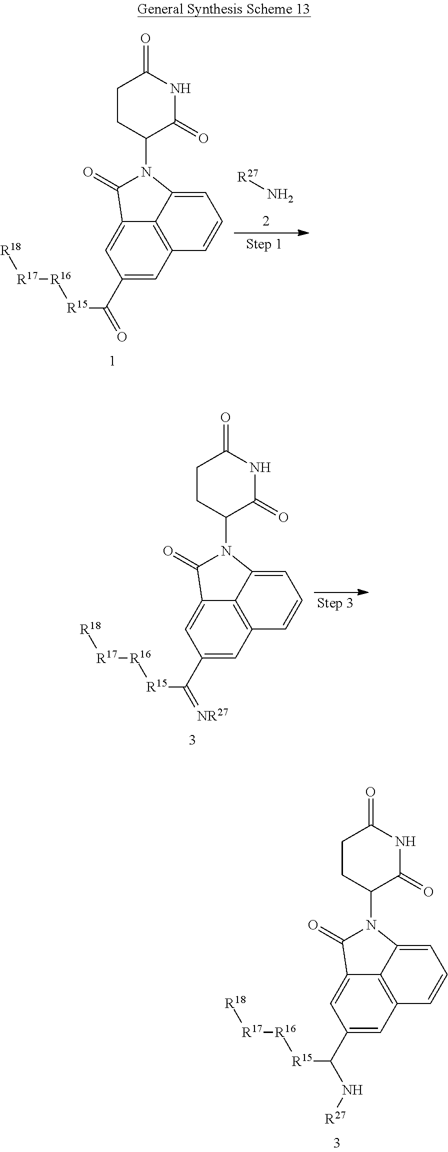

A compound of Formula XVI can be synthesized according to the route provided in General Synthesis Scheme 13. In step 1, compound 1 is reacted with 2 in the presence of a suitable desiccant (for example, molecular sieves or MgSO₄) in organic solvent (for example, dichloromethane or toluene) to afford 3. In step 2, the imine group is reduced using an appropriate reactant.

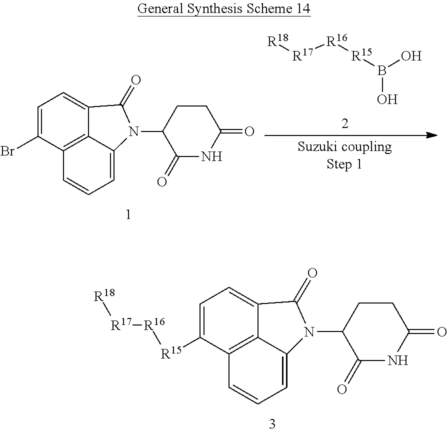

A compound of Formula XVI can be synthesized according to the route provided in General Synthesis Scheme 14. In step 1, compound 1 is reacted with 2 in the presence of a palladium catalyst (for example, Pd(OAc)₂, Pd₂dba₃, or alternatively another suitable palladium catalyst used in Suzuki coupling conditions), a ligand (for example, XPhos, PCy₃, or alternatively another suitable ligand used in Suzuki coupling conditions), and a base (for example, sodium carbonate, tribasic potassium phosphate, potassium carbonate, or alternatively another suitable base used in Suzuki coupling conditions) in aqueous organic solvent (for example, 10:1 toluene:water, 5:1 THF:water, or 1:1 ethanol:water) at elevated temperature to afford 3.

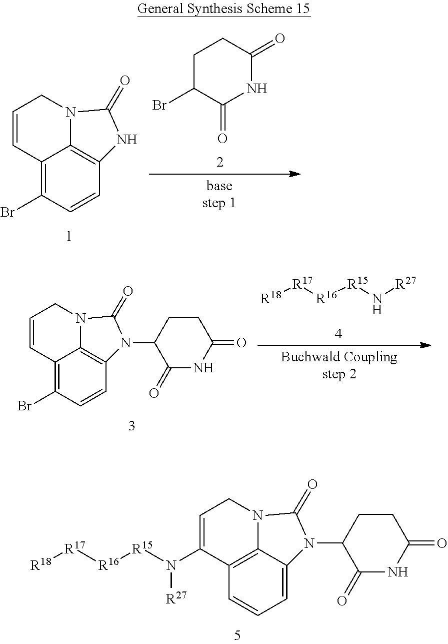

A compound of Formula XVI can be synthesized according to the route provided in General Synthesis Scheme 15. In step 1, intermediate 1 (prepared by the procedure of Saari et al. see: Saari, W. et al. “Synthesis and reactions of some dihydro and tetrahydro-4H-imidazo[5,4,1-ij]quinoline derivatives” Journal of Heterocyclic Chemistry, 1982, 19(4):837-840) is reacted with a base (for example, sodium hydride) in an organic solvent (for example, tetrahydrofuran or dichloromethane) followed by the addition of 2 to afford 3. In step 2, 3 is reacted with 4 in the presence of a palladium catalyst (for example, palladium(II) acetate, Pd₂(dba)₃, or alternatively another suitable palladium catalyst used in Buchwald-Hartwig coupling conditions), a phosphine ligand (for example, BINAP, XantPhos, or alternatively another suitable phosphine ligand used in Buchwald-Hartwig coupling conditions), and a base (for example, potassium tert-butoxide, cesium carbonate, or alternatively another suitable base used in Buchwald-Hartwig coupling conditions) in organic solvent (for example, toluene, THF, dioxane, or DMF) at elevated temperature to afford 5.

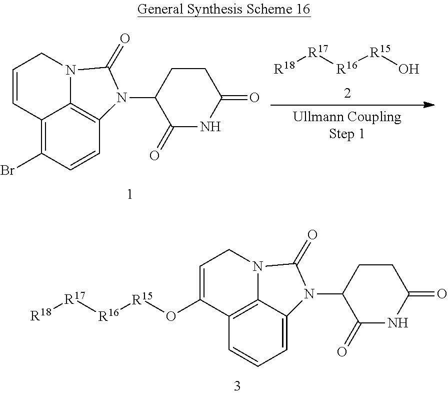

A compound of Formula XVI can be synthesized according to the route provided in General Synthesis Scheme 16. In step 1, intermediate 1 is reacted with 2 in the presence of a copper catalyst (for example, copper(I) iodide, copper(I) chloride, or alternatively another suitable copper catalyst used in Ullmann coupling conditions), a ligand (for example, bipyridine, 1,10-phenanthroline, dimethylethylenediamine, or alternatively another suitable ligand used in Ullmann coupling conditions), and a base (for example, cesium carbonate carbonate, tribasic potassium phosphate, or alternatively another suitable base used in Ullmann coupling conditions) in organic solvent (for example, dimethylsulfoxide, acetonitrile, or dioxane) at elevated temperature to afford 3.

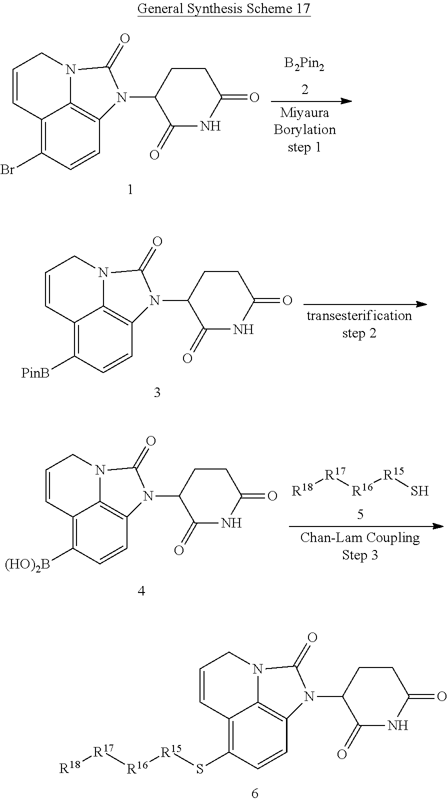

A compound of Formula XVI can be synthesized according to the route provided in General Synthesis Scheme 17. In step 1, 1 is reacted with 2 in the presence of a palladium catalyst (for example, PdCl₂(dppf), PdCl₂(PPh₃), or alternatively another suitable palladium catalyst used in Miyaura coupling conditions), a ligand (for example, XPhos, PPh₃, or alternatively another suitable ligand used in Miyaura coupling conditions), and a base (for example, potassium acetate, potassium ethoxide, potassium carbonate, or alternatively another suitable base used in Miyaura coupling conditions) in organic solvent (for example, toluene, DMA, or dioxane) at elevated temperature to afford 3. In step 2, intermediate 3 undergoes transesterification to afford 4. In step 3, intermediate 4 is reacted with 5 in the presence of a copper catalyst (for example, copper(II) bromide, copper(II) acetate, or alternatively another suitable copper catalyst used in Chan-Lam coupling conditions) and a base (for example, pyridine, 4-dimethylaminopyridine, potassium tert-butoxide, or alternatively another suitable base used in Chan-Lam coupling conditions) in organic solvent (for example, methanol, acetonitrile, or dichloromethane) under ambient air to afford 6.

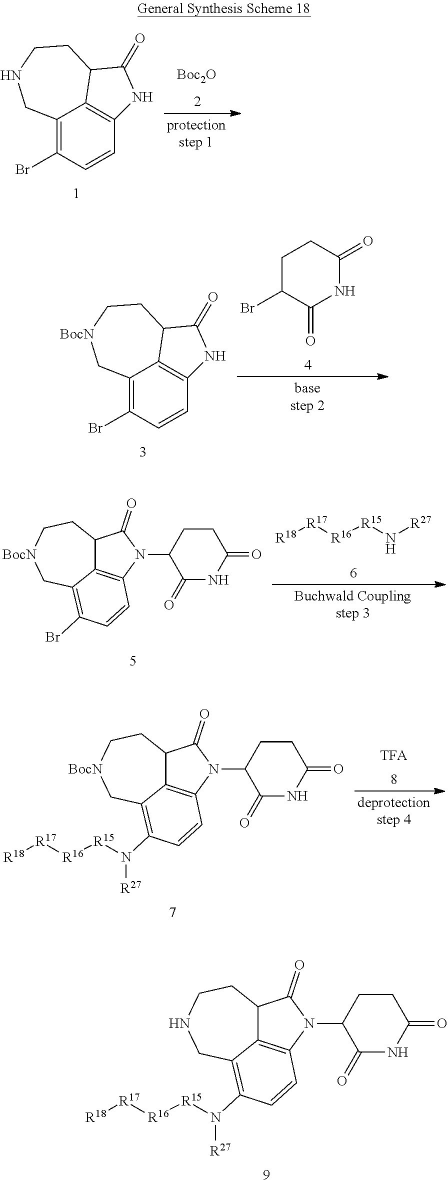

A compound of Formula XVI can be synthesized according to the route provided in General Synthesis Scheme 18. In step 1, intermediate 1 (prepared by the procedure of Kukla et al. see: Kukla, M. J. et al. “Synthesis and anti-HIV-1 activity of 4,5,6,7-tetrahydro-5-methylimidazo[4,5,1-jk][1,4]benzodiazepin-2(1H)-one (TIBO) derivatives” J. Med. Chem. 1991, 34(11):3187-3197) is reacted with 2 in the presence of a base (for example triethylamine, pyridine, or other suitable base used in Boc protection conditions) in dichloromethane to provide 3. In step 2, intermediate 3 is reacted with a base (for example sodium hydride) in an organic solvent (for example tetrahydrofuran or dichloromethane) followed by addition of 4 to provide 5. In step 3, intermediate 5 is reacted with 6 in the presence of a palladium catalyst (for example palladium(II) acetate, Pd₂(dba)₃, or other suitable palladium catalyst used in Buchwald-Hartwig coupling conditions), a phosphine ligand (for example BINAP, XantPhos, or other suitable phosphine ligand used in Buchwald-Hartwig coupling conditions), and a base (for example potassium tert-butoxide, cesium carbonate, or other suitable base used in Buchwald-Hartwig coupling conditions) in organic solvent (for example toluene, THF, dioxane, or DMF) at elevated temperature to provide 7. In step 4, intermediate 7 is reacted with 8 in dichloromethane to provide 9.

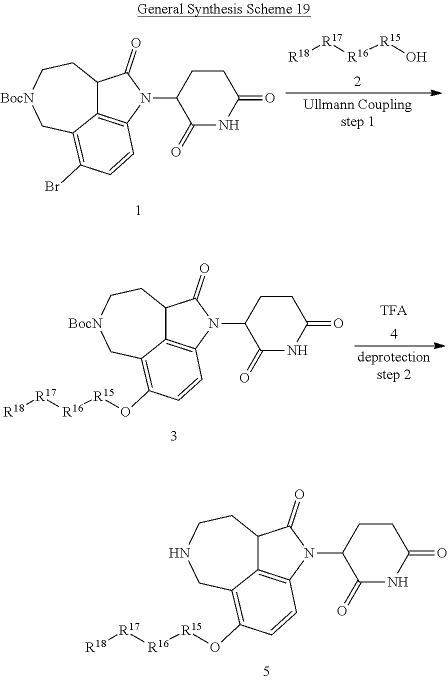

A compound of Formula XVI can be synthesized according to the route provided in General Synthesis Scheme 19. In step 1, intermediate 1 is reacted with 2 in the presence of a copper catalyst (for example, copper(I) iodide, copper(I) chloride, or alternatively another suitable copper catalyst used in Ullmann coupling conditions), a ligand (for example, bipyridine, 1,10-phenanthroline, dimethylethylenediamine, or alternatively another suitable ligand used in Ullmann coupling conditions), and a base (for example, cesium carbonate, potassium carbonate, tribasic potassium phosphate, or alternatively another suitable base used in Ullmann coupling conditions) in organic solvent (for example, dimethylsulfoxide, acetonitrile, or dioxane) at elevated temperature to afford 3. In step 2, intermediate 3 is reacted with 4 in dichloromethane to afford 5.

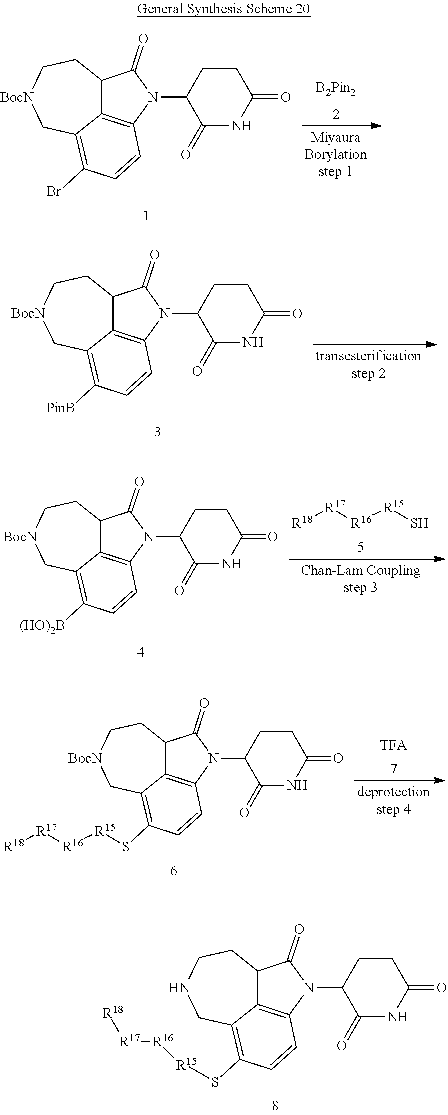

A compound of Formula XVI can be synthesized according to the route provided in General Synthesis Scheme 20. In step 1, 1 is reacted with 2 in the presence of a palladium catalyst (for example, PdCl₂(dppf), PdCl₂(PPh₃), or alternatively another suitable palladium catalyst used in Miyaura coupling conditions), a ligand (for example, XPhos, PPh₃, or alternatively another suitable ligand used in Miyaura coupling conditions), and a base (for example, potassium acetate, potassium ethoxide, potassium carbonate, or alternatively another suitable base used in Miyaura coupling conditions) in organic solvent (for example, toluene, DMA, or dioxane) at elevated temperature to afford 3. In step 2, intermediate 3 undergoes transesterification to afford 4. In step 3, intermediate 4 is reacted with 5 in the presence of a copper catalyst (for example, copper(II) bromide, copper(II) acetate, or alternatively another suitable copper catalyst used in Chan-Lam coupling conditions) and a base (for example, pyridine, 4-dimethylaminopyridine, potassium tert-butoxide, or alternatively another suitable base used in Chan-Lam coupling conditions) in organic solvent (for example, methanol, acetonitrile, or dichloromethane) under ambient air to afford 6. In step 4, 6 is reacted with 7 in dichloromethane to afford 8.

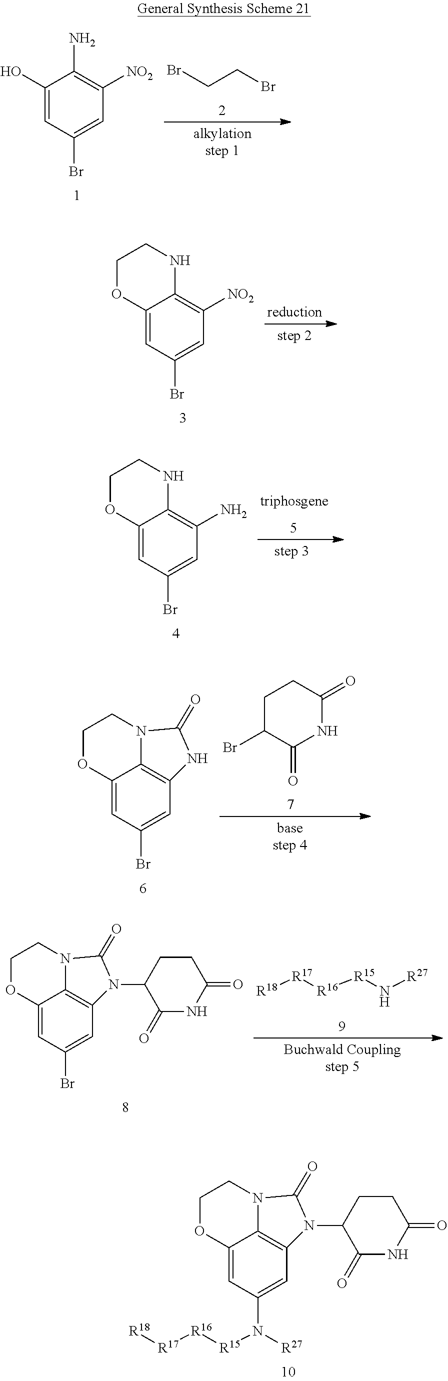

A compound of Formula XVI can be synthesized according to the route provided in General Synthesis Scheme 21. In step 1, intermediate 1 is reacted with 2 in the presence of base (for example potassium carbonate, cesium carbonate, or other suitable base used in phenol alkylation conditions) in organic solvent (for example DMF, DMA, or acetonitrile) at elevated temperature to provide 3. In step 2, intermediate 3 is reacted iron powder with HCl under aqueous conditions temperature to provide 4. In step 3, 4 is reacted with triphosgene in the presence of aluminum trichloride in dichloromethane to afford 6. In step 4, intermediate 6 is reacted with a base (for example sodium hydride) in an organic solvent (for example tetrahydrofuran or dichloromethane) followed by addition of 7 to provide 8. In step 5, 8 is reacted with 9 in the presence of a palladium catalyst (for example, palladium(II) acetate, Pd₂(dba)₃, or alternatively another suitable palladium catalyst used in Buchwald-Hartwig coupling conditions), a phosphine ligand (for example, BINAP, XantPhos, or alternatively another suitable phosphine ligand used in Buchwald-Hartwig coupling conditions), and a base (for example, potassium tert-butoxide, cesium carbonate, or alternatively another suitable base used in Buchwald-Hartwig coupling conditions) in organic solvent (for example, toluene, THF, dioxane, or DMF) at elevated temperature to afford 10.

### Example 1. Synthesis of 3-[5-(aminomethyl)-2-oxo-benzo[cd]indol-1-yl]piperidine-2,6-dione hydrochloride (Compound 1)

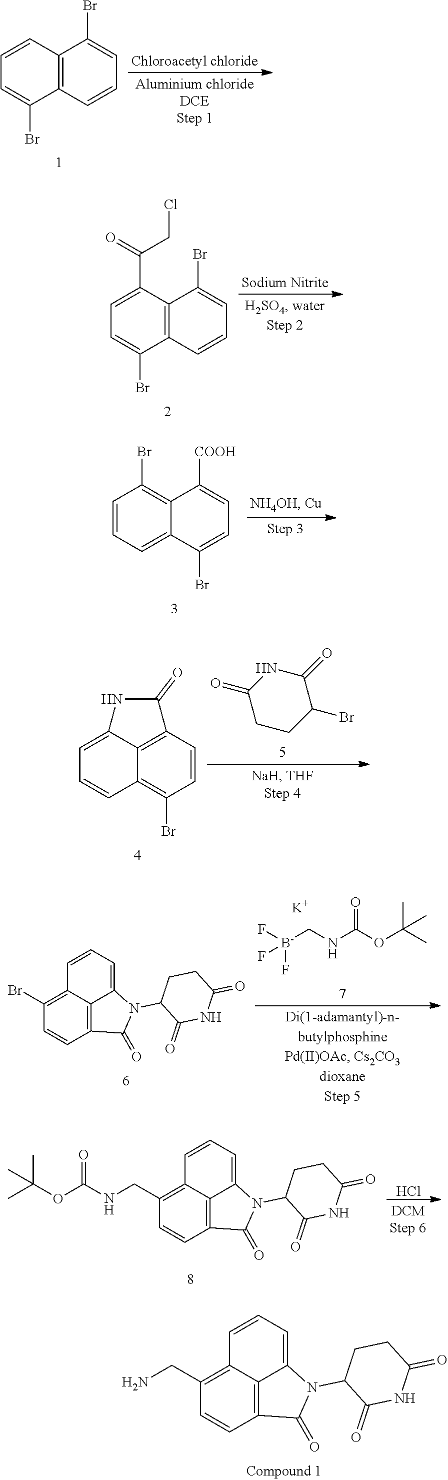

Step 1: To a stirred solution of 1,5-dibromonaphthalene 1 (120 g, 419.64 mmol) in DCE (1440 mL) was cooled to 0° C. and chloroacetyl chloride (61.61 g, 545.53 mmol, 43.39 mL) was added drop wise and the reaction mixture was stirred at this temperature for about 15 minutes. Aluminum chloride (72.74 g, 545.53 mmol, 29.81 mL) was added portion wise and the reaction mixture was slowly warmed to RT and stirred for 5 hours. The reaction mixture was quenched with cold water (500 mL) and DCM (1200 mL) then filtered through celite. The filtrate was washed with water, brine, and the DCM layer dried over anhydrous sodium sulfate, filtered, and concentrated under reduced pressure to obtain the crude solid. This crude material was stirred in 2% ethyl acetate in pet ether (1200 mL) for 30 min and the solid filtered and washed with pet ether (1200 mL) to afford 2-chloro-1-(4,8-dibromo-1-naphthyl)ethenone 2 (110 g, 294.39 mmol, 70.15% yield) as a light green solid. TLC: Rf:0.3, 10% EtOAc in Pet ether, UV detection.

Step 2: To a stirred solution of 2-chloro-1-(4,8-dibromo-1-naphthyl)ethenone 2 (200 g, 551.81 mmol) in H₂SO₄ (2400 mL) was added a solution of sodium nitrite (39.98 g, 579.40 mmol, 18.42 mL) in water (40 mL) dropwise at 0° C. and the resultant reaction mixture was stirred at 25° C. for 2 hours. The reaction mixture was then poured into cold water (870 mL) and filtered. The solid thus obtained was added to an ethyl acetate and water solution (1:1, 870:870 mL), the mixture was filtered over celite and washed with ethyl acetate (500 mL). The aqueous layer was extracted with ethyl acetate (2×100 mL). The combined organic layer was washed with brine, dried over sodium sulfate and concentrated under reduced pressure. The crude material was washed with 10% ethyl acetate in pet ether and dried to afford 4,8-dibromonaphthalene-1-carboxylic acid 3 (160 g, 402.46 mmol, 72.93% yield) as brown solid. TLC: Rf:0.2, 50% EtOAc in Pet ether, UV detection.

Step 3: To a stirred suspension of 4,8-dibromonaphthalene-1-carboxylic acid 3 (160 g, 484.89 mmol) in ammonium hydroxide (28% solution) (1.98 kg, 56.49 mol, 2.2 L), copper (8.01 g, 126.07 mmol) was added and the reaction mixture was stirred at 80° C. for 2 hours. The reaction mixture was cooled to RT and acidified with conc. hydrochloric acid to pH 2-3. The resulting suspension was filtered and dried to afford the crude product. This crude stirred in 10% ethyl acetate in pet ether for 30 min, filtered and washed with pet ether to afford 5-bromo-1H-benzo[cd]indol-2-one 4 (105 g, 342.84 mmol, 70.70% yield) as a brown solid. TLC: Rf:0.3, 70% EtOAc in Pet ether, UV detection.

Step 4: To a 500 mL three-necked round bottom flask containing a well stirred solution of 5-bromo-1H-benzo[cd]indol-2-one 4 (2.0 g, 6.85 mmol) 5-bromo-1H-benzo[cd]indol-2-one (2.0 g, 6.85 mmol) in dry THE (200 mL) was added sodium hydride (60% dispersion in mineral oil) (2.63 g, 68.53 mmol) at 0° C. and the reaction mixture was stirred at ambient temperature. After 1 hour, 3-bromopiperidine-2,6-dione 5 (6.58 g, 30.84 mmol) dissolved in dry THE (10 mL) was added at 0° C. The reaction mixture was stirred at 65° C. for 16 hours. The reaction mixture was quenched with saturated ammonium chloride solution (50 mL) then extracted with ethyl acetate (2×50 mL). Organic layers collected were dried over sodium sulphate then concentrated under reduced pressure then triturated with DCM (10 mL) to give 3-(5-bromo-2-oxo-benzo[cd]indol-1-yl)piperidine-2,6-dione 6 (1.5 g, 3.30 mmol, 48.14% yield) as a yellow color solid. LCMS (ES+): m/z 359.0 [M+H]⁺.

Step 5: To an oven dried 250 mL sealed tube was charged with 3-(5-bromo-2-oxo-benzo[cd]indol-1-yl)piperidine-2,6-dione 6 (1 g, 2.78 mmol) and potassium [[(tert-Butoxycarbonyl)amino]methyl]trifluoroborate 7 (1.65 g, 6.96 mmol) in 1,4-Dioxane (30 mL) and water (8 mL), was added cesium carbonate (2.72 g, 8.35 mmol). The contents were degassed with nitrogen gas for 10 minutes followed by addition of di(1-adamantyl)-n-butylphosphine (49.91 mg, 139.21 μmol) and palladium (II) acetate (62.51 mg, 278.42 μmol). The resulting mixture was stirred at 100° C. for 16 hours. After completion of the reaction, the reaction mixture was diluted with water (10 mL) and extracted with ethyl acetate (3×100 mL). The combined organic layers were washed with a brine solution (20 mL), dried over anhydrous sodium sulfate, filtered and concentrated in vacuo. The crude product was purified by flash column chromatography (silica gel, 230-400 mesh) eluting with 50-60% ethyl acetate-pet ether to afford tert-butyl N-[[1-(2,6-dioxo-3-piperidyl)-2-oxo-benzo[cd]indol-5-yl]methyl]carbamate 8 (200 mg, 458.73 μmol, 16.48% yield) as a pale yellow solid. LCMS (ESI): m/z 354.0 [M+H-tBu]+.

Step 6: To an oven dried 50 mL single-necked round-bottomed flask was charged with tert-butyl N-[[1-(2,6-dioxo-3-piperidyl)-2-oxo-benzo[cd]indol-5-yl]methyl]carbamate 8 (600 mg, 1.47 mmol), dissolved into DCM (10 mL), cooled to 0° C., and was added a hydrogen chloride solution 4.0 M in dioxane (4.80 g, 131.65 mmol, 6 mL). The resulting mixture was stirred at room temperature for 1 hour. The reaction mixture was concentrated in vacuo. The obtained crude product was washed with diethyl ether (20 mL) to afford 3-[5-(aminomethyl)-2-oxo-benzo[cd]indol-1-yl]piperidine-2,6-dione hydrochloride Compound 1 (505 mg, 1.40 mmol, 95.46% yield) as a pale yellow solid. LCMS (ESI): m/z 310.2 [M+H]⁺.

### Example 2. Synthesis of 3-[4-(aminomethyl)-2-oxo-benzo[cd]indol-1-yl]piperidine-2,6-dione hydrochloride (Compound 2) and 3-(4-bromo-2-oxo-benzo[cd]indol-1-yl)piperidine-2,6-dione (Compound 3)

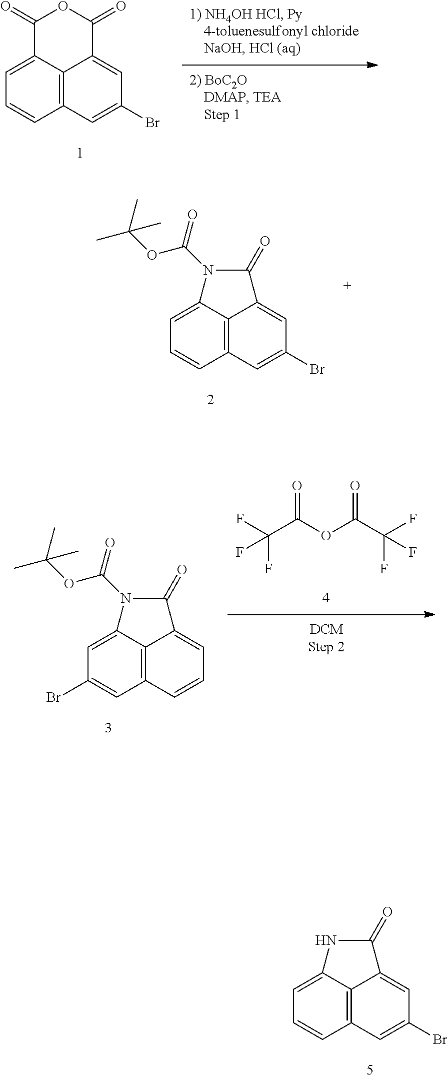 [SMILES: O=C1OC(=O)c2cc(Br)cc3cccc1c23 ; CC(C)(C)OC(=O)N1C(=O)c2cc(Br)cc3cccc1c23 ; CC(C)(C)OC(=O)N1C(=O)c2cccc3cc(Br)cc1c23 ; O=C(OC(=O)C(F)(F)F)C(F)(F)F ; O=C1Nc2cccc3cc(Br)cc1c23]

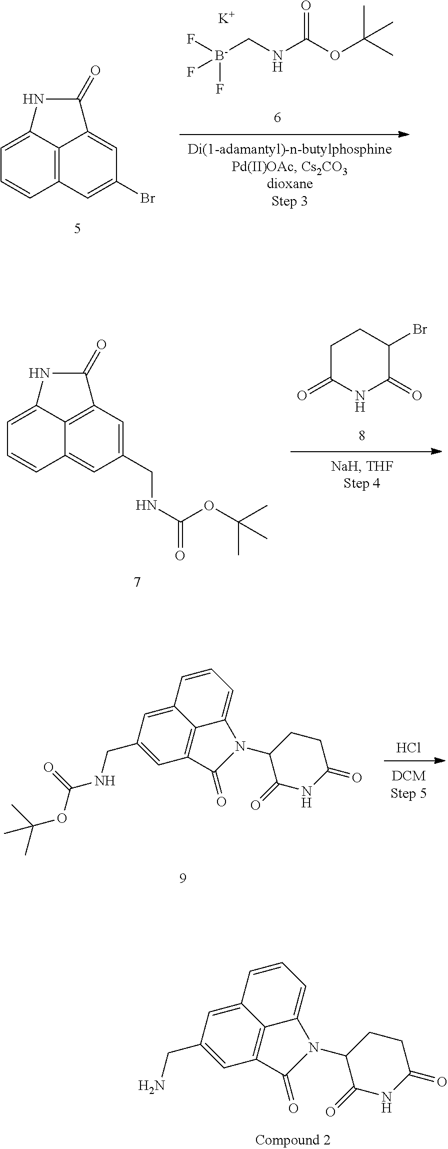

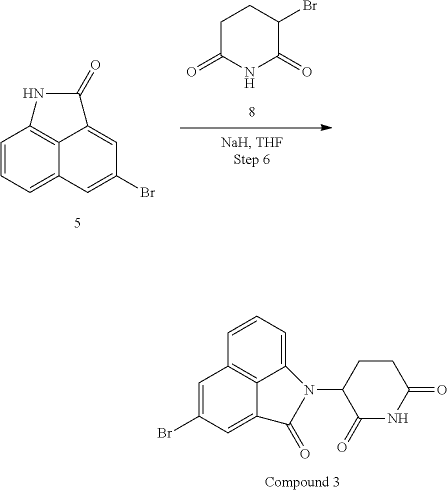 [SMILES: O=C1Nc2cccc3cc(Br)cc1c23 ; O=C1CCC(Br)C(=O)N1 ; O=C1CCC(N2C(=O)c3cc(Br)cc4cccc2c34)C(=O)N1]

Step 1 part (1): To a solution of 7-bromo-14-oxatricyclotrideca-,2(6),3(7),4(8),5(9)-pentaene-10,11-dione 1 (CAS #24050-49-5, 5 g, 18.05 mmol) and hydroxylamine hydrochloride (1.25 g, 18.05 mmol, 750.92 μL) in pyridine (36 mL) was conducted under reflux for 5 hours, followed by cooling to 80° C. Then 4-toluenesulfonyl chloride (6.88 g, 36.09 mmol) was added to the reaction system. After addition, the temperature was raised and the reaction was stirred at reflux for 5 h, followed by cooling. The reaction mixture was poured into 90 mL of water and stirred to precipitate crystals, which were collected by filtration. The crystals were transferred to a beaker and washed successively with 90 mL of a NaHCO₃ aqueous solution and 90 mL of water, followed by filtration. The crystals were washed with water and dried to give an intermediate for further reaction. The whole amount of the intermediate, was dissolved in EtOH (15 mL) and water (18 mL) were put in a reactor and stirred. Then sodium hydroxide, flake, 98% (1.4 M, 60 mL) was added dropwise to the mixture. Thereafter, the mixture was heated to refluxing temperature, at which the reaction was carried out for 3 hours while distilling off ethanol. After completion of the reaction, the reaction mixture was cooled to 75° C., and hydrochloric acid, 36% w/w aq. soln. (8.00 g, 219.41 mmol, 10 mL) was added dropwise. In the meantime, crystals precipitated at 60° C. After completion of the dropwise addition, the mixture was further cooled. The precipitated crystals were collected by filtration, washed water, and dried to afford a regioisomer mixture of 4-bromo-1H-benzo[cd]indol-2-one and 7-bromo-1H-benzo[cd]indol-2-one as a yellow solid. Used in the next step without further purification.

Step 1 part (2): To a stirred solution of 4-bromo-1H-benzo[cd]indol-2-one and 7-bromo-1H-benzo[cd]indol-2-one (3 g, 12.1 mmol) (regio-isomer mixture) in DCM (30 mL) was added N,N-diethylethanamine (1.84 g, 18.14 mmol, 2.53 mL) and N,N-dimethylpyridin-4-amine (73.87 mg, 604.66 μmol) at RT, followed by the addition of tert-butoxycarbonyl tert-butyl carbonate (1.98 g, 9.07 mmol, 2.08 mL) at 0° C., the cooling bath was removed and the reaction mixture stirred at RT for 3 hours. The reaction mixture was poured into water and extracted with DCM, dried over sodium sulfate, filtered and solvent removed under reduced pressure. The crude compound was purified by column chromatography (silica gel; 4% ethyl acetate-pet ether) to give tert-butyl 4-bromo-2-oxo-benzo[cd]indole-1-carboxylate 2 (1 g, 2.77 mmol, 45.87% yield) as an off white solid and tert-butyl 7-bromo-2-oxo-benzo[cd]indole-1-carboxylate 3 (1.1 g, 1.88 mmol, 31.07% yield) as an off white solid.

Step 2: To the stirred solution of tert-butyl 4-bromo-2-oxo-benzo[cd]indole-1-carboxylate 2 (2.0 g, 5.74 mmol) in DCM (15 mL) was added (2,2,2-trifluoroacetyl) 2,2,2-trifluoroacetate 4 (12.06 g, 57.44 mmol, 8.10 mL) over a period of 5 minutes at 0° C. The reaction mixture was warmed to room temperature and stirred at this temperature for 3 hours. The reaction mixture was concentrated under reduced pressure at 45° C. The crude product was triturated using diethyl ether to afford desired product of 4-bromo-1H-benzo[cd]indol-2-one 5 (1.9 g, 7.66 mmol, 133.34% yield) as greenish liquid. The crude product was taken for next step without any further purification.

Step 3: To a 250 mL sealed tube containing a well-stirred suspension of 4-bromo-1H-benzo[cd]indol-2-one 5 (1.5 g, 6.05 mmol), potassium; (tert-butoxycarbonylamino)methyl-trifluoro-boranuide 6 (3.58 g, 15.12 mmol) in 1,4-dioxane (45 mL), water (15 mL) was added cesium carbonate (5.91 g, 18.14 mmol), Di(1-adamantyl)-n-butylphosphine (108.40 mg, 302.33 μmol) and palladium (II) acetate (135.75 mg, 604.66 μmol) at ambient temperature under nitrogen. The resulted mixture was stirred at 100° C. for 16 hours. The reaction mixture was cooled to ambient temperature, quenched with water (5 mL), extracted with ethyl acetate (3×60 mL), the combined organic layer was washed with brine (30 mL), dried over anhydrous sodium sulphate, filtered and concentrated under reduced pressure to give a crude residue. The crude compound was purified by flash column chromatography (silica gel, 230-400 mesh) eluting with 50-60% ethyl acetate in petroleum ether to give tert-butyl N-[(2-oxo-1H-benzo[cd]indol-4-yl)methyl]carbamate 7 (1.3 g, 4.05 mmol, 67.02% yield) as pale yellow solid. LC-MS (ESI) m/z: 243.2 [M-tBu+H]⁺.

Step 4: To a 500 mL three-necked round bottom flask containing a well-stirred suspension of tert-butyl N-[(2-oxo-1H-benzo[cd]indol-4-yl)methyl]carbamate 7 (2.6 g, 8.72 mmol) in tetrahydrofuran (150 mL) was added sodium hydride (60% dispersion in mineral oil) (2.58 g, 64.49 mmol) at 0° C. under nitrogen. The reaction mixture was allowed to stir at ambient temperature for 1 hour. To the reaction mixture was added 3-bromopiperidine-2,6-dione 8 (5.35 g, 27.89 mmol) in tetrahydrofuran (15 mL) at 0° C. The reaction mixture was stirred at 65° C. for 4 hours. The reaction mixture was cooled to 0° C., quenched with saturated ammonium chloride solution (30 mL), extracted with ethyl acetate (3×150 mL), the combined organic layer was dried over anhydrous sodium sulphate, filtered and concentrated under reduced pressure to get crude residue. The crude compound was purified by column chromatography (Silica gel, 230-400 mesh) eluting with 40-60% ethyl acetate in petroleum ether to get tert-butyl N-[[1-(2,6-dioxo-3-piperidyl)-2-oxo-benzo[cd]indol-4-yl]methyl]carbamate 9 (2.6 g, 5.91 mmol, 67.76% yield) as yellow solid. LC-MS (ESI) m/z: 408.0 [M−H]—.

Step 5: To a 100 mL round bottom flask containing well stirred solution of tert-butyl N-[[1-(2,6-dioxo-3-piperidyl)-2-oxo-benzo[cd]indol-4-yl]methyl]carbamate 9 (1 g, 2.44 mmol) in DCM (10 mL) was added 4M HCl in 1,4-dioxane (89.05 mg, 2.44 mmol, 10 mL) dropwise at 0° C. The cooling bath was removed and the reaction mixture stirred at room temperature for 2 hours. The reaction mixture was concentrated in vacuo to give crude material which was triturated with diethyl ether (10 mL) and dried to afford 3-[4-(aminomethyl)-2-oxo-benzo[cd]indol-1-yl]piperidine-2,6-dione hydrochloride Compound 2 (800 mg, 2.17 mmol, 89.04% yield) LCMS (ESI): m/z 310.2 [M+H]⁺.

Step 6: To a stirred solution of 4-bromo-1H-benzo[cd]indol-2-one 2 (5 g, 20.16 mmol) in THF (50 mL) was added sodium hydride (4.84 g, 201.55 mmol) at 0° C. under nitrogen atmosphere. Reaction mixture was stirred for 2 hours at room temperature. The reaction mixture was cooled 0° C., 3-bromopiperidine-2,6-dione 8 (19.35 g, 100.78 mmol) was added in portions at 0° C. under nitrogen atmosphere, then the reaction mixture was heated to 65° C. and stirred at this temperature for 2 hours at 65° C. Water (100 mL) water and EtOAc (10V, 50 mL) was added and the layers separated, the aqueous layer was extracted with EtOAc (50 mL). Combined organic layers were washed with brine solution (25 mL), dried over sodium sulphate and concentrated under reduced pressure to afford 3-(4-bromo-2-oxo-benzo[cd]indol-1-yl)piperidine-2,6-dione Compound 3 (3.0 g, 7.59 mmol, 37.66% yield).

### Example 3. Synthesis of Afford 3-(6-bromo-2-oxopyrrolo[4,3,2-de]quinolin-1(2H)-yl)piperidine-2,6-dione (Compound 4)

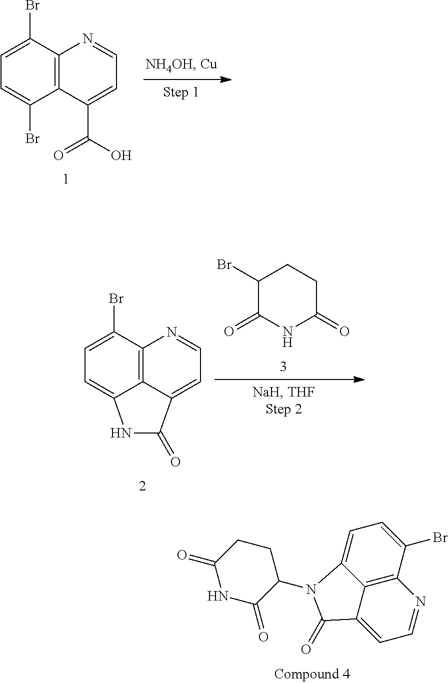

Step 1: To a stirred suspension of 5,8-dibromoquinoline-4-carboxylic acid 1 (CAS: 1603199-45-6) in ammonium hydroxide (28% solution) (100 eq), copper (4 eq) was added and the reaction mixture was stirred at 80° C. for 2 hours. The reaction mixture was cooled to RT and worked up and purified using standard protocols to afford 6-bromopyrrolo[4,3,2-de]quinolin-2(1H)-one 2, as the product.

Step 2: To a solution of 6-bromopyrrolo[4,3,2-de]quinolin-2(1H)-one 2 in THE (10 vol eq) at 0° C. is added NaH (5 eq) and stirred at this temp for 15 min before the addition of 3-bromopiperidine-2,6-dione 3 (1 eq). The reaction mixture is slowly heated to 60° C. and stirred at this temperature until completion of the reaction. A standard workup and purification using standard protocols to afford 3-(6-bromo-2-oxopyrrolo[4,3,2-de]quinolin-1(2H)-yl)piperidine-2,6-dione Compound 4.

### Example 4. Synthesis of 3-(8-bromo-5-oxopyrrolo[2,3,4-de]quinolin-4(5H)-yl)piperidine-2,6-dione (Compound 5)

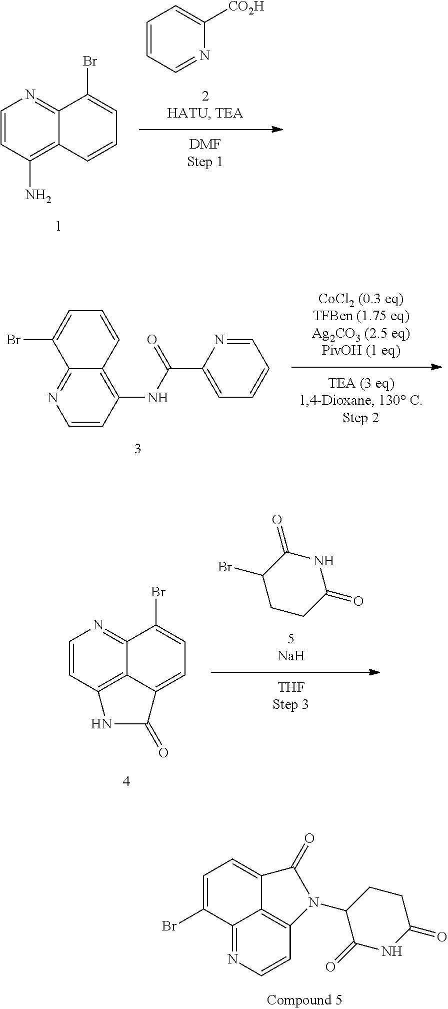 [SMILES: Nc1ccnc2c(Br)cccc12 ; O=C(O)c1ccccn1 ; O=C(Nc1ccnc2c(Br)cccc12)c1ccccn1 ; O=C1Nc2ccnc3c(Br)ccc1c23 ; O=C1CCC(Br)C(=O)N1 ; O=C1CCC(N2C(=O)c3ccc(Br)c4nccc2c34)C(=O)N1]

Step 1: To a stirred suspension of 8-bromoquinolin-4-amine 1 (CAS: 65340-75-2) in DMF (10 vol eq) was added Picolinic acid 2 (1 eq), TEA (3 eq) followed by HATU (1.1 eq) and the mixture was stirred at room temperature. Upon completion of reaction the mixture is quenched, worked up and purified using standard protocols to afford N-(8-bromoquinolin-4-yl)picolinamide 3.

Step 2: To a suspension of N-(8-bromoquinolin-4-yl)picolinamide 3 (1 eq), CoCl₂ (0.3 eq), Ag₂CO₃ (2.5 eq), Benzene-1,3,5-triyl Triformate (TFBen, 1.75 eq), PivOH (1 eq) and TEA (3 eq) in 1,4-dioxane (10 vol eq) is heated at 130° C. for 20 h. Upon reaction completion the mixture is worked up and purified using standard protocols to afford 8-bromopyrrolo[2,3,4-de]quinolin-5(4H)-one 4. (According to procedures from Org. Lett. 2019, 21, 5694-5698.)

Step 3: To a 8-bromopyrrolo[2,3,4-de]quinolin-5(4H)-one 4 in THE (10 vol eq) at 0° C. is added NaH (5 eq) and stirred at this temp for 15 min before the addition of 3-bromopiperidine-2,6-dione 5 (1 eq). The reaction mixture is slowly heated to 60° C., and stirred at this temperature until completion of the reaction. A standard workup and purification using standard protocols to afford 3-(8-bromo-5-oxopyrrolo[2,3,4-de]quinolin-4(5H)-yl)piperidine-2,6-dione Compound 12.

### Example 5. Synthesis of 3-(5-bromo-2-oxopyrrolo[2,3,4-de]isoquinolin-1(2H)-yl)piperidine-2,6-dione (Compound 6)

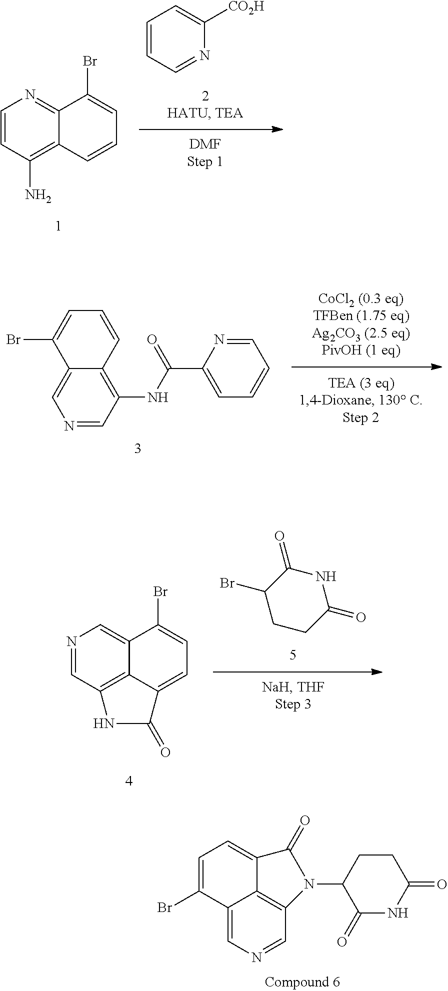 [SMILES: Nc1ccnc2c(Br)cccc12 ; O=C(O)c1ccccn1 ; O=C(Nc1cncc2c(Br)cccc12)c1ccccn1 ; O=C1Nc2cncc3c(Br)ccc1c23 ; O=C1CCC(Br)C(=O)N1 ; O=C1CCC(N2C(=O)c3ccc(Br)c4cncc2c34)C(=O)N1]

Step 1: To a stirred suspension of 8-bromoisoquinolin-4-amine 1 (CAS: 1781091-48-2) in DMF (10 vol eq) was added Picolinic acid 2 (1 eq), TEA (3 eq) followed by HATU (1.1 eq) and the mixture was stirred at room temperature. Upon completion of reaction the mixture is quenched, worked up and purified using standard protocols to afford N-(8-bromoisoquinolin-4-yl)picolinamide 3.

Step 2: To a suspension of N-(8-bromoisoquinolin-4-yl)picolinamide 3 (1 eq), CoCl₂ (0.3 eq), Ag₂CO₃ (2.5 eq), Benzene-1,3,5-triyl Triformate (TFBen, 1.75 eq), PivOH (1 eq) and TEA (3 eq) in 1,4-dioxane (10 vol eq) is heated at 130° C. for 20 hours. Upon reaction completion the mixture is worked up and purified using standard protocols to afford 5-bromopyrrolo[2,3,4-de]isoquinolin-2(1H)-one 4. (According to procedures from Org. Lett. 2019, 21, 5694-5698.)

Step 3: To a 5-bromopyrrolo[2,3,4-de]isoquinolin-2(1H)-one 4 in THE (10 vol eq) at 0° C. is added NaH (5 eq) and stirred at this temp for 15 min before the addition of 3-bromopiperidine-2,6-dione 5 (1 eq). The reaction mixture is slowly heated to 60° C., and stirred at this temperature until completion of the reaction. A standard workup and purification using standard protocols to afford 3-(5-bromo-2-oxopyrrolo[2,3,4-de]isoquinolin-1(2H)-yl)piperidine-2,6-dione Compound 13.

### Example 6. Synthesis of 3-(6-bromo-2-oxopyrrolo[4,3,2-de]isoquinolin-1(2H)-yl)piperidine-2,6-dione (Compound 7)

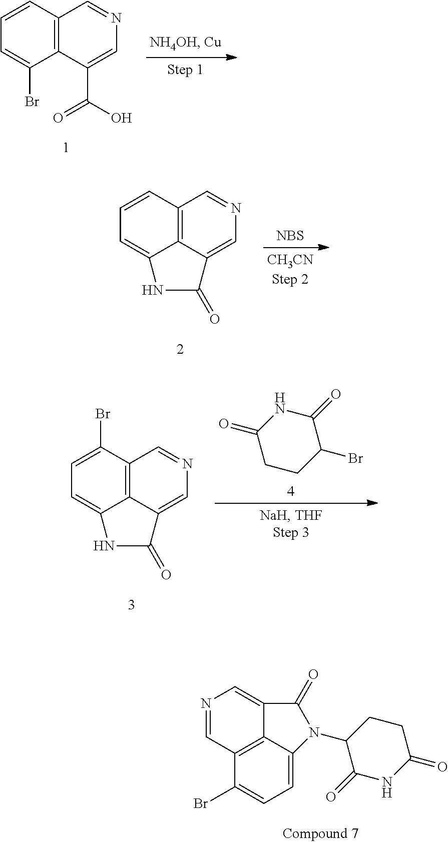

Step 1: To a stirred suspension of 5-bromoisoquinoline-4-carboxylic acid 1 (WO2012090177A2, 1 eq) in ammonium hydroxide (28% solution) (100 eq), copper (4 eq) was added and the reaction mixture was stirred at 80° C. for 2 hours. The reaction mixture was cooled to RT and worked up and purified using standard protocols to afford pyrrolo[4,3,2-de]isoquinolin-2(1H)-one 2.

Step 2: To a solution of pyrrolo[4,3,2-de]isoquinolin-2(1H)-one 2 (1 eq) in CH₃CN (10 vol) at 0° C. is added NBS (1 eq), the cooling bath is removed and the reaction mixture stirred at room temperature for 16 hours. A standard workup and purification using standard protocols to afford 6-bromopyrrolo[4,3,2-de]isoquinolin-2(1H)-one 3.

Step 3: To a solution 6-bromopyrrolo[4,3,2-de]isoquinolin-2(1H)-one (1 eq) in THE (10 vol eq) at 0° C. is added NaH (60% in mineral oil, 5 eq) and stirred at this temperature for 15 min before the addition of 3-bromopiperidine-2,6-dione 4 (1 eq). The reaction mixture is slowly heated to 60° C. and stirred at this temperature until completion of the reaction. A standard workup and purification using standard protocols to afford 3-(6-bromo-2-oxopyrrolo[4,3,2-de]isoquinolin-1(2H)-yl)piperidine-2,6-dione Compound 7.

### Example 7. Synthesis of 3-(6-bromo-2-oxopyrrolo[2,3,4-ij]isoquinolin-1(2H)-yl)piperidine-2,6-dione (Compound 8)

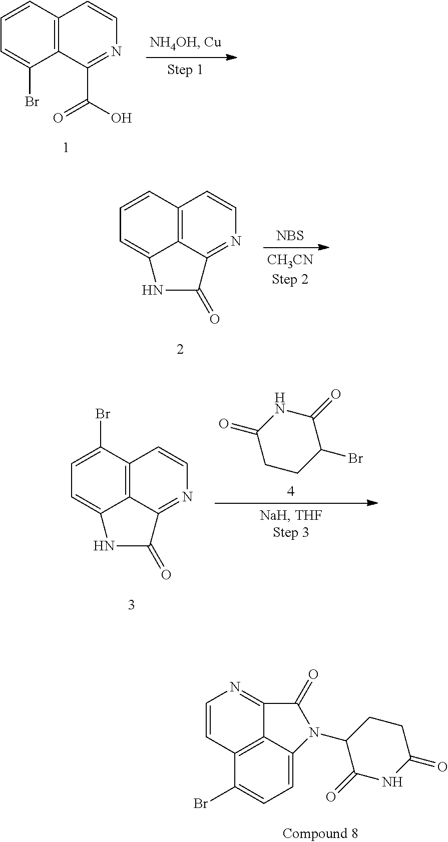

Step 1: To a stirred suspension of 8-bromoisoquinoline-1-carboxylic acid 1 (CAS #: 1256818-87-7, 1 eq) in ammonium hydroxide (28% solution) (100 eq), copper (4 eq) was added and the reaction mixture was stirred at 80° C. for 2 hr. The reaction mixture was cooled to RT and worked up and purified using standard protocols to afford pyrrolo[2,3,4-ij]isoquinolin-2(1H)-one 2.

Step 2: To a solution of pyrrolo[2,3,4-ij]isoquinolin-2(1H)-one 2 (1 eq) in CH₃CN (10 vol) at 0° C. is added NBS (1 eq), the cooling bath is removed and the reaction mixture stirred at room temperature for 16 hours. A standard workup and purification using standard protocols to afford 6-bromopyrrolo[2,3,4-ij]isoquinolin-2(1H)-one 3.

Step 3: To a solution 6-bromopyrrolo[2,3,4-ij]isoquinolin-2(1H)-one 3 (1 eq) in THE (10 vol eq) at 0° C. is added NaH (60% in mineral oil, 5 eq) and stirred at this temperature for 15 min before the addition of 3-bromopiperidine-2,6-dione 4 (1 eq). The reaction mixture is slowly heated to 60° C. and stirred at this temperature until completion of the reaction. A standard workup and purification using standard protocols to afford 3-(6-bromo-2-oxopyrrolo[2,3,4-ij]isoquinolin-1(2H)-yl)piperidine-2,6-dione Compound 8.

### Example 8. Synthesis of 3-(3-bromo-8-oxopyrrolo[4,3,2-de]phthalazin-7(8H)-yl)piperidine-2,6-dione (Compound 9)

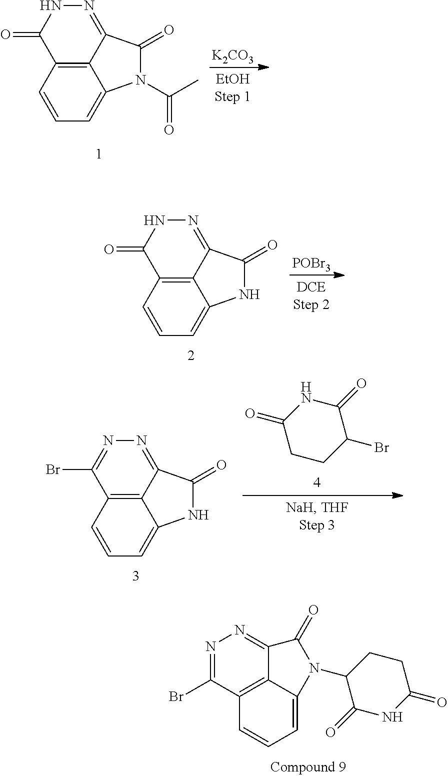

Step 1: To a solution of 7-acetyl-2,7-dihydropyrrolo[4,3,2-de]phthalazine-3,8-dione 1 (Heterocycles (1981), 16(1), 21-4, 1 eq) in EtOH (10 vol eq) is added potassium carbonate (3 eq) and the reaction mixture stirred from room temperature to 50° C. Standard workup and purification using standard protocols to afford 2,7-dihydropyrrolo[4,3,2-de]phthalazine-3,8-dione 2.

Step 2: To a solution of 2,7-dihydropyrrolo[4,3,2-de]phthalazine-3,8-dione 2 in DCE (10 vol eq) is added POBr₃ (1 eq) and the reaction stirred at 90° C. for 16 hours. A standard workup and purification using standard protocols to afford 3-bromopyrrolo[4,3,2-de]phthalazin-8(7H)-one 3.

Step 3: To a solution of 3-bromopyrrolo[4,3,2-de]phthalazin-8(7H)-one 3 (1 eq) in THE (10 vol eq) at 0° C. is added NaH (5 eq) and stirred at this temp for 15 min before the addition of 3-bromopiperidine-2,6-dione 4 (1 eq). The reaction mixture is slowly heated to 60° C., and stirred at this temperature until completion of the reaction. A standard workup and purification using standard protocols to afford 3-(3-bromo-8-oxopyrrolo[4,3,2-de]phthalazin-7(8H)-yl)piperidine-2,6-dione Compound 9.

### Example 9. Synthesis of 3-(6-bromo-2-oxopyrrolo[4,3,2-de]quinazolin-1(2H)-yl)piperidine-2,6-dione (Compound 10)

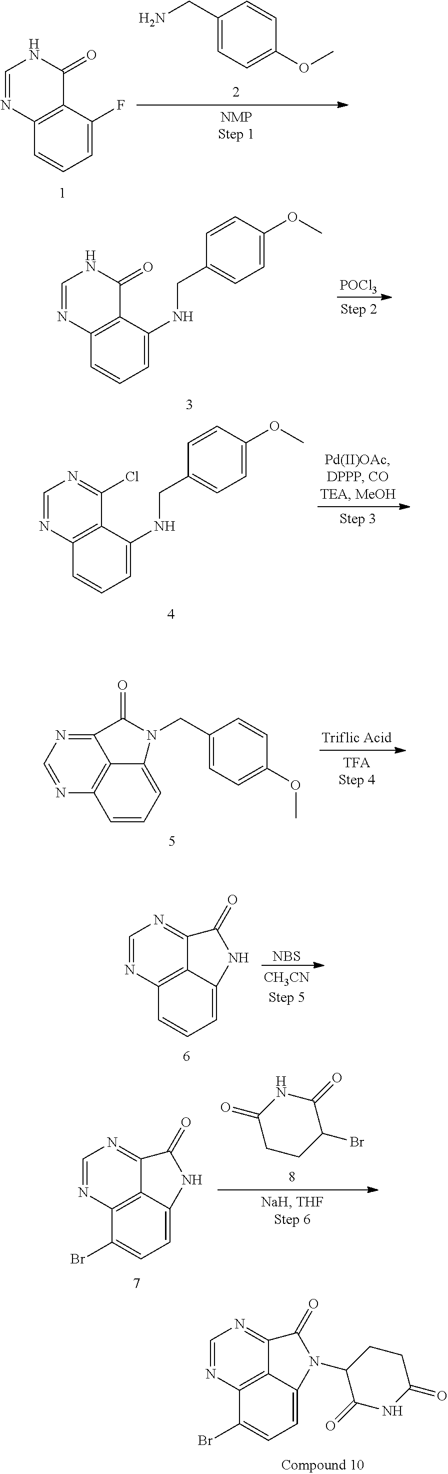

Step 1: To a solution of 5-fluoro-4(1H)-quinazolinone 1 (CAS #436-72-6, 1 eq) and 4-methylbenzylamine 2 (5 eq) in NMP is heated 100° C. until completion of the reaction. A standard work up and purification using standard protocols to afford 5-((4-methoxybenzyl)amino)quinazolin-4(3H)-one 3.

Step 2: To a solution of 5-((4-methoxybenzyl)amino)quinazolin-4(3H)-one 3 (1 eq) in a toluene (10 vol eq) is added POCl₃ (1 eq) and the reaction mixture is heated to 100° C. until completion of the reaction. A standard workup and purification using standard protocols to afford 4-chloro-N-(4-methoxybenzyl)quinazolin-5-amine 4.

Step 3: To a solution of 4-chloro-N-(4-methoxybenzyl)quinazolin-5-amine 4 (1 ea) in MeOH (10 vol eq) was added TEA (4 eq) then the solution is purged with argon for 10 min. DPPP (0.2 eq) and palladium (II) acetate (0.1 eq) is added and the reaction mixture shaken in a parr-autoclave at 100° C. under an atmosphere of 70 Psi of carbon monoxide until the reaction is deemed complete. A standard workup and purification using standard protocols to afford 1-(4-methoxybenzyl)pyrrolo[4,3,2-de]quinazolin-2(1H)-one 5.

Step 4: To a cooled solution of produce 1-(4-methoxybenzyl)pyrrolo[4,3,2-de]quinazolin-2(1H)-one 5 in TFA (12 vol eq) is added triflic acid (8 eq) and the reaction mixture is stirred at room temperature until the reaction is complete. A standard workup and purification using standard protocols to afford pyrrolo[4,3,2-de]quinazolin-2(1H)-one 6.

Step 5: To a mixture of pyrrolo[4,3,2-de]quinazolin-2(1H)-one 6 (1 eq) in CH₃CN (10 vol eq) was added NBS (1 eq) at 0° C., the cooling bath removed and the reaction mixture stirred at room temperature until the reaction is deemed complete. A standard workup and purification using standard protocols to afford 6-bromopyrrolo[4,3,2-de]quinazolin-2(1H)-one 7.

Step 6: To a solution of 6-bromopyrrolo[4,3,2-de]quinazolin-2(1H)-one 7 (1 eq) in THE (10 vol eq) at 0° C. is added NaH (5 eq) and stirred at this temp for 15 min before the addition of 3-bromopiperidine-2,6-dione (1 eq). The reaction mixture is slowly heated to 60° C., and stirred at this temperature until completion of the reaction. A standard workup and purification using standard protocols to afford 3-(6-bromo-2-oxopyrrolo[4,3,2-de]quinazolin-1(2H)-yl)piperidine-2,6-dione Compound 10.

### Example 10. Synthesis of 3-(8-bromo-5-oxopyrrolo[2,3,4-de]quinazolin-4(5H)-yl)piperidine-2,6-dione (Compound 11)

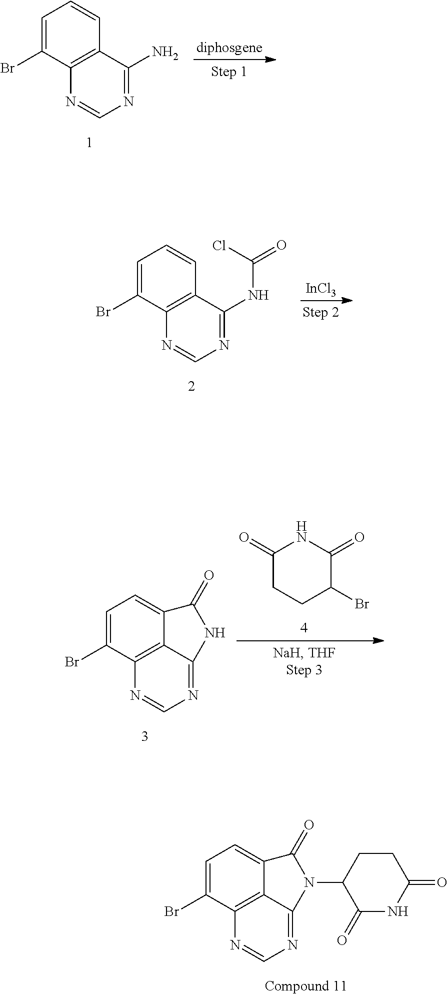

Step 1: To a solution of 8-bromo-4-quinazolinamine 1 (CAS #1260657-19-9, 1 eq) in dichloroethane: pyridine (10:1) at 0° C. is added diphosgene (1.1-1.5 eq) and the reaction stirred at this temp for 2 hours, followed by slowly increasing the temperature to 50° C., then maintain at this temperature for 2 hours. The reaction mixture is quenched with 1N HCl and standard work up and purification to afford (8-bromoquinazolin-4-yl)carbamic chloride 2.

Step 2: To a solution of (8-bromoquinazolin-4-yl)carbamic chloride 2 in dichloroethane at 0° C. is added indium trichoride (1.1-5 eq), and the reaction mixture heated to reflux, and maintained at this temperature until completion of the reaction. The cooled reaction mixture is then subject to a standard work up and purification to afford 8-bromopyrrolo[2,3,4-de]quinazolin-5(4H)-one 3.

Step 3: To a solution of 8-bromopyrrolo[2,3,4-de]quinazolin-5(4H)-one 3 in THE at 0° C. is added NaH (60% dispersion in mineral oil, 10-15 eq) in portions, the cooing bath is removed and the reaction mixture is stirred at this temperature for 1 hours. The reaction mixture is cooled to 0° C., 3-bromo-glutarimide 4 (5-8 eq) is added in portions, the cooling bath removed, and slowly heated to 70° C. until the reaction is judged complete. Standard workup and purification using standard protocols to afford 3-(8-bromo-5-oxopyrrolo[2,3,4-de]quinazolin-4(5H)-yl)piperidine-2,6-dione Compound 11.

### Example 11. Synthesis of 3-(3-bromo-2-methyl-7-oxo-2,7-dihydro-6H-pyrrolo[4,3,2-cd]indazol-6-yl)piperidine-2,6-dione (Compound 12) and 3-(3-bromo-2-methyl-6-oxo-2,6-dihydro-7H-pyrrolo[2,3,4-cd]indazol-7-yl)piperidine-2,6-dione (Compound 13)

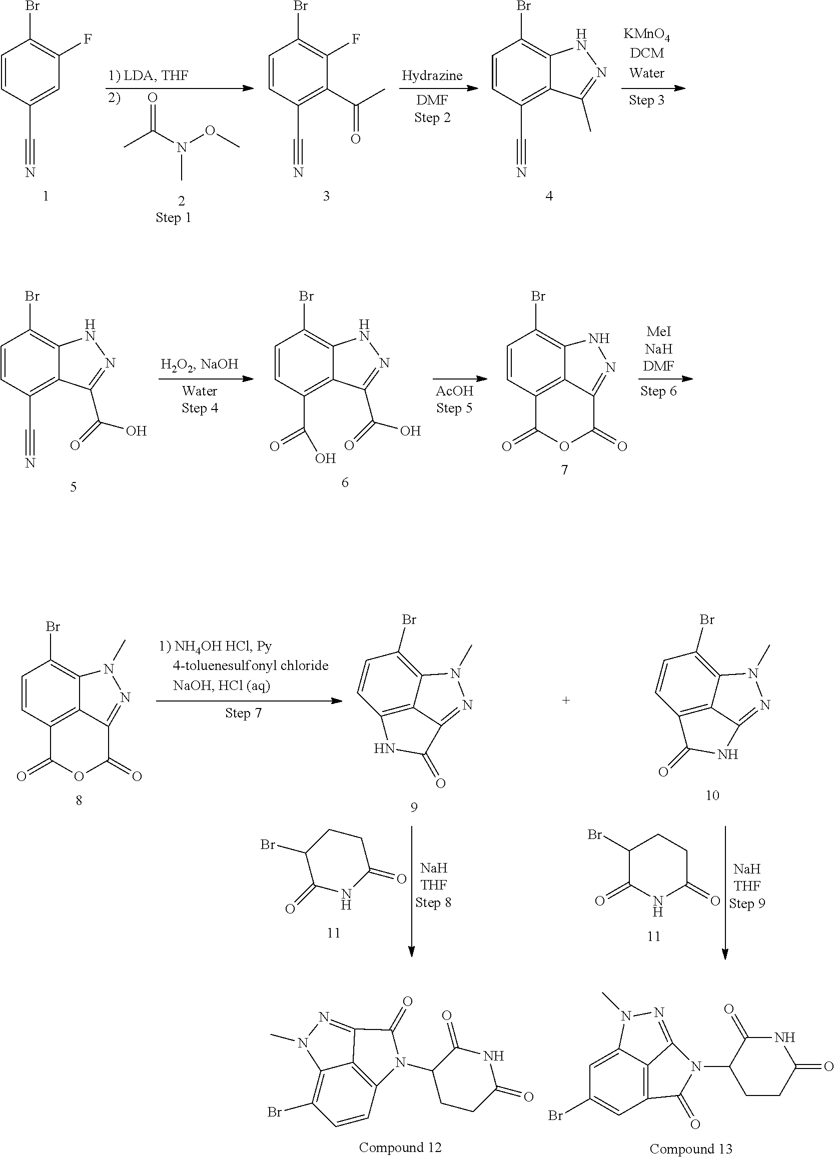

Step 1: To a solution of commercially available 4-bromo-3-fluorobenzonitrile 1 (Cas #: 133059-44-6, 1 eq.) in THE at −78° C. would be added a dropwise solution of LDA (2M in THF, 1.1 eq) and stirred at this temperature for 1-3 hours. At this time a solution of N-methoxy-N-methylacetamide 2 (1.2 eq) in THE is added dropwise, the cooling bath is removed, and the reaction mixture is stirred for 1-24 additional hours. Isolation and purification used standard procedures to afford 2-acetyl-4-bromo-3-fluorobenzonitrile 3.

Step 2: To a solution of 2-acetyl-4-bromo-3-fluorobenzonitrile 3 (1 eq), in a DMF at 0° C. is added hydrazine (1.1 eq) dropwise, the cooling bath is removed and the reaction is allowed to stir at RM temperature for an additional 1-24 hours. Isolation and purification used standard procedures to afford 7-bromo-3-methyl-1H-indazole-4-carbonitrile 4.

Step 3: To a solution of 7-bromo-3-methyl-1H-indazole-4-carbonitrile 4 (1 eq) in a mixture of DCM and water is added KMnO₄ (10 eq) and stirred at room temperature to reflux for 1-24 hours. Isolation and purification used standard protocols to afford 7-bromo-4-cyano-1H-indazole-3-carboxylic acid 5.

Step 4: To a solution of 7-bromo-4-cyano-1H-indazole-3-carboxylic acid 5 (1 eq) in 4:1 water: hydrogen peroxide is added 20 eq of NaOH and the reaction mixture refluxed for 1-24 hours. Isolation and purification used standard protocols to afford 7-bromo-1H-indazole-3,4-dicarboxylic acid 6.

Step 5: To a mixture of 7-bromo-1H-indazole-3,4-dicarboxylic acid 6 (1 eq) in AcOH (10 vol eq.) is heated at 100° C. until completion of the reaction. Standard workup and purification protocols to afford 8-bromo-3H-pyrano[3,4,5-cd]indazole-3,5(1H)-dione 7.

Step 6: To a cooled solution of 8-bromo-3H-pyrano[3,4,5-cd]indazole-3,5(1H)-dione 7 (1 eq) in DMF is added NaH (60% in oil, 2 eq) and the reaction mixture stirred at this temp for 10 min before the addition of Mel (1.1 eq). The cooling bath is removed and the reaction mixture stirred until completion of the reaction. A standard workup and purification using standard protocols to afford 8-bromo-1-methyl-3H-pyrano[3,4,5-cd]indazole-3,5(1H)-dione 8.

Step 7: To a solution of 8-bromo-1-methyl-3H-pyrano[3,4,5-cd]indazole-3,5(1H)-dione 8 (1 eq, 18.05 mmol) and hydroxylamine hydrochloride (1 eq, 1.25 g, 18.05 mmol, 750.92 μL) in pyridine (10 vol eq.) was heated at reflux for 5 h, followed by cooling to 80° C. and the addition of 4-toluenesulfonyl chloride (2 eq). After addition, the temperature was raised and the reaction was stirred at reflux for 5 h, followed by cooling. The reaction mixture was poured into water and extracted with EtOAc (3×). Organics combined, washed with water, sat. aq. NaHCO₃, brine, dried over sodium sulfate, filtered and evaporated to dryness. To a stirred solution of the residue in EtOH (10 vol eq) and water (10 vol eq) is added to 1M aqueous sodium hydroxide (10 eq) dropwise. Thereafter, the mixture was stirred at reflux for 3 h while distilling off the ethanol. After completion of the reaction, the reaction mixture was cooled to 75° C., and hydrochloric acid, 36% w/w aq. soln. (10 vol eq) was added dropwise. Standard work up and purification followed by separation of regioisomers to afford 3-bromo-2-methyl-2,6-dihydro-7H-pyrrolo[4,3,2-cd]indazol-7-one 9 and 3-bromo-2-methyl-2,7-dihydro-6H-pyrrolo[2,3,4-cd]indazol-6-one 10.

Step 8: To a solution of 3-bromo-2-methyl-2,6-dihydro-7H-pyrrolo[4,3,2-cd]indazol-7-one 9 in THE at 0° C. is added NaH (60% dispersion in mineral oil, 10-15 eq) in portions, the cooing bath is removed and the reaction mixture is stirred at this temperature for 1 hours. The reaction mixture is cooled to 0° C., 3-bromo-glutarimide 11 (5-8 eq) is added in portions, the cooling bath removed, and slowly heated to 70° C. until the reaction is judged complete. A standard workup and purification using standard protocols to afford 3-(3-bromo-2-methyl-7-oxo-2,7-dihydro-6H-pyrrolo[4,3,2-cd]indazol-6-yl)piperidine-2,6-dione Compound 12.

Step 9: To a solution of 3-bromo-2-methyl-2,7-dihydro-6H-pyrrolo[2,3,4-cd]indazol-6-one 10 in THE at 0° C. is added NaH (60% dispersion in mineral oil, 10-15 eq) in portions, the cooing bath is removed and the reaction mixture is stirred at this temperature for 1 hours. The reaction mixture is cooled to 0° C., 3-bromo-glutarimide 11 (5-8 eq) is added in portions, the cooling bath removed, and slowly heated to 70° C. until the reaction is judged complete. A standard workup and purification using standard protocols to afford 3-(3-bromo-2-methyl-6-oxo-2,6-dihydro-7H-pyrrolo[2,3,4-cd]indazol-7-yl)piperidine-2,6-dione Compound 13.

### Example 12. Synthesis of 3-(6-bromo-2-oxo-3,4-dihydro-5-oxa-1,2a-diazaacenaphthylen-1(2H)-yl)piperidine-2,6-dione (Compound 14)

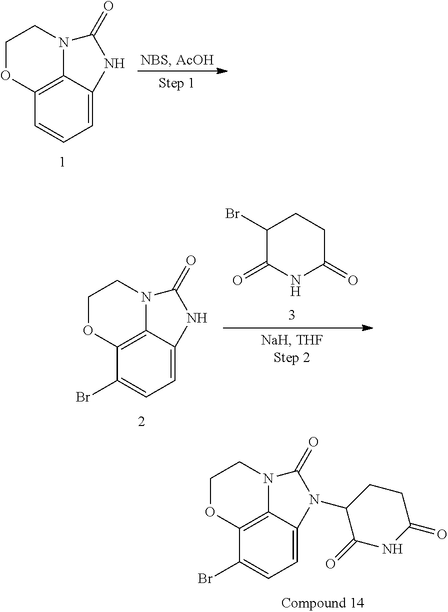

Step 1: To a solution of 3,4-dihydro-5-oxa-1,2a-diazaacenaphthylen-2(1H)-one 1 (1 eq) (CAS #: 1267075-60-4) in acetic acid is added N-Bromosuccinimide (1.2 eq) at room temperature. The reaction mixture is stirred at rt until judged complete. The reaction mixture is then subject to a standard work up and purification to afford 6-bromo-3,4-dihydro-5-oxa-1,2a-diazaacenaphthylen-2(1H)-one 2.

Step 2: To a solution of 6-bromo-3,4-dihydro-5-oxa-1,2a-diazaacenaphthylen-2(1H)-one 2 (1 eq) in THE at 0° C. is added NaH (60% dispersion in mineral oil, 10-15 eq) in portions, the cooing bath is removed and the reaction mixture is stirred at this temperature for 1 hours. The reaction mixture is cooled to 0° C., 3-bromo-glutarimide 3 (5-8 eq) is added in portions, the cooling bath removed, and slowly heated to 70° C. until the reaction is judged complete. Standard workup and purification using standard protocols to afford 3-(6-bromo-2-oxo-3,4-dihydro-5-oxa-1,2a-diazaacenaphthylen-1(2H)-yl)piperidine-2,6-dione Compound 14.

### Example 13. Synthesis of 3-(7-bromo-2-oxo-5,6-dihydro-4H-imidazo[4,5,1-ij]quinolin-1(2H)-yl)piperidine-2,6-dione (Compound 15)

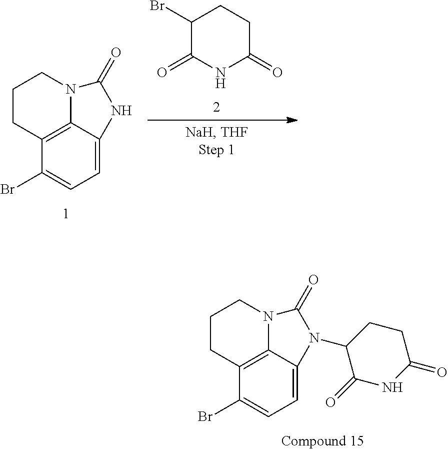 [SMILES: O=c1[nH]c2ccc(Br)c3c2n1CCC3 ; O=C1CCC(Br)C(=O)N1 ; O=C1CCC(n2c(=O)n3c4c(c(Br)ccc42)CCC3)C(=O)N1]

Step 1: To a solution of 7-bromo-5,6-dihydro-4H-imidazo[4,5,1-ij]quinolin-2(1H)-one 1 (1 eq) (CAS #: 1609453-63-5) in THF at 0° C. is added NaH (60% dispersion in mineral oil, 10-15 eq) in portions, the cooing bath is removed and the reaction mixture is stirred at this temperature for 1 hours. The reaction mixture is cooled to 0° C., 3-bromo-glutarimide 2 (5-8 eq) is added in portions, the cooling bath removed, and slowly heated to 70° C. until the reaction is judged complete. Standard workup and purification using standard protocols to afford 3-(7-bromo-2-oxo-5,6-dihydro-4H-imidazo[4,5,1-ij]quinolin-1(2H)-yl)piperidine-2,6-dione Compound 22.

### Example 14. Synthesis of 3-(5-bromo-1-oxo-6,7-dihydroimidazo[4,5,1-hi]indol-2(1H)-yl)piperidine-2,6-dione (Compound 16)

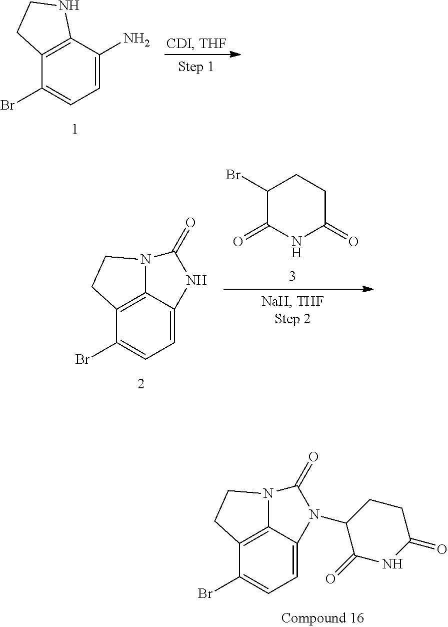

Step 1: To a solution of 4-bromoindolin-7-amine 1 (1 eq) (CAS #: 1783558-27-9) in THE is added 1,1′-carbonyldiimidazole (1.2 eq) at room temperature. The reaction mixture is heated to reflux until judged complete. The cooled reaction mixture is then subject to a standard work up and purification to afford 5-bromo-6,7-dihydroimidazo[4,5,1-hi]indol-1(2H)-one 2.

Step 2: To a solution of 5-bromo-6,7-dihydroimidazo[4,5,1-hi]indol-1(2H)-one 2 (1 eq) in THF at 0° C. is added NaH (60% dispersion in mineral oil, 10-15 eq) in portions, the cooing bath is removed and the reaction mixture is stirred at this temperature for 1 hours. The reaction mixture is cooled to 0° C., 3-bromo-glutarimide 3 (5-8 eq) is added in portions, the cooling bath removed, and slowly heated to 70° C. until the reaction is judged complete. Standard workup and purification using standard protocols to afford 3-(5-bromo-1-oxo-6,7-dihydroimidazo[4,5,1-hi]indol-2(1H)-yl)piperidine-2,6-dione Compound 16.

### Example 15. Synthesis of 3-(7-bromo-2-oxo-4H-imidazo[4,5,1-ij]quinolin-1(2H)-yl)piperidine-2,6-dione (Compound 17)

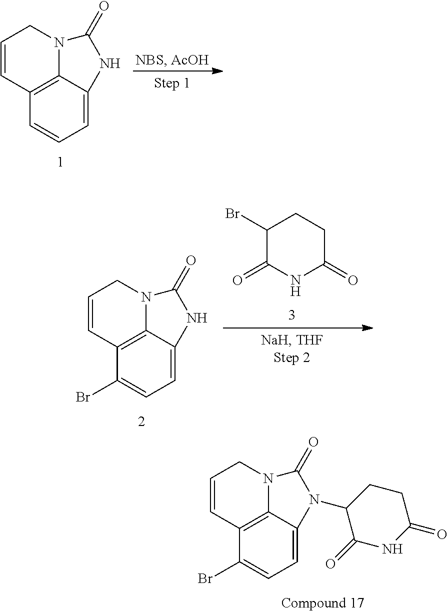

3-(7-bromo-2-oxo-4H-imidazo[4,5,1-ij]quinolin-1(2H)-yl)piperidine-2,6-dione can be prepared in a similar manor to Compound 14, except replacing 3,4-dihydro-5-oxa-1,2a-diazaacenaphthylen-2(1H)-one with 4H-Imidazo[4,5,1-ij]quinolin-2(1H)-one (CAS #83848-83-3).

### Example 16. Synthesis of 3-(5-bromo-1-oxo-2,9a-diazabenzo[cd]azulen-2(1H)-yl)piperidine-2,6-dione (Compound 18)

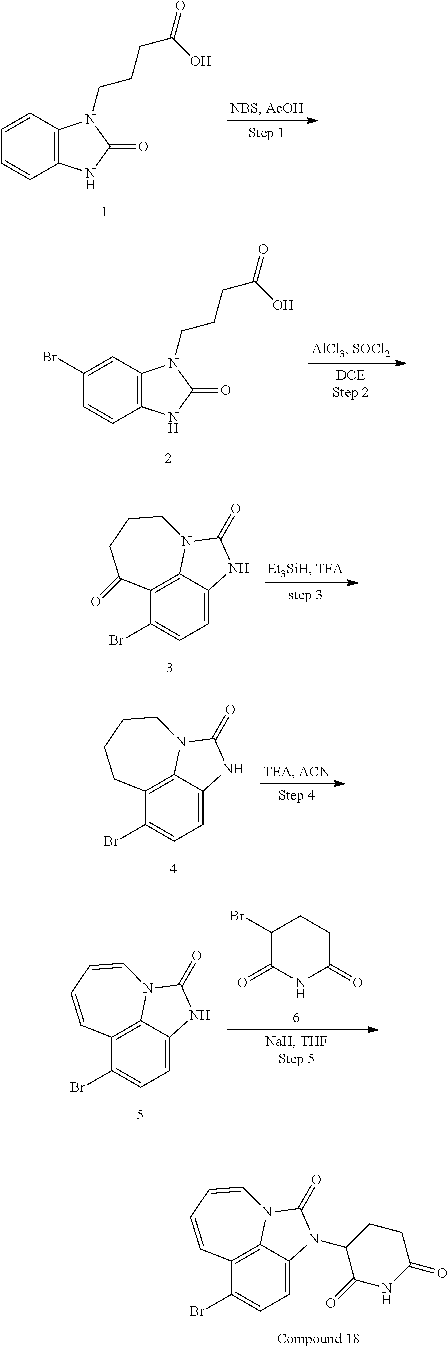

Step 1: To a solution of 4-(2-oxo-2,3-dihydro-1H-benzo[d]imidazol-1-yl)butanoic acid 1 (1 eq) (CAS #: 3273-68-5) in acetic acid is added N-Bromosuccinimide (1.2 eq) at room temperature. The reaction mixture is stirred at RT until judged complete. The reaction mixture is then subject to a standard work up and purification to afford 4-(6-bromo-2-oxo-2,3-dihydro-1H-benzo[d]imidazol-1-yl)butanoic acid 2.

Step 2: To a solution of 4-(6-bromo-2-oxo-2,3-dihydro-1H-benzo[d]imidazol-1-yl)butanoic acid 2 (1 eq) in dichloromethane is added thionyl chloride (2 eq) and the reaction mixture is stirred at room temperature for 2 hours. The mixture is concentrated in vacuo and to the residue is added dichloroethane and aluminum chloride (3 eq), added portion-wise. The reaction mixture is stirred at rt to reflux until judged complete. The reaction mixture is then subject to a standard work up and purification to afford 5-bromo-8,9-dihydro-2,9a-diazabenzo[cd]azulene-1,6(2H,7H)-dione 3.

Step 3: To a solution TFA solution of 5-bromo-8,9-dihydro-2,9a-diazabenzo[cd]azulene-1,6(2H,7H)-dione 3 (1 eq) at 0°, Triethylsilane (1.2 eq) is added slowly and the solution is stirred at 0° until judged complete. The reaction mixture is then subject to a standard work up and purification to afford 5-bromo-6,7,8,9-tetrahydro-2,9a-diazabenzo[cd]azulen-1(2H)-one 4.

Step 4: To a solution of 5-bromo-6,7,8,9-tetrahydro-2,9a-diazabenzo[cd]azulen-1(2H)-one 4 (1 eq) in acetonitrile is added triethylamine (5 eq). The reaction mixture is stirred at RT to reflux until judged complete. The reaction mixture is then subject to a standard work up and purification to afford 5-bromo-2,9a-diazabenzo[cd]azulen-1(2H)-one 5.

Step 5: To a solution of 5-bromo-2,9a-diazabenzo[cd]azulen-1(2H)-one 5 (1 eq) in THF at 0° C. is added NaH (60% dispersion in mineral oil, 10-15 eq) in portions, the cooing bath is removed and the reaction mixture is stirred at this temperature for 1 hour. The reaction mixture is cooled to 0° C., 3-bromo-glutarimide 6 (5-8 eq) is added in portions, the cooling bath removed, and slowly heated to 70° C. until the reaction is judged complete. Standard workup and purification using standard protocols to afford 3-(5-bromo-1-oxo-2,9a-diazabenzo[cd]azulen-2(1H)-yl)piperidine-2,6-dione Compound 18.

### Example 17. Synthesis of 3-(5-bromo-7,8-dihydro-6H-pyrazolo[4,5,1-ij]quinolin-2-yl)piperidine-2,6-dione (Compound 19)

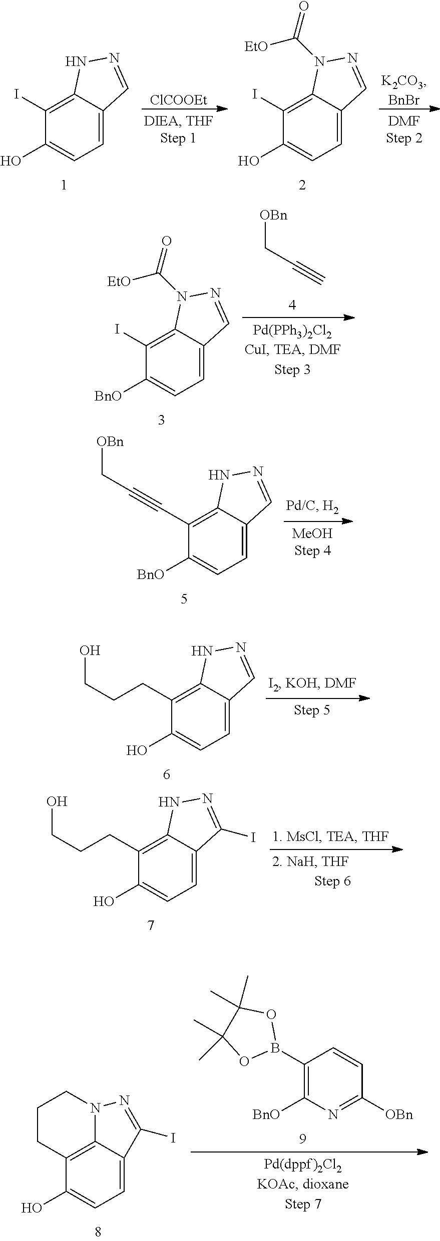

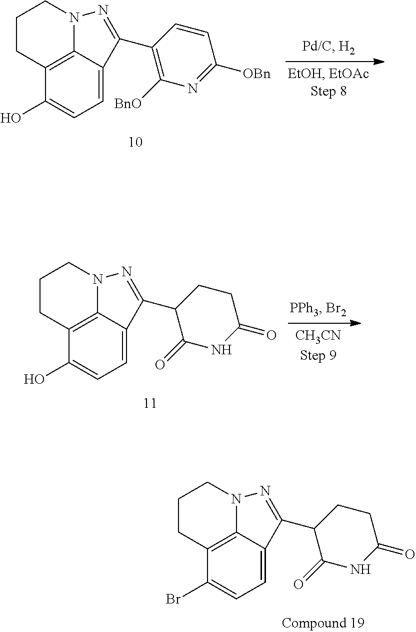 [SMILES: Oc1ccc2c(-c3ccc(OCc4ccccc4)nc3OCc3ccccc3)nn3c2c1CCC3 ; O=C1CCC(c2nn3c4c(c(O)ccc24)CCC3)C(=O)N1 ; O=C1CCC(c2nn3c4c(c(Br)ccc24)CCC3)C(=O)N1]

Step 1: To a solution of 7-iodo-1H-indazol-6-ol 1 (1 eq) (CAS #: 1190314-62-5) in THE is added DIEA (1.2 eq), followed by ethyl chloroformate (1.1 eq) at 0° C. The reaction mixture is stirred at RT until judged complete. The reaction mixture is then subject to a standard work up and purification to afford ethyl 6-hydroxy-7-iodo-1H-indazole-1-carboxylate 2.

Step 2: To a solution of ethyl 6-hydroxy-7-iodo-1H-indazole-1-carboxylate 2 (1 eq) in DMF is added potassium carbonate (1.5 eq), followed by benzyl bromide (1.1 eq) at 0° C. The reaction mixture is stirred at RT until judged complete. The reaction mixture is then subject to a standard work up and purification to afford ethyl 6-(benzyloxy)-7-iodo-1H-indazole-1-carboxylate 3.

Step 3: To a solution of ethyl 6-(benzyloxy)-7-iodo-1H-indazole-1-carboxylate 3 (1 eq) and Benzyl propargyl ether 4 (1.5 eq) (CAS #: 4039-82-1) are dissolved in DMF and TEA (3 eq) is added. The mixture was degassed with Argon. Pd(PPh₃)2Cl2 (0.1 eq) and Copper (I) iodide (0.1 eq) are added and the mixture is sealed and heated at 80° C. in microwave until judged complete. The reaction mixture is then subject to a standard work up and purification to afford 6-(benzyloxy)-7-(3-(benzyloxy)prop-1-yn-1-yl)-1H-indazole 5.

Step 4: To a solution of 6-(benzyloxy)-7-(3-(benzyloxy)prop-1-yn-1-yl)-1H-indazole 5 (1 eq) is added Pd/C (10%, 10 eq) under N₂ atmosphere. The suspension is degassed and purged with H₂ 3 times. The mixture is stirred under H₂ (15 psi) at RT until judged complete. The reaction mixture is then subject to a standard work up and purification to afford 7-(3-hydroxypropyl)-1H-indazol-6-ol 6.

Step 5: To a solution of 7-(3-hydroxypropyl)-1H-indazol-6-ol 6 (1 eq) in DMF is added KOH (3 eq) and I₂ (1.5 eq). The mixture is stirred at RT until judged complete. The reaction mixture is then subject to a standard work up and purification to afford 7-(3-hydroxypropyl)-3-iodo-1H-indazol-6-ol 7.

Step 6: To a solution of 7-(3-hydroxypropyl)-3-iodo-1H-indazol-6-ol 7 (1 eq) in THF is added TEA (2 eq), followed by mesyl chloride (1.2 eq). The mixture is stirred at RT until judged complete. Solvent is removed and the residue is dissolved in THE and cooled to 0° C. NaH (60% in mineral oil, 2.2 eq) is added portion-wise and the mixture is stirred at RT until judged complete. The reaction mixture is then subject to a standard work up and purification to afford 2-iodo-7,8-dihydro-6H-pyrazolo[4,5,1-ij]quinolin-5-ol 8.

Step 7: To a solution of 2,6-bis(benzyloxy)-3-(4,4,5,5-tetramethyl-1,3,2-dioxaborolan-2-yl)pyridine 9 (1 eq), 2-iodo-7,8-dihydro-6H-pyrazolo[4,5,1-ij]quinolin-5-ol 8 (1 eq) and Cs₂CO₃ (3 eq) in dioxane and H₂O (v/v 4:1) is added Pd(dppf)Cl₂ (0.1 eq). The reaction mixture is stirred at 100° C. until judged complete. The reaction mixture is then subject to a standard work up and purification to afford 2-(2,6-bis(benzyloxy)pyridin-3-yl)-7,8-dihydro-6H-pyrazolo[4,5,1-ij]quinolin-5-ol 10.

Step 8: To a solution of 2-(2,6-bis(benzyloxy)pyridin-3-yl)-7,8-dihydro-6H-pyrazolo[4,5,1-ij]quinolin-5-ol 10 (1 eq) in EtOH and EtOAc (v/v 1:1) is added Pd/C (10%, 10 eq) under N² atmosphere. The suspension is degassed and purged with H₂ 3 times. The mixture was stirred under H2 (15 psi) at rt until judged complete. The reaction mixture is then subject to a standard work up and purification to afford 3-(5-hydroxy-7,8-dihydro-6H-pyrazolo[4,5,1-ij]quinolin-2-yl)piperidine-2,6-dione 11.

Step 9: To a solution of 3-(5-hydroxy-7,8-dihydro-6H-pyrazolo[4,5,1-ij]quinolin-2-yl)piperidine-2,6-dione 11 (1 eq) in acetonitrile is added triphenylphosphine (1.3 eq) and bromine (2 eq). The reaction mixture is heated under reflux until judged complete. The reaction mixture is then subject to a standard work up and purification to afford 3-(5-bromo-7,8-dihydro-6H-pyrazolo[4,5,1-ij]quinolin-2-yl)piperidine-2,6-dione Compound 19.

### Example 18. Synthesis of 3-(5-bromo-2-thioxobenzo[cd]indol-1(2H)-yl)piperidine-2,6-dione (Compound 20)

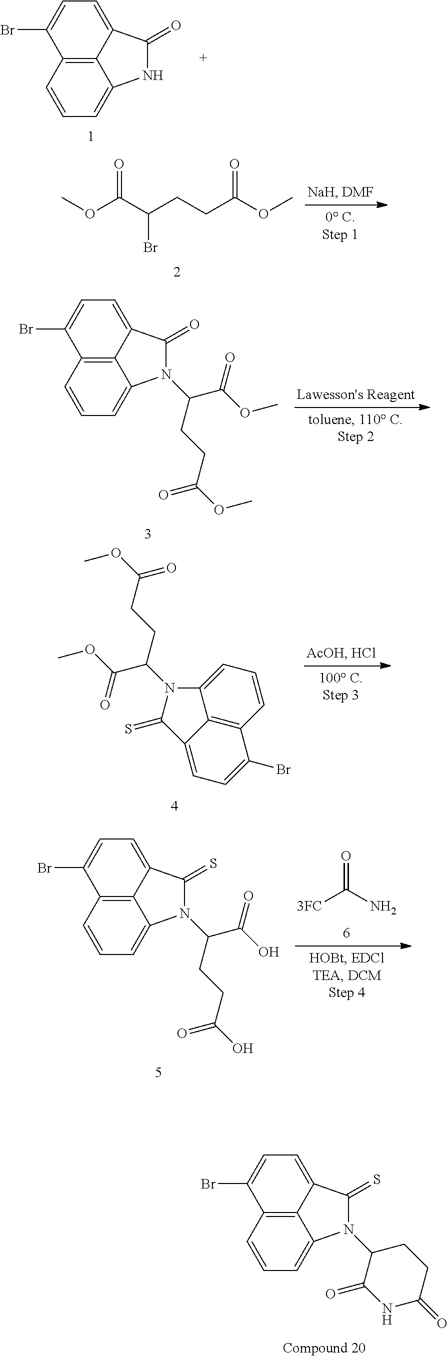

Step 1: To a solution of 5-bromobenzo[cd]indol-2(1H)-one 1 dissolved in anhydrous dimethylformamide add a solution of sodium hydride 60% in mineral oil (1.3 eq). The mixture is stirred at room temperature for 1 hour. To the mixture is added dimethyl 2-bromopentanedioate 2 (CAS: 760-94-1, 1 eq). The resulting mixture is stirred at room temperature for 18 hours. The reaction is subject to standard workup and purification using standard protocols to afford dimethyl 2-(5-bromo-2-oxobenzo[cd]indol-1(2H)-yl)pentanedioate 3. (Similarly described in WO2007056281)

Step 2: To a solution of dimethyl 2-(5-bromo-2-oxobenzo[cd]indol-1(2H)-yl)pentanedioate 3 and Lawesson's reagent (CAS: 19172-47-5, 1 eq) are dissolved in toluene. The resulting mixture is stirred at 110° C. for 10 h. The solvent is evaporated, and purified using standard protocols to afford dimethyl 2-(5-bromo-2-thioxobenzo[cd]indol-1(2H)-yl)pentanedioate 4. (Similarly described in WO 2005/028436 A2)

Step 3: To a solution of dimethyl 2-(5-bromo-2-thioxobenzo[cd]indol-1(2H)-yl)pentanedioate 4, glacial acetic acid and conc. HCl (1:1), and the mixture is stirred at 100° C. for 2.5 h. The reaction is subjected to standard workup, and purified using standard protocols to afford 2-(5-bromo-2-thioxobenzo[cd]indol-1(2H)-yl)pentanedioic acid 5. (Similarly described in WO 2005/028436 A2)

Step 4: To a mixture of 2-(5-bromo-2-thioxobenzo[cd]indol-1(2H)-yl)pentanedioic acid 5, trifluoroacetamide (CAS: 354-38-1, 1.8 eq), HOBt (3.9 eq), EDCI (3.9 eq) and triethylamine (5.5 eq) in CH₂C₂ is stirred at ambient temperature for 3 days. The reaction is subjected to standard work up, and purified using standard protocols to afford 3-(5-bromo-2-thioxobenzo[cd]indol-1(2H)-yl)piperidine-2,6-dione Compound 20. (Similarly described in WO 2005/028436 A2)

### Example 19. Synthesis of 3-(6-bromo-1H-benzo[de]isoquinolin-2(3H)-yl)piperidine-2,6-dione (Compound 21)

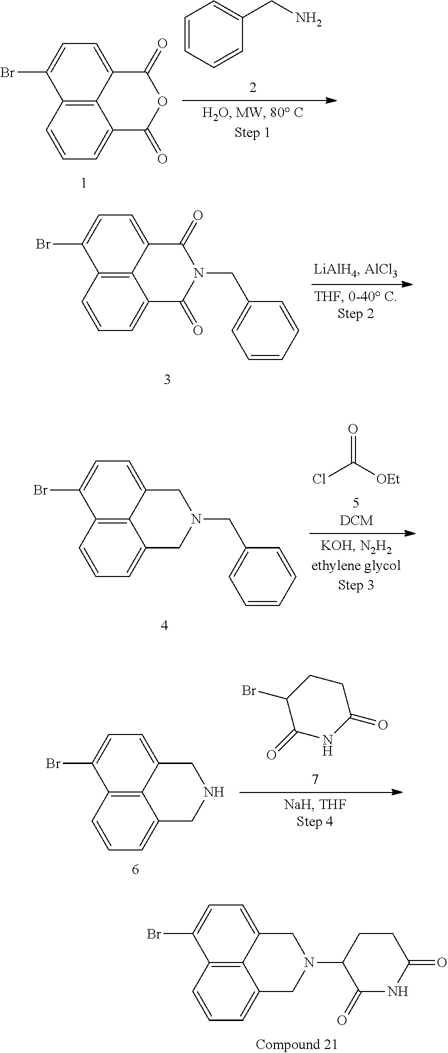

Step 1: To a mixture of benzyl amine 2 (1.2 mmol, CAS: 100-46-9), water (10 mL), and 4-bromo-1,8-naphthalic anhydride 1 (1 mmol, CAS: 81-86-7) was blended together in a sealed and pressurized tube and reacted at 450 W and 80° C. under microwave irradiation for a few minutes. After the reaction, it was filtered to afford 2-benzyl-6-bromo-1H-benzo[de]isoquinoline-1,3(2H)-dione 3 (Yield: 95%). (As described in Synthetic Communications (2012), 42(20), 3042-3052).

Step 2: To a solution of anhydrous aluminum chloride (4.0 mmol) and LiAlH₄ (4.0 mmol) the mixture is added to cold, dry THE (ice bath) with stirring. After removal of the ice bath, 2-benzyl-6-bromo-1H-benzo[de]isoquinoline-1,3(2H)-dione 3 (1.0 mmol) is added in small portions. The mixture is stirred at 40° C. for 5.5 hours and then at RT for 10 hours. The reaction is subjected to standard work up, and purified using standard protocols to afford 2-benzyl-6-bromo-2,3-dihydro-1H-benzo[de]isoquinoline 4. (Similarly described in Journal of the American Chemical Society (2003), 125(19), 5786-5791).

Step 3: To a solution of ethyl chloroformate 5 (21 mmol), a solution of 2-benzyl-6-bromo-2,3-dihydro-1H-benzo[de]isoquinoline 4 (16 mmol) in dry dichloromethane is prepared. The reaction is refluxed for 8 hours. After cooling, the solvent is removed under reduced pressure. A solution of KOH in ethylene glycol (424 mmol) and hydrazine monohydrate (80 mmol) is added to the residue before heating to reflux for 4 hours. After cooling, the reaction is subjected to standard work up, and purified using standard protocols to afford 6-bromo-2,3-dihydro-1H-benzo[de]isoquinoline 6. (Similarly described in Journal of the American Chemical Society (2003), 125(19), 5786-5791).

Step 4: To a solution of 6-bromo-2,3-dihydro-1H-benzo[de]isoquinoline 6 (1 eq) in THE (10 vol eq) at 0° C. is added NaH (5 eq) and stirred at this temp for 15 min before the addition of 3-bromopiperidine-2,6-dione 7 (1 eq). The reaction mixture is slowly heated to 60° C., and stirred at this temperature until completion of the reaction. A standard workup and purified using standard protocols to afford 3-(6-bromo-1H-benzo[de]isoquinolin-2(3H)-yl)piperidine-2,6-dione Compound 21.

### Example 20. Synthesis of 3-(6-bromo-1H-perimidin-1-yl)piperidine-2,6-dione (Compound 22) and 3-(7-bromo-1H-perimidin-1-yl)piperidine-2,6-dione (Compound 23)

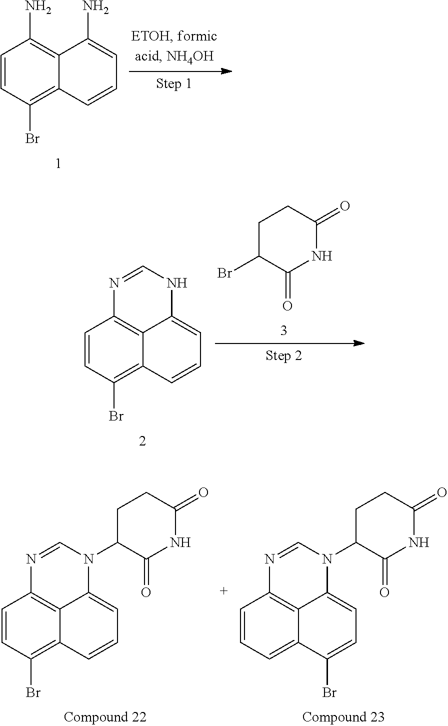 [SMILES: Nc1cccc2c(Br)ccc(N)c12 ; Brc1ccc2c3c(cccc13)NC=N2 ; O=C1CCC(Br)C(=O)N1 ; O=C1CCC(N2C=Nc3ccc(Br)c4cccc2c34)C(=O)N1 ; O=C1CCC(N2C=Nc3cccc4c(Br)ccc2c34)C(=O)N1]

Step 1: To a solution of 4-bromonaphthalene-1,8-diamine 1 (17.1 mmol) is crushed with a mortar and pestle and dissolved in 12 mL of absolute ethanol. Formic acid (106 mmol) is added and the reaction is allowed to stir at reflux for 40 minutes. The reaction is diluted with water (2 mL) and basified with 2N NH₄OH. The resulting precipitate is filtered, washed with ether and recrystallized in ethanol to afford 6-bromo-1H-perimidine 2

Step 2: To a solution of 6-bromo-1H-perimidine 2 (385.65 μmol) is dissolved in THE (10 mL) and then cooled to 0° C. Sodium hydride (in oil dispersion) 60% dispersion in mineral oil (147.77 mg, 3.86 mmol) is added portion wise and stirred at 0° C. for 30 mins. 3-bromopiperidine-2,6-dione 3 (1.93 mmol) is added and reaction mixture is stirred at RT for 30 mins and then stirred at 0° C. for 16 hours. The progress of the reaction is monitored by TLC and after reaction completion reaction mixture is quenched with chilled water, extracted with ethyl acetate, and washed with brine. The organic layer is separated and dried over anhydrous sodium sulfate, filtered and concentrated under reduced pressure to afford the crude compound. The crude material is purified by column chromatography by eluting with 10 to 50% ethyl acetate to afford 3-(6-bromo-1H-perimidin-1-yl)piperidine-2,6-dione Compound 29 and 3-(7-bromo-1H-perimidin-1-yl)piperidine-2,6-dione Compound 30.

### Example 21. Synthesis of 3-(7-bromo-1H-benzo[de]cinnolin-1-yl)piperidine-2,6-dione (Compound 24)

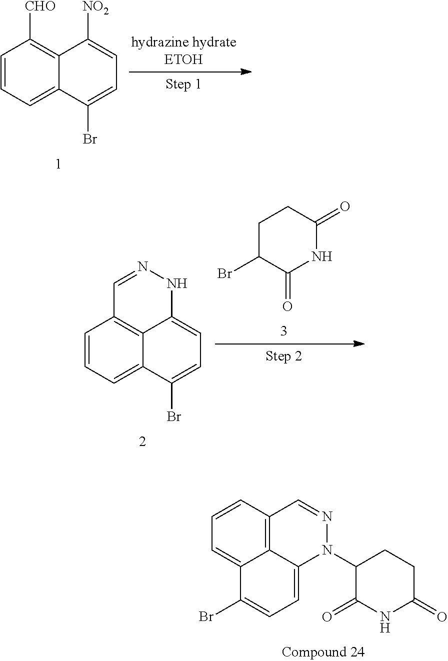 [SMILES: O=Cc1cccc2c(Br)ccc([N+](=O)[O-])c12 ; Brc1ccc2c3c(cccc13)C=NN2 ; O=C1CCC(Br)C(=O)N1 ; O=C1CCC(N2N=Cc3cccc4c(Br)ccc2c34)C(=O)N1]

Step 1: To a mixture of 1 mmol of 5-bromo-8-nitro-1-naphthaldehyde 1 and 1 ml of 88% hydrazine hydrate in 10 ml of ethanol was heated for 6 h under reflux in an argon atmosphere. The mixture was cooled and poured into 20 ml of water and the precipitate was filtered off and dried to afford 7-bromo-1-benzo[de]cinnoline 2.

Step 2: To a solution of 7-bromo-1H-benzo[de]cinnoline 2 (385.65 μmol) is dissolved in THF (10 mL) and then cooled to 0° C. Sodium hydride (in oil dispersion) 60% dispersion in mineral oil (147.77 mg, 3.86 mmol) is added portion wise and stirred at 0° C. for 30 mins. 3-bromopiperidine-2,6-dione (1.93 mmol) is added and reaction mixture is stirred at RT for 30 mins and then stirred at 0° C. for 16 hours. The progress of the reaction is monitored by TLC and after reaction completion reaction mixture is quenched with chilled water, extracted with ethyl acetate, and washed with brine. The organic layer is separated and dried over anhydrous sodium sulfate, filtered and concentrated under reduced pressure to afford the crude compound. The crude material is purified by column chromatography by eluting with 10 to 50% ethyl acetate to afford 3-(7-bromo-1H-benzo[de]cinnolin-1-yl)piperidine-2,6-dione Compound 24.

### Example 22. Synthesis of 3-(6-bromo-1H-naphtho[1,8-de][1,2,3]triazin-1-yl)piperidine-2,6-dione (Compound 25) and 3-(7-bromo-1H-naphtho[1,8-de][1,2,3]triazin-1-yl)piperidine-2,6-dione (Compound 26)

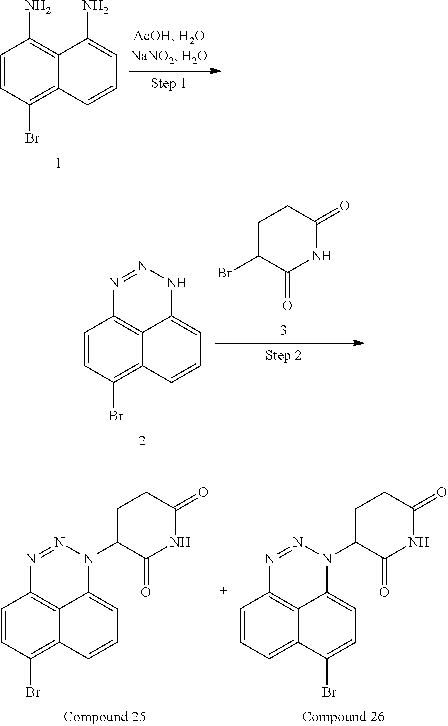 [SMILES: Nc1cccc2c(Br)ccc(N)c12 ; Brc1ccc2c3c(cccc13)NN=N2 ; O=C1CCC(Br)C(=O)N1 ; O=C1CCC(N2N=Nc3ccc(Br)c4cccc2c34)C(=O)N1 ; O=C1CCC(N2N=Nc3cccc4c(Br)ccc2c34)C(=O)N1]

Step 1: To a solution of 4-bromonaphthalene-1,8-diamine 1 (0.014 mol) is suspended in H₂O (600 mL) and AcOH (20 mL) and refluxed. The hot suspension is filtered (filter crucible with celite) and cooled to RT. NaNO₂ (1.55 g, 0.032 mol) in H₂O (20 mL) is added dropwise. The reaction mixture is stirred (5 hours), filtered (filter crucible), washed with hot H₂O and dried overnight to afford 6-bromo-1H-naphtho[1,8-de][1,2,3]triazine 2.

Step 2: To a solution of 6-bromo-1H-naphtho[1,8-de][1,2,3]tri azine 2 (385.65 μmol) is dissolved in THE (10 mL) and then cooled to 0° C. Sodium hydride (in oil dispersion) 60% dispersion in mineral oil (147.77 mg, 3.86 mmol) is added portion wise and stirred at 0° C. for 30 mins. 3-bromopiperidine-2,6-dione 3 (1.93 mmol) is added and reaction mixture is stirred at RT for 30 mins and then stirred at 0° C. for 16 hours. The progress of the reaction is monitored by TLC and after reaction completion reaction mixture is quenched with chilled water, extracted with ethyl acetate, and washed with brine. The organic layer is separated and dried over anhydrous sodium sulfate, filtered and concentrated under reduced pressure to afford the crude compound. The crude material is purified by column chromatography by eluting with 10 to 50% ethyl acetate to afford 3-(6-bromo-1H-naphtho[1,8-de][1,2,3]triazin-1-yl)piperidine-2,6-dione Compound 32 and 3-(7-bromo-1H-naphtho[1,8-de][1,2,3]triazin-1-yl)piperidine-2,6-dione Compound 33.

### Example 23. Synthesis of 3-(6-bromo-2H-naphtho[1,8-cd]isoxazol-2-yl)piperidine-2,6-dione (Compound 27)

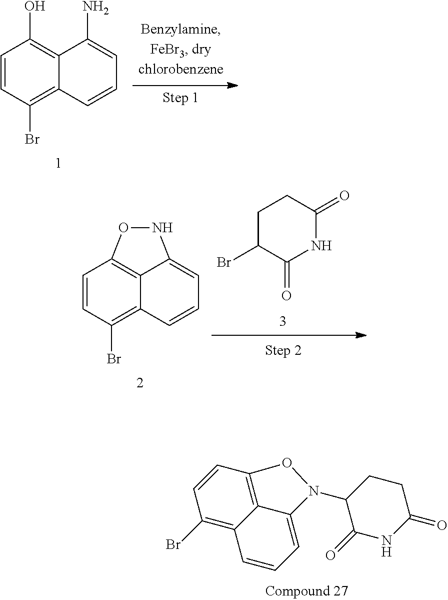

Step 1: To a solution of 8-amino-4-bromonapthalenol 1 (1.0 mmol), benzylamine (1.3 mmol), FeBr₃ and dry chlorobenzene (1 mL) are added to an oven-dried Schlenk tube. The tube is equipped with a molecular oxygen balloon. The reaction mixture is stirred constantly at 110° C. The reaction is monitored to complete consumption of starting material by TLC. The reaction is cooled to RT. The reaction is diluted with CH₂C₂ and washed with water. The organic layer is dried over anhydrous sodium sulfate, filtered and concentrated under reduced pressure. The crude material is purified by silica column chromatography (ethyl acetate/hexanes) to afford 6-bromo-2H-naptho[1,8-cd]isoxazole 2.

Step 2: To a stirred solution of 3-bromopiperidine-2,6-dione 3 (1.0 mmol) and DIPEA (2.5 mmol) in DMF (3 mL) was added 6-bromo-2H-naptho[1,8-cd]isoxazole 2 (2.5 mmol). The resulting solution was heated at 80° C.-100° C. for 5-hours. The reaction mixture is then cooled to room temperature and evaporated under reduced pressure. The crude reaction mass is purified by reverse phase preparative HPLC to afford 3-(6-bromo-2H-naphtho[1,8-cd]isoxazol-2-yl)piperidine-2,6-dione Compound 27.

### Example 24. Synthesis of 3-(6-bromo-2-oxo-2,3-dihydro-1H-perimidin-1-yl)piperidine-2,6-dione (Compound 28) and 3-(7-bromo-2-oxo-2,3-dihydro-1H-perimidin-1-yl)piperidine-2,6-dione (Compound 29)

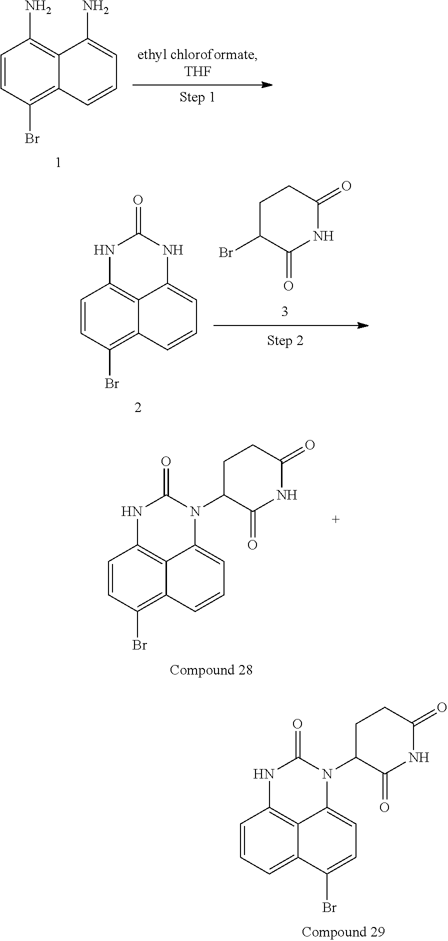 [SMILES: Nc1cccc2c(Br)ccc(N)c12 ; O=C1Nc2cccc3c(Br)ccc(c23)N1 ; O=C1CCC(Br)C(=O)N1 ; O=C1CCC(N2C(=O)Nc3ccc(Br)c4cccc2c34)C(=O)N1 ; O=C1CCC(N2C(=O)Nc3cccc4c(Br)ccc2c34)C(=O)N1]

Step 1: To a solution of 4-bromonaphthalene-1,8-diamine 1 (31.6 mmol) in 100 mL THF was added dropwise a solution of ethyl chloroformate (31.6 mmol) in 10 mL THF over a period of 30 min at 0° C. The mixture is stirred at 25° C. for 1 day and then was heated at 40° C. for 2 hours. The precipitate is filtered and washed with CH₂Cl₂ to afford 6-bromo-1H-perimidin-2(3H)-one.

Step 2: To a solution of 6-bromo-1H-perimidin-2(3H)-one 2 (385.65 μmol) is dissolved in THF (10 mL) and then cooled to 0° C. Sodium hydride (in oil dispersion) 60% dispersion in mineral oil (147.77 mg, 3.86 mmol) is added portion wise and stirred at 0° C. for 30 mins. 3-bromopiperidine-2,6-dione 3 (1.93 mmol) is added and reaction mixture is stirred at RT for 30 mins and then stirred at 0° C. for 16 hours. The progress of the reaction is monitored by TLC and after reaction completion reaction mixture is quenched with chilled water, extracted with ethyl acetate, and washed with brine. The organic layer is separated and dried over anhydrous sodium sulfate, filtered and concentrated under reduced pressure to afford the crude compound. The crude material is purified by column chromatography by eluting with 10 to 50% ethyl acetate to afford 3-(6-bromo-2-oxo-2,3-dihydro-1H-perimidin-1-yl)piperidine-2,6-dione Compound 35 and 3-(7-bromo-2-oxo-2,3-dihydro-1H-perimidin-1-yl)piperidine-2,6-dione Compound 36.

### Example 25. Synthesis of 3-(6-bromo-2-oxo-2,3-dihydro-1H-benzo[de]quinolin-1-yl)piperidine-2,6-dione (Compound 30)

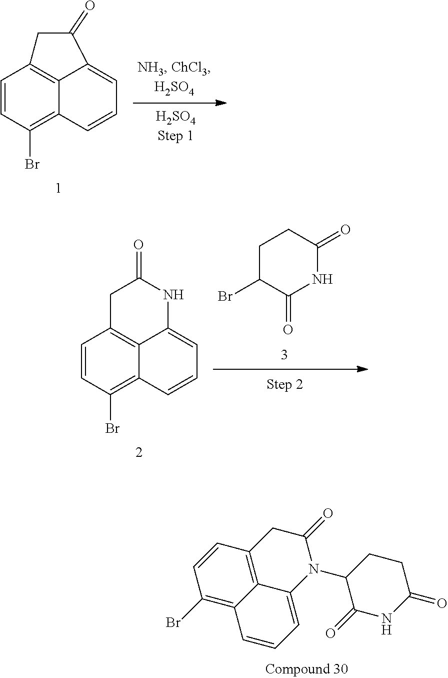 [SMILES: O=C1Cc2ccc(Br)c3cccc1c23 ; O=C1Cc2ccc(Br)c3cccc(c23)N1 ; O=C1CCC(Br)C(=O)N1 ; O=C1CCC(N2C(=O)Cc3ccc(Br)c4cccc2c34)C(=O)N1]

Step 1: To a mixture of 5˜bromoacenaphthylene-1(21)-one 1 (3 g) and 0.8N NH₃ in 80 ml CHCl₃, is stirred with 2 ml concentrated H₂SO₄ at 50° C. for 0.5 hour and then cooled to 0° C. The mixture is neutralized with satd. aq. KHCO₃, and filtered. The organic layer of the filtrate is worked up, and the resulting residue is purified by silica gel chromatography (ethyl acetate/hexaness) to afford 6-bromo-H-benzo[de]quinolin-2(3H)-one 2.

Step 2: To a solution of 6-bromo-1H-benzo[de]quinolin-2(3H)-one 2 (385.65 μmol) is dissolved in THE (10 mL) and then cooled to 0° C. Sodium hydride (in oil dispersion) 60% dispersion in mineral oil (147.77 mg, 3.86 mmol) is added portion wise and stirred at 0° C. for 30 mins. 3-bromopiperidine-2,6-dione (1.93 mmol) is added and reaction mixture is stirred at RT for 30 mins and then stirred at 0° C. for 16 hours. The progress of the reaction is monitored by TLC and after reaction completion reaction mixture is quenched with chilled water, extracted with ethyl acetate, and washed with brine. The organic layer is separated and dried over anhydrous sodium sulfate, filtered and concentrated under reduced pressure to afford the crude compound. The crude material is purified by column chromatography by eluting with 10 to 50% ethyl acetate to afford 3-(6-bromo-2-oxo-2,3-dihydro-1H-benzo[de]quinolin-1-yl)piperidine-2,6-dione Compound 30.

### Example 26. Synthesis of 3-(6-bromo-2-oxonaphtho[1,8-de][1,3]oxazin-3(2H)-yl)piperidine-2,6-dione (Compound 31) and 3-(7-bromo-2-oxonaphtho[1,8-de][1,3]oxazin-3(2H)-yl)piperidine-2,6-dione (Compound 32)

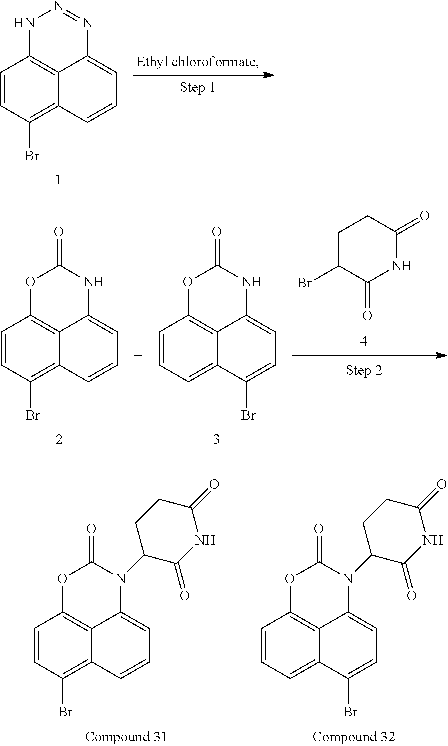

Step 1: To a microwave tube is charged with 6-bromo-1H-naphtho[1,8-de][1,2,3]triazine 1 and ethyl chloroformate, sealed and heated to 200° C. for 4 minutes. The reaction is cooled and concentrated. The crude residue is purified by silica gel chromatography to afford a mixture of 7-bromonaphtho[1,8-de][1,3]oxazin-2(3H)-one 2 and 6-bromonaphtho[1,8-de][1,3]oxazin-2(3H)-one 3.

Step 2: To a mixture of 7-bromonaphtho[1,8-de][1,3]oxazin-2(3H)-one 2 and 6-bromonaphtho[1,8-de][1,3]oxazin-2(3H)-one 3 (385.65 μmol) is dissolved in THF (10 mL) and then cooled to 0° C. Sodium hydride (in oil dispersion) 60% dispersion in mineral oil (147.77 mg, 3.86 mmol) is added portion wise and stirred at 0° C. for 30 mins. 3-bromopiperidine-2,6-dione (1.93 mmol) is added and reaction mixture is stirred at RT for 30 mins and then stirred at 0° C. for 16 hours. The progress of the reaction is monitored by TLC and after reaction completion reaction mixture is quenched with chilled water, extracted with ethyl acetate, and washed with brine. The organic layer is separated and dried over anhydrous sodium sulfate, filtered and concentrated under reduced pressure to afford the crude compound. The crude material is purified by column chromatography by eluting with 10 to 50% ethyl acetate to afford 3-(7-bromo-2-oxonaphtho[1,8-de][1,3]oxazin-3(2H)-yl)piperidine-2,6-dione Compound 31 and 3-(6-bromo-2-oxonaphtho[1,8-de][1,3]oxazin-3(2H)-yl)piperidine-2,6-dione Compound 32.

### Example 27. Synthesis of 3-(6-bromo-1,1-dioxido-2H-naphtho[1,8-cd]isothiazol-2-yl)piperidine-2,6-dione (Compound 33)

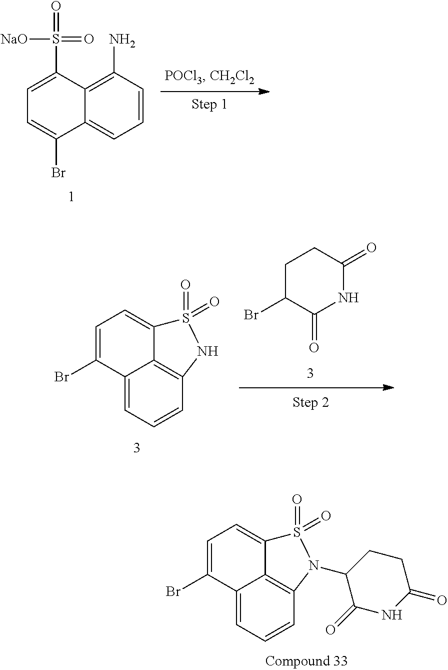

Step 1: To a solution of sodium 4-bromo-8-amino-naphthalene-1-sulfonate 1 (1.2 g, 3.70 mmol) is suspended in phosphorous oxychloride (10 mL, 107 5 mmol) and the mixture is refluxed for 1 hour to give a thin suspension. The mixture is cooled to room temperature and is added to ice (100 mL). The precipitate is collected and washed with water (20 mL) then dried under vacuum. The solid is dissolved in 5% methanol in methylene chloride and placed on a silica gel column and eluted with 5% methanol in methylene chloride to afford 6-bromo-2H-naphtho[1,8-cd]isothiazole 1,1-dioxide 2.

Step 2: To a solution of 6-bromo-2H-naphtho[1,8-cd]isothiazole 1,1-dioxide 2 (385.65 μmol) is dissolved in THE (10 mL) and then cooled to 0° C. Sodium hydride (in oil dispersion) 60% dispersion in mineral oil (147.77 mg, 3.86 mmol) is added portion wise and stirred at 0° C. for 30 mins. 3-bromopiperidine-2,6-dione (1.93 mmol) is added and reaction mixture is stirred at RT for 30 mins and then stirred at 0° C. for 16 hours. The progress of the reaction is monitored by TLC and after reaction completion reaction mixture is quenched with chilled water, extracted with ethyl acetate, and washed with brine. The organic layer is separated and dried over anhydrous sodium sulfate, filtered and concentrated under reduced pressure to afford the crude compound. The crude material is purified by column chromatography by eluting with 10 to 50% ethyl acetate to afford 3-(6-bromo-2-oxo-2,3-dihydro-1H-benzo[de]quinolin-1-yl)piperidine-2,6-dione Compound 33.

### Example 28. Synthesis of 5-(6-bromo-2-oxobenzo[cd]indol-1(2H)-yl)-1,3-oxazinane-2,4-dione (Compound 34)

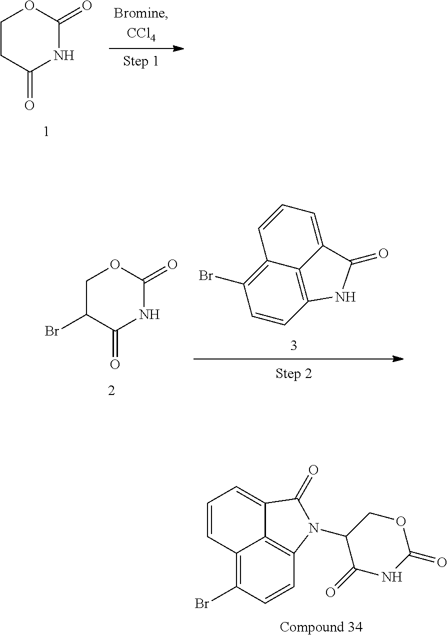

Step 1: To a solution of bromine (87.8 mmol) is added to a suspension of 1,3-oxazinane-2,4-dione 1 (50.3 mmol) suspended in chloroform (20 ml) and the mixture is stirred in a closed vessel for 90 minutes at a bath temperature of 110° C. After cooling, the vessel is opened and stirring is continued until no more hydrogen bromide escapes. The reaction mixture is evaporated in vacuo. The residue is dissolved in ethanol and evaporated to afford 5-bromo-1,3-oxazinane-2,4-dione.

Step 2: To a solution of 6-bromobenzo[cd]indol-2(1H)-one 3 (385.65 μmol) is dissolved in THE (10 mL) and then cooled to 0° C. Sodium hydride (in oil dispersion) 60% dispersion in mineral oil (147.77 mg, 3.86 mmol) is added portion wise and stirred at 0° C. for 30 mins. 5-bromo-1,3-oxazinane-2,4-dione 2 (1.93 mmol) is added and reaction mixture is stirred at RT for 30 mins and then stirred at 0° C. for 16 hours. The progress of the reaction is monitored by TLC and after reaction completion reaction mixture is quenched with chilled water, extracted with ethyl acetate, and washed with brine. The organic layer is separated and dried over anhydrous sodium sulfate, filtered and concentrated under reduced pressure to afford the crude compound. The crude material is purified by column chromatography by eluting with 10 to 50% ethyl acetate to afford 5-(6-bromo-2-oxobenzo[cd]indol-1(2H)-yl)-1,3-oxazinane-2,4-dione Compound 34.

### Example 29. Synthesis of 5-(6-bromo-2-oxobenzo[cd]indol-1(2H)-yl)pyrimidine-2,4(3H,5H)-dione (Compound 35)

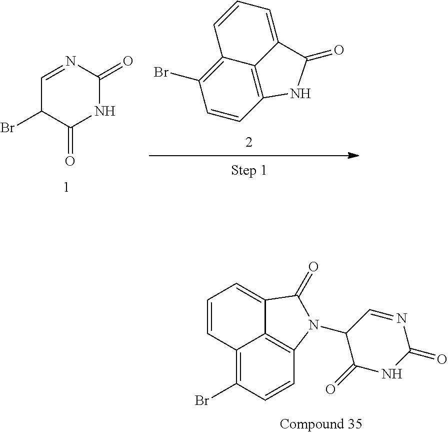

Step 1: To a solution of 6-bromobenzo[cd]indol-2(1H)-one 2 (385.65 μmol) is dissolved in THE (10 mL) and then cooled to 0° C. Sodium hydride (in oil dispersion) 60% dispersion in mineral oil (147.77 mg, 3.86 mmol) is added portion wise and stirred at 0° C. for 30 mins. 5-bromopyrimidine-2,4(3H,5H)-dione 1 (as prepared in PCT Int. Appl., 2016044770, 1.93 mmol) is added and reaction mixture is stirred at RT for 30 mins and then stirred at 0° C. for 16 hours. The progress of the reaction is monitored by TLC and after reaction completion reaction mixture is quenched with chilled water, extracted with ethyl acetate, and washed with brine. The organic layer is separated and dried over anhydrous sodium sulfate, filtered and concentrated under reduced pressure to afford the crude compound. The crude material is purified by column chromatography by eluting with 10 to 50% ethyl acetate to afford 5-(6-bromo-2-oxobenzo[cd]indol-1(2H)-yl)pyrimidine-2,4(3H,5H)-dione Compound 35.

### Example 30. Synthesis of 3-(5-bromo-2-oxo-benzo[cd]indol-1-yl)piperidine-2,6-dione (Compound 36)

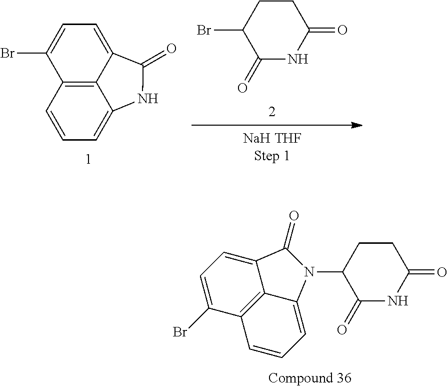

Step 1: To a 500 mL three-necked round bottom flask containing a well stirred solution of 5-bromo-1H-benzo[cd]indol-2-one 1 (2.0 g, 6.85 mmol) in dry THE (200 mL) was added Sodium hydride 60% dispersion in mineral oil (2.63 g, 68.53 mmol) at 0° C. and the reaction mixture was stirred at ambient temperature. After 1 h, 3-bromopiperidine-2,6-dione 2 (6.58 g, 30.84 mmol), dissolved in dry THE (30 mL), was added at 0° C. The reaction mixture was stirred at 65° C. for 16 hours. The reaction mixture was quenched with saturated ammonium chloride solution (50 mL) at 0° C. then extracted with ethyl acetate (2×50 mL). Organic layer collected dried over sodium sulphate, concentrated under reduced pressure to get a crude compound which was purified by flash column chromatography (100 g silica gel column, mobile phase A: Petroleum ether and mobile phase B: ethyl acetate) and compound was eluted at 80-100% ethyl acetate in petroleum ether to afford 3-(5-bromo-2-oxo-benzo[cd]indol-1-yl)piperidine-2,6-dione Compound 35 (1.3 g, 2.85 mmol, 41.57% yield) as a yellow solid. LCMS (ES+): m/z 359.0 [M+H]⁺

### Example 31. Synthesis of 3-(6-bromo-2-oxopyrrolo[4,3,2-ij]isoquinolin-1(2H)-yl)piperidine-2,6-dione (Compound 37)

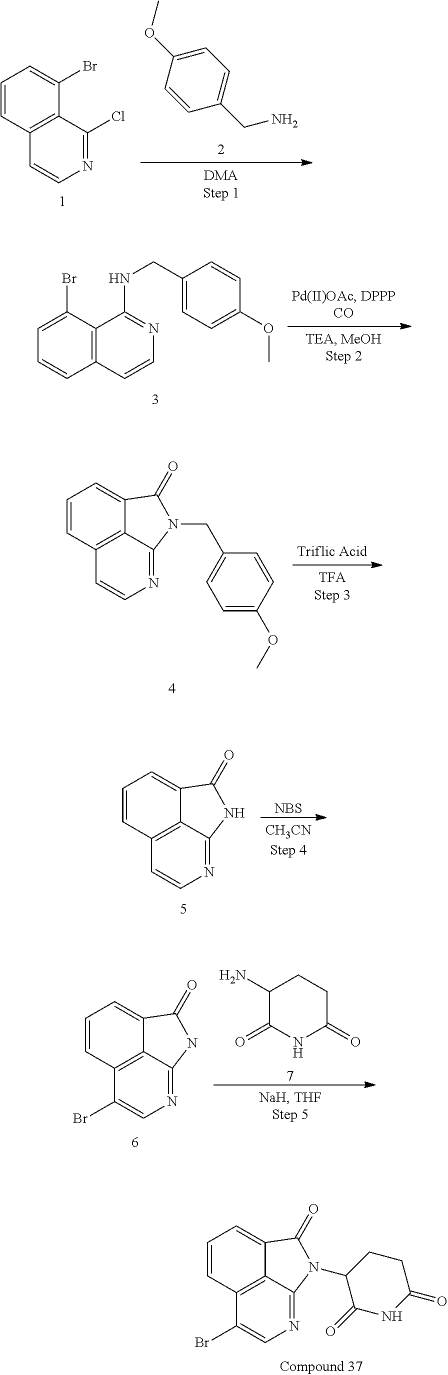

Step 1: To a solution of 8-bromo-1-chloro-isoquinoline 1 (50 g, 206.19 mmol) and 4-methoxy benzylamine 2 (42.43 g, 309.28 mmol, 40.41 mL) in DMA (300 mL) in a sealed vessel was heated at 120° C. for 3 hours. The reaction mixture was diluted with ethyl acetate and water. The organic layer was dried over sodium sulphate and concentrated. The reaction mixture was purified by silica gel column chromatography (5% ethyl acetate in hexanes) to afford 8-bromo-N-(4-methoxybenzyl)isoquinolin-1-amine 3 (52 g, 72%).

Step 2: To a solution of 8-bromo-N-[(4-methoxyphenyl)methyl]isoquinolin-1-amine 3 (52 g, 151.51 mmol) in MeOH (500 mL) was added triethyl amine (61.32 g, 606.03 mmol, 84.47 mL) then purged with argon for 10 minutes. DPPP (12.50 g, 30.30 mmol) and Palladium (II) acetate (3.40 g, 15.15 mmol) was added and the reaction mixture was shaken in a Parr-autoclave at 100° C. under an atmosphere of 70 Psi of carbon monoxide. The reaction mixture was filtered through a celite bed and concentrated. The crude material was worked up with ethyl acetate and water followed by brine. The organic layer was dried over sodium sulphate and concentrated. the crude material was purified by silica gel column chromatography (60% ethyl acetate in hexanes) to afford the 19-[(4-methoxyphenyl)methyl]-18,19-diazatricyclododeca-1(3),2(12),8,14,16(18)-pentaen-17-one 4 (44 g, 90%) as off white solid.

Step 3: To a cooled solution of 19-[(4-methoxyphenyl)methyl]-18,19-diazatricyclododeca-1(3),2(12),8,14,16(18)-pentaen-17-one 4 (1 g, 3.44 mmol) in TFA (12 mL) was added Triflic acid (3.62 g, 24.11 mmol, 2.12 mL) was added dropwise. The cooling bath was removed and the reaction mixture was stirred at 25° C. for 14 hours. The reaction mixture was evaporated and quenched with saturated sodium bicarbonate solution, extracted with ethyl acetate, washed with water followed by brine. The organic part was dried over sodium sulphate and concentrated to give 10,11-diazatricyclododeca-(2),1(5),3,6,8(10)-pentaen-9-one 5 (580 mg 82%).

Step 4: To a stirred suspension of 10,11-diazatricyclododeca-(2),1(5),3,6,8(10)-pentaen-9-one 5 (85 mg, 499.51 μmol) in acetonitrile (3 mL) at 0° C. was added N-bromosuccinimide (88.90 mg, 499.51 μmol, 42.33 μL), the cooling bath removed and the reaction mixture was stirred at 25° C. for 14 hours. The reaction mixture was evaporated, quenched with saturated Na₂S₂O₃ solution extracted with ethyl acetate. The organic layer washed with water followed by brine and dried over sodium sulphate, concentrated, purified by silica gel column chromatography (60% ethyl acetate in hexanes) to afford 6-bromo-10,11-diazatricyclododeca-(2),1(4),3(6),5(7),8(10)-pentaen-9-one 6 as yellowish solid (40 mg, 31%).

Step 5: To a solution of 6-bromo-10,11-diazatricyclododeca-(2),1(4),3(6),5(7),8(10)-pentaen-9-one 6 (1 eq) in THE (10 vol eq) at 0° C. is added NaH (5 eq) and stirred at this temp for 15 min before the addition of 3-bromopiperidine-2,6-dione 7 (1 eq). The reaction mixture is slowly heated to 60° C., and stirred at this temperature until completion of the reaction. A standard workup and purification using standard protocols to afford 3-(6-bromo-2-oxopyrrolo[4,3,2-ij]isoquinolin-1(2H)-yl)piperidine-2,6-dione Compound 37.

### Example 32. Synthesis of 3-(6-bromo-2-oxopyrrolo[2,3,4-de]isoquinolin-1(2H)-yl)piperidine-2,6-dione (Compound 38)

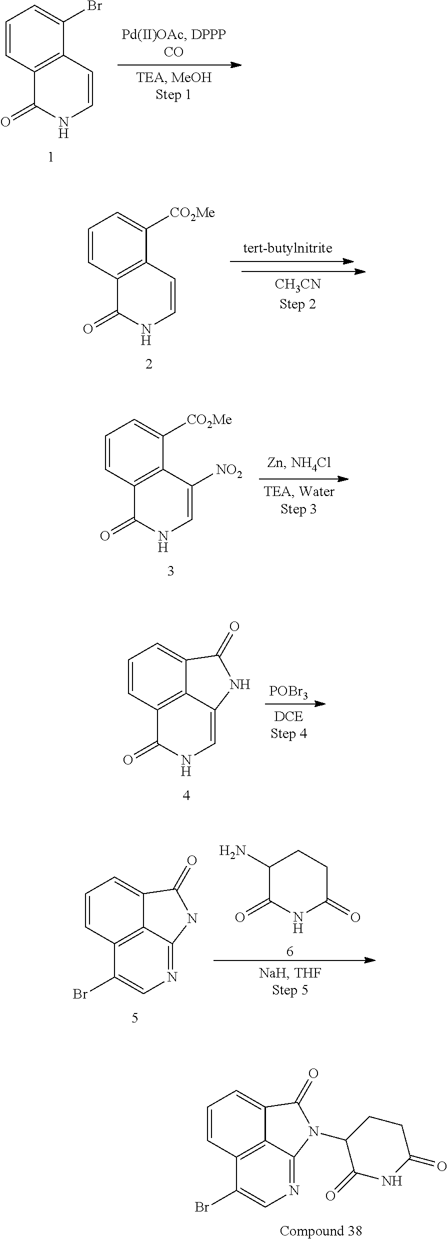

Step 1: To a stirred solution of 5-bromo-2H-isoquinolin-1-one 1 (18 g, 80.34 mmol) and 1,3-bis(diphenylphosphino)propane (6.63 g, 16.07 mmol) in methanol (50.0 mL) was degassed with argon for 5 minutes, followed by addition of triethylamine, 99% (32.52 g, 321.35 mmol, 44.79 mL) and diacetoxypalladium (1.80 g, 8.03 mmol) into the reaction mixture. Resultant reaction mixture was heated in PAR AUTOCLAVE in 80 psi of CO₂ at 100° C. for 12 hours. The reaction mixture was filtered through cellite, filtrate was concentrated and purification by silica gel column chromatography (40% ethyl acetate in Hexanes) to afford methyl 1-oxo-2H-isoquinoline-5-carboxylate 2 (12 g, 54.92 mmol, 68.36% yield) as grey colored solid.

Step 2: To a stirred solution of methyl 1-oxo-2H-isoquinoline-5-carboxylate 2 (7.7 g, 37.89 mmol) in acetonitrile (100 mL) was added tert-butylnitrite (15.63 g, 151.58 mmol, 18.03 mL). The reaction mixture was heated at 60° C. for 16 hours, then concentrated under reduced pressure. The crude material was treated with acetonitrile (20 ml), cooled to 0° C., stirred for 20 minutes and filtered. Solid residue was washed with ether and was dried under reduced pressure to afford methyl 4-nitro-1-oxo-2H-isoquinoline-5-carboxylate 3 (4.5 g, 17.42 mmol, 45.97% yield) as white solid.

Step 3: To a stirred solution of methyl 4-nitro-1-oxo-2H-isoquinoline-5-carboxylate 3 (2 g, 8.06 mmol) in THE (20 mL) and water (5 mL), zinc (526.93 mg, 8.06 mmol, 73.80 μL), and ammonium chloride (431.05 mg, 8.06 mmol, 281.73 μL) were added at room temperature. The RM was then heated at 70° C. for 12 hours. The cooled RM was filtered through celite and the filtrate and concentrated to afford crude 10,11-diazatricyclododeca-(2),1(4),3(6),5(7)-tetraene-8,9-dione 4 (800 mg, 3.44 mmol, 42.66% yield) as yellow solid. Used in the next step without further purification.

Step 4: To a stirred solution of 10,11-diazatricyclododeca-(2),1(4),3(6),5(7)-tetraene-8,9-dione 4 (500 mg, 2.69 mmol) in DCE (40 mL) was added phosphoryl bromide (615.99 mg, 2.15 mmol, 218.43 μL) and the reaction mixture heated at 90° C. for 16 hours. The reaction mixture was cooled to RT, poured to ice water, basified with sodium bicarbonate, extracted with ethyl acetate, washed with brine, dried over sodium sulfate and concentrated under reduced pressure. Crude was purified by combiflash eluting with 20% ethyl acetate in hexanes to afford 8-bromo-10,11-diazatricyclododeca-(2),1(4),3(6),5(7),8(10)-pentaen-9-one 5 (70 mg, 252.95 μmol, 9.42% yield) as yellow solid.

Step 5: To a solution of 8-bromo-10,11-diazatricyclododeca-(2),1(4),3(6),5(7),8(10)-pentaen-9-one 5 (1 eq) in THE (10 vol eq) at 0° C. is added NaH (5 eq) and stirred at this temp for 15 min before the addition of 3-bromopiperidine-2,6-di one 6 (1 eq). The reaction mixture is slowly heated to 60° C. and stirred at this temperature until completion of the reaction. A standard workup and purification using standard protocols to afford 3-(6-bromo-2-oxopyrrolo[2,3,4-de]isoquinolin-1(2H)-yl)piperidine-2,6-di one Compound 38.

### Example 33. The following compounds can be synthesized in a similar manner

| | | Compound |
|---|---|---|
| Intermediate | Product | No. |
| 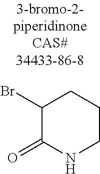 | 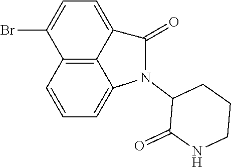 [SMILES: O=C1NCCCC1N1C(=O)c2ccc(Br)c3cccc1c23] | Compound 39 |
| 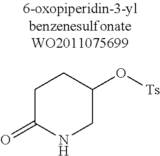 [SMILES: Cc1ccc(S(=O)(=O)OC2CCC(=O)NC2)cc1] | 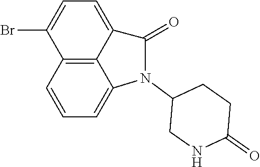 [SMILES: O=C1CCC(N2C(=O)c3ccc(Br)c4cccc2c34)CN1] | Compound 40 |
| 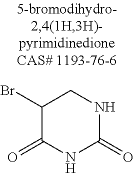 | 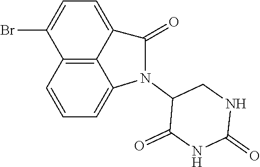 [SMILES: O=C1NCC(N2C(=O)c3ccc(Br)c4cccc2c34)C(=O)N1] | Compound 41 |
| 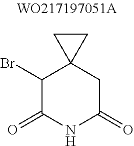 [SMILES: O=C1CC2(CC2)C(Br)C(=O)N1] | 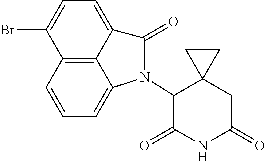 [SMILES: O=C1CC2(CC2)C(N2C(=O)c3ccc(Br)c4cccc2c34)C(=O)N1] | Compound 42 |
| 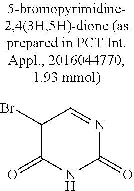 | 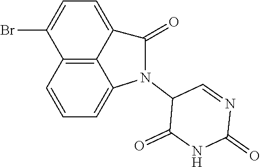 [SMILES: O=C1N=CC(N2C(=O)c3ccc(Br)c4cccc2c34)C(=O)N1] | Compound 43 |

### Example 34. The following amines intermediates can be converted to bromo intermediates using standard chemistry and utilized in the preceding alkylation reaction to prepares the products

| | | | Compound |
|---|---|---|---|
| Amine Starting Material | Bromo intermediate | Product | No. |
| 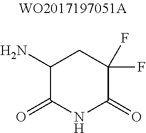 [SMILES: NC1CC(F)(F)C(=O)NC1=O] | 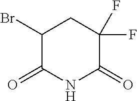 [SMILES: O=C1NC(=O)C(F)(F)CC1Br] | 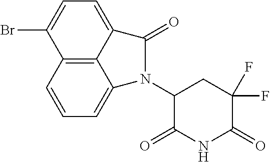 [SMILES: O=C1NC(=O)C(F)(F)CC1N1C(=O)c2ccc(Br)c3cccc1c23] | Compound 44 |
| 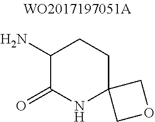 [SMILES: NC1CCC2(COC2)NC1=O] | 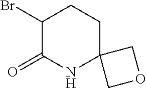 [SMILES: O=C1NC2(CCC1Br)COC2] | 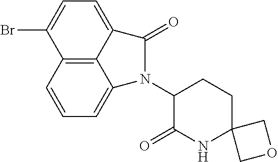 [SMILES: O=C1NC2(CCC1N1C(=O)c3ccc(Br)c4cccc1c34)COC2] | Compound 45 |
| 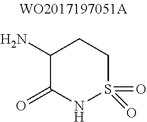 | 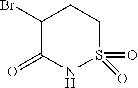 | 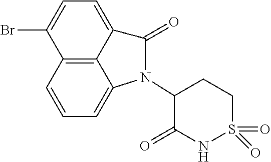 [SMILES: O=C1NS(=O)(=O)CCC1N1C(=O)c2ccc(Br)c3cccc1c23] | Compound 46 |
| 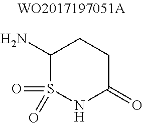 | 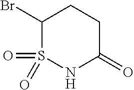 | 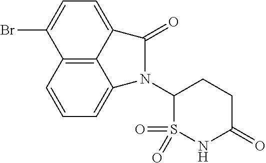 [SMILES: O=C1CCC(N2C(=O)c3ccc(Br)c4cccc2c34)S(=O)(=O)N1] | Compound 47 |
| 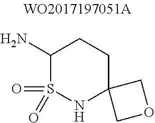 [SMILES: NC1CCC2(COC2)NS1(=O)=O] | 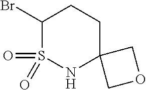 [SMILES: O=S1(=O)NC2(CCC1Br)COC2] | 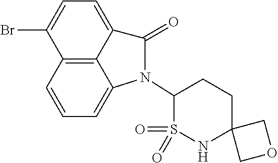 [SMILES: O=C1c2ccc(Br)c3cccc(c23)N1C1CCC2(COC2)NS1(=O)=O] | Compound 48 |
| 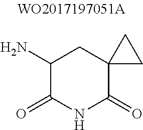 [SMILES: NC1CC2(CC2)C(=O)NC1=O] | 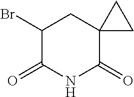 [SMILES: O=C1NC(=O)C2(CC2)CC1Br] | 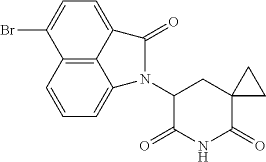 [SMILES: O=C1NC(=O)C2(CC2)CC1N1C(=O)c2ccc(Br)c3cccc1c23] | Compound 49 |

### Example 35. Synthesis of 3-(5-bromo-2-oxobenzo[cd]indol-1(2H)-yl)azepane-2,7-dione (Compound 50)

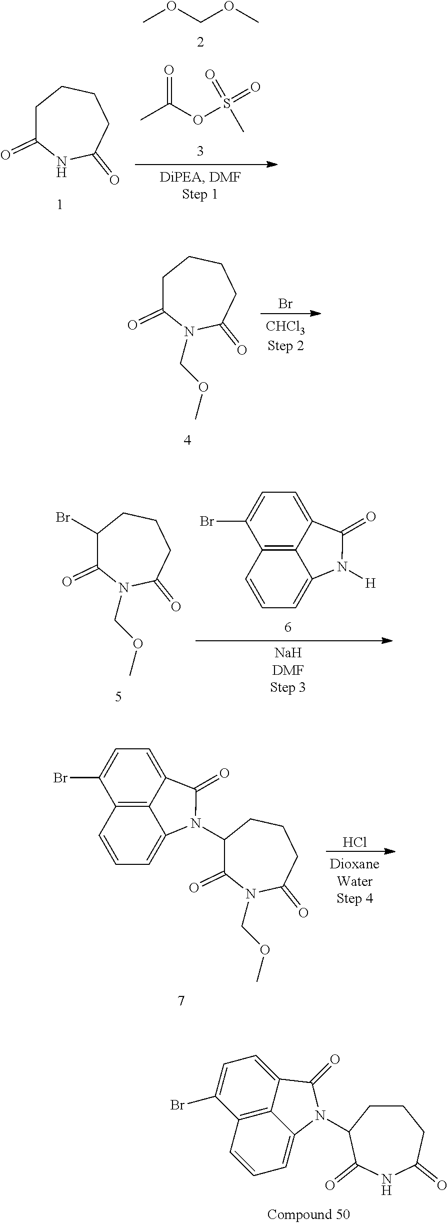

Step 1: A solution of dimethoxymethane 2 (4 eq) was added at 0° (C to acetylmethanesulfonate 3 (4 eq) and the reaction stirred at 25° C. for 2 hours. A solution of 2,7-azepanedione 1 (1 eq, CAS #4726-93-6) and DiPEA (4 eq) in DMF is added to the reaction mixture over 45 min, then stirred for 15 min. Standard work up and purified using standard protocols to afford 1-(methoxymethyl)azepane-2,7-dione 4. (As described in US2003375340)

Step 2: A solution of 1-(methoxymethyl)azepane-2,7-dione 4 (1 eq) and Br₂ (1 eq) in CHCL₃ in a sealed tube is heated at 110° C. for 1.5 hours. Standard workup and purification using standard protocols to afford 3-bromo-1-(methoxymethyl)azepane-2,7-dione 5.

Step 3: To a solution of 3-bromo-1-(methoxymethyl)azepane-2,7-dione 5 (5 eq) in THE at 0° C. is added NaH (60% in oil, 10 eq), in portions, at 0° C. and the reaction mixture is stirred at room temperature for 60 min. The reaction mixture is cooled to 0° C., 3-bromo-1-(methoxymethyl)azepane-2,7-dione (1 eq) in THE is added slowly, the cooling bath removed and the reaction mixture is slowly heated to 65° C., and the reaction mixture is stirred at this temperature until the reaction is judged complete. A standard workup and purification using standard protocols to afford 3-(5-bromo-2-oxobenzo[cd]indol-1(2H)-yl)-1-(methoxymethyl)azepane-2,7-dione 7.

Step 4: To a solution of 3-(5-bromo-2-oxobenzo[cd]indol-1(2H)-yl)-1-(methoxymethyl)azepane-2,7-dione 7 (1 eq) in Dioxane, water and conc. HCl is heated at reflux until the reaction is judged complete. A standard workup and purification using standard protocols to afford 3-(5-bromo-2-oxobenzo[cd]indol-1(2H)-yl)azepane-2,7-dione Compound 50.

### Example 36. Synthesis of 3-(5-bromo-2-oxobenzo[cd]indol-1(2H)-yl)pyrrolidine-2,5-dione (Compound 50)

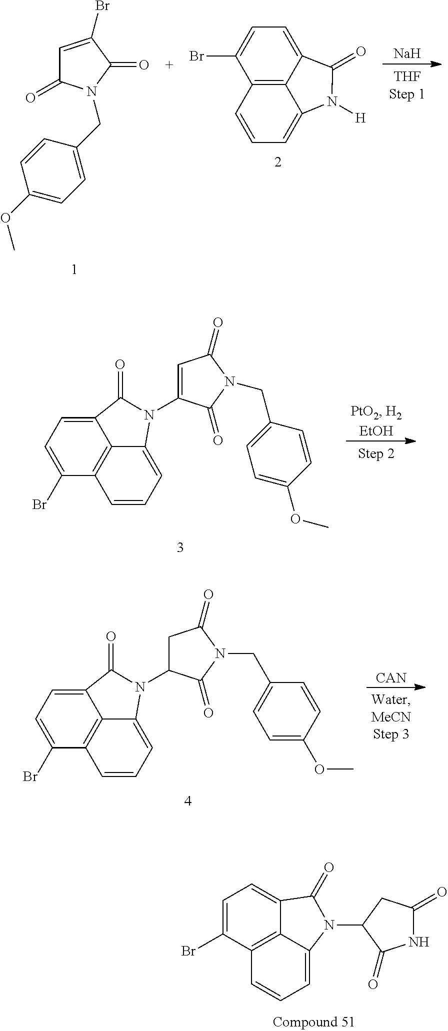

Step 1: To a solution of 3-bromo-1-(methoxymethyl)azepane-2,7-dione 2 (5 eq) in THE at 0° C. is added NaH (60% in oil, 10 eq), in portions, at 0° C. and the reaction mixture is stirred at room temperature for 60 min. The reaction mixture is cooled to 0° C., 3-bromo-1-{[4-(methyloxy)phenyl]methyl}-1H-pyrrole-2,5-dione 1 (WO2008074716, 1 eq) in THE is added slowly, the cooling bath removed and the reaction mixture is slowly heated to 65° C., and the reaction mixture is stirred at this temperature until the reaction is judged complete. A standard workup and purification to afford 3-(5-bromo-2-oxobenzo[cd]indol-1(2H)-yl)-1-(4-methoxybenzyl)-1H-pyrrole-2,5-dione 3.

Step 2: To a suspension of 3-(5-bromo-2-oxobenzo[cd]indol-1(2H)-yl)-1-(4-methoxybenzyl)-1H-pyrrole-2,5-dione 3 and catalytic PtO₂ in in EtOH, is a stirred under an atmosphere of hydrogen, at appropriate pressure and temperature, to afford 3-(5-bromo-2-oxobenzo[cd]indol-1(2H)-yl)-1-(4-methoxybenzyl)pyrrolidine-2,5-dione 4 after standard workup protocols.

Step 3: To a solution of 3-(5-bromo-2-oxobenzo[cd]indol-1(2H)-yl)-1-(4-methoxybenzyl)pyrrolidine-2,5-dione 4 in acetonitrile water, is added CAN (1-3 eq) and stirred at room temperature until the reaction is judged complete. Standard workup and purification using standard protocols to afford 3-(5-bromo-2-oxobenzo[cd]indol-1(2H)-yl)pyrrolidine-2,5-dione Compound 51.

### Example 37. Synthesis of [1-(2,6-dioxo-3-piperidyl)-2-oxo-benzo[cd]indol-3-yl]trifluoromethanesulfonate (Compound 52)

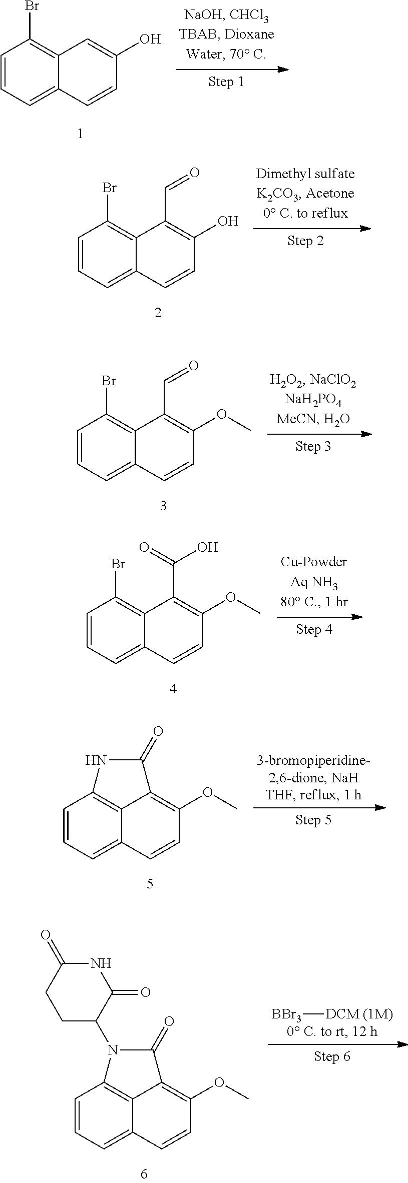

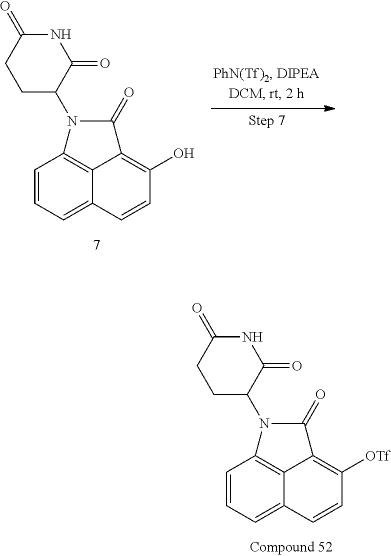 [SMILES: O=C1CCC(N2C(=O)c3c(O)ccc4cccc2c34)C(=O)N1 ; O=C1CCC(N2C(=O)c3c(OS(=O)(=O)C(F)(F)F)ccc4cccc2c34)C(=O)N1]

Step 1: 8-bromonaphthalen-2-ol 1 (5.0 g, 22.41 mmol) was dissolved in 40% NaOH solution (9 mL) and heated until a homogeneous mix formed. Then the temperature of the reaction mixture was lowered to 75-80° C. and tetrabutylammonium bromide (252.90 mg, 784.52 mmol) was added along with 1,4-dioxane (2.9 mL) and IPA (0.1 mL) with stirring at the same temperature. Chloroform (4.01 g, 33.62 mmol, 2.69 mL) was added dropwise over a period of 1 hr, then reaction mixture was stirred at 75° C. for 6 hr. The reaction mixture was acidified with 1 N HCl solution and extracted with ethyl acetate. The organic layers were washed with brine and dried over anhydrous sodium sulfate and filtered. The filtrate was evaporated under reduced pressure to get crude residue which was further purified by silica gel column chromatography using 10% EtOAc/Hexanes as eluent to afford the pure compound 8-bromo-2-hydroxy-naphthalene-1-carbaldehyde 2 (1.65 g, 6.24 mmol, 27.85% yield) as a pinkish-white solid. ¹H NMR (400 MHz, DMSO-d₆) δ 12.43 (s, 1H), 11.17 (s, 1H), 8.15 (d, J=9.04 Hz, 1H), 7.98 (t, J=6.86 Hz, 2H), 7.36 (t, J=7.72 Hz, 1H), 7.26 (d, J=9.0 Hz, 1H); LC MS. ES+248.9, 250.9 (bromo pattern).

Step 2: To a well-stirred solution of 8-bromo-2-hydroxy-naphthalene-1-carbaldehyde 2 (1.4 g, 5.58 mmol) in acetone (10.0 mL) was added and anhydrous potassium carbonate (K₂CO₃), 99% (1.00 g, 7.25 mmol) while cooled to 0° C. Dimethyl sulfate (843.98 mg, 6.69 mmol, 634.57 mL) was added to the mixture, and the mixture was stirred for 30 min at room temperature then refluxed for 16 h. The reaction mixture was then cooled to room temperature and filtered through a bed of celite. The filtrate was then dried in vacuo, extracted with ethyl acetate and washed with water. The organic solvent was separated, dried over anhydrous sodium sulfate, filtered and concentrated in vacuo to get semisolid material which was then purified by silica gel column chromatography to get pure 8-bromo-2-methoxy-naphthalene-1-carbaldehyde 3 (1.41 g, 5.21 mmol, 93.48% yield) as a white solid. ¹H NMR (400 MHz, DMSO-d₆) δ 11.06 (s, 1H), 8.17 (d, J=9.08 Hz, 1H), 8.01 (d, J=7.92 Hz, 1H), 7.89 (d, J=7.32 Hz, 1H), 7.62 (d, J=9.08 Hz, 1H), 7.33 (t, J=7.76 Hz, 1H), 3.92 (s, 3H); LC MS: ES+264.95, 266.97 (bromo pattern)

Step 3: To a well-stirred solution of 8-bromo-2-methoxy-naphthalene-1-carbaldehyde 3 (1.5 g, 5.66 mmol) in ACN (5.65 mL) was added sodium dihydrogen phosphate monohydrate (179.58 mg, 1.30 mmol) in water (2.25 mL), after stirring for 5 min at room temperature 50% hydrogen peroxide (577.39 mg, 8.49 mmol, 524.90 μL) was added dropwise and the reaction mix was stirred for additional 10 min at the same temperature, after which time sodium chlorite (921.12 mg, 10.18 mmol) in water (0.9 mL) was added dropwise for a period of 30 min. The reaction mix was allowed to stir at room temperature overnight. The reaction mix was cooled to 0° C. and acidified by 1 N HCl dropwise and extracted with 5% MeOH/DCM. The organic portion was washed with brine solution then dried over anhydrous sodium sulfate and filtered. The filtrate was evaporated to dryness under reduced pressure to get a crude residue which was then washed with pentane to get the pure compound 8-bromo-2-methoxy-naphthalene-1-carboxylic acid 4 (1.0 g, 3.16 mmol, 55.92% yield) as a yellow solid. ¹H NMR (400 MHz, DMSO-d₆) δ 13.06 (br s, 1H), 8.09 (d, J=9.08 Hz, 1H), 7.98 (d, J=7.92 Hz, 1H), 7.87 (d, J=7.24 Hz, 1H), 7.59 (d, J=9.08 Hz, 1H), 7.28 (t, J=7.76 Hz, 1H), 3.92 (s, 3H); LC MS: 263.1, 265.0 (bromo pattern)

Step 4: To a stirred suspension of 8-bromo-2-methoxy-naphthalene-1-carboxylic acid 4 (1.35 g, 4.80 mmol) in ammonium hydroxide (0.5 mL) in a 2-necked round bottom flask, copper powder (79.35 mg, 1.25 mmol) was added and the reaction mixture was stirred at 80° C. for 2 hr. The reaction mixture was acidified with concentrated hydrochloric acid and the resulting yellow suspension was extracted with ethyl acetate, dried over anhydrous sodium sulfate, and filtered. The filtrate was evaporated under reduced pressure to afford the crude which was then washed with 10% ether/pentane to get the pure compound 3-methoxy-1H-benzo[cd]indol-2-one 5 (941 mg, 4.16 mmol, 86.56% yield) as a brown solid. ¹H NMR (400 MHz, DMSO-d₆) δ 10.62 (s, 1H), 8.15 (d, J=8.84 Hz, 1H), 7.51-7.46 (m, 2H), 7.33 (t, J=7.68 Hz, 1H), 6.94 (d, J=7.04 Hz, 1H), 4.13 (s, 3H); LC MS: ES+200.36.

Step 5: To a well-stirred solution of 3-methoxy-1H-benzo[cd]indol-2-one 5 (20 g, 100.40 mmol) in dry THE (50.0 mL) was added sodium hydride (60% dispersion in mineral oil, 38.47 g, 1.00 mol) portionwise with cooling, maintaining the temp <5° C. Following completion of the addition, the resultant mixture was stirred for 15 minutes at room temperature. The reaction mixture was then cooled again to 0° C. and 3-bromopiperidine-2,6-dione (96.39 g, 502.00 mmol) was added portionwise. After complete addition, the resulting solution was heated to 70° C. for 1 hr. After which time, the reaction mixture was cooled to 0° C. and quenched with the addition of ice cooled water. Aqueous layer was extracted with ethyl acetate (3×500 mL). Combined organic layers were dried over anhydrous sodium sulfate, filtered and concentrated under reduced pressure to get a crude residue of 3-(3-methoxy-2-oxo-benzo[cd]indol-1-yl)piperidine-2,6-dione 6 (18 g, 48.46 mmol, 48.27% yield) as an off-white solid which was used for the next step reaction without further purification. LC MS: ES+311.18.

Step 6: 3-(3-methoxy-2-oxo-benzo[cd]indol-1-yl)piperidine-2,6-dione 6 (18 g, 58.01 mmol) was added to a round bottom flask and tribromoborane (1 M, 580.08 mL) was added under cooling to 0° C. After complete addition of tribromoborane, the reaction mixture was stirred at the same temperature for 10-20 minutes, then allowed to warm to room temperature slowly. The reaction mixture was allowed to stir at room temperature overnight. The reaction mixture was diluted with DCM, poured into ice-water, and extracted with more DCM (3×300 mL). The organic solvent was dried over anhydrous sodium sulfate, filtered, and evaporated under reduced pressure to get a crude residue of 7 (15 g, 28.64 mmol, 49.37% yield) as a brown solid which was used for the next reaction without further purification. LC MS: ES+297.17.

Step 7: To a stirred solution of 3-(3-hydroxy-2-oxo-benzo[cd]indol-1-yl)piperidine-2,6-dione 7 (15 g, 50.63 mmol) in DMF (30.0 mL) was added triethylamine (10.25 g, 101.26 mmol, 14.11 mL) dropwise under cooling to 0° C. and stirred at 0° C. for 20 min. Then 1,1,1-trifluoro-N-phenyl-N-(trifluoromethylsulfonyl)methanesulfonamide (19.90 g, 55.69 mmol) was added and the reaction mix stirred at room temperature for 30 min. The reaction mix was quenched with crushed ice and extracted with ethyl acetate. The organic layer was again washed with brine, dried over anhydrous sodium sulfate, filtered, and evaporated to dryness to get a crude residue. This residue was purified by silica gel column chromatography using 5-10% ethyl acetate/DCM as eluent to obtain the pure compound [1-(2,6-dioxo-3-piperidyl)-2-oxo-benzo[cd]indol-3-yl]trifluoromethanesulfonate Compound 52 (15.5 g, 23.45 mmol, 46.32% yield) as a yellow solid. LC MS: ES+429.17.

### Example 38. Approach 1 to synthesis of 1-(2,6-dioxo-3-piperidyl)-2-oxo-benzo[cd]indole-3-carbaldehyde (Compound 53)

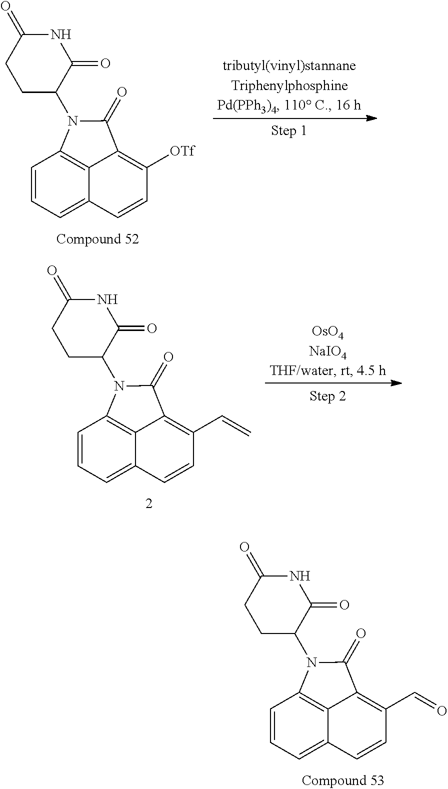 [SMILES: O=C1CCC(N2C(=O)c3c(OS(=O)(=O)C(F)(F)F)ccc4cccc2c34)C(=O)N1 ; "tributyl(vinyl)stannaneTriphenylphosphinePd(PPh3)4, 110° C., 16 h" ; C=Cc1ccc2cccc3c2c1C(=O)N3C1CCC(=O)NC1=O ; "OsO4NaIO4THF/water, rt, 4.5 h" ; O=Cc1ccc2cccc3c2c1C(=O)N3C1CCC(=O)NC1=O]

Step 1: A well-stirred solution of (1-(2,6-dioxo-3-piperidyl)-2-oxo-benzo[cd]indol-3-yl]trifluoromethanesulfonate, Compound 52, (15.5 g, 36.19 mmol) in dioxane (100 mL) was degassed under argon atmosphere for 15 minutes followed by the addition of tributyl(vinyl)stannane (14.92 g, 47.04 mmol, 13.69 mL), triphenylphosphine (474.57 mg, 1.81 mmol) and tetrakis(triphenylphosphine)palladium (2.09 g, 1.81 mmol). The reaction mixture was heated to 110° C. for 16 hours. After completion of the reaction, the reaction mixture was filtered through a pad of celite and washed with ethyl acetate several times. The filtrate was washed with water and brine then the organic solvent was separated. The organic solvent was then dried over anhydrous sodium sulfate, filtered, and evaporated to dryness to get a crude residue which was purified by flash chromatography using 0-2% MeOH in DCM to afford 3-(2-oxo-3-vinyl-benzo[cd]indol-1-yl)piperidine-2,6-dione 2 (7 g, 21.48 mmol, 59.36% yield) as a yellow solid. LC MS: ES+307.2.

Step 2: To a stirred solution of 3-(2-oxo-3-vinyl-benzo[cd]indol-1-yl)piperidine-2,6-dione 2 (7.5 g, 24.48 mmol) in water (10 mL) and THE (30 mL) was added osmium tetroxide (124.49 mg, 489.69 μmol) and the reaction mixture was stirred at room temperature for 20 minutes before addition of sodium periodate (13.09 g, 61.21 mmol). The reaction mixture was further stirred at room temperature for 4 hours. After completion of the reaction, the reaction mixture was diluted with ethyl acetate (200 mL) and washed with water and brine. The organic layer was separated, dried over anhydrous sodium sulfate and evaporated under reduced pressure to get crude residue which was purified by flash chromatography using 0-5% MeOH-DCM to afford 1-(2,6-dioxo-3-piperidyl)-2-oxo-benzo[cd]indole-3-carbaldehyde Compound 53 (7 g, 20.44 mmol, 83.46% yield) as a yellow solid. ¹H NMR (400 MHz, DMSO-d₆) δ 11.17 (s, 1H), 10.94 (s, 1H), 8.34 (d, 1H, J=8.2 Hz), 8.15 (d, 1H, J=8.4 Hz), 7.66-7.75 (m, 2H), 7.27 (d, 1H, J=7.0 Hz), 5.50 (dd, J=12.52, 5.12 Hz, 1H), 2.92-2.99 (m, 1H), 2.67-2.79 (m, 2H), 2.12-2.16 (m, 1H).

### Example 39. Approach 2 to synthesis of 1-(2,6-dioxo-3-piperidyl)-2-oxo-benzo[cd]indole-3-carbaldehyde (Compound 53)

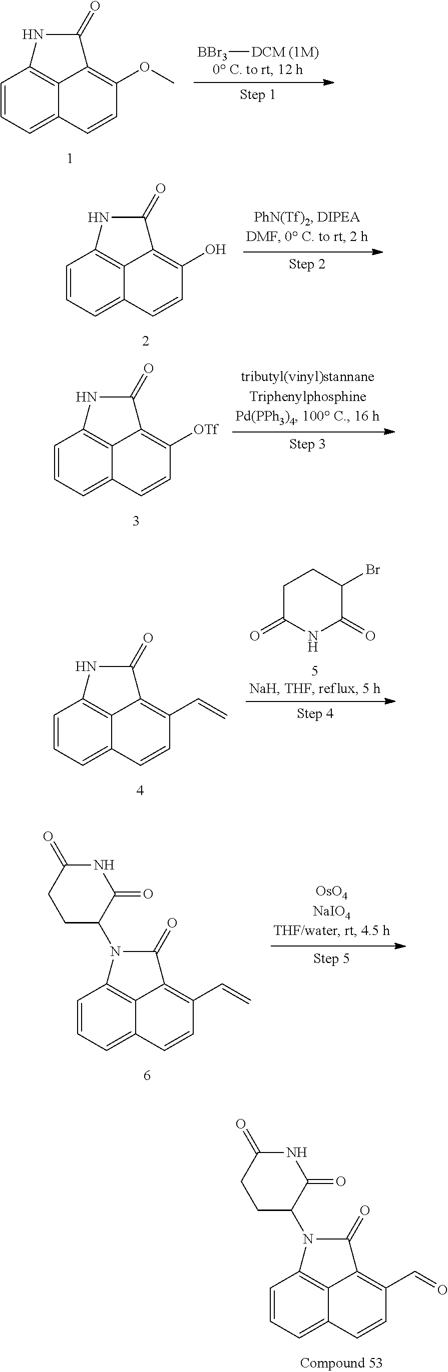

Step 1: To a well-stirred solution of 3-methoxybenzo[cd]indol-2(1H)-one (1) (5.5 g, 27.609 mmol) in DCM (20 mL) was added tribromoborane (72.66 g, 290.04 mmol) slowly under cooling to 0° C. After complete addition of tribromoborane, the reaction mixture was stirred at the same temperature for 10-20 minutes. The reaction mixture was allowed to warm to room temperature slowly and the reaction mixture was continued to stirred at rt for 12 hr. After completion of the reaction, the reaction was diluted with DCM, poured into ice water, and extracted with DCM (3×300 mL). The organic solvent was dried over anhydrous sodium sulfate, filtered, and filtrate evaporated under reduced pressure to get a crude residue of 3-hydroxybenzo[cd]indol-2(1H)-one (2) (4.2 g, 22.896 mmol, 82.16% yield) as a brown solid which was used for the next step reaction without further purification. LC MS: ES+186.2.

Step 2: To a well-stirred solution of 3-hydroxy-1H-benzo[cd]indol-2-one (2) (500 mg, 2.70 mmol) in DMF (1.0 mL) was added N,N-Diisopropylethylamine (697.94 mg, 5.40 mmol, 940.62 μL) dropwise under cooling to 0° C. then stirred at 0° C. for 20 mins. After that, 1,1,1-trifluoro-N-phenyl-N-(trifluoromethylsulfonyl)methanesulfonamide (964.61 mg, 2.70 mmol) was added and stirred at rt for 1.5 hr. After complete consumption of starting material, the reaction mix was quenched with crushed ice and extracted with ethyl acetate. The organic layer was again washed with brine solution, separated, dried over anhydrous sodium sulfate, filtered and concentrated under reduced pressure to get a crude residue which was then purified by silica gel column chromatography using 10-15% ethyl acetate/hexanes as eluent to get the pure compound (2-oxo-1H-benzo[cd]indol-3-yl) trifluoromethanesulfonate 3 (570 mg, 1.69 mmol, 62.55% yield) as a yellow solid. LC MS: ES+317.8.

Step 3: A well-stirred solution of (2-oxo-1H-benzo[cd]indol-3-yl) trifluoromethanesulfonate 3 (2.65 g, 8.35 mmol) in dioxane (30 mL) was degassed under argon atmosphere for 15 minutes followed by the addition of tributyl(vinyl)stannane (3.44 g, 10.86 mmol, 3.16 mL), triphenylphosphine (109.55 mg, 417.66 μmol) and tetrakis(triphenylphosphine)palladium (482.64 mg, 417.66 μmol). The reaction mixture was heated and stirred at 110° C. for 16 hours. After completion of the reaction, the reaction mixture was filtered through a pad of celite and washed with ethyl acetate several times. The filtrate was washed with water and brine and the organic layer was separated. The organic solvent was then dried over anhydrous sodium sulfate, filtered, and evaporated to dryness to get a crude residue. The crude residue was purified by flash chromatography using 0-2% MeOH in DCM to afford 3-vinyl-1H-benzo[cd]indol-2-one 4 (1.5 g, 90% yield) as a yellow solid. LC MS: ES+196.0.

Step 4: To a cooled solution of 3-vinyl-1H-benzo[cd]indol-2-one 4 (2.3 g, 11.78 mmol) in dry THE (20 mL), sodium hydride (60% dispersion in mineral oil, 4.71 g, 117.82 mmol) was added portionwise, maintaining the temp <5° C. Once the addition was over, the resultant mixture was stirred for 30 minutes at RT. The reaction mixture was then cooled again to 0° C. and 3-bromopiperidine-2,6-dione (5) (11.31 g, 58.91 mmol) was added to the mixture portionwise. After complete addition, the resulting solution was refluxed for 4 hr. After complete consumption of starting material, the reaction mixture was cooled to RT and poured into ice water. The aqueous portions were extracted with ethyl acetate (3×100 mL), and the combined organic solvents were dried over anhydrous sodium sulfate, filtered, and evaporated under reduced pressure to get a crude residue. The residue was washed with pentane and then dried under reduced pressure to afford the crude 3-(2-oxo-3-vinyl-benzo[cd]indol-1-yl)piperidine-2,6-dione (6) (3.0 g, 9.794 mmol, 83.28% yield) which was used for the next step without further purification. LC MS: ES+307.4.

Step 5: To a stirred solution of 3-(2-oxo-3-vinyl-benzo[cd]indol-1-yl)piperidine-2,6-dione (6) (1 g, 3.26 mmol) in water (6 mL) and THE (18 mL) was added osmium tetroxide (16.60 mg, 65.29 μmol, 0.3 mL), and the reaction mixture was stirred at room temperature for 20 minutes. Sodium periodate (1.75 g, 8.16 mmol) was then added to the reaction mixture and the reaction was further stirred at room temperature for 4 hours. After completion of the reaction, the reaction mixture was diluted with ethyl acetate (25 mL) and washed with water and brine. The organic layer was separated, dried over anhydrous sodium sulfate, and evaporated under reduced pressure to afford a crude residue. The crude residue was purified by flash chromatography using 0-5% MeOH-DCM to afford 1-(2,6-dioxo-3-piperidyl)-2-oxo-benzo[cd]indole-3-carbaldehyde, Compound 53, (900 mg, 2.63 mmol, 80.48% yield) as a yellow solid. ¹H NMR (400 MHz, DMSO-d₆) δ 11.17 (s, 1H), 10.94 (s, 1H), 8.34 (d, 1H, J=8.4 Hz), 8.15 (d, 1H, J=8.4 Hz), 7.66-7.75 (m, 2H), 7.27 (d, 1H, J=7.0 Hz), 5.49 (dd, J=12.88, 5.32 Hz, 1H), 2.92-2.97 (m, 1H), 2.76-2.80 (m, 1H), 2.66-2.70 (m, 1H), 2.13-2.17 (m, 1H); LC MS: ES+309.1.

### Example 40. Synthesis of 4-bromobenzo[cd]indol-2(1H)-one (Compound 54)

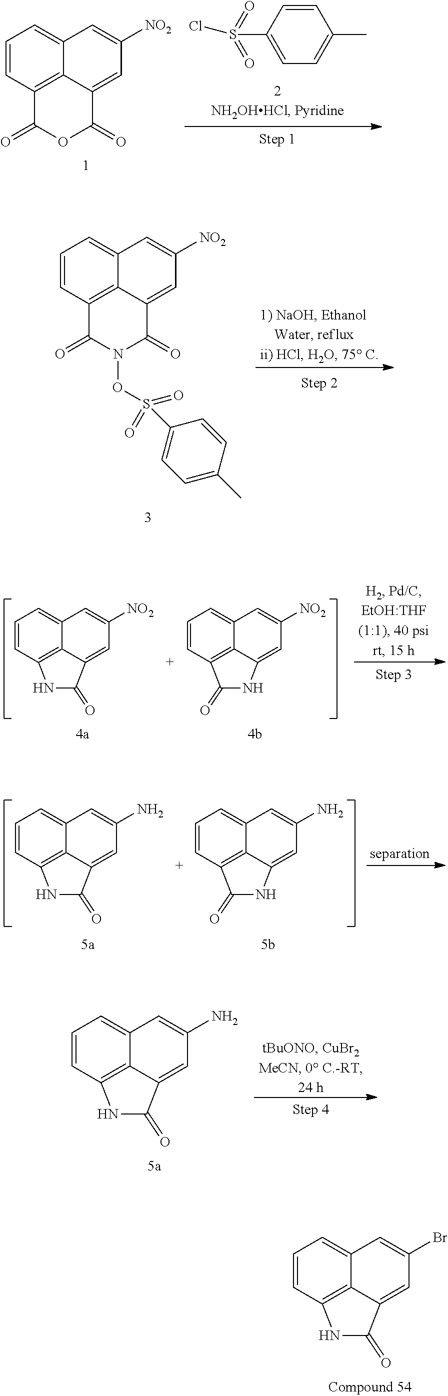

Step 1: 5-nitro-1H,3H-benzo[de]isochromene-1,3-dione 1 (360.0 g, 1480.45 mmol) and hydroxylamine hydrochloride (103.0 g, 1482.23 mmol) were dissolved in pyridine (3.6 L) and the reaction mixture was refluxed for 1 hr. Then the reaction mixture was cooled to 80° C. and p-toluenesulfonyl chloride (564.5 g, 2960.92 mmol) was added portionwise, again refluxing for an additional 2 hrs. After completion of the reaction, the reaction mixture was cooled to RT and poured into ice water (6 L) and stirred. The resulting precipitate was filtered and rinsed with additional cold water and saturated aqueous NaHCO₃ solution to afford the pure compound 5-nitro-1,3-dioxo-1H-benzo[de]isoquinolin-2(3H)-yl 4-methylbenzenesulfonate 3 (210.0 g, 0.51 mol, 34.4% yield) as a yellowish solid. ¹H NMR (d₆-DMSO, 400 MHz) δ 9.55 (s, 1H), 8.91 (s, 1H), 8.85 (d, J=8.2 Hz, 1H), 8.67 (d, J=7.2 Hz, 1H), 8.1 (m, 1H), 7.94 (d, J=8.2 Hz, 1H), 7.52 (d, J=4.2 Hz, 1H), 2.48 (s, 3H); LC MS: ES+413.4.

Step 2: 5-nitro-1,3-dioxo-1H-benzo[de]isoquinolin-2(3H)-yl 4-methylbenzenesulfonate 3 (350.4 g, 848.75 mmol) was dissolved in ethanol (2.0 L), water (1.6 L) and aqueous solution of sodium hydroxide (2.7 M, 850 mL) at RT. Then the reaction mixture was refluxed for 1 hr. Then ethanol was removed under reduced pressure. Then aqueous remaining reaction mixture was heated to 75° C. and acidified with concentrated hydrochloric acid. A yellowish compound precipitated, which was filtered to give a residue which was washed with cold water 3 times, then collected and dried under reduced pressure to afford a mixture of two isomers, 4-nitrobenzo[cd]indol-2(1H)-one (4a) and 7-nitrobenzo[cd]indol-2(1H)-one (4b) (180.0 g, 88% purity, determined by LC-MS) as yellowish solid.

### LC MS: ES− 212.6

Step 3: To a degassed solution of the mixture of 4-nitrobenzo[cd]indol-2(1H)-one (4a) and 7-nitrobenzo[cd]indol-2(1H)-one (4b) (250.0 g, 1167.24 mmol) in THF:EtOH (1:1) (1.5 L), Pd/C (10%, wet, 40.0 g) was added and the resulting reaction mixture was subjected to hydrogenation (in Parr-Shaker instrument) under 40 psi for 16 hrs. After completion of the reaction, the mixture was filtered through celite and washed with THE until no compound remained in the celite. Filtrate was collected and evaporated to dryness to afford a crude material, which was purified by silica gel column chromatography using 4.5% THE in DCM as eluting solvent to afford pure 4-aminobenzo[cd]indol-2(1H)-one (5a) (50.0 g, 271.44 mmol, 23.3% yield, 99% purity, indicated by LC-MS) as a yellowish orange solid. ¹H NMR (d₆-DMSO, 400 MHz) δ 10.46 (s, 1H), 7.35 (s, 1H), 7.28-7.21 (m, 2H), 7.06 (s, 1H), 6.61 (d, J=6.5 Hz, 1H), 5.74 (s, 2H); LC MS: ES+185.15

Step 4: To a stirred solution of 4-aminobenzo[cd]indol-2(1H)-one 5a (10 g, 54.35 mmol) in acetonitrile (140 mL) was added tert-butyl nitrite (9.68 mL, 81.4 mmol) dropwise at room temperature over 20 minutes. The reaction mixture turned dark red and was then cooled to 0° C. before CuBr₂ (12.14 g, 54.35 mmol) was added portionwise over 30 minutes. Reaction mixture was allowed to warm to room temperature and stirred for 24 hours. Reaction mixture was diluted with THE (200 mL) and filtered through a pad of celite. The celite pad was washed with THF (3×200 mL) and the combined filtrate was evaporated under reduced pressure to obtain brownish crude. Crude material was purified by silica gel column chromatography using 0-10% THF/DCM to afford 4-bromobenzo[cd]indol-2(1H)-one, Compound 54, (4.5 g, 36%) as a yellow solid. ¹H NMR (d₆-DMSO, 400 MHz) δ 10.88 (s, 1H), 8.45 (s, 1H), 8.10 (s, 1H), 7.59-7.50 (m, 2H), 7.00 (d, J=6.36 Hz, 1H);

### Example 41. Synthesis of 1-(2,6-dioxo-3-piperidyl)-2-oxo-benzo[cd]indole-4-carbaldehyde (Compound 55)

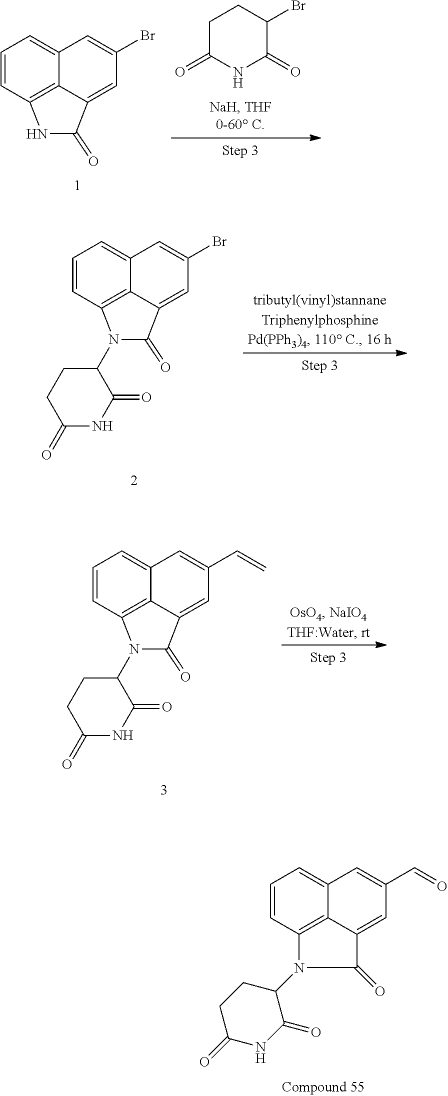

Step 1: To a stirred solution of 4-bromo-1H-benzo[cd]indol-2-one 1 in THE (500 mL) under nitrogen atmosphere at 0° C., sodium hydride (60% dispersion in mineral oil, 15.45 g, 403.10 mmol) was added portionwise over a period of 1 hr. The resulting mixture was allowed to stir at RT for 15 minutes and cooled back to 0° C. before the portion wise addition of 3-bromopiperidine-2,6-dione (38.70 g, 201.55 mmol) over a period of 1 hr. The resulting mixture was again allowed to warm at RT and then heated to 95° C. for 1 hr. After completion of the reaction, the mixture was diluted with EtOAc (1000 mL) and portionwise poured into ice cooled water (500 mL). The aqueous layer was then extracted with EtOAc (1000 mL) then the combined organic layers successively washed with water (1000 mL), brine (500 mL), dried over anhydrous sodium sulfate, filtered, and concentrated under reduced pressure to afford a yellowish crude compound. Triturating the crude compound with Et₂O rendered the 3-(4-bromo-2-oxobenzo[cd]indol-1(2H)-yl)piperidine-2,6-dione (2) (12 g, 83% yield) as yellowish solid. ¹H NMR (d₆-DMSO, 400 MHz) δ 11.14 (s, 1H), 8.52 (s, 1H), 8.23 (s, 1H), 7.65-7.55 (m, 2H), 7.22-7.19 (m, 1H), 5.47-5.44 (m, 1H), 2.96-2.90 (m, 1H), 2.77-2.63 (m, 2H), 2.13-2.11 (m, 1H); LC MS: ES+358.9, 361.1.

Step 2: The stirred solution of 3-(4-bromo-2-oxo-benzo[cd]indol-1-yl)piperidine-2,6-dione 2 (8.3 g, 23.11 mmol) in 1,4-dioxane (160 mL) was degassed under argon atmosphere for 15 minutes followed by addition of tributyl(vinyl)stannane (9.53 g, 30.04 mmol, 8.82 mL), triphenylphosphane (606.11 mg, 2.31 mmol) and tetrakis(triphenylphosphine)palladium (1.34 g, 1.16 mmol). The resulting solution was then heated at 110° C. for 16 hours. After completion of the reaction, the reaction mixture was filtered through a pad of celite and washed with ethyl acetate (3×100 mL). The filtrate was washed with water (200 mL), brine (200 mL) and organic layers separated and dried over anhydrous sodium sulfate. Combined organic layers were evaporated under reduced pressure to obtain a yellowish crude residue which was triturated with Et₂O to yield 3-(2-oxo-4-vinylbenzo[cd]indol-1(2H)-yl)piperidine-2,6-dione 3 (8 g crude) as a yellowish solid. ¹H NMR (d₆-DMSO, 400 MHz) δ 11.15 (s, 1H), 8.32 (s, 1H), 8.25 (s, 1H), 7.64 (d, J=8.4 Hz, 1H), 7.52 (t, J=7.76 Hz, 1H), 7.13 (d, J=7.12 Hz, 1H), 7.09-7.02 (m, 1H), 6.17 (m, 1H), 5.47-5.43 (m, 2H), 3.00-2.91 (m, 1H), 2.82-2.63 (m, 2H), 2.13-2.10 (m, 1H); LC MS: ES+307.3.

Step 3: To a stirred solution of crude 3-(2-oxo-4-vinyl-benzo[cd]indol-1-yl)piperidine-2,6-dione 3 (8 g, 26.12 mmol) in water (17 mL) and THE (51 mL) was added osmium tetroxide (132.79 mg, 522.34 μmol) and the reaction mixture was stirred at room temperature for 20 minutes followed by the addition of sodium periodate (13.97 g, 65.29 mmol). The reaction mixture was stirred further at room temperature for 4 hours. After completion of the reaction the reaction mixture was diluted with ethyl acetate (250 mL) and washed with water (200 mL) and brine (200 mL). Organic portion was separated, dried over anhydrous sodium sulfate and concentrated to afford a crude material which was triturated with Et₂O to afford 1-(2,6-dioxo-3-piperidyl)-2-oxo-benzo[cd]indole-4-carbaldehyde Compound 55 as a yellow solid (7 g, 98%, over 2 steps). ¹H NMR (d₆-DMSO, 400 MHz) δ 11.16 (s, 1H), 10.29 (s, 1H), 8.88 (s, 1H), 8.45 (s, 1H), 7.87 (d, J=8.44 Hz, 1H), 7.66 (t, J=7.8 Hz, 1H), 7.33 (d, J=7.16 Hz, 1H), 5.50 (dd, J=12.8, 5.36 Hz, 1H), 2.96-2.91 (m, 1H), 2.82-2.64 (m, 2H), 2.14-2.12 (m, 1H); LC MS: ES+309.2.

### Example 42. Synthesis of 3-(4-amino-2-oxo-benzo[cd]indol-1-yl)piperidine-2,6-dione (Compound 56)

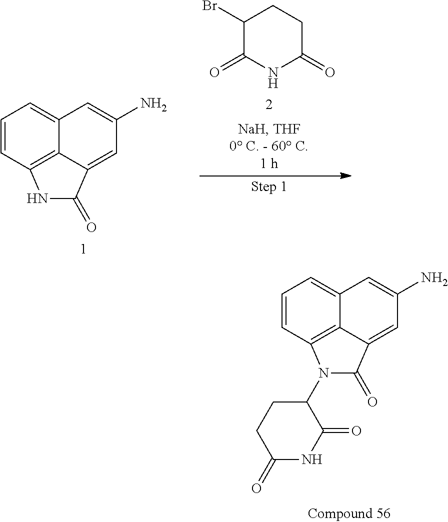 [SMILES: O=C1CCC(Br)C(=O)N1 ; Nc1cc2c3c(cccc3c1)NC2=O ; Nc1cc2c3c(cccc3c1)N(C1CCC(=O)NC1=O)C2=O]

Step 1: To a stirred solution of 4-amino-1H-benzo[cd]indol-2-one 1 (3 g, 16.29 mmol) in THE (200 mL) was added sodium hydride (60% dispersion in mineral oil, 4.69 g, 195.45 mmol) portionwise at 0° C. After the completion of the addition the reaction mixture was stirred at room temperature for 10 min before the portionwise addition of 3-bromopiperidine-2,6-dione 2 (15.64 g, 81.44 mmol) at 0° C. The resulting reaction mixture was refluxed at 70° C. for 1 hour. After completion of the reaction, the reaction mixture was diluted with ethyl acetate and poured into ice water. The organic layer was washed with water, separated, dried over anhydrous sodium sulfate and evaporated under reduced pressure to obtain the crude material. This crude material was triturated with ether and pentane to afford 3-(4-amino-2-oxo-benzo[cd]indol-1-yl)piperidine-2,6-dione Compound 56 (2.8 g, 9.48 mmol, 58.22% yield) as a yellow solid. ¹H NMR (d₆-DMSO, 400 MHz) δ 11.11 (s, 1H), 7.43 (s, 1H), 7.31-7.30 (m, 2H), 7.11 (s, 1H), 6.76 (m, 1H), 5.82 (br s, 2H), 5.35-5.32 (m, 1H), 2.95-2.88 (m, 1H), 2.76-2.61 (m, 2H), 2.08-2.05 (m, 1H); LC MS: ES+296.29.

### Example 43. Synthesis of 3-(4-amino-2-oxobenzo[cd]indol-1(2H)-yl)piperidine-2,6-dione (Compound 36) and 5-(4,4,5,5-tetramethyl-1,3,2-dioxaborolan-2-yl)-1H-benzo[cd]indol-2-one (Compound 57)

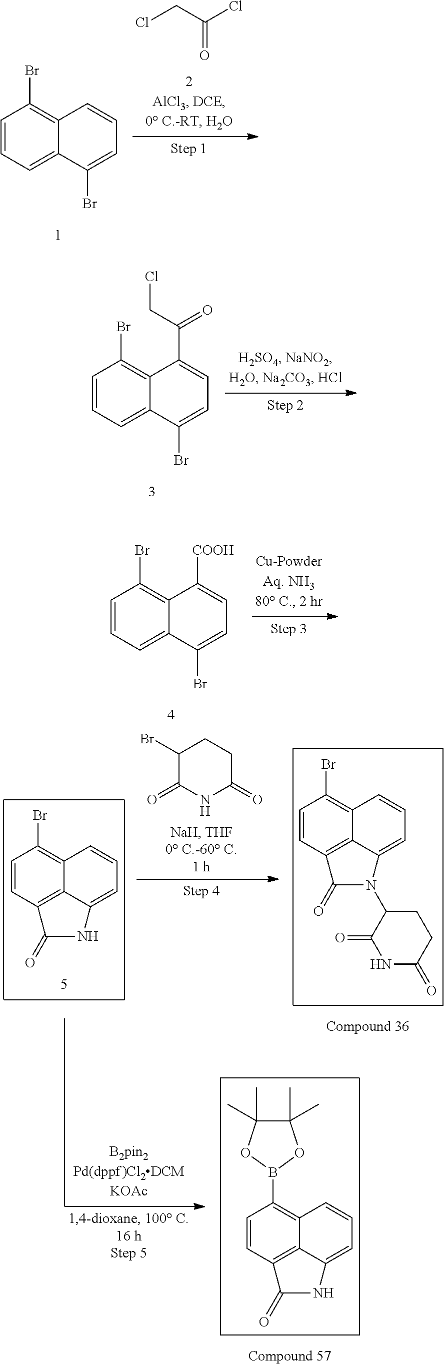

Step 1: To a stirred solution of 1,5-dibromonaphthalene (1) (162 g, 566.51 mmol) in DCE (2000 mL) at 0° C. was added 2-chloroacetyl chloride (2) (83.18 g, 736.46 mmol, 58.57 mL) dropwise. The resultant solution was stirred at 0° C. for 15 minutes followed by portionwise addition of anhydrous aluminum trichloride (98.20 g, 736.46 mmol, 40.25 mL). The resultant reaction mixture was then slowly warmed to RT and stirred for 16 hr. After completion, the reaction mixture was poured into ice water and extracted twice with DCM. The combined organic layers were further washed with water and brine, dried over anhydrous sodium sulfate, filtered, and concentrated under reduced pressure. The crude thus obtained was purified by silica gel column chromatography using 0-5% EtOAc in hexanes to afford 2-chloro-1-(4,8-dibromo-1-naphthyl)ethanone (3) (150 g, 69% yield) as an off white solid. ¹H NMR (d₆-DMSO, 400 MHz) δ 8.36 (dd, J=8.48, 0.72 Hz, 1H), 8.11-8.07 (m, 2H), 7.69 (t, J=8.04 Hz, 1H), 7.59 (d, J=7.8 Hz, 1H), 5.05 (s, 2H);

Step 2: To a stirred solution of 2-chloro-1-(4,8-dibromo-1-naphthyl)ethanone (3) (151 g, 416.62 mmol) in sulfuric acid (1.8 L) was added sodium nitrite (30.27 g, 438.75 mmol) at room temperature and the resultant reaction mixture was stirred at 65° C. for 45 minutes. After completion the reaction mixture was poured into 2 L of cold water and the resulting solid was filtered off. The solid thus obtained was added to 4 L of a 10% sodium carbonate solution and stirred for 30 minutes at room temperature. The mixture was filtered, and the filtrate was cautiously acidified with concentrated hydrochloric acid under vigorous stirring then filtered again to remove insoluble impurities. The aqueous filtrate was then extracted twice with ethyl acetate. The combined organic layers were further washed with brine, dried over anhydrous sodium sulfate and concentrated under reduced pressure to afford 4,8-dibromonaphthalene-1-carboxylic acid (4) (110 g, 72% yield) as a light brown solid. ¹H NMR (d₆-DMSO, 400 MHz) δ 13.48 (br s, 1H), 8.33 (d, J=8.36 Hz, 1H), 8.09 (d, J=7.4 Hz, 1H), 8.01 (d, J=7.72 Hz, 1H), 7.65 (t, J=8.0 Hz, 1H), 7.59 (d, J=7.72 Hz, 1H); LC MS: ES−328.90.

Step 3: To a stirred suspension of 4,8-dibromonaphthalene-1-carboxylic acid (4) (65 g, 196.99 mmol) in 700 mL of aqueous ammonia, was added copper powder (3.25 g, 51.22 mmol) and the resultant reaction mixture was stirred at 80° C. for 2 hours. After completion, the reaction mixture was poured into ice water and was slowly acidified with concentrated hydrochloric acid (to pH-2) under vigorous stirring. The resulting yellow precipitate was filtered off and was further dried under reduced pressure to afford 5-bromo-1H-benzo[cd]indol-2-one (5) (39 g, 77% yield) as a brown solid. ¹H NMR (d₆-DMSO, 400 MHz) δ 10.88 (s, 1H), 8.05 (d, J=7.44 Hz, 1H), 7.88 (d, J=7.4 Hz, 1H), 7.61 (t, J=7.8 Hz, 1H), 7.53 (d, J=8.56 Hz, 1H), 7.04 (d, J=7.0 Hz, 1H); LC MS: ES+248.2, 250.1 (Bromo pattern).

Step 4: To a suspension of 5-bromo-1H-benzo[cd]indol-2-one (5) (25 g, 100.78 mmol) in dry THE (250 mL) was added Sodium hydride (60% dispersion in mineral oil, 38.61 g, 1.01 mol) portion wise, maintaining the temperature of the reaction below 5° C. Following the addition, the resultant mixture was slowly warmed to RT and stirred for 15 minutes. The reaction mixture was again cooled to 0° C. and 3-bromopiperidine-2,6-dione (96.75 g, 503.88 mmol) was added to it portionwise, and the resulting reaction mixture was heated at 70° C. for 1 hr. After completion, the reaction mixture was slowly poured into crushed ice and extracted three times with ethyl acetate. The combined organic layers were dried over anhydrous sodium sulfate and concentrated under reduced pressure. The crude thus obtained was triturated with diethyl ether and pentane to afford the desired compound 3-(5-bromo-2-oxo-benzo[cd]indol-1-yl)piperidine-2,6-dione (Compound 36) (16 g, 34% yield) as a light yellow solid. ¹H NMR (d₆-DMSO, 400 MHz) δ 11.14 (s, 1H), 8.12 (d, J=7.48 Hz, 1H), 7.99 (d, J=7.44 Hz, 1H), 7.72-7.62 (m, 2H), 7.26 (d, J=6.92 Hz, 1H), 5.46 (dd, J=12.84, 5.28 Hz, 1H), 2.99-2.90 (m, 1H), 2.81-2.63 (m, 2H), 2.12-2.07 (m, 1H); LC MS: ES+359.07, 361.02 (Bromo pattern).

Step 5: To a stirred solution of 5-bromo-1H-benzo[cd]indol-2-one (5) (200 mg, 806 μmol) in 1,4 dioxane (10 mL) was added bis(pinacolato)diboron (307 mg, 1.21 mmol) followed by well dried potassium acetate (237 mg, 2.42 mmol, 3 eq). The resultant reaction mixture was degassed with argon for 15 minutes. Following this, Pd(dppf)Cl₂(66 mg, 81 μmol) was then added and the reaction mixture was heated to 100° C. for 16 hrs. After completion of the reaction, the reaction mixture was cooled to RT and filtered through a celite pad and washed with EtOAc. The combined filtrate was then washed with cold water, dried over anhydrous sodium sulfate and concentrated under reduced pressure to afford crude 5-(4,4,5,5-tetramethyl-1,3,2-dioxaborolan-2-yl)-1H-benzo[cd]indol-2-one, Compound 57, (200 mg, 406 μmol, 50% yield) as a crude brown gum which was used without further purification. LC MS: ES+296.2

### Example 44. Synthesis of 5-Chloromethyl-1-(4-methoxy-benzyl)-1H-benzo[cd]indol-2-one (Compound 58)

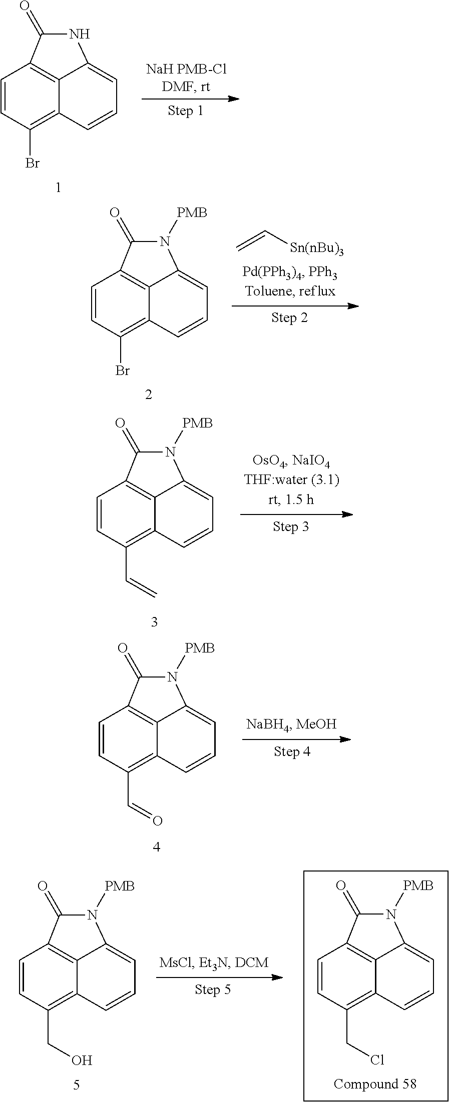

Step 1: To a stirred solution of 5-Bromo-1H-benzo[cd]indol-2-one 1 (50.0 g, 201.532 mmol) in DMF (150 mL) at 0° C. was added sodium hydride (60% dispersion in mineral oil, 7.255 g, 302.297 mmol) and the reaction mixture was stirred at 0° C. for 30 mins. Then 4-methoxy benzyl chloride (32.806 mL, 241.8 mmol) was added and the reaction mixture was slowly warmed to RT and stirred for another 30 min. After completion, the reaction mass was quenched with crushed ice and extracted with EtOAc. The organic layer was further washed with water, brine, dried over anhydrous sodium sulfate and concentrated under reduced pressure. The crude thus obtained was purified by silica gel column chromatography using 0-1% EtOAc in DCM to afford 5-bromo-1-(4-methoxy-benzyl)-1H-benzo[cd]indol-2-one 2 (66 g, 89% Yield) as a yellow solid. ¹H NMR (d₆-DMSO, 400 MHz) δ 8.09 (d, J=7.44 Hz, 1H), 7.98 (d, J=7.44 Hz, 1H), 7.65-7.56 (m, 2H), 7.32 (d, J=8.56 Hz, 2H), 7.19 (d, J=6.96 Hz, 1H), 6.87 (d, J=8.56 Hz, 2H), 5.03 (s, 2H), 3.69 (s, 3H); LC MS: ES+367.80, 369.84

Step 2: A stirred solution of 5-bromo-1-(4-methoxy-benzyl)-1H-benzo[cd]indol-2-one 2 (66 g, 179.348 mmol) in toluene (800 mL) was purged with argon for 20 min. Then tributyl vinyl tin (55.037 mL, 188.315 mmol), triphenylphosphine (2.352 g, 8.967 mmol) and tetrakis(triphenylphosphine)palladium (10.363 g, 8.967 mmol) were added and the reaction mixture was heated to 110° C. for 16 hr. After completion of the reaction, the solvent was evaporated under reduced pressure and the crude thus obtained was purified by silica gel column chromatography using 0-20% EtOAc in hexanes to afford 1-(4-methoxy-benzyl)-5-vinyl-1H-benzo[cd]indol-2-one (3) (45 g, 79% yield) as yellow solid. ¹H NMR (d₆-DMSO, 400 MHz) δ 8.07-8.03 (m, 2H), 7.85 (d, J=8.64 Hz, 1H), 7.59-7.49 (m, 2H), 7.31 (d, J=8.6 Hz, 2H), 7.12 (d, J=7.12 Hz, 1H), 6.87 (d, J=8.56 Hz, 2H), 6.15 (d, J=17.44 Hz, 1H), 5.66 (d, J=11.16 Hz, 1H), 3.69 (s, 3H); LC MS: ES+316.02

Step 3: To a stirred solution of 1-(4-methoxy-benzyl)-5-vinyl-1H-benzo[cd]indol-2-one (3) (45 g, 112.5 mmol) in water (100 mL) and THE (300 mL) was added 4% solution of osmium tetroxide in water (572 mg, 507.35 μmol, 14.3 mL). The reaction mixture was stirred at room temperature for 20 minutes then sodium periodate (60.157 g, 281.25 mmol) was added. The resultant reaction mixture was then stirred at room temperature for 1 hr. After completion of the reaction, the reaction mixture was filtered through a pad of celite and washed with THE and EtOAc. The filtrate collected was then dried over anhydrous sodium sulfate and concentrated under reduced pressure to afford 1-(4-methoxy-benzyl)-2-oxo-1,2-dihydro-benzo[cd]indole-5-carbaldehyde (4) (28 g, 78% yield) as a brown solid. ¹H NMR (d₆-DMSO, 400 MHz) δ 10.48 (s, 1H), 8.41 (d, J=7.12 Hz, 1H), 8.37 (d, J=8.64 Hz, 1H), 8.27 (d, J=7.08 Hz, 1H), 7.65-7.61 (m, 1H), 7.33 (d, J=8.6 Hz, 2H), 7.18 (d, J=7.2 Hz, 1H), 6.88 (d, J=8.6 Hz, 2H), 5.03 (s, 2H), 3.69 (s, 3H); LC MS: ES+317.98

Step 4: To a stirred solution of 1-(4-methoxy-benzyl)-2-oxo-1,2-dihydro-benzo[cd]indole-5-carbaldehyde (4) (28 g, 88.324 mmol) in methanol (250 mL) was added sodium borohydride (10.024 g, 264.984 mmol) slowly at 0° C. The resultant reaction mixture was stirred at RT for 16 hours. After completion, the reaction mixture was concentrated under reduced pressure and slowly poured into crushed ice. The solid precipitate that formed was filtered off and dried under reduced pressure. The crude thus obtained was purified by silica gel column chromatography using 0-5% MeOH in DCM to afford 5-hydroxymethyl-1-(4-methoxy-benzyl)-1H-benzo[cd]indol-2-one (5) (22 g, 78% Yield) as a yellow solid. ¹H NMR (d₆-DMSO, 400 MHz) δ 8.05 (d, J=7.2 Hz, 1H), 7.82 (d, J=7.12 Hz, 1H), 7.70 (d, J=8.48 Hz, 1H), 7.47 (t, J=7.84 Hz, 1H), 7.30 (d, J=8.48 Hz, 2H), 7.09 (d, J=7.12 Hz, 1H), 6.87 (d, J=8.56 Hz, 2H), 5.53 (t, J=5.52 Hz, 1H), 5.05-5.02 (m, 4H), 3.69 (s, 3H); LC MS: ES+319.8

Step 5: To a stirred suspension of 5-hydroxymethyl-1-(4-methoxy-benzyl)-1H-benzo[cd]indol-2-one (5) (22 g, 68.966 mmol) in DCM (350 mL), Et₃N (28.837 mL, 206.897 mmol) and methanesulfonyl chloride (206.897 mmol, 16.015 mL) were added at 0° C. and the resulting reaction mixture was stirred at RT for 16 h. After completion, the reaction mixture was diluted with ethyl acetate and washed with water, saturated aqueous sodium bicarbonate solution and brine, then dried over anhydrous sodium sulfate and concentrated under reduced pressure to afford 5-chloromethyl-1-(4-methoxy-benzyl)-1H-benzo[cd]indol-2-one, Compound 58, (19 g, 81.56% yield) as a yellow solid. ¹H NMR (d₆-DMSO, 400 MHZ) δ 8.07 (d, J=7.12 Hz, 1H), 7.90 (d, J=7.16 Hz, 1H), 7.80 (d, J=8.6 Hz, 1H), 7.55 (t, J=7.88 Hz, 1H), 7.31 (d, J=8.6 Hz, 2H), 7.13 (d, J=7.16 Hz, 1H), 6.87 (d, J=8.6 Hz, 2H), 5.30 (s, 2H), 5.03 (s, 2H), 3.69 (s, 3H);

### Example 45. Synthesis of 1-(2,6-dioxo-3-piperidyl)-2-oxo-benzo[cd]indole-5-carbaldehyde (Compound 69)

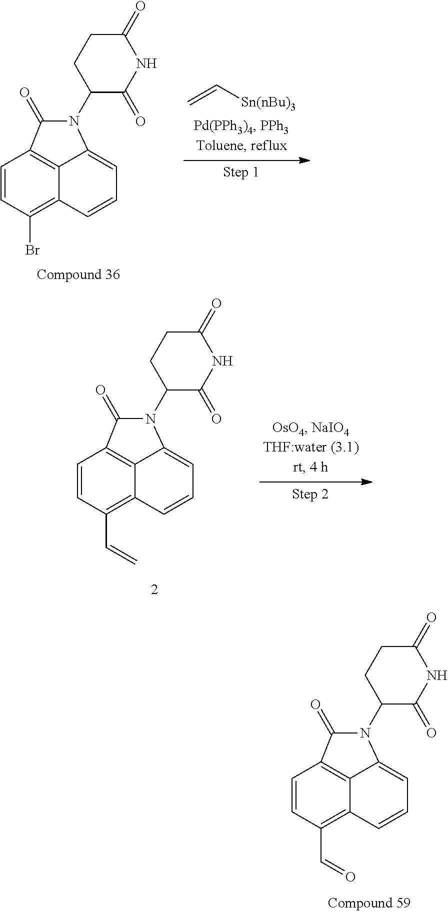

Step 1: To a stirred solution of 3-(5-bromo-2-oxo-benzo[cd]indol-1-yl)piperidine-2,6-dione, Compound 36, (20 g, 55.68 mmol) in toluene (500 mL) was purged argon for 20 min. Then tributyl vinyl tin (22.95 g, 72.39 mmol, 21.06 mL), triphenylphosphine (730.26 mg, 2.78 mmol) and tetrakis(triphenylphosphine)palladium (3.22 g, 2.78 mmol) were added and the reaction mixture was heated to 110° C. for 16 hours. After completion of the reaction, the solvent was evaporated under reduced pressure and the crude thus obtained was purified by silica gel column chromatography using 0-10% MeOH in DCM to afford 3-(2-oxo-5-vinyl-benzo[cd]indol-1-yl)piperidine-2,6-dione (2) (14.3 g, 59% yield) as a yellow solid. LC MS: ES+307.2

Step 2: To a stirred solution of 3-(2-oxo-5-vinyl-benzo[cd]indol-1-yl)piperidine-2,6-dione (2) (14 g, 45.70 mmol) in water (12 mL) and THE (36 mL) was added 4% solution of osmium tetroxide in water (572 mg, 507.35 μmol, 2 mL) and the reaction mixture was stirred at room temperature for 20 minutes followed by the addition of sodium periodate (24.44 g, 114.26 mmol). The resultant reaction mixture was then stirred at room temperature for 4 hours. After completion of the reaction, the reaction mixture was filtered through a pad of celite, washed with THE and 20% 2-propanol in DCM. The filtrate collected was then dried over anhydrous sodium sulfate and concentrated under reduced pressure. Crude thus obtained was purified by silica gel column chromatography 0-5% MeOH in DCM to afford 1-(2,6-dioxo-3-piperidyl)-2-oxo-benzo[cd]indole-5-carbaldehyde, Compound 59, (8 g, 37% yield) as a yellow solid. ¹H NMR (d₆-DMSO, 400 MHz) δ 11.16 (s, 1H), 10.52 (s, 1H), 8.46-8.43 (m, 2H), 8.31-8.30 (m, 1H), 7.71-7.67 (m, 1H), 7.27-7.25 (m, 1H), 5.48 (dd, J=12.48, 4.84 Hz, 1H), 2.95-2.90 (m, 1H), 2.79-2.74 (m, 1H), 2.68-2.63 (m, 1H), 2.13-2.08 (m, 1H); LC MS: ES+309.0.

General procedure A for elaboration of R¹ or R²:

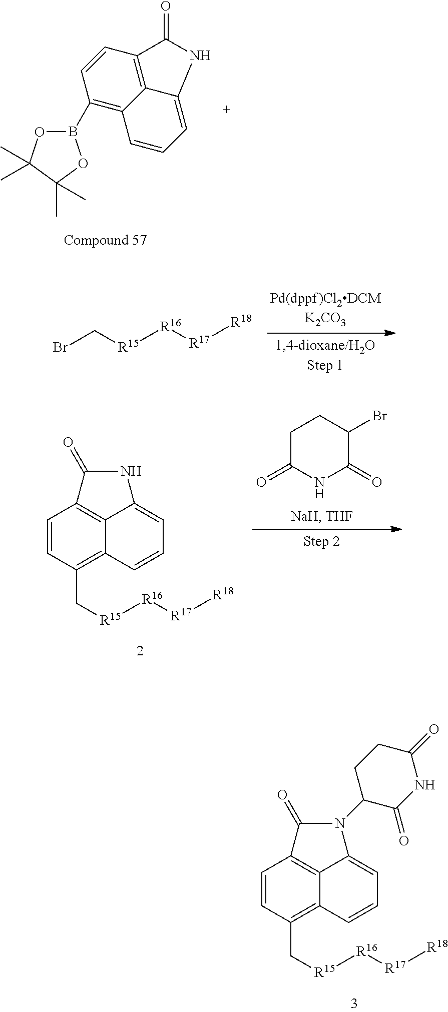

Step 1: To a stirred solution of Compound 57 (1.2 eq) in 1,4-dioxane:water (4:1, v/v, 0.14 M) was added akyl halides (1 eq) followed by K₂CO₃ (2 eq). The resultant reaction mixture was degassed with argon for 15 minutes. Subsequently, Pd(dppf)Cl₂ (0.1 eq) was added and the reaction mixture was heated at 90° C. for 12 hours. After completion of the reaction, the reaction mixture was cooled to RT and filtered through a pad of celite eluting with EtOAc. The combined filtrate was washed twice with cold water and dried over anhydrous sodium sulfate before being concentrated to a crude residue. The crude residue was purified by flash chromatography using 0 to 50% EtOAc in DCM as an eluent to afford the product 2.

Step 3: To a cooled solution (0° C.) of 2 (1 eq) in THE (0.02 M) was added NaH (60% oil dispersion, 10 eq) portionwise while maintaining a temperature <5° C. Upon complete addition, the mixture was stirred at room temperature for a further 15 minutes before the mixture was again cooled to 0° C. and 3-bromo-piperidine-2,6-dione (5 eq) was added before the mixture was heated to 70° C. for 1 hr. Upon reaction completion the mixture was cooled to 0° C. and quenched with ice water. The mixture was then extracted three times with EtOAc. The combined organic layers were separated, dried over anhydrous sodium sulfate and concentrated to a crude residue which was purified via RP-HPLC to afford the product 3.

General procedure B for elaboration of R¹ or R²:

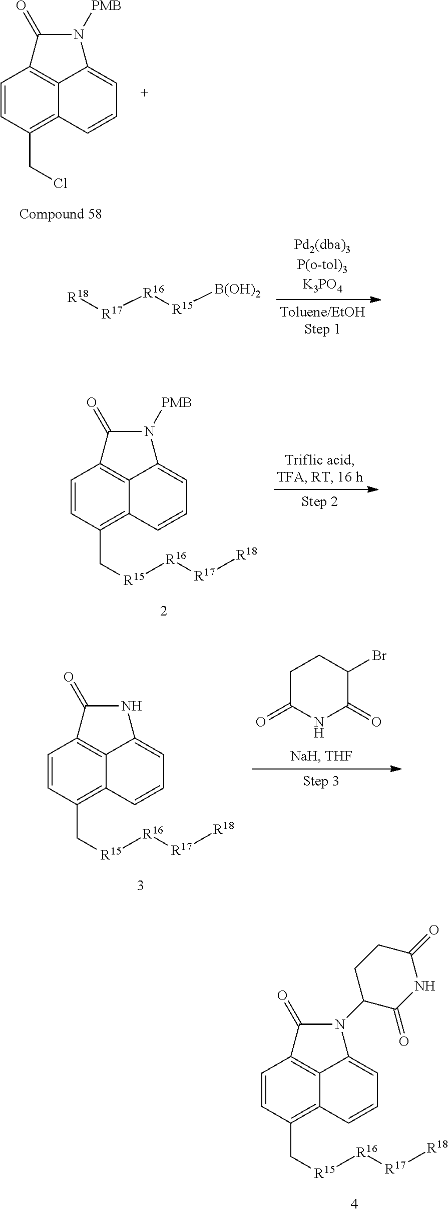

Step 1: To a degassed, argon-sparged, and stirred solution of Compound 58 (1 eq) and the corresponding boronic acid (1.2 eq) in toluene/EtOH (2:1, 0.1 M) in a pierced vial was added K₃PO₄ (2 eq), P(o-tol)₃ (0.2 eq) and Pd₂(dba)₃ (0.1 eq) before the mixture was heated at 100° C. for 16 hr. Reaction monitoring was performed using LC-MS. Upon reaction completion the mixture was cooled to RT and filtered through a pad of celite. The filtrate was then concentrated to dryness before being purified via silica gel column chromatography eluting with EtOAC in Hexaness 0-100% to afford the products.

Step 2: Intermediate 2 (1 eq) was suspended in TFA (0.2 M) at 0° C. before triflic acid (10 eq) was added dropwise while maintaining 0° C. The mixture was then allowed to stir at ambient temperature for 16 h. Upon reaction completion the mixture was concentrated to dryness under reduced pressure and the crude residue was basified with saturated aqueous sodium bicarbonate solution before extracting three times with EtOAC and washing with brine. The combined organic layers were then dried over anhydrous sodium sulfate before being filtered and concentrated to a crude residue. The crude residue was then purified via silica gel column chromatography eluting with EtOAC in DCM (20% to 60%) to afford the product 2.

Step 3: To a cooled solution (0° C.) of 2 (1 eq) in THE (0.02 M) was added NaH (60% oil dispersion, 10 eq) portionwise while maintaining a temperature <5° C. Upon complete addition, the mixture was stirred at room temperature for a further 15 minutes before the mixture was again cooled to 0° C. and 3-bromo-piperidine-2,6-dione (5 eq) was added before the mixture was heated to 70° C. for 1 hr. Upon reaction completion the mixture was cooled to 0° C. and quenched with ice water. The mixture was then extracted three times with EtOAc. The combined organic layers were separated, dried over anhydrous sodium sulfate and concentrated to a crude residue which was purified via RP-HPLC to afford the product 3.

General procedure C for elaboration of R¹ or R²:

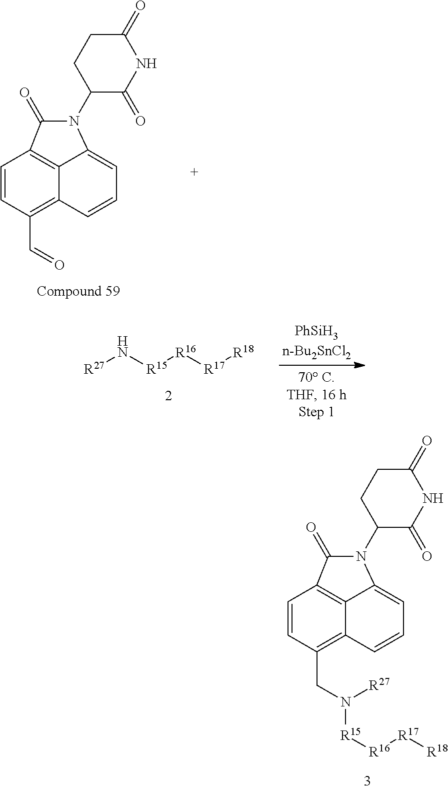

Step 1: To a stirred solution of Compound 59 (1 eq) in THF (0.15 M) was added the corresponding amine 2 (1 eq) followed by the addition of phenylsilane (1 eq) and dibutyltin dichloride (1.2 eq). The reaction mixture was then heated to 70° C. for 16 h with monitoring by LC-MS. Upon reaction completion the mixture was cooled to room temperature and immediately concentrated to dryness before being purified via RP-HPLC to afford the product 3.

### Example 46. Synthesis of 3-(5-(4-ethoxy-2-fluorobenzyl)-2-oxobenzo[cd]indol-1(2H)-yl)piperidine-2,6-dione (Compound 60)

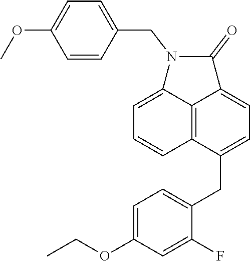 [SMILES: CCOc1ccc(Cc2ccc3c4c(cccc24)N(Cc2ccc(OC)cc2)C3=O)c(F)c1]

Step 1: 5-(4-ethoxy-2-fluorobenzyl)-1-(4-methoxybenzyl)benzo[cd]indol-2(1H)-one was obtained from general procedure B step 1 to afford a solid (260 mg, 588 μmol, 79% yield) LCMS (ESI): m/z 442.0 [M+H]⁺.

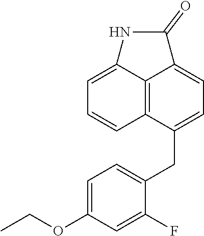 [SMILES: CCOc1ccc(Cc2ccc3c4c(cccc24)NC3=O)c(F)c1]

Step 2: 5-(4-ethoxy-2-fluorobenzyl)benzo[cd]indol-2(1H)-one was obtained from general procedure B step 2 to afford a solid (135 mg, 420 μmol, 71% yield) LCMS (ESI): m/z 332.0 [M+H]⁺.

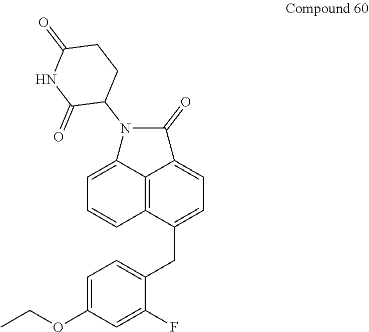 [SMILES: CCOc1ccc(Cc2ccc3c4c(cccc24)N(C2CCC(=O)NC2=O)C3=O)c(F)c1]

Step 3: 3-(5-(4-ethoxy-2-fluorobenzyl)-2-oxobenzo[cd]indol-1(2H)-yl)piperidine-2,6-dione, Compound 60, was obtained from general procedure B step 3 to afford a solid (6.4 mg, 29 μmol, 7% yield) LCMS (ESI): m/z 443.4 [M+H]⁺.

### Example 47. Synthesis of ethyl 4-((1-(2,6-dioxopiperidin-3-yl)-2-oxo-1,2-dihydrobenzo[cd]indol-5-yl)methyl)benzoate (Compound 61)

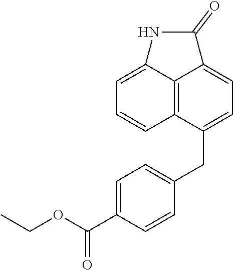 [SMILES: CCOC(=O)c1ccc(Cc2ccc3c4c(cccc24)NC3=O)cc1]

Step 1: ethyl 4-((2-oxo-1,2-dihydrobenzo[cd]indol-5-yl)methyl)benzoate was obtained from general procedure A step 1 to afford a solid. (40 mg, 120 μmol, 15% yield) LCMS (ESI): m/z 331.1 [M+H]⁺.

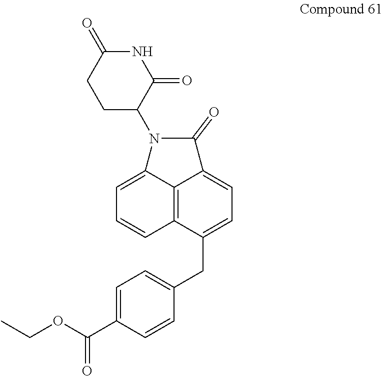 [SMILES: CCOC(=O)c1ccc(Cc2ccc3c4c(cccc24)N(C2CCC(=O)NC2=O)C3=O)cc1]

Step 2: ethyl 4-((1-(2,6-dioxopiperidin-3-yl)-2-oxo-1,2-dihydrobenzo[cd]indol-5-yl)methyl)benzoate, Compound 61, was obtained from general procedure A step 2 to afford a solid. (9 mg, 20 μmol, 17% yield) LCMS (ESI): m/z 442.3 [M+H]⁺.

### Example 48. Synthesis of ethyl 3-((1-(2,6-dioxopiperidin-3-yl)-2-oxo-1,2-dihydrobenzo[cd]indol-5-yl)methyl)benzoate (Compound 62)

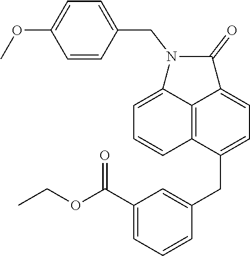 [SMILES: CCOC(=O)c1cccc(Cc2ccc3c4c(cccc24)N(Cc2ccc(OC)cc2)C3=O)c1]

Step 1: ethyl 3-((1-(4-methoxybenzyl)-2-oxo-1,2-dihydrobenzo[cd]indol-5-yl)methyl)benzoate was obtained from general procedure B step 1 to afford a solid. (220 mg, 487 μmol, 66% yield) LCMS (ESI): m/z 452.4 [M+H]⁺.

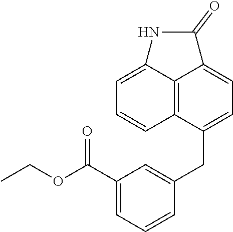 [SMILES: CCOC(=O)c1cccc(Cc2ccc3c4c(cccc24)NC3=O)c1]

Step 2: ethyl 3-((2-oxo-1,2-dihydrobenzo[cd]indol-5-yl)methyl)benzoate was obtained from general procedure B step 2 to afford a solid. (115 mg, 347 μmol, 71% yield) LCMS (ESI): m/z 332.0 [M+H]⁺.

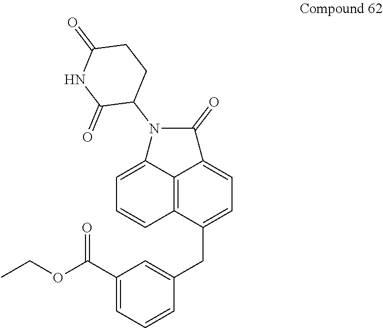 [SMILES: CCOC(=O)c1cccc(Cc2ccc3c4c(cccc24)N(C2CCC(=O)NC2=O)C3=O)c1]

Step 3: ethyl 3-((1-(2,6-dioxopiperidin-3-yl)-2-oxo-1,2-dihydrobenzo[cd]indol-5-yl)methyl)benzoate Compound 62 was obtained from general procedure B step 3 to afford a solid. (23 mg, 84 μmol, 56% yield) LCMS (ESI): m/z 443.2 [M+H]⁺.

### Example 49. Synthesis of 3-(2-oxo-5-((3-phenoxyazetidin-1-yl)methyl)benzo[cd]indol-1(2H)-yl)piperidine-2,6-dione (Compound 63)

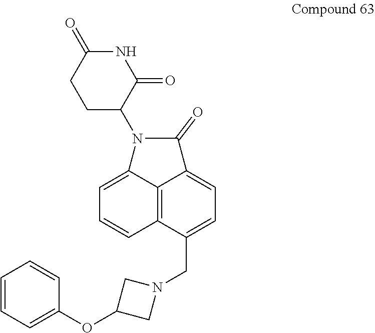 [SMILES: O=C1CCC(N2C(=O)c3ccc(CN4CC(Oc5ccccc5)C4)c4cccc2c34)C(=O)N1]

Step 1: 3-(2-oxo-5-((3-phenoxyazetidin-1-yl)methyl)benzo[cd]indol-1(2H)-yl)piperidine-2,6-dione Compound 63 was obtained from general procedure C to afford a solid. (8 mg, 18 μmol, 6% yield) LCMS (ESI): m/z 442.4 [M+H]⁺.

### Example 50. Synthesis of 2-(4-chlorophenyl)-N-[[1-(2,6-dioxo-3-piperidyl)-2-oxo-benzo[cd]indol-5-yl]methyl]-2,2-difluoro-acetamide (Compound 64)

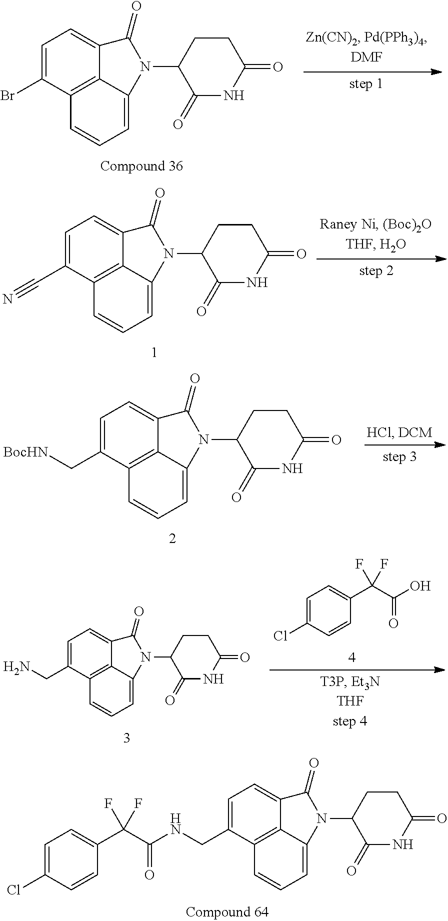 [SMILES: O=C1CCC(N2C(=O)c3ccc(Br)c4cccc2c34)C(=O)N1 ; N#Cc1ccc2c3c(cccc13)N(C1CCC(=O)NC1=O)C2=O ; CC(C)(C)OC(=O)NCc1ccc2c3c(cccc13)N(C1CCC(=O)NC1=O)C2=O ; NCc1ccc2c3c(cccc13)N(C1CCC(=O)NC1=O)C2=O ; O=C(O)C(F)(F)c1ccc(Cl)cc1 ; O=C1CCC(N2C(=O)c3ccc(CNC(=O)C(F)(F)c4ccc(Cl)cc4)c4cccc2c34)C(=O)N1]

Step 1: To a stirred solution of 3-(5-bromo-2-oxo-1H-acenaphthylen-1-yl)piperidine-2,6-dione Compound 36 (5.4 g, 15.08 mmol) in DMF (10 mL) in a degassed sealed tube was added zinc cyanide (1.77 g, 15.08 mmol, 956.92 μL). After degassing again for 5 minutes, tetrakis(triphenylphosphine)palladium (17.42 g, 15.08 mmol) was added and the solution was again degassed for 5 mins. After degassing, the sealed tube was closed and stirred at 90° C. for 5 hr. The progress of the reaction was monitored by TLC and LCMS. After reaction completion, the solution was diluted with ethyl acetate (30 mL), washed with water (30 mL) and then washed with brine (30 mL). The organic layer was separated, dried over anhydrous sodium sulfate, filtered, and concentrated under reduced pressure to provide the crude compound, which was then purified by silica gel column chromatography with 10 to 100% ethyl acetate in hexanes eluent to provide 1-(2,6-dioxo-3-piperidyl)-2-oxo-benzo[cd]indole-5-carbonitrile 1 (3.4 g, 9.68 mmol, 64% yield) as a yellow solid. LCMS (ESI): m/z 306.23 [M+H]⁺

Step 2: In an autoclave, a solution of 1-(2,6-dioxo-3-piperidyl)-2-oxo-benzo[cd]indole-5-carbonitrile 1 (5.5 g, 18.02 mmol) in THE (200 mL) was added tert-butoxycarbonyl tert-butyl carbonate (19.66 g, 90.08 mmol, 20.67 mL) followed by Raney Nickel 2800, slurry in H₂O, active catalyst (15.43 g, 180.16 mmol) at RT and the reaction mixture was stirred at RT under hydrogen atmosphere (400 psi) for 72 hr. The progress of the reaction was monitored by LCMS. After reaction completion, the reaction mixture was filtered through a pad of celite and the pad was washed twice with ethyl acetate (200 mL) carefully and all collected solvent was concentrated under reduced pressure to provide the crude compound. The crude residue was triturated with diethyl ether and pentane to provide tert-butyl N-[[1-(2,6-dioxo-3-piperidyl)-2-oxo-benzo[cd]indol-5-yl]methyl]carbamate 2 (3 g, 7.33 mmol, 40.67% yield) as a light yellow solid.

Step 3: An oven dried 50 mL single-necked round-bottomed flask was charged with tert-butyl N-[[1-(2,6-dioxo-3-piperidyl)-2-oxo-benzo[cd]indol-5-yl]methyl]carbamate 2 (600 mg, 1.47 mmol) in DCM (10 mL) and cooled to 0° C. To this solution was added hydrogen chloride solution 4.0M in dioxane (4.80 g, 131.65 mmol, 6 mL). The resulting mixture was stirred at room temperature for 1 hr. The progress of the reaction was monitored by UPLC analysis. Upon completion, the reaction mixture was concentrated under reduced pressure. The resulting crude product was washed with diethyl ether (20 mL) to afford 3-[5-(aminomethyl)-2-oxo-benzo[cd]indol-1-yl]piperidine-2,6-dione 3 (505 mg, 1.40 mmol, 95% yield) as a pale yellow solid. LCMS (ESI): m/z 310.2 [M+H]⁺.

Step 4: To a stirred solution of 3-[5-(aminomethyl)-2-oxo-benzo[cd]indol-1-yl]piperidine-2,6-dione 3 (0.040 g, 115.68 μmol) in THE (5 mL) was added 2-(4-chlorophenyl)-2,2-difluoro-acetic acid 4 (23.90 mg, 115.68 μmol) under an argon atmosphere. The reaction mixture was cooled to 0° C., then triethylamine (58.53 mg, 578.40 μmol, 80.62 μL) and propylphosphonic anhydride solution (184.04 mg, 289.20 μmol, 172.00 μL, 50% purity) were sequentially added and the reaction mixture was stirred at RT for 16 hr. The progress of the reaction was monitored by TLC, and after reaction completion the reaction mixture was diluted with ethyl acetate, washed with sodium bicarbonate solution, and washed with brine. The combined organic layers were separated, dried over anhydrous sodium sulfate, filtered and concentrated under reduced pressure to provide the crude compound. The crude compound was purified by silica gel column chromatography eluted with 1 to 5% MeOH in DCM to provide 2-(4-chlorophenyl)-N-[[1-(2,6-dioxo-3-piperidyl)-2-oxo-benzo[cd]indol-5-yl]methyl]-2,2-difluoro-acetamide Compound 64 (20 mg, 39.21 μmol, 33% yield) as a yellow solid. ¹HNMR (400 MHz, DMSO-d₆): δ 11.12 (s, 1H), 9.79-9.78 (m, 1H), 8.06-8.04 (d, J=8 Hz, 1H), 7.76-7.74 (d, J=8 Hz, 1H), 7.65-7.63 (d, J=8 Hz, 1H), 7.61 (s, 4H), 7.53-7.49 (m, 1H), 7.17-7.15 (d, J=8 Hz, 1H), 5.46-5.42 (m, 1H), 4.89-4.88 (d, J=4 Hz, 2H), 2.94 (m, 1H), 2.76-2.73 (m, 1H), 2.66-2.63 (m, 1H), 2.10-2.09 (m, 1H). LC-MS:(ES+)=498.2 [M+H]⁺

### Example 51. Synthesis of 3-[2-oxo-5-[[4-(trifluoromethyl)phenyl]methyl]benzo[cd]indol-1-yl]piperidine-2,6-dione (Compound 65)

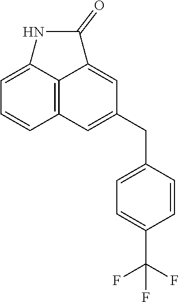 [SMILES: O=C1Nc2cccc3cc(Cc4ccc(C(F)(F)F)cc4)cc1c23]

Step 1: 4-[[4-(trifluoromethyl)phenyl]methyl]-1H-benzo[cd]indol-2-one was obtained from general procedure A step 1 by substituting Compound 57 for 4-(4,4,5,5-tetramethyl-1,3,2-dioxaborolan-2-yl)-1H-benzo[cd]indol-2-one to afford a solid. (90 mg, 124 μmol, 12% yield). LC-MS (ES+): m/z 328.2 [M+H]⁺.

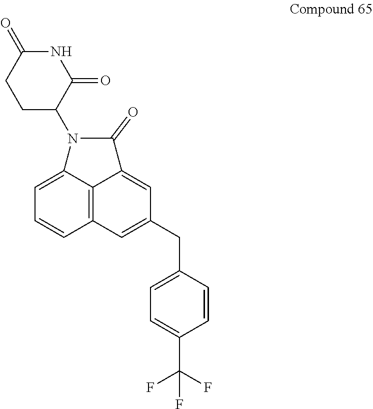 [SMILES: O=C1CCC(N2C(=O)c3cc(Cc4ccc(C(F)(F)F)cc4)cc4cccc2c34)C(=O)N1]

Step 2: 3-[2-oxo-4-[[4-(trifluoromethyl)phenyl]methyl]benzo[cd]indol-1-yl]piperidine-2,6-dione, Compound 65, was obtained from general procedure A step 2 to afford a solid. (20 mg, 45 μmol, 13% yield). LC-MS (ES⁻): m/z 436.9 [M−H]⁺.

### Example 52. Synthesis of 4-[[1-(2,6-dioxo-3-piperidyl)-2-oxo-benzo[cd]indol-5-yl]methyl]benzonitrile (Compound 66)

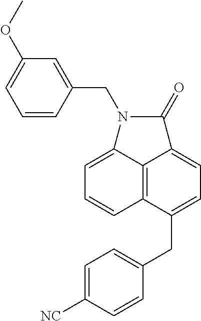 [SMILES: COc1cccc(CN2C(=O)c3ccc(Cc4ccc(C#N)cc4)c4cccc2c34)c1]

Step 1: 4-[[1-[(4-methoxyphenyl)methyl]-2-oxo-benzo[cd]indol-5-yl]methyl]benzonitrile was obtained from general procedure B step 1 to afford a solid (220 mg, 534 μmol, 73% yield). LC-MS (ES+): m/z 405.4 [M+H]⁺.

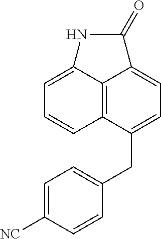 [SMILES: N#Cc1ccc(Cc2ccc3c4c(cccc24)NC3=O)cc1]

Step 2: 4-[(2-oxo-1H-benzo[cd]indol-5-yl)methyl]benzonitrile was obtained from general procedure B step 2 to afford a solid (64 mg, 135 μmol, 25% yield). LC-MS (ES+): m/z 285.1 [M+H]⁺.

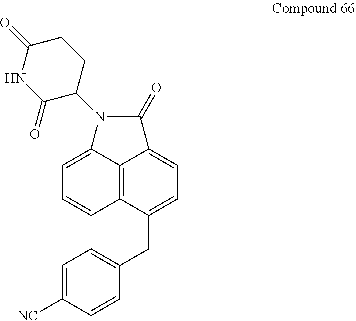 [SMILES: N#Cc1ccc(Cc2ccc3c4c(cccc24)N(C2CCC(=O)NC2=O)C3=O)cc1]

Step 3: 4-[[1-(2,6-dioxo-3-piperidyl)-2-oxo-benzo[cd]indol-5-yl]methyl]benzonitrile, Compound 66, was obtained from general procedure B step 3 to afford a solid (8 mg, 20 μmol, 9% yield). LC-MS (ES+): m/z 394.4 [M−H]⁺.

### Example 53. Synthesis of 3-[5-[(3-chloro-4-fluoro-phenyl)methyl]-2-oxo-benzo[cd]indol-1-yl]piperidine-2,6-dione (Compound 67)

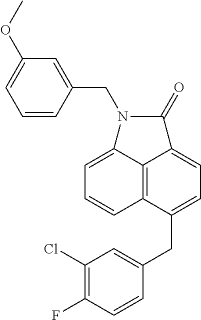 [SMILES: COc1cccc(CN2C(=O)c3ccc(Cc4ccc(F)c(Cl)c4)c4cccc2c34)c1]

Step 1: 5-[(3-chloro-4-fluoro-phenyl)methyl]-1H-benzo[cd]indol-2-one was obtained from general procedure B step 1 to afford a solid (230 mg, 532 μmol, 72% yield). LC-MS (ES+): m/z 424.3 [M+H]⁺.

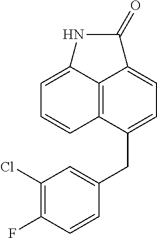 [SMILES: O=C1Nc2cccc3c(Cc4ccc(F)c(Cl)c4)ccc1c23]

Step 2: 5-[(4-ethoxyphenyl)methyl]-1H-benzo[cd]indol-2-one was obtained from general procedure B step 2 to afford a solid (140 mg, 166 μmol, 76% yield). LC-MS (ES+): m/z 312.2 [M+H]⁺.

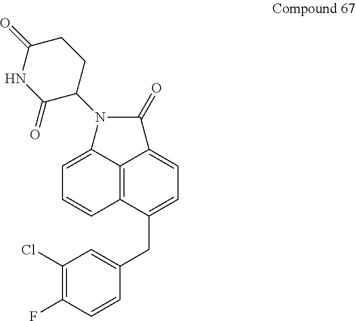 [SMILES: O=C1CCC(N2C(=O)c3ccc(Cc4ccc(F)c(Cl)c4)c4cccc2c34)C(=O)N1]

Step 3: 3-[5-[(3-chloro-4-fluoro-phenyl)methyl]-2-oxo-benzo[cd]indol-1-yl]piperidine-2,6-dione, Compound 67, was obtained from general procedure B step 3 to afford a solid (57 mg, 134 μmol, 30% yield). LC-MS (ES⁻): m/z 421.3 [M−H]⁺.

### Example 54. Synthesis of 3-[5-[(4-ethoxyphenyl)methyl]-2-oxo-benzo[cd]indol-1-yl]piperidine-2,6-dione (Compound 68)

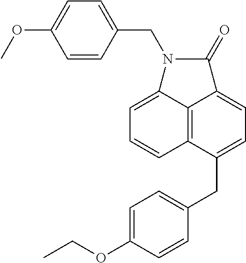 [SMILES: CCOc1ccc(Cc2ccc3c4c(cccc24)N(Cc2ccc(OC)cc2)C3=O)cc1]

Step 1: 5-[(4-ethoxyphenyl)methyl]-1-[(4-methoxyphenyl)methyl]benzo[cd]indol-2-one was obtained from general procedure B step 1 to afford a solid (170 mg, 401 μmol, 54% yield). LC-MS (ES+): m/z 424.3 [M+H]⁺.

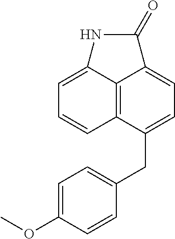 [SMILES: COc1ccc(Cc2ccc3c4c(cccc24)NC3=O)cc1]

Step 2: 5-[(4-ethoxyphenyl)methyl]-1H-benzo[cd]indol-2-one was obtained from general procedure B step 2 to afford a solid (65 mg, 90 μmol, 24% yield). LC-MS (ES+): m/z 304.1 [M+H]⁺.

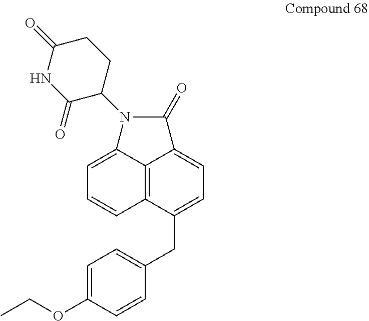 [SMILES: CCOc1ccc(Cc2ccc3c4c(cccc24)N(C2CCC(=O)NC2=O)C3=O)cc1]

Step 3: 3-[5-[(4-ethoxyphenyl)methyl]-2-oxo-benzo[cd]indol-1-yl]piperidine-2,6-dione, Compound 68, was obtained from general procedure B step 3 to afford a solid (6 mg, 14 μmol, 29% yield). LC-MS (ES⁻): m/z 413.4 [M−H]⁺.

### Example 55. Synthesis of ethyl 5-[[1-(2,6-dioxo-3-piperidyl)-2-oxo-benzo[cd]indol-5-yl]methyl]-2-fluoro-benzoate (Compound 69)

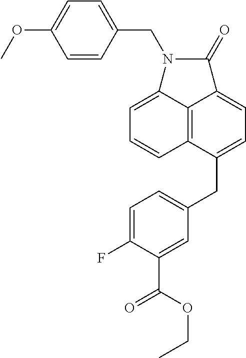 [SMILES: CCOC(=O)c1cc(Cc2ccc3c4c(cccc24)N(Cc2ccc(OC)cc2)C3=O)ccc1F]

Step 1: ethyl 2-fluoro-5-[[1-[(4-methoxyphenyl)methyl]-2-oxo-benzo[cd]indol-5-yl]methyl]benzoate (260 mg, 588 μmol, 66%% yield) was obtained from general procedure B step 1. LC-MS (ES⁺): m/z 469.4 [M+H]⁺.

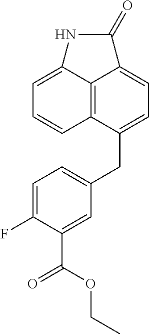 [SMILES: CCOC(=O)c1cc(Cc2ccc3c4c(cccc24)NC3=O)ccc1F]

Step 2: ethyl 2-fluoro-5-((2-oxo-1,2-dihydrobenzo[cd]indol-5-yl)methyl)benzoate (145 mg, 76% yield) was obtained from general procedure B step 2. LC-MS (ES⁺): m/z 350.3 [M+H]⁺.

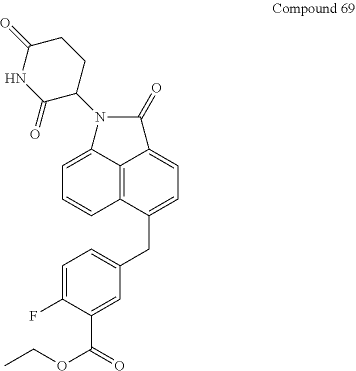 [SMILES: CCOC(=O)c1cc(Cc2ccc3c4c(cccc24)N(C2CCC(=O)NC2=O)C3=O)ccc1F]

Step 3: ethyl 5-[[1-(2,6-dioxo-3-piperidyl)-2-oxo-benzo[cd]indol-5-yl]methyl]-2-fluoro-benzoate Compound 69 (8 mg, 17 μmol, 4% yield) was obtained from general procedure B step 3 as a solid. LC-MS (ES⁺): m/z 460.1 [M+H]⁺.

### Example 56. Synthesis of tert-butyl 4-[4-[1-(2,6-dioxo-3-piperidyl)-2-oxo-benzo[cd]indole-6-carbonyl]pyrazol-1-yl]-4-methyl-piperidine-1-carboxylate (Compound 70))

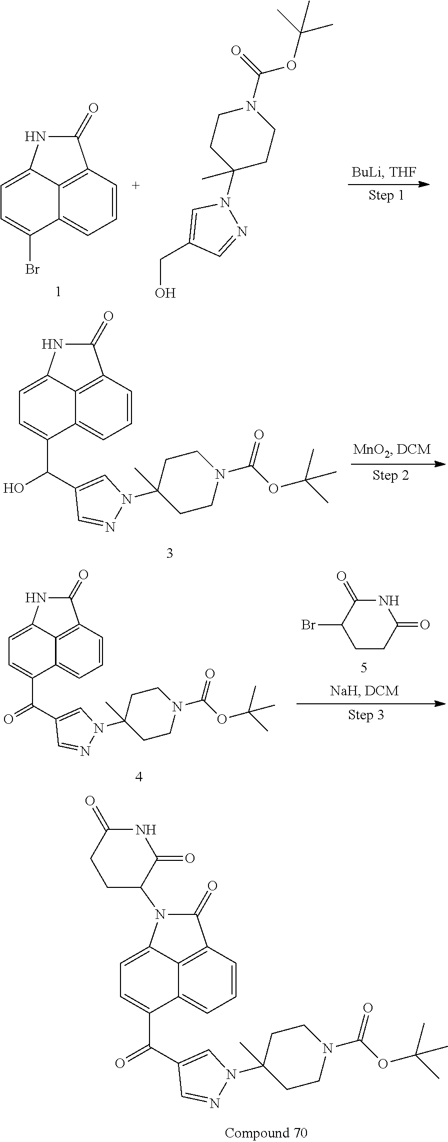 [SMILES: O=C1Nc2ccc(Br)c3cccc1c23 ; CC(C)(C)OC(=O)N1CCC(C)(n2cc(CO)cn2)CC1 ; CC(C)(C)OC(=O)N1CCC(C)(n2cc(C(O)c3ccc4c5c(cccc35)C(=O)N4)cn2)CC1 ; CC(C)(C)OC(=O)N1CCC(C)(n2cc(C(=O)c3ccc4c5c(cccc35)C(=O)N4)cn2)CC1 ; O=C1CCC(Br)C(=O)N1 ; CC(C)(C)OC(=O)N1CCC(C)(n2cc(C(=O)c3ccc4c5c(cccc35)C(=O)N4C3CCC(=O)NC3=O)cn2)CC1]

Step 1: To the stirred solution of 6-bromo-1H-benzo[cd]indol-2-one 1 (510 mg, 2.06 mmol) in THE (7 mL) was added butyllithium (2.15 M, 2.10 mL) at −78° C. and after the addition was complete the temperature was allowed to increase to −40° C. and the reaction mixture was stirred at the same temperature for 30 minutes followed by the addition of tert-butyl 4-(4-formylpyrazol-1-yl)-4-methyl-piperidine-1-carboxylate 2 (603.10 mg, 2.06 mmol) in THE (7 mL) at −78° C. and then the reaction mixture was allowed to warm to room temperature and was continued for 16 hours. TLC was checked which showed formation of the desired spot. The reaction mixture was quenched with ammonium chloride solution, diluted with ethyl acetate, washed with water and the organic fraction was separated. It was then dried over anhydrous sodium sulphate and evaporated under reduced pressure to obtain the crude compound which was purified by flash chromatography using 0-5% MeOH-DCM to afford tert-butyl 4-[4-[hydroxy-(2-oxo-1H-benzo[cd]indol-6-yl)methyl]pyrazol-1-yl]-4-methyl-piperidine-1-carboxylate 3 (210.0 mg, 426.32 μmol, 21% yield) as brown solid. LC-MS (ES+): m/z 463.2 [M+H]⁺.

Step 2: To the stirred solution of tert-butyl 4-[4-[hydroxy-(2-oxo-1H-benzo[cd]indol-6-yl)methyl]pyrazol-1-yl]-4-methyl-piperidine-1-carboxylate 3 (210.0 mg, 454.02 μmol) in DCM (4.0 mL) was added Manganese dioxide (394.71 mg, 4.54 mmol) and the reaction mixture was stirred at room temperature for 16 hours. The reaction mixture was filtered over celite bed, washed with ethyl acetate and the filtrate was evaporated under reduced pressure to obtain the crude compound which was purified by flash chromatography using 0-5% MeOH-DCM to afford tert-butyl 4-methyl-4-[4-(2-oxo-1H-benzo[cd]indole-6-carbonyl)pyrazol-1-yl]piperidine-1-carboxylate 4 (135.0 mg, 284.64 μmol, 63% yield) as a pale yellow solid. LC-MS (ES⁺): m/z 461.4 [M+H].

Step 3: To the stirred solution of tert-butyl 4-methyl-4-[4-(2-oxo-1H-benzo[cd]indole-6-carbonyl)pyrazol-1-yl]piperidine-1-carboxylate 4 (135.0 mg, 293.14 μmol) in DMF (1 mL) was added sodium hydride (60% dispersion in mineral oil) (29.31 mg, 732.86 μmol) while maintaining at cold temperature. The reaction mixture was heated at 70° C. for 1 hour followed by the addition of 3-bromopiperidine-2,6-dione 5 (56.29 mg, 293.14 μmol) and heated at 70° C. for 4 hours. This was followed by the another addition for 3-bromopiperidine-2,6-dione 5 (56.29 mg, 293.14 μmol) and the reaction was continued at 70° C. for 16 hours. The reaction mixture was diluted with ethyl acetate, washed with water and the organic fraction was separated. It was then dried over anhydrous sodium sulphate and evaporated under reduced pressure to obtain the crude which was purified by preparative TLC (40% ethyl acetate-DCM) to afford tert-butyl 4-[4-[1-(2,6-dioxo-3-piperidyl)-2-oxo-benzo[cd]indole-6-carbonyl]pyrazol-1-yl]-4-methyl-piperidine-1-carboxylate Compound 70 (10.0 mg, 17.38 μmol, 6% yield) as a light yellow solid. LC-MS (ES⁺): m/z 572.5 [M+H]⁺.

### Example 57. Synthesis of 3-(10-oxo-14,16,17-triazatricyclododeca-(2),1(7),8-trien-17-yl)piperidine-2,6-dione (Compound 71)

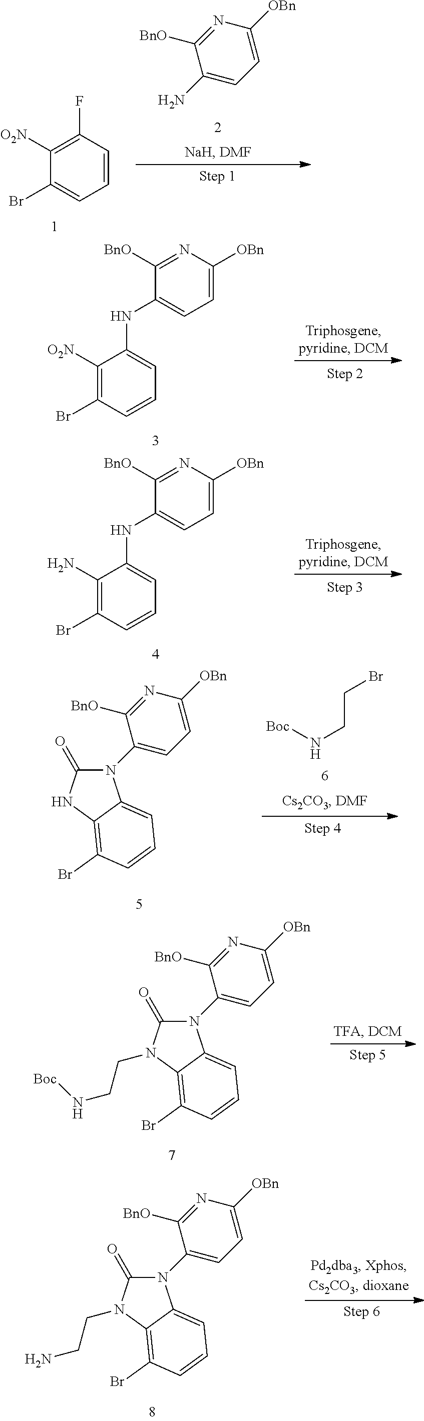

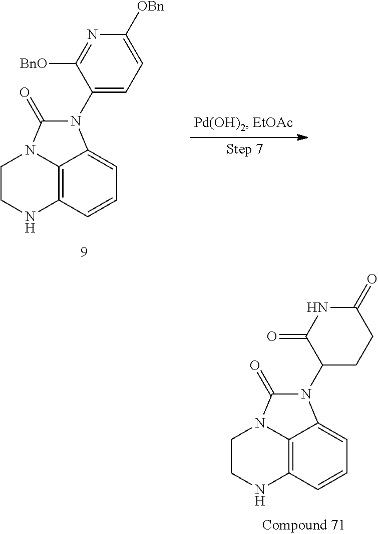 [SMILES: O=c1n2c3c(cccc3n1-c1ccc(OCc3ccccc3)nc1OCc1ccccc1)NCC2 ; O=C1CCC(n2c(=O)n3c4c(cccc42)NCC3)C(=O)N1]

Step 1: To a stirred solution of 2,6-dibenzyloxypyridin-3-amine 2 (500 mg, 1.63 mmol) in DMF (5 mL), sodium hydride (60% dispersion in mineral oil (71.80 mg, 1.80 mmol) and 1-bromo-3-fluoro-2-nitro-benzene 1 (430.86 mg, 1.96 mmol) were added at 0° C. The resulting reaction mixture was stirred at 60° C. for 16 hours and the progress of the reaction was monitored by UPLC. The reaction mixture was quenched with ice-water (10 mL), extracted with ethyl acetate (20 mL×2), then combined organic layer was dried over anhydrous sodium sulfate and concentrated under reduced pressure to get crude compound which was purified by column chromatography by using 100-200 mesh silica gel compound eluted with 5-10% ethyl acetate to afford 2,6-dibenzyloxy-N-(3-bromo-2-nitro-phenyl)pyridin-3-amine 3 (500 mg, 834.21 μmol, 51% yield) as a brown color gummy liquid. LC-MS (ES⁺): m/z 508.0 [M+H]⁺.

Step 2: To a stirred solution of 2,6-dibenzyloxy-N-(3-bromo-2-nitro-phenyl)pyridin-3-amine 3 (500 mg, 987.46 μmol) in methanol (10 mL) were added ammonia; hydrochloride (528.21 mg, 9.87 mmol) and zinc (645.70 mg, 9.87 mmol, 90.43 μL) at 26° C. The reaction mixture was stirred at 26° C. for 0.5 hour. The progress of reaction was monitored by TLC. After completion of the reaction as indicated by TLC, the reaction mixture was filtered through celite pad, filtrate was concentrated under reduced pressure to get crude compound. The crude product was purified by column chromatography using 50 g of silica gel (100-200 mesh) using a gradient of 0-100% Ethyl acetate—Hexanes with the desired product eluting at 20-30% ethyl acetate—hexanes. The resulting 3-bromo-N1-(2,6-dibenzyloxy-3-pyridyl)benzene-1,2-diamine 4 (380 mg, 726.39 μmol, 74% yield) as a white solid. LC-MS (ES⁺): m/z 478.2 [M+H]⁺.

Step 3: To a stirred solution of 3-bromo-N1-(2,6-dibenzyloxy-3-pyridyl)benzene-1,2-diamine 4 (380 mg, 797.71 μmol) in DCM (10 mL) were added pyridine (189.30 mg, 2.39 mmol, 193.55 μL) followed by bis(trichloromethyl) carbonate (236.72 mg, 797.71 μmol) at 0° C. The reaction mixture was stirred at 26° C. and stirred for 0.5 hour. Progress of reaction was monitored by TLC. After completion of the reaction, the reaction mixture was quenched with ice cold water (20 mL), extracted with DCM (2×30 mL). The combined organic layer was washed with brine solution (20 mL) and dried over anhydrous sodium sulfate, filtered, and concentrated under reduced pressure to obtained crude compound, which was purified by column chromatography using 10 g of silica gel (100-200 mesh) using a gradient of 0-100% Ethyl acetate—Hexanes with the desired product eluting at 35-40% Ethyl acetate/Hexanes. The resulting 7-bromo-3-(2,6-dibenzyloxy-3-pyridyl)-1H-benzimidazol-2-one 5 (250 mg, 410.96 μmol, 52% yield) as a pale-brown gummy solid. LC-MS (ES⁺): m/z 504.0 [M+2]⁺.

Step 4: To a stirred solution of 7-bromo-3-(2,6-dibenzyloxy-3-pyridyl)-1H-benzimidazol-2-one 5 (250 mg, 497.65 μmol) in DMF (3 mL) then dicesium carbonate (486.43 mg, 1.49 mmol) and tert-butyl N-(2-bromoethyl)carbamate 6 (223.04 mg, 995.31 μmol) were added at 26° C. the resulting reaction mixture was stirred at 26° C. for 16 hour and the progress of the reaction was monitored by LCMS. The reaction mixture was poured in ice water (25 mL), then obtained solid was filtered and dried under vacuum to get tert-butyl N-[2-[7-bromo-3-(2,6-dibenzyloxy-3-pyridyl)-2-oxo-benzimidazol-1-yl]ethyl]carbamate 7 (240 mg, 343.45 μmol, 69% yield) as a white solid. LC-MS (ES⁺): m/z 545.1 [M−Boc+H]⁺.

Step 5: To a stirred solution of tert-butyl N-[2-[7-bromo-3-(2,6-dibenzyloxy-3-pyridyl)-2-oxo-benzimidazol-1-yl]ethyl]carbamate 7 (500 mg, 774.54 μmol) in DCM (5 mL) under nitrogen condition. The was added Trifluoroacetic acid (353.26 mg, 3.10 mmol, 238.69 μL) at 0° C. The reaction mixture was stirred at 26° C. for 2 hr. Progress of reaction was monitored by TLC. After completion of the reaction as indicated by TLC, the reaction mixture was concentrated under reduced pressure to get 3-(2-aminoethyl)-4-bromo-1-(2,6-dibenzyloxy-3-pyridyl)benzimidazol-2-one 8 (500 mg, 657.59 μmol, 85% yield) as a colorless gummy liquid. LC-MS (ES⁺): m/z 545.2 [M+H]⁺.

Step 6: Into a 25 mL sealed-tube reactor containing a well-stirred solution of 3-(2-aminoethyl)-4-bromo-1-(2,6-dibenzyloxy-3-pyridyl)benzimidazol-2-one hydrochloride 8 (500 mg, 859.27 μmol) in 1,4 Dioxane (10 mL) was added dicesium carbonate (1.12 g, 3.44 mmol) at ambient temperature under nitrogen atmosphere and the resulting mixture was degassed by bubbling nitrogen gas into the reaction mixture for 10 minutes. Subsequently, (1E,4E)-1,5-diphenylpenta-1,4-dien-3-one palladium (157.37 mg, 171.85 μmol) and dicyclohexyl-[2-(2,4,6-triisopropylphenyl)phenyl]phosphane (81.93 mg, 171.85 μmol) were added to the reaction mixture and reaction mixture was heated to 100° C. for 16 hours. After completion of the reaction as indicated by TLC, the reaction mixture was cooled to room temperature and poured into water (20 mL) and extracted with EtOAc (2×20 mL). Organic phases were combined and washed with brine (10 mL). The combined organic phases were dried over anhydrous sodium sulfate, filtered and the filtrate was concentrated under reduced pressure to get a crude residue, which was purified by flash silica-gel (230-400 mesh) column with 0-100% EtOAc/pet ether while desired compound eluting at 80-100% to afford 31-(2,6-dibenzyloxy-3-pyridyl)-29,30,31-triazatricyclododeca-6(12),11(21),22(25)-trien-27-one 9 (150 mg, 319.69 μmol, 37% yield) as light brown color solid. LC-MS (ES⁺): m/z 465.0 [M+H]⁺.

Step 7: Into a 50 mL single-necked round-bottomed flask containing a well-stirred suspension of 31-(2,6-dibenzyloxy-3-pyridyl)-29,30,31-triazatricyclododeca-6(12),11(21),22(25)-trien-27-one 9 (140 mg, 301.39 μmol) in ethyl acetate (3 mL) was added Palladium hydroxide on carbon 10%, 50% wet (140.00 mg, 1.13 mmol) at ambient temperature under nitrogen atmosphere. The resulting suspension was stirred at ambient temperature under hydrogen atmosphere (bladder) for 16 hr. After complete consumption of the starting material as indicated by UPLC, the reaction mixture was filtered through a pad of celite and washed with 1:1 ratio of 2-propanol/DCM (200 mL). Combined filtrate was concentrated under reduced pressure to get a crude residue which was purified by prep HPLC following a method: Column: SELECT C18; 150*21.2 MM; 5 UM; Mobile Phase: 0.1% TFA IN H20:ACN; Flow rate: 15 mL\min; RT=8.0 min. to obtain 3-(10-oxo-14,16,17-triazatricyclododeca-(2),1(7),8-trien-17-yl)piperidine-2,6-dione Compound 71 (25 mg, 62.10 μmol, 21% yield) as an off-white solid. LC-MS (ES⁺): m/z 287.0 [M+H]⁺.

### Example 58. Synthesis of 3-(11-oxo-15,17,18-triazatricyclotrideca-,2(9),8(10)-trien-18-yl)piperidine-2,6-dione (Compound 72)

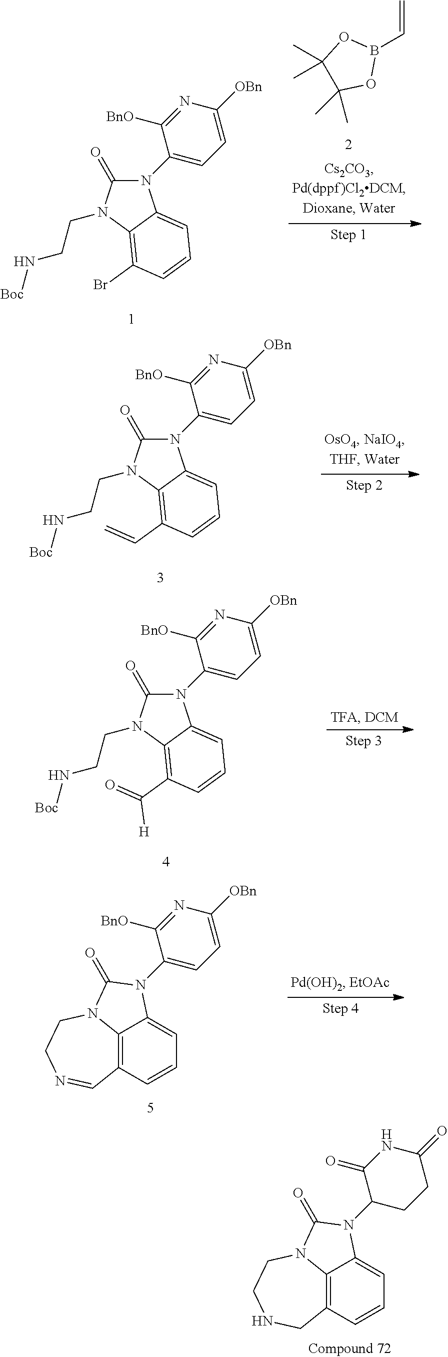 [SMILES: CC(C)(C)OC(=O)NCCn1c(=O)n(-c2ccc(OCc3ccccc3)nc2OCc2ccccc2)c2cccc(Br)c21 ; C=CB1OC(C)(C)C(C)(C)O1 ; C=Cc1cccc2c1n(CCNC(=O)OC(C)(C)C)c(=O)n2-c1ccc(OCc2ccccc2)nc1OCc1ccccc1 ; CC(C)(C)OC(=O)NCCn1c(=O)n(-c2ccc(OCc3ccccc3)nc2OCc2ccccc2)c2cccc(C=O)c21 ; O=c1n2c3c(cccc3n1-c1ccc(OCc3ccccc3)nc1OCc1ccccc1)C=NCC2 ; O=C1CCC(n2c(=O)n3c4c(cccc42)CNCC3)C(=O)N1]

Step 1: To a 25 mL sealed tube containing a well-stirred solution of a mixture of tert-butyl N-[2-[7-bromo-3-(2,6-dibenzyloxy-3-pyridyl)-2-oxo-benzimidazol-1-yl]ethyl]carbamate 1 (500 mg, 774.54 μmol) and 4,4,5,5-tetramethyl-2-vinyl-1,3,2-dioxaborolane 2 (238.58 mg, 1.55 mmol) in 1,4-dioxane (15 mL) and water (3 mL) was added cesium carbonate (757.09 mg, 2.32 mmol) at ambient temperature. The reaction mixture was degassed under nitrogen atmosphere for 10 minutes, added [1,1′-Bis(diphenylphosphino)ferrocene]dichloropalladium(II), complex with dichloromethane (63.25 mg, 77.45 μmol) and stirred at 100° C. for 12 hours. Progress of the reaction was monitored by TLC. After consumption of the starting material, the reaction mixture was allowed to attain room temperature and filtered through a celite pad, washed with ethyl acetate (2×20 mL). The filtrate was washed with water and dried over anhydrous sodium sulfate, filtered, and concentrated under reduced pressure. The crude product was purified by flash column chromatography (25 g silica gel 230-400 mesh, 0-100% ethyl acetate in hexane) to afford tert-butyl N-[2-[3-(2,6-dibenzyloxy-3-pyridyl)-2-oxo-7-vinyl-benzimidazol-1-yl]ethyl]carbamate 3 (410 mg, 678.00 μmol, 88% yield) obtained as a pale yellow solid. LC-MS (ES⁺): m/z 493.0 [M+H−Boc]⁺.

Step 2: To a stirred solution of tert-butyl N-[2-[3-(2,6-dibenzyloxy-3-pyridyl)-2-oxo-7-vinyl-benzimidazol-1-yl]ethyl]carbamate 3 (590 mg, 995.47 μmol) in THE (12 mL) and water (6 mL) was added sodium periodate (638.77 mg, 2.99 mmol) followed by Osmium(VIII) oxide, 4% aq. soln. (632.69 mg, 99.55 μmol, 632.69 μL) at 27° C., stirred for 1 hour at the same temperature. Progress of the reaction was monitored by TLC. After completion of the reaction as indicated by TLC, the reaction mixture was diluted with ethyl acetate (15 ml), washed with water (10 ml). The organic layer was dried over anhydrous sodium sulfate, filtered and concentrated under reduced pressure to give crude which was triturated with hexanes and dried under high vacuum to obtain tert-butyl N-[2-[3-(2,6-dibenzyloxy-3-pyridyl)-7-formyl-2-oxo-benzimidazol-1-yl]ethyl]carbamate 4 (430 mg, 615.36 μmol, 62% yield) as a pale yellow solid. LC-MS (ES⁺): m/z 495.3 [M+H]⁺.

Step 3: To a stirred solution of tert-butyl N-[2-[3-(2,6-dibenzyloxy-3-pyridyl)-7-formyl-2-oxo-benzimidazol-1-yl]ethyl]carbamate 4 (430 mg, 723.11 μmol) in DCM (3 mL), was added trifluoroacetic acid, 99% (247.35 mg, 2.17 mmol, 167.13 μL) at 0° C. and the reaction mixture was stirred at 27° C. for 3 hours. Progress of the reaction was monitored by LCMS, which indicated the formation of the desired product. The reaction mixture was concentrated under reduced pressure, the residue obtained was triturated with MTBE and dried under high vacuum to obtain 32-(2,6-dibenzyloxy-3-pyridyl)-30,31,32-triazatricyclotrideca-6(12),11(22),17(30),23(26)-tetraen-28-one trifluoroacetate 5 (425 mg, 509.81 μmol, 70.50% yield) as a pale brown solid. LC-MS (ES⁺): m/z 477.3 [M+H]⁺.

Step 4: To a stirred solution of 32-(2,6-dibenzyloxy-3-pyridyl)-30,31,32-triazatricyclotrideca-6(11),12(23),17(30),22(26)-tetraen-28-one trifluoroacetate 5 (420 mg, 497.84 μmol) in ethyl acetate (30 mL) was added palladium hydroxide on carbon, 20 wt. % 50% water (300 mg, 2.14 mmol) at 27° C. and the reaction mixture was allowed to stir for 48 hours under hydrogen atmosphere with bladder pressure. Progress of the reaction was monitored by LCMS. The reaction mixture was filtered through a celite pad, washed with 5% TFA in THE (2×50 ml). The filtrates were combined and concentrated under reduced pressure to give crude product, which was purified by prep HPLC and lyophilized to afford 3-(11-oxo-15,17,18-triazatricyclotrideca-,2(9),8(10)-trien-18-yl)piperidine-2,6-dione Compound 72 (13 mg, 31.14 μmol, 6% yield) as a white solid. LC-MS (ES⁺): m/z 301.0 [M+H]⁺.

### Example 59. Synthesis of 3-(5-(4-ethoxybenzyl)-2-oxobenzo[cd]indol-1(2H)-yl)piperidine-2,6-dione (Compound 74)

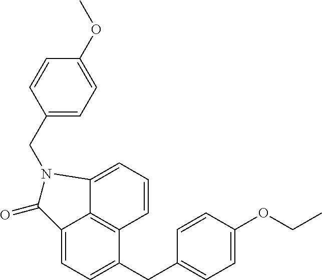 [SMILES: CCOc1ccc(Cc2ccc3c4c(cccc24)N(Cc2ccc(OC)cc2)C3=O)cc1]

Step 1: 5-(4-ethoxybenzyl)-1-(4-methoxybenzyl)benzo[cd]indol-2(1H)-one (170 mg, 54% yield) was obtained from general procedure B step 1. LC-MS (ES⁺): m/z 424.16 [M+H]⁺.

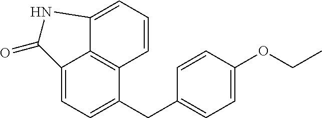 [SMILES: CCOc1ccc(Cc2ccc3c4c(cccc24)NC3=O)cc1]

Step 2: 5-(4-ethoxybenzyl)benzo[cd]indol-2(1H)-one (65 mg, 25% yield) was obtained from general procedure B step 2. LC-MS (ES⁺): m/z 304.13 [M+H]⁺.

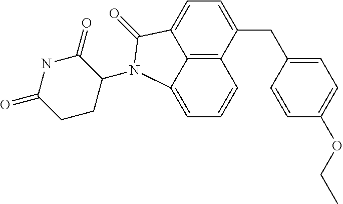 [SMILES: CCOc1ccc(Cc2ccc3c4c(cccc24)N(C2CCC(=O)NC2=O)C3=O)cc1]

Step 3: 3-(5-(4-ethoxybenzyl)-2-oxobenzo[cd]indol-1(2H)-yl)piperidine-2,6-dione Compound 74 (6 mg, 7% yield) was obtained from general procedure B step 3 as an off-white solid. LC-MS (ES⁻): m/z 413.40 [M−H].

### Example 60. Synthesis of 3-(5-(4-ethoxy-2-fluorobenzyl)-2-oxobenzo[cd]indol-1(2H)-yl)piperidine-2,6-dione (Compound 60)

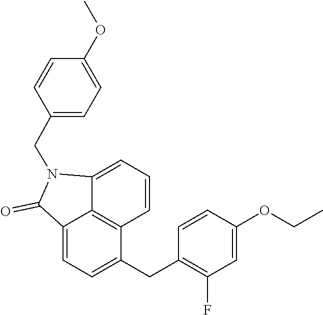 [SMILES: CCOc1ccc(Cc2ccc3c4c(cccc24)N(Cc2ccc(OC)cc2)C3=O)c(F)c1]

Step 1: 5-(4-ethoxy-2-fluorobenzyl)-1-(4-methoxybenzyl)benzo[cd]indol-2(1H)-one (260 mg, 75% yield) was obtained from general procedure B step 1. LC-MS (ES⁺): m/z 442.0 [M+H]⁺.

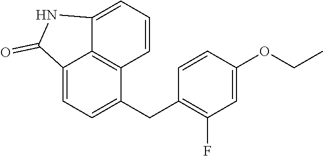 [SMILES: CCOc1ccc(Cc2ccc3c4c(cccc24)NC3=O)c(F)c1]

Step 2: 5-(4-ethoxy-2-fluorobenzyl)benzo[cd]indol-2(1H)-one (140 mg, 40% yield) was obtained from general procedure B step 2. LC-MS (ES⁺): m/z 322.0 [M+H]⁺.

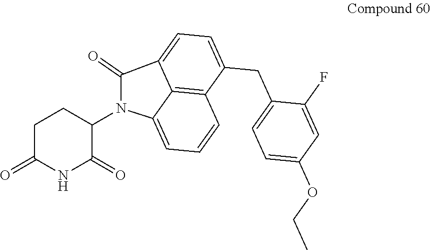 [SMILES: CCOc1ccc(Cc2ccc3c4c(cccc24)N(C2CCC(=O)NC2=O)C3=O)c(F)c1]

Step 3: 3-(5-(4-ethoxy-2-fluorobenzyl)-2-oxobenzo[cd]indol-1(2H)-yl)piperidine-2,6-dione Compound 75 (10 mg, 15% yield) was obtained from general procedure B step 3 as an off-white solid. LC-MS (ES⁺): m/z 433.38 [M+H]⁺.

### Example 61. Synthesis of ethyl 4-((1-(2,6-dioxopiperidin-3-yl)-2-oxo-1,2-dihydrobenzo[cd]indol-5-yl)methyl)benzoate (Compound 61)

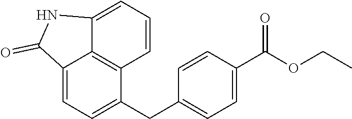 [SMILES: CCOC(=O)c1ccc(Cc2ccc3c4c(cccc24)NC3=O)cc1]

Step 1: ethyl 4-((2-oxo-1,2-dihydrobenzo[cd]indol-5-yl)methyl)benzoate (40 mg, 15% yield) was obtained from general procedure A step 1. LC-MS (ES⁺): m/z 332.03 [M+H]⁺.

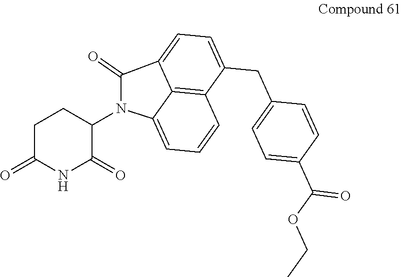 [SMILES: CCOC(=O)c1ccc(Cc2ccc3c4c(cccc24)N(C2CCC(=O)NC2=O)C3=O)cc1]

Step 2: ethyl 4-((1-(2,6-dioxopiperidin-3-yl)-2-oxo-1,2-dihydrobenzo[cd]indol-5-yl)methyl)benzoate Compound 61 (9 mg, 16% yield) was obtained from general procedure A step 2 as light yellow solid. LC-MS (ES⁻): m/z 441.35 [M−H]⁺.

### Example 62. Synthesis of 6-bromo-10,11-diazatricyclododeca-(4),1(6),2(5),3(10),7-pentaen-9-one

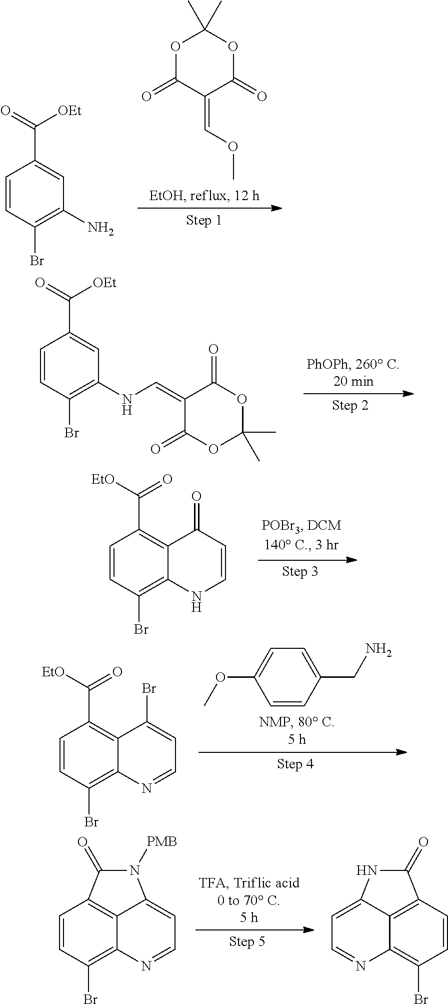

Step 1: To a well stirred solution of ethyl 3-amino-4-bromo-benzoate (20 g, 81.94 mmol) in ethanol (100 mL) was added 5-(methoxymethylene)-2,2-dimethyl-1,3-dioxane-4,6-dione (12.00 g, 64.46 mmol) and the reaction mixture was heated at 80° C. overnight. After completion, the solvent was removed under reduced pressure to obtain crude residue which was then washed with pentane followed by 50% Et₂O/Pentane to afford ethyl 4-bromo-3-[(2,2-dimethyl-4,6-dioxo-1,3-dioxan-5-ylidene)methylamino]benzoate (25 g, 50.23 mmol, 61% yield) as yellow solid. ¹H NMR (400 MHz, DMSO-d₆) δ 11.51 (d, J=13.8 Hz, 1H), 8.74 (t, J=7.2 Hz, 1H), 8.22 (d, J=1.16 Hz, 1H), 7.91 (d, J=8.32 Hz, 1H), 7.74-7.71 (m, 1H), 4.39-4.33 (q, 2H), 1.7 (s, 6H), 1.34 (t, J=7.08 Hz, 1H).

Step 2: Solution of ethyl 4-bromo-3-[(2,2-dimethyl-4,6-dioxo-1,3-dioxan-5-ylidene)methylamino]benzoate (20 g, 50.23 mmol) in Ph₂O (40 mL) was heated at 260° C. for 20 minutes. The reaction mass was cooled to room temperature and poured into hexane. The resulting semi solid was filtered and washed with hexane, followed by 50% pentane/Et₂O several time to afford ethyl 8-bromo-4-oxo-1H-quinoline-5-carboxylate (12 g, 31.61 mmol, 63% yield) which was used for the next step without further purification. LC-MS (ES⁺): m/z 296.24 [M+H]⁺.

Step 3: A solution of ethyl 8-bromo-4-oxo-1H-quinoline-5-carboxylate (12 g, 40.52 mmol) and phosphoryl bromide (69.71 g, 243.15 mmol, 24.72 mL) in HPLC grade DCM (25 mL) was heated at 140° C. for 3 hours. After completion, the reaction mix was diluted with DCM (200 mL) and washed with saturated NaHCO₃ solution followed by brine solution. The organic phase was separated, dried over anhydrous sodium sulfate, filtered, and evaporated under reduced pressure. The resulting crude mass was purified by column chromatography (hexane to 100% DCM as eluent) to give ethyl 4,8-dibromoquinoline-5-carboxylate (8.5 g, 23.68 mmol, 70% yield) as a colorless solid. LC-MS (ES⁺): m/z 360.15 [M+H]⁺.

Step 4: To a solution of ethyl 4,8-dibromoquinoline-5-carboxylate (5.5 g, 15.32 mmol) in HPLC grade NMP (30 mL) was added 4-methoxybenzylamine (4.20 g, 30.64 mmol, 4.00 mL) and the reaction mixture was heated at 80° C. for 5 h. After completion, the reaction was diluted with ethyl acetate (200 mL) and then washed with water and brine. The organic layer was separated, dried over anhydrous sodium sulfate, filtered and then concentrated under reduced pressure to yield a crude residue which was then purified by silica-gel column chromatography to afford 14-bromo-19-[(4-methoxyphenyl)methyl]-18,19-diazatricyclododeca-5(12),6(14),7(13),8(18),15-pentaen-17-one (4.5 g, 9.99 mmol, 65% yield) as white solid. LC-MS (ES⁺): m/z 371.1 [M+H]⁺.

Step 5: To the solid compound 14-bromo-19-[(4-methoxyphenyl)methyl]-18,19-diazatricyclododeca-5(12),6(14),7(13),8(18),15-pentaen-17-one (4 g, 10.83 mmol) was added TFA (10.0 mL) followed by trifluoromethanesulfonic acid (16.26 g, 108.34 mmol, 9.51 mL) at 0° C. and stirred for 30 minutes at the same temperature. The reaction mixture was further allowed to heat at 70° C. for 5 hours. After completion, the reaction was diluted with DCM (150 mL) and slowly poured into ice-cold water. The resulting solution was then neutralized with Na₂CO₃ solution. The organic phase was separated, dried over anhydrous sodium sulfate, filtered, and then concentrated under reduced pressure to give the crude residue which was then purified by silica gel column chromatography to get 6-bromo-10,11-diazatricyclododeca-(4),1(6),2(5),3(10),7-pentaen-9-one (2 g, 4.58 mmol, 42% yield) as white solid. LC-MS (ES⁺): m/z 248.8 [M+H]⁺.

### Example 63. Synthesis of 3-(8-(1-(3-(morpholinosulfonyl)benzyl)piperidin-4-yl)-5-oxopyrrolo[2,3,4-de]quinolin-4(5H)-yl)piperidine-2,6-dione (Compound 77)

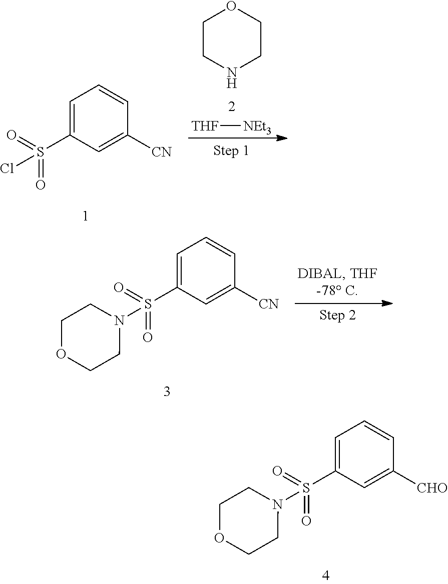

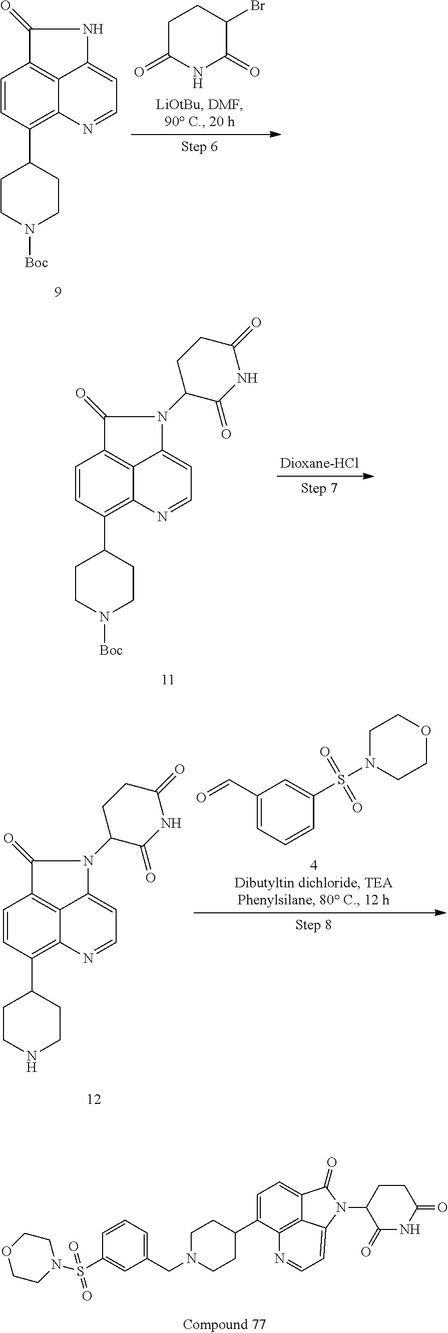

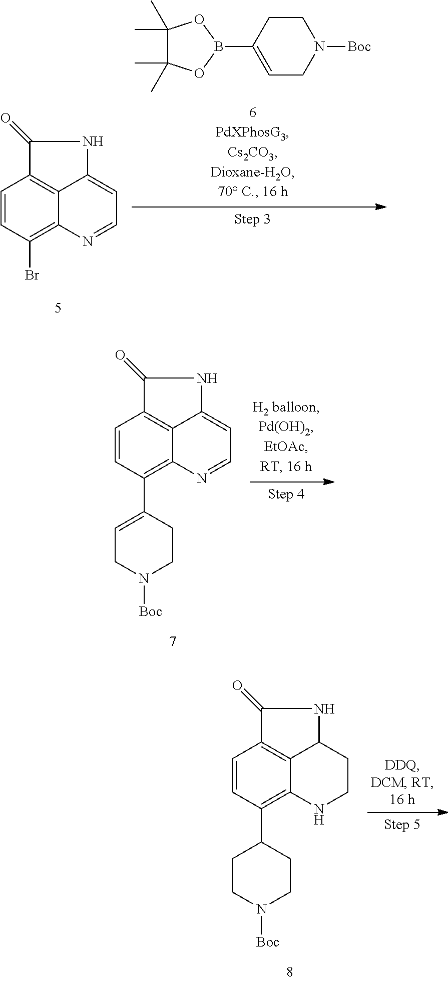 [SMILES: CC(C)(C)OC(=O)N1CC=C(B2OC(C)(C)C(C)(C)O2)CC1 ; O=C1Nc2ccnc3c(Br)ccc1c23 ; CC(C)(C)OC(=O)N1CC=C(c2ccc3c4c(ccnc24)NC3=O)CC1 ; CC(C)(C)OC(=O)N1CCC(c2ccc3c4c2NCCC4NC3=O)CC1]

Step 1: In a flame dried 100 mL round-bottom flask under nitrogen atmosphere, 3-cyanobenzenesulfonyl chloride 1 (1.8 g, 8.93 mmol) was dissolved in dry THE (20 mL) and cooled down to 0° C. To this solution, triethyl amine (1.81 g, 17.85 mmol, 2.49 mL) was added followed by the addition of morpholine 2 (933.29 mg, 10.71 mmol, 937.04 μL) under inert atmosphere. Resulting reaction mixture was warmed to room temperature and stirred for 12 hr. After completion of reaction as evidenced from TLC, volatiles were removed under vacuum and crude was directly subjected to flash chromatography to afford 3-morpholinosulfonylbenzonitrile 3 (1.85 g, 5.97 mmol, 67% yield). LC-MS (ES⁺): m/z 253.27 [M+H]⁺.

Step 2: To the stirred solution of 3-morpholinosulfonylbenzonitrile 3 (500 mg, 1.98 mmol) in dry THF (200 mL) was added DIBAL-H (2.03 g, 3.57 mmol, 2.89 mL) drop wise at 0° C. and stirred for another 16 hours at room temperature. After completion of the reaction (monitored by TLC), the reaction mixture was diluted with ethyl acetate (100 mL) and quenched with saturated solution of rochelle's salt. Resulting turbid solution was stirred for 2 hours until clear aqueous-organic layer separation was observed. The organic layer was separated, dried over anhydrous sodium sulphate and evaporated under reduced pressure to obtain the crude compound which was purified by flash chromatography using 0-10% ethyl acetate-DCM to afford 3-morpholinosulfonylbenzaldehyde 4 (200 mg, 783.42 μmol, 40% yield) as colorless gum. LC-MS (ES⁺): m/z 256.13 [M+H]⁺.

Step 3: To a well degassed solution of 6-bromo-10,11-diazatricyclododeca-,2(5),3(10),4(7),6(8)-pentaen-9-one 5 (500 mg, 2.01 mmol), tert-butyl 4-(4,4,5,5-tetramethyl-1,3,2-dioxaborolan-2-yl)-3,6-dihydro-2H-pyridine-1-carboxylate 6 (744.89 mg, 2.41 mmol) in dioxane (6 mL)-Water (1.5 mL), Cesium carbonate (1.64 g, 5.02 mmol) was added followed by XPhos Pd G3 (254.89 mg, 301.13 μmol). Resulting reaction mixture was heated at 90° C. for 16 h. After completion of reaction, reaction mixture was diluted with ethyl acetate (25 mL), filtered through a short pad of celite and washed with excess ethyl acetate. Combined organic part was washed with water, brine, dried over anhydrous sodium sulphate, filtered and concentrated under reduced pressure. Crude mass was purified by column chromatography to afford tert-butyl 4-(17-oxo-20,21-diazatricyclododeca-3,5(13),6(20),11(14),12(15)-pentaen-12-yl)-3,6-dihydro-2H-pyridine-1-carboxylate 7 (300 mg, 700.06 μmol, 35% yield). LC-MS (ES⁺): m/z 352 [M+H]⁺.

Step 4: To a degassed solution of tert-butyl 4-(17-oxo-20,21-diazatricyclododeca-3,5(13),6(20),11(14),12(15)-pentaen-12-yl)-3,6-dihydro-2H-pyridine-1-carboxylate 7 (0.3 g, 853.73 μmol) in Ethyl acetate (15 mL), dihydroxypalladium (0.27 g, 1.92 mmol) was added and resulting reaction mixture was hydrogenated with H2 balloon at room temperature for 16 h. After complete consumption of starting material as evidenced from LCMS, the reaction mixture was filtered through celite bed and washed with ethyl acetate (100 mL). The filtrate was collected and concentrated under reduced pressure. Crude reaction mass was purified by flash column chromatography using ethyl acetate-Hexanes (10-50%) as eluent to afford tert-butyl 4-(3-oxo-2,9-diazatricyclo[6.3.1.0^{4,12}]dodeca-4(12),5,7-trien-7-yl)piperidine-1-carboxylate 8 (220 mg, 615.48 μmol, 72% yield). LC-MS (ES⁺): m/z 358 [M+H]⁺.

Step 5: To a stirred solution of tert-butyl 4-(17-oxo-20,21-diazatricyclododeca-3,11(14),12-trien-11-yl)piperidine-1-carboxylate 8 (220 mg, 615.48 μmol) in HPLC grade DCM (12 mL), 2,3-Dichloro-5,6-dicyano-1,4-benzoquinone (153.68 mg, 677.01 μmol) was added drop wise at 0° C. After complete addition, reaction mixture was stirred at RT for 16 h. After completion of reaction (as monitored by TLC), the reaction mixture was diluted with DCM (30 mL) and washed with 1M NaOH solution followed by brine. Organic portion was separated, dried over sodium sulfate, concentrated under reduced pressure. Resulting crude reaction mass was purified by flash column chromatoraphy using 30% DCM-ethyl acetate mixture to have the desire compound tert-butyl 4-(17-oxo-20,21-diazatricyclododeca-3,5(13),6(20),11(15),12(14)-pentaen-11-yl)piperidine-1-carboxylate 9 (100 mg, 141.48 μmol, 23% yield). LC-MS (ES⁺): m/z 354 [M+H]⁺.

Step 6: To a cooled solution of tert-butyl 4-(17-oxo-20,21-diazatricyclododeca-3,5(13),6(20),11(15),12(14)-pentaen-11-yl)piperidine-1-carboxylate 9 (100 mg, 282.95 mol) in dry DMF (5 mL), Lithium tert-butoxide, 99.9% (metals basis) (90.61 mg, 1.13 mmol) was added under inert atmosphere, maintaining the temp <5° C. Once the addition is over, the resultant mixture was stirred for 15 minutes at room temperature. Then the reaction mixture was again cooled to 0° C. and 3-bromopiperidine-2,6-dione 10 (108.66 mg, 565.91 μmol) was added to it. After complete addition, resulting solution was heated at 90° C. for 16 hours. After formation of new spot (evidenced from TLC), the reaction mixture was cooled to 0° C. and quenched with the addition of saturated NH₄Cl solution. The aqueous layer was extracted with ethyl acetate (3×50 mL). Combined organics was separated, dried over anhydrous sodium sulfate, and concentrated under reduced pressure. Crude reaction mass was purified by flash column chromatography using DCM-Ethyl acetate (1:1, v/v) as eluent to afford tert-butyl 4-[28-(2,6-dioxo-3-piperidyl)-21-oxo-25,28-diazatricyclododeca-3,5(15),6(25),13(17),14(16)-pentaen-13-yl]piperidine-1-carboxylate 11 (25 mg, 38.75 μmol, 14% yield). LC-MS (ES⁺): m/z 465 [M+H]⁺.

Step 7: To a stirred solution of tert-butyl 4-[28-(2,6-dioxo-3-piperidyl)-21-oxo-25,28-diazatricyclododeca-3,5(15),6(25),13(17),14(16)-pentaen-13-yl]piperidine-1-carboxylate 11 (25 mg, 53.82 μmol) in HPLC grade dioxane (0.5 mL), dioxane-HCl (4 M, 30 μL) was added drop wise in ice-cold condition. After complete addition, resulting reaction mixture was stirred at room temperature for 3 hours. After complete consumption of starting material (as evidenced from LCMS), the volatiles were removed under reduced pressure to afford crude 3-[18-oxo-10-(4-piperidyl)-20,23-diazatricyclododeca-,2(12),3(20),10(14),11(13)-pentaen-23-yl]piperidine-2,6-dione hydrochloride 12 (15 mg, 37.42 μmol, 70% yield) which was used in next step without any purification. LC-MS (ES⁺): m/z 365 [M+H]⁺.

Step 8: To the stirred solution of 3-[18-oxo-10-(4-piperidyl)-20,23-diazatricyclododeca-,2(12),3(20),10(14),11(13)-pentaen-23-yl]piperidine-2,6-dione hydrochloride 12 (15 mg, 37.42 μmol) in dry THF(3 mL), triethylamine (7.57 mg, 74.84 μmol, 10.43 μL) was added (pH˜7) followed by 3-morpholinosulfonylbenzaldehyde 4 (9.55 mg, 37.42 mol) and dibutyl tin dichloride (13.64 mg, 44.90 μmol, 10.03 μL). Resulting reaction mixture was heated for 1 hour at 60° C. After that, the reaction mixture was cooled to room temperature and phenylsilane (6.07 mg, 56.13 μmol) was carefully added to it and again heated at 80° C. for 12 hours. After completion of the reaction as confirmed by LC MS, the reaction mixture was concentrated and crude material was purified by was purified by reverse-phase prep HPLC to afford 3-[21-[1-[(3-morpholinosulfonylphenyl)methyl]-4-piperidyl]-29-oxo-31,35-diazatricyclododeca-3(21),4(22),5(23),6(31),24-pentaen-35-yl]piperidine-2,6-dione Compound 77 (2.94 mg, 4.69 μmol, 13% yield); ¹H NMR (400 MHz, DMSO-d₆) δ 11.15 (s, 1H), 8.85 (d, J=4.72 Hz, 1H), 8.08 (d, J=7.28 Hz, 1H), 7.87 (d, J=7.36 Hz, 1H), 7.73 (br, 2H), 7.65-7.63 (br, 2H), 7.21 (d, J=4.76 Hz, 1H), 5.43 (dd, J1=12.92 Hz, J=5.32 Hz 1H), 3.82 (br m, 1H), 3.69 (s, 2H), 63.62 (t, J=4.24 Hz, 4H), 3.0 (m, 3H), 2.87 (t, J=4.32 Hz, 4H), 2.67-2.63 (m, 2H), 2.27-2.21 (m, 2H), 2.13-2.11 (m, 1H), 1.95-1.90 (m, 4H). LC-MS (ES⁺): m/z 604 [M+H]⁺.

### Example 64. Synthesis of (S)-4-(4-(4-((1-(2,6-dioxopiperidin-3-yl)-2-oxo-1,2-dihydrobenzo[cd]indol-6-yl)methyl)benzyl)piperazin-1-yl)-3-fluorobenzonitrile (Compound 78) and (R)-4-(4-(4-((1-(2,6-dioxopiperidin-3-yl)-2-oxo-1,2-dihydrobenzo[cd]indol-6-yl)methyl)benzyl)piperazin-1-yl)-3-fluorobenzonitrile (Compound 79)

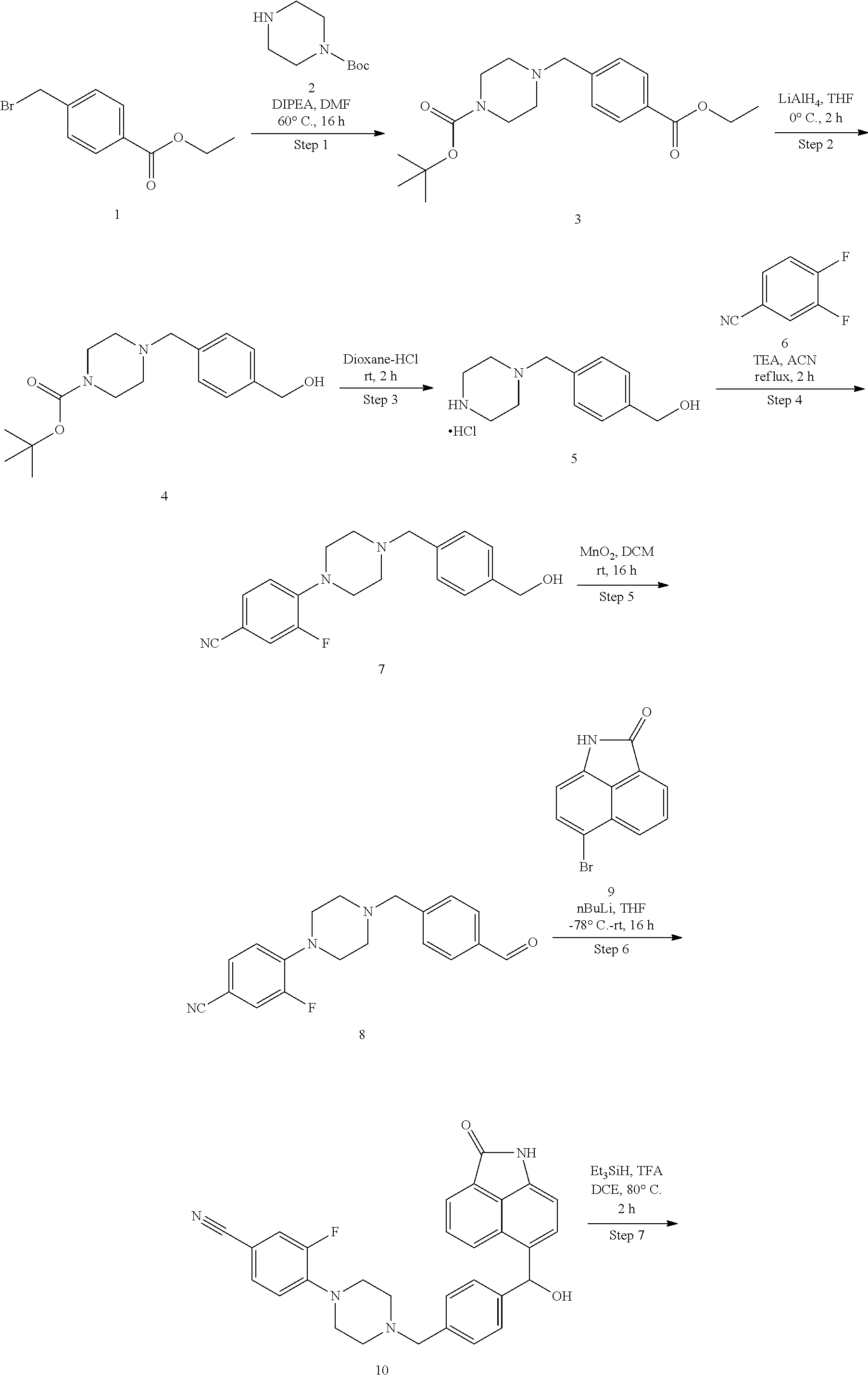

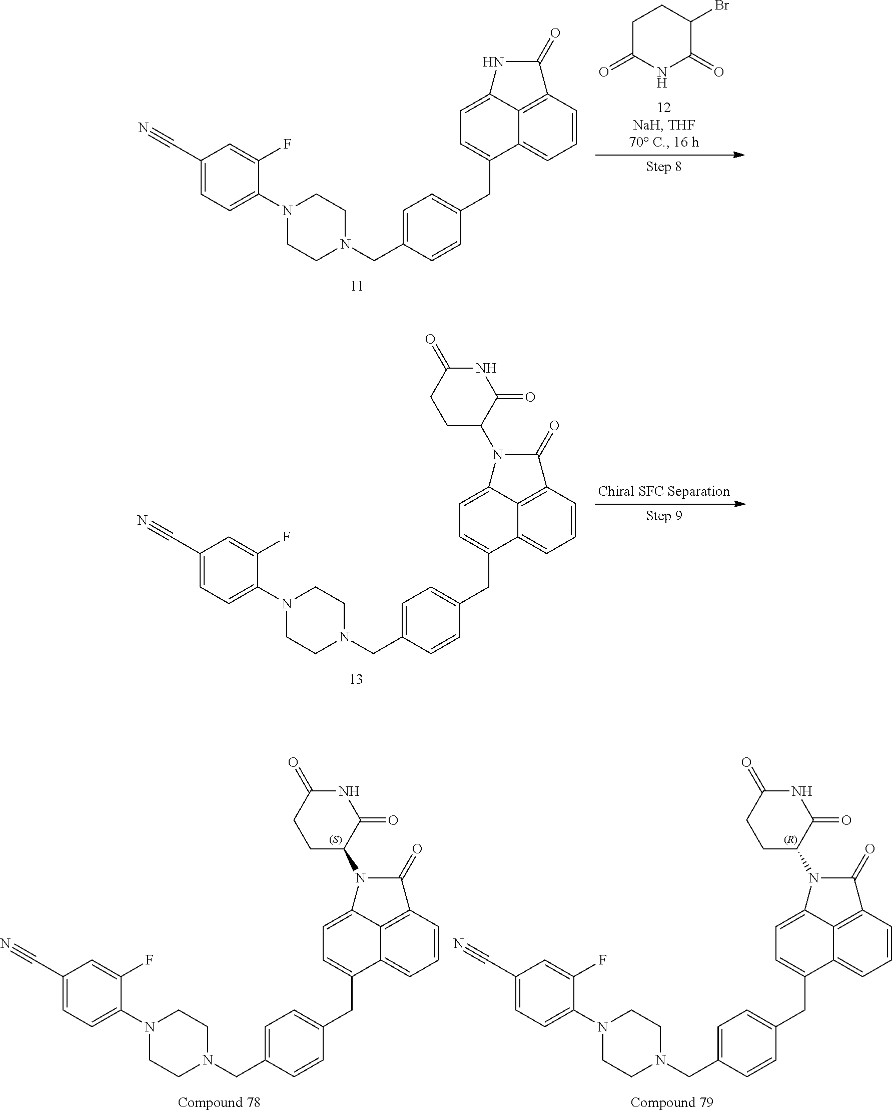

Step 1: To a stirred solution ethyl 4-(bromomethyl)benzoate 1 (5 g, 20.57 mmol) in DMF (50.0 mL) were added DIPEA (7.97 g, 61.70 mmol, 10.75 mL) and tert-butyl piperazine-1-carboxylate 2 (3.83 g, 20.57 mmol) under nitrogen atmosphere. The reaction mixture was refluxed at 60° C. for 16 hours. The reaction was quenched with water and extracted with ethyl acetate. Organic layer was separated, dried over anhydrous sodium sulphate and concentrated under reduced pressure to get crude compound. The crude product was purified by CombiFlash column using (0-15% EA/Hexanes) to get tert-butyl 4-(4-(ethoxycarbonyl)benzyl)piperazine-1-carboxylate 3 (5.6 g, 15.27 mmol, 74% yield) as a colorless gum. LC-MS (ES⁺): m/z 349.0 [M+H]⁺.

Step 2: To a stirred solution of tert-butyl 4-[(4 ethoxycarbonylphenyl)methyl]piperazine-1-carboxylate 3 (5 g, 14.35 mmol) in THE (60.0 mL) at 0° C. was added LiAlH4 (1.09 g, 28.70 mmol) slowly under nitrogen atmosphere and the reaction mixture was stirred for 2 hours in cold condition. Upon completion, the reaction mixture was quenched with (1.1 mL) water and (1.1 mL) 15% NaOH fallowed by (2.2 mL) water. It was then stirred for 30 minutes, filtered through celite bed and concentrated under reduced pressure to get tert-butyl 4-[[4-(hydroxymethyl)phenyl]methyl]piperazine-1-carboxylate 4 (4.35 g, 13.49 mmol, 94% yield). LC-MS (ES⁺): m/z 306.9 [M+H]⁺.

Step 3: To the stirred solution of tert-butyl 4-[[4-(hydroxymethyl)phenyl]methyl]piperazine-1-carboxylate 4 (4.35 g, 14.20 mmol) at cold condition was added 4M HCl in dioxane (16.00 g, 438.83 mmol, 20 mL) slowly under nitrogen atmosphere and the reaction mixture was stirred for 2 hours at room temperature. Upon completion, reaction was concentrated under reduced pressure, triturated with ether to get [4-(piperazin-1-ylmethyl)phenyl]methanol hydrochloride 5 (3.4 g, 10.96 mmol, 77% yield). LC-MS (ES⁺): m/z 207.3 [M+H]⁺.

Step 4: A stirred solution of [4-(piperazin-1-ylmethyl)phenyl]methanol hydrochloride 5 (2 g, 8.24 mmol) in acetonitrile (20.0 mL) were added TEA (2.87 mL, 20.60 mmol) and 3,4-difluorobenzonitrile 6 (1.15 g, 8.24 mmol) under nitrogen atmosphere. The reaction mixture was then refluxed for 2 hours. Upon completion, reaction mixture was diluted with water and extracted with ethyl acetate. Organic layer was separated, dried over anhydrous sodium sulphate, concentrated under reduced pressure to get crude compound. Crude thus obtained was purified by Combi-Flash chromatography using (0-60% EA/Hexanes) to get 3-fluoro-4-[4-[[4-(hydroxymethyl)phenyl]methyl]piperazin-1-yl]benzonitrile 7 (550 mg, 1.69 mmol, 20% yield) as off white solid. LC-MS (ES⁺): m/z 326.4 [M+H]⁺.

Step 5: To the stirred solution of 3-fluoro-4-[4-[[4-(hydroxymethyl)phenyl]methyl]piperazin-1-yl]benzonitrile 7 (600 mg, 1.84 mmol) in DCM (20.0 mL) was added MnO2 (1.60 g, 18.44 mmol) under nitrogen and stirred at room temperature for 16 hours. Upon completion, reaction mixture was filtered through celite bed. Filtrate was concentrated to afford 3-fluoro-4-(4-(4-formylbenzyl)piperazin-1-yl)benzonitrile 8 (473 mg, 78% yield). LC-MS (ES⁺): m/z 324.3 [M+H]⁺.

Step 6: To the stirred solution of 6-bromo-1H-benzo[cd]indol-2-one 9 (1.1 g, 4.43 mmol) in THE (10.0 mL) was added n-butyllithium (2.0 M, 4.88 mL) at −78° C. and after the addition was complete the temperature was allowed to increase to −40° C. The reaction mixture was stirred at the same temperature for 30 minutes followed by the addition of 3-fluoro-4-[4-[(4-formylphenyl)methyl]piperazin-1-yl]benzonitrile 8 (1.43 g, 4.43 mmol) in THE (10.0 mL) at −78° C. and then the reaction mixture was allowed to warm to room temperature. Reaction was then continued for 16 hours at room temperature. Upon completion, the reaction mixture was quenched with saturated aq. ammonium chloride solution, extracted with ethyl acetate. The organic layer was washed with water, dried over anhydrous sodium sulphate and evaporated under reduced pressure to obtain the crude compound which was purified by flash chromatography using (0-5% MeOH-DCM) to afford 3-fluoro-4-[4-[[4-[hydroxy-(2-oxo-1H-benzo[cd]indol-6-yl)methyl]phenyl]methyl]piperazin-1-yl]benzonitrile 10 (650.0 mg, 1.12 mmol, 25% yield) as brown solid. LC-MS (ES⁺): m/z 493.0 [M+H]⁺.

Step 7: To the stirred solution of 3-fluoro-4-[4-[[4-[hydroxy-(2-oxo-1H-benzo[cd]indol-6-yl)methyl]phenyl]methyl]piperazin-1-yl]benzonitrile 10 (650.0 mg, 1.32 mmol) in DCE (5.0 mL) were added triethylsilane (613.80 mg, 5.28 mmol, 843.13 μL) and trifluoroacetic acid (1.20 g, 10.56 mmol, 813.37 μL) and the reaction mixture was heated at 80° C. for 2 hours. Upon completion, reaction mixture was diluted with ethyl acetate and water and the organic fraction was separated. It was dried over anhydrous sodium sulphate and evaporated under reduced pressure to obtain the crude compound which was purified by flash chromatography using (0-5% MeOH-DCM) to afford 3-fluoro-4-[4-[[4-[(2-oxo-1H-benzo[cd]indol-6-yl)methyl]phenyl]methyl]piperazin-1-yl]benzonitrile 11 (400.0 mg, 686.61 μmol, 52% yield) as a brown solid. LC-MS (ES⁺): m/z 477.4 [M+H]⁺.

Step 8: To the stirred solution of 3-fluoro-4-[4-[[4-[(2-oxo-1H-benzo[cd]indol-6-yl)methyl]phenyl]methyl]piperazin-1-yl]benzonitrile 11 (400.0 mg, 839.38 μmol) in DMF (2.0 mL) was added sodium hydride (60% dispersion in mineral oil) (192.97 mg, 5.04 mmol) while maintaining at cold temperature and the reaction mixture was heated at 60° C. for 1 hour. Then to it was added 3-bromopiperidine-2,6-dione 12 (483.51 mg, 2.52 mmol) and the reaction was heated at 60° C. for 4 hours with further addition of 3-bromopiperidine-2,6-dione (483.51 mg, 2.52 mmol). The reaction was then continued for 16 hours at same temperature. The reaction mixture was diluted with ethyl acetate, it was added to citric acid solution (pH 5), washed with water and the organic fraction was separated. Organic part was dried over anhydrous sodium sulphate and evaporated under reduced pressure to obtain the crude which was purified by preparative TLC plate (eluting with 35% ethyl acetate-DCM) to afford 4-(4-(4-((1-(2,6-dioxopiperidin-3-yl)-2-oxo-1,2-dihydrobenzo[cd]indol-6-yl)methyl)benzyl)piperazin-1-yl)-3-fluorobenzonitrile 13 (70 mg) in the form of enantiomeric mixture.

¹H NMR (400 MHz, DMSO-d₆) δ 11.12 (s, 1H), 8.32 (d, J=8.28 Hz, 1H), 8.07 (d, J=6.92 Hz, 1H), 7.80 (t, J=7.66 Hz, 1H), 7.66 (d, J=12.4 Hz, 1H), 7.54 (d, J=8.36 Hz, 1H), 7.40 (d, J=7.28 Hz, 1H), 7.26-7.19 (m, 4H), 7.11-7.05 (m, 2H), 5.44 (dd, J=12.64, 4.84 Hz, 1H), 4.37 (s, 2H), 3.49 (s, 2H), 3.12 (br s, 4H), 2.98-2.90 (m, 1H), 2.79-2.73 (m, 1H), 2.70-2.62 (m, 1H), 2.45 (br s, 4H), 2.10-2.07 (m, 1H). LC-MS (ES⁺): m/z 588.5 [M+H]⁺.

Step 9: 70 mg of 4-[4-[[4-[[1-(2,6-dioxo-3-piperidyl)-2-oxo-benzo[cd]indol-6-yl]methyl]phenyl]methyl]piperazin-1-yl]-3-fluoro-benzonitrile 13 was separated into enantiomers by normal phase chiral HPLC to afford (S)-4-(4-(4-((1-(2,6-dioxopiperidin-3-yl)-2-oxo-1,2-dihydrobenzo[cd]indol-6-yl)methyl)benzyl)piperazin-1-yl)-3-fluorobenzonitrile Compound 78 (6.0 mg, 100% ee) and (R)-4-(4-(4-((1-(2,6-dioxopiperidin-3-yl)-2-oxo-1,2-dihydrobenzo[cd]indol-6-yl)methyl)benzyl)piperazin-1-yl)-3-fluorobenzonitrile Compound 79 (6.0 mg, 100% ee) as yellow solids.

### Example 65: 2-(4-chlorophenyl)-N-[[1-(2,6-dioxo-3-piperidyl)-2-oxo-benzo[cd]indol-5-yl]methyl]-2,2-difluoro-acetamide (Compound 80)

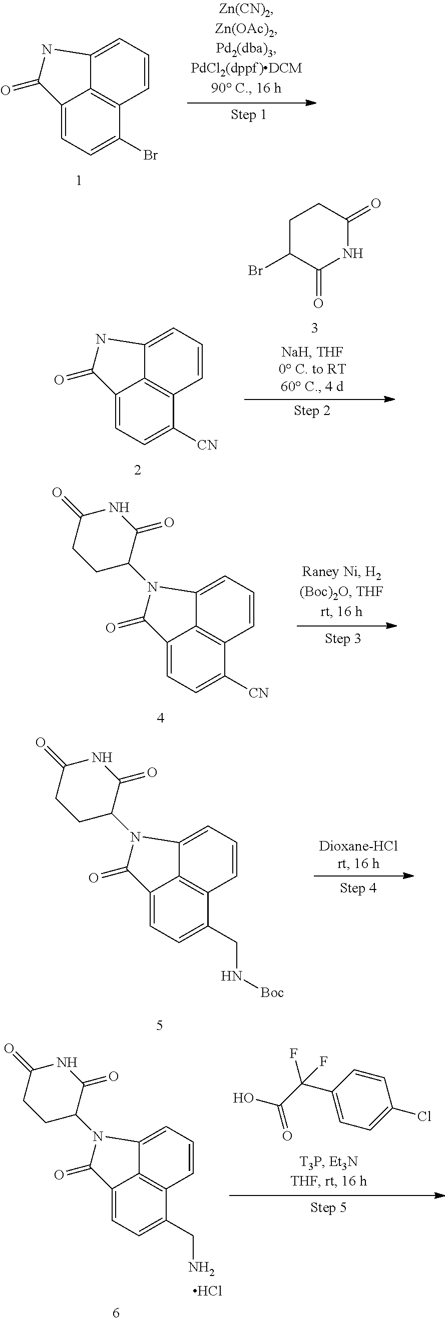

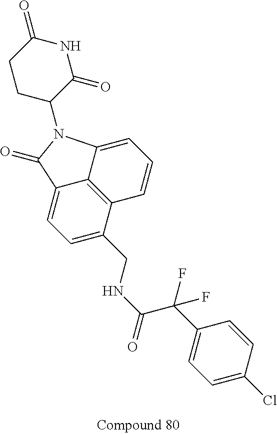 [SMILES: O=C1CCC(N2C(=O)c3ccc(CNC(=O)C(F)(F)c4ccc(Cl)cc4)c4cccc2c34)C(=O)N1]

Step 1: A stirred solution of 5-bromo-1H-benzo[cd]indol-2-one 1 (0.900 g, 3.63 mmol) in DMF (12.0 mL) in a sealed tube was degassed for 5 mins, later zinc cyanide (724.22 mg, 6.17 mmol, 391.47 μL) and zinc acetate (732.21 mg, 3.99 mmol) were added and again degassed for 5 mins, later tris(dibenzylideneacetone)dipalladium(0) (166.11 mg, 181.40 μmol) and [1,1′-Bis(diphenylphosphino)ferrocene]dichloropalladium(II), complex with dichloromethane (59.25 mg, 72.56 μmol) was added and again degassed for 5 mins, after degassing sealed tube was closed with teflon cap and stirred at 90° C. for 16 hours. The progress of the reaction was monitored by TLC, after reaction completion reaction mixture was diluted with ethyl acetate and water. Layers were separated, organic layer was washed with water, brine, dried over anhydrous sodium sulfate, filtered and concentrated under reduced pressure to get the crude compound and crude thus obtained was purified by column chromatography (eluted with 10 to 50% ethyl acetate in hexaness) to get 2-oxo-1H-benzo[cd]indole-5-carbonitrile 2 (0.390 g, 1.93 mmol, 53% yield) as yellow solid. LC-MS (ES⁺): m/z 193.0 [M+H]⁺.

Step 2: A stirred solution of 2-oxo-1H-benzo[cd]indole-5-carbonitrile 2 (0.370 g, 1.91 mmol) in DMF (5.0 mL) was cooled to 0° C. and stirred at 0° C. for 10 mins, later Sodium hydride (in oil dispersion) 60% dispersion in mineral oil (182.52 mg, 4.76 mmol) was added and stirred at 0° C. for 45 mins, after that 3-bromopiperidine-2,6-dione 3 (1.46 g, 7.62 mmol) was added by dissolved in DMF (5 mL) and stirred at room temperature for 30 mins. Reaction mixture was then stirred at 60° C. for 4 days. The progress of the reaction was monitored by TLC, and then reaction mixture was quenched with chilled water and extracted with ethyl acetate. Organic layer was washed with water, brine, dried over anhydrous sodium sulfate, filtered and concentrated under reduced pressure to get the crude compound and crude thus obtained was purified by column chromatography (eluted with 10 to 60% ethyl acetate in hexanes) to get 1-(2,6-dioxo-3-piperidyl)-2-oxo-benzo[cd]indole-5-carbonitrile 4 (100 mg, 308.82 μmol, 16% yield) as yellow solid. LC-MS (ES⁻): m/z 303.8 [M−H]⁺.

Step 3: To a stirred solution of 1-(2,6-dioxo-3-piperidyl)-2-oxo-benzo[cd]indole-5-carbonitrile 4 (0.085 g, 278.43 μmol) in THE (5.0 mL) was added di-tert-butyl dicarbonate (151.91 mg, 696.07 μmol, 159.74 μL) and followed by addition of Raney Nickel (0.120 g, 1.40 mmol) at room temperature. Reaction mixture was stirred at room temperature under hydrogen atmosphere for 16 hours. The progress of the reaction was monitored by TLC, after reaction completion reaction mixture was filtered through celite bed and bed was washed twice with ethyl acetate. Combined filtrate was concentrated under reduced pressure to get the crude compound and it was purified by column chromatography (eluted with 10 to 60% ethyl acetate in Hexanes) to get tert-butyl N-[[1-(2,6-dioxo-3-piperidyl)-2-oxo-benzo[cd]indol-5-yl]methyl]carbamate 5 (70 mg, 160.78 μmol, 58% yield) as light yellow solid. LC-MS (ES⁺): m/z 410.2 [M+H]⁺.

Step 4: To a stirred solution of tert-butyl N-[[1-(2,6-dioxo-3-piperidyl)-2-oxo-benzo[cd]indol-5-yl]methyl]carbamate 5 (0.060 g, 146.54 μmol) in 1,4-dioxane (3.0 mL) was added 4.0M hydrogen chloride solution in dioxane (133.58 mg, 3.66 mmol, 166.97 μL). The reaction mixture was stirred at room temperature for 16 hours. The progress of the reaction was monitored by TLC, after reaction completion reaction mixture was concentrated under reduced pressure to get the crude compound and it was triturated with ether and pentane to get 3-[5-(aminomethyl)-2-oxo-benzo[cd]indol-1-yl]piperidine-2,6-dione hydrochloride 6 (0.045 g, 123.63 μmol, 84% yield) as yellow solid. LC-MS (ES⁺): m/z 310.1 [M+H]⁺.

Step 5: To a stirred solution of 3-[5-(aminomethyl)-2-oxo-benzo[cd]indol-1-yl]piperidine-2,6-dione hydrochloride 6 (0.040 g, 115.68 μmol) in THE (5.0 mL) was added 2-(4-chlorophenyl)-2,2-difluoro-acetic acid 7 (23.90 mg, 115.68 μmol) under argon atmosphere then reaction mixture cooled to 0° C. then triethylamine (58.53 mg, 578.40 μmol, 80.62 μL) was added and followed by addition of propylphosphonic anhydride solution (184.04 mg, 289.20 μmol, 172.00 μL, 50% purity). Reaction mixture was then stirred at room temperature for 16 hours. The progress of the reaction was monitored by TLC, after reaction completion reaction mixture was diluted with ethyl acetate then gave sodium bicarbonate wash and followed by brine wash. Organic part was separated, dried over anhydrous sodium sulfate, filtered and concentrated under reduced pressure to get the crude compound and crude thus obtained was purified by column chromatography (eluted with 1 to 5% MeOH in DCM) to get 2-(4-chlorophenyl)-N-[[1-(2,6-dioxo-3-piperidyl)-2-oxo-benzo[cd]indol-5-yl]methyl]-2,2-difluoro-acetamide Compound 80 (20 mg, 39.21 μmol, 34% yield) as a yellow solid. ¹HNMR (400 MHz, DMSO-d₆): δ 11.12 (s, 1H), 9.79-9.78 (m, 1H), 8.05 (d, J=7.2 Hz, 1H), 7.75 (d, J=8.64 Hz, 1H), 7.64 (d, J=7.28 Hz, 1H), 7.61 (s, 4H), 7.51 (t, J=7.9 Hz, 1H), 7.16 (d, J=7.16 Hz, 1H), 5.44 (dd, J=12.68, 5.0 Hz, 1H), 4.89 (d, J=5.76 Hz, 2H), 2.95-2.93 (m, 1H), 2.76-2.73 (m, 1H), 2.66-2.63 (m, 1H), 2.10-2.09 (m, 1H). LC-MS (ES⁺): m/z 498.2 [M+H]⁺.

### Example 66: 3-[18-(1-benzyl-4-fluoro-4-piperidyl)-24-oxo-27,30-diazatricyclododeca-5(17),6(18),7(19),8(27),20-pentaen-30-yl]piperidine-2,6-dione (Compound 81)

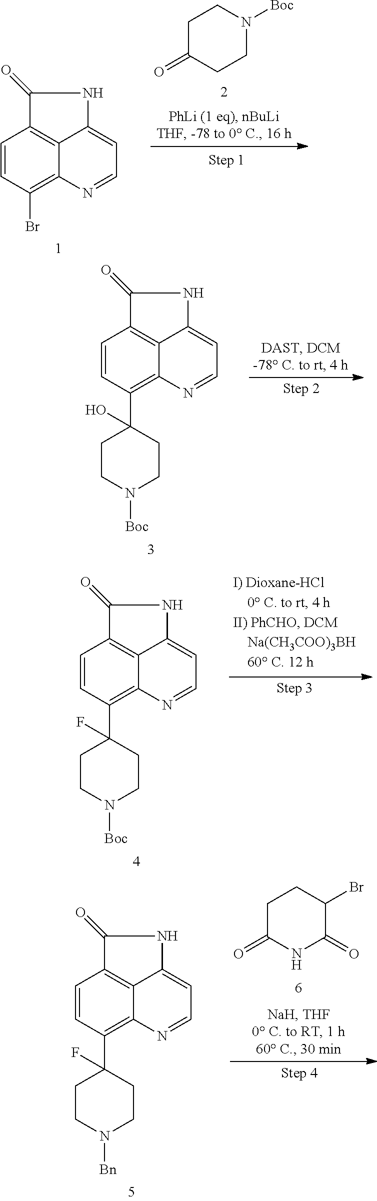 [SMILES: O=C1Nc2ccnc3c(Br)ccc1c23 ; CC(C)(C)OC(=O)N1CCC(=O)CC1 ; CC(C)(C)OC(=O)N1CCC(O)(c2ccc3c4c(ccnc24)NC3=O)CC1 ; CC(C)(C)OC(=O)N1CCC(F)(c2ccc3c4c(ccnc24)NC3=O)CC1 ; O=C1Nc2ccnc3c(C4(F)CCN(Cc5ccccc5)CC4)ccc1c23 ; O=C1CCC(Br)C(=O)N1]

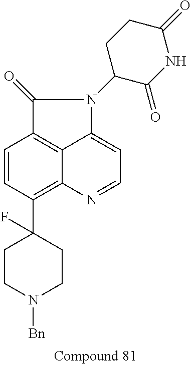 [SMILES: O=C1CCC(N2C(=O)c3ccc(C4(F)CCN(Cc5ccccc5)CC4)c4nccc2c34)C(=O)N1]

Step 1: To a flame-dried round-bottom flask with a magnetic stir under N2, 6-bromo-10,11-diazatricyclododeca-,2(5),3(10),4(7),6(8)-pentaen-9-one 1 (400 mg, 1.61 mmol) was dissolved in dry THF (10.0 mL) and the flask was cooled to −78° C. To this solution phenyllithium, 1.8 M in di-n-butyl ether (683.64 mg, 8.13 mmol, 844.00 μL) was added drop wise and resulting reaction mixture was stirred at same temperature for 30 minutes followed by the addition of butyllithium typically 2 M in Hexane (1.34 M, 882.00 μL) at −78° C. After complete addition, the temperature was allowed to increase to −40° C. and the reaction mixture was stirred at the same temperature for additional 30 minutes. A solution of tert-butyl 4-oxopiperidine-1-carboxylate 2 (319.99 mg, 1.61 mmol) in dry THF (10.0 mL) was added at −78° C. and then the reaction mixture was allowed to warm up to room temperature and stirred for 16 hours at the same temperature. After completion of reaction, the mixture was quenched with ammonium chloride solution and diluted with ethyl acetate (100 mL). The organic layer was washed with water/brine and separated, dried over anhydrous sodium sulphate and evaporated under reduced pressure to obtain the crude compound which was purified by flash chromatography using 0-5% MeOH-DCM to afford tert-butyl 4-hydroxy-4-(16-oxo-20,21-diazatricyclododeca-3,5(13),6(20),11(14),12(15)-pentaen-12-yl)piperidine-1-carboxylate 3 (500 mg, 947.45 μmol, 59% yield) as brown solid. LC-MS (ES⁺): m/z 370.4 [M+H]⁺.

Step 2: To a well stirred solution of tert-butyl 4-hydroxy-4-(16-oxo-20,21-diazatricyclododeca-3(11),4(12),5(13),6(20),14-pentaen-12-yl)piperidine-1-carboxylate 3 (300 g, 812.10 mmol) in anhydrous DCM (15.0 mL) was added N-ethyl-N-(trifluoro-$1{4}-sulfanyl)ethanamine (261.80 g, 1.62 mol, 214.59 mL) drop wise at −78° C. After complete addition, the reaction mixture was allowed to warm up to room temperature and was stirred for another 4 hours. After formation of new spot (as evidenced from TLC), the reaction mixture was poured slowly into ice-cold aqueous NaHCO₃ (sat.). The aqueous layer was extracted with DCM (3×20 mL). The organic layer was separated, dried over anhydrous sodium sulfate, concentrated, and dried in vacuum to afford crude tert-butyl 4-fluoro-4-(16-oxo-20,21-diazatricyclododeca-3(11),4(12),5(13),6(20),14-pentaen-12-yl)piperidine-1-carboxylate 4 (200 mg, 301.56 μmol, 4% yield) which was used in the next step without purification. LC-MS (ES⁺): m/z 372.4 [M+H]⁺.

Step 3: To the stirred solution of -butyl 4-fluoro-4-(16-oxo-20,21-diazatricyclododeca-3(11),4(12),5(13),6(20),14-pentaen-12-yl)piperidine-1-carboxylate 4 (200 mg, 301.56 μmol) in Dioxane (4 mL), 4 M Dioxane-HCl was added (9.04 mmol, 2.0 mL) at 0° C. and the reaction mass was stirred at RT for 4 h. After completion of reaction (as evidenced from LC-MS), volatiles were removed under reduced pressure and crude mass was washed with pentane/diethyl ether and dried well to afford 9-(4-fluoro-4-piperidyl)-15,17-diazatricyclododeca-(8),1(9),2(10),3(15),11-pentaen-13-one (109 mg, 401.79 μmol) which was redissolved in dry DCM (5.0 mL) and neutralized with triethylamine (pH˜7). To this solution benzaldehyde (85.28 mg, 803.57 μmol, and 82.00 μL) was added followed by acetic acid (48.25 mg, 803.57 μmol, and 45.96 μL) and stirred at 60° C. for 2 hr. After 2 hours, reaction mixture was cooled to room temperature and sodium; triacetoxyboranuide (425.77 mg, 2.01 mmol) was added to it and stirring was continued for another 12 hr. After completion of reaction (as evidenced from crude LC MS), volatiles were removed under vacuum and the resulting mixture was extracted with ethyl acetate (40 mL). Organic phase was washed with water/brine and separated, dried over sodium sulfate and concentrated under reduced pressure to afford the crude material which was subjected to flash chromatography using (30-40% EtOAc/DCM as eluent) to afford 16-(1-benzyl-4-fluoro-4-piperidyl)-22,23-diazatricyclododeca-5(15),6(16),7(17),8(22),18-pentaen-20-one 5 (90 mg, 209.18 μmol, 52% yield) as brownish gummy. LC-MS (ES⁺): m/z 362.2 [M+H]⁺.

Step 4: To a chilled solution of 16-(1-benzyl-4-fluoro-4-piperidyl)-22,23-diazatricyclododeca-5(15),6(16),7(17),8(22),18-pentaen-20-one 5 (57.76 mg, 159.82 μmol) in dry THE (5 mL) was added sodium hydride (in oil dispersion) 60% dispersion in mineral oil (153.09 mg, 4.00 mmol) portion wise, maintaining the temp <5° C. Once the addition is over, the resultant mixture was stirred for 15 minutes at room temperature. Then the reaction mixture was again cooled to 0° C. and 3-bromopiperidine-2,6-dione 6 (368.24 mg, 1.92 mmol) was added to it portion wise. After complete addition, resulting solution was heated at 70° C. 1 hr. After consumption of the starting material, the reaction mixture was cooled to 0° C. and quenched with ice-cold water (5 mL). Aqueous part was extracted with ethyl acetate (3×50 mL). Combined organics was separated, dried over anhydrous sodium sulfate and concentrated under reduced pressure. Crude was purified by PREP-TLC to afford 3-[18-(1-benzyl-4-fluoro-4-piperidyl)-24-oxo-27,30-diazatricyclododeca-5(17),6(18),7(19),8(27),20-pentaen-30-yl]piperidine-2,6-dione Compound 81 (26.3 mg, 55.66 μmol, 35% yield). ¹H NMR (400 MHz, DMSO-d₆) δ 11.17 (s, 1H), 8.92 (d, J=4.84 Hz, 1H), 8.15 (d, J=7.36 Hz, 1H), 8.05 (d, J=7.4 Hz, 1H), 7.38-7.34 (m, 4H), 7.29-7.24 (m, 2H), 5.44 (dd, J1=11.28, J2=3.32 Hz, 1H), 3.59 (s, 2H), 3.28-3.19 (m, 2H), 2.85-2.64 (m, 5H), 2.49-41 (m, 2H), 2.07 (m, 1H), 1.88-1.82 (m, 2H). LC-MS (ES⁺): m/z 473.3 [M+H]⁺.

### Example 67: 2-(2,6-dioxo-3-piperidyl)-2,7-diazatricyclo[6.3.1.04, 12]dodeca-1(11),4,8(12),9-tetraene-3,6-dione (Compound 82)

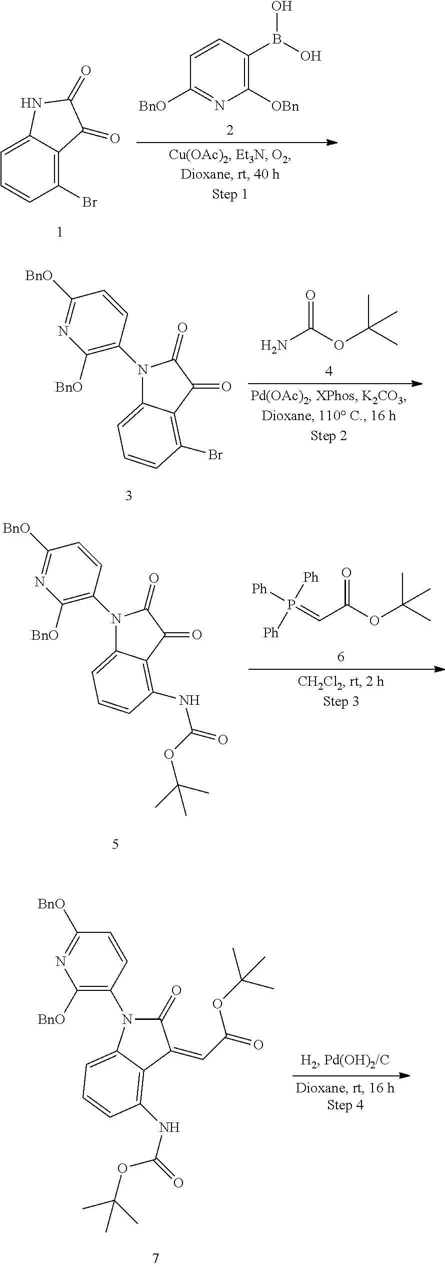

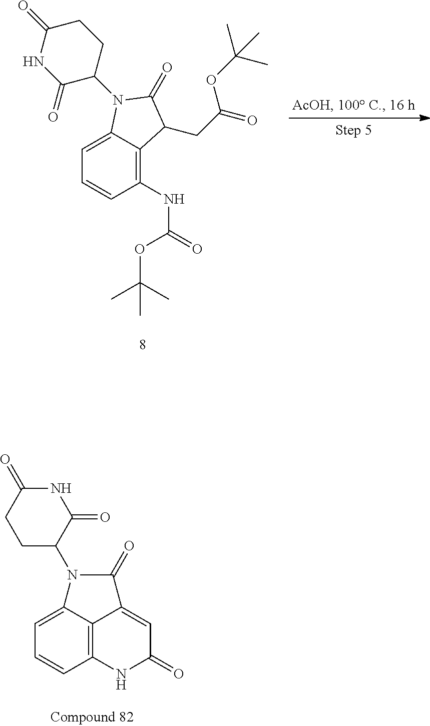 [SMILES: CC(C)(C)OC(=O)CC1C(=O)N(C2CCC(=O)NC2=O)c2cccc(NC(=O)OC(C)(C)C)c21 ; O=C1CCC(N2C(=O)c3cc(=O)[nH]c4cccc2c34)C(=O)N1]

Step 1: To a solution of 4-bromoindoline-2,3-dione 1 (700 mg, 3.10 mmol) in 1,4-dioxane (40 mL), were added (2,6-dibenzyloxy-3-pyridyl)boronic acid 2 (2.08 g, 6.19 mmol), copper(II) acetate (1.13 g, 6.19 mmol) and triethylamine (940.15 mg, 9.29 mmol, 1.29 mL). The resulting mixture was stirred at room temperature under oxygen atmosphere for 40 h. The reaction mixture was diluted with ethyl acetate (100 mL), washed with water (20 mL) and the organic phase was separated. The organic phase was washed with brine solution (20 mL) and dried over anhydrous sodium sulfate. The solution was filtered and concentrated under reduced pressure to give the crude product. The crude product was purified by flash chromatography (silica gel, 230-400 mesh) eluted with 8-15% ethyl acetate in petroleum ether to give 4-bromo-1-(2,6-dibenzyloxy-3-pyridyl)indoline-2,3-dione 3 (620 mg, 1.12 mmol, 36% yield) as a red solid. LC-MS (ES⁺): m/z 515.0 [M+H]⁺.

Step 2: To a solution of 4-bromo-1-(2,6-dibenzyloxy-3-pyridyl)indoline-2,3-dione 3 (620 mg, 1.12 mmol) and tert-butyl carbamate 4 (525.91 mg, 4.49 mmol) in 1,4-dioxane (12 mL), was added potassium carbonate (310.22 mg, 2.24 mmol). The contents were degassed under nitrogen for 5 min. To this mixture, were added 2-dicyclohexylphosphino-2′,4′,6′-triisopropylbiphenyl (107.01 mg, 224.47 μmol) and palladium(II) acetate (50.39 mg, 224.47 μmol). The contents were heated at 110° C. for 16 h. The reaction mixture was filtered through a pad of celite and washed with ethyl acetate (50 mL). The filtrate was concentrated under reduced pressure to give the crude product. The crude product was purified by flash chromatography (silica gel, 230-400 mesh) to give tert-butyl N-[1-(2,6-dibenzyloxy-3-pyridyl)-2,3-dioxo-indolin-4-yl]carbamate 5 (600 mg, 1.06 mmol, 95% yield) as a red colored solid. LC-MS (ES⁺): m/z 496.2 [M−Isobutene+H]⁺.

Step 3: To a solution of tert-butyl N-[1-(2,6-dibenzyloxy-3-pyridyl)-2,3-dioxo-indolin-4-yl]carbamate 5 (210 mg, 372.08 μmol) in dichloromethane (3 mL), was added (tert-butoxycarbonylmethylene)triphenylphosphorane 6 (140.06 mg, 372.08 μmol) in dichloromethane (2 mL) drop-wise. The resulting mixture was stirred at room temperature for 2 h. The reaction mixture was concentrated under reduced pressure to give the crude product. The crude product was purified by flash chromatography (silica gel, 230-400 mesh) eluted with 5-8% ethyl acetate in petroleum ether to give tert-butyl 2-[4-(tert-butoxycarbonylamino)-1-(2,6-dibenzyloxy-3-pyridyl)-2-oxo-indolin-3-ylidene]acetate 7 (170 mg, 236.32 μmol, 64% yield) as a red colored solid. LC-MS (ES⁺): m/z 650.3 [M+H]⁺.

Step 4: To a solution of tert-butyl (2Z)-2-[4-(tert-butoxycarbonylamino)-1-(2,6-dibenzyloxy-3-pyridyl)-2-oxo-indolin-3-ylidene]acetate 7 (170 mg, 236.32 μmol) in 1,4-dioxane (4 mL), was added palladium hydroxide on carbon (5%, 85 mg). The contents were stirred at room temperature under hydrogen atmosphere for 16 h. The UPLC analysis of the crude mixture showed the formation of tert-butyl 2-[4-(tert-butoxycarbonylamino)-1-(2,6-dioxo-3H-pyridin-3-yl)-2-oxo-indolin-3-yl]acetate. The reaction mixture was filtered through a pad of celite and washed with ethyl acetate (25 mL). The filtrate was concentrated under reduced pressure to give the residue which was dissolved in 1,4-dioxane (4 mL), was added palladium hydroxide on carbon (5%, 85 mg). The contents were stirred at room temperature for 16 h under hydrogen atmosphere. The reaction mixture was filtered through a pad of celite and washed with ethyl acetate (25 mL). The filtrate was concentrated under reduced pressure to give the crude product. The crude product was purified by flash chromatography (silica gel, 230-400 mesh) eluted with 50-60% ethyl acetate in petroleum ether to give tert-butyl 2-[4-(tert-butoxycarbonylamino)-1-(2,6-dioxo-3-piperidyl)-2-oxo-indolin-3-yl]acetate 8 (70 mg, 142.64 μmol, 60% yield) as an off-white solid. LC-MS (ES⁺): m/z 362.4 [M−(2×Isoprene)+H]⁺.

Step 5: tert-Butyl 2-[4-(tert-butoxycarbonylamino)-1-(2,6-dioxo-3-piperidyl)-2-oxo-indolin-3-yl]acetate 8 (70 mg, 142.64 μmol) was taken in acetic acid (2 mL). The resulting mixture was heated at 100° C. for 16 h. The reaction mixture was concentrated under reduced pressure to give the crude product. The crude product was purified by preparative HPLC [Column: X select C18 (150×10) mm, 5 microns; Mobile phase: A: 0.1% Formic acid in water, B: Acetonitrile] and the fractions containing the compound was lyophilized to give 17-(2,6-dioxo-3-piperidyl)-15,17-diazatricyclododeca-,2(7),3(8),6(10)-tetraene-9,13-dione Compound 82 (7.4 mg, 24.39 μmol, 17% yield) as a pale yellow solid. LC-MS (ES⁺): m/z 298.0 [M+H]⁺. ¹H NMR (400 MHz, DMSO-d₆): δ 12.11 (s, 1H), 11.16 (s, 1H), 7.49-7.45 (m, 1H), 6.99 (s, 1H), 6.91 (d, J=8.4 Hz, 1H), 6.82 (d, J=7.6 Hz, 1H), 5.39-5.34 (m, 1H), 2.97-2.88 (m, 1H), 2.72-2.61 (m, 2H), 2.11-2.06 (m, 1H) ppm.

### Example 68. 3-(7-methoxy-3-oxo-2,6-diazatricyclo[6.3.1.04, 12]dodeca-1(12),4,6,8,10-pentaen-2-yl)piperidine-2,6-dione (Compound 83)

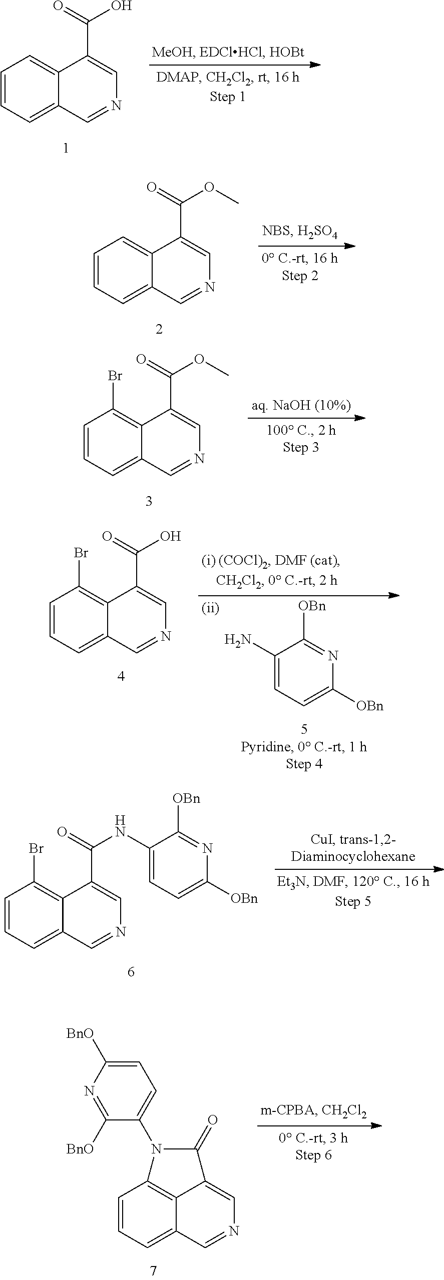

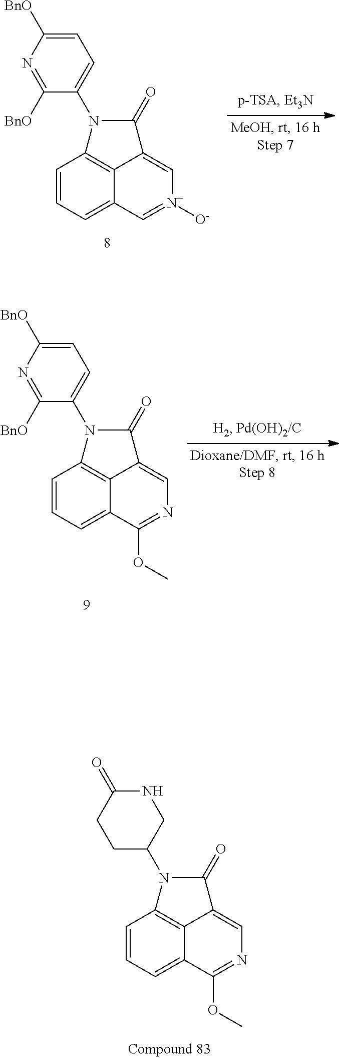 [SMILES: O=C1c2c[n+]([O-])cc3cccc(c23)N1c1ccc(OCc2ccccc2)nc1OCc1ccccc1 ; COc1ncc2c3c(cccc13)N(c1ccc(OCc3ccccc3)nc1OCc1ccccc1)C2=O ; COc1ncc2c3c(cccc13)N(C1CCC(=O)NC1)C2=O]

Step 1: To a solution of isoquinoline-4-carboxylic acid 1 (6.4 g, 36.96 mmol) in dichloromethane (75 mL), were added EDCl.HCl (8.50 g, 44.35 mmol), HOBt (5.99 g, 44.35 mmol) and DMAP (451.52 mg, 3.70 mmol). The resulting mixture was stirred at room temperature for 20 min. To this mixture, was added methanol (1.78 g, 55.44 mmol, 2.25 mL) and the resulting reaction mixture was stirred at room temperature for 16 h. The reaction mixture was treated with dichloromethane (120 mL) and water (15 mL). The organic phase was separated and dried over anhydrous sodium sulfate. The solution was filtered and concentrated under reduced pressure to give the crude product. The crude product was purified by flash chromatography (silica gel, 230-400 mesh) eluted with 30-35% ethyl acetate in petroleum ether to give methyl isoquinoline-4-carboxylate 2 (6 g, 32.05 mmol, 87% yield) as a pale-yellow solid. LC-MS (ES⁺): m/z 188.2 [M+H]⁺.

Step 2: Methyl isoquinoline-4-carboxylate 2 (6 g, 30.91 mmol) was taken in sulfuric acid (35 mL), cooled to 0° C., was added N-bromosuccinimide (7.15 g, 40.19 mmol, 3.41 mL). The resulting mixture was stirred at room temperature for 16 h. The reaction mixture was treated with ice and slowly added sodium bicarbonate portion-wise. After neutralization, the reaction mixture was diluted with ethyl acetate (150 mL) and the organic phase was separated. The organic phase was washed with brine solution (40 mL) and dried over anhydrous sodium sulfate. The solution was filtered and concentrated under reduced pressure to give the crude product. The crude product was purified by flash chromatography (silica gel, 230-400 mesh) eluted with 20-25% ethyl acetate in petroleum ether to give methyl 5-bromoisoquinoline-4-carboxylate 3 (4.7 g, 15.53 mmol, 50% yield) as a yellow liquid. LC-MS (ES⁺): m/z 267.8 [M+H]⁺.

Step 3: To a solution of methyl 5-bromoisoquinoline-4-carboxylate 3 (4.5 g, 14.87 mmol) in methanol (23 mL), was added 10% aqueous NaOH solution (594.95 mg, 14.87 mmol, 67.5 mL). The resulting mixture was heated at 100° C. for 2 h. The reaction mixture was concentrated under reduced pressure to give the residue which was neutralized using potassium bisulfate solution at 0° C. and extracted using 10% methanol in dichloromethane (3×70 mL). The combined organics were washed with brine solution (40 mL) and dried over anhydrous sodium sulfate. The solution was filtered and concentrated under reduced pressure to give 5-bromoisoquinoline-4-carboxylic acid 4 (2.5 g, 9.28 mmol, 62% yield) as a yellow solid. LC-MS (ES⁺): m/z 250.0 [M−H]⁺.

Step 4: To a solution of 5-bromoisoquinoline-4-carboxylic acid 4 (2 g, 7.42 mmol) in dichloromethane (40 mL), cooled to 0° C., were added oxalyl chloride (1.22 g, 9.65 mmol, 841.71 L) and N,N-dimethylformamide (54.25 mg, 742.19 μmol, 57.46 μL). The resulting mixture was stirred at room temperature for 2 h. The reaction mixture was concentrated under nitrogen atmosphere at reduced pressure to give crude 5-bromoisoquinoline-4-carbonyl chloride as a yellow solid. The crude acid chloride was taken in dichloromethane (40 mL), cooled to 0° C., was added 2,6-dibenzyloxypyridin-3-amine 5 (5.68 g, 18.55 mmol) in pyridine (30 mL) drop-wise. The resulting mixture was stirred at room temperature for 1 h. The reaction mixture was concentrated under reduced pressure and treated with water (10 mL) and extracted with ethyl acetate (3×40 mL). The combined organics were washed with brine solution (40 mL) and dried over anhydrous sodium sulfate. The solution was filtered and concentrated under reduced pressure to give the crude product. The crude product was purified by flash chromatography (silica gel, 230-400 mesh) eluted with 40-50% ethyl acetate in petroleum ether to give 5-bromo-N-(2,6-dibenzyloxy-3-pyridyl)isoquinoline-4-carboxamide 6 (2.8 g, 4.95 mmol, 67% yield) as a brown solid. LC-MS (ES⁺): m/z 540.1 [M+H]⁺.

Step 5: To a solution of 5-bromo-N-(2,6-dibenzyloxy-3-pyridyl)isoquinoline-4-carboxamide 6 (2 g, 3.53 mmol) in N,N-dimethylformamide (50 mL), were added trans-1,2-diaminocyclohexane (403.51 mg, 3.53 mmol) and triethylamine (1.07 g, 10.60 mmol, 1.48 mL). The contents were purged with nitrogen for 5 min, was added copper(I) iodide (672.98 mg, 3.53 mmol). The contents were heated at 120° C. for 16 h. The reaction mixture was concentrated under reduced pressure and treated with water (10 mL) and extracted with ethyl acetate (3×40 mL). The combined organics were washed with brine solution (30 mL) and dried over anhydrous sodium sulfate. The solution was filtered and concentrated under reduced pressure to give the crude product. The crude product was purified by flash chromatography (silica gel, 230-400 mesh) eluted with 40-50% ethyl acetate in petroleum ether to give 1-(2,6-bis(benzyloxy)pyridin-3-yl)pyrrolo[4,3,2-de]isoquinolin-2(1H)-one 7 (1.3 g, 2.67 mmol, 76% yield) as a brown solid. LC-MS (ES⁺): m/z 460.1 [M+H]⁺.

Step 6: To a solution of 1-(2,6-bis(benzyloxy)pyridin-3-yl)pyrrolo[4,3,2-de]isoquinolin-2(1H)-one 7 (320 mg, 591.68 μmol) in dichloromethane (8 mL), cooled to 0° C., was added 70% 3-chloroperoxybenzoic acid (291.73 mg, 1.18 mmol). The resulting mixture was stirred at room temperature for 3 h. The reaction mixture was treated with ice water (1 mL) and extracted with dichloromethane (3×15 mL). The combined organics were washed with 10% aqueous sodium bicarbonate solution (10 mL) and dried over anhydrous sodium sulfate. The solution was filtered and concentrated under reduced pressure to give the crude product. The crude product was purified by flash chromatography (silica gel, 230-400 mesh) eluted with 10-12% methanol in dichloromethane to give 2-(2,6-dibenzyloxy-3-pyridyl)-6-oxido-2-aza-6-azoniatricyclo[6.3.1.04, 12]dodeca-1(12),4,6,8,10-pentaen-3-one 8 (120 mg, 186.15 μmol, 31% yield) as a brown solid. LC-MS (ES⁺): m/z 476.2 [M+H]⁺.

Step 7: To a solution of 2-(2,6-dibenzyloxy-3-pyridyl)-6-oxido-2-aza-6-azoniatricyclo[6.3.1.04, 12]dodeca-1(12),4,6,8,10-pentaen-3-one 8 (120 mg, 186.15 μmol) in methanol (2 mL), cooled to 0° C., were added p-toluenesulfonyl chloride (46.14 mg, 241.99 μmol) and triethylamine (37.67 mg, 372.29 μmol, 51.89 μL). The resulting mixture was stirred at room temperature for 16 h. The reaction mixture was concentrated under reduced pressure, treated with 10% aqueous sodium bicarbonate solution (2 mL) and extracted with dichloromethane (3×10 mL). The combined organics were washed with brine solution (5 mL) and dried over anhydrous sodium sulfate. The solution was filtered and concentrated under reduced pressure to give the crude product. The crude product was purified by flash chromatography (silica gel, 230-400 mesh) eluted with 20-25% ethyl acetate in petroleum ether to give 2-(2,6-dibenzyloxy-3-pyridyl)-7-methoxy-2,6-diazatricyclo[6.3.1.04, 12]dodeca-1(12),4,6,8,10-pentaen-3-one 9 (40 mg, 49.68 μmol, 27% yield) as a brown solid. LC-MS (ES⁺): m/z 490.0 [M+H]⁺.

Step 8: To a solution of 2-(2,6-dibenzyloxy-3-pyridyl)-7-methoxy-2,6-diazatricyclo[6.3.1.04, 12]dodeca-1(12),4,6,8,10-pentaen-3-one 9 (40 mg, 49.68 μmol) in 1,4-dioxane (2 mL) and N,N-dimethylformamide (2 mL), was added palladium hydroxide (20% on carbon, 6.98 mg). The contents were stirred at room temperature under hydrogen atmosphere for 16 h. The reaction mixture was filtered through a pad of celite and washed with mixture of 1,4-dioxane and N,N-dimethylformamide. The filtrate was concentrated under reduced pressure to give the crude product. The crude product was purified by preparative HPLC [Column: X Bridge C₈ (150×19 mm), 5 micron; Mobile phase: A: 0.1% HCOOH in water, B: Acetonitrile] and the fractions containing the compound was lyophilized to give 3-(7-methoxy-3-oxo-2,6-diazatricyclo[6.3.1.04, 12]dodeca-1(12),4,6,8,10-pentaen-2-yl)piperidine-2,6-dione Compound 83 (7.5 mg, 23.24 μmol, 47% yield) as an off-white solid. LC-MS (ES⁺): m/z 312.2 [M+H]⁺. ¹H NMR (400 MHz, DMSO-d₆): δ 11.15 (s, 1H), 8.74 (s, 1H), 7.69 (d, J=8.4 Hz, 1H), 7.60 (t, J=8.4 Hz, 1H), 7.34 (d, J=7.2 Hz, 1H), 5.48-5.44 (m, 1H), 4.20 (s, 3H), 2.97-2.91 (m, 1H), 2.77-2.73 (m, 1H), 2.64 (d, J=1.6 Hz, 1H), 2.12-2.08 (m, 1H) ppm.

### Example 69. 2-(2,6-dioxo-3-piperidyl)-2,6-diazatricyclo[6.3.1.04, 12]dodeca-1(12),4,8,10-tetraene-3,7-dione (Compound 84)

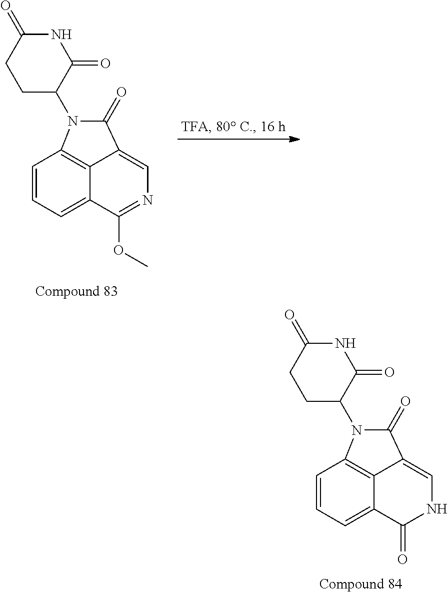 [SMILES: COc1ncc2c3c(cccc13)N(C1CCC(=O)NC1=O)C2=O ; O=C1CCC(N2C(=O)c3c[nH]c(=O)c4cccc2c34)C(=O)N1]

3-(7-Methoxy-3-oxo-2,6-diazatricyclo[6.3.1.04, 12]dodeca-1(12),4,6,8,10-pentaen-2-yl)piperidine-2,6-dione Compound 83 (7 mg, 21.69 μmol) was taken in trifluoroacetic acid (0.7 mL). The resulting mixture was heated at 80° C. for 16 h. The reaction mixture was concentrated under reduced pressure and co-distilled with methyl tert-butyl ether (1 mL) to give the crude product. The crude product was purified by reverse phase C18 column [Redisep 15.5 g C18 column, Mobile phase: A: 0.1% HCOOH in water, B: Acetonitrile] and the fractions containing the product was lyophilized to give 2-(2,6-dioxo-3-piperidyl)-2,6-diazatricyclo[6.3.1.04, 12]dodeca-1(12),4,8,10-tetraene-3,7-dione Compound 84 (3.1 mg, 10.33 mol, 48% yield) as an off-white solid. LC-MS (ES⁻): m/z 296.2 [M−H]⁺. ¹H NMR (400 MHz, DMSO-d₆): δ 12.07 (d, J=4.8 Hz, 1H), 11.12 (s, 1H), 8.22 (d, J=5.6 Hz, 1H), 7.59 (d, J=8.0 Hz, 1H), 7.45 (t, J=8.0 Hz, 1H), 7.27 (d, J=7.6 Hz, 1H), 5.40 (s, 1H), 2.93 (m, 1H), 2.72 (s, 1H), 2.68-2.66 (m, 1H), 2.01 (m, 1H) ppm.

### Example 70. 3-(9-methoxy-3-oxo-2,10-diazatricyclo[6.3.1.04, 12]dodeca-1(11),4,6,8(12),9-pentaen-2-yl)piperidine-2,6-dione (Compound 85)

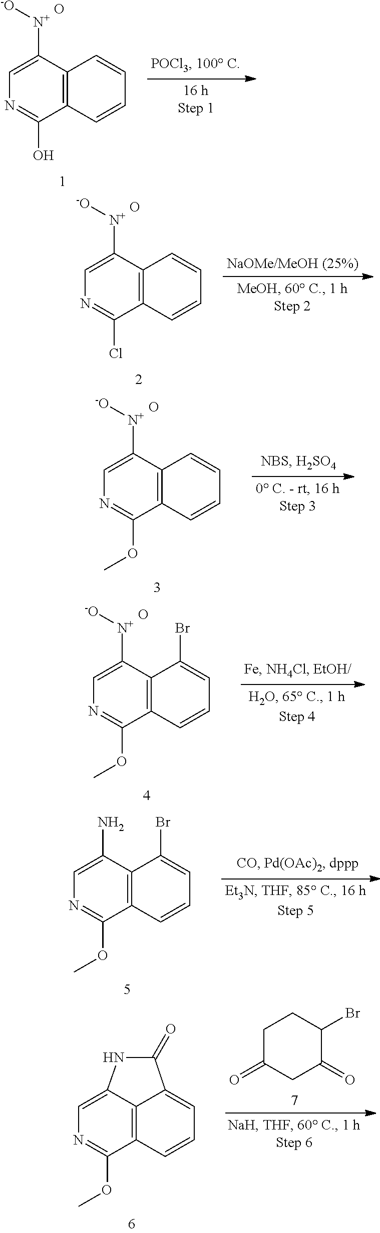

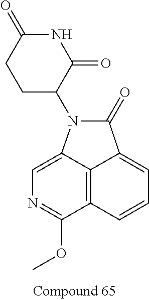 [SMILES: COc1ncc2c3c(cccc13)C(=O)N2C1CCC(=O)NC1=O]

Step 1: 4-Nitroisoquinolin-1-ol 1 (1 g, 5.26 mmol) was taken in phosphoryl chloride (10 g, 65.22 mmol) and the resulting mixture was heated to 100° C. for 16 h. The reaction mixture was concentrated under reduced pressure and the crude residue was treated with ice cold water. The precipitated solid was filtered, washed with water and dried under vacuum to give 1-chloro-4-nitro-isoquinoline 2 (1.05 g, 3.76 mmol, 72% yield) as an off-white solid. LC-MS (ES⁺): m/z 209.0 [M+H]⁺.

Step 2: To a solution of 1-chloro-4-nitro-isoquinoline 2 (1 g, 4.79 mmol) in methanol (13.66 mL), was added sodium methoxide, 25% in methanol (1.55 g, 28.76 mmol, 1.60 mL). The reaction mixture was stirred at 60° C. for 1 h. The reaction mixture was warmed to room temperature, treated with water (30 mL) and extracted with ethyl acetate (3×70 mL). The combined organics were washed with brine solution (50 mL) and dried anhydrous sodium sulfate. The solution was filtered and concentrated under reduced pressure to give 1-methoxy-4-nitro-isoquinoline 3 (950 mg, 4.19 mmol, 87.37% yield) as a pale-yellow solid. LC-MS (ES⁺): m/z 205.2 [M+H]⁺.

Step 3: 1-Methoxy-4-nitro-isoquinoline 3 (800 mg, 3.92 mmol) was taken in sulfuric acid (10 mL), cooled to 0° C., was added N-bromosuccinimide (906.54 mg, 5.09 mmol, 432.10 μL). The resulting mixture was stirred at room temperature for 16 h. The reaction mixture was treated with ice at 0° C. and slowly added sodium bicarbonate portion-wise. After neutralization, the reaction mixture was extracted with ethyl acetate (3×150 mL), The combined organics were washed with brine solution (50 mL) and dried over anhydrous sodium sulfate. The solution was filtered and concentrated under reduced pressure to give the crude product. The crude product was purified by flash chromatography using (silica gel, 230-400 mesh) eluted with 5-10% ethyl acetate in petroleum ether to give 5-bromo-1-methoxy-4-nitro-isoquinoline (550 mg, 1.77 mmol, 45% yield) as an off-white solid. LCMS (ES⁺): m/z 283.0 [M+H]⁺, RT=1.06 min and 8-bromo-1-methoxy-4-nitro-isoquinoline 4 (100 mg, 250.81 μmol, 6% yield) as an off-white solid. LC-MS (ES⁺): m/z 283.0 [M+H]⁺.

Step 4: To a solution of 5-bromo-1-methoxy-4-nitro-isoquinoline 4 (550 mg, 1.94 mmol) in ethanol (10 mL) and water (10 mL), were added iron powder (542.56 mg, 9.71 mmol) and ammonium chloride (519.64 mg, 9.71 mmol, 339.63 μL). The contents were heated at 65° C. for 1 h. The reaction mixture was filtered through a pad of celite, washed with ethyl acetate (80 mL) and water (30 mL), then separated the organic layer was dried over anhydrous sodium sulfate, filtered and concentrated under reduced pressure to get 5-bromo-1-methoxy-isoquinolin-4-amine 5 (460 mg, 1.64 mmol, 84% yield) as a yellow solid. LC-MS (ES⁺): m/z 253.0 [M+H]⁺.

Step 5: To a solution of 5-bromo-1-methoxy-isoquinolin-4-amine 5 (440 mg, 1.74 mmol) in THE (20 mL) were added palladium(II) acetate (195.15 mg, 869.24 μmol), 1,3-bis(diphenylphosphino)propane (215.11 mg, 521.54 μmol) and triethylamine (527.75 mg, 5.22 mmol, 726.93 μL). The contents were heated at 85° C. in an atmosphere of carbon monoxide (5.5 kg/cm²) for 16 hours. The reaction mixture was filtered through a pad of celite, and the filtrate was concentrated under reduced pressure to give the crude product. The crude product was purified by flash chromatography (silica gel, 230-400 mesh) eluted with 0-100% ethyl acetate in petroleum ether while the desired product eluting at 40% ethyl acetate in petroleum ether to give 9-methoxy-2,10-diazatricyclo[6.3.1.04, 12]dodeca-1(11),4,6,8(12),9-pentaen-3-one 6 (130 mg, 614.50 μmol, 35% yield) as an yellow solid. LC-MS (ES⁺): m/z 201.0 [M+H]⁺.

Step 6: To a solution of 9-methoxy-2,10-diazatricyclo[6.3.1.04, 12]dodeca-1(11),4,6,8(12),9-pentaen-3-one 6 (130 mg, 649.37 μmol) in THE (16 mL), cooled to 0° C., was added sodium hydride (60% dispersion in mineral oil, 89.57 mg, 3.90 mmol). The contents were stirred at 0° C. for 1 h. The mixture was cooled to 0° C., was added 3-bromopiperidine-2,6-dione 7 (374.06 mg, 1.95 mmol) in THF (6 mL) drop-wise. The contents were heated at 60° C. for 3 h. The reaction mixture was treated with cold water and aqueous ammonium chloride (20 mL) and extracted with ethyl acetate (3×50 mL). The combined organics were washed with brine solution (40 mL) and dried over anhydrous sodium sulfate. The solution was filtered and concentrated under reduced pressure to give the crude product. The crude product was purified by flash chromatography (silica gel, 230-400 mesh) eluted with 0-100% ethyl acetate in petroleum ether while the desired compound eluting at 60-70% ethyl acetate in petroleum ether to give 3-(9-methoxy-3-oxo-2,10-diazatricyclo[6.3.1.04, 12]dodeca-1(11),4,6,8(12),9-pentaen-2-yl)piperidine-2,6-dione Compound 85 (63 mg, 193.28 μmol, 30% yield) as a yellow solid. LC-MS (ES⁺): m/z 312.0 [M+H]⁺. ¹H NMR (400 MHz, DMSO-d₆): δ 11.14 (s, 1H), 8.35 (s, 1H), 8.33 (d, J=1.6 Hz, 1H), 7.94 (t, J=7.2 Hz, 1H), 7.76 (s, 1H), 5.49-5.44 (m, 1H), 4.06 (s, 3H), 3.00-2.92 (m, 1H), 2.80-2.76 (m, 1H), 2.68-2.63 (m, 1H), 2.14-2.10 (m, 1H) ppm.

### Example 71. 2-(2,6-dioxo-3-piperidyl)-2,10-diazatricyclo[6.3.1.04, 12]dodeca-1(11),4,6,8(12)-tetraene-3,9-dione (Compound 86)

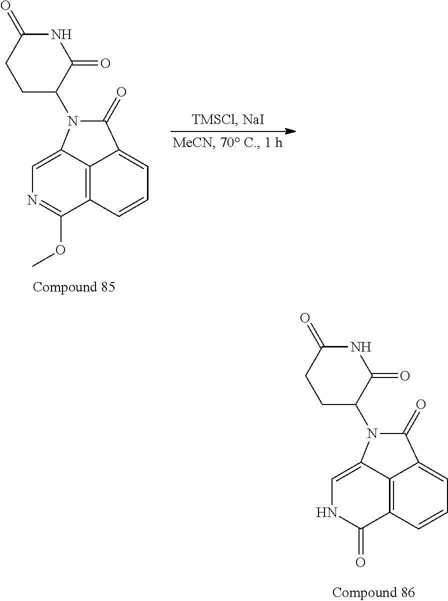 [SMILES: COc1ncc2c3c(cccc13)C(=O)N2C1CCC(=O)NC1=O ; O=C1CCC(N2C(=O)c3cccc4c(=O)[nH]cc2c34)C(=O)N1]

To a solution of 3-(9-methoxy-3-oxo-2,10-diazatricyclo[6.3.1.04, 12]dodeca-1(11),4,6,8(12),9-pentaen-2-yl)piperidine-2,6-dione Compound 85 (45 mg, 144.56 μmol) in acetonitrile (58.46 mL), were added sodium iodide (43.34 mg, 289.12 μmol, 11.82 μL) and chlorotri(methyl)silane (31.41 mg, 289.12 μmol). The resulting solution was heated at 70° C. for 1 hour. The reaction mixture was concentrated under reduced pressure to give the residue which was treated with 5% aqueous sodium thiosulfate (10 mL). The precipitated solid was filtered, washed with water and dried under vacuum to give 2-(2,6-dioxo-3-piperidyl)-2,10-diazatricyclo[6.3.1.04, 12]dodeca-1(11),4,6,8(12)-tetraene-3,9-dione Compound 86 (23 mg, 75.55 μmol, 52% yield) as a yellow solid. LC-MS (ES⁻): m/z 296.0 [M−H]⁺. ¹HNMR (400 MHz, DMSO-d₆): δ 11.21 (d, J=5.6 Hz, 1H), 11.08 (s, 1H), 8.28 (dd, J=7.8, 0.8 Hz, 1H), 8.23 (d, J=6.8 Hz, 1H), 7.81 (t, J=7.6 Hz, 1H), 7.20 (d, J=6.0 Hz, 1H), 5.39-5.34 (m, 1H), 2.93-2.85 (m, 1H), 2.77-2.70 (m, 1H), 2.68-2.59 (m, 1H), 2.07-2.02 (m, 1H) ppm.

### Example 72. 3-[5-(4,6-dimethylpyrimidin-2-yl)-2-oxo-benzo[cd]indol-1-yl]piperidine-2,6-dione (Compound 87)

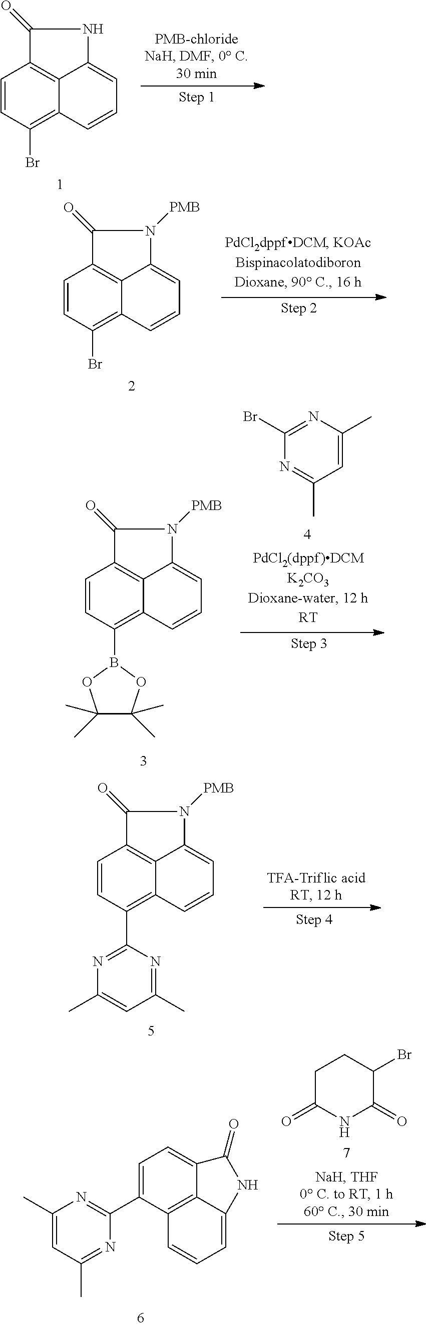

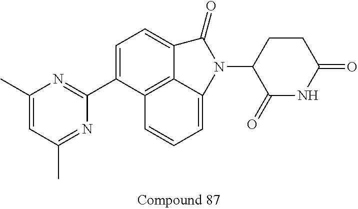 [SMILES: Cc1cc(C)nc(-c2ccc3c4c(cccc24)N(C2CCC(=O)NC2=O)C3=O)n1]

Step 1: To a stirred solution of 5-bromo-1H-benzo[cd]indol-2-one 1 (6.0 g, 24.19 mmol) in dry DMF (10.0), sodium hydride (60% dispersion in mineral oil) (1.39 g, 36.28 mmol) was added at 0° C. The reaction mixture was stirred for 30 minutes at the same temperature under inert atmosphere. 1-(chloromethyl)-4-methoxy-benzene (4.55 g, 29.02 mmol, and 3.79 mL) was then added to the reaction mixture and stirred for another 30 min at room temperature. After complete consumption of the starting material (monitored by TLC), ethyl acetate (100 mL) was added to the reaction mixture. The organic layer was washed with cold water (3×30 mL) followed by brine solution to remove DMF. The organic layer was separated, dried over sodium sulfate, filtered and concentrated under reduced pressure. The crude product was purified by combi-flash to afford 5-bromo-1-[(4-methoxyphenyl)methyl]benzo[cd]indol-2-one 2 (6.0 g, 15.81 mmol, 65% yield) as yellowish solid. LC-MS (ES⁺): m/z 370.2 [M+H]⁺.

Step 2: In a oven dried sealed vial under nitrogen atmosphere, 5-bromo-1-[(4-methoxyphenyl)methyl]benzo[cd]indol-2-one 2 (3.0 g, 8.15 mmol) was dissolved in 1,4 dioxane (60 mL) and bis(pinacolato) diboron (3.10 g, 12.22 mmol) followed by well dried potassium acetate (2.40 g, 24.44 mmol, 1.53 mL) were added to it. The resultant reaction mass was degassed well with argon for 15 minutes. Cyclopentyl (diphenyl)phosphane dichloromethane dichloropalladium iron (665.34 mg, 814.72 μmol) was added to the reaction mixture and heated at 100° C. for 16 hours. After completion of the reaction (monitored by TLC), the reaction mixture was cooled to RT, filtered through a pad of celite, washed with excess Ethyl acetate. The combined filtrate was washed with cold water (2×40 mL), dried over sodium sulfate and concentrated under reduced pressure. The crude residue was purified by flash chromatography to afford 1-[(4-methoxyphenyl)methyl]-5-(4,4,5,5-tetramethyl-1,3,2-dioxaborolan-2-yl)benzo[cd]indol-2-one 3 (2.9 g, 6.98 mmol, 86% yield) as a yellowish solid. LC-MS (ES⁺): m/z 416.4 [M+H]⁺.

Step 3: A mixture of 1-[(4-methoxyphenyl)methyl]-5-(4,4,5,5-tetramethyl-1,3,2-dioxaborolan-2-yl)benzo[cd]indol-2-one 3 (200 mg, 481.59 μmol), 2-bromo-4,6-dimethyl-pyrimidine 4 (75.06 mg, 401.33 μmol) and Potassium carbonate, anhydrous, 99% (166.40 mg, 1.20 mmol, 72.66 μL) were suspended in a mixture of Dioxan (4 mL)−Water (1 mL). Resulting reaction mixture was degassed with argon for 10 minutes, followed by the addition of Pd(dppf)C₁₂.DCM (32.77 mg, 40.13 μmol) and stirred at RT for 12 h. After completion of the reaction (as monitored by LCMS), the reaction mass was filtered through filter cartridge and filtrate was evaporated to dryness. Resulting crude reaction mass was diluted with EtOAc (50 mL) and washed with water/brine. Organic phase was separated, dried over anhydrous sodium sulfate, filtered and concentrated under reduced pressure to get crude material 5-(4,6-dimethylpyrimidin-2-yl)-1-[(4-methoxyphenyl)methyl]benzo[cd]indol-2-one 5 (110 mg, 250.35 μmol, 62% yield) which was used for the next step reaction without further purification. LC-MS (ES⁺): m/z 396.4 [M+H]⁺.

Step 4: To the stirred solution of 5-(4,6-dimethylpyrimidin-2-yl)-1-[(4-methoxyphenyl)methyl]benzo[cd]indol-2-one 5 (158 mg, 399.54 μmol) in TFA (5.0 mL), Triflic acid (1.20 g, 7.99 mmol, 701.33 μL) was added drop wise at 0° C. and stirred for 16 hr at RT. After completion of reaction (as monitored by LCMS), the reaction mixture was evaporated and quenched with saturated sodium bicarbonate solution. Aqueous phase was extracted with ethyl acetate (3×25 mL) and washed with water followed by brine. The organic part was separated, dried over sodium sulphate and concentrated to afford crude 5-(4,6-dimethylpyrimidin-2-yl)-1H-benzo[cd]indol-2-one 6 (67 mg, 238.50 μmol, 57% yield) as a brown solid which was used in the next step without purification. LC-MS (ES⁺): m/z 276.2 [M+H]⁺.

Step 5: To a cooled solution of 5-(4,6-dimethylpyrimidin-2-yl)-1H-benzo[cd]indol-2-one 6 (67.06 mg, 243.60 μmol) in dry THE (5 mL), sodium hydride (60% dispersion in mineral oil) (93.34 mg, 2.44 mmol) was added portion wise, maintaining the temp <5° C. Once the addition is over, the resultant mixture was stirred for 15 minutes at RT. Then the reaction mixture was again cooled to 0° C. and 3-bromopiperidine-2,6-dione 7 (233.87 mg, 1.22 mmol) was added to it portion wise. After complete addition, resulting solution was heated at 70° C. 1 hr. After complete consumption of the starting material, the reaction mixture was cooled to 0° C. and quenched with the addition of ice-cold water. The aqueous layer was extracted with ethyl acetate (3×50 mL).

The combined organics was separated, dried over anhydrous sodium sulfate and concentrated under reduced pressure to afford crude residue which was purified by PREP-TLC to afford 3-[5-(4,6-dimethylpyrimidin-2-yl)-2-oxo-benzo[cd]indol-1-yl]piperidine-2,6-dione Compound 87 (20 mg, 51.76 μmol, 21% yield) as yellow solid. ¹H NMR (400 MHz, DMSO-d₆) δ 11.15 (s, 1H), 8.68 (dd, J₁=8.8 Hz, J₂=7.44 Hz, 2H), 8.21 (d, J=7.36 Hz, 1H), 7.59 (t, J=7.32 Hz, 1H), 7.36 (s, 1H), 7.20 (d, J=7.08 Hz, 1H), 5.49 (dd, J₁=1.334 Hz, J₂=5.08 Hz, 1H), 2.95-2.91 (br m, 1H), 2.8-2.77 (m, 2H), 258 (s, 6H), 2.13-2.08 (m, 1H). LC-MS (ES⁺): m/z 387.3 [M+H]⁺.

### Example 73. 3-(10-chloro-3-oxo-2,11-diazatricyclo[6.3.1.04, 12]dodeca-1(11),4(12),5,7,9-pentaen-2-yl)piperidine-2,6-dione (Compound 88)

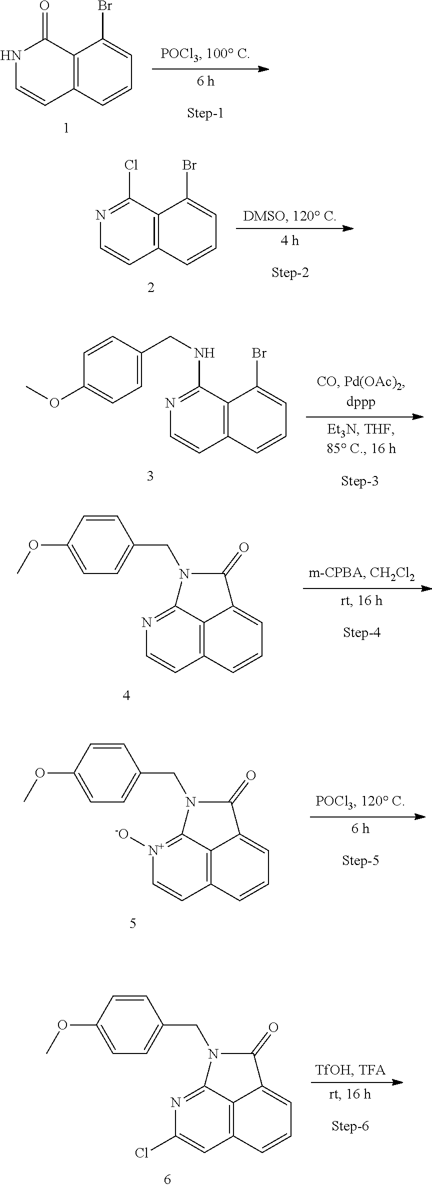

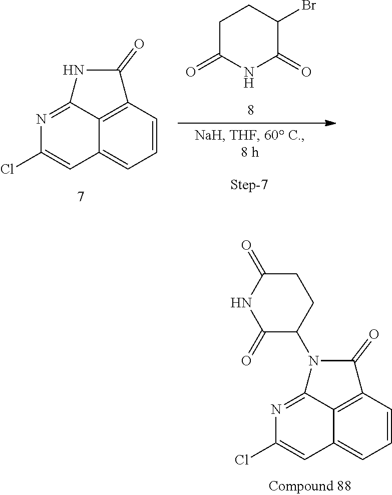 [SMILES: O=C1Nc2nc(Cl)cc3cccc1c23 ; O=C1CCC(Br)C(=O)N1 ; O=C1CCC(N2C(=O)c3cccc4cc(Cl)nc2c34)C(=O)N1]

Step 1: 8-Bromo-2H-isoquinolin-1-one 1 (2 g, 8.93 mmol) was taken in phosphorus oxychloride (20 g, 130.44 mmol) and the resulting mixture was heated at 100° C. for 6 h. The reaction mixture was cooled to room temperature and concentrated under reduced pressure to give the crude product. The crude product was purified by flash chromatography (silica gel, 230-400 mesh) using 0-100% ethyl acetate in petroleum ether while the desired compound eluted at 25% ethyl acetate in petroleum ether to give 8-bromo-1-chloro-isoquinoline 2 (1.5 g, 6.13 mmol, 69% yield) as an off-white solid. LC-MS (ES⁺): m/z 242.0 [M+H]⁺.

Step 2: To a solution of 8-bromo-1-chloro-isoquinoline 2 (1.4 g, 5.77 mmol) in DMSO (4.37 mL), was added (4-methoxyphenyl)methanamine (1.19 g, 8.66 mmol, 1.13 mL). The resulting mixture was heated at 120° C. for 4 h. The reaction mixture was treated with water (70 mL) and extracted with ethyl acetate (2×150 mL). The combined organics were washed with brine solution (30 mL) and dried over anhydrous sodium sulfate. The solution was filtered, and the filtrate was concentrated under reduced pressure to give the crude product. The crude product was purified by flash chromatography (silica-gel, 230-400 mesh) using 0-100% ethyl acetate in petroleum ether while the desired product eluted with 40-50% to give 8-bromo-N-[(4-methoxyphenyl)methyl]isoquinolin-1-amine 3 (1.8 g, 4.72 mmol, 82% yield) as an yellow gummy solid. LC-MS (ES⁺): m/z 343.2 [M+H]⁺.

Step 3: To a solution of 8-bromo-N-[(4-methoxyphenyl)methyl]isoquinolin-1-amine 3 (1 g, 2.91 mmol) in THE (20 mL), were added palladium(II) acetate (327.07 mg, 1.46 mmol), 1,3-bis(diphenylphosphino)propane (360.51 mg, 874.08 μmol) and triethylamine (884.48 mg, 8.74 mmol, 1.22 mL). The resulting mixture was heated at 85° C. in an atmosphere of carbon monoxide (5.5 kg/cm²) for 16 h. The reaction mixture was filtered through a pad of celite and the filtrate was concentrated under reduced pressure to give the crude product. The crude product was purified by flash chromatography (silica gel, 230-400 mesh) using 0-100% ethyl acetate in petroleum ether while the desired compound eluted at 20-30% ethyl acetate in petroleum ether to give 2-[(4-methoxyphenyl)methyl]-2,11-diazatricyclo[6.3.1.04, 12]dodeca-1(11),4,6,8(12),9-pentaen-3-one 4 (800 mg, 2.70 mmol, 93% yield) as an yellow solid. LC-MS (ES⁺): m/z 291.1 [M+H]⁺.

Step 4: To a solution of 2-[(4-methoxyphenyl)methyl]-2,11-diazatricyclo[6.3.1.04, 12]dodeca-1(11),4,6,8(12),9-pentaen-3-one 4 (1.4 g, 4.82 mmol) in dichloromethane (30 mL), was added m-CPBA (2.50 g, 14.47 mmol). The resulting mixture was stirred at room temperature for 16 h. The reaction mixture was treated with 10% aqueous sodium bicarbonate solution (70 mL) and extracted with dichloromethane (2×100 mL). The combined organics were washed with brine solution (50 mL) and dried over anhydrous sodium sulfate. The solution was filtered and concentrated under reduced pressure to give the crude product. The crude product was purified by flash chromatography (silica gel, 230-400 mesh) eluted at 0-20% methanol in dichloromethane to give 2-[(4-methoxyphenyl)methyl]-11-oxido-2-aza-11-azoniatricyclo[6.3.1.04, 12]dodeca-1(11),4,6,8(12),9-pentaen-3-one 5 (960 mg, 2.92 mmol, 61% yield) as a yellow solid. LC-MS (ES⁺): m/z 307.1 [M+H]⁺.

Step 5: 2-[(4-Methoxyphenyl)methyl]-11-oxido-2-aza-11-azoniatricyclo[6.3.1.04, 12]dodeca-1(11),4,6,8(12),9-pentaen-3-one 5 (960 mg, 3.13 mmol) was taken in phosphorus oxychloride (10 g, 65.22 mmol) and the resulting mixture was heated at 120° C. for 6 h. The reaction mixture was cooled to room temperature and concentrated under reduced pressure to give the crude product. The crude product was purified by flash chromatography (silica gel, 230-400 mesh) eluted with 25% ethyl acetate in petroleum ether to give 10-chloro-2-[(4-methoxyphenyl)methyl]-2,11-diazatricyclo[6.3.1.04, 12]dodeca-1(11),4,6,8(12),9-pentaen-3-one 6 (380 mg, 926.60 μmol, 30% yield) as a yellow solid. LC-MS (ES⁺): m/z 325.1 [M+H]⁺.

Step 6: To a solution of 10-chloro-2-[(4-methoxyphenyl)methyl]-2,11-diazatricyclo[6.3.1.04, 12]dodeca-1(11),4(12),5,7,9-pentaen-3-one 6 (280 mg, 862.17 μmol) in TFA (14.75 g, 129.33 mmol, 9.96 mL), was added triflic acid (1.71 g, 11.38 mmol, 1.0 mL). The resulting mixture was stirred at room temperature for 16 h. The reaction mixture was concentrated under reduced pressure to give the residue which was washed with cold water (15 mL) and extracted with dichloromethane (2×30 mL). The combined organics were washed with brine solution (20 mL) and dried over anhydrous sodium sulfate. The solution was filtered and concentrated under reduced pressure to give the crude product. The crude product was triturated with methyl tert-butyl ether, the precipitated solid was filtered, washed with methyl tert-butyl ether and dried under vacuum to give 10-chloro-2,11-diazatricyclo[6.3.1.04, 12]dodeca-1(11),4(12),5,7,9-pentaen-3-one 7 (230 mg, 838.56 μmol, 97% yield) as a yellow solid. LC-MS (ES⁺): m/z 205.2 [M+H]⁺.

Step 7: To a solution of 10-chloro-2,11-diazatricyclo[6.3.1.04, 12]dodeca-1(11),4(12),5,7,9-pentaen-3-one 7 (100 mg, 488.73 μmol) in THE (4 mL), cooled to 0° C., was added sodium hydride (60% dispersion in mineral oil, 67.42 mg, 2.93 mmol). The resulting mixture was stirred at room temperature for 30 min. The mixture was cooled to 0° C., was added 3-bromopiperidine-2,6-dione 8 (234.60 mg, 1.22 mmol) in THE (2 mL) drop-wise. The resulting mixture was heated at 60° C. for 8 h. The reaction mixture was cooled to 0° C., treated with saturated ammonium chloride solution and extracted with ethyl acetate (2×10 mL). The combined organics were washed with brine solution (5 mL) and dried over anhydrous sodium sulfate. The solution was filtered and concentrated under reduced pressure to give the crude product. The crude product was purified by flash chromatography (silica gel, 230-400 mesh) eluted with 60-70% ethyl acetate in petroleum ether to give the product with 66% pure. The compound was again purified by reverse phase purification [Column: X select C18 (250×19) mm, 5 microns, Mobile phase: A: 0.1% Ammonium acetate in water, B: Acetonitrile] and the fractions containing the product was lyophilized to give 3-(10-chloro-3-oxo-2,11-diazatricyclo[6.3.1.04, 12]dodeca-1(11),4(12),5,7,9-pentaen-2-yl)piperidine-2,6-dione Compound 88 (7.0 mg, 14.59 μmol, 3% yield) as a yellow solid. LC-MS (ES⁺): m/z 316.0 [M+H]⁺. ¹H NMR (400 MHz, DMSO-d₆): δ 11.10 (s, 1H), 8.33 (s, 1H), 8.32-8.26 (m, 2H), 8.19-8.16 (m, 1H), 5.46-5.41 (m, 1H), 2.99-2.94 (m, 1H), 2.84-2.69 (m, 1H), 2.68-2.64 (m, 1H), 2.18-2.14 (m, 1H).

### Example 74. 3-(10-methoxy-3-oxo-2,11-diazatricyclo[6.3.1.04, 12]dodeca-1(12),4,6,8,10-pentaen-2-yl)piperidine-2,6-dione (Compound 89)

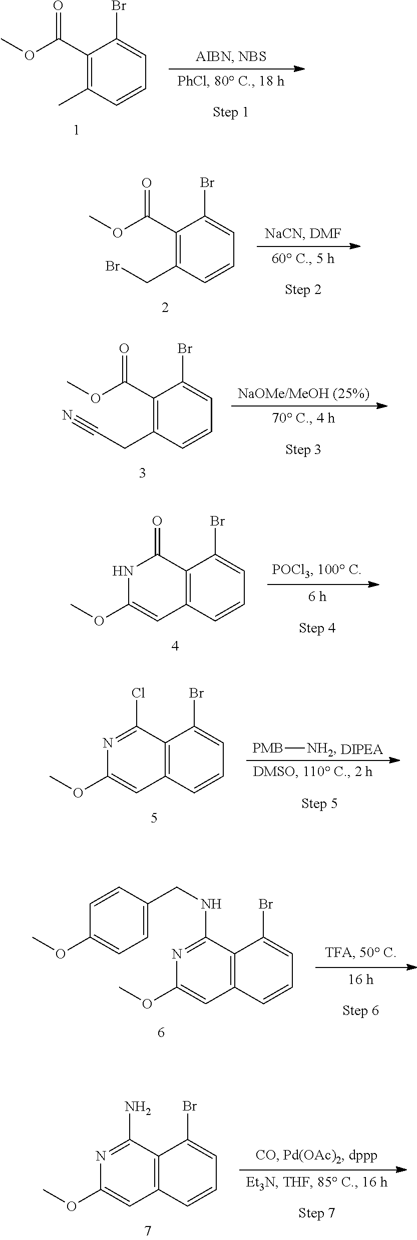

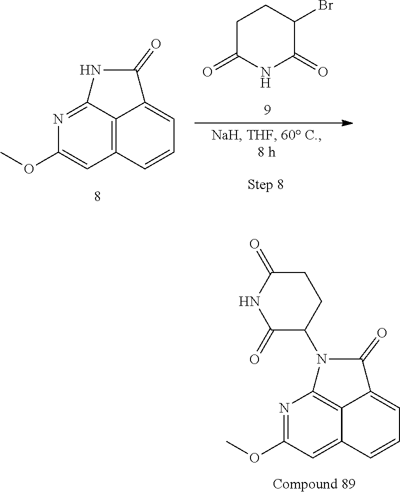 [SMILES: COc1cc2cccc3c2c(n1)NC3=O ; O=C1CCC(Br)C(=O)N1 ; COc1cc2cccc3c2c(n1)N(C1CCC(=O)NC1=O)C3=O]

Step 1: To a solution of methyl 2-bromo-6-methyl-benzoate 1 (2.5 g, 10.91 mmol) in chlorobenzene (40.00 mL), were added azobisisobutyronitrile (8.96 mg, 54.57 μmol), N-bromosuccinimide (1.94 g, 10.91 mmol, 925.84 μL). The resulting mixture was heated at 80° C. for 18 h. The reaction mixture was cooled to room temperature and concentrated under reduced pressure to give the crude product. The crude product was purified by flash chromatography (silica gel, 230-400 mesh) eluted with 15-20% ethyl acetate in petroleum ether to give methyl 2-bromo-6-(bromomethyl)benzoate 2 (3 g, 7.66 mmol, 70% yield) as a brown gum. ¹H NMR (400 MHz, CDCl3): δ 7.58 (d, J=8.4 Hz, 1H), 7.41 (d, J=7.6 Hz, 1H), 7.17 (d, J=4.4 Hz, 1H), 4.51 (s, 3H), 3.97 (s, 2H) ppm.

Step 2: To a solution of methyl 2-bromo-6-(bromomethyl)benzoate 2 (3.0 g, 7.66 mmol) in N,N-imethylformamide (15 mL), was added sodium cyanide (750.41 mg, 15.31 mmol). The resulting mixture was heated at 60° C. for 5 h. The reaction mixture was cooled to room temperature, treated with water (50 mL) and extracted with ethyl acetate (3×50 mL). The combined organics were washed with brine solution (50 mL) and dried over anhydrous sodium sulfate. The solution was filtered and concentrated under reduced pressure to give the crude product. The crude product was purified by flash chromatography (silica gel, 230-400 mesh) eluted with 30-40% ethyl acetate in petroleum ether to give methyl 2-bromo-6-(cyanomethyl)benzoate 3 (1.2 g, 4.01 mmol, 52% yield) as a brown gum. ¹H NMR (400 MHz, DMSO-d₆): δ 7.75 (d, J=8.0 Hz, 1H), 7.55 (d, J=8.4 Hz, 1H), 7.50 (d, J=7.6 Hz, 1H), 4.50 (s, 3H), 3.97 (s, 2H) ppm.

Step 3: To a solution of methyl 2-bromo-6-(cyanomethyl)benzoate 3 (1.2 g, 3.20 mmol) in methanol (9.41 mL), was added sodium methoxide (25% in methanol, 3.46 g, 16.01 mmol, 3.57 mL). The resulting mixture was heated at 70° C. for 4 h. The reaction mixture was cooled to room temperature and acidified using 1N HCl. The precipitated solid was filtered, washed with water and dried under vacuum to give 8-bromo-3-methoxy-2H-isoquinolin-1-one 4 (1 g, 3.38 mmol, 106% yield) as a yellow solid. LC-MS (ES⁺): m/z 254.6 [M+H]⁺.

Step 4: 8-Bromo-3-methoxy-2H-isoquinolin-1-one 4 (500 mg, 1.97 mmol) was taken in phosphoryl trichloride (16.40 g, 106.96 mmol, 10 mL) and the resulting solution was heated at 100° C. for 6 h. The reaction mixture was concentrated under reduced pressure to give the residue which was treated with cold water (30 mL). The precipitated solid was filtered, washed with water (10 mL) and dried under vacuum to give 8-bromo-1-chloro-3-methoxy-isoquinoline 5 (510 mg, 1.82 mmol, 92% yield) as an off-white solid. LC-MS (ES⁺): m/z 272.1 [M+H]⁺.

Step 5: To a solution of 8-bromo-1-chloro-3-methoxy-isoquinoline 5 (200 mg, 733.88 μmol) and (4-methoxyphenyl)methanamine (151.01 mg, 1.10 mmol, 143.82 μL) in DMSO (2 mL), was added DIPEA (284.55 mg, 2.20 mmol, 383.48 μL). The resulting mixture was heated at 110° C. for 2 h. The reaction mixture was treated with cold water (10 mL) and extracted with ethyl acetate (2×10 mL). The combined organics were washed with brine solution (5 mL) and dried over anhydrous sodium sulfate. The solution was filtered and concentrated under reduced pressure to give the crude product. The crude product was purified by flash chromatography (silica gel, 230-400 mesh) eluted with 0-10% ethyl acetate in petroleum ether to give 8-bromo-3-methoxy-N-[(4-methoxyphenyl)methyl]isoquinolin-1-amine 6 (200 mg, 515.00 μmol, 70% yield) as a yellow solid. LC-MS (ES⁺): m/z 373.1 [M+H]⁺.

Step 6: 8-Bromo-3-methoxy-N-[(4-methoxyphenyl)methyl]isoquinolin-1-amine 6 (200 mg, 535.84 μmol) was taken in TFA (2.96 g, 25.96 mmol, 2 mL) and the resulting mixture was heated at 50° C. for 16 h. The reaction mixture was concentrated under reduced pressure to give the residue which was triturated with methyl tert-butyl ether, washed with methyl tert-butyl ether and dried under vacuum to give 8-bromo-3-methoxy-isoquinolin-1-amine trifluoroacetate 7 (150 mg, 325.93 mol, 61% yield) as a brown solid. LC-MS (ES⁺): m/z 255.0 [M+H]⁺. The crude product was taken to next step without purification.

Step 7: To a solution of 8-bromo-3-methoxy-isoquinolin-1-amine 7 (150 mg, 468.20 μmol) in THF (5 mL) were added palladium(II) acetate (52.56 mg, 234.10 μmol), 1,3-bis(diphenylphosphino)propane (57.93 mg, 140.46 μmol) and triethylamine (236.89 mg, 2.34 mmol, 326.29 μL). The reaction mixture was heated at 85° C. in an atmosphere of carbon monoxide (5.0 kg/cm2) for 16 h. The reaction mixture was filtered through a pad of celite, and the filtrate was concentrated under reduced pressure to give the crude product. The crude product was purified by flash chromatography (silica gel, 230-400 mesh) eluted with 20-30% ethyl acetate in petroleum ether to give 10-methoxy-2,11-diazatricyclo[6.3.1.04, 12]dodeca-1(11),4,6,8(12),9-pentaen-3-one 8 (65 mg, 305.66 μmol, 65% yield) as a yellow solid. LC-MS (ES⁺): m/z 201.0 [M+H]⁺.

Step 8: To a solution of 10-methoxy-2,11-diazatricyclo[6.3.1.04, 12]dodeca-1(12),4,6,8,10-pentaen-3-one 8 (65 mg, 324.69 μmol) in THE (3 mL), cooled to 0° C., was added sodium hydride (60% dispersion in mineral oil, 74.65 mg, 1.95 mmol). The resulting mixture was stirred at room temperature for 30 min. The mixture was cooled to 0° C., was added 3-bromopiperidine-2,6-dione 9 (62.34 mg, 324.69 μmol) in THE (2 mL) drop-wise. The reaction mixture was heated at 60° C. for 8 h. The reaction mixture was cooled to 0° C., treated with saturated ammonium chloride and extracted with ethyl acetate (2×10 mL). The combined organics were washed with brine solution (5 mL) and dried over anhydrous sodium sulfate. The solution was filtered and concentrated under reduced pressure to give the crude product. The crude product was purified by flash chromatography (silica gel, 230-400 mesh) eluted with 60-70% ethyl acetate in petroleum ether to give the product with 92% pure. The compound was purified again by reverse phase purification [Column: X select C18 (250×19) mm, 5 microns; Mobile phase: A: 0.1% Ammonium acetate in water, B: Acetonitrile] and lyophilized to afford 3-(10-methoxy-3-oxo-2,11-diazatricyclo[6.3.1.04, 12]dodeca-1(12),4,6,8,10-pentaen-2-yl)piperidine-2,6-dione Compound 89 (8.0 mg, 25.60 μmol, 8% yield) as a yellow solid. LC-MS (ES⁺): m/z 312.0 [M+H]⁺. ¹H NMR (400 MHz, DMSO-d₆): δ 11.16 (s, 1H), 8.08 (dd, J=7.2, 1.2 Hz, 1H), 7.96-7.90 (m, 2H), 6.80 (s, 1H), 5.41-5.36 (m, 1H), 3.91 (s, 3H), 3.03-2.94 (m, 1H), 2.90-2.83 (m, 1H), 2.69-2.65 (m, 1H), 2.39-2.33 (m, 1H) ppm.

### Example 75. 3-[5-(1-methylazetidin-3-yl)-2-oxo-benzo[cd]indol-1-yl]piperidine-2,6-dione (Compound 90)

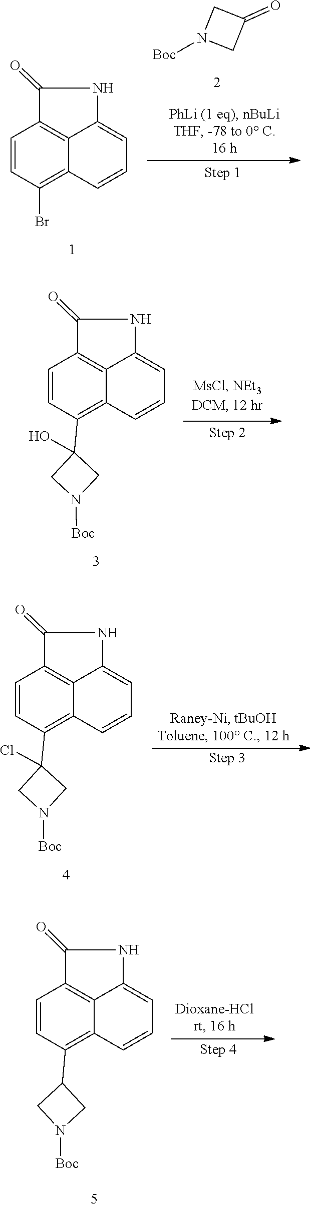 [SMILES: CC(C)(C)OC(=O)N1CC(=O)C1 ; O=C1Nc2cccc3c(Br)ccc1c23 ; CC(C)(C)OC(=O)N1CC(O)(c2ccc3c4c(cccc24)NC3=O)C1 ; CC(C)(C)OC(=O)N1CC(Cl)(c2ccc3c4c(cccc24)NC3=O)C1 ; CC(C)(C)OC(=O)N1CC(c2ccc3c4c(cccc24)NC3=O)C1]

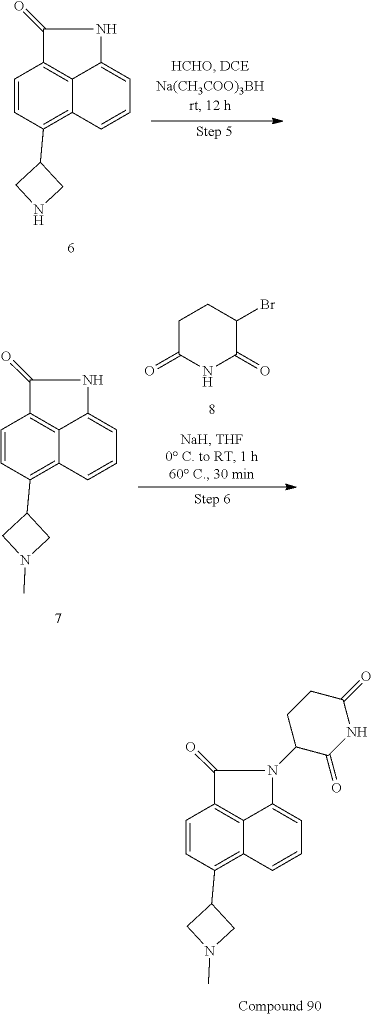 [SMILES: O=C1Nc2cccc3c(C4CNC4)ccc1c23 ; CN1CC(c2ccc3c4c(cccc24)NC3=O)C1 ; O=C1CCC(Br)C(=O)N1 ; CN1CC(c2ccc3c4c(cccc24)N(C2CCC(=O)NC2=O)C3=O)C1]

Step 1: To the stirred solution of 5-bromo-1H-benzo[cd]indol-2-one 1 (1 g, 4.03 mmol) in dry THF (10.0 mL) was added phenyllithium in di-n-butyl ether (1.9 M, 2.12 mL) at −78° C. under argon atmosphere and the reaction was stirred at the same temperature for 30 minutes followed by the addition of butyllithium (1.62 M, 2.74 mL) at −78° C. After complete addition, the temperature was allowed to increase to −40° C. and the reaction mixture was stirred at the same temperature for 30 minutes. Subsequently, a solution of tert-butyl 3-oxoazetidine-1-carboxylate 2 (690.09 mg, 4.03 mmol) in THE (10.0 mL) was added at −78° C. and then the reaction mixture was allowed to warm to room temperature and stirred for another 16 hours at this temperature. After completion of the reaction, the reaction mixture was quenched with ammonium chloride solution and diluted with ethyl acetate (100 mL). The combined organic phase was washed with water and separated, dried over anhydrous sodium sulfate, and evaporated under reduced pressure. The crude product was purified by flash chromatography using 0-5% MeOH-DCM to afford tert-butyl 3-hydroxy-3-(2-oxo-1H-benzo[cd]indol-5-yl)azetidine-1-carboxylate 3 (390.0 mg, 1.05 mmol, 26% yield) as brown solid. LC-MS (ES⁺): m/z 341.4 [M+H]⁺.

Step 2: To a Solution of tert-butyl 3-hydroxy-3-(2-oxo-1H-benzo[cd]indol-5-yl)azetidine-1-carboxylate 3 (360 mg, 1.06 mmol) in HPLC grade CHCl₃ (10.0 mL) was added triethylamine (428.10 mg, 4.23 mmol, 589.67 μL) at 0° C. and stirred for 10 minutes followed by the addition of methanesulfonyl chloride (484.63 mg, 4.23 mmol, 327.45 μL). The resulting solution was then heated at 80° C. for 16 hours. After completion of the reaction, the reaction mixture was diluted with ethyl acetate (100 mL) and washed with sodium bicarbonate solution/brine. The organic layer was separated, dried over anhydrous sodium sulfate and evaporated under reduced pressure to obtain crude tert-butyl 3-chloro-3-(2-oxo-1H-benzo[cd]indol-5-yl)azetidine-1-carboxylate 4 (428 mg, 644.11 μmol, 61% yield) as a brown solid, which was used in the next step without purification. LC-MS (ES⁺): m/z 359.3 [M+H]⁺.

Step 3: To a suspension of tert-butyl 3-chloro-3-(2-oxo-1H-benzo[cd]indol-5-yl)azetidine-1-carboxylate 4 (428 mg, 1.19 mmol) in tert Butanol (4 mL) and toluene (4 mL) was added Raney Nickel 2800, slurry, in H₂O, active catalyst (1.02 g, 11.93 mmol) and the reaction mixture was degassed for 10 minutes prior to heating at 100° C. for 12 hr. After completion of reaction (as evidenced from TLC), the reaction mixture was cooled to room temperature, filtered through a pad of celite, washed with 10% MeOH/DCM. The filtrate was then concentrated under reduced pressure to afford tert-butyl 3-(2-oxo-1H-benzo[cd]indol-5-yl)azetidine-1-carboxylate 5 (370 mg, 489.34 μmol, 41% yield) which was used directly in the next step without purification. LC-MS (ES⁺): m/z 325.4 [M+H]⁺.

Step 4: To the stirred solution of tert-butyl 3-(2-oxo-1H-benzo[cd]indol-5-yl)azetidine-1-carboxylate 5 (370.0 mg, 1.14 mmol) in 1,4-dioxane (3 mL), 4 M dioxane-HCl (1.14 mmol, 10 mL) was added at 0° C. and the reaction mixture was stirred for 16 hours at rt. After completion of the reaction, the volatiles were removed under reduced pressure to obtain a solid which was washed with ether and pentane to afford 5-(1-chloroazetidin-3-yl)-1H-benzo[cd]indol-2-one hydrochloride 6 (290.0 mg, 478.16 μmol, 42% yield) as a yellow solid which was used in the next step without purification. LC-MS (ES⁺): m/z 225.4 [M+H]⁺.

Step 5: To a well stirred solution of 5-(1-chloroazetidin-3-yl)-1H-benzo[cd]indol-2-one hydrochloride 6 (90.0 mg, 1.11 mmol) in HPLC grade DCM-MeOH (5:2, v/v, 7 mL) was added triethylamine (112.55 mg, 1.11 mmol, 155.03 μL) and the reaction mixture was stirred at room temperature for 10 minutes followed by the addition of then formaldehyde (66.81 mg, 2.22 mmol, 61.86 μL) and acetic acid (133.59 mg, 2.22 mmol, 127.23 μL). The resulting reaction mixture was then heated at 60° C. for 3 hours. It was then warmed up to room temperature prior to the addition of sodium; triacetoxyboranuide (1.22 g, 5.75 mmol). The reaction mixture was allowed to stir at the same temperature for an additional 12 hours. After completion of the reaction, the reaction mixture was diluted with 10% MeOH in DCM (50 mL) and washed with saturated sodium bicarbonate solution followed by water/brine solution. The organic layer was separated, dried over sodium sulfate, and concentrated under reduced pressure. The crude product was purified by combi-flash column chromatography to afford 5-(1-methylazetidin-3-yl)-1H-benzo[cd]indol-2-one 7 (60.0 mg, 188.85 μmol, 17% yield). LC-MS (ES⁺): m/z 239.0 [M+H]⁺.

Step 6: To a cooled solution of 5-(1-methylazetidin-3-yl)-1H-benzo[cd]indol-2-one 7 (160.0 mg, 671.47 μmol) in dry THE (5 mL) was added NaH (15.44 mg, 671.47 μmol) portion wise, maintaining the temp <5° C. Once the addition is over, the resulting mixture was stirred for 15 minutes at room temperature. Then the reaction mixture was cooled to 0° C. and 3-bromopiperidine-2,6-dione 8 (128.93 mg, 671.47 μmol) was added portion wise and the resulting solution was heated at 70° C. 1 hour before being cooled to 0° C. and quenched with the addition of ice-cold water (5 mL). The aqueous layer was extracted with ethyl acetate (3×50 mL) and the combined organic layer was separated, dried over anhydrous sodium sulfate, and concentrated under reduced pressure. The crude product was purified by PREP-TLC to afford 3-[5-(1-methylazetidin-3-yl)-2-oxo-benzo[cd]indol-1-yl]piperidine-2,6-dione Compound 90 (5.8 mg, 15.85 μmol, 2% yield). ¹H NMR (400 MHz, DMSO-d₆) δ 11.11 (s, 1H), 8.07 (d, J=7.28 Hz, 1H), 7.79 (d, J=7.12 Hz, 1H), 7.62 (d, J=8.48 Hz, 1H), 7.51 (t, J=7.32 Hz, 1H), 7.14 (d, J=7.16 Hz, 1H), 5.44-5.42 (m, 1H), 4.38-4.35 (m, 1H), 3.91 (m, 2H), 3.43 (m, 2H), 2.94-2.91 (m, 1H), 2.77-2.62 (m, 2H), 2.32 (s, 3H), 2.10-2.07 (m, 1H). LC-MS (ES⁺): m/z 350.1 [M+H]⁺.

### Example 76. 3-(8-(1-(3-(morpholinosulfonyl)benzyl)piperidin-4-yl)-5-oxopyrrolo[2,3,4-de]quinolin-4(5H)-yl)piperidine-2,6-dione (Compound 91)

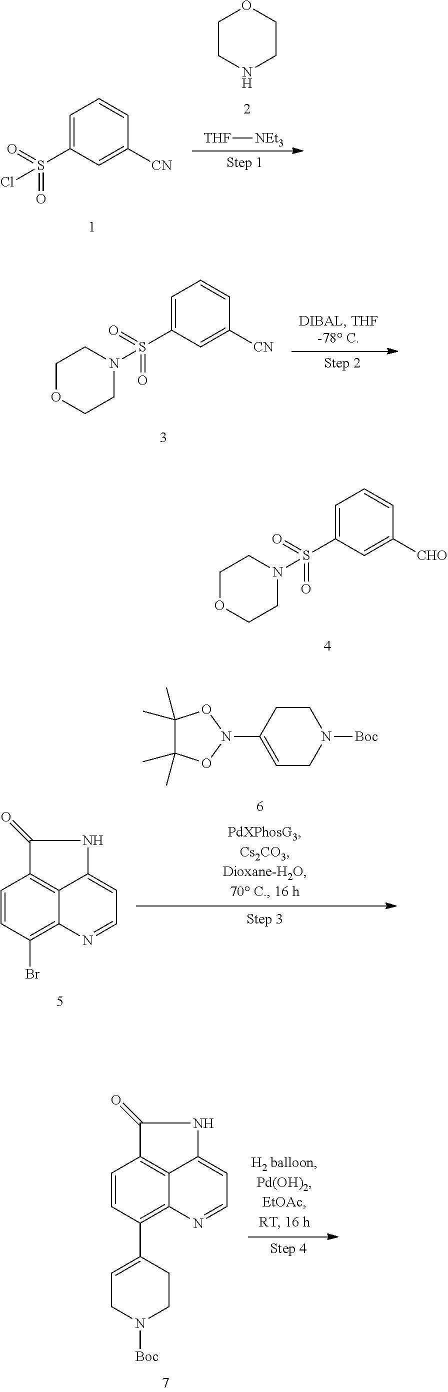

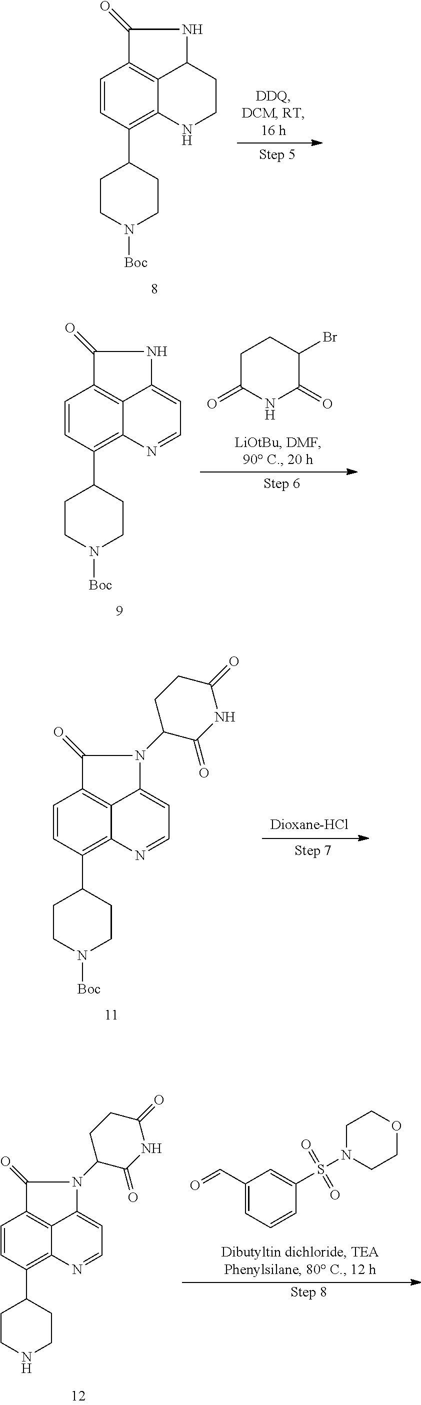

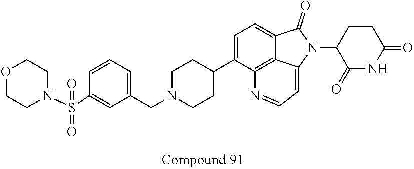 [SMILES: O=C1CCC(N2C(=O)c3ccc(C4CCN(Cc5cccc(S(=O)(=O)N6CCOCC6)c5)CC4)c4nccc2c34)C(=O)N1]

Step 1: In a flame dried 100 mL round-bottom flask under nitrogen atmosphere, 3-cyanobenzenesulfonyl chloride 1 (1.8 g, 8.93 mmol) was dissolved in dry THE (20 mL) and cooled down to 0° C. To this solution, triethylamine (1.81 g, 17.85 mmol, 2.49 mL) and morpholine 2 (933.29 mg, 10.71 mmol, 937.04 μL) were added under inert atmosphere. The resulting reaction mixture was warmed up to room temperature and stirred for 12 hours. After completion of the reaction, the volatiles were removed under vacuum and the crude product was purified by flash column chromatography to afford 3-morpholinosulfonylbenzonitrile 3 (1.85 g, 5.97 mmol, 67% yield). LC-MS (ES⁺): m/z 253.3 [M+H]⁺.

Step 2: To the stirred solution of 3-morpholinosulfonylbenzonitrile 3 (500 mg, 1.98 mmol) in dry THF (200 mL) was added DIBAL-H (2.03 g, 3.57 mmol, 2.89 mL) dropwise at 0° C. and stirred for another 16 hours at room temperature. After completion of the reaction, the reaction mixture was diluted with ethyl acetate (100 mL) and quenched with saturated solution of rochelle's salt. The resulting turbid solution was stirred for 2 hours until a clear aqueous-organic layer separation was observed. The organic layer was separated, dried over anhydrous sodium sulfate, and evaporated under reduced pressure to obtain the crude compound which was purified by flash chromatography using 0-10% ethyl acetate-DCM to afford 3-morpholinosulfonylbenzaldehyde 4 (200 mg, 783.42 μmol, 40% yield) as colorless gum. LC-MS (ES⁺): m/z 256.1 [M+H]⁺.

Step 3: To a well degassed solution of 6-bromo-10,11-diazatricyclododeca-,2(5),3(10),4(7),6(8)-pentaen-9-one 5 (500 mg, 2.01 mmol), tert-butyl 4-(4,4,5,5-tetramethyl-1,3,2-dioxaborolan-2-yl)-3,6-dihydro-2H-pyridine-1-carboxylate 6 (744.89 mg, 2.41 mmol) in dioxane (6 mL)-water (1.5 mL), cesium carbonate (1.64 g, 5.02 mmol) was added followed by XPhos-Pd-G3 (254.89 mg, 301.13 μmol). The resulting reaction mixture was heated at 90° C. for 16 hours. After completion of the reaction, the reaction mixture was diluted with ethyl acetate (25 mL), filtered through a pad of celite and washed with ethyl acetate. The combined organic layers were washed with water, brine, dried over anhydrous sodium sulfate, filtered and concentrated under reduced pressure. The crude product was purified by column chromatography to afford tert-butyl 4-(17-oxo-20,21-diazatricyclododeca-3,5(13),6(20),11(14),12(15)-pentaen-12-yl)-3,6-dihydro-2H-pyridine-1-carboxylate 7 (300 mg, 700.06 μmol, 35% yield). LC-MS (ES⁺): m/z 352.0 [M+H]⁺.

Step 4: To a degassed solution of tert-butyl 4-(17-oxo-20,21-diazatricyclododeca-3,5(13),6(20),11(14),12(15)-pentaen-12-yl)-3,6-dihydro-2H-pyridine-1-carboxylate 7 (0.3 g, 853.73 μmol) in ethyl acetate (15 mL), dihydroxypalladium (0.27 g, 1.92 mmol) was added and resulting reaction mixture was hydrogenated with H2 balloon at room temperature for 16 hours. After complete consumption of the starting material, the reaction mixture was filtered through a celite bed and washed with ethyl acetate (100 mL). Filtrate was collected and concentrated under reduced pressure. The crude product was purified by flash column chromatography using ethyl acetate-hexane (10 to 50%) as eluent to afford tert-butyl 4-(3-oxo-2,9-diazatricyclo[6.3.1.0^{4, 12}]dodeca-4(12),5,7-trien-7-yl)piperidine-1-carboxylate 8 (220 mg, 615.48 mol, 72% yield);). LC-MS (ES⁺): m/z 358 [M+H]⁺.

Step 5: To a stirred solution of tert-butyl 4-(17-oxo-20,21-diazatricyclododeca-3,11(14),12-trien-11-yl)piperidine-1-carboxylate 8 (220 mg, 615.48 μmol) in HPLC grade DCM (12 mL), 2,3-dichloro-5,6-dicyano-1,4-benzoquinone (153.68 mg, 677.01 μmol) was added dropwise at 0° C. and the reaction mixture was stirred at room temperature for 16 hours. After completion of the reaction, the reaction mixture was diluted with DCM (30 mL) and washed with 1 M NaOH solution followed by brine. The organic layer was separated, dried over sodium sulfate, and concentrated under reduced pressure. The resulting crude product was purified by flash column chromatography (30% DCM-ethyl acetate) to afford tert-butyl 4-(17-oxo-20,21-diazatricyclododeca-3,5(13),6(20),11(15),12(14)-pentaen-11-yl)piperidine-1-carboxylate 9 (100 mg, 141.48 μmol, 23% yield). LC-MS (ES⁺): m/z 354 [M+H]⁺.

Step 6: To a cooled solution of tert-butyl 4-(17-oxo-20,21-diazatricyclododeca-3,5(13),6(20),11(15),12(14)-pentaen-11-yl)piperidine-1-carboxylate 9 (100 mg, 282.95 mol) in dry DMF (5 mL), lithium tert-butoxide (90.61 mg, 1.13 mmol) was added under inert atmosphere, maintaining the temp <5° C. The resulting mixture was stirred for 15 minutes at room temperature before being cooled to 0° C. and 3-bromopiperidine-2,6-dione 10 (108.66 mg, 565.91 mol) was added. The reaction was then heated at 90° C. for 16 hours. After completion of the reaction, the reaction mixture was cooled to 0° C. and quenched with the addition of saturated NH₄Cl solution. The aqueous layer was extracted with ethyl acetate (3×50 mL), and the combined organics was separated, dried over anhydrous sodium sulfate, and concentrated under reduced pressure. The crude product was purified by flash column chromatography using DCM-ethyl acetate (1:1, v/v) as eluent to afford tert-butyl 4-[28-(2,6-dioxo-3-piperidyl)-21-oxo-25,28-diazatricyclododeca-3,5(15),6(25),13(17),14(16)-pentaen-13-yl]piperidine-1-carboxylate 11 (25 mg, 38.75 μmol, 14% yield). LC-MS (ES⁺): m/z 465 [M+H]⁺.

Step 7: To a stirred solution of tert-butyl 4-[28-(2,6-dioxo-3-piperidyl)-21-oxo-25,28-diazatricyclododeca-3,5(15),6(25),13(17),14(16)-pentaen-13-yl]piperidine-1-carboxylate 11 (25 mg, 53.82 μmol) in HPLC grade dioxane (0.5 mL), dioxane-HCl (4 M, 30 μL) was added drop wise at 0° C. After complete addition, the resulting reaction mixture was stirred at room temperature for 3 hours. After complete consumption of the starting material, the volatiles were removed under reduced pressure to afford 3-[18-oxo-10-(4-piperidyl)-20,23-diazatricyclododeca-,2(12),3(20),10(14),11(13)-pentaen-23-yl]piperidine-2,6-dione hydrochloride 12 (15 mg, 37.42 mol, 70% yield) which was used in next step without any purification. LC-MS (ES⁺): m/z 365 [M+H]⁺.

Step 8: To the stirred solution of 3-[18-oxo-10-(4-piperidyl)-20,23-diazatricyclododeca-,2(12),3(20),10(14),11(13)-pentaen-23-yl]piperidine-2,6-dione hydrochloride 12 (15 mg, 37.42 μmol) in dry THF(3 mL), triethylamine (7.57 mg, 74.84 μmol, 10.43 μL) was added (pH˜7) followed by 3-morpholinosulfonylbenzaldehyde 4 (9.55 mg, 37.42 mol) and dibutyl tin dichloride (13.64 mg, 44.90 μmol, 10.03 μL). The resulting reaction mixture was heated for 1 hour at 60° C. The reaction mixture was cooled to room temperature and phenylsilane (6.07 mg, 56.13 μmol) was carefully added and then heated again at 80° C. for 12 hours. After completion of the reaction, the reaction mixture was concentrated and crude product was purified by reverse phase prep-HPLC to afford 3-[21-[1-[(3-morpholinosulfonylphenyl)methyl]-4-piperidyl]-29-oxo-31,35-diazatricyclododeca-3(21),4(22),5(23),6(31),24-pentaen-35-yl]piperidine-2,6-dione Compound 91 (2.94 mg, 4.69 mol, 13% yield). ¹H NMR (400 MHz, DMSO-d₆) δ 11.15 (s, 1H), 8.85 (d, J=4.72 Hz, 1H), 8.08 (d, J=7.28 Hz, 1H), 7.87 (d, J=7.36 Hz, 1H), 7.73 (br, 2H), 7.65-7.63 (br, 2H), 7.21 (d, J=4.76 Hz, 1H), 5.43 (dd, J1=12.92 Hz, J=5.32 Hz 1H), 3.82 (br m, 1H), 3.69 (s, 2H), 63.62 (t, J=4.24 Hz, 4H), 3.0 (m, 3H), 2.87 (t, J=4.32 Hz, 4H), 2.67-2.63 (m, 2H), 2.27-2.21 (m, 2H), 2.13-2.11 (m, 1H), 1.95-1.90 (m, 4H). LC-MS (ES⁺): m/z 604 [M+H]⁺.

Prophetic reactions to synthesize compounds of the current invention:

### Example 77. Synthesis of 3-(2-oxobenzo[cd]indol-1(2H)-yl)azepane-2,7-dione (Compound 92)

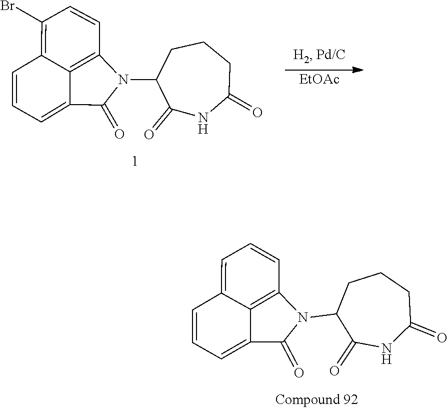 [SMILES: "EtOAc" ; O=C1CCCC(N2C(=O)c3cccc4c(Br)ccc2c34)C(=O)N1 ; O=C1CCCC(N2C(=O)c3cccc4cccc2c34)C(=O)N1]

To a stirred solution of 3-(6-bromo-2-oxobenzo[cd]indol-1(2H)-yl)azepane-2,7-dione 1 (1 eq) under a controlled nitrogen atmosphere in EtOAc (0.05 M) is added Pd/C (10% by weight). Upon addition, an H2 balloon is then sparged through the solution for 30 minutes. Stirring is continued at room temperature under an H2 atmosphere until reaction completion is evident. Upon reaction completion the mixture is filtered through a pad of celite and eluted with excess EtOAc. The filtrate is concentrated to a crude residue. Standard workup and purification procedures will furnish the product 3-(2-oxobenzo[cd]indol-1(2H)-yl)azepane-2,7-dione Compound 92.

### Example 78. Using similar conditions, the non-limiting starting materials below on the left can be converted to the corresponding products on the right

| | | Compound |
|---|---|---|
| Starting Material | Product | No. |
| 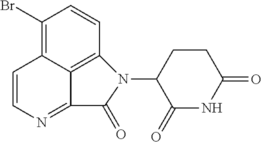 [SMILES: O=C1CCC(N2C(=O)c3nccc4c(Br)ccc2c34)C(=O)N1] | 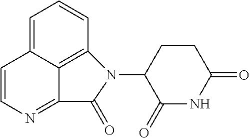 [SMILES: O=C1CCC(N2C(=O)c3nccc4cccc2c34)C(=O)N1] | Compound 93 |
| 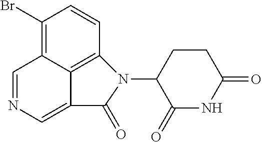 [SMILES: O=C1CCC(N2C(=O)c3cncc4c(Br)ccc2c34)C(=O)N1] | 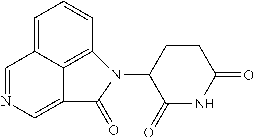 [SMILES: O=C1CCC(N2C(=O)c3cncc4cccc2c34)C(=O)N1] | Compound 94 |
| 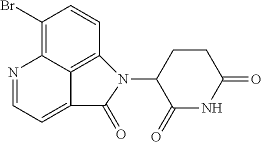 [SMILES: O=C1CCC(N2C(=O)c3ccnc4c(Br)ccc2c34)C(=O)N1] | 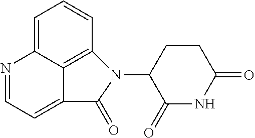 [SMILES: O=C1CCC(N2C(=O)c3ccnc4cccc2c34)C(=O)N1] | Compound 95 |
| 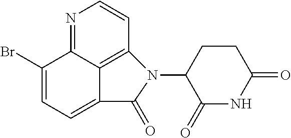 [SMILES: O=C1CCC(N2C(=O)c3ccc(Br)c4nccc2c34)C(=O)N1] | 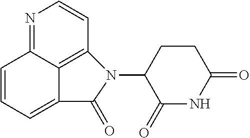 [SMILES: O=C1CCC(N2C(=O)c3cccc4nccc2c34)C(=O)N1] | Compound 96 |
| 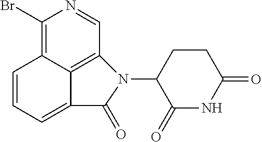 [SMILES: O=C1CCC(N2C(=O)c3cccc4c(Br)ncc2c34)C(=O)N1] | 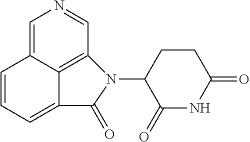 [SMILES: O=C1CCC(N2C(=O)c3cccc4cncc2c34)C(=O)N1] | Compound 97 |
| 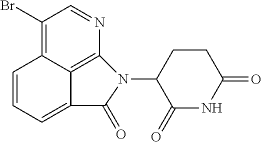 [SMILES: O=C1CCC(N2C(=O)c3cccc4c(Br)cnc2c34)C(=O)N1] | 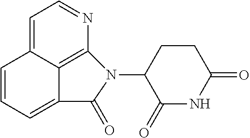 [SMILES: O=C1CCC(N2C(=O)c3cccc4ccnc2c34)C(=O)N1] | Compound 98 |

### Example 79. A non-limiting selection of the compounds of the present invention available from a common intermediate

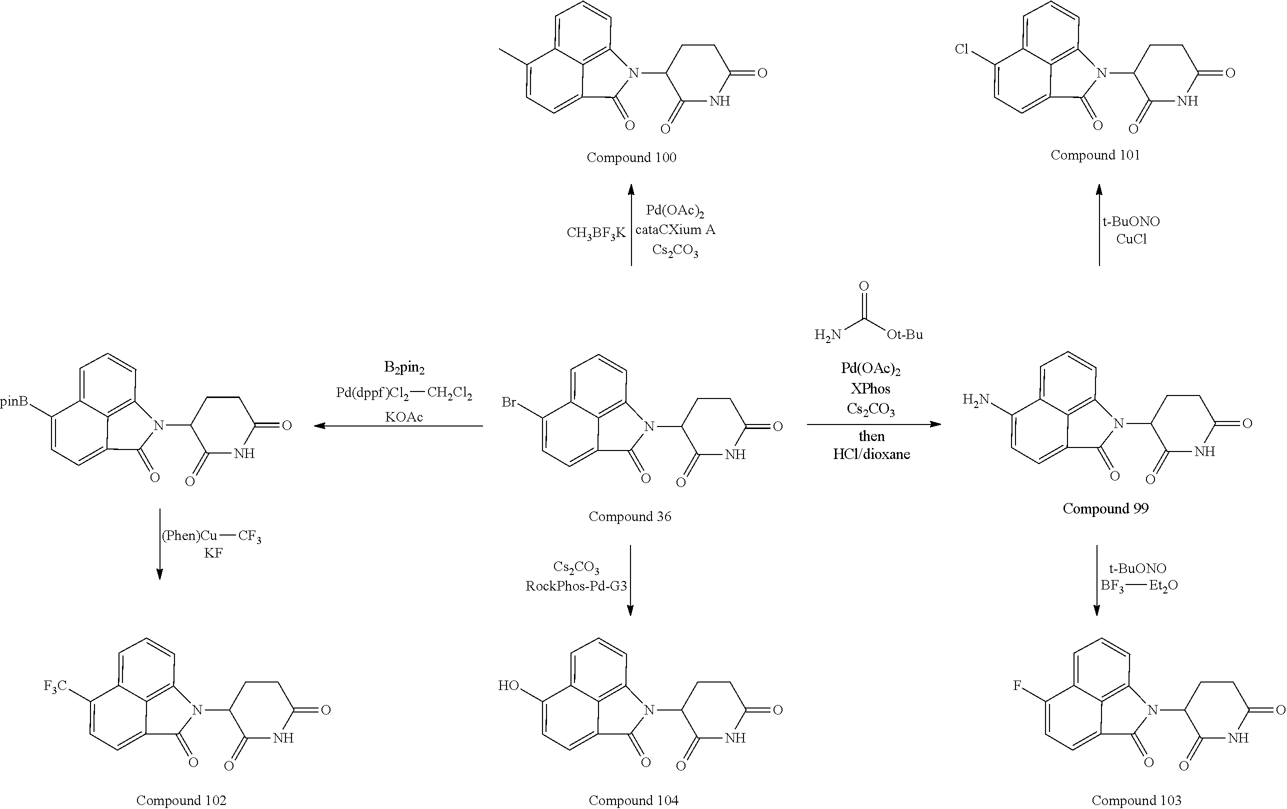

### Example 80. 3-[2-oxo-7-(4,4,5,5-tetramethyl-1,3,2-dioxaborolan-2-yl)benzo[cd]indol-1-yl]piperidine-2,6-dione (Compound 105)

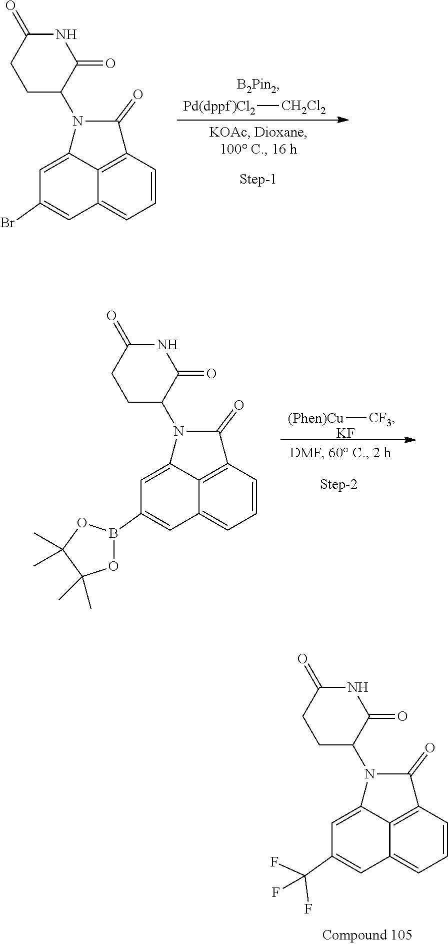

Step 1: To a solution of 3-(7-bromo-2-oxo-benzo[cd]indol-1-yl)piperidine-2,6-dione (50 mg, 139.21 μmol) and bis(pinacolato)diboron (60.10 mg, 236.65 μmol) in 1,4-dioxane (3 mL), was added potassium acetate (40.99 mg, 417.62 μmol). The contents were purged with nitrogen for 2 min. To this mixture was added [1,1′-bis(diphenylphosphino)ferrocene]dichloropalladium(II) complex with dichloromethane (11.37 mg, 13.92 μmol), and purged with nitrogen for 2 min. The contents were heated at 100° C. for 16 h. The reaction mixture was cooled to room temperature and concentrated under reduced pressure. The residue was partitioned between ethyl acetate (20 mL) and water (10 mL) and separated the organic phase. The aqueous phase was extracted with ethyl acetate (3×5 mL), the combined organics were washed with brine solution and dried over anhydrous sodium sulfate. The solution was filtered and concentrated under reduced pressure to give 3-[2-oxo-7-(4,4,5,5-tetramethyl-1,3,2-dioxaborolan-2-yl)benzo[cd]indol-1-yl]piperidine-2,6-dione (100 mg, 75.55 μmol, 54.27% yield) as a yellow solid. LCMS (ESI): m/z 407.2 [M+H]+. The crude product was taken to the next step without purification.

Step 2: To a solution of 3-[2-oxo-7-(4,4,5,5-tetramethyl-1,3,2-dioxaborolan-2-yl)benzo[cd]indol-1-yl]piperidine-2,6-dione (190 mg, 143.54 μmol) (having purity 30.69%) in N,N-dimethylformamide (10 mL), were added (1,10-Phenanthroline)(trifluoromethyl)copper(I) (67.34 mg, 215.31 μmol) and potassium fluoride (41.70 mg, 717.69 μmol). The resulting mixture was heated at 60° C. for 2 h. The reaction mixture was treated with water (10 mL) and extracted with ethyl acetate (3×15 mL). The combined organics were washed with brine solution (15 mL) and dried over anhydrous sodium sulfate. The solution was filtered and concentrated under reduced pressure to give the crude product. The crude product was purified by preparative HPLC [Column: X select C18 (250×19 mm), 5 microns; Mobile phase: A 0.1% Formic acid in water, B: Acetonitrile] and the fractions containing the product was combined and lyophilized to give 3-[2-oxo-7-(trifluoromethyl)benzo[cd]indol-1-yl]piperidine-2,6-dione Compound 105 (15 mg, 42.55 μmol, 29.65% yield) as an off-white solid. LCMS (ESI): m/z 347.0 [M−H]⁺.

| | | Compound | |
|---|---|---|---|
| Starting Material | Product | No. | LCMS |
| 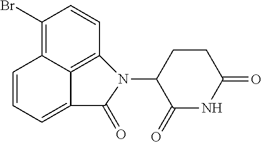 [SMILES: O=C1CCC(N2C(=O)c3cccc4c(Br)ccc2c34)C(=O)N1] | 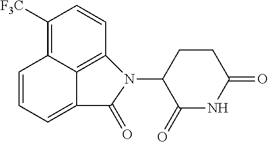 [SMILES: O=C1CCC(N2C(=O)c3cccc4c(C(F)(F)F)ccc2c34)C(=O)N1] | Compound 106 | 347.0 [M − H]⁻ |
| 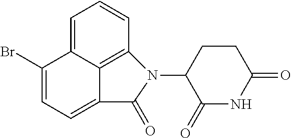 [SMILES: O=C1CCC(N2C(=O)c3ccc(Br)c4cccc2c34)C(=O)N1] | 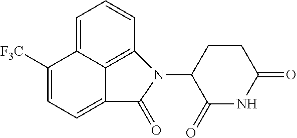 [SMILES: O=C1CCC(N2C(=O)c3ccc(C(F)(F)F)c4cccc2c34)C(=O)N1] | Compound 107 | 347.0 [M − H]⁻ |
| 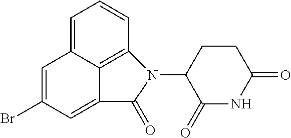 [SMILES: O=C1CCC(N2C(=O)c3cc(Br)cc4cccc2c34)C(=O)N1] | 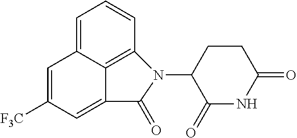 [SMILES: O=C1CCC(N2C(=O)c3cc(C(F)(F)F)cc4cccc2c34)C(=O)N1] | Compound 108 | 347.0 [M − H]⁻ |

### Example 81. 3-(7-methyl-2-oxo-benzo[cd]indol-1-yl)piperidine-2,6-dione (Compound 109)

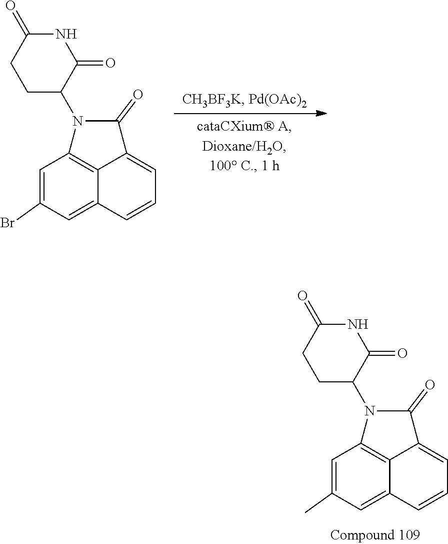 [SMILES: O=C1CCC(N2C(=O)c3cccc4cc(Br)cc2c34)C(=O)N1 ; Cc1cc2c3c(cccc3c1)C(=O)N2C1CCC(=O)NC1=O]

To a solution of 3-(7-bromo-2-oxo-benzo[cd]indol-1-yl)piperidine-2,6-dione (60 mg, 167.05 μmol) and potassium methyltrifluoroborate (101.85 mg, 835.25 μmol) in 1,4-dioxane (3 mL) and water (0.5 mL), was added cesium carbonate (163.28 mg, 501.15 μmol). The contents were purged with nitrogen for 2 min. To this mixture, were added di(1-adamantyl)-n-butylphosphine (5.99 mg, 16.71 μmol) and palladium (II) acetate (7.50 mg, 33.41 μmol) and purged with nitrogen for 2 min. The contents were heated at 100° C. for 1 h. The reaction mixture was cooled to room temperature, treated with water (4 mL) and extracted with ethyl acetate (3×10 mL). The combined organics were washed with brine solution (10 mL) and dried over anhydrous sodium sulfate. The solution was filtered and concentrated under reduced pressure to give the crude product. The crude product was purified by reverse phase C18 column [ISCO C18 column (30 g); Mobile-phase A: 0.1% HCOOH in water, Mobile phase B: Acetonitrile] and the fractions containing the compound were lyophilized to give 3-(7-methyl-2-oxo-benzo[cd]indol-1-yl)piperidine-2,6-dione Compound 109 (17 mg, 56.17 μmol, 33.63% yield) as a yellow solid. LCMS (ESI): m/z 295.0 [M+H]⁺, RT=0.85 min.

| | | Compound | |
|---|---|---|---|
| Starting Material | Product | No. | LCMS |
| 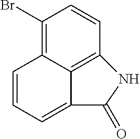 [SMILES: O=C1Nc2ccc(Br)c3cccc1c23] | 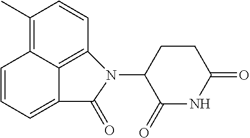 [SMILES: Cc1ccc2c3c(cccc13)C(=O)N2C1CCC(=O)NC1=O] | Compound 110 | 295.2 [M + H]⁺ |
| 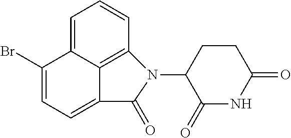 [SMILES: O=C1CCC(N2C(=O)c3ccc(Br)c4cccc2c34)C(=O)N1] | 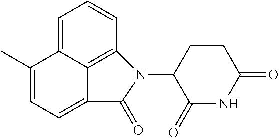 [SMILES: Cc1ccc2c3c(cccc13)N(C1CCC(=O)NC1=O)C2=O] | Compound 111 | 295.2 [M + H]⁺ |
| 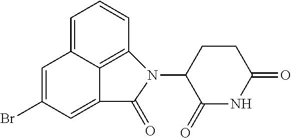 [SMILES: O=C1CCC(N2C(=O)c3cc(Br)cc4cccc2c34)C(=O)N1] | 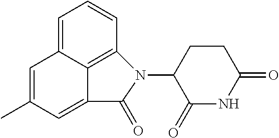 [SMILES: Cc1cc2c3c(cccc3c1)N(C1CCC(=O)NC1=O)C2=O] | Compound 112 | 295.1 [M + H]⁺ |

In the synthesis of Compound 110, addition of glutarimide was carried out after the methylation.

### Example 82. 3-(7-methyl-2-oxo-benzo[cd]indol-1-yl)piperidine-2,6-dione (Compound 113)

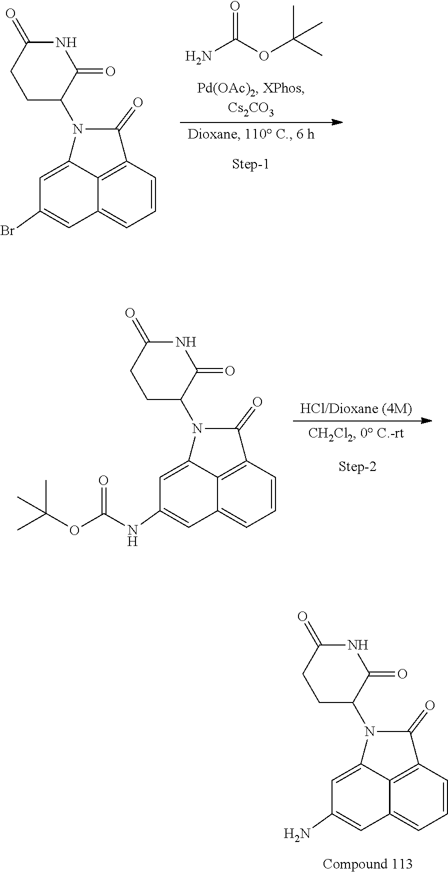

Step 1: To a suspension of 3-(7-bromo-2-oxo-benzo[cd]indol-1-yl)piperidine-2,6-dione (150 mg, 417.63 μmol) and tert-butyl carbamate (195.69 mg, 1.67 mmol) in 1,4-dioxane (10 mL), were added cesium carbonate (408.21 mg, 1.25 mmol), 2-dicyclohexylphosphino-2′,4′,6′-triisopropylbiphenyl (49.77 mg, 104.41 μmol) and palladium (II) acetate (23.44 mg, 104.41 μmol) and purged with nitrogen for 5 min. The contents were heated at 110° C. for 16 h. The reaction mixture was cooled to ambient temperature and filtered through a pad of celite. The filtrate was concentrated under reduced pressure to give tert-butyl N-[1-(2,6-dioxo-3-piperidyl)-2-oxo-benzo[cd]indol-7-yl]carbamate (130 mg, 289.32 μmol, 69.28% yield) as a pale yellow solid. LCMS (ES⁺) m/z 394.0 [M−H]⁺, RT=0.96 min. The crude product was taken to the next step without purification.

Step 2: To a solution of tert-butyl N-[1-(2,6-dioxo-3-piperidyl)-2-oxo-benzo[cd]indol-7-yl]carbamate (60 mg, 151.74 μmol) in dichloromethane (3 mL), cooled to 0° C., was added HCl in 1,4-dioxane (4 M, 1 mL). The resulting mixture was stirred at room temperature for 2 h. The reaction mixture was concentrated under reduced pressure to give the crude product. The crude product was purified by prep HPLC [Column: X bridge C18 (150×10) mm, 5 microns, Mobile phase: A: 0.1% HCOOH in water, B: Acetonitrile] and the fractions containing the product was lyophilized to give 3-(7-amino-2-oxo-benzo[cd]indol-1-yl)piperidine-2,6-dione Compound 113 (10 mg, 32.90 μmol, 21.68% yield) as an off-white solid. LCMS (ES⁺): m/z 296.2 [M+H]⁺.

| | | Compound | |
|---|---|---|---|
| Starting Material | Product | No. | LCMS |
| 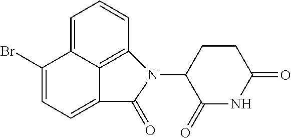 [SMILES: O=C1CCC(N2C(=O)c3ccc(Br)c4cccc2c34)C(=O)N1] | 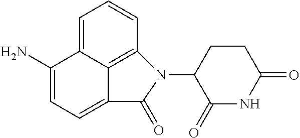 [SMILES: Nc1ccc2c3c(cccc13)N(C1CCC(=O)NC1=O)C2=O] | Compound 114 | 296.2 [M + H]⁺ |
| 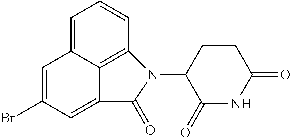 [SMILES: O=C1CCC(N2C(=O)c3cc(Br)cc4cccc2c34)C(=O)N1] | 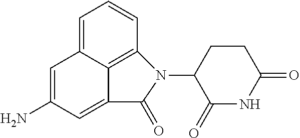 [SMILES: Nc1cc2c3c(cccc3c1)N(C1CCC(=O)NC1=O)C2=O] | Compound 115 | 296.1 [M + H]⁺ |

### Example 83. 3-(7-methyl-2-oxo-benzo[cd]indol-1-yl)piperidine-2,6-dione (Compound 116)

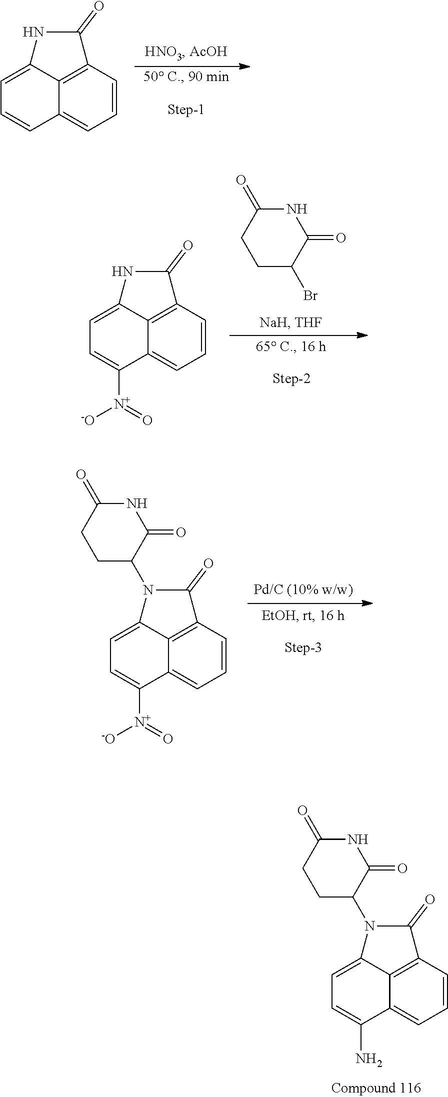

Step 1: To a solution of 1H-benzo[cd]indol-2-one (4 g, 23.64 mmol) in acetic acid (17 mL), was added nitric acid (1.92 g, 30.50 mmol) drop-wise. The resulting mixture was heated at 50° C. for 90 min. The reaction mixture was brought to room temperature, the precipitated solid was filtered, washed with aqueous acetic acid and dried under vacuum to give 6-nitro-1H-benzo[cd]indol-2-one (3.7 g, 13.87 mmol, 58.66% yield) as a pale-yellow solid. LCMS (ES⁺): m/z 213.0 [M−H]⁺. The crude product was taken to the next step without purification.

Step 2: To a solution of 6-nitro-1H-benzo[cd]indol-2-one (500 mg, 2.33 mmol) in tetrahydrofuran (20 mL), cooled to 0° C., was added sodium hydride (60% in mineral oil, 425.05 mg, 17.71 mmol). The resulting suspension was stirred at room temperature for 1 h. The mixture was cooled to 0° C., was added 3-bromopiperidine-2,6-dione (1.43 g, 7.47 mmol) in tetrahydrofuran (5 mL). The contents were heated at 65° C. for 16 h. The crude mixture was cooled to 0° C., treated with saturated ammonium chloride solution (10 mL) and extracted with ethyl acetate (3×30 mL). The combined organics were washed with brine solution (10 mL) and dried over anhydrous sodium sulfate. The solution was filtered and concentrated under reduced pressure to give the crude product. The crude product was purified by flash chromatography (silica gel, 230-400 mesh) eluted with 60-70% ethyl acetate in petroleum ether to give 3-(6-nitro-2-oxo-benzo[cd]indol-1-yl)piperidine-2,6-dione (200 mg, 527.68 μmol, 22.60% yield) as a brown solid. LCMS (ES⁺): m/z 324.0 [M−H]⁺, RT=0.82 min.

Step 3: To a solution of 3-(6-nitro-2-oxo-benzo[cd]indol-1-yl)piperidine-2,6-dione (50 mg, 153.72 μmol) in ethanol (3 mL) and tetrahydrofuran (2 mL), was added palladium, 10% on activated carbon powder (32.72 mg, 307.43 μmol). The contents were stirred under hydrogen atmosphere at room temperature for 16 h. The reaction mixture was filtered through a pad of celite, and the filtrate was concentrated under reduced pressure. The residue was triturated with methyl tert-butyl ether to give the crude product which was purified by preparative HPLC [Column: X select C18 (250×19) mm, 5 microns, Mobile phase: A: 0.1% HCOOH in water, B: Acetonitrile] and the fractions containing the product was lyophilized to give 3-(6-amino-2-oxo-benzo[cd]indol-1-yl)piperidine-2,6-dione Compound 116 (15 mg, 50.07 μmol, 32.57% yield) as an orange solid. LCMS (ES⁺): m/z 296.0 [M+H]⁺, RT=1.37 min.

### Example 84. 3-(6-chloro-2-oxo-benzo[cd]indol-1-yl)piperidine-2,6-dione (Compound 117)

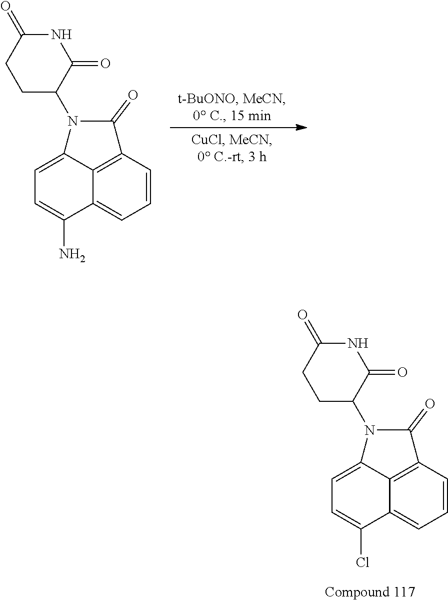 [SMILES: Nc1ccc2c3c(cccc13)C(=O)N2C1CCC(=O)NC1=O ; O=C1CCC(N2C(=O)c3cccc4c(Cl)ccc2c34)C(=O)N1]

To a solution of 3-(6-amino-2-oxo-benzo[cd]indol-1-yl)piperidine-2,6-dione (210 mg, 711.16 μmol) in acetonitrile (6 mL), cooled to 0° C., was added tert-butyl nitrite (110.00 mg, 1.07 mmol, 126.88 μL). The resulting mixture was stirred at 0° C. for 15 min. To this mixture, was added copper(I) chloride (105.61 mg, 1.07 mmol) and the contents were stirred at room temperature for 3 h. The crude mixture was cooled to 0° C., partitioned between ethyl acetate (10 mL)/water (5 ml) and extracted using ethyl acetate (2×5 mL). The combined organics were washed with brine solution (10 mL) dried over anhydrous sodium sulfate. The solution was filtered and concentrated under reduced pressure to give the crude product. The crude product was purified by flash chromatography (silica gel, 230-400 mesh) eluted with 60-75% ethyl acetate in petroleum ether and the product was purified again using reverse phase preparative HPLC [Column: Sunfire C18 (19×150) mm, 5 microns; Mobile phase: A: 0.1% HCOOH in water, B: Acetonitrile]. The fractions containing the product was combined and lyophilized to give 3-(6-chloro-2-oxo-benzo[cd]indol-1-yl)piperidine-2,6-dione Compound 117 (7 mg, 21.08 μmol, 2.96% yield) as a pale yellow solid. LCMS (ES⁺): m/z 315.0 [M+H]⁺, 0.90 min.

| | | Compound | |
|---|---|---|---|
| Starting Material | Product | No. | LCMS |
| 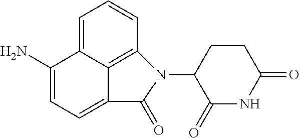 [SMILES: Nc1ccc2c3c(cccc13)N(C1CCC(=O)NC1=O)C2=O] | 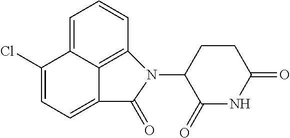 [SMILES: O=C1CCC(N2C(=O)c3ccc(Cl)c4cccc2c34)C(=O)N1] | Compound 118 | 313.0 [M − H]⁻ |
| 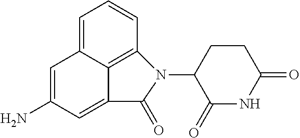 [SMILES: Nc1cc2c3c(cccc3c1)N(C1CCC(=O)NC1=O)C2=O] | 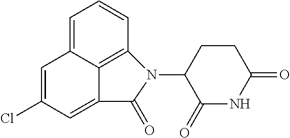 [SMILES: O=C1CCC(N2C(=O)c3cc(Cl)cc4cccc2c34)C(=O)N1] | Compound 119 | 312.9 [M − H]⁻ |

### Example 85. 3-(7-chloro-2-oxo-benzo[cd]indol-1-yl)piperidine-2,6-dione (Compound 120)

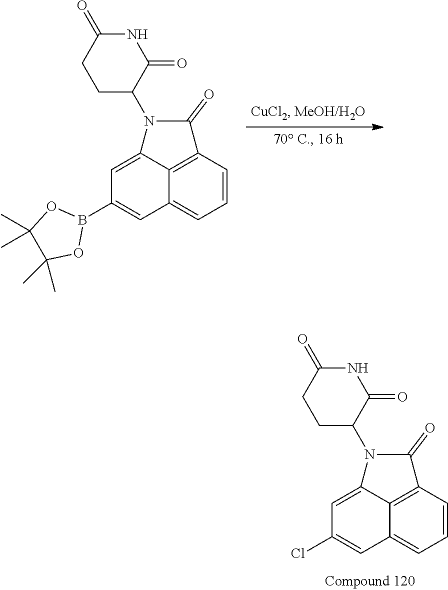 [SMILES: CC1(C)OB(c2cc3c4c(cccc4c2)C(=O)N3C2CCC(=O)NC2=O)OC1(C)C ; O=C1CCC(N2C(=O)c3cccc4cc(Cl)cc2c34)C(=O)N1]

To a solution of 3-[2-oxo-7-(4,4,5,5-tetramethyl-1,3,2-dioxaborolan-2-yl)benzo[cd]indol-1-yl]piperidine-2,6-dione (200 mg, 207.61 μmol) in methanol (5 mL) and water (0.5 mL), was added copper(II) chloride (139.57 mg, 1.04 mmol, 41.17 μL). The contents were heated at 70° C. for 16 h. The reaction mixture was treated with water (10 mL) and extracted with ethyl acetate (2×30 mL). The combined organics were washed with brine solution (20 mL) and dried over anhydrous sodium sulfate. The solution was filtered and concentrated under reduced pressure to give the crude product. The crude product was purified by preparative HPLC [Column: X Select C18 (250×19) mm, 5 microns, Mobile phase: A 0.1% Formic acid, B: Acetonitrile] and the fractions containing the product was combined and lyophilized to give 3-(7-chloro-2-oxo-benzo[cd]indol-1-yl)piperidine-2,6-dione Compound 120 (23 mg, 70.73 μmol, 34.07% yield) as an off-white solid. LCMS (ES⁺): m/z 315.0 [M+H]⁺, RT=0.89 min.

### Example 86. 3-(7-fluoro-2-oxo-benzo[cd]indol-1-yl)piperidine-2,6-dione (Compound 121)

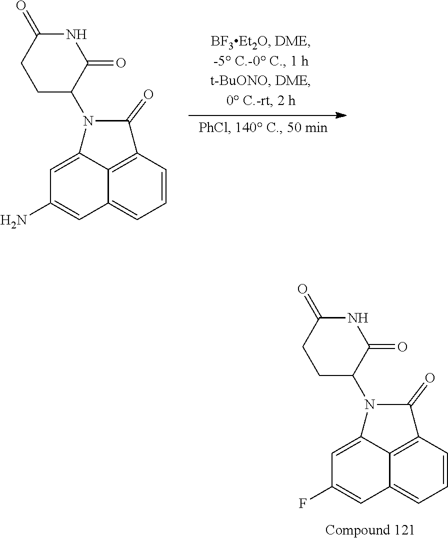 [SMILES: Nc1cc2c3c(cccc3c1)C(=O)N2C1CCC(=O)NC1=O ; O=C1CCC(N2C(=O)c3cccc4cc(F)cc2c34)C(=O)N1]

To a solution of boron trifluoride diethyl etherate (205.35 mg, 723.43 μmol, 186.68 μL) in 1,2-dimethoxyethane (10 mL), cooled to −5° C., was added 3-(7-amino-2-oxo-benzo[cd]indol-1-yl)piperidine-2,6-dione hydrochloride (200.0 mg, 602.86 μmol) in 1,2-dimethoxyethane (6 mL) drop-wise over 30 min. The resulting mixture was stirred at 0° C. for 1 h. The mixture was cooled to 0° C., was added tert-butyl nitrite (62.17 mg, 602.86 μmol, 71.70 μL) in 1,2-dimethoxyethane (4 mL). The resulting mixture was stirred at room temperature for 2 h. The reaction mixture was concentrated under reduced pressure, the residue was dissolved in chlorobenzene (5 mL) and the resulting mixture was heated at 140° C. for 50 min. The reaction mixture was cooled to room temperature and concentrated under reduced pressure. The residue was treated with dichloromethane (10 mL) and washed with 10% sodium bicarbonate solution (5 mL). The organic phase was dried over anhydrous sodium sulfate, the solution was filtered and concentrated under reduced pressure to give the crude product. The crude product was purified by preparative HPLC [Column: Sunfire C18 (19×150) mm, 5 microns; Mobile phase: A: 0.1% HCOOH in water, B: Acetonitrile] and the fractions containing the product was combined and lyophilized to give 3-(7-fluoro-2-oxo-benzo[cd]indol-1-yl)piperidine-2,6-dione Compound 121 (2.35 mg, 7.25 μmol, 1.20% yield) as a pale yellow solid. LCMS (ES⁺): m/z 297.0 [M−H], RT=2.21 min. ¹H NMR (400 MHz, DMSO-d₆): δ 11.14 (s, 1H), 8.22 (d, J=8.0 Hz, 1H), 8.09 (d, J=7.2 Hz, 1H), 7.88 (t, J=7.6 Hz, 1H), 7.47 (dd, J=11.0, 2.0 Hz, 1H), 7.27 (dd, J=9.6, 1.6 Hz, 1H), 5.50-5.45 (m, 1H), 2.89-2.76 (m, 2H), 2.68-2.64 (m, 1H), 2.14-2.08 (m, 1H) ppm.

| | | Compound | |
|---|---|---|---|
| Starting Material | Product | No. | LCMS |
| 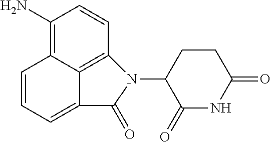 [SMILES: Nc1ccc2c3c(cccc13)C(=O)N2C1CCC(=O)NC1=O] | 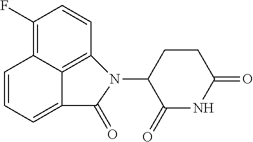 [SMILES: O=C1CCC(N2C(=O)c3cccc4c(F)ccc2c34)C(=O)N1] | Compound 122 | 299.0 [M + H]⁺ |
| 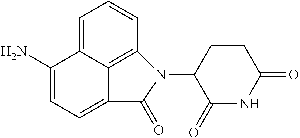 [SMILES: Nc1ccc2c3c(cccc13)N(C1CCC(=O)NC1=O)C2=O] | 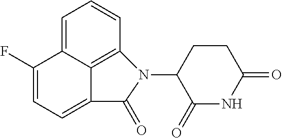 [SMILES: O=C1CCC(N2C(=O)c3ccc(F)c4cccc2c34)C(=O)N1] | Compound 123 | 297.1 [M − H]⁻ |
| 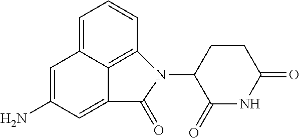 [SMILES: Nc1cc2c3c(cccc3c1)N(C1CCC(=O)NC1=O)C2=O] | 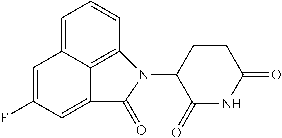 [SMILES: O=C1CCC(N2C(=O)c3cc(F)cc4cccc2c34)C(=O)N1] | Compound 124 | 299.0 [M + H]⁺ |

### Example 87. 3-(7-hydroxy-2-oxo-benzo[cd]indol-1-yl)piperidine-2,6-dione (Compound 125)

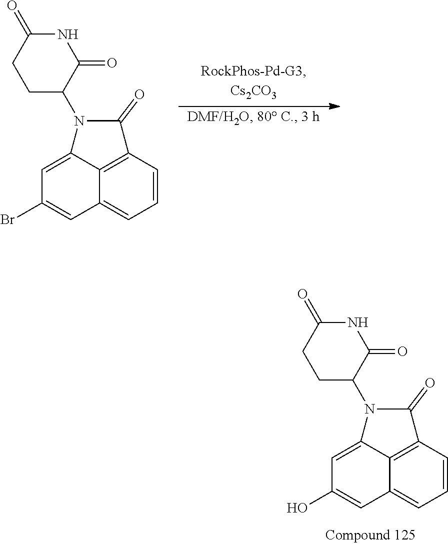 [SMILES: O=C1CCC(N2C(=O)c3cccc4cc(Br)cc2c34)C(=O)N1 ; O=C1CCC(N2C(=O)c3cccc4cc(O)cc2c34)C(=O)N1]

Step 1: To a solution of 3-(7-bromo-2-oxo-benzo[cd]indol-1-yl)piperidine-2,6-dione (70.00 mg, 194.89 μmol) in N,N-dimethylformamide (2 mL), were added water (14.05 mg, 779.57 μmol, 14.05 μL) and cesium carbonate (190.50 mg, 584.68 μmol). The contents were degassed with nitrogen for 5 min. To this mixture, was added RockPhos-Pd-G3 (32.68 mg, 38.98 μmol). The contents were heated to 80° C. for 3 h. The reaction mixture was directly loaded in reverse phase C₁₈ column [ISCO column (30 g), Mobile phase: A: 0.1% HCOOH in water, B: Acetonitrile] and the fractions containing the product was lyophilized to give 3-(7-hydroxy-2-oxo-benzo[cd]indol-1-yl)piperidine-2,6-dione Compound 125 (12 mg, 39.14 μmol, 20.08% yield) as an off-white solid. LCMS (ES⁺): m/z 295.0 [M−H]⁺.

| Starting Material | Product | Compound No. | LCMS |
|---|---|---|---|
| 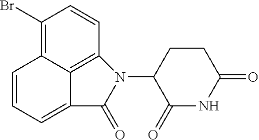 [SMILES: O=C1CCC(N2C(=O)c3cccc4c(Br)ccc2c34)C(=O)N1] | 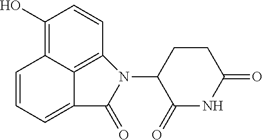 [SMILES: O=C1CCC(N2C(=O)c3cccc4c(O)ccc2c34)C(=O)N1] | Compound 126 | 297.2 [M + H] ⁺ |
| 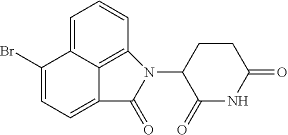 [SMILES: O=C1CCC(N2C(=O)c3ccc(Br)c4cccc2c34)C(=O)N1] | 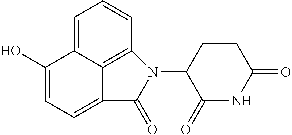 [SMILES: O=C1CCC(N2C(=O)c3ccc(O)c4cccc2c34)C(=O)N1] | Compound 127 | 297.2 [M + H] ⁺ |
| 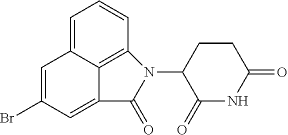 [SMILES: O=C1CCC(N2C(=O)c3cc(Br)cc4cccc2c34)C(=O)N1] | 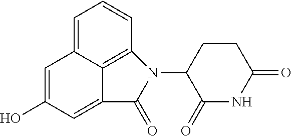 [SMILES: O=C1CCC(N2C(=O)c3cc(O)cc4cccc2c34)C(=O)N1] | Compound 128 | 297.0 [M + H] ⁺ |

### Example 88. 3-(6-bromo-2-oxo-benzo[cd]indol-1-yl)piperidine-2,6-dione (Compound 129)

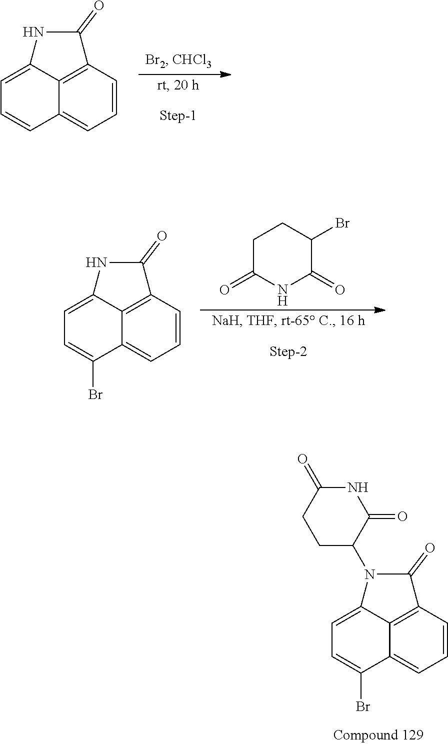 [SMILES: O=C1Nc2cccc3cccc1c23 ; O=C1Nc2ccc(Br)c3cccc1c23 ; O=C1CCC(Br)C(=O)N1 ; O=C1CCC(N2C(=O)c3cccc4c(Br)ccc2c34)C(=O)N1]

Step 1: To a solution of 1H-benzo[cd]indol-2-one (5.00 g, 29.55 mmol) in chloroform (300 mL), cooled to 0° C., was added bromine (3.59 g, 44.38 mmol, 2.41 mL). The reaction mixture was stirred at room temperature for 20 h. The reaction mixture was treated with ice-cold sodium thiosulfate solution (200 mL). The precipitated solid was filtered, washed with cold water (250 mL), diethyl ether (150 mL) and dried under vacuum to give 6-bromo-1H-benzo[cd]indol-2-one (5.4 g, 20.59 mmol, 69.66% yield) as a yellow solid. LCMS (ES⁺): m/z 247.9 [M+H]⁺.

Step 2: To a solution of 6-bromo-1H-benzo[cd]indol-2-one (2.00 g, 8.06 mmol) in tetrahydrofuran (150 mL), cooled to 0° C., was added sodium hydride (60% dispersion in mineral oil, 1.60 g, 41.76 mmol). The contents were stirred at room temperature for 1 h. The mixture was cooled to 0° C., and 3-bromopiperidine-2,6-dione (3.87 g, 20.16 mmol) in tetrahydrofuran (10 mL) was added dropwise. The resulting mixture was heated at 60° C. for 16 h. The reaction mixture was cooled to 0° C., treated with saturated ammonium chloride solution slowly and extracted with ethyl acetate (2×100 mL). The combined organics were washed with brine solution (80 mL) and dried over anhydrous sodium sulfate. The solution was filtered and concentrated under reduced pressure to give the crude product. The crude product was recrystallized with dichloromethane (10 mL), filtered, washed with dichloromethane and dried to give 3-(6-bromo-2-oxo-benzo[cd]indol-1-yl)piperidine-2,6-dione (1.9 g, 4.94 mmol, 61.22% yield) as a yellow solid. LCMS (ES⁺): m/z 359.0 [M+H]⁺.

### Example 89. Synthesis of 3-(6-hydroxy-2-oxopyrrolo[4,3,2-de]quinolin-1(2H)-yl)piperidine-2,6-dione (Compound 130)

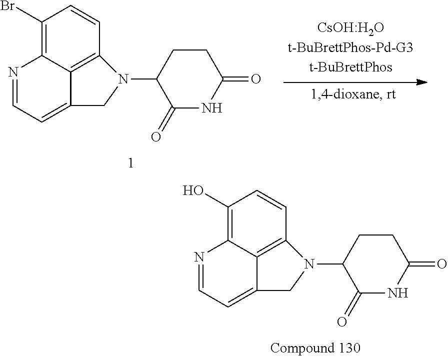 [SMILES: O=C1CCC(N2Cc3ccnc4c(Br)ccc2c34)C(=O)N1 ; O=C1CCC(N2Cc3ccnc4c(O)ccc2c34)C(=O)N1]

Procedures are utilized as reported by Cheung et. al. J. Org. Chem. 2014, 79, 5351-5358.

To a stirred solution of t-BuBrettPhos (0.02 eq) and aryl bromide 1 (1 eq) is added CsOH aqueous solution (CsOH:H₂O mole ratio 3:10, 3 eq) under an inert atmosphere. A second solution of t-BuBrettPhos-Pd-G3 (0.02 eq) in 1,4-dioxane (0.5 M) is then added to the reaction vessel. The mixture is stirred at room temperature until reaction completion is evident. Upon reaction completion, the mixture is diluted with EtOAc and acidified with aqueous HCl solution (1 M). The layers are then neutralized with saturated NaHCO₃ solution before the aqueous layer is further extracted three times with EtOAc. The combined organic layers are dried over anhydrous sodium sulfate and filtered. The filtrate is then concentrated under reduced pressure to a residue. The crude residue is purified using silica gel column chromatography to afford the product 3-(6-hydroxy-2-oxopyrrolo[4,3,2-de]quinolin-1(2H)-yl)piperidine-2,6-dione Compound 130.

Using similar conditions, the non-limiting starting materials below on the left can be converted to the corresponding products on the right.

| Starting Material | Product | Compound No. |
|---|---|---|
| 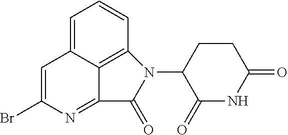 [SMILES: O=C1CCC(N2C(=O)c3nc(Br)cc4cccc2c34)C(=O)N1] | 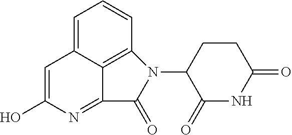 [SMILES: O=C1CCC(N2C(=O)c3nc(O)cc4cccc2c34)C(=O)N1] | Compound 131 |
| 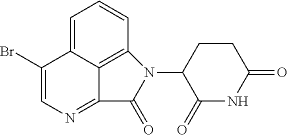 [SMILES: O=C1CCC(N2C(=O)c3ncc(Br)c4cccc2c34)C(=O)N1] | 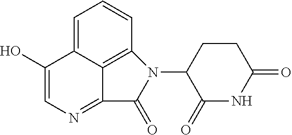 [SMILES: O=C1CCC(N2C(=O)c3ncc(O)c4cccc2c34)C(=O)N1] | Compound 132 |
| 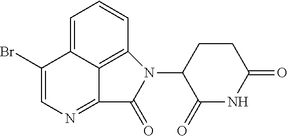 [SMILES: O=C1CCC(N2C(=O)c3ncc(Br)c4cccc2c34)C(=O)N1] | 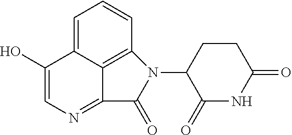 [SMILES: O=C1CCC(N2C(=O)c3ncc(O)c4cccc2c34)C(=O)N1] | Compound 133 |
| 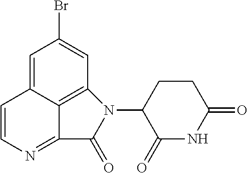 [SMILES: O=C1CCC(N2C(=O)c3nccc4cc(Br)cc2c34)C(=O)N1] | 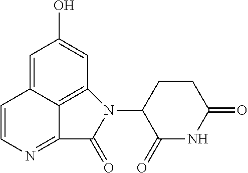 [SMILES: O=C1CCC(N2C(=O)c3nccc4cc(O)cc2c34)C(=O)N1] | Compound 134 |
| 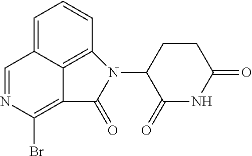 [SMILES: O=C1CCC(N2C(=O)c3c(Br)ncc4cccc2c34)C(=O)N1] | 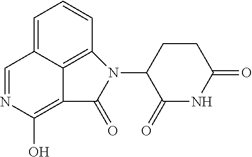 [SMILES: O=C1CCC(N2C(=O)c3c(O)ncc4cccc2c34)C(=O)N1] | Compound 135 |
| 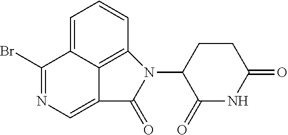 [SMILES: O=C1CCC(N2C(=O)c3cnc(Br)c4cccc2c34)C(=O)N1] | 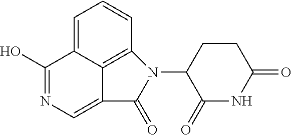 [SMILES: O=C1CCC(N2C(=O)c3cnc(O)c4cccc2c34)C(=O)N1] | Compound 136 |
| 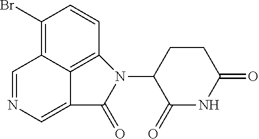 [SMILES: O=C1CCC(N2C(=O)c3cncc4c(Br)ccc2c34)C(=O)N1] | 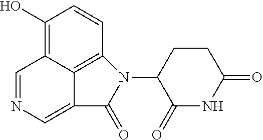 [SMILES: O=C1CCC(N2C(=O)c3cncc4c(O)ccc2c34)C(=O)N1] | Compound 137 |
| 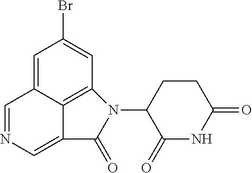 [SMILES: O=C1CCC(N2C(=O)c3cncc4cc(Br)cc2c34)C(=O)N1] | 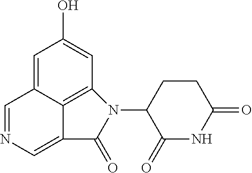 [SMILES: O=C1CCC(N2C(=O)c3cncc4cc(O)cc2c34)C(=O)N1] | Compound 138 |
| 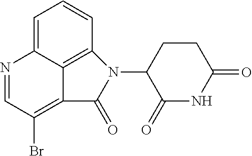 [SMILES: O=C1CCC(N2C(=O)c3c(Br)cnc4cccc2c34)C(=O)N1] | 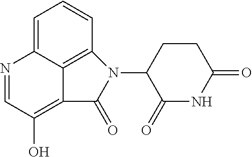 [SMILES: O=C1CCC(N2C(=O)c3c(O)cnc4cccc2c34)C(=O)N1] | Compound 139 |
| 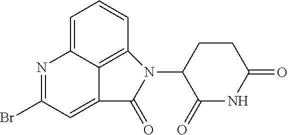 [SMILES: O=C1CCC(N2C(=O)c3cc(Br)nc4cccc2c34)C(=O)N1] | 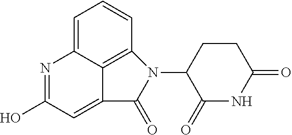 [SMILES: O=C1CCC(N2C(=O)c3cc(O)nc4cccc2c34)C(=O)N1] | Compound 140 |
| 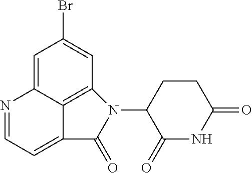 [SMILES: O=C1CCC(N2C(=O)c3ccnc4cc(Br)cc2c34)C(=O)N1] | 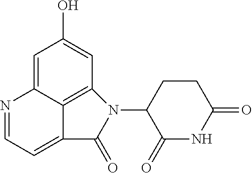 [SMILES: O=C1CCC(N2C(=O)c3ccnc4cc(O)cc2c34)C(=O)N1] | Compound 141 |
| 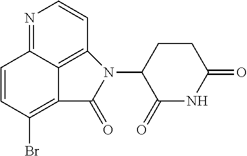 [SMILES: O=C1CCC(N2C(=O)c3c(Br)ccc4nccc2c34)C(=O)N1] | 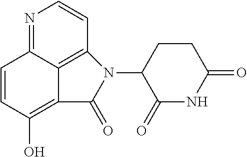 [SMILES: O=C1CCC(N2C(=O)c3c(O)ccc4nccc2c34)C(=O)N1] | Compound 142 |
| 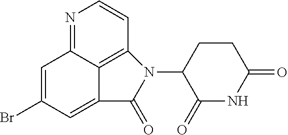 [SMILES: O=C1CCC(N2C(=O)c3cc(Br)cc4nccc2c34)C(=O)N1] | 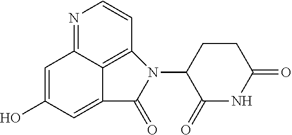 [SMILES: O=C1CCC(N2C(=O)c3cc(O)cc4nccc2c34)C(=O)N1] | Compound 143 |
| 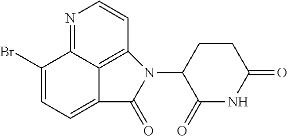 [SMILES: O=C1CCC(N2C(=O)c3ccc(Br)c4nccc2c34)C(=O)N1] | 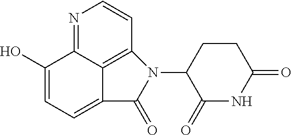 [SMILES: O=C1CCC(N2C(=O)c3ccc(O)c4nccc2c34)C(=O)N1] | Compound 144 |
| 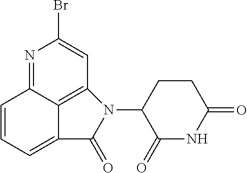 [SMILES: O=C1CCC(N2C(=O)c3cccc4nc(Br)cc2c34)C(=O)N1] | 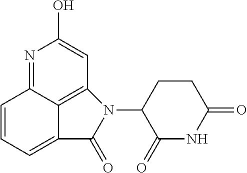 [SMILES: O=C1CCC(N2C(=O)c3cccc4nc(O)cc2c34)C(=O)N1] | Compound 145 |
| 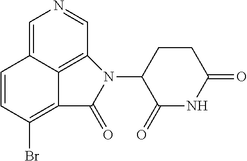 [SMILES: O=C1CCC(N2C(=O)c3c(Br)ccc4cncc2c34)C(=O)N1] | 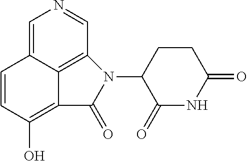 [SMILES: O=C1CCC(N2C(=O)c3c(O)ccc4cncc2c34)C(=O)N1] | Compound 146 |
| 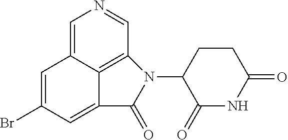 [SMILES: O=C1CCC(N2C(=O)c3cc(Br)cc4cncc2c34)C(=O)N1] | 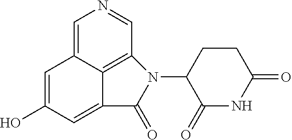 [SMILES: O=C1CCC(N2C(=O)c3cc(O)cc4cncc2c34)C(=O)N1] | Compound 147 |
| 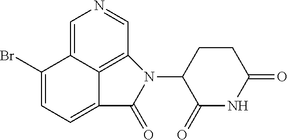 [SMILES: O=C1CCC(N2C(=O)c3ccc(Br)c4cncc2c34)C(=O)N1] | 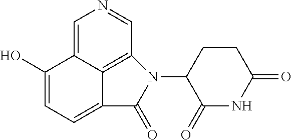 [SMILES: O=C1CCC(N2C(=O)c3ccc(O)c4cncc2c34)C(=O)N1] | Compound 148 |
| 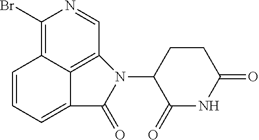 [SMILES: O=C1CCC(N2C(=O)c3cccc4c(Br)ncc2c34)C(=O)N1] | 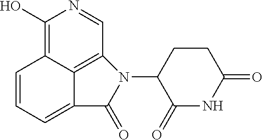 [SMILES: O=C1CCC(N2C(=O)c3cccc4c(O)ncc2c34)C(=O)N1] | Compound 149 |
| 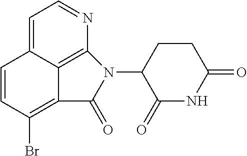 [SMILES: O=C1CCC(N2C(=O)c3c(Br)ccc4ccnc2c34)C(=O)N1] | 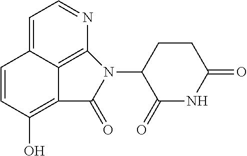 [SMILES: O=C1CCC(N2C(=O)c3c(O)ccc4ccnc2c34)C(=O)N1] | Compound 150 |
| 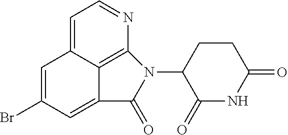 [SMILES: O=C1CCC(N2C(=O)c3cc(Br)cc4ccnc2c34)C(=O)N1] | 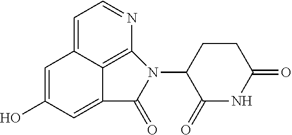 [SMILES: O=C1CCC(N2C(=O)c3cc(O)cc4ccnc2c34)C(=O)N1] | Compound 151 |
| 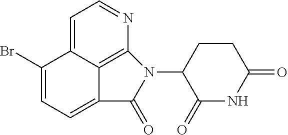 [SMILES: O=C1CCC(N2C(=O)c3ccc(Br)c4ccnc2c34)C(=O)N1] | 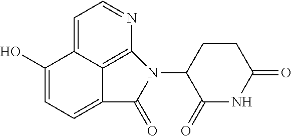 [SMILES: O=C1CCC(N2C(=O)c3ccc(O)c4ccnc2c34)C(=O)N1] | Compound 152 |
| 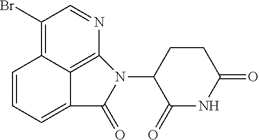 [SMILES: O=C1CCC(N2C(=O)c3cccc4c(Br)cnc2c34)C(=O)N1] | 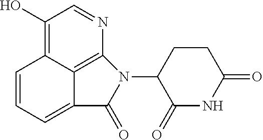 [SMILES: O=C1CCC(N2C(=O)c3cccc4c(O)cnc2c34)C(=O)N1] | Compound 153 |
| 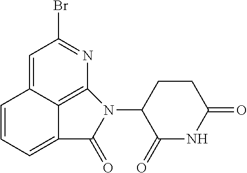 [SMILES: O=C1CCC(N2C(=O)c3cccc4cc(Br)nc2c34)C(=O)N1] | 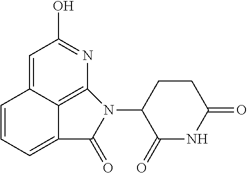 [SMILES: O=C1CCC(N2C(=O)c3cccc4cc(O)nc2c34)C(=O)N1] | Compound 154 |

### Example 90. 3-[5-[[(2,5-difluorophenyl)methylamino]methyl]-2-oxo-benzo[cd]indol-1-yl]piperidine-2,6-dione (Compound 155)

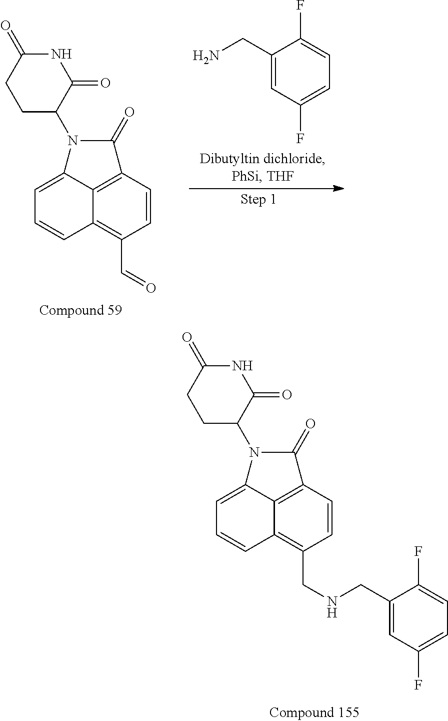 [SMILES: O=Cc1ccc2c3c(cccc13)N(C1CCC(=O)NC1=O)C2=O ; NCc1cc(F)ccc1F ; O=C1CCC(N2C(=O)c3ccc(CNCc4cc(F)ccc4F)c4cccc2c34)C(=O)N1]

Step 1: To the stirred solution of 1-(2,6-dioxo-3-piperidyl)-2-oxo-benzo[cd]indole-5-carbaldehyde Compound 59 (100 mg, 324 μmol, 1 eq) in THE (2 mL) was added (2,5-difluorophenyl)methanamine (46 mg, 324 μmol, 1 eq) followed by the addition of dibutyltin dichloride (118 mg, 389 μmol, 1.2 eq) and phenylsilane (35 mg, 324 μmol, 1 eq) and the reaction mixture was heated at 70° C. for 16 hours. After completion of the reaction (monitored by LCMS) the reaction mixture was evaporated and submitted for prep HPLC (reverse phase) to afford3-[5-[[(2,5-difluorophenyl)methylamino]methyl]-2-oxo-benzo[cd]indol-1-yl]piperidine-2,6-dione Compound 155 (18 mg, 0.041 mmol, 12.6% yield) was isolated. LCMS (ESI): m/z 436.1 [M+H]⁺.

Using similar conditions, the non-limiting starting materials below on the left can be converted to the corresponding products on the right.

| Starting | | Compound | |
|---|---|---|---|
| Material | Product | No. | LCMS |
| Compound 59 | 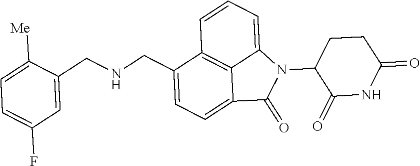 [SMILES: Cc1ccc(F)cc1CNCc1ccc2c3c(cccc13)N(C1CCC(=O)NC1=O)C2=O] | Compound 156 | 432.4 |
| Compound 59 | 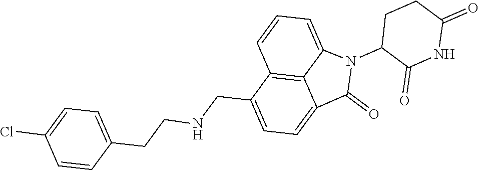 [SMILES: O=C1CCC(N2C(=O)c3ccc(CNCCc4ccc(Cl)cc4)c4cccc2c34)C(=O)N1] | Compound 157 | 488.1 |
| Compound 59 | 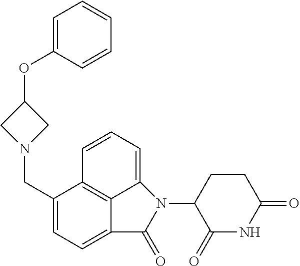 [SMILES: O=C1CCC(N2C(=O)c3ccc(CN4CC(Oc5ccccc5)C4)c4cccc2c34)C(=O)N1] | Compound 63 | 442.4 |
| Compound 55 | 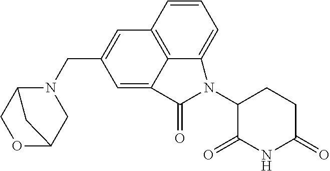 [SMILES: O=C1CCC(N2C(=O)c3cc(CN4CC5CC4CO5)cc4cccc2c34)C(=O)N1] | Compound 159 | 392.4 |
| Compound 55 | 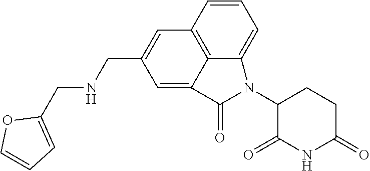 [SMILES: O=C1CCC(N2C(=O)c3cc(CNCc4ccco4)cc4cccc2c34)C(=O)N1] | Compound 160 | 390.1 |
| Compound 55 | 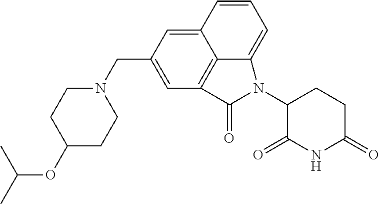 [SMILES: CC(C)OC1CCN(Cc2cc3c4c(cccc4c2)N(C2CCC(=O)NC2=O)C3=O)CC1] | Compound 161 | 436.2 |
| Compound 55 | 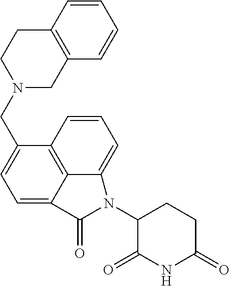 [SMILES: O=C1CCC(N2C(=O)c3ccc(CN4CCc5ccccc5C4)c4cccc2c34)C(=O)N1] | Compound 162 | 426.4 |
| Compound 55 | 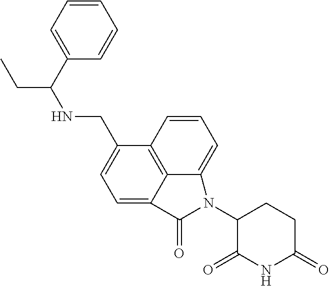 [SMILES: CCC(NCc1ccc2c3c(cccc13)N(C1CCC(=O)NC1=O)C2=O)c1ccccc1] | Compound 163 | 428.1 |
| Compound 55 | 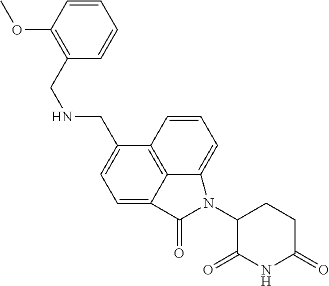 [SMILES: COc1ccccc1CNCc1ccc2c3c(cccc13)N(C1CCC(=O)NC1=O)C2=O] | Compound 164 | 429.9 |
| Compound 55 | 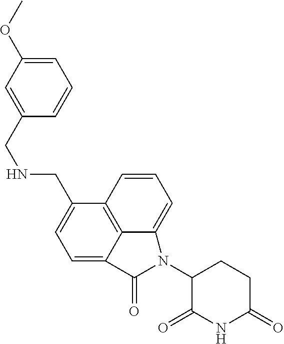 [SMILES: COc1cccc(CNCc2ccc3c4c(cccc24)N(C2CCC(=O)NC2=O)C3=O)c1] | Compound 165 | 430.1 |
| Compound 55 | 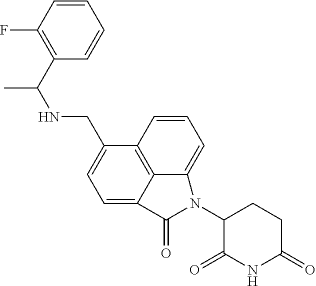 [SMILES: CC(NCc1ccc2c3c(cccc13)N(C1CCC(=O)NC1=O)C2=O)c1ccccc1F] | Compound 166 | 432.1 |
| Compound 55 | 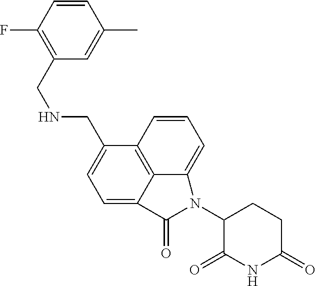 [SMILES: Cc1ccc(F)c(CNCc2ccc3c4c(cccc24)N(C2CCC(=O)NC2=O)C3=O)c1] | Compound 167 | 432.4 |
| Compound 55 | 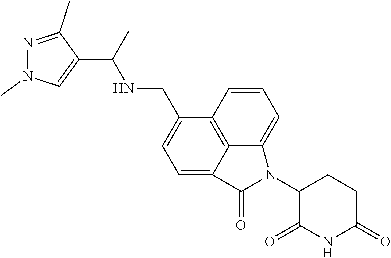 [SMILES: Cc1nn(C)cc1C(C)NCc1ccc2c3c(cccc13)N(C1CCC(=O)NC1=O)C2=O] | Compound 168 | 432.4 |
| Compound 55 | 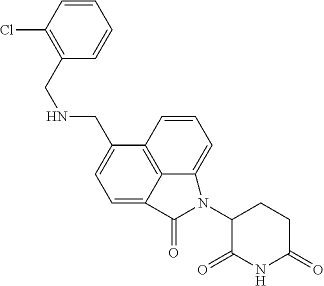 [SMILES: O=C1CCC(N2C(=O)c3ccc(CNCc4ccccc4Cl)c4cccc2c34)C(=O)N1] | Compound 169 | 434.3 |
| Compound 55 | 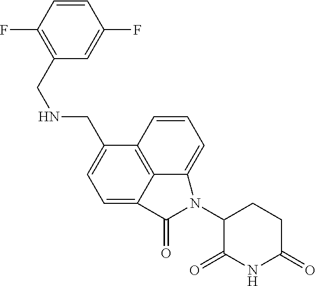 [SMILES: O=C1CCC(N2C(=O)c3ccc(CNCc4cc(F)ccc4F)c4cccc2c34)C(=O)N1] | Compound 170 | 436.1 |
| Compound 55 | 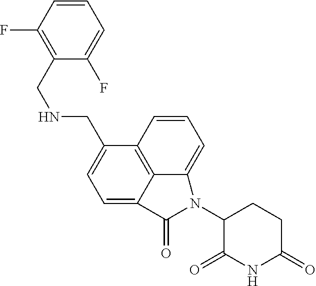 [SMILES: O=C1CCC(N2C(=O)c3ccc(CNCc4c(F)cccc4F)c4cccc2c34)C(=O)N1] | Compound 171 | 436.1 |
| Compound 55 | 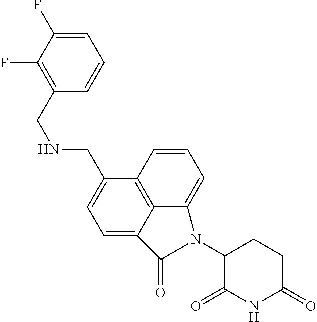 [SMILES: O=C1CCC(N2C(=O)c3ccc(CNCc4cccc(F)c4F)c4cccc2c34)C(=O)N1] | Compound 172 | 436.1 |
| Compound 55 | 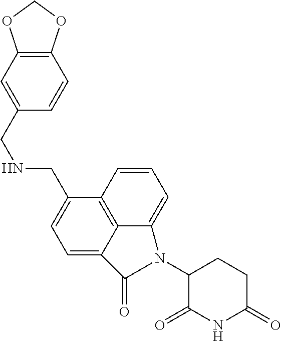 [SMILES: O=C1CCC(N2C(=O)c3ccc(CNCc4ccc5c(c4)OCO5)c4cccc2c34)C(=O)N1] | Compound 173 | 444.1 |
| Compound 55 | 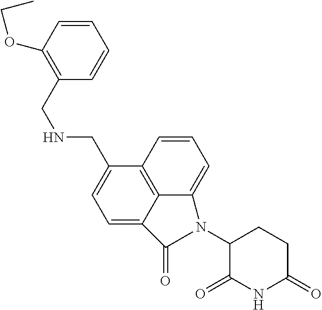 [SMILES: CCOc1ccccc1CNCc1ccc2c3c(cccc13)N(C1CCC(=O)NC1=O)C2=O] | Compound 174 | 444.2 |
| Compound 55 | 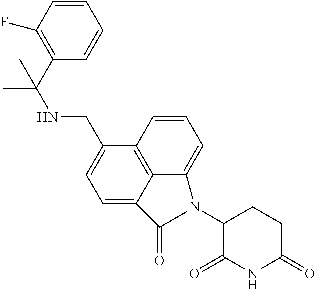 [SMILES: CC(C)(NCc1ccc2c3c(cccc13)N(C1CCC(=O)NC1=O)C2=O)c1ccccc1F] | Compound 175 | 446.5 |
| 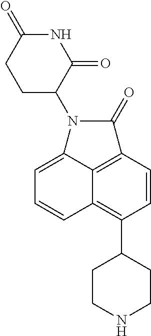 [SMILES: O=C1CCC(N2C(=O)c3ccc(C4CCNCC4)c4cccc2c34)C(=O)N1] | 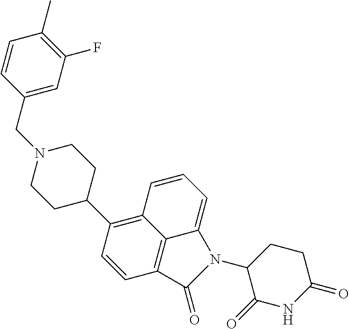 [SMILES: Cc1ccc(CN2CCC(c3ccc4c5c(cccc35)N(C3CCC(=O)NC3=O)C4=O)CC2)cc1F] | Compound 176 | 486.2 |
| 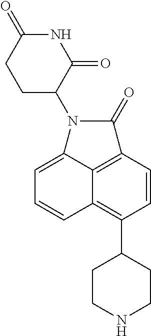 [SMILES: O=C1CCC(N2C(=O)c3ccc(C4CCNCC4)c4cccc2c34)C(=O)N1] | 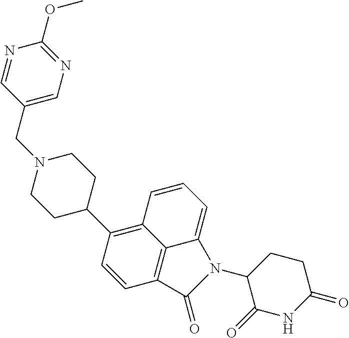 [SMILES: COc1ncc(CN2CCC(c3ccc4c5c(cccc35)N(C3CCC(=O)NC3=O)C4=O)CC2)cn1] | Compound 177 | 486.2 |
| 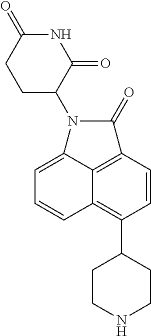 [SMILES: O=C1CCC(N2C(=O)c3ccc(C4CCNCC4)c4cccc2c34)C(=O)N1] | 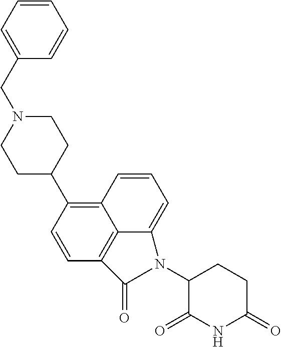 [SMILES: O=C1CCC(N2C(=O)c3ccc(C4CCN(Cc5ccccc5)CC4)c4cccc2c34)C(=O)N1] | Compound 178 | 454.2 |
| 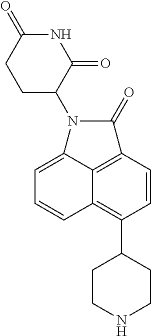 [SMILES: O=C1CCC(N2C(=O)c3ccc(C4CCNCC4)c4cccc2c34)C(=O)N1] | 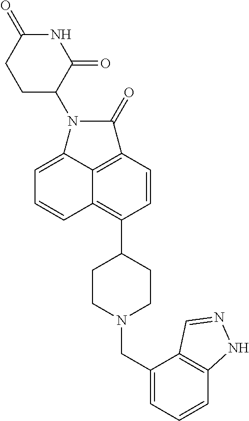 [SMILES: O=C1CCC(N2C(=O)c3ccc(C4CCN(Cc5cccc6[nH]ncc56)CC4)c4cccc2c34)C(=O)N1] | Compound 179 | 494.4 |

### Example 91. 1-(2,6-dioxo-3-piperidyl)-N-(2-methyl-1-phenyl-propyl)-2-oxo-benzo[cd]indole-5-carboxamide (Compound 180)

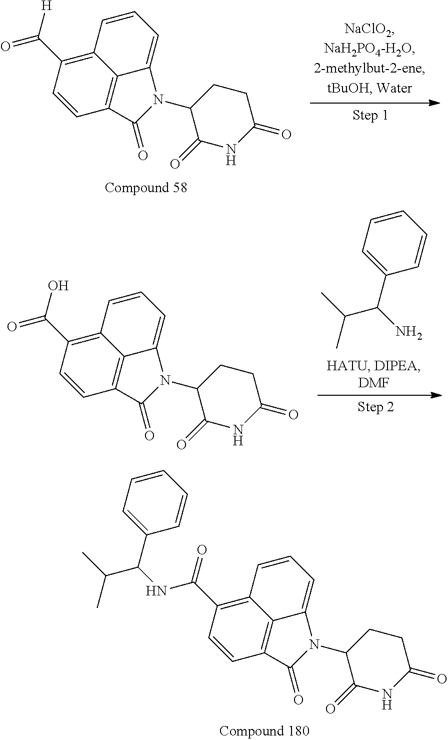

Step 1: To a stirred solution of compound 1-(2,6-dioxo-3-piperidyl)-2-oxo-benzo[cd]indole-5-carbaldehyde (200 mg, 648 μmol, 1 eq) in tert-butanol (12 mL) under ice water 2-methylbut-2-ene (682.45 mg, 9.73 mmol, 15 eq., 1.03 mL) was added. To this, an aqueous solution of sodium chlorite (293.36 mg, 3.24 mmol, 5 eq) and sodium dihydrogen phosphate monohydrate (447.61 mg, 324 mmol, 5 eq) was added dropwise and the stirring was continued for 16 h at RT. LCMS of the crude confirmed the product formation. The reaction mixture was evaporated to dryness and 10 mL of 10 (M) NaOH was added. Then the resulting solution was extracted with ethyl acetate and aqueous part was acidified using 1 (N) HCl solution. A yellow precipitate was appeared which was filtered off to get the 1-(2,6-dioxo-3-piperidyl)-2-oxo-benzo[cd]indole-5-carboxylic acid (120 mg, 336.74 μmol, 51.91% yield) LCMS (ESI): m/z 325.8 [M−H].

Step 2: To a stirred solution of 1-(2,6-dioxo-3-piperidyl)-2-oxo-benzo[cd]indole-5-carboxylic acid (38 mg, 117 μmol, 1 eq) in DMF (1.5 mL), HATU (44.56 mg, 117 μmol, 1 eq) was added and the reaction mixture was stirred for 5 min at 0° C. Then 2-methyl-1-phenyl-propan-1-amine (20.98 mg, 140 μmol 1.2 eq) and DIPEA (40.82 μL, 234 μmol, 2 eq) was added and the reaction was continued at RT for 16 h. Crude LCMS confirmed the product formation. The reaction mixture was directly submitted for reverse phase prep purification. The pure product 1-(2,6-dioxo-3-piperidyl)-N-(2-methyl-1-phenyl-propyl)-2-oxo-benzo[cd]indole-5-carboxamide Compound 180 (9.83 mg, 21.58 μmol, 18.42% yield) was isolated. LCMS (ESI): m/z 454.1 [M+H]⁺.

Using similar conditions, the products on the right were synthesized using the corresponding amines on the left.

| Amine | Product | Compound No. | LCMS |
|---|---|---|---|
| 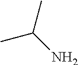 | 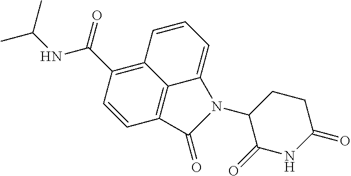 [SMILES: CC(C)NC(=O)c1ccc2c3c(cccc13)N(C1CCC(=O)NC1=O)C2=O] | Compound 181 | 366.0 |
| 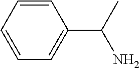 | 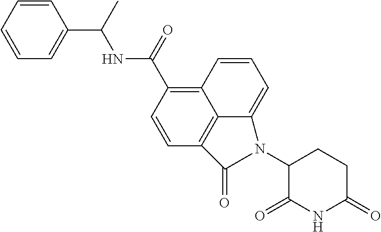 [SMILES: CC(NC(=O)c1ccc2c3c(cccc13)N(C1CCC(=O)NC1=O)C2=O)c1ccccc1] | Compound 182 | 428.0 |
| 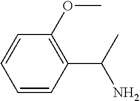 | 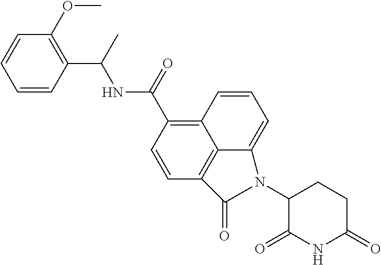 [SMILES: COc1ccccc1C(C)NC(=O)c1ccc2c3c(cccc13)N(C1CCC(=O)NC1=O)C2=O] | Compound 183 | 458.0 |
| 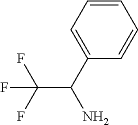 [SMILES: NC(c1ccccc1)C(F)(F)F] | 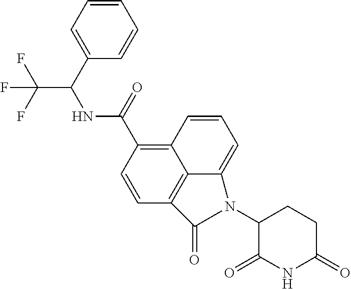 [SMILES: O=C1CCC(N2C(=O)c3ccc(C(=O)NC(c4ccccc4)C(F)(F)F)c4cccc2c34)C(=O)N1] | Compound 184 | 480.2 (ES−) |

### Example 92. 27-(2,6-dioxo-3-piperidyl)-20-oxo-N-(1-phenylethyl)-24,27-diazatricyclododeca-6,8(15),9(24),13(16),14(17)-pentaene-14-carboxamide (Compound 185)

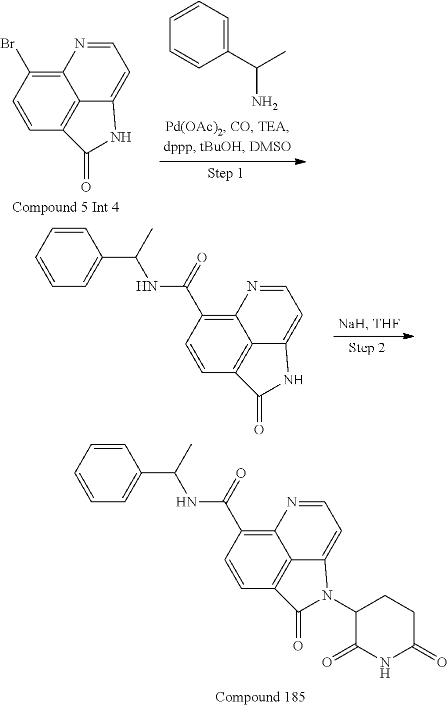

Step 1: To a stirred solution of 5-bromo-13$1\hat{ }{3}-broma-10-azatricyclododeca-(4),1(5),2(8),3(13),6-pentaen-9-one (300 mg, 952.50 μmol) and 1-phenylethan-1-amine (173 mg, 1.43 mmol) in a mixed solvent of HPLC grade t-BuOH (8 mL) and DMSO (0.8 mL), 3-diphenylphosphanylpropyl(diphenyl)phosphane (58.93 mg, 142.88 μmol) was added and resulting solution was degassed with argon for 15 min. To this well degassed solution were added triethylamine (0.275 mL, 1.91 mmol) and diacetoxypalladium (32.08 mg, 142.88 μmol) and resulting reaction mixture was heated at 100° C. in 60 psi of CO gas for 12 hr. After completion of reaction (as evidenced from LC MS), the reaction mixture was diluted with ethyl acetate (50 mL) and washed with water and brine several time. Organic phase was separated, dried over sodium sulfate and concentrated under reduced pressure. The crude reaction mass was purified by column chromatography in 100-200 silica in 10-15% EtOAc in hexane to afford 16-oxo-N-(1-phenylethyl)-19,20-diazatricyclododeca-6(11),7(12),8(13),9(19),14-pentaene-12-carboxamide (40 mg, 113.44 μmol, 11.91% yield) as a colorless gum. LCMS (ES⁺): m/z 318.3 [M+H]

Step 2: To a cooled solution of 16-oxo-N-(1-phenylethyl)-19,20-diazatricyclododeca-6,8(13),9(19),11(14),12(15)-pentaene-12-carboxamide (40 mg, 126.05 μmol) in dry THE (5 mL), Sodium hydride (in oil dispersion) 60% dispersion in mineral oil (48.30 mg, 1.26 mmol) was added portion wise, maintaining the temp <5° C. Once the addition is over, the resultant mixture was stirred for 15 minutes at RT. Then the reaction mixture was again cooled to 0° C. and 3-bromopiperidine-2,6-dione (121.01 mg, 630.24 μmol) was added to it portion wise. After complete addition, resulting solution was heated at 70° C. 1 hr. After complete consumption of 16-oxo-N-(1-phenylethyl)-19,20-diazatricyclododeca-6,8(13),9(19),11(14),12(15)-pentaene-12-carboxamide (evidenced from TLC), the reaction mixture was cooled to 0° C. and quenched with the addition of ice-cold water (5 mL). Aqueous part was extracted with ethyl acetate (3×50 mL). Combined organics was separated, dried over anhydrous sodium sulfate, and concentrated under reduced pressure. Crude was purified by PREP TLC to afford 27-(2,6-dioxo-3-piperidyl)-20-oxo-N-(1-phenylethyl)-24,27-diazatricyclododeca-6,8(15),9(24),13(16),14(17)-pentaene-14-carboxamide Compound 185 (21 mg, 48.04 μmol, 38.11% yield) as yellow solid. ¹H NMR (400 MHz, DMSO-d₆) δ 11.20 (s, 1H), 11.06 (d, J=7.76 Hz, 1H), 9.01 (d, J=4.96 Hz, 1H), 8.63 (d, J=7.32 Hz, 1H), 8.25 (d, J=7.28 Hz, 1H), 7.47-7.45 (m, 2H), 7.36 (t, J=7.28 Hz, 3H), 7.26 (t, J=7.28 Hz, 1H), 5.50 (dd, J1=11.84, J2=3.48 Hz, 1H), 5.32-5.28 (m, 1H), 2.94 (m, 1H), 2.76-2.65 (m, 2H), 2.17-2.15 (m, 1H), 1.59 (d, J=6.88 Hz, 3H); LCMS (ES⁺): m/z 429.4 [M+H]+

### Example 93. Methyl 4-[[1-(2,6-dioxo-3-piperidyl)-2-oxo-benzo[cd]indol-3-yl]amino]benzoate (Compound 186)

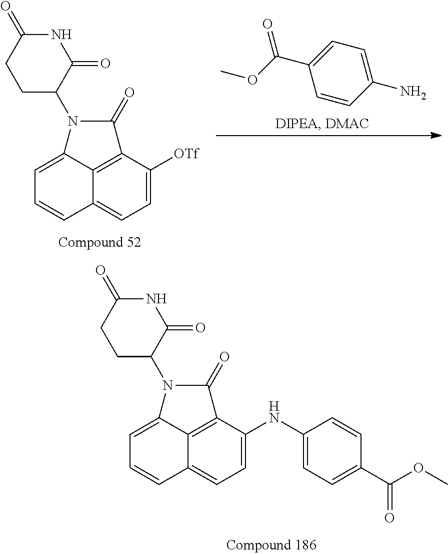 [SMILES: O=C1CCC(N2C(=O)c3c(OS(=O)(=O)C(F)(F)F)ccc4cccc2c34)C(=O)N1 ; COC(=O)c1ccc(N)cc1 ; COC(=O)c1ccc(Nc2ccc3cccc4c3c2C(=O)N4C2CCC(=O)NC2=O)cc1]

To well stirred solution of [1-(2,6-dioxo-3-piperidyl)-2-oxo-benzo[cd]indol-3-yl]trifluoromethanesulfonate (50 mg, 117 μmol, 1 eq) was added N-ethyl-N-isopropyl-propan-2-amine (30 mg, 41 μmol, 2 eq) and the reaction was stirred at room temperature for 1 h, methyl 4-aminobenzoate (21 mg, 140 μmol, 1.2 eq) was added into it and then the reaction mass was allowed to stir at 70° C. for 12 h. Crude LC-MS showed the desired compound formation. So, the crude reaction mass purified by reverse phase PREP HPLC purification to get methyl 4-[[1-(2,6-dioxo-3-piperidyl)-2-oxo-benzo[cd]indol-3-yl]amino]benzoate Compound 186 (12 mg, 0.027 mmol, yield 24%) LCMS (ESI): m/z 430.1 [M+H]⁺.

Using similar conditions, the products on the right were synthesized using the corresponding amines on the left.

| | | Compound | |
|---|---|---|---|
| Amine | Product | No. | LCMS |
| 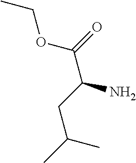 | 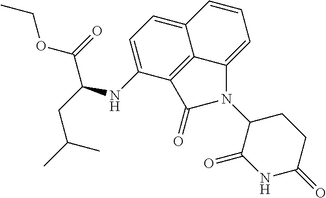 [SMILES: CCOC(=O)[C@H](CC(C)C)Nc1ccc2cccc3c2c1C(=O)N3C1CCC(=O)NC1=O] | Compound 187 | 438.2 |
| 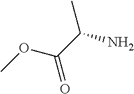 | 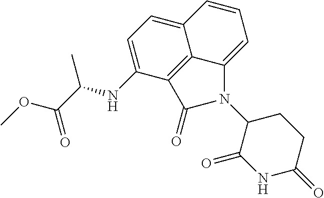 [SMILES: COC(=O)[C@H](C)Nc1ccc2cccc3c2c1C(=O)N3C1CCC(=O)NC1=O] | Compound 188 | 382.1 |
| 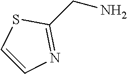 | 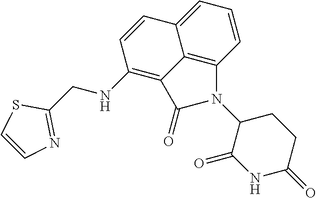 [SMILES: O=C1CCC(N2C(=O)c3c(NCc4nccs4)ccc4cccc2c34)C(=O)N1] | Compound 189 | 393.1 |
| 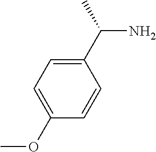 | 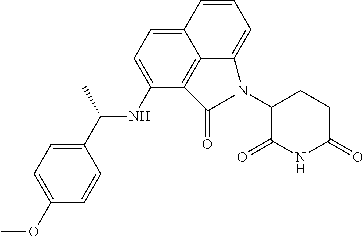 [SMILES: COc1ccc([C@H](C)Nc2ccc3cccc4c3c2C(=O)N4C2CCC(=O)NC2=O)cc1] | Compound 190 | 428.1 (ES−) |
| 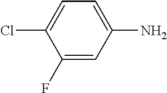 | 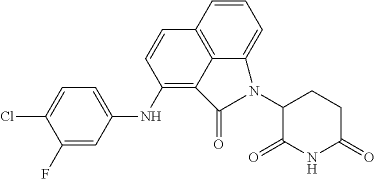 [SMILES: O=C1CCC(N2C(=O)c3c(Nc4ccc(Cl)c(F)c4)ccc4cccc2c34)C(=O)N1] | Compound 191 | 421.9 (ES−) |
| 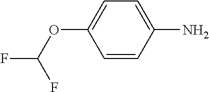 [SMILES: Nc1ccc(OC(F)F)cc1] | 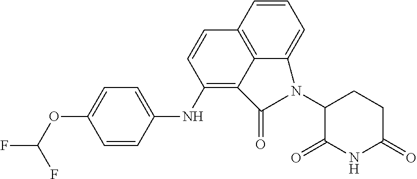 [SMILES: O=C1CCC(N2C(=O)c3c(Nc4ccc(OC(F)F)cc4)ccc4cccc2c34)C(=O)N1] | Compound 192 | 435.9 (ES−) |
| 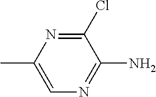 | 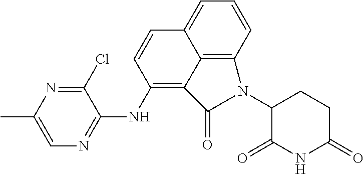 [SMILES: Cc1cnc(Nc2ccc3cccc4c3c2C(=O)N4C2CCC(=O)NC2=O)c(Cl)n1] | Compound 193 | 422.1 |
| 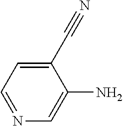 | 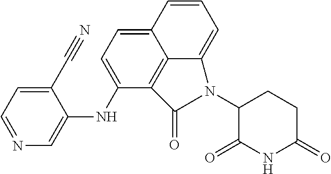 [SMILES: N#Cc1ccncc1Nc1ccc2cccc3c2c1C(=O)N3C1CCC(=O)NC1=O] | Compound 194 | 398.2 |
| 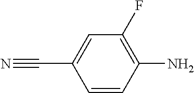 | 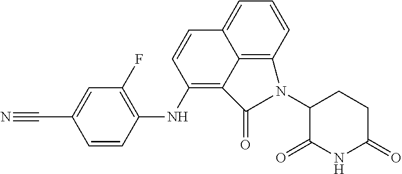 [SMILES: N#Cc1ccc(Nc2ccc3cccc4c3c2C(=O)N4C2CCC(=O)NC2=O)c(F)c1] | Compound 195 | 415.1 |

### Example 94. 3-[6-[[1-(1-chloro-4-methyl-4-piperidyl)pyrazol-4-yl]methyl]-2-oxo-benzo[cd]indol-1-yl]piperidine-2,6-dione (Compound 196)

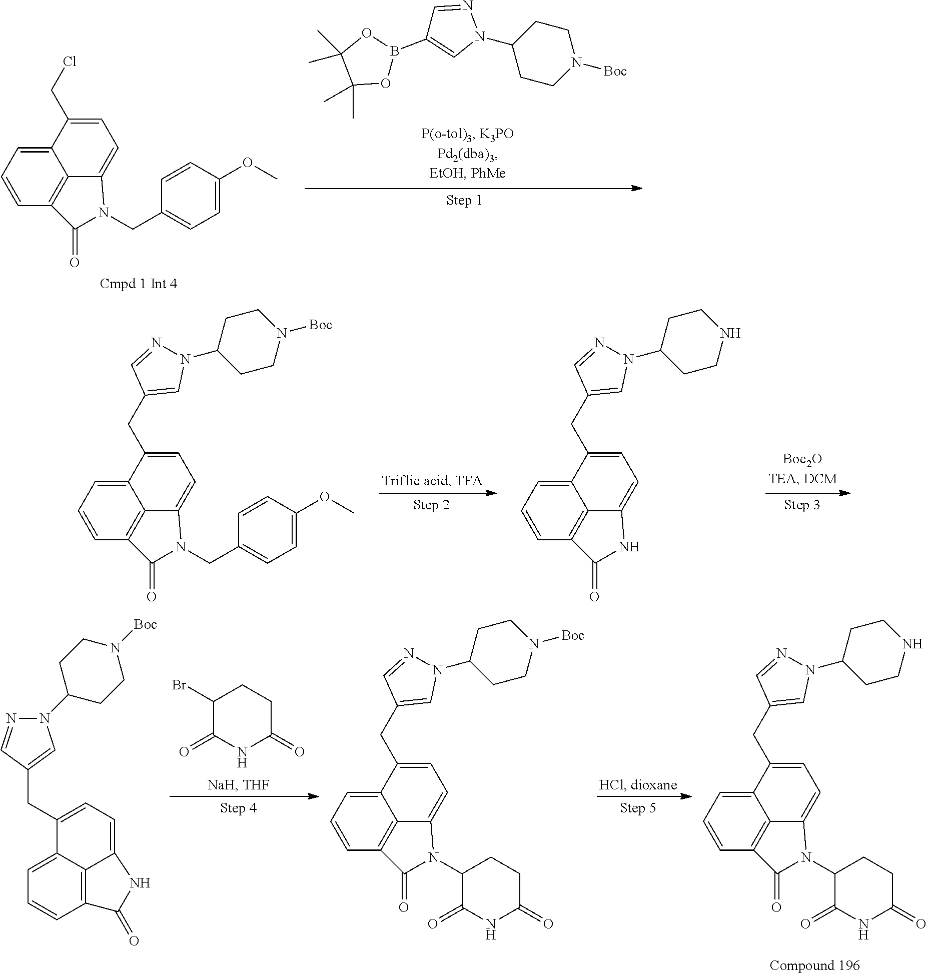

Step 1: To the stirred solution of 6-(chloromethyl)-1-[(4-methoxyphenyl)methyl]benzo[cd]indol-2-one (7.0 g, 20.72 mmol) and tert-butyl 4-[4-(4,4,5,5-tetramethyl-1,3,2-dioxaborolan-2-yl)pyrazol-1-yl]piperidine-1-carboxylate (10.16 g, 26.94 mmol) in a sealed tube in ethanol (12 mL) and toluene (24 mL) and 4 drops water were added tripotassium phosphate (11.00 g, 51.81 mmol). It was degassed with argon for 10 minutes. tris-o-tolylphosphane (1.26 g, 4.14 mmol) and Pd₂(dba)₃ (1.90 g, 2.07 mmol) were added to the reaction mixture. It was heated at 90° C. for 16 h. It was cooled to room temperature, filtered through celite, concentrated under reduced pressure. It was purified by column chromatography eluting at 50% ethyl acetate in hexane to afford tert-butyl 4-[4-[[1-[(4-methoxyphenyl)methyl]-2-oxo-benzo[cd]indol-6-yl]methyl]pyrazol-1-yl]piperidine-1-carboxylate (6.7 g, 11.03 mmol, 53.24% yield) as yellow solid. LCMS (ESI): m/z 553.4 [M+H]⁺.

Step 2: To the stirred solution of tert-butyl 4-[4-[[1-[(4-methoxyphenyl)methyl]-2-oxo-benzo[cd]indol-6-yl]methyl]pyrazol-1-yl]piperidine-1-carboxylate (6.6 g, 11.94 mmol) in TFA (20 mL) was added Trifluoromethanesulfonic acid (8.96 g, 59.71 mmol, 5.24 mL). It was stirred at RT for 16 h. It was concentrated under reduced pressure to afford 6-[[1-(4-piperidyl)pyrazol-4-yl]methyl]-1H-benzo[cd]indol-2-one trifluoroacetate (5.33 g, 10.86 mmol, 90.98% yield) as brown gum. LCMS (ESI): m/z 333.3 [M+H]⁺.

Step 3: To the stirred solution of 6-[[1-(4-piperidyl)pyrazol-4-yl]methyl]-1H-benzo[cd]indol-2-one (5.33 g, 16.03 mmol) in DCM (40 mL) was added triethyl amine (4.87 g, 48.10 mmol, 6.70 mL), followed by di-tert-butyl dicarbonate (3.50 g, 16.03 mmol, 3.68 mL). The reaction was stirred at RT for 16 h. It was concentrated under reduced pressure, diluted with water, extracted with ethyl acetate, washed with saturated sodium bicarbonate solution, brine, dried over sodium sulfate and concentrated under reduced pressure. Crude material was purified by combi flash eluting at 60% ethyl acetate in hexane to afford tert-butyl 4-[4-[(2-oxo-1H-benzo[cd]indol-6-yl)methyl]pyrazol-1-yl]piperidine-1-carboxylate (6.1 g, 13.51 mmol, 84.25% yield) as yellow solid. LCMS (ESI): m/z 433.5 [M+H]⁺.

Step 4: To the stirred solution of tert-butyl 4-[4-[(2-oxo-1H-benzo[cd]indol-6-yl)methyl]pyrazol-1-yl]piperidine-1-carboxylate (5 g, 11.56 mmol) in THE (70 mL) was added Sodium hydride (in oil dispersion) 60% dispersion in mineral oil (4.24 g, 110.75 mmol) at cold condition and the reaction mixture was stirred at room temperature for 10 minutes followed by the addition of 3-bromopiperidine-2,6-dione (11.10 g, 57.80 mmol) portion wise. It was then stirred at room temperature for 10 minutes and heated at 70° C. for 30 minutes. TLC was checked which showed almost complete consumption of the starting material and formation of the desired spot. The reaction mixture was diluted with ethyl acetate, washed with cold water and the organic fraction was separated. It was then dried over anhydrous sodium sulphate and evaporated under reduced pressure to obtain the crude which was washed with ether and pentane to afford tert-butyl 4-[4-[[1-(2,6-dioxo-3-piperidyl)-2-oxo-benzo[cd]indol-6-yl]methyl]pyrazol-1-yl]piperidine-1-carboxylate (3.8 g, 6.58 mmol, 56.96% yield) as yellow solid. LCMS (ESI): m/z 544.3 [M+H]⁺.

Step 5: To the stirred solution of tert-butyl 4-[4-[[1-(2,6-dioxo-3-piperidyl)-2-oxo-benzo[cd]indol-6-yl]methyl]pyrazol-1-yl]piperidine-1-carboxylate (3.8 g, 6.99 mmol) in Dioxane (10 mL) was added Hydrochloric acid in dioxane (6.99 mmol, 15 mL) and the reaction mixture was stirred at room temperature for 2 hours. TLC was checked which showed complete consumption of the starting material. The solvent in the reaction mixture was evaporated under reduced pressure to obtain a yellow solid which was washed with ether and pentane to afford 3-[6-[[1-(1-chloro-4-piperidyl)pyrazol-4-yl]methyl]-2-oxo-benzo[cd]indol-1-yl]piperidine-2,6-dione Compound 196 (3.3 g, 5.75 mmol, 82.27% yield) as yellow solid. LCMS (ESI): m/z 444.4 [M+H]⁺.

### Example 95. tert-butyl 4-[4-[[1-[(3S)-2,6-dioxo-3-piperidyl]-2-oxo-benzo[cd]indol-6-yl]methyl]pyrazol-1-yl]piperidine-1-carboxylate (Compound 197) and tert-butyl 4-[4-[[1-[(3R)-2,6-dioxo-3-piperidyl]-2-oxo-benzo[cd]indol-6-yl]methyl]pyrazol-1-yl]piperidine-1-carboxylate (Compound 198)

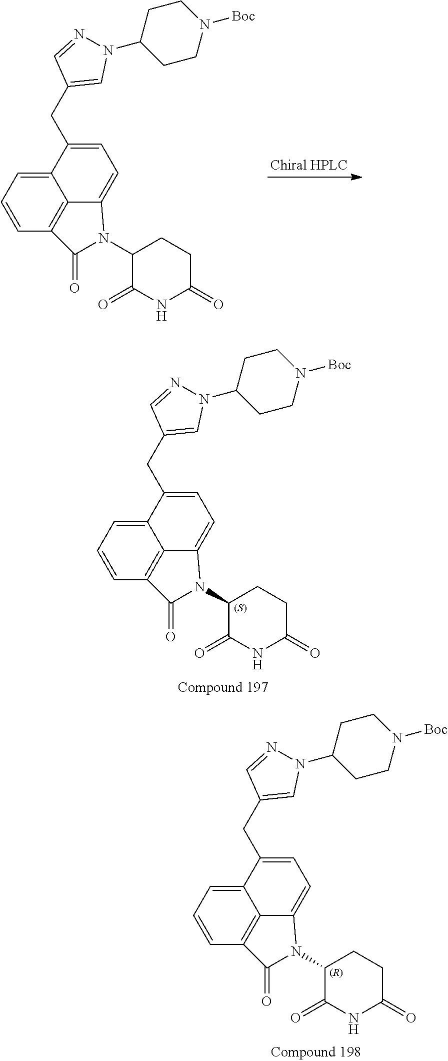

4-[4-[[1-[(3S)-2,6-dioxo-3-piperidyl]-2-oxo-benzo[cd]indol-6-yl]methyl]pyrazol-1-yl]piperidine-1-carboxylate Compound 197 and tert-butyl 4-[4-[[1-[(3R)-2,6-dioxo-3-piperidyl]-2-oxo-benzo[cd]indol-6-yl]methyl]pyrazol-1-yl]piperidine-1-carboxylate Compound 198 were obtained from Example 95 through step 4 by taking the crude product and separating the crude isomers by reverse phase chiral HPLC to afford tert-butyl 4-[4-[[1-[(3S)-2,6-dioxo-3-piperidyl]-2-oxo-benzo[cd]indol-6-yl]methyl]pyrazol-1-yl]piperidine-1-carboxylate (27.0 mg, 49.22 μmol, 2.66% yield) and tert-butyl 4-[4-[[1-[(3R)-2,6-dioxo-3-piperidyl]-2-oxo-benzo[cd]indol-6-yl]methyl]pyrazol-1-yl]piperidine-1-carboxylate (26.0 mg, 47.83 μmol, 2.59% yield) both as yellow solid. LCMS (ESI): m/z 344.5 [M+H]⁺.

### Example 96. 3-[6-[[1-(1-chloro-4-methyl-4-piperidyl)pyrazol-4-yl]methyl]-2-oxo-benzo[cd]indol-1-yl]piperidine-2,6-dione (Compound 199)

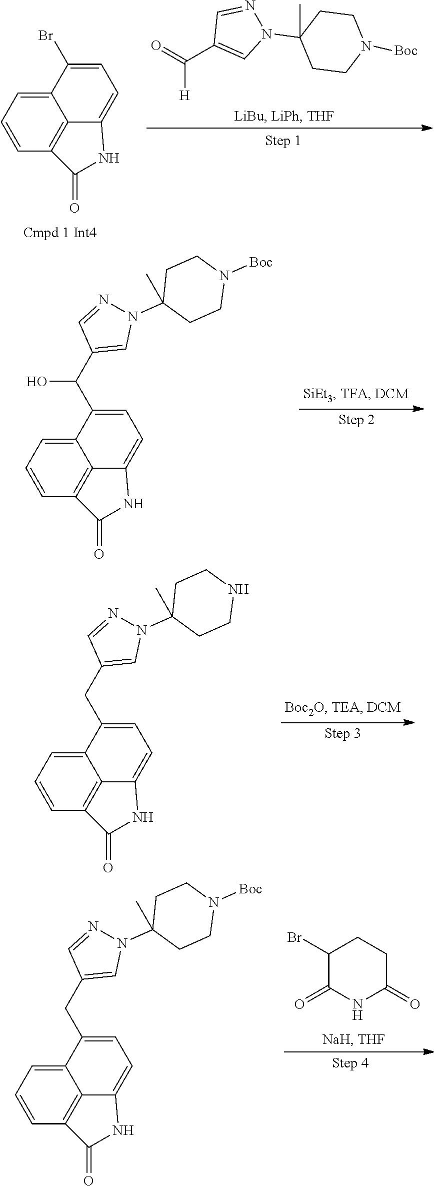

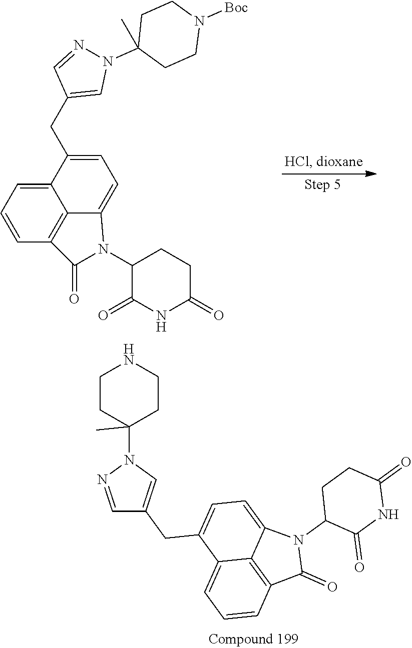 [SMILES: CC(C)(C)OC(=O)N1CCC(C)(n2cc(Cc3ccc4c5c(cccc35)C(=O)N4C3CCC(=O)NC3=O)cn2)CC1 ; CC1(n2cc(Cc3ccc4c5c(cccc35)C(=O)N4C3CCC(=O)NC3=O)cn2)CCNCC1]

Step 1: To the stirred solution of 6-bromo-1H-benzo[cd]indol-2-one (2.15 g, 8.67 mmol) in THE (25 mL) was added phenyllithium in di-n-butyl ether (1.8 M, 4.81 mL) at −78° C. and the reaction mixture was stirred at the same temperature for 1 hour followed by the addition of butyllithium (1.67 M, 5.71 mL) at −78° C. and after the addition was complete the temperature was allowed to increase to −40° C. and the reaction mixture was stirred at the same temperature for 30 minutes followed by the addition of tert-butyl 4-(4-formylpyrazol-1-yl)-4-methyl-piperidine-1-carboxylate (2.54 g, 8.67 mmol) in THE (25 mL) at −78° C. and then the reaction mixture was allowed to warm to room temperature and was continued for 16 hours. TLC was checked which showed formation of the desired spot. The reaction mixture was quenched with ammonium chloride solution, diluted with ethyl acetate, washed with water and the organic fraction was separated. It was then dried over anhydrous sodium sulphate and evaporated under reduced pressure to obtain the crude compound which was purified by flash chromatography using 0-5% MeOH-DCM to afford tert-butyl 4-[4-[hydroxy-(2-oxo-1H-benzo[cd]indol-6-yl)methyl]pyrazol-1-yl]-4-methyl-piperidine-1-carboxylate (1.5 g, 2.91 mmol, 33.53% yield) as brown solid. LCMS (ESI): m/z 363.1 [M+H-Boc]⁺.

Step 2: To the stirred solution of tert-butyl 4-[4-[hydroxy-(2-oxo-1H-benzo[cd]indol-6-yl)methyl]pyrazol-1-yl]-4-methyl-piperidine-1-carboxylate (1.5 g, 3.24 mmol) in DCE (8 mL) was added triethylsilane (1.51 g, 12.97 mmol, 2.07 mL), trifluoroacetic acid (2.96 g, 25.94 mmol, 2.00 mL) and the reaction mixture was heated at 80° C. for 2 hours in a sealed tube. TLC was checked which showed complete consumption of the starting material along with the formation of the desired spot. The solvent in the reaction mixture was evaporated under reduced pressure and triturated with ether to obtain [4-methyl-4-[4-[(2-oxo-1H-benzo[cd]indol-6-yl)methyl]pyrazol-1-yl]-1-piperidyl] 2,2,2-trifluoroacetate (1.4 g, 2.23 mmol, 68.69% yield) as crude which was used directly in the next step. LCMS (ESI): m/z 347.4 [M+H]⁺.

Step 3: To a stirred solution of [4-methyl-4-[4-[(2-oxo-1H-benzo[cd]indol-6-yl)methyl]pyrazol-1-yl]-1-piperidyl] 2,2,2-trifluoroacetate (1.49 g, 3.24 mmol) in DCM (10.0 mL) was added triethylamine (982.35 mg, 9.71 mmol, 1.35 mL) at 0° C. followed by the addition of di-tert-butyl dicarbonate (1.06 g, 4.85 mmol, 1.11 mL) and the reaction was stirred at room temperature for 16 hours. TLC was checked which showed complete consumption of the starting material along with the formation of the desired spot. The reaction mixture was diluted with ethyl acetate, washed with water and the organic fraction was separated. It was dried over anhydrous sodium sulphate and evaporated under reduced pressure to obtain the crude compound which was purified by flash chromatography using 0-5% MeOH-DCM to afford tert-butyl 4-methyl-4-[4-[(2-oxo-1H-benzo[cd]indol-6-yl)methyl]pyrazol-1-yl]piperidine-1-carboxylate (1.2 g, 2.45 mmol, 75.57% yield) as brown solid. LCMS (ESI): m/z 447.5 [M+H]⁺.

Step 4: To the stirred solution of tert-butyl 4-methyl-4-[4-[(2-oxo-1H-benzo[cd]indol-6-yl)methyl]pyrazol-1-yl]piperidine-1-carboxylate (1.2 g, 2.69 mmol) in THE (20 mL) was added Sodium hydride (60% dispersion in mineral oil) (1.03 g, 26.87 mmol) at cold condition and the reaction mixture was stirred at room temperature for 10 minutes followed by the addition of 3-bromopiperidine-2,6-dione (2.58 g, 13.44 mmol) portion wise. It was then stirred at room temperature for 10 minutes and heated at 70° C. for 30 minutes. TLC was checked which showed almost complete consumption of the starting material and formation of the desired spot. The reaction mixture was diluted with ethyl acetate, washed with cold water and the organic fraction was separated. It was then dried over anhydrous sodium sulphate and evaporated under reduced pressure to obtain the crude which was washed with ether and pentane to afford tert-butyl 4-[4-[[1-(2,6-dioxo-3-piperidyl)-2-oxo-benzo[cd]indol-6-yl]methyl]pyrazol-1-yl]-4-methyl-piperidine-1-carboxylate (1.1 g, 1.84 mmol, 68.63% yield) as yellow solid. LCMS (ESI): m/z 558.2 [M+H]⁺.

Step 5: To the stirred solution of tert-butyl 4-[4-[[1-(2,6-dioxo-3-piperidyl)-2-oxo-benzo[cd]indol-6-yl]methyl]pyrazol-1-yl]-4-methyl-piperidine-1-carboxylate (205.16 mg, 367.91 μmol) in Dioxane (1 mL) was added Hydrochloric acid in 1,4-dioxane (367.91 μmol, 6 mL) and the reaction mixture was stirred at room temperature for 2 hours. TLC was checked which showed complete consumption of the starting material. The solvent in the reaction mixture was evaporated under reduced pressure to obtain a yellow solid which was washed with ether and pentane to afford 3-[6-[[1-(1-chloro-4-methyl-4-piperidyl)pyrazol-4-yl]methyl]-2-oxo-benzo[cd]indol-1-yl]piperidine-2,6-dione Compound 199 (180.0 mg, 333.57 μmol, 90.67% yield) as yellow solid. LCMS (ESI):m/z 458.4 [M+H]⁺.

### Example 97. 1-[4-[4-[[1-(2,6-dioxo-3-piperidyl)-2-oxo-benzo[cd]indol-6-yl]methyl]pyrazol-1-yl]piperidine-1-carbonyl]cyclobutanecarbonitrile (Compound 200)

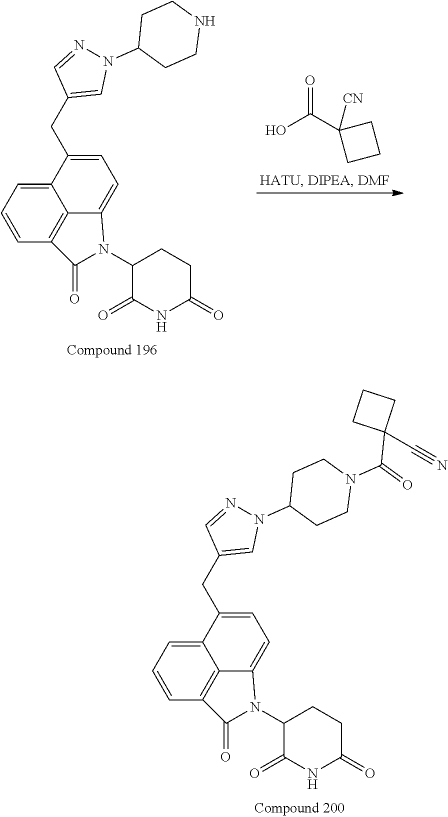 [SMILES: O=C1CCC(N2C(=O)c3cccc4c(Cc5cnn(C6CCNCC6)c5)ccc2c34)C(=O)N1 ; N#CC1(C(=O)O)CCC1 ; N#CC1(C(=O)N2CCC(n3cc(Cc4ccc5c6c(cccc46)C(=O)N5C4CCC(=O)NC4=O)cn3)CC2)CCC1]

Step 1: To the stirred solution of 3-[6-[[1-(1-chloro-4-piperidyl)pyrazol-4-yl]methyl]-2-oxo-benzo[cd]indol-1-yl]piperidine-2,6-dione (215.0 mg, 448 μmol, 1 eq) in DMF (1 mL) was added N,N-Diisopropylethylamine (231.58 mg, 1.79 mmol, 312.10 μL, 4 eq) in cold condition followed by the addition 1-cyanocyclobutanecarboxylic acid (56.05 mg, 448 μmol, 1 eq) and HATU (255.49 mg, 672 μmol, 1.5 eq) and the reaction was continued at room temperature for 16 hours. TLC was checked which showed formation of the desired spot. The reaction mixture was diluted with ethyl acetate, washed with sodium bicarbonate solution, water and the organic fraction was separated. It was dried over anhydrous sodium sulphate and evaporated under reduced pressure to obtain the crude which was purified by preparative TLC plate method developing the plate in 3% MeOH-DCM to afford 1-[4-[4-[[1-(2,6-dioxo-3-piperidyl)-2-oxo-benzo[cd]indol-6-yl]methyl]pyrazol-1-yl]piperidine-1-carbonyl]cyclobutanecarbonitrile Compound 200 (145.0 mg, 262.63 μmol, 58.63% yield) as yellow solid. LCMS (ESI): m/z 551.2 [M+H]⁺.

Using similar conditions, the products on the right were synthesized using the corresponding acids on the left.

| | | Compound | |
|---|---|---|---|
| Acid | Product | No. | LCMS |
| 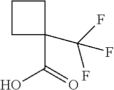 [SMILES: O=C(O)C1(C(F)(F)F)CCC1] | 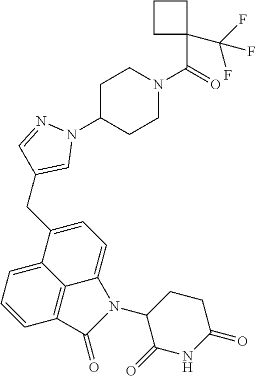 [SMILES: O=C1CCC(N2C(=O)c3cccc4c(Cc5cnn(C6CCN(C(=O)C7(C(F)(F)F)CCC7)CC6)c5)ccc2c34)C(=O)N1] | Compound 201 | 594.5 |
| 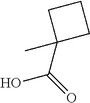 | 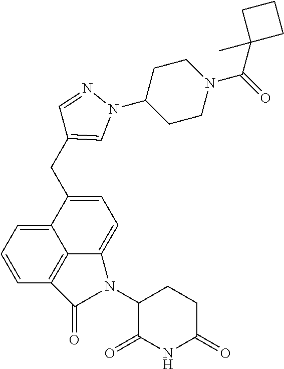 [SMILES: CC1(C(=O)N2CCC(n3cc(Cc4ccc5c6c(cccc46)C(=O)N5C4CCC(=O)NC4=O)cn3)CC2)CCC1] | Compound 202 | 540.6 |
| 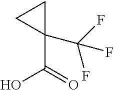 | 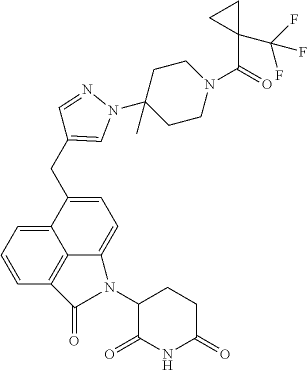 [SMILES: CC1(n2cc(Cc3ccc4c5c(cccc35)C(=O)N4C3CCC(=O)NC3=O)cn2)CCN(C(=O)C2(C(F)(F)F)CC2)CC1] | Compound 203 | 594.2 |
| 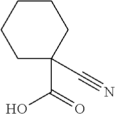 [SMILES: N#CC1(C(=O)O)CCCCC1] | 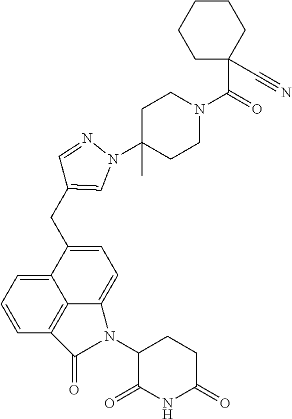 [SMILES: CC1(n2cc(Cc3ccc4c5c(cccc35)C(=O)N4C3CCC(=O)NC3=O)cn2)CCN(C(=O)C2(C#N)CCCCC2)CC1] | Compound 204 | 593.3 |
| 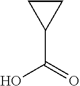 | 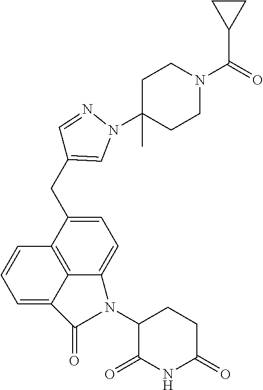 [SMILES: CC1(n2cc(Cc3ccc4c5c(cccc35)C(=O)N4C3CCC(=O)NC3=O)cn2)CCN(C(=O)C2CC2)CC1] | Compound 205 | 526.2 |

### Example 98. (3S)-3-[6-[[1-[1-(1-methylcyclobutanecarbonyl)-4-piperidyl]pyrazol-4-yl]methyl]-2-oxo-benzo[cd]indol-1-yl]piperidine-2,6-dione (Compound 206) and (3R)-3-[6-[[1-[1-(1-methylcyclobutanecarbonyl)-4-piperidyl]pyrazol-4-yl]methyl]-2-oxo-benzo[cd]indol-1-yl]piperidine-2,6-dione (Compound 207)

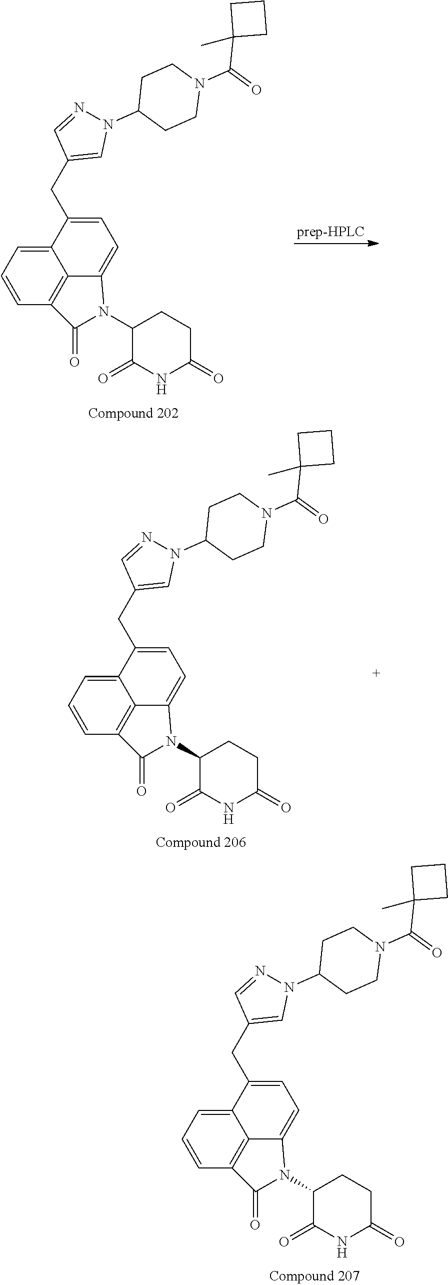 [SMILES: CC1(C(=O)N2CCC(n3cc(Cc4ccc5c6c(cccc46)C(=O)N5C4CCC(=O)NC4=O)cn3)CC2)CCC1 ; CC1(C(=O)N2CCC(n3cc(Cc4ccc5c6c(cccc46)C(=O)N5[C@H]4CCC(=O)NC4=O)cn3)CC2)CCC1 ; CC1(C(=O)N2CCC(n3cc(Cc4ccc5c6c(cccc46)C(=O)N5[C@@H]4CCC(=O)NC4=O)cn3)CC2)CCC1]

The product was submitted to preparative HPLC for separation of the isomers. From Prep HPLC two fractions were collected and we got (3S)-3-[6-[[1-[1-(1-methylcyclobutanecarbonyl)-4-piperidyl]pyrazol-4-yl]methyl]-2-oxo-benzo[cd]indol-1-yl]piperidine-2,6-dione (40.0 mg, 73.78 μmol, 39.82% yield) as yellow solid and (3R)-3-[6-[[1-[1-(1-methylcyclobutanecarbonyl)-4-piperidyl]pyrazol-4-yl]methyl]-2-oxo-benzo[cd]indol-1-yl]piperidine-2,6-dione (40.0 mg, 73.19 μmol, 39.50% yield) as yellow solid. The isomers were separated and the method for prep HPLC as given below: Solvent Name A: CAN, Solvent Name B: 0.1% TFA COLUMN NAME: REFLECT I CELLULOSE C 5μ, (25 cm×21.1 mm), Time: 42 minutes, Flow rate: 16 mL/min. LCMS (ESI): m/z 540.5 [M+H]⁺.

### Example 99. -[6-[[1-[1-(3-fluoro-2-pyridyl)-4-methyl-4-piperidyl]pyrazol-4-yl]methyl]-2-oxo-benzo[cd]indol-1-yl]piperidine-2,6-dione (Compound 208)

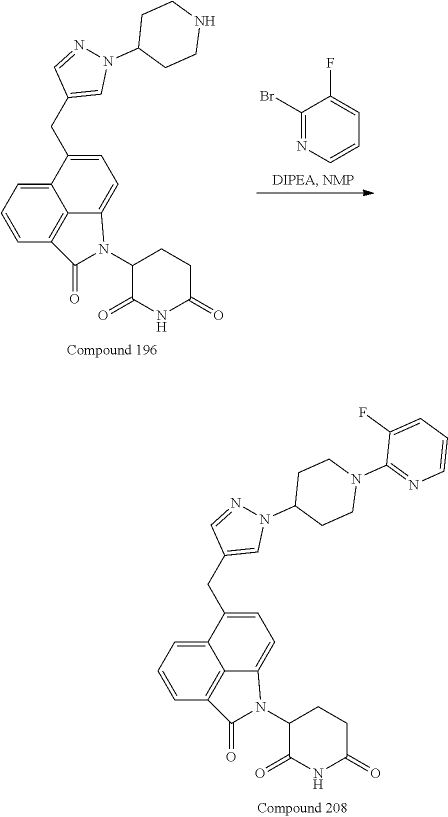 [SMILES: O=C1CCC(N2C(=O)c3cccc4c(Cc5cnn(C6CCNCC6)c5)ccc2c34)C(=O)N1 ; Fc1cccnc1Br ; O=C1CCC(N2C(=O)c3cccc4c(Cc5cnn(C6CCN(c7ncccc7F)CC6)c5)ccc2c34)C(=O)N1]

To the well degassed solution of 3-[6-[[1-(4-methyl-4-piperidyl)pyrazol-4-yl]methyl]-2-oxo-benzo[cd]indol-1-yl]piperidine-2,6-dione; hydrochloride (200 mg, 404.87 μmol) in NMP (2.0 mL) N,N-Diisopropylethylamine (313.95 mg, 2.43 mmol, 423.12 μL) was added followed by 2-bromo-3-fluoro-pyridine (285.01 mg, 1.62 mmol, 163.80 μL). Resulting solution was then heated at 110° C. for 12 hr. After completion of reaction as evidenced from LC MS. RM was cooled to RT and ice cooled water was added to it. Aqueous part was extracted with ethyl acetate (3×30 mL). Organic phase was separated, dried over sodium sulfate and concentrated. Crude residue was purified by Column chromatography followed by PREP TLC (40% Ethyl acetate in DCM) to afford 3-[6-[[1-[1-(3-fluoro-2-pyridyl)-4-methyl-4-piperidyl]pyrazol-4-yl]methyl]-2-oxo-benzo[cd]indol-1-yl]piperidine-2,6-dione Compound 208 (112 mg, 190.70 μmol, 47.10% yield). LCMS (ESI): m/z 553.4 [M+H]⁺.

### Example 100. 3-(5-indolin-1-yl-2-oxo-benzo[cd]indol-1-yl)piperidine-2,6-dione (Compound 209)

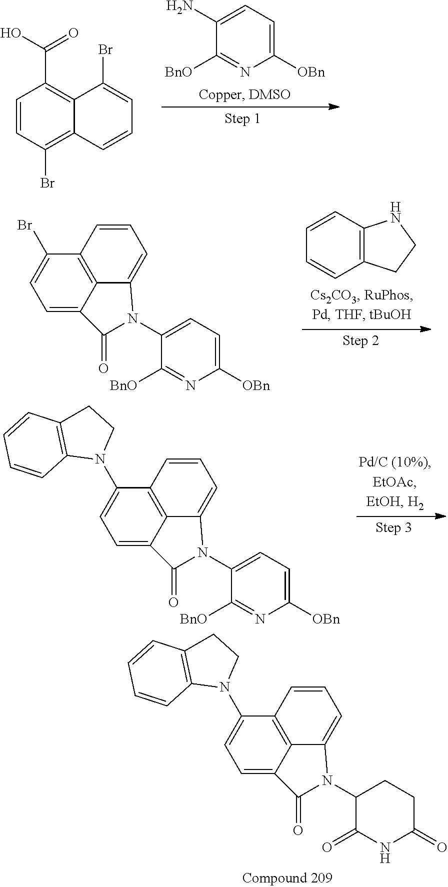

Step 1: To a stirred solution of 4,8-dibromonaphthalene-1-carboxylic acid (25 g, 75.76 mmol) in DMSO (200 mL) was added 2,6-dibenzyloxypyridin-3-amine (18.57 g, 60.61 mmol) and Copper (1.25 g, 19.70 mmol) at room temperature, then reaction mass was heated 90° C. over night. The reaction was monitored by TLC. The reaction was diluted with cold water, extracted with ethyl acetate. Organic layer was washed with brine dried over sodium sulphate concentrated under reduced pressure. Crude was purified by column chromatography eluted at 5% EA/Hexanes afforded 5-bromo-1-(2,6-dibenzyloxy-3-pyridyl)benzo[cd]indol-2-one (10 g, 13.77 mmol, 18.17% yield) light brown solid. LCMS (ESI): m/z 539.0 [M+H]⁺.

Step 2: To a stirred solution of 5-bromo-1-(2,6-dibenzyloxy-3-pyridyl)benzo[cd]indol-2-one (1 eq), indoline (250 mg, 1 eq), Cesium carbonate (2 eq) in THF (2 mL) and tert Butanol (2 mL) was added and the reaction mixture was degassed with argon. Then (1E,4E)-1,5-diphenylpenta-1,4-dien-3-one palladium (0.1 eq) and Ruphos (0.2 eq) were added, and the reaction mixture was stirred at 90° C. for 16 h. Reaction were monitored by LCMS. After completion the reaction mixture was filtered off and the filtrated was concentrated. The crude thus obtained was purified by column chromatography in combiflash column in 0-100% EtOAc in Hexane. To generate of 1-(2,6-dibenzyloxy-3-pyridyl)-5-indolin-1-yl-benzo[cd]indol-2-one (105 mg, 0.182 mmol, 39% yield) LCMS (ESI): m/z 576.2 [M+H]⁺.

Step 3: To the well degassed stirred solution of crude 1-(2,6-dibenzyloxy-3-pyridyl)-5-indolin-1-yl-benzo[cd]indol-2-one (100 mg, 1 eq) in ethyl acetate, 99.9% (5 mL) and ethanol (5 mL), palladium, 1000 on carbon, Type 487, dry (10 eq) was added and hydrogenated under balloon pressure for 16 hr. After completion of reaction, reaction mixture was filtered through cartridge filter. Filtrate was evaporated and crude was purified by reverse phase HPLC to afford 3-(5-indolin-1-yl-2-oxo-benzo[cd]indol-1-yl)piperidine-2,6-dione Compound 209 (22 mg, 0.055 mmol, 32% yield) LCMS (ESI): m/z 398.1 [M+H]⁺.

Using similar conditions, the products on the right were synthesized using the corresponding amines on the left.

| Amine | Product | Compound No. | LCMS Step 2 |
|---|---|---|---|
| 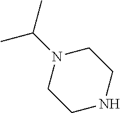 | 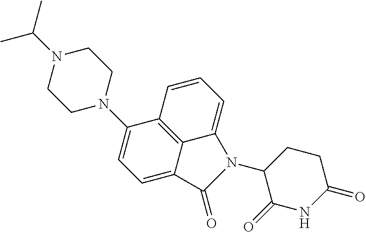 [SMILES: CC(C)N1CCN(c2ccc3c4c(cccc24)N(C2CCC(=O)NC2=O)C3=O)CC1] | Compound 210 | 407.3 |
| 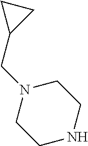 | 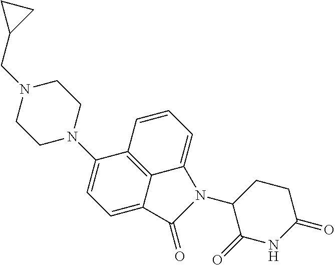 [SMILES: O=C1CCC(N2C(=O)c3ccc(N4CCN(CC5CC5)CC4)c4cccc2c34)C(=O)N1] | Compound 211 | 419.4 |
| 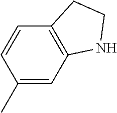 | 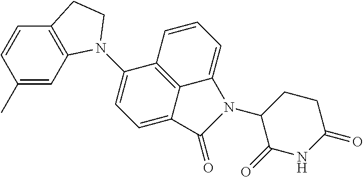 [SMILES: Cc1ccc2c(c1)N(c1ccc3c4c(cccc14)N(C1CCC(=O)NC1=O)C3=O)CC2] | Compound 212 | 412.3 |
| 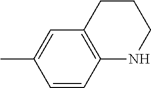 | 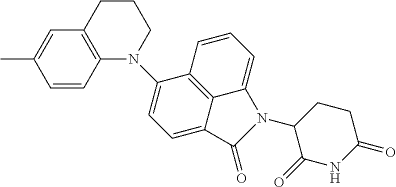 [SMILES: Cc1ccc2c(c1)CCCN2c1ccc2c3c(cccc13)N(C1CCC(=O)NC1=O)C2=O] | Compound 213 | 426.3 |
| 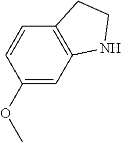 | 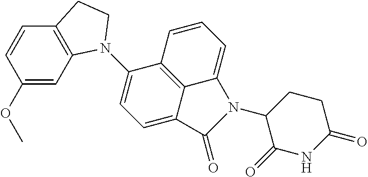 [SMILES: COc1ccc2c(c1)N(c1ccc3c4c(cccc14)N(C1CCC(=O)NC1=O)C3=O)CC2] | Compound 214 | 428.1 |
| 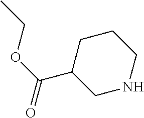 | 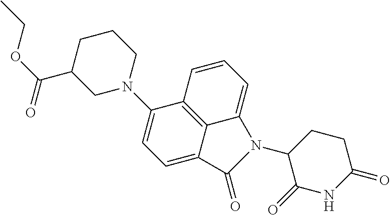 [SMILES: CCOC(=O)C1CCCN(c2ccc3c4c(cccc24)N(C2CCC(=O)NC2=O)C3=O)C1] | Compound 215 | 436.1 |
| 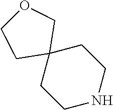 | 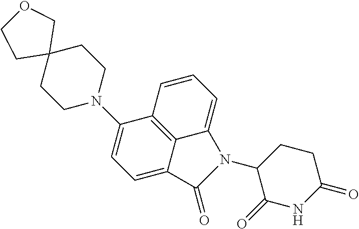 [SMILES: O=C1CCC(N2C(=O)c3ccc(N4CCC5(CCOC5)CC4)c4cccc2c34)C(=O)N1] | Compound 216 | 420.1 |
| 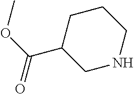 | 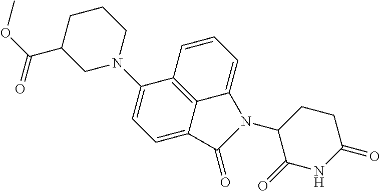 [SMILES: COC(=O)C1CCCN(c2ccc3c4c(cccc24)N(C2CCC(=O)NC2=O)C3=O)C1] | Compound 217 | 435.9 |

### Example 101. 3-[5-(4-methylpiperazin-1-yl)-2-oxo-benzo[cd]indol-1-yl]piperidine-2,6 (Compound 218)

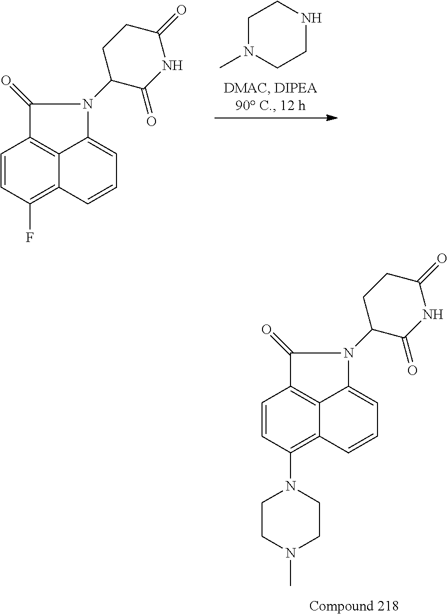

To the stirred solution of 1-methylpiperazine (20.15 mg, 201.16 μmol, 22.31 μL) (20.15 mg, 201.16 μmol, 22.31 μL) in HPLC grade DMAC (0.5 mL), DIPEA (21.67 mg, 167.63 μmol, 29.20 μL) was added and stirred for 30 min followed by the addition of 3-(5-fluoro-2-oxo-benzo[cd]indol-1-yl)piperidine-2,6-dione (50 mg, 167.63 μmol). Resulting solution was further heated at 90° C. for 12 hr. After completion of reaction (as evidenced from LCMS), ice cooled water (10 mL) was added to the reaction mixture and extracted with ethyl acetate (3×25 mL). The organic layer was separated, dried over anhydrous sodium sulfate, filtered and evaporated under reduced pressure to get crude residue which was purified by PREP-TLC to afford 3-[5-(4-methylpiperazin-1-yl)-2-oxo-benzo[cd]indol-1-yl]piperidine-2,6-dione Compound 218 (37 mg, 97.77 μmol, 58.33% yield) as white solid. ¹H NMR (400 MHz, DMSO-d₆) δ 11.09 (s, 1H), 7.95 (d, J=7.72 Hz, 1H), 7.60 (d, J=8.64 Hz, 1H), 7.43 (t, J=7.36 Hz, 1H), 7.16 (d, J=7.72 Hz, 1H), 7.07 (d, J=7.12 Hz, 1H), 5.40 (dd, J=12.48, 5.12 Hz, 1H), 2.96-2.93 (m, 1H), 2.78-2.71 (m, 1H), 2.62 (br s, 5H), 2.49 (br s, 4H), 2.18 (s, 3H), 1.98 (m, 1H); LCMS (ESI): m/z 379.3 [M+H]⁺.

Using similar conditions, the products on the right were synthesized using the corresponding amines on the left.

| Amine | Product | Compound No. | LCMS Step 2 |
|---|---|---|---|
| 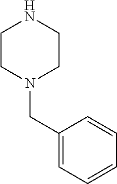 [SMILES: c1ccc(CN2CCNCC2)cc1] | 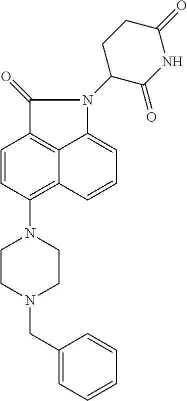 [SMILES: O=C1CCC(N2C(=O)c3ccc(N4CCN(Cc5ccccc5)CC4)c4cccc2c34)C(=O)N1] | Compound 219 | 455.4 |
| 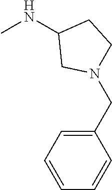 | 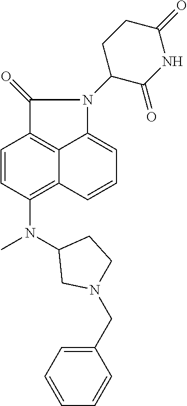 [SMILES: CN(c1ccc2c3c(cccc13)N(C1CCC(=O)NC1=O)C2=O)C1CCN(Cc2ccccc2)C1] | Compound 220 | 469.3 |

### Example 102. Synthesis of 2-(2,6-dioxo-3-piperidyl)-2,11-diazatricyclo[6.3.1.04, 12]dodeca-1(12),4,6,8-tetraene-3,10-dione (Compound 221)

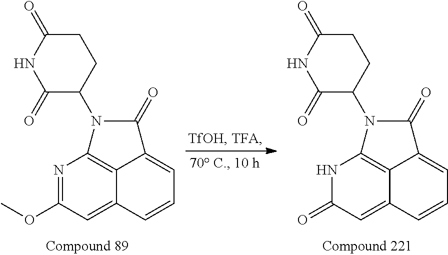 [SMILES: COc1cc2cccc3c2c(n1)N(C1CCC(=O)NC1=O)C3=O ; O=C1CCC(N2C(=O)c3cccc4cc(=O)[nH]c2c34)C(=O)N1]

To a solution of 3-(10-Methoxy-3-oxo-2,11-diazatricyclo[6.3.1.04, 12]dodeca-1(12),4,6,8,10-pentaen-2-yl)piperidine-2,6-dione (25 mg, 80.31 μmol) in trifluoroacetic acid (1 mL), was added triflic acid (18.08 mg, 120.47 μmol, 10.58 μL). The resulting mixture was heated at 90° C. for 8 h. The reaction mixture was concentrated under reduced pressure to give the residue which was dissolved in dichloromethane and methanol (9:1 ratio, 4 mL) and neutralized using Amberlyst® A21 free base resin. The resin was filtered, and the filtrate was concentrated under reduced pressure to give the crude product. The crude product was purified by preparative HPLC [Column: X select C18 (250×19) mm, 5 microns; Mobile phase: A: 0.1% HCOOH in water, B: Acetonitrile] to give 2-(2,6-dioxo-3-piperidyl)-2,11-diazatricyclo[6.3.1.04, 12]dodeca-1(12),4,6,8-tetraene-3,10-dione Compound 221 (5.0 mg, 16.69 μmol, 20.79% yield) as a yellow solid. LCMS (ES⁺): m/z 298.2 [M+H]⁺. ¹H NMR (400 MHz, DMSO-d₆): δ 11.15 (s, 1H), 10.95 (s, 1H), 8.00-7.96 (m, 1H), 7.87-7.83 (m, 2H), 6.55 (s, 1H), 5.37-5.33 (m, 1H), 3.03-2.88 (m, 2H), 2.70-2.66 (m, 1H), 2.18-2.12 (m, 1H) ppm.

### Example 103. Synthesis of 3-(10-methoxy-3-oxo-2,9-diazatricyclo[6.3.1.04, 12]dodeca-1(11),4,6,8(12),9-pentaen-2-yl)piperidine-2,6-dione (Compound 222) and 2-(2,6-dioxo-3-piperidyl)-2,9-diazatricyclo[6.3.1.04, 12]dodeca-1(11),4,6,8(12)-tetraene-3,10-dione (Compound 223)

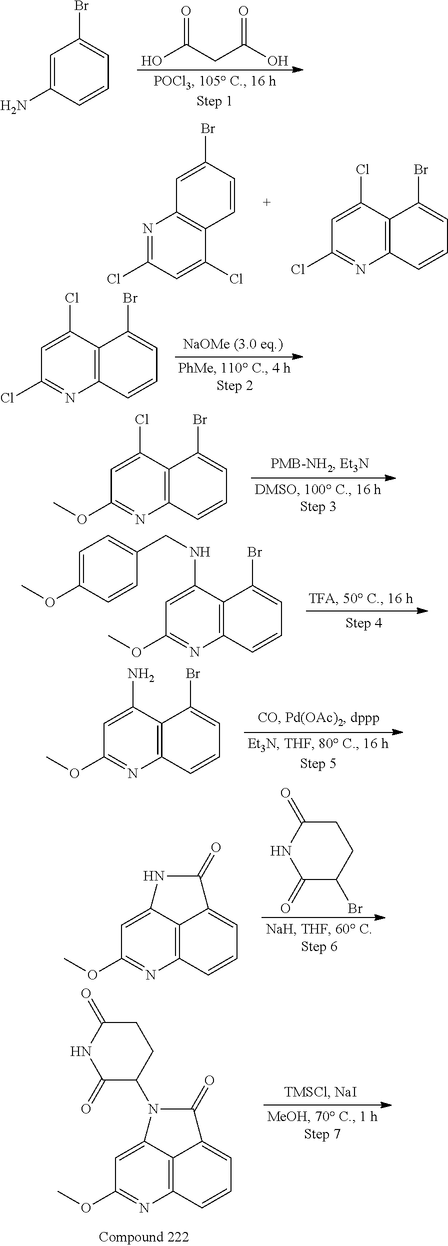

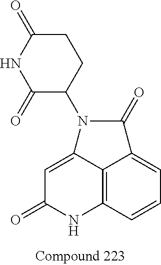 [SMILES: O=C1CCC(N2C(=O)c3cccc4[nH]c(=O)cc2c34)C(=O)N1]

### Step 1:

3-Bromoaniline (1 g, 5.81 mmol, 632.91 μL) was taken in phosphorous oxychloride (8 mL), was added malonic acid (604.93 mg, 5.81 mmol). The resulting mixture was heated at 105° C. for 16 h. The reaction mixture was concentrated under reduced pressure to give the residue. The residue was treated with cold water, neutralized using 10% aqueous sodium hydroxide solution and extracted with ethyl acetate (3×150 mL). The combined organics were washed with brine solution (100 mL) and dried over anhydrous sodium sulfate. The solution was filtered and concentrated under reduced pressure to give the crude product. The crude product was purified by flash chromatography (silica gel, 230-400 mesh) eluted with 4-5% ethyl acetate in petroleum ether to give late eluting 5-bromo-2,4-dichloro-quinoline (115 mg, 396.56 μmol, 6.82% yield) as an off-white solid. LCMS (ES⁺): m/z 279.8 [M+H]⁺. along with first eluting 7-bromo-2,4-dichloroquinoline (20 mg, 1.20% yield) as an off-white solid. LCMS (ES⁺): m/z 278.0 [M+H]⁺.

### Step 2:

To a solution of 5-bromo-2,4-dichloro-quinoline (520 mg, 1.88 mmol) in toluene (10 mL), was added sodium methoxide (304.31 mg, 5.63 mmol, 314.05 μL). The resulting mixture was heated at 100° C. for 6 h. The reaction mixture was treated water (20 mL) and extracted with ethyl acetate (2×50 mL). The combined organics were washed with brine solution (20 mL) and dried over anhydrous sodium sulfate. The solution was filtered and concentrated under reduced pressure to give the crude product. The crude product was purified by flash chromatography (silica gel, 230-400 mesh) eluted with 5-10% ethyl acetate in petroleum ether to give 5-bromo-4-chloro-2-methoxy-quinoline (430 mg, 1.29 mmol, 68.77% yield) as an off-white solid. LCMS (ES⁺): m/z 273.9 [M+H]⁺.

### Step 3:

To a solution of 5-bromo-4-chloro-2-methoxy-quinoline (420 mg, 1.54 mmol) in DMSO (7 mL), were added triethylamine (467.84 mg, 4.62 mmol, 644.41 μL) and 4-methoxybenzylamine (317.12 mg, 2.31 mmol, 302.02 μL). The resulting mixture was heated at 120° C. for 16 h. The reaction mixture was treated with water (20 mL) and extracted with ethyl acetate (2×40 mL). The combined organics were washed with brine solution (20 mL) and dried over anhydrous sodium sulfate. The solution was filtered, and the filtrate was concentrated under reduced pressure to give the crude product. The crude product was purified by flash chromatography (silica gel, 230-400 mesh) eluted with 20% ethyl acetate in petroleum ether to give 5-bromo-2-methoxy-N-[(4-methoxyphenyl)methyl]quinolin-4-amine (400 mg, 828.63 μmol, 53.77% yield) as an off-white solid. LCMS (ES⁺): m/z 373.0 [M+H]⁺.

### Step 4:

5-Bromo-2-methoxy-N-[(4-methoxyphenyl)methyl]quinolin-4-amine (400 mg, 1.07 mmol) was taken in trifluoroacetic acid (5 mL), and the resulting mixture was heated at 50° C. for 16 h. The reaction mixture was concentrated under reduced pressure to give the residue. The crude residue was triturated with dichloromethane, the precipitated solid was filtered and dried under vacuum to give 5-bromo-2-methoxy-quinolin-4-amine (340 mg, 977.17 μmol, 91.18% yield) as a brown solid. LCMS (ES⁺): m/z 252.9 [M+H]⁺. The crude product was taken to next step without purification.

### Step 5:

To a solution of 5-bromo-2-methoxy-quinolin-4-amine (340 mg, 1.34 mmol) in THE (20 mL) were added palladium(II) acetate (150.80 mg, 671.68 μmol), 1,3-bis(diphenylphosphino)propane (166.22 mg, 403.01 μmol) and triethylamine (135.94 mg, 1.34 mmol, 187.24 μL). The resulting mixture was heated at 85° C. in an atmosphere of carbon monoxide (5.5 kg/cm²) for 16 h. The reaction mixture was filtered through a pad of celite and the filtrate was concentrated under reduced pressure to give the crude product. The crude product was purified by flash chromatography (silica gel, 230-400 mesh) eluted with 25% ethyl acetate in petroleum ether to give 10-methoxy-2,9-diazatricyclo[6.3.1.04, 12]dodeca-1(11),4,6,8(12),9-pentaen-3-one (150 mg, 744.41 μmol, 55.41% yield) as a yellow solid. LCMS (ES⁺): m/z 201.0 [M+H]⁺.

### Step 6:

To a solution of 10-methoxy-2,9-diazatricyclo[6.3.1.04, 12]dodeca-1(11),4,6,8(12),9-pentaen-3-one (50.00 mg, 249.76 μmol) in THE (2 mL), cooled to 0° C., was added sodium hydride (60% dispersion in mineral oil, 57.42 mg, 1.50 mmol) and the resulting mixture was stirred room temperature for 30 min. The mixture was cooled to 0° C., was added 3-bromopiperidine-2,6-dione (119.89 mg, 624.40 μmol) in THF (1 mL) dropwise. The resulting mixture was heated at 65° C. for 5 h. The crude mixture was cooled to 0° C., treated with saturated ammonium chloride solution slowly and extracted with ethyl acetate (2×15 mL). The combined organics were washed with brine solution (10 mL) and dried over anhydrous sodium sulfate. The solution was filtered and concentrated under reduced pressure to give the crude product. The crude product was purified by flash chromatography (silica gel, 230-400 mesh) using 0-100% ethyl acetate in petroleum ether while the desired compound eluted at 50-60% ethyl acetate in petroleum ether to give 3-(10-methoxy-3-oxo-2,9-diazatricyclo[6.3.1.04, 12]dodeca-1(11),4,6,8(12),9-pentaen-2-yl)piperidine-2,6-dione Compound 222 (30 mg, 93.23 μmol, 37.33% yield) as an off-white solid. LCMS (ES⁺): m/z 312.2 [M+H]⁺. ¹H NMR (400 MHz, DMSO-d₆): δ 11.15 (s, 1H), 8.02 (dd, J=7.6, 0.8 Hz, 1H), 7.94-7.87 (m, 2H), 6.78 (s, 1H), 5.46-5.41 (m, 1H), 4.02 (s, 3H), 2.96-2.91 (m, 1H), 2.89-2.81 (m, 1H), 2.79-2.66 (m, 1H), 2.13-2.08 (m, 1H) ppm.

### Step 7:

To a solution of 3-(10-methoxy-3-oxo-2,9-diazatricyclo[6.3.1.04, 12]dodeca-1(11),4,6,8(12),9-pentaen-2-yl)piperidine-2,6-dione (20 mg, 64.25 μmol) in acetonitrile (1 mL), were added chloro(trimethyl)silane (13.96 mg, 128.50 μmol, 16.31 μL) and sodium iodide (19.26 mg, 128.50 mol, 5.25 μL). The resulting mixture was heated at 70° C. for 1 h. The reaction mixture was concentrated under reduced pressure to give the residue. The residue was treated with saturated sodium thiosulfate solution, the precipitated solid was filtered, washed with water (5 mL) and dried under vacuum to give 2-(2,6-dioxo-3-piperidyl)-2,9-diazatricyclo[6.3.1.04, 12]dodeca-1(11),4,6,8(12)-tetraene-3,10-dione Compound 223 (16.0 mg, 52.77 μmol, 82.13% yield) as an off-white solid. LCMS (ES⁺): m/z 298.0 [M+H]⁺. ¹H NMR (400 MHz, DMSO-d₆): δ 11.55 (s, 1H), 11.15 (s, 1H), 7.74 (t, J=8.0 Hz, 1H), 7.61 (d, J=6.8 Hz, 1H), 7.43 (d, J=8.0 Hz, 1H), 6.27 (d, J=1.2 Hz, 1H), 5.37-5.32 (m, 1H), 2.91-2.84 (m, 1H), 2.75-2.65 (m, 2H), 2.10-2.07 (m, 1H) ppm.

### Example 104: NanoBRET™ Assay

The cell permeability and binding affinity of test compounds to cellular cereblon (CRBN) was determined by competitive displacement of a pomalidomide-NanoBRET™ tracer reversibly bound to a CRBN-NanoLuc® fusion protein in 293T cells. 293T cells were modified by lentiviral transfection to express a fusion of CRBN and NanoLuc® luciferase. The modified CRBN-NanoLuc 293T cell line (named 293T.116) was co-treated with varying concentrations of test compound and a pomalidomide probe conjugated with NanoBRET fluorescent tracer at its predetermined KD concentration (300 nM) and incubated for 2 hr at 37° C. to reach equilibrium. Affinity of test compound was determined by displacement of NanoBRET-pomalidomide tracer signal following the addition of NanoBRET reagents (Promega) per manufacturer's instructions.

40 μL 293T.116 cells suspended in OptiMEM media at 2×105 cells/mL (8000 cells/well) were dispensed using a Multidrop Combi Reagent Dispenser (Thermo Fisher) to each well of 384-well white TC-treated microplates. 10 mM DMSO test compound stock solution was serially diluted (half log) in DMSO to generate 11-point dose series (10000, 3160, 1000, 316, 100, 31.6, 10, 3.2, 1, 0.3, 0.1 μM) in an acoustic ready 384-well low dead volume microplate (Labcyte). Using Echo 550 Acoustic Liquid Handler (Labcyte), 40 nL of serially diluted compound solutions were dispensed in duplicate to each 384-well white TC-treated microplate containing 293T.116 cells. 40 nL DMSO was transferred to all control wells. 40 nL NanoBRET-pomalidomide tracer was dispensed to all wells in column 1-23. 40 nL additional DMSO was dispensed to column 24. Final concentration of DMSO was 0.2% for all samples. Plates were spun briefly and cells were incubated at 37° C.; 5% CO₂ for 2 hr. 20 μL NanoBRET TE Assay reagents were added to each well and NanoBRET signal was acquired on an EnVision Multilabel Reader (PerkinElmer). Donor emission from CRBN-NanoLuc was detected at 450 nm with a NanoLuc 460/50 filter and Acceptor fluorescence of NanoBRET-pomalidomide tracer (618 nm) was detected with a 600 nm long pass NanoBRET filter. Ratio of Acceptor signal/Donor signal was calculated for each well. Column 24 (cells without NanoBRET-pomalidomide tracer addition) was used as positive control (P).

Percent response of compound-treated samples (T) were calculated by normalizing the Acceptor/Donor ratio for each well to the DMSO treated negative (N) controls on the same microtiter plate after background (i.e. positive control) signal subtraction: Response %=100×(Signal(T)−Average (P))/(Average (N)−Average (P))

### Example 105: CRBN FP Binding Assay

The determination of the binding constant (KD) of test compounds to CRBN-DDB1 was carried out using an established responsive and quantitative in vitro fluorescence polarization (FP) binding assay. Control compounds were run on the same plate. Compounds were dispensed from serially diluted DMSO stock supplied by Frontier Scientific Services Inc in low dead volume plates into black 384-well compatible FP plates using acoustic technology to 1% of total reaction volume. Compounds were arranged vertically in rows A through P. Concentrations series are horizontal: columns 1-11, and then duplicates in columns 12-22. Columns 23 and 24 are reserved for 0% (5 nM probe) and 100% controls (protein at high concentration with 5 nM probe), respectively. Compound binding to CRBN-DDB1 was measured by displacement of Alexa-647 Fluor® based probe with a KD of 113 nM, as determined by a single site ligand depletion model. A 20 μL mixture containing 150 nM CRBN-DDB1 and 5 nM probe dye in 50 mM HEPES, pH 7.4, 200 mM NaCl, 1 mM TCEP and 0.05% pluronic acid-127 was added to wells containing compound and incubated at room temperature for 1.5 hours. Controls wells with 100% bound probe contained 1500 nM of CRBN. Matching control plates excluding CRBN-DDB1 were used to correct for background fluorescence. Plates were read on an Envision plate reader with appropriate FP filter sets.

| | | NanoBRET |
|---|---|---|
| | | 293T.116 |
| Compound | | 2 h IC50 |
| # | Structure | (nM) |
| 224 | 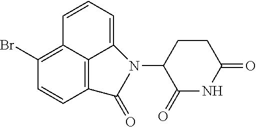 [SMILES: O=C1CCC(N2C(=O)c3ccc(Br)c4cccc2c34)C(=O)N1] | 40 |
| 225 | 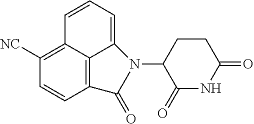 [SMILES: N#Cc1ccc2c3c(cccc13)N(C1CCC(=O)NC1=O)C2=O] | 60 |

### Example 106: GSPT1 Degradation Assay

### HiBit Method GSPT1

### Materials

DMEM no-phenol red medium and fetal bovine serum (FBS) were purchased from Gibco (Grand Island, NY, USA). Nano-Glo® HiBiT Lytic Assay System was purchased from Promega (Madison, WI, USA). 293T.114 (HiBiT-GSPT1) cell line was generated in house, endogenously expressing GSPT1 with HiBiT fusion tag via CRISPR. Cell culture flasks and 384-well microplates were acquired from VWR (Radnor, PA, USA).

### GSPT1 Degradation Analysis

GSPT1 degradation was determined based on quantification of luminescent signal using Nano-Glo® HiBiT Lytic Assay kit. Test compounds were added to the 384-well plate from a top concentration of 10 μM with 5 points, 3-fold titration in quadruplicates. 293T.114 cells were added into 384-well plates at a cell density of 6000 cells per well. The plates were kept at 37° C. with 5% CO₂ for 24 hours. The cells treated in the absence of the test compound were the negative control and the cells without Nano-Glo® HiBiT Lytic reagent were the positive control. After 24-hour incubation, Nano-Glo® HiBiT Lytic Assay reagents were added to the designated wells. Luminescence was acquired on EnVision™ Multilabel Reader (PerkinElmer, Santa Clara, CA, USA).

### HiBit Method SALL4

### Materials

RPMI 1640 Medium without phenol red, fetal bovine serum (FBS), and Sodium Pyruvate (100 mM) were purchased from Gibco (Grand Island, NY, USA). Nano-Glo® HiBiT Lytic Assay System was purchased from Promega (Madison, WI, USA). KELLY.2 (SALL4-HiBiT) cell line, endogenously expressing SALL4 with HiBiT fusion tag via CRISPR, was made internally. Cell culture flasks and 384-well microplates were acquired from VWR (Radnor, PA, USA).

### SALL4 Degradation Analysis

SALL4 degradation was determined based on quantification of luminescent signal using Nano-Glo® HiBiT Lytic Assay kit. Test compounds were added to the 384-well plate from a top concentration of 10 μM with 11 points, half log titration in duplicates. KELLY.2 cells were added into 384-well plates at a cell density of 6000 cells per well. The plates were kept at 37° C. with 5% CO₂ for 6 hours. The cells treated in the absence of the test compound were the negative control and the cells without Nano-Glo® HiBiT Lytic reagent were the positive control. After 6-hour incubation, Nano-Glo® HiBiT Lytic Assay reagents were added to the cells. Luminescence was acquired on EnVision™ Multilabel Reader (PerkinElmer, Santa Clara, CA, USA).

### HiBiT Method IKZF1

### Materials

RPMI no-phenol red medium and fetal bovine serum (FBS) were purchased from Gibco (Grand Island, NY, USA). Nano-Glo® HiBiT Lytic Assay System was purchased from Promega (Madison, WI, USA). NCIH929.11 (HiBiT-IKZF1) cell line was generated in house, endogenously expressing IKZF1 with HiBiT fusion tag via CRISPR. Cell culture flasks and 384-well microplates were acquired from VWR (Radnor, PA, USA).

### IKZF1 Degradation Analysis

IKZF 1 degradation was determined based on quantification of luminescent signal using Nano-Glo® HiBiT Lytic Assay kit. Test compounds were added to the 384-well plate from a top concentration of 10 μM with 11 points, half-log titration in duplicates. NCIH929.11 cells expressing HiBiT-tagged TKZF1 were added into 384-well plates in RPMI medium containing 10% FBS and 0.05 mM 2-mercaptoethanol at a cell density of 15000 cells per well. The plates were kept at 37° C. with 500 CO₂ for 6 hours. Cells treated in the absence of the test compound were the negative control and wells containing media only were the positive control. After 6-hour incubation, Nano-Glo® HiBiT Lytic Assay reagents were added to the designated wells. Luminescence was acquired on EnVision™ Multilabel Reader (PerkinElmer, Santa Clara, CA, USA).

TABLE 1
GSPT1 degradation of selected compounds

| Compound | | GSPT1 Eₘₐₓ @ |
|---|---|---|
| # | Structure | 10 μM 24 Hours |
| 60 | 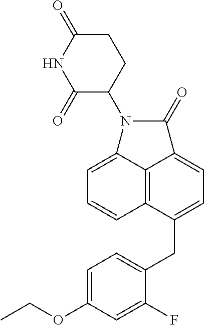 [SMILES: CCOc1ccc(Cc2ccc3c4c(cccc24)N(C2CCC(=O)NC2=O)C3=O)c(F)c1] | ++++ |
| 61 | 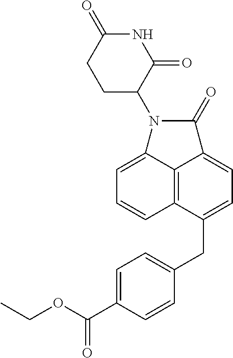 [SMILES: CCOC(=O)c1ccc(Cc2ccc3c4c(cccc24)N(C2CCC(=O)NC2=O)C3=O)cc1] | ++++ |
| 62 | 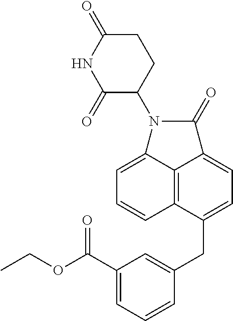 [SMILES: CCOC(=O)c1cccc(Cc2ccc3c4c(cccc24)N(C2CCC(=O)NC2=O)C3=O)c1] | ++++ |
| 63 | 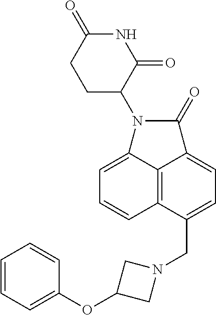 [SMILES: O=C1CCC(N2C(=O)c3ccc(CN4CC(Oc5ccccc5)C4)c4cccc2c34)C(=O)N1] | ++++ |
| 64 | 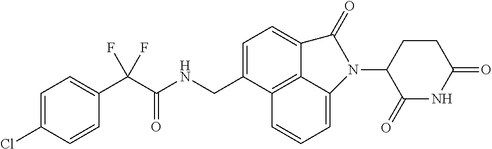 [SMILES: O=C1CCC(N2C(=O)c3ccc(CNC(=O)C(F)(F)c4ccc(Cl)cc4)c4cccc2c34)C(=O)N1] | ++++ |

As used in the table above for Emax values <45% = ++++, 45-60% = +++, 61-95% = ++, >95% = +

TABLE 2
IKZF1, SALL4, and GSPT1 Degradation Data

| | HiBiT- | | HiBiT- | | HiBiT- | |
|---|---|---|---|---|---|---|
| | Degradation | | Degradation | | Degradation | |
| | NCHI929.11 | | KELLY.2 | | 293T.114 | |
| | IKZF1 | | SALL4 | | GSPT1 | |
| | 6 hours | 6 | 6 hours | | 6 hours | |
| | DC₅₀ | hours | DC₅₀ | 6 hours | DC₅₀ | 6 hours |
| Structure | nM | Emax | nM | Emax | nM | Emax |
| 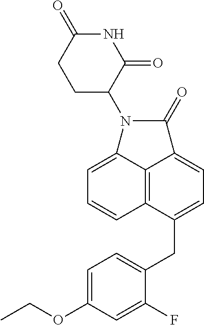 [SMILES: CCOc1ccc(Cc2ccc3c4c(cccc24)N(C2CCC(=O)NC2=O)C3=O)c(F)c1] | + | ++ | + | ++ | + | ++ |
| Compound 60 | | | | | | |
| 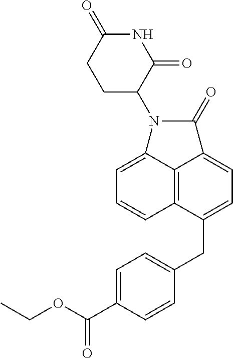 [SMILES: CCOC(=O)c1ccc(Cc2ccc3c4c(cccc24)N(C2CCC(=O)NC2=O)C3=O)cc1] | +++ | ++++ | +++ | ++++ | + | +++ |
| Compound 61 | | | | | | |
| 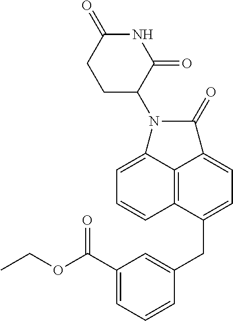 [SMILES: CCOC(=O)c1cccc(Cc2ccc3c4c(cccc24)N(C2CCC(=O)NC2=O)C3=O)c1] | + | ++ | + | ++ | + | ++ |
| Compound 62 | | | | | | |
| 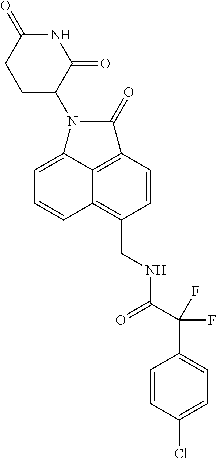 [SMILES: O=C1CCC(N2C(=O)c3ccc(CNC(=O)C(F)(F)c4ccc(Cl)cc4)c4cccc2c34)C(=O)N1] | + | +++ | ++++ | ++++ | + | +++ |
| Compound 64 | | | | | | |
| 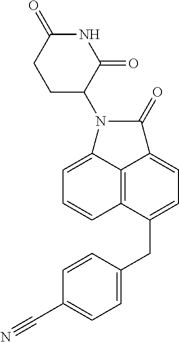 [SMILES: N#Cc1ccc(Cc2ccc3c4c(cccc24)N(C2CCC(=O)NC2=O)C3=O)cc1] | + | ++ | +++ | +++ | | |
| Compound 66 | | | | | | |
| 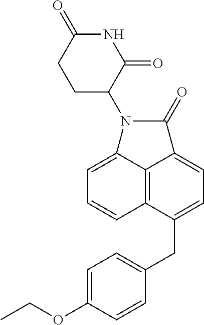 [SMILES: CCOc1ccc(Cc2ccc3c4c(cccc24)N(C2CCC(=O)NC2=O)C3=O)cc1] | + | ++ | + | ++ | + | ++ |
| Compound 68 | | | | | | |
| 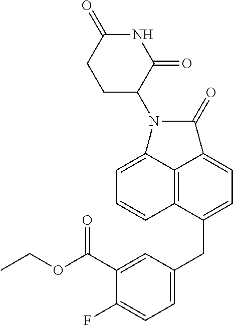 [SMILES: CCOC(=O)c1cc(Cc2ccc3c4c(cccc24)N(C2CCC(=O)NC2=O)C3=O)ccc1F] | + | ++ | + | ++ | + | ++ |
| Compound 69 | | | | | | |
| 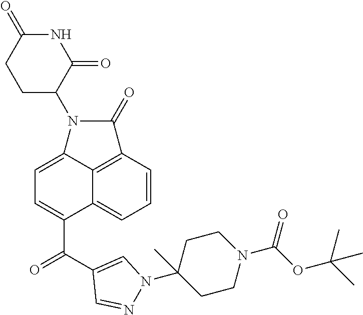 [SMILES: CC(C)(C)OC(=O)N1CCC(C)(n2cc(C(=O)c3ccc4c5c(cccc35)C(=O)N4C3CCC(=O)NC3=O)cn2)CC1] | ++++ | ++++ | ++++ | ++++ | + | ++ |
| Compound 70 | | | | | | |
| 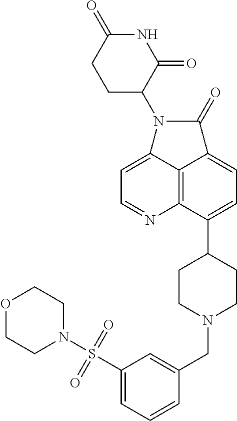 [SMILES: O=C1CCC(N2C(=O)c3ccc(C4CCN(Cc5cccc(S(=O)(=O)N6CCOCC6)c5)CC4)c4nccc2c34)C(=O)N1] | | | ++++ | ++++ | >100 | ++ |
| Compound 77 | | | | | | |
| 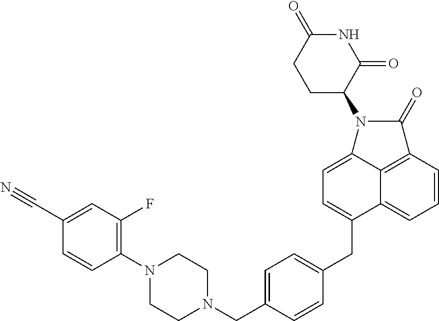 [SMILES: N#Cc1ccc(N2CCN(Cc3ccc(Cc4ccc5c6c(cccc46)C(=O)N5[C@H]4CCC(=O)NC4=O)cc3)CC2)c(F)c1] | ++++ | ++++ | ++++ | ++++ | + | ++ |
| Compound 78 | | | | | | |
| 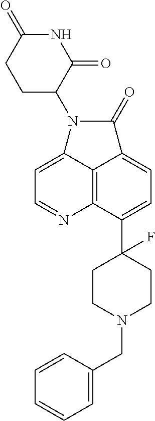 [SMILES: O=C1CCC(N2C(=O)c3ccc(C4(F)CCN(Cc5ccccc5)CC4)c4nccc2c34)C(=O)N1] | | | ++++ | ++++ | >100 | ++ |
| Compound 81 | | | | | | |
| 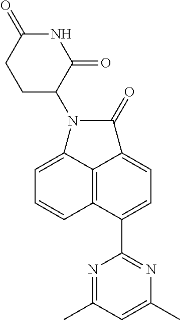 [SMILES: Cc1cc(C)nc(-c2ccc3c4c(cccc24)N(C2CCC(=O)NC2=O)C3=O)n1] | | | ++++ | ++++ | >100 | ++ |
| Compound 87 | | | | | | |
| 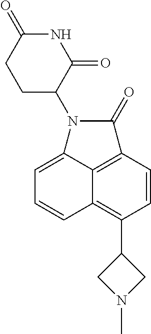 [SMILES: CN1CC(c2ccc3c4c(cccc24)N(C2CCC(=O)NC2=O)C3=O)C1] | | | ++++ | ++++ | >100 | ++ |
| Compound 90 | | | | | | |
| 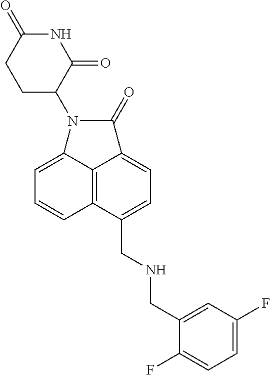 [SMILES: O=C1CCC(N2C(=O)c3ccc(CNCc4cc(F)ccc4F)c4cccc2c34)C(=O)N1] | + | +++ | ++++ | ++++ | | |
| Compound 155 | | | | | | |
| 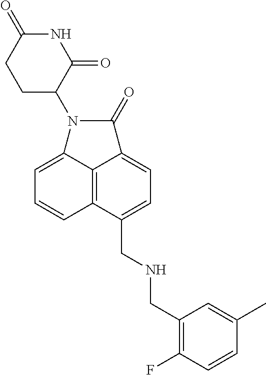 [SMILES: Cc1ccc(F)c(CNCc2ccc3c4c(cccc24)N(C2CCC(=O)NC2=O)C3=O)c1] | ++ | +++ | ++++ | ++++ | + | ++ |
| Compound 156 | | | | | | |
| 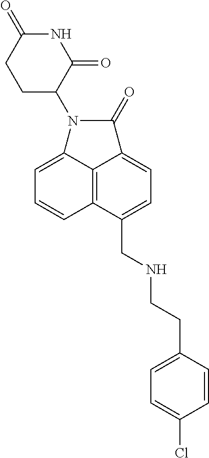 [SMILES: O=C1CCC(N2C(=O)c3ccc(CNCCc4ccc(Cl)cc4)c4cccc2c34)C(=O)N1] | +++ | ++++ | ++++ | ++++ | | |
| Compound 157 | | | | | | |
| 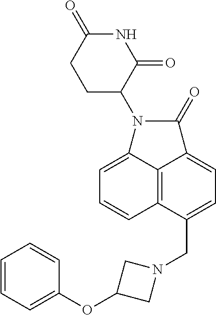 [SMILES: O=C1CCC(N2C(=O)c3ccc(CN4CC(Oc5ccccc5)C4)c4cccc2c34)C(=O)N1] | ++++ | ++++ | ++++ | ++++ | ++++ | ++++ |
| Compound 158 | | | | | | |
| 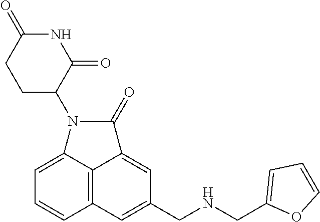 [SMILES: O=C1CCC(N2C(=O)c3cc(CNCc4ccco4)cc4cccc2c34)C(=O)N1] | | | | | + | +++ |
| Compound 160 | | | | | | |
| 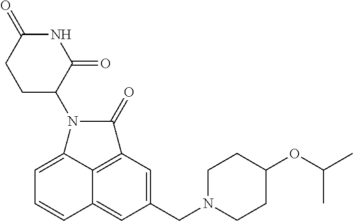 [SMILES: CC(C)OC1CCN(Cc2cc3c4c(cccc4c2)N(C2CCC(=O)NC2=O)C3=O)CC1] | ++ | ++++ | + | ++ | | |
| Compound 161 | | | | | | |
| 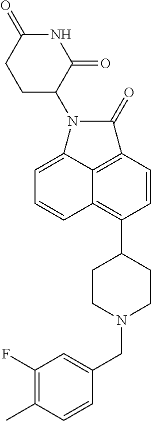 [SMILES: Cc1ccc(CN2CCC(c3ccc4c5c(cccc35)N(C3CCC(=O)NC3=O)C4=O)CC2)cc1F] | | | + | ++ | + | ++ |
| Compound 176 | | | | | | |
| 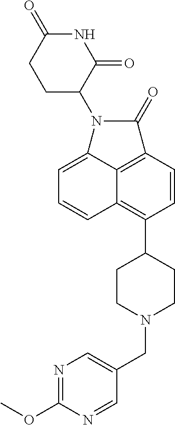 [SMILES: COc1ncc(CN2CCC(c3ccc4c5c(cccc35)N(C3CCC(=O)NC3=O)C4=O)CC2)cn1] | | | + | ++ | + | ++ |
| Compound 177 | | | | | | |
| 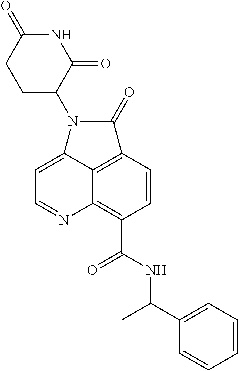 [SMILES: CC(NC(=O)c1ccc2c3c(ccnc13)N(C1CCC(=O)NC1=O)C2=O)c1ccccc1] | | | ++++ | ++++ | >100 | ++ |
| Compound 185 | | | | | | |
| 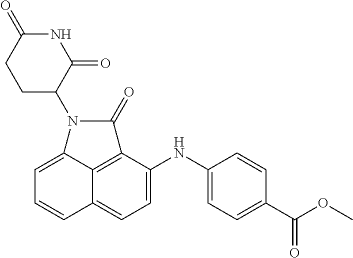 [SMILES: COC(=O)c1ccc(Nc2ccc3cccc4c3c2C(=O)N4C2CCC(=O)NC2=O)cc1] | + | ++ | + | ++ | + | ++ |
| Compound 186 | | | | | | |
| 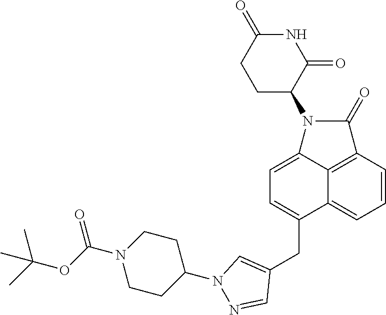 [SMILES: CC(C)(C)OC(=O)N1CCC(n2cc(Cc3ccc4c5c(cccc35)C(=O)N4[C@H]3CCC(=O)NC3=O)cn2)CC1] | ++++ | ++++ | ++++ | ++++ | + | ++ |
| Compound 197 | | | | | | |
| 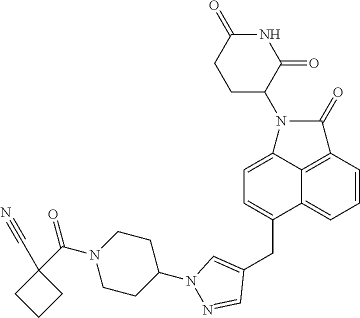 [SMILES: N#CC1(C(=O)N2CCC(n3cc(Cc4ccc5c6c(cccc46)C(=O)N5C4CCC(=O)NC4=O)cn3)CC2)CCC1] | | | ++++ | ++++ | + | + |
| Compound 200 | | | | | | |
| 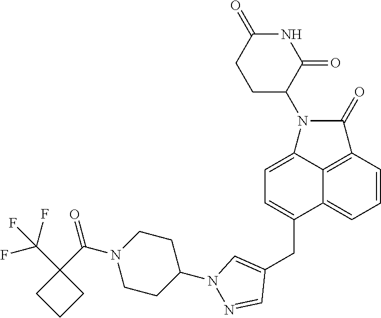 [SMILES: O=C1CCC(N2C(=O)c3cccc4c(Cc5cnn(C6CCN(C(=O)C7(C(F)(F)F)CCC7)CC6)c5)ccc2c34)C(=O)N1] | | | ++++ | ++++ | + | + |
| Compound 201 | | | | | | |
| 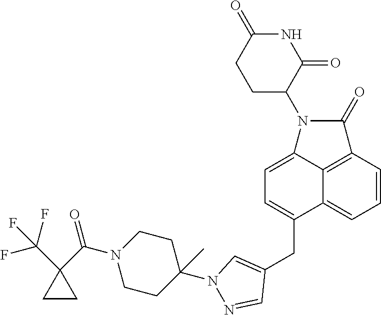 [SMILES: CC1(n2cc(Cc3ccc4c5c(cccc35)C(=O)N4C3CCC(=O)NC3=O)cn2)CCN(C(=O)C2(C(F)(F)F)CC2)CC1] | | | ++++ | ++++ | + | ++ |
| Compound 203 | | | | | | |
| 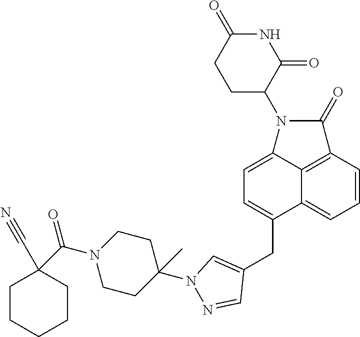 [SMILES: CC1(n2cc(Cc3ccc4c5c(cccc35)C(=O)N4C3CCC(=O)NC3=O)cn2)CCN(C(=O)C2(C#N)CCCCC2)CC1] | | | ++++ | ++++ | + | ++ |
| Compound 204 | | | | | | |
| 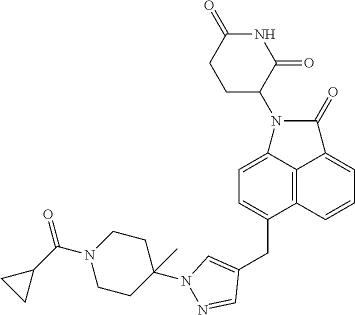 [SMILES: CC1(n2cc(Cc3ccc4c5c(cccc35)C(=O)N4C3CCC(=O)NC3=O)cn2)CCN(C(=O)C2CC2)CC1] | | | ++++ | ++++ | + | ++ |
| Compound 205 | | | | | | |
| 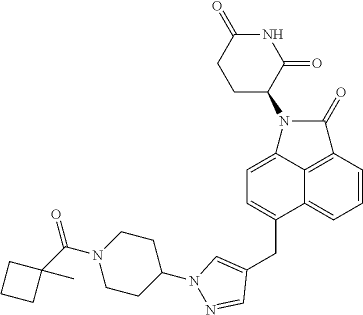 [SMILES: CC1(C(=O)N2CCC(n3cc(Cc4ccc5c6c(cccc46)C(=O)N5[C@H]4CCC(=O)NC4=O)cn3)CC2)CCC1] | ++++ | ++++ | ++++ | ++++ | + | + |
| Compound 206 | | | | | | |
| 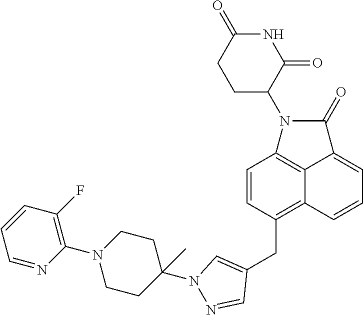 [SMILES: CC1(n2cc(Cc3ccc4c5c(cccc35)C(=O)N4C3CCC(=O)NC3=O)cn2)CCN(c2ncccc2F)CC1] | | | ++++ | ++++ | + | ++ |
| Compound 208 | | | | | | |
| 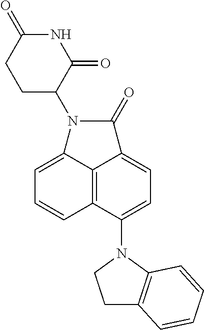 [SMILES: O=C1CCC(N2C(=O)c3ccc(N4CCc5ccccc54)c4cccc2c34)C(=O)N1] | + | ++ | ++++ | ++++ | + | ++ |
| Compound 209 | | | | | | |
| 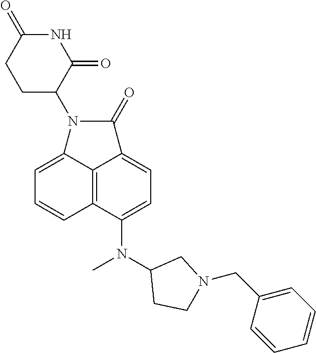 [SMILES: CN(c1ccc2c3c(cccc13)N(C1CCC(=O)NC1=O)C2=O)C1CCN(Cc2ccccc2)C1] | | | | | >100 | ++ |
| Compound 220 | | | | | | |
| 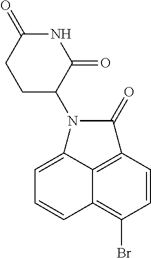 [SMILES: O=C1CCC(N2C(=O)c3ccc(Br)c4cccc2c34)C(=O)N1] | + | ++ | ++++ | ++++ | + | ++ |
| Compound 224 | | | | | | |

As used in the table above for DC₅₀ values <100 nM = ++++, 100-1,000 nM = +++, 1,001-10,000 nM = ++, >10,000 nM = +
For Emax values <45% = ++++, 45-60% = +++, 60-95% = ++, >95% = +

TABLE 3
GSPT1 Degradation and CRBN Binding

| | HiBiT-Degradation | | | |
|---|---|---|---|---|
| | 293T.114 | | | |
| | GSPT1 | | | |
| | 24 hours | 24 hours | 48 hours | 48 hours |
| Structure | DC₅₀ nM | Emax | DC₅₀ nM | Emax |
| 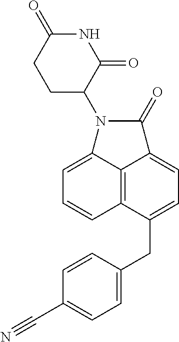 [SMILES: N#Cc1ccc(Cc2ccc3c4c(cccc24)N(C2CCC(=O)NC2=O)C3=O)cc1] | + | ++ | + | ++ |
| Compound 66 | | | | |
| 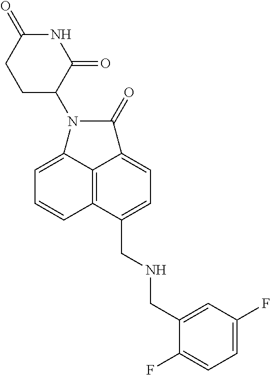 [SMILES: O=C1CCC(N2C(=O)c3ccc(CNCc4cc(F)ccc4F)c4cccc2c34)C(=O)N1] | + | ++ | + | ++ |
| Compound 155 | | | | |
| 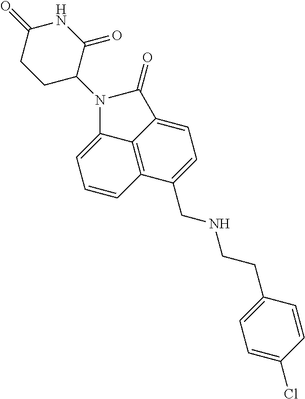 [SMILES: O=C1CCC(N2C(=O)c3ccc(CNCCc4ccc(Cl)cc4)c4cccc2c34)C(=O)N1] | + | ++ | + | ++ |
| Compound 157 | | | | |
| 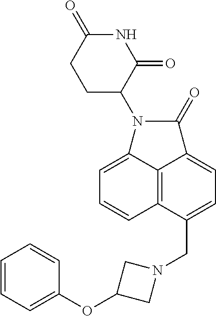 [SMILES: O=C1CCC(N2C(=O)c3ccc(CN4CC(Oc5ccccc5)C4)c4cccc2c34)C(=O)N1] | ++++ | ++++ | | |
| Compound 158 | | | | |
| 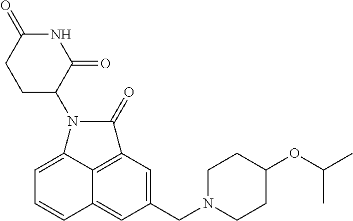 [SMILES: CC(C)OC1CCN(Cc2cc3c4c(cccc4c2)N(C2CCC(=O)NC2=O)C3=O)CC1] | + | ++ | + | ++ |
| Compound 161 | | | | |

As used in the table above for DC₅₀ values <100 nM = ++++, 100-1,000 nM = +++, 1,001-10,000 nM = ++, >10,000 nM = +
For Emax values <45% = ++++, 45-60% = +++, 60-95% = ++, >95% = +

TABLE 4
CRBN Binding

| | NanoBRET |
|---|---|
| | 293T.116 |
| | CRBN |
| | 2 hours |
| Structure | IC₅₀ nM |
| 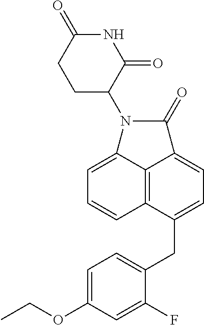 [SMILES: CCOc1ccc(Cc2ccc3c4c(cccc24)N(C2CCC(=O)NC2=O)C3=O)c(F)c1] | ++++ |
| Compound 60 | |
| 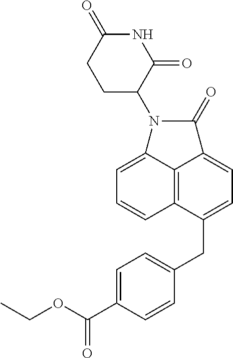 [SMILES: CCOC(=O)c1ccc(Cc2ccc3c4c(cccc24)N(C2CCC(=O)NC2=O)C3=O)cc1] | ++++ |
| Compound 61 | |
| 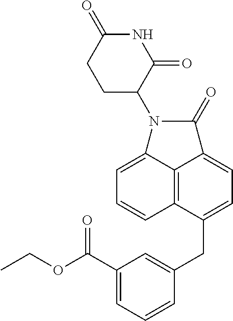 [SMILES: CCOC(=O)c1cccc(Cc2ccc3c4c(cccc24)N(C2CCC(=O)NC2=O)C3=O)c1] | ++++ |
| Compound 62 | |
| 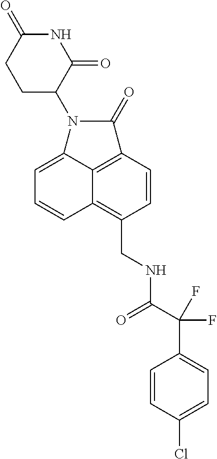 [SMILES: O=C1CCC(N2C(=O)c3ccc(CNC(=O)C(F)(F)c4ccc(Cl)cc4)c4cccc2c34)C(=O)N1] | ++++ |
| Compound 64 | |
| 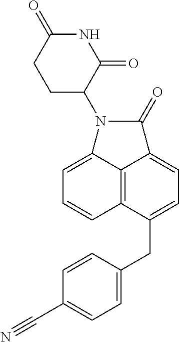 [SMILES: N#Cc1ccc(Cc2ccc3c4c(cccc24)N(C2CCC(=O)NC2=O)C3=O)cc1] | ++++ |
| Compound 66 | |
| 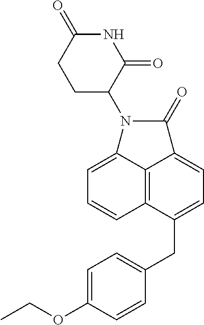 [SMILES: CCOc1ccc(Cc2ccc3c4c(cccc24)N(C2CCC(=O)NC2=O)C3=O)cc1] | ++++ |
| Compound 68 | |
| 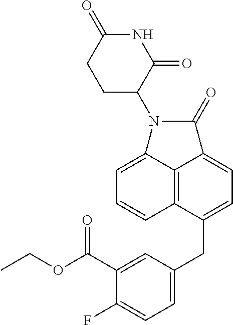 [SMILES: CCOC(=O)c1cc(Cc2ccc3c4c(cccc24)N(C2CCC(=O)NC2=O)C3=O)ccc1F] | ++++ |
| Compound 69 | |
| 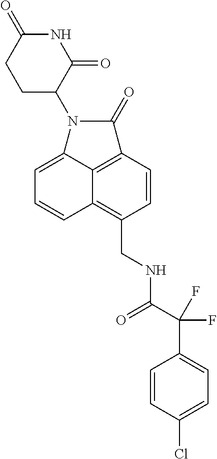 [SMILES: O=C1CCC(N2C(=O)c3ccc(CNC(=O)C(F)(F)c4ccc(Cl)cc4)c4cccc2c34)C(=O)N1] | +++ |
| Compound 70 | |
| 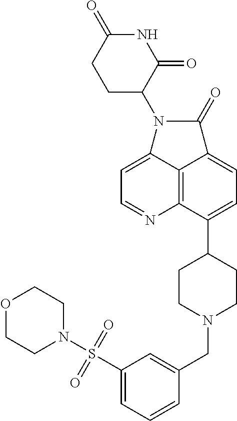 [SMILES: O=C1CCC(N2C(=O)c3ccc(C4CCN(Cc5cccc(S(=O)(=O)N6CCOCC6)c5)CC4)c4nccc2c34)C(=O)N1] | |
| Compound 77 | |
| 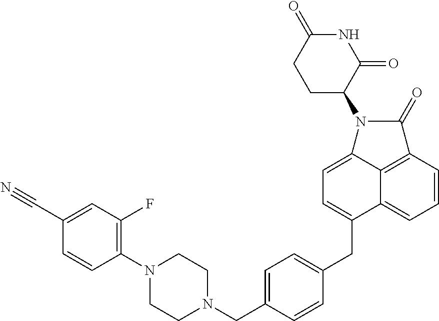 [SMILES: N#Cc1ccc(N2CCN(Cc3ccc(Cc4ccc5c6c(cccc46)C(=O)N5[C@H]4CCC(=O)NC4=O)cc3)CC2)c(F)c1] | ++++ |
| Compound 78 | |
| 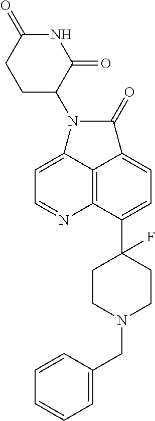 [SMILES: O=C1CCC(N2C(=O)c3ccc(C4(F)CCN(Cc5ccccc5)CC4)c4nccc2c34)C(=O)N1] | |
| Compound 81 | |
| 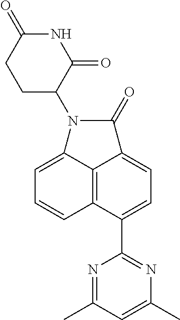 [SMILES: Cc1cc(C)nc(-c2ccc3c4c(cccc24)N(C2CCC(=O)NC2=O)C3=O)n1] | |
| Compound 87 | |
| 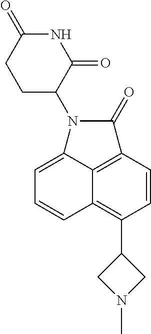 [SMILES: CN1CC(c2ccc3c4c(cccc24)N(C2CCC(=O)NC2=O)C3=O)C1] | |
| Compound 90 | |
| 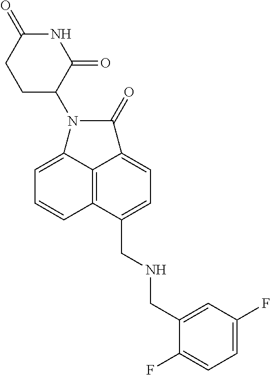 [SMILES: O=C1CCC(N2C(=O)c3ccc(CNCc4cc(F)ccc4F)c4cccc2c34)C(=O)N1] | ++++ |
| Compound 155 | |
| 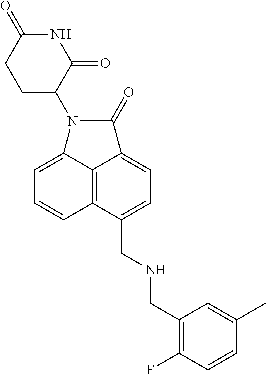 [SMILES: Cc1ccc(F)c(CNCc2ccc3c4c(cccc24)N(C2CCC(=O)NC2=O)C3=O)c1] | ++++ |
| Compound 156 | |
| 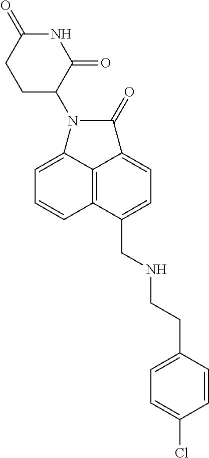 [SMILES: O=C1CCC(N2C(=O)c3ccc(CNCCc4ccc(Cl)cc4)c4cccc2c34)C(=O)N1] | ++++ |
| Compound 157 | |
| 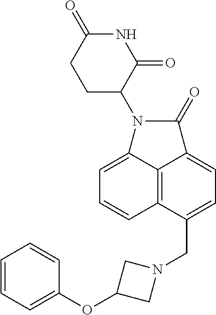 [SMILES: O=C1CCC(N2C(=O)c3ccc(CN4CC(Oc5ccccc5)C4)c4cccc2c34)C(=O)N1] | ++++ |
| Compound 158 | |
| 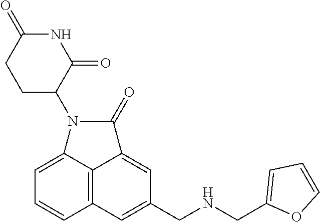 [SMILES: O=C1CCC(N2C(=O)c3cc(CNCc4ccco4)cc4cccc2c34)C(=O)N1] | |
| Compound 160 | |
| 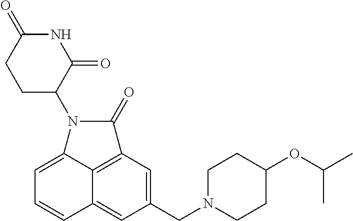 [SMILES: CC(C)OC1CCN(Cc2cc3c4c(cccc4c2)N(C2CCC(=O)NC2=O)C3=O)CC1] | ++++ |
| Compound 161 | |
| 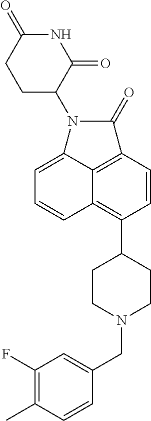 [SMILES: Cc1ccc(CN2CCC(c3ccc4c5c(cccc35)N(C3CCC(=O)NC3=O)C4=O)CC2)cc1F] | ++++ |
| Compound 176 | |
| 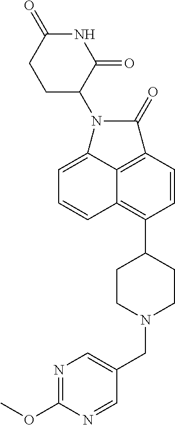 [SMILES: COc1ncc(CN2CCC(c3ccc4c5c(cccc35)N(C3CCC(=O)NC3=O)C4=O)CC2)cn1] | ++++ |
| Compound 177 | |
| 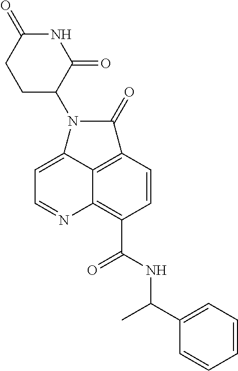 [SMILES: CC(NC(=O)c1ccc2c3c(ccnc13)N(C1CCC(=O)NC1=O)C2=O)c1ccccc1] | |
| Compound 185 | |
| 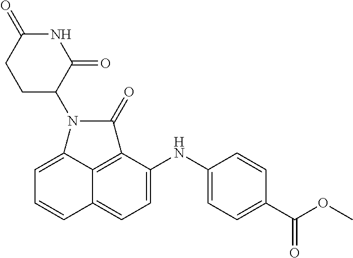 [SMILES: COC(=O)c1ccc(Nc2ccc3cccc4c3c2C(=O)N4C2CCC(=O)NC2=O)cc1] | |
| Compound 186 | |
| 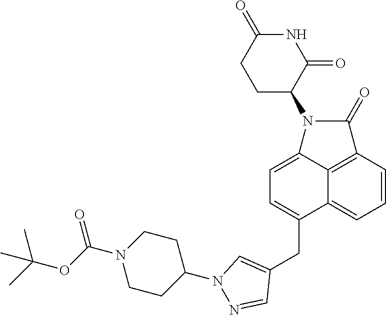 [SMILES: CC(C)(C)OC(=O)N1CCC(n2cc(Cc3ccc4c5c(cccc35)C(=O)N4[C@H]3CCC(=O)NC3=O)cn2)CC1] | |
| Compound 197 | |
| 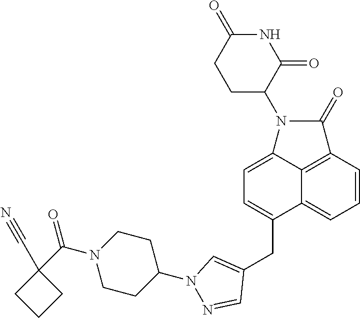 [SMILES: N#CC1(C(=O)N2CCC(n3cc(Cc4ccc5c6c(cccc46)C(=O)N5C4CCC(=O)NC4=O)cn3)CC2)CCC1] | |
| Compound 200 | |
| 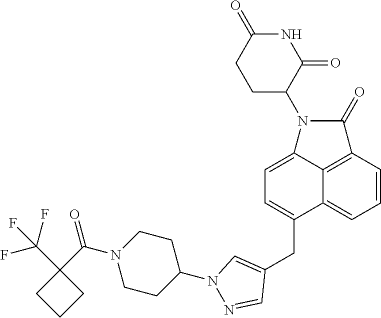 [SMILES: O=C1CCC(N2C(=O)c3cccc4c(Cc5cnn(C6CCN(C(=O)C7(C(F)(F)F)CCC7)CC6)c5)ccc2c34)C(=O)N1] | ++++ |
| Compound 201 | |
| 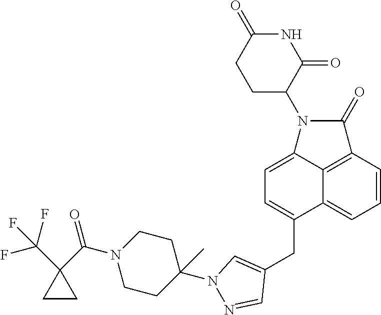 [SMILES: CC1(n2cc(Cc3ccc4c5c(cccc35)C(=O)N4C3CCC(=O)NC3=O)cn2)CCN(C(=O)C2(C(F)(F)F)CC2)CC1] | ++++ |
| Compound 203 | |
| 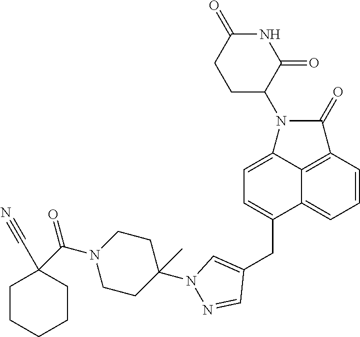 [SMILES: CC1(n2cc(Cc3ccc4c5c(cccc35)C(=O)N4C3CCC(=O)NC3=O)cn2)CCN(C(=O)C2(C#N)CCCCC2)CC1] | ++++ |
| Compound 204 | |
| 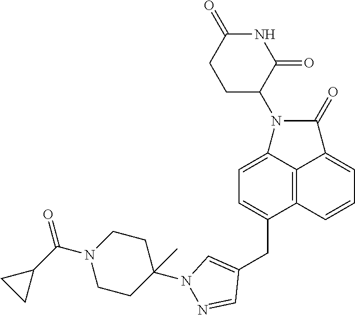 [SMILES: CC1(n2cc(Cc3ccc4c5c(cccc35)C(=O)N4C3CCC(=O)NC3=O)cn2)CCN(C(=O)C2CC2)CC1] | ++++ |
| Compound 205 | |
| 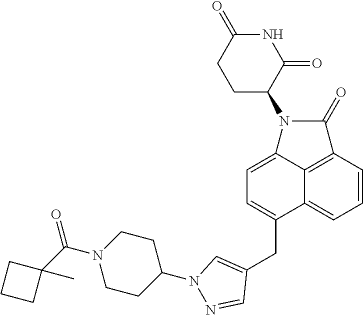 [SMILES: CC1(C(=O)N2CCC(n3cc(Cc4ccc5c6c(cccc46)C(=O)N5[C@H]4CCC(=O)NC4=O)cn3)CC2)CCC1] | ++++ |
| Compound 206 | |
| 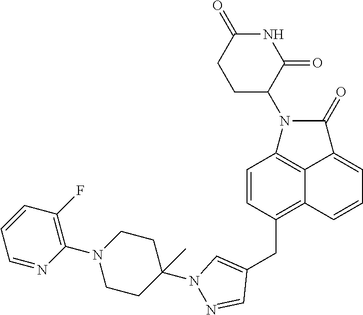 [SMILES: CC1(n2cc(Cc3ccc4c5c(cccc35)C(=O)N4C3CCC(=O)NC3=O)cn2)CCN(c2ncccc2F)CC1] | |
| Compound 208 | |
| 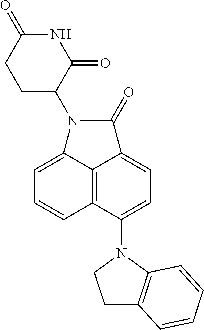 [SMILES: O=C1CCC(N2C(=O)c3ccc(N4CCc5ccccc54)c4cccc2c34)C(=O)N1] | |
| Compound 209 | |
| 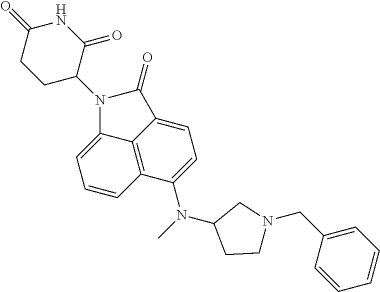 [SMILES: CN(c1ccc2c3c(cccc13)N(C1CCC(=O)NC1=O)C2=O)C1CCN(Cc2ccccc2)C1] | |
| Compound 220 | |
| 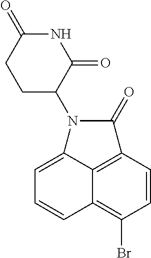 [SMILES: O=C1CCC(N2C(=O)c3ccc(Br)c4cccc2c34)C(=O)N1] | ++++ |
| Compound 224 | |

As used in the table above for IC₅₀ values <100 nM = ++++, 100-1,000 nM = +++, 1,001-10,000 nM = ++, >10,000 nM = +
For Emax values <45% = ++++, 45-60% = +++, 61-95% = ++, >95% = +

All publications and patent applications cited in this specification are herein incorporated by reference as if each individual publication or patent application were specifically and individually indicated to be incorporated by reference.

Although the foregoing invention has been described in some detail by way of illustration and example for the purposes of clarity of understanding, it will be readily apparent to one of ordinary skill in the art in light of the teaching of this invention that certain changes and modifications may be made thereto without departing form the spirt or scope of the invention as defined in the claims and embodiments.

We claim:

1. A compound of Formula: 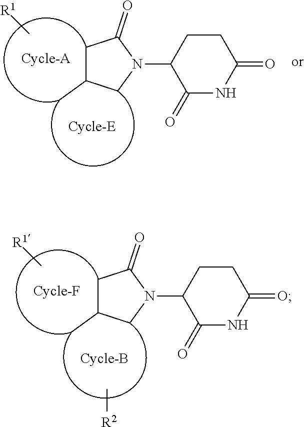
or a pharmaceutically acceptable salt thereof;
wherein
Cycle-A is a fused ring selected from the group consisting of phenyl, 5- or 6-membered heteroaryl, 5- to 8-membered heterocycle, 5- to 8-membered cycloalkyl, and 5- to 8-membered cycloalkenyl wherein Cycle-A is optionally substituted with 1, 2, or 3 substituents independently selected from R¹ as allowed by valence;
Cycle-B is a fused ring selected from the group consisting of phenyl, 5- or 6-membered heteroaryl, 5- to 8-membered heterocycle, 5- to 8-membered cycloalkyl, and 5- to 8-membered cycloalkenyl wherein Cycle-B is optionally substituted with 1, 2, or 3 substituents independently selected from R² as allowed by valence;
Cycle-E is selected from the group consisting of
(a) 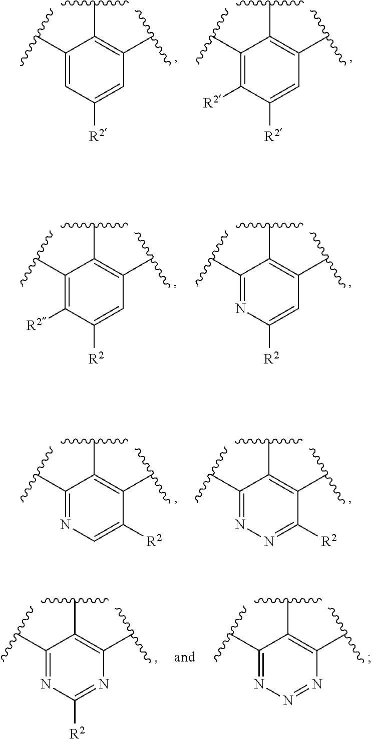
and
(b) a fused ring selected from the group consisting of 5-membered heteroaryl, 5- to 8-membered heterocycle, 5- to 8-membered cycloalkyl, or 5- to 8-membered cycloalkenyl optionally substituted with 1, 2, or 3 substituents independently selected from R² as allowed by valence;
Cycle-F is selected from the group consisting of
(a) phenyl substituted with 1, 2, or 3 substituents independently selected from R¹′; and
(b) a fused ring selected from the group consisting of 5- or 6-membered heteroaryl, 5- to 8-membered heterocycle, 5- to 8-membered cycloalkyl, or 5- to 8-membered cycloalkenyl optionally substituted with 1, 2, or 3 substituents independently selected from R¹ as allowed by valence;
R¹ and R² are each independently at each instance selected from the group consisting of
(a) hydrogen, alkyl, halogen, haloalkyl, —OR¹⁰, —SR¹⁰, —S(O)R¹², —SO₂R¹², —NR¹⁰R¹¹, cyano, nitro, heteroaryl, aryl, cycloalkyl, and heterocycle wherein each heteroaryl, aryl, cycloalkyl, and heterocycle is optionally substituted with 1, 2, 3, or 4 substituents independently selected from R⁴⁰;
(b) 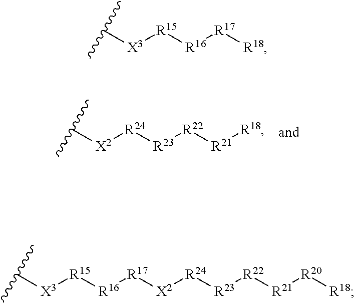
and
(c) if allowed by valence, a divalent moiety selected from the group consisting of ═O, ═S, and ═NR²⁵; and
wherein an R¹ group may optionally be combined with another R¹ group or an R² group to form a fused cycle or bicycle which may bridge Cycle-A and Cycle-B;
each X² is a bivalent moiety selected from the group consisting of bond, heterocycle, aryl, heteroaryl, bicycle, alkyl, —NR²⁷—, —CR⁴⁰R⁴¹—, —O—, —C(O)—, —C(NR²⁷)—, —C(S)—, —S(O)—, —S(O)₂—, and —S—; each of which heterocycle, aryl, heteroaryl, and bicycle is optionally substituted with 1, 2, 3, or 4 substituents independently selected from R⁴⁰;
X³ is a bivalent moiety selected from the group consisting of bond, heterocycle, aryl, heteroaryl, bicycle, —NR²⁷—, —CR⁴⁰R⁴¹—, —O—, —C(O)—, —C(NR²⁷)—, —C(S)—, —S(O)—, —S(O)₂—, —S—, arylalkyl, heterocycloalkyl, or heteroarylalkyl; each of which heterocycle, aryl, heteroaryl, and bicycle may be substituted with 1, 2, 3, or 4 substituents independently selected from R⁴⁰;
R¹′ is independently at each instance selected from the group consisting of
(a) alkyl, halogen, haloalkyl, —OR¹⁰, —SR¹⁰, —S(O)R¹², —SO₂R¹², —NR¹⁰R¹¹, cyano, nitro, heteroaryl, aryl, cycloalkyl, and heterocycle, wherein each heteroaryl, aryl and heterocycle is optionally substituted with 1, 2, 3, or 4 substituents independently selected from R⁴⁰;
(b) 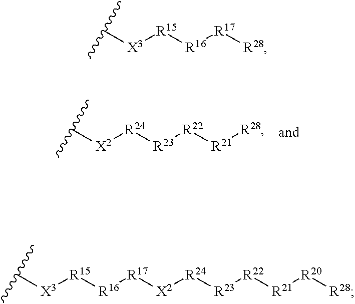
and
(c) if allowed by valence, a divalent moiety selected from the group consisting of O, S, and ═NR²⁵; and
wherein an R group may optionally be combined with another R¹′ group or an R² group to form a fused cycle or bicycle which may bridge Cycle-A and Cycle-E;
R²′ is independently at each instance selected from the group consisting of alkyl, halogen, haloalkyl, —OR¹⁰, —SR¹⁰, —S(O)R¹², —SO₂R¹², —NR¹⁰R¹¹, cyano, nitro, heteroaryl, aryl, and heterocycle; or alternatively, if allowed by valence, R²′ may be a divalent moiety selected from the group consisting of O, S, and ═NR²⁵; and wherein an R²′ group may optionally be combined with another R²′ group or an R¹ group to form a fused cycle or bicycle which may bridge Cycle-A and Cycle-E;
R²″ is independently at each instance selected from the group consisting of heteroaryl, aryl, and heterocycle, and wherein each heteroaryl, aryl and heterocycle is optionally substituted with 1, 2, 3, or 4 substituents independently selected from R⁴⁰ and wherein an R²″ group may optionally be combined with an R¹ group or an R² group to form a fused cycle or bicycle which may bridge Cycle-A and Cycle-E;
each R¹⁰ and R¹¹ are independently selected from the group consisting of hydrogen, alkyl, haloalkyl, heterocycle, aryl, heteroaryl, —C(O)R¹², —S(O)R¹², and —SO₂R¹²;
each R¹² is independently selected from the group consisting of hydrogen, alkyl, haloalkyl, heterocycle, aryl, heteroaryl, —NR¹³R¹⁴, and OR¹³;
R¹³ and R¹⁴ are each independently selected from the group consisting of hydrogen, alkyl, and haloalkyl;
R²⁸ is selected from the group consisting of alkyl, alkene, alkyne, hydroxy, azide, amino, halogen, haloalkyl, —OR¹⁰, —SR¹⁰, —S(O)R¹², —SO₂R¹², —NR¹⁰R¹¹, cyano, nitro, heteroaryl, aryl, arylalkyl, cycloalkyl, and heterocycle wherein each heteroaryl, aryl, arylalkyl, cycloalkyl, and heterocycle is optionally substituted with 1, 2, 3, or 4 substituents independently selected from R⁴⁰;
wherein if at least one of R¹⁵, R¹⁶, R¹⁷, and R²⁰ is not bond, then R²⁸ can be hydrogen;
R¹⁵, R¹⁶, and R¹⁷ are independently at each occurrence selected from the group consisting of a bond, alkyl, —C(O)—, —C(O)O—, —OC(O)—, —SO₂—, —S(O)—, —C(S)—, —C(O)NR²⁷—, —NR²⁷C(O)—, —O—, —S—, —NR²⁷—, —C(R⁴⁰R⁴¹)—, —P(O)(OR²⁶)O—, —P(O)(OR²⁶)—, bicycle, alkene, alkyne, haloalkyl, alkoxy, aryl, heterocycle, heteroaryl, lactic acid, glycolic acid arylalkyl, heterocycloalkyl and heteroarylalkyl (in either direction); each of which is optionally substituted with 1, 2, 3, or 4 substituents independently selected from R⁴⁰;
R¹⁸ is selected from the group consisting of hydrogen, alkyl, alkene, alkyne, hydroxy, azide, amino, halogen, haloalkyl, —OR¹⁰, —SR¹⁰, —S(O)R¹², —SO₂R¹², —NR¹⁰R¹¹, cyano, nitro, heteroaryl, aryl, arylalkyl, cycloalkyl, and heterocycle wherein each heteroaryl, aryl, arylalkyl, cycloalkyl, and heterocycle is optionally substituted with 1, 2, 3, or 4 substituents independently selected from R⁴⁰;
R²⁰, R²¹, R²², R²³, and R²⁴ are independently at each occurrence selected from the group consisting of a bond, alkyl, —C(O)—, —C(O)O—, —OC(O)—, —SO₂—, —S(O)—, —C(S)—, —C(O)NR²⁷—, —NR²⁷C(O)—, —O—, —S—, —NR²⁷—, oxyalkylene, —C(R⁴⁰R⁴⁰)—, —P(O)(OR²⁶)O—, —P(O)(OR²⁶)—, bicycle, alkene, alkyne, haloalkyl, alkoxy, aryl, heterocycle, heteroaryl, lactic acid, glycolic acid, and carbocycle; each of which is optionally substituted with 1, 2, 3, or 4 substituents independently selected from R⁴⁰;
R²⁵ is alkyl, aryl, heteroaryl, or hydrogen;
R²⁶ is independently at each occurrence selected from the group consisting of hydrogen, alkyl, arylalkyl, heteroarylalkyl, alkene, alkyne, aryl, heteroaryl, and heterocycle;
R²⁷ is independently at each occurrence selected from the group consisting of hydrogen, alkyl, heterocycle, aryl, heteroaryl, —C(O)(alkyl, aryl, or heteroaryl), —C(O)O(alkyl, aryl, or heteroaryl), alkene, and alkyne;
R⁴⁰ is independently at each occurrence selected from the group consisting of hydrogen, R²⁷, alkyl, alkene, alkyne, fluoro, bromo, chloro, hydroxyl, alkoxy, azide, amino, cyano, —NH(alkyl), —N(alkyl)₂, —NHSO₂(alkyl), —N(alkyl)SO₂alkyl, —NHSO₂(aryl, heteroaryl or heterocycle), —N(alkyl)SO₂(aryl, heteroaryl or heterocycle), —NHSO₂alkenyl, —N(alkyl)SO₂alkenyl, —NHSO₂alkynyl, —N(alkyl)SO₂alkynyl, haloalkyl, aryl, heteroaryl, heterocycle, and cycloalkyl; and
R⁴¹ is alkyl, aryl, heteroaryl, or hydrogen.

2. The compound of claim 1, wherein the compound is of Formula: 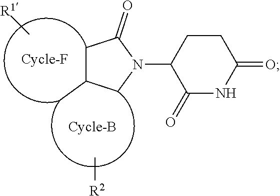
and wherein Cycle-F is phenyl substituted with 1, 2, or 3 substituents independently selected from R¹′.

3. The compound of claim 2, wherein Cycle-B is a fused ring selected from phenyl, 5- or 6-membered heteroaryl, 5- to 6-membered heterocycle, 5- to 6-membered cycloalkyl, and 5- to 6-membered cycloalkenyl, wherein Cycle-B is optionally substituted with 1, 2, or 3 substituents independently selected from R² as allowed by valence.

4. The compound of claim 1, wherein the compound is of Formula: 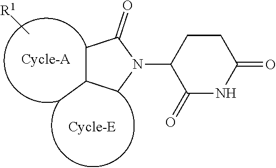
and wherein Cycle E is selected from the group consisting of:

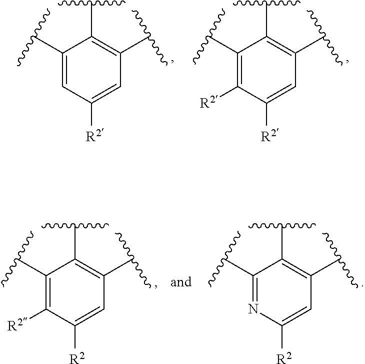

5. The compound of claim 4, wherein Cycle-A is a fused ring selected from phenyl, 5- or 6-membered heteroaryl, 5- to 6-membered heterocycle, 5- to 6-membered cycloalkyl, and 5- to 6-membered cycloalkenyl, wherein Cycle-A is optionally substituted with 1, 2, or 3 substituents independently selected from R¹ as allowed by valence.

6. The compound of claim 1, wherein R¹′, R²′ R¹, and R² are independently at each occurrence selected from the group consisting of alkyl, halogen, haloalkyl, —OR¹⁰, —SR¹⁰, —S(O)R¹², —SO₂R¹², —NR¹⁰R¹¹, heteroaryl, aryl, and heterocycle.

7. The compound of claim 1, wherein R¹′, R¹, and R² are independently at each occurrence

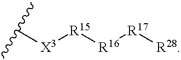

8. The compound of claim 1, wherein R¹′, R¹, and R² are independently at each occurrence

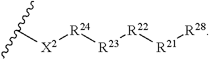

9. The compound of claim 1, wherein R¹′, R¹, and R² are independently at each occurrence

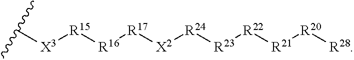

10. The compound of claim 1, wherein at least one of X³ and X² is a bond, —O—, —S—, or —NR²⁷—.

11. The compound of claim 1, wherein at least one of R¹, R¹⁶, R¹⁷, R²², R²³, and R²⁴ is a bond.

12. The compound of claim 1, wherein R³, R⁶, and R⁴ are independently at each occurrence selected from the group consisting of hydrogen, alkyl, and haloalkyl.

13. The compound of claim 1, wherein R³ and R⁶ are combined to form a one carbon attachment or a two-carbon attachment.

14. The compound of claim 1, wherein n is 1.

15. The compound of claim 1 selected from the group consisting of 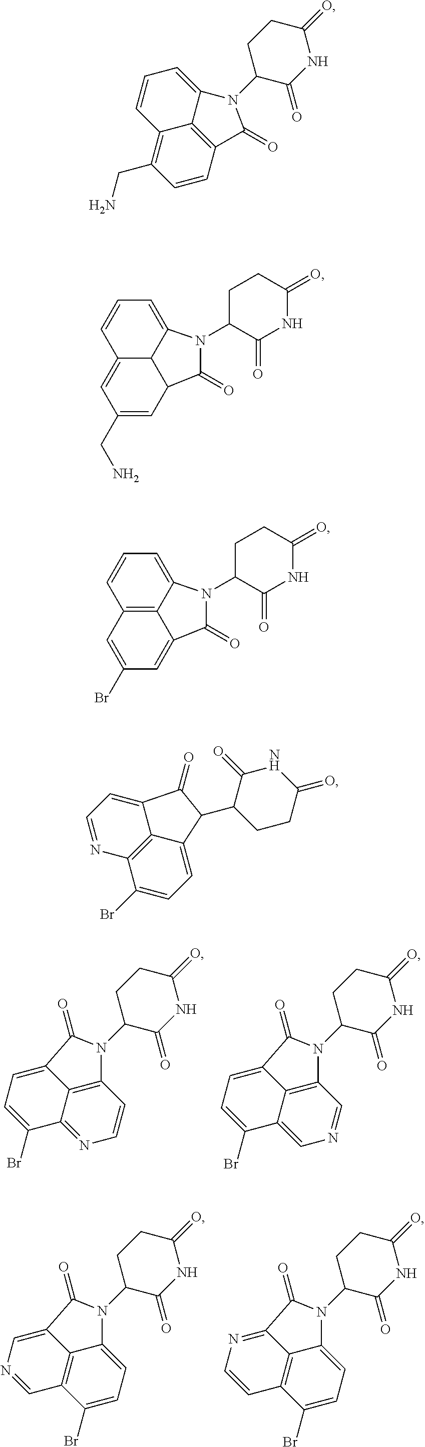 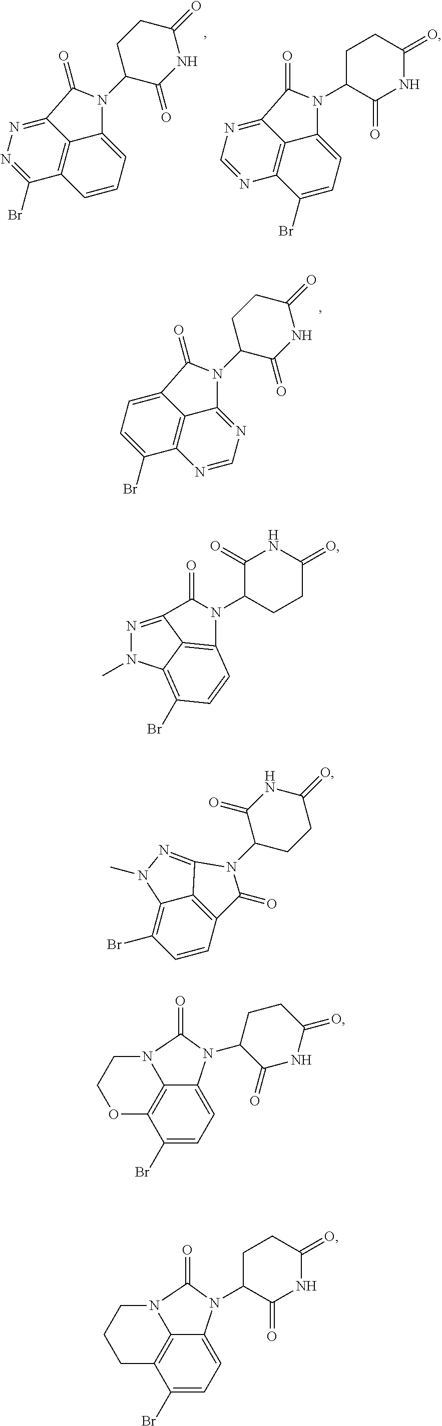 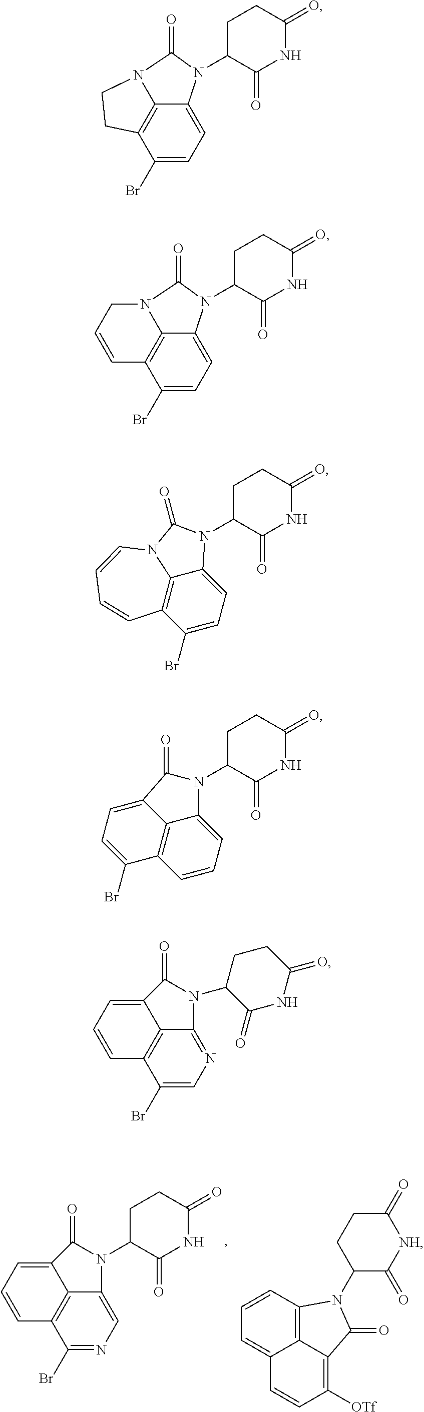 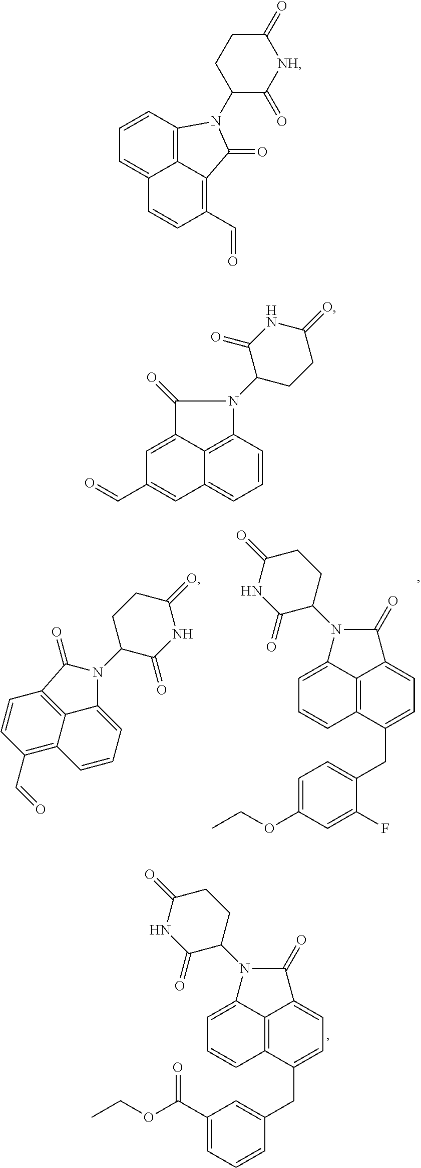 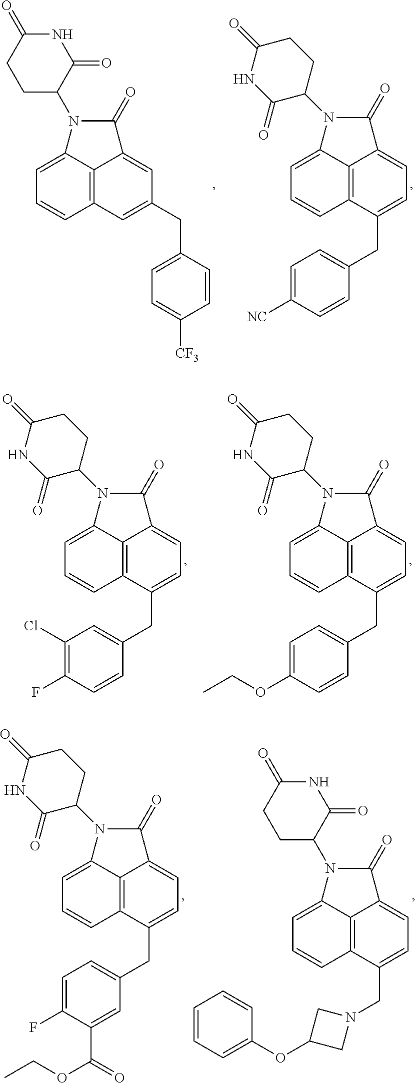 [SMILES: O=C1CCC(N2C(=O)c3cc(Cc4ccc(C(F)(F)F)cc4)cc4cccc2c34)C(=O)N1 ; N#Cc1ccc(Cc2ccc3c4c(cccc24)N(C2CCC(=O)NC2=O)C3=O)cc1 ; O=C1CCC(N2C(=O)c3ccc(Cc4ccc(F)c(Cl)c4)c4cccc2c34)C(=O)N1 ; CCOc1ccc(Cc2ccc3c4c(cccc24)N(C2CCC(=O)NC2=O)C3=O)cc1 ; CCOC(=O)c1cc(Cc2ccc3c4c(cccc24)N(C2CCC(=O)NC2=O)C3=O)ccc1F ; O=C1CCC(N2C(=O)c3ccc(CN4CC(Oc5ccccc5)C4)c4cccc2c34)C(=O)N1] 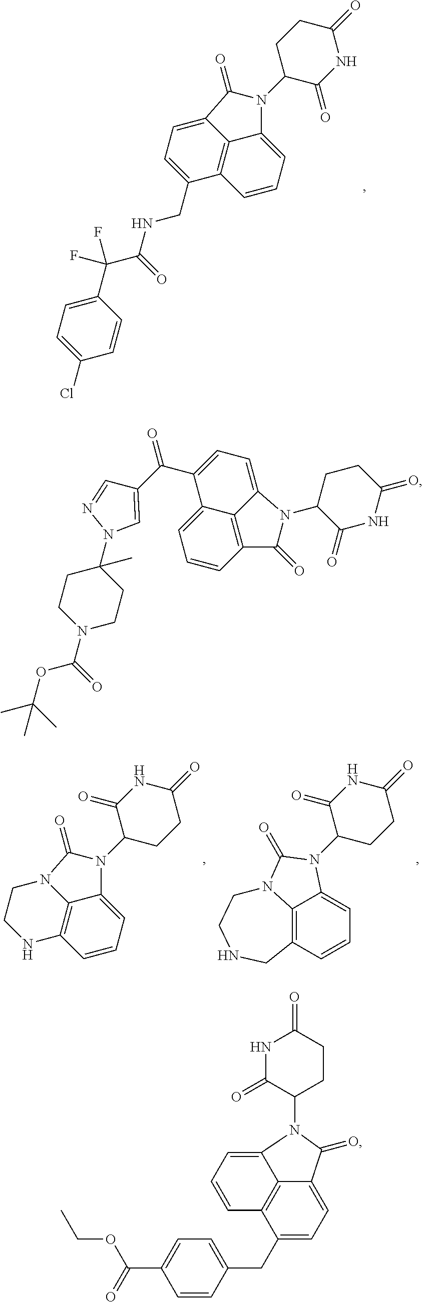 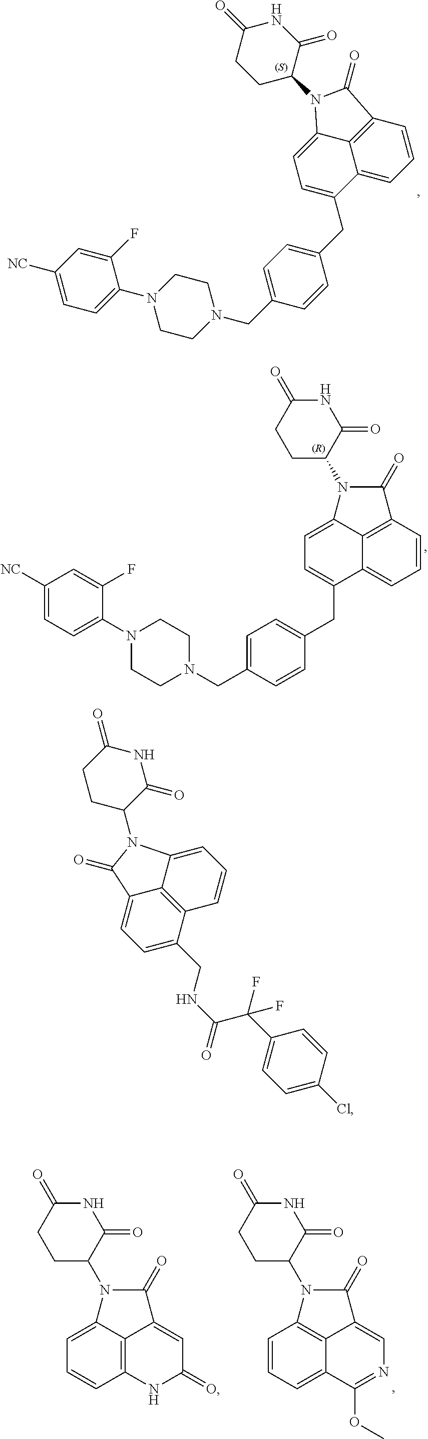 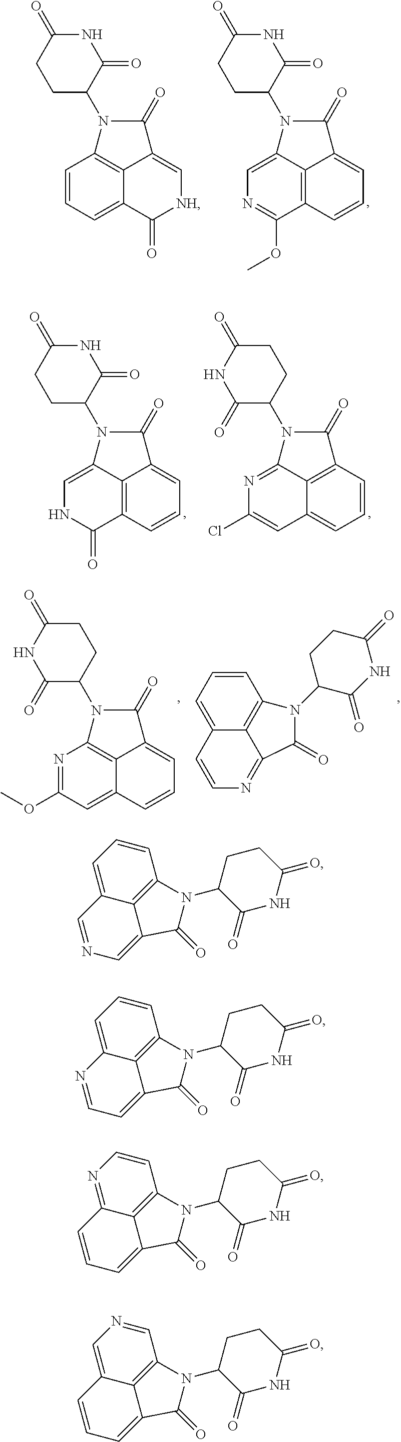 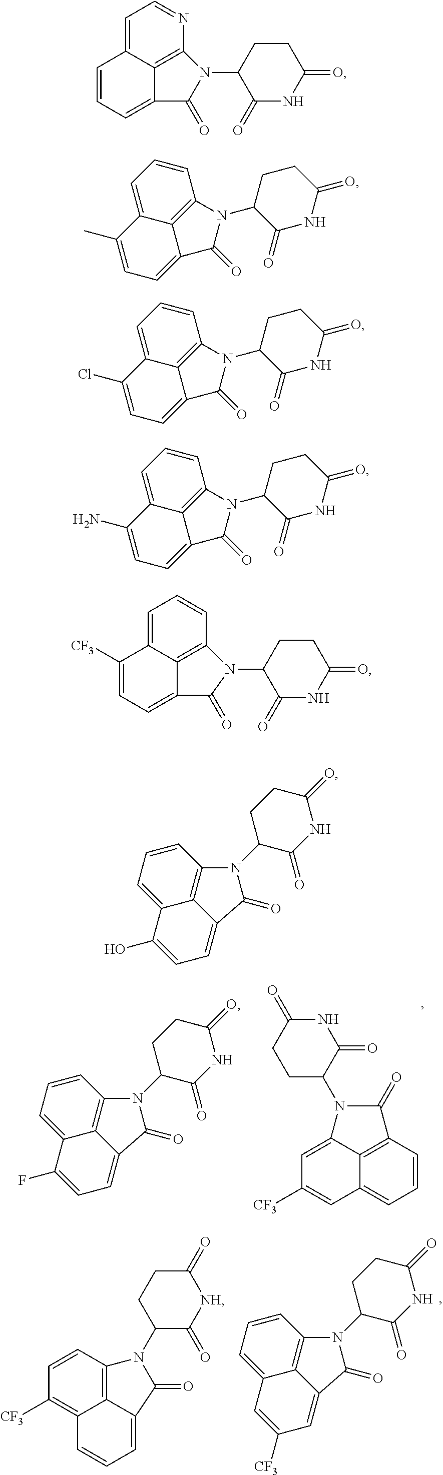 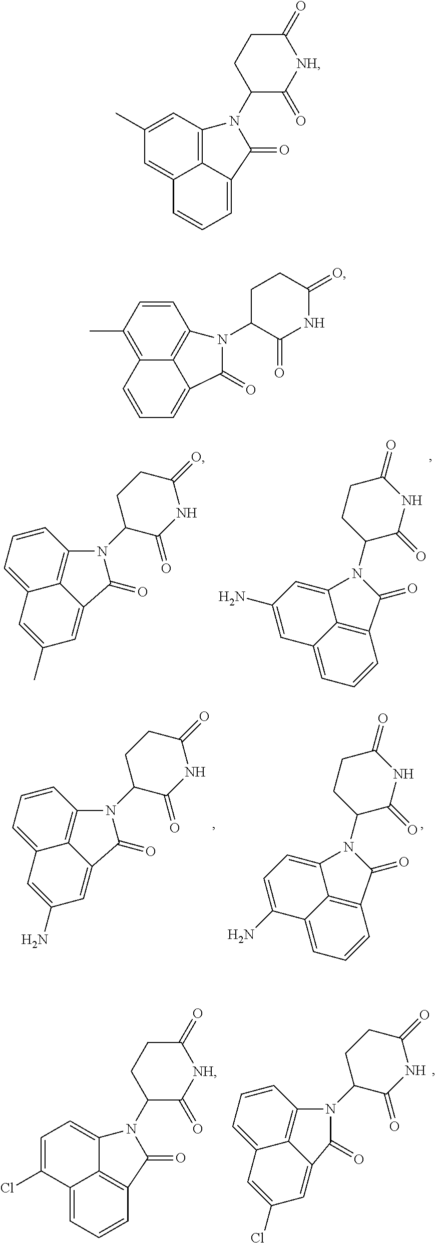 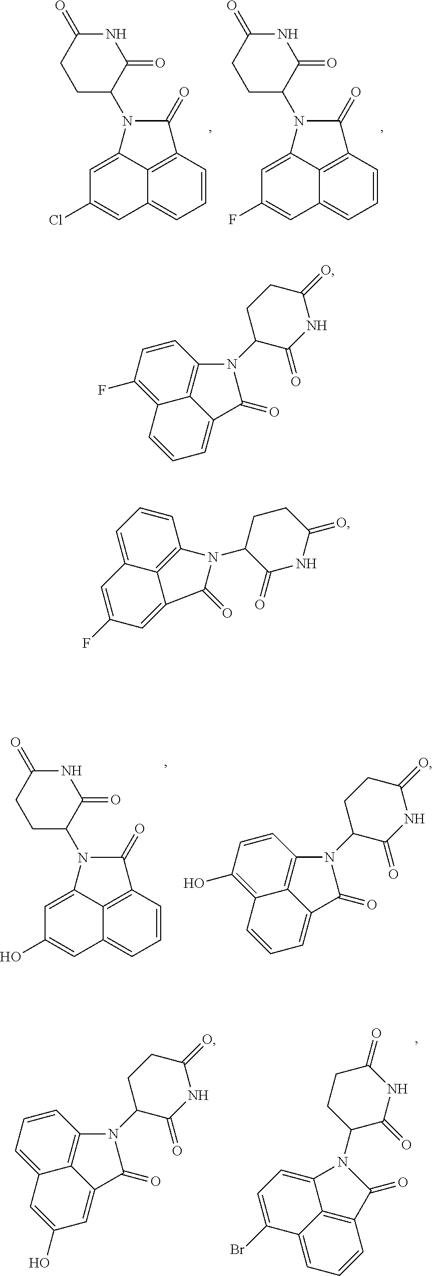 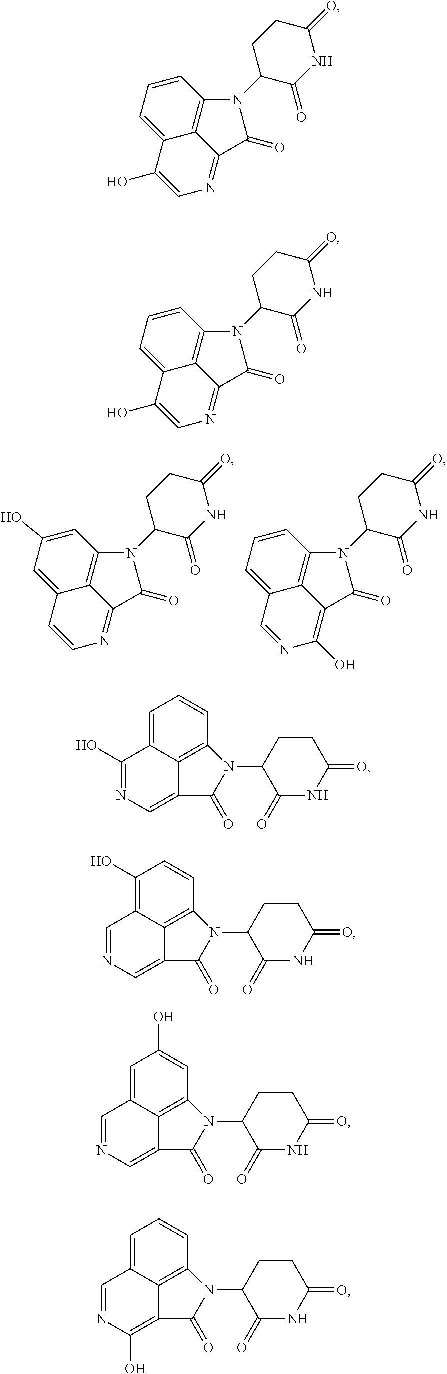 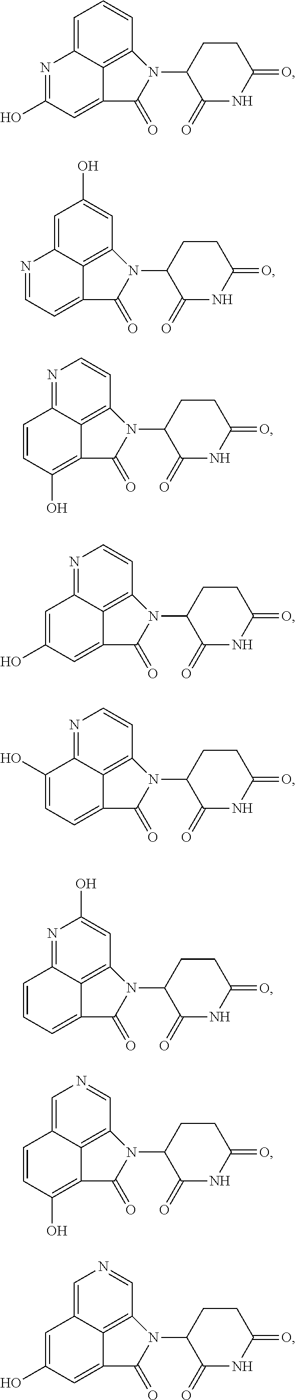 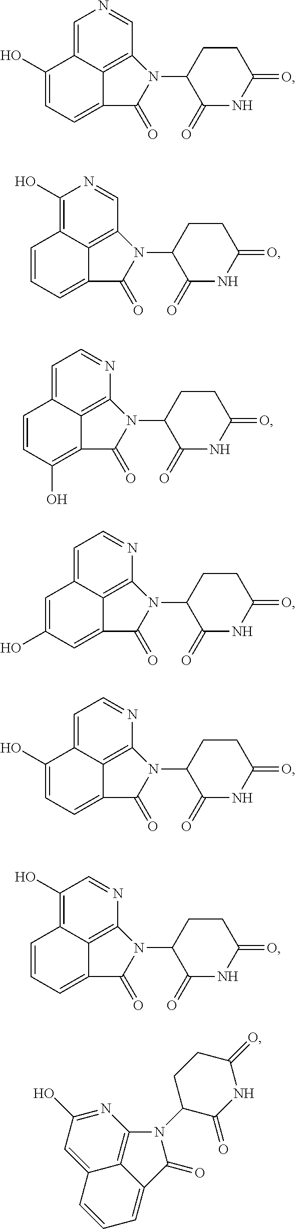 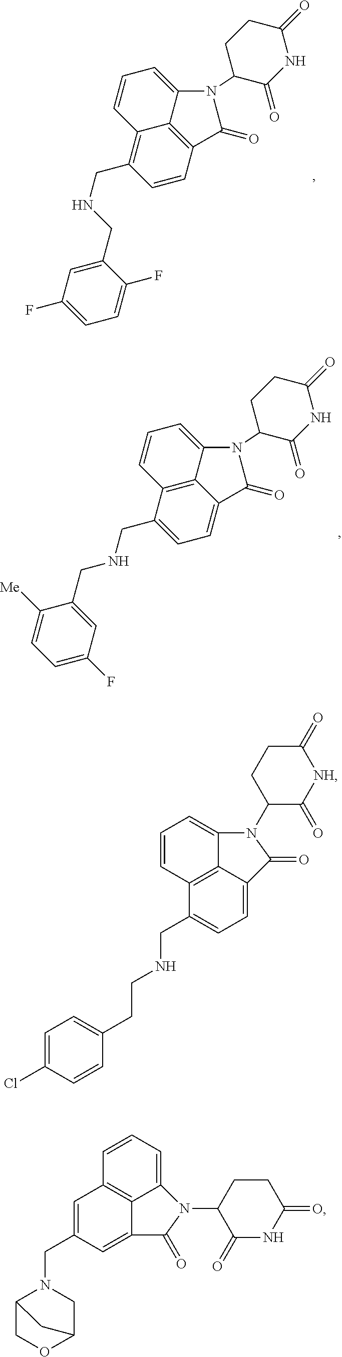 [SMILES: O=C1CCC(N2C(=O)c3ccc(CNCc4cc(F)ccc4F)c4cccc2c34)C(=O)N1 ; Cc1ccc(F)cc1CNCc1ccc2c3c(cccc13)N(C1CCC(=O)NC1=O)C2=O ; O=C1CCC(N2C(=O)c3ccc(CNCCc4ccc(Cl)cc4)c4cccc2c34)C(=O)N1 ; O=C1CCC(N2C(=O)c3cc(CN4CC5CC4CO5)cc4cccc2c34)C(=O)N1] 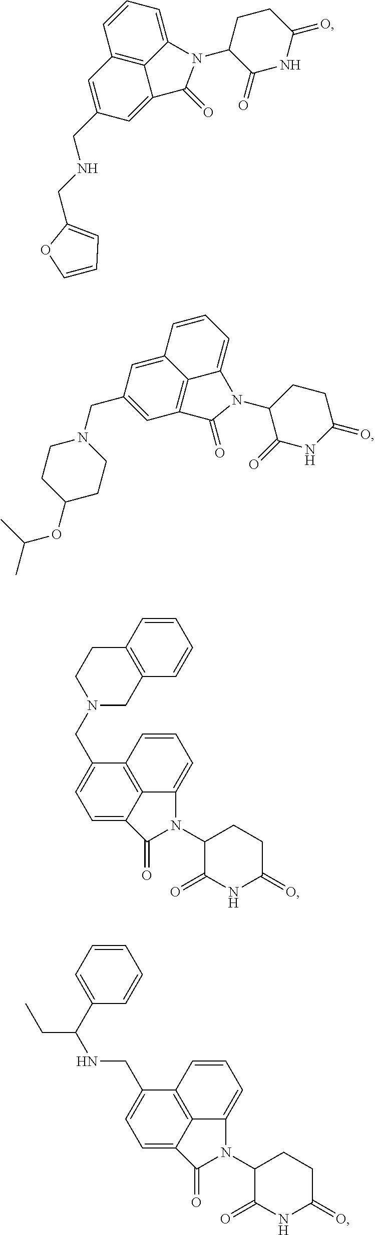 [SMILES: O=C1CCC(N2C(=O)c3cc(CNCc4ccco4)cc4cccc2c34)C(=O)N1 ; CC(C)OC1CCN(Cc2cc3c4c(cccc4c2)N(C2CCC(=O)NC2=O)C3=O)CC1 ; O=C1CCC(N2C(=O)c3ccc(CN4CCc5ccccc5C4)c4cccc2c34)C(=O)N1 ; CCC(NCc1ccc2c3c(cccc13)N(C1CCC(=O)NC1=O)C2=O)c1ccccc1] 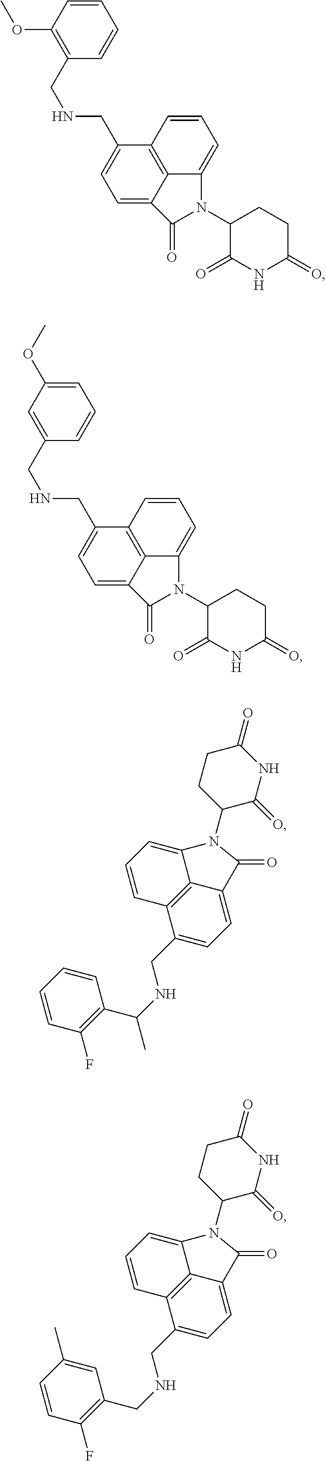 [SMILES: COc1ccccc1CNCc1ccc2c3c(cccc13)N(C1CCC(=O)NC1=O)C2=O ; COc1cccc(CNCc2ccc3c4c(cccc24)N(C2CCC(=O)NC2=O)C3=O)c1 ; CC(NCc1ccc2c3c(cccc13)N(C1CCC(=O)NC1=O)C2=O)c1ccccc1F ; Cc1ccc(F)c(CNCc2ccc3c4c(cccc24)N(C2CCC(=O)NC2=O)C3=O)c1] 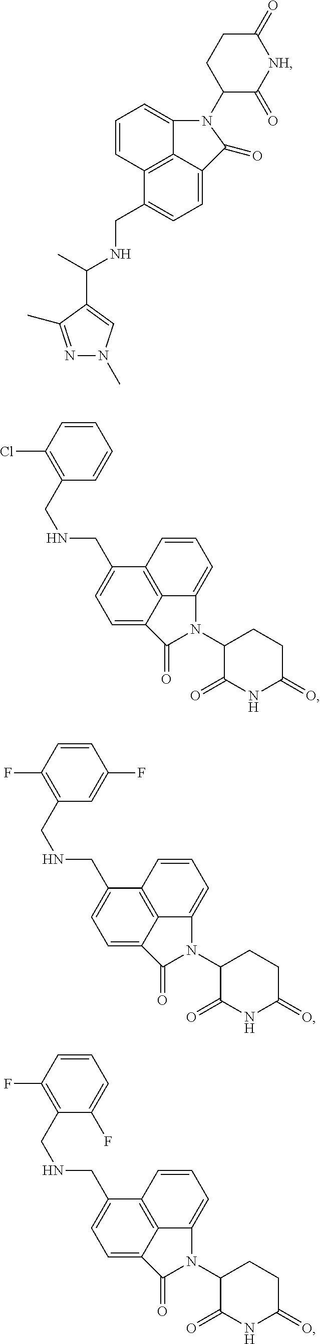 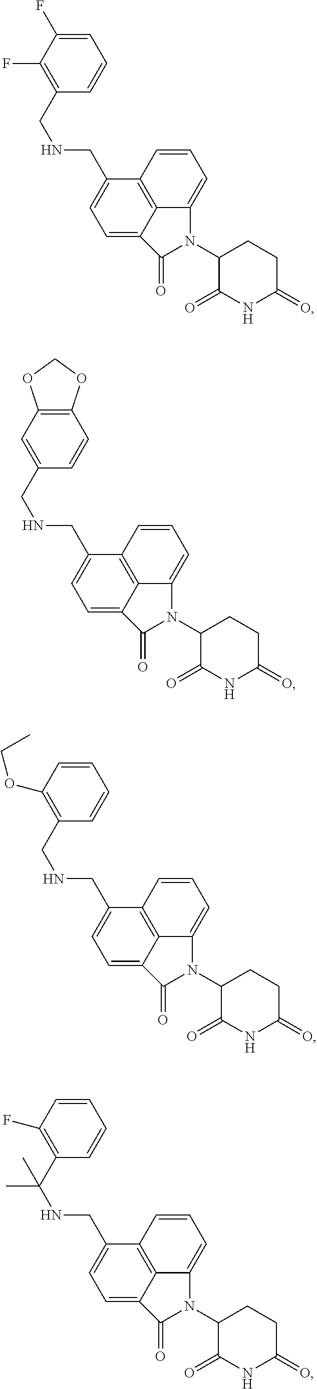 [SMILES: O=C1CCC(N2C(=O)c3ccc(CNCc4cccc(F)c4F)c4cccc2c34)C(=O)N1 ; O=C1CCC(N2C(=O)c3ccc(CNCc4ccc5c(c4)OCO5)c4cccc2c34)C(=O)N1 ; CCOc1ccccc1CNCc1ccc2c3c(cccc13)N(C1CCC(=O)NC1=O)C2=O ; CC(C)(NCc1ccc2c3c(cccc13)N(C1CCC(=O)NC1=O)C2=O)c1ccccc1F] 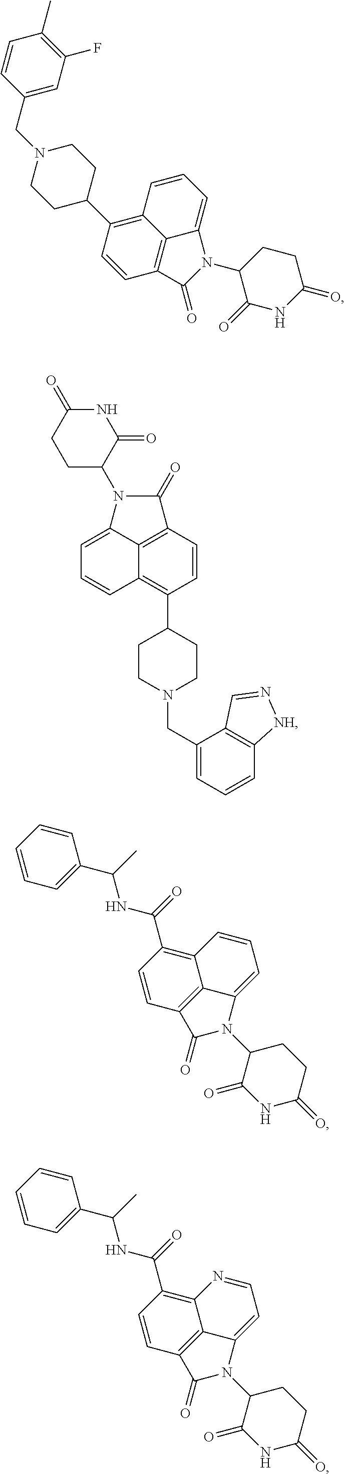 [SMILES: Cc1ccc(CN2CCC(c3ccc4c5c(cccc35)N(C3CCC(=O)NC3=O)C4=O)CC2)cc1F ; O=C1CCC(N2C(=O)c3ccc(C4CCN(Cc5cccc6[nH]ncc56)CC4)c4cccc2c34)C(=O)N1 ; CC(NC(=O)c1ccc2c3c(cccc13)N(C1CCC(=O)NC1=O)C2=O)c1ccccc1 ; CC(NC(=O)c1ccc2c3c(ccnc13)N(C1CCC(=O)NC1=O)C2=O)c1ccccc1] 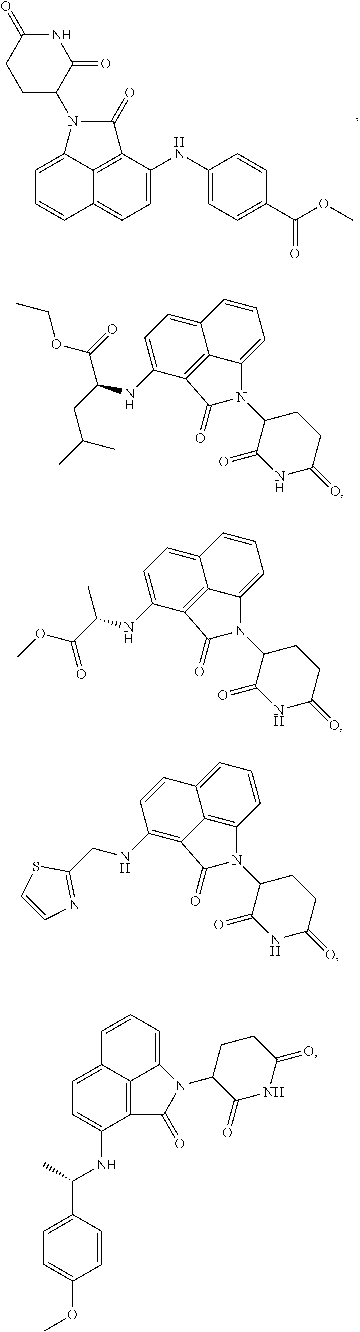 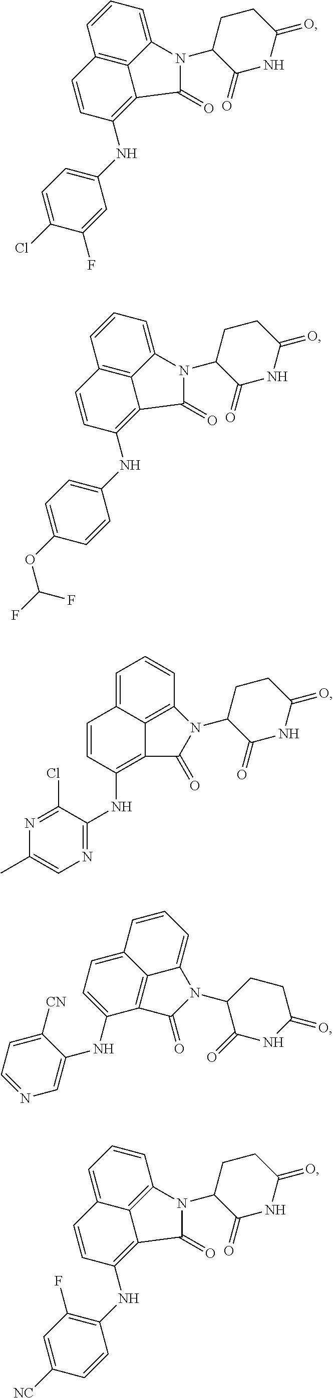 [SMILES: O=C1CCC(N2C(=O)c3c(Nc4ccc(Cl)c(F)c4)ccc4cccc2c34)C(=O)N1 ; O=C1CCC(N2C(=O)c3c(Nc4ccc(OC(F)F)cc4)ccc4cccc2c34)C(=O)N1 ; Cc1cnc(Nc2ccc3cccc4c3c2C(=O)N4C2CCC(=O)NC2=O)c(Cl)n1 ; N#Cc1ccncc1Nc1ccc2cccc3c2c1C(=O)N3C1CCC(=O)NC1=O ; N#Cc1ccc(Nc2ccc3cccc4c3c2C(=O)N4C2CCC(=O)NC2=O)c(F)c1] 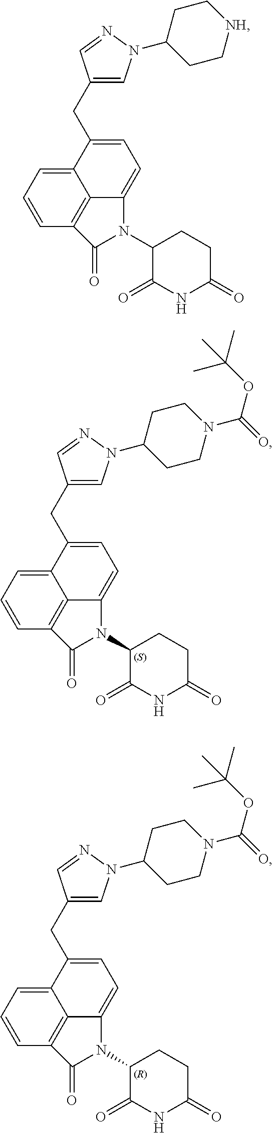 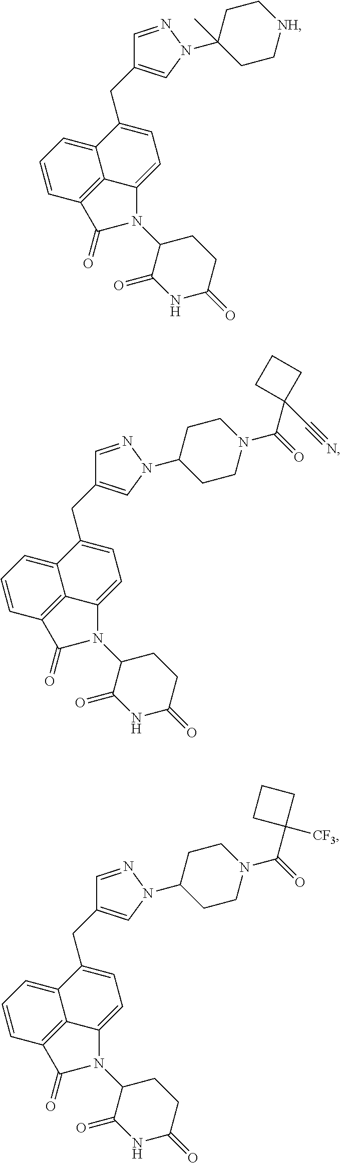 [SMILES: CC1(n2cc(Cc3ccc4c5c(cccc35)C(=O)N4C3CCC(=O)NC3=O)cn2)CCNCC1 ; N#CC1(C(=O)N2CCC(n3cc(Cc4ccc5c6c(cccc46)C(=O)N5C4CCC(=O)NC4=O)cn3)CC2)CCC1 ; O=C1CCC(N2C(=O)c3cccc4c(Cc5cnn(C6CCN(C(=O)C7(C(F)(F)F)CCC7)CC6)c5)ccc2c34)C(=O)N1] 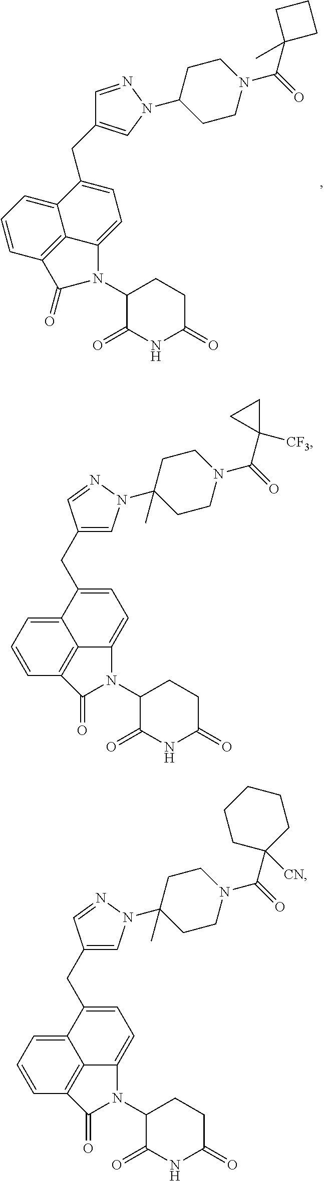 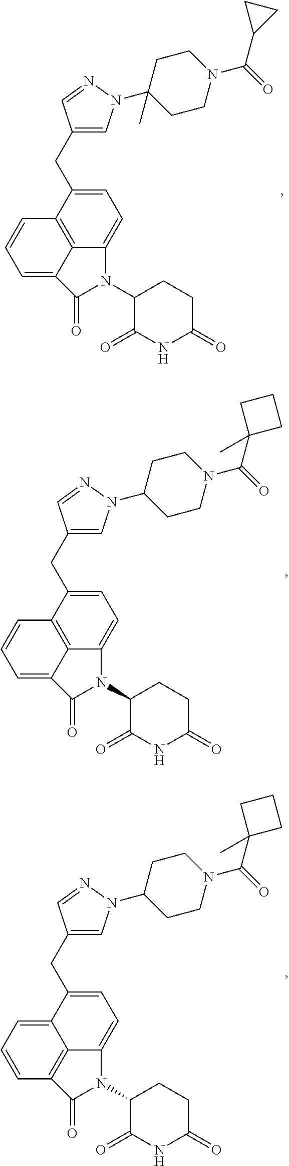 [SMILES: CC1(n2cc(Cc3ccc4c5c(cccc35)C(=O)N4C3CCC(=O)NC3=O)cn2)CCN(C(=O)C2CC2)CC1 ; CC1(C(=O)N2CCC(n3cc(Cc4ccc5c6c(cccc46)C(=O)N5[C@H]4CCC(=O)NC4=O)cn3)CC2)CCC1 ; CC1(C(=O)N2CCC(n3cc(Cc4ccc5c6c(cccc46)C(=O)N5[C@@H]4CCC(=O)NC4=O)cn3)CC2)CCC1] 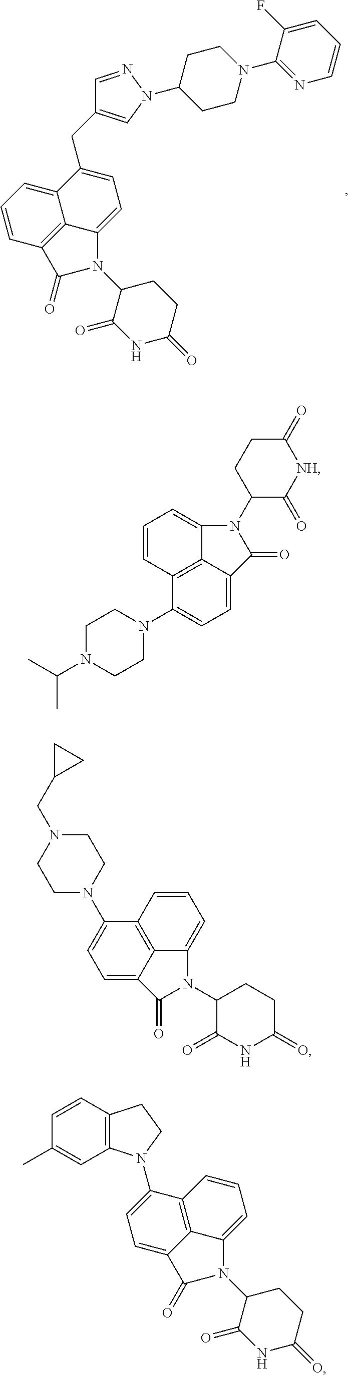 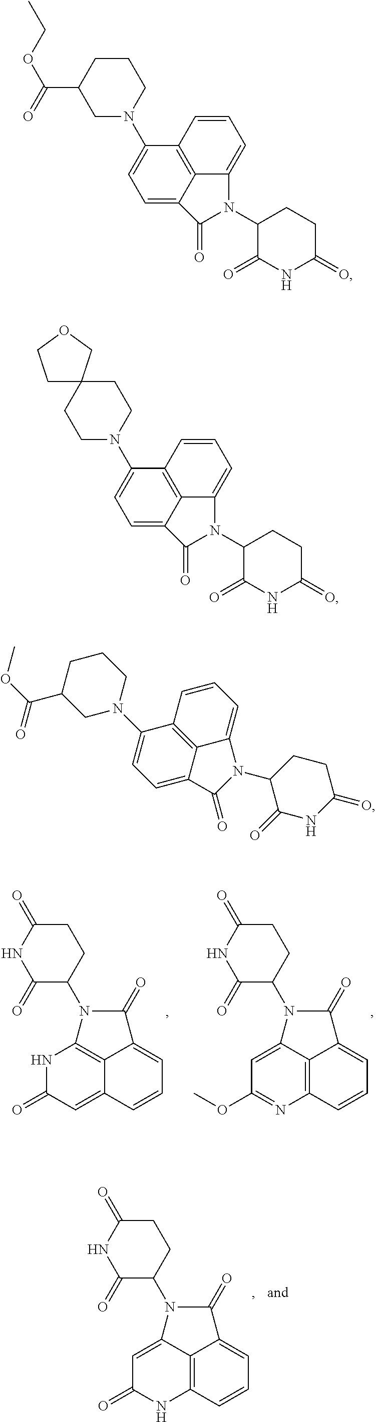 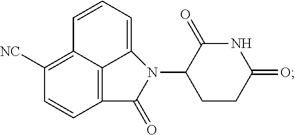 [SMILES: N#Cc1ccc2c3c(cccc13)N(C1CCC(=O)NC1=O)C2=O]
or pharmaceutically acceptable salt thereof.

16. A pharmaceutical composition comprising a compound of claim 1 and a pharmaceutically acceptable excipient.

17. A method of treating a medical disorder mediated by cereblon in a human comprising administering an effective amount of a compound of claim 1 or a pharmaceutically acceptable salt thereof to the human.

18. The method of claim 17, wherein the disorder is abnormal cellular proliferation.

19. The method of claim 17, wherein the disorder is a neurodegenerative disease.

20. The method of claim 17, wherein the disorder is an autoimmune disease.
